(19) United States
(12) Patent Application Publication
(10) Pub. No.: US 2026/0098053 A1
(43) Pub. Date: Apr. 9, 2026

(54) COMPOSITIONS AND METHODS FOR TREATING CNS DISORDERS

(71) Applicant: SAGE THERAPEUTICS, INC., Cambridge, MA (US)

(72) Inventors: Albert Jean Robichaud, Boston, MA (US); Francesco G. Salituro, Marlborough, MA (US); Maria Jesus Blanco-Pillado, Arlington, MA (US); Daniel La, Chestnut Hill, MA (US); Boyd L. Harrison, Princeton Junction, NJ (US); Marshall Lee Morningstar, Framingham, MA (US)

(21) Appl. No.: 19/234,399

(22) Filed: Jun. 11, 2025

Related U.S. Application Data
(63) Continuation of application No. 17/620,303.
(60) Provisional application No. 62/867,662, filed on Jun. 27, 2019.
(60) Provisional application No. 62/867,657, filed on Jun. 27, 2019.
(60) Provisional application No. 62/867,646, filed on Jun. 27, 2019.

(51) Int. Cl. C07J 67/00; C07J 7/00; C07J 43/00; C07J 63/00

(52) U.S. Cl. CPC C07J 67/00; C07J 7/009; C07J 43/003; C07J 63/00; C07J 63/002; C07J 63/008

(57) ABSTRACT

Provided herein is a compound of Formula (1-I): or a pharmaceutically acceptable salt thereof, wherein R^{2a}, R^{2b}, R³, R^{4a}, R^{4b}, R⁵, R^{6a}, R^{6b}, R^{11a}, R^{11b}, R^{16a}, R^{16b}, R¹⁹, R¹⁸, X, q, r, s, t, u, and n are defined herein. Also provided herein are pharmaceutical compositions comprising a compound of Formula (1-I) and methods of using the compounds, e.g. in the treatment of CNS-related disorders.

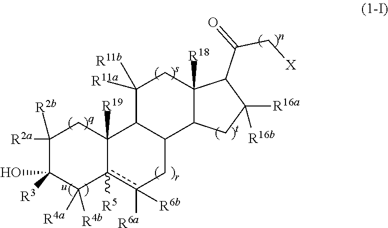

### CROSS REFERENCE TO RELATED APPLICATIONS

This application claims the benefit of, and priority to, U.S. provisional application Nos. 62/867,646, filed Jun. 27, 2019, 62/867,657, filed Jun. 27, 2019, 62/867,662, filed Jun. 27, 2019, the contents of each of which is hereby incorporated by reference herein in its entirety.

### BACKGROUND OF THE INVENTION

Brain excitability is defined as the level of arousal of an animal, a continuum that ranges from coma to convulsions, and is regulated by various neurotransmitters. In general, neurotransmitters are responsible for regulating the conductance of ions across neuronal membranes. At rest, the neuronal membrane possesses a potential (or membrane voltage) of approximately −70 mV, the cell interior being negative with respect to the cell exterior. The potential (voltage) is the result of ion (K⁺, Na⁺, Cl⁻, organic anions) balance across the neuronal semipermeable membrane. Neurotransmitters are stored in presynaptic vesicles and are released under the influence of neuronal action potentials. When released into the synaptic cleft, an excitatory chemical transmitter such as acetylcholine will cause membrane depolarization (change of potential occurs from −70 mV to −50 mV). This effect is mediated by postsynaptic nicotinic receptors which are stimulated by acetylcholine to increase membrane permeability to Na⁺ ions. The reduced membrane potential stimulates neuronal excitability in the form of a postsynaptic action potential.

In the case of the GABA receptor complex (GRC), the effect on brain excitability is mediated by 7-aminobutyric acid (GABA), a neurotransmitter. GABA has a profound influence on overall brain excitability because up to 40% of the neurons in the brain utilize GABA as a neurotransmitter. GABA regulates the excitability of individual neurons by regulating the conductance of chloride ions across the neuronal membrane. GABA interacts with its recognition site on the GRC to facilitate the flow of chloride ions down an electrochemical gradient of the GRC into the cell. An intracellular increase in the levels of this anion causes hyperpolarization of the transmembrane potential, rendering the neuron less susceptible to excitatory inputs, i.e., reduced neuron excitability. In other words, the higher the chloride ion concentration in the neuron, the lower the brain excitability and level of arousal.

It is well-documented that the GRC is responsible for the mediation of anxiety, seizure activity, and sedation. Thus, GABA and drugs that act like GABA or facilitate the effects of GABA (e.g., the therapeutically useful barbiturates and benzodiazepines (BZs), such as Valium®) produce their therapeutically useful effects by interacting with specific regulatory sites on the GRC. Accumulated evidence has now indicated that in addition to the benzodiazepine and barbiturate binding site, the GRC contains a distinct site for neuroactive steroids. See, e.g., Lan, N. C. et al., Neurochem. Res. (1991) 16:347-356.

Neuroactive steroids can occur endogenously. The most potent endogenous neuroactive steroids are 3α-hydroxy-5-reduced pregnan-20-one and 3α-21-dihydroxy-5-reduced pregnan-20-one, metabolites of hormonal steroids progesterone and deoxycorticosterone, respectively. The ability of these steroid metabolites to alter brain excitability was recognized in 1986 (Majewska, M. D. et al., Science 232:1004-1007 (1986); Harrison, N. L. et al., J Pharmacol. Exp. Ther. 241:346-353 (1987)).

New and improved compounds are needed that act as modulating agents for brain excitability, as well as agents for the prevention and treatment of CNS-related diseases. The compounds, compositions, and methods described herein are directed toward this end.

### SUMMARY OF THE INVENTION

Provided herein are compounds designed, for example, to act as GABA modulators. In some embodiments, such compounds are envisioned to be useful as therapeutic agents for treating a CNS-related disorder.

In an aspect, provided herein is a compound of Formula (1-I):

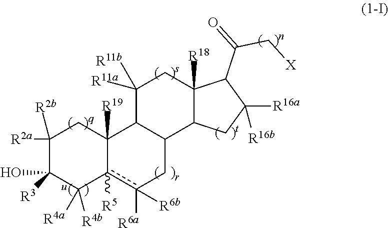

or a pharmaceutically acceptable salt thereof.

In some embodiments, the compound of Formula (1-I) is a compound of Formula (1-II-a):

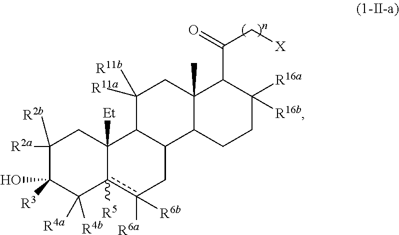

or a pharmaceutically acceptable salt thereof.

In some embodiments, the compound of Formula (1-I) is a compound of Formula (1-II-b):

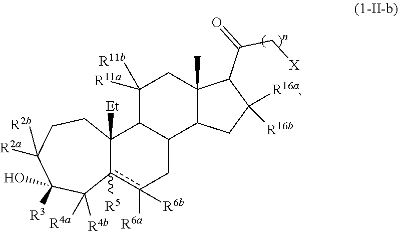

or a pharmaceutically acceptable salt thereof.

In some embodiments, the compound of Formula (1-I) is a compound of Formula (1-II-c):

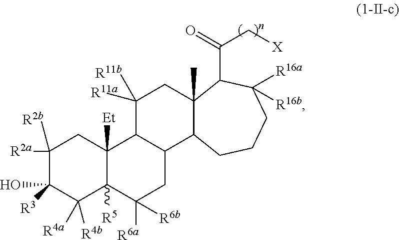

or a pharmaceutically acceptable salt thereof.

In some embodiments, the compound of Formula (1-I) is a compound of Formula (1(1-II-d)

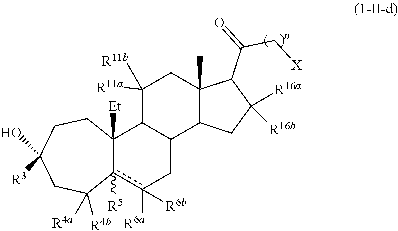

or a pharmaceutically acceptable salt thereof.

In some embodiments, the compound of Formula (1-I) is a compound of Formula (1-III-a):

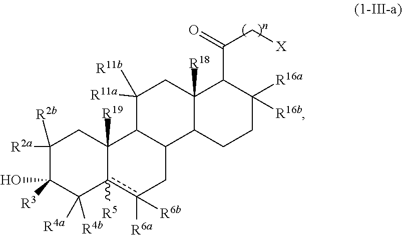

or a pharmaceutically acceptable salt thereof.

In some embodiments, the compound of Formula (1-I) is a compound of Formula (1-III-b):

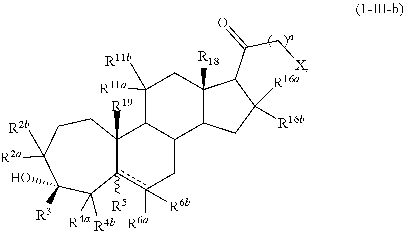

or a pharmaceutically acceptable salt thereof.

In some embodiments, the compound of Formula (1-I) is a compound of Formula (1-III-c):

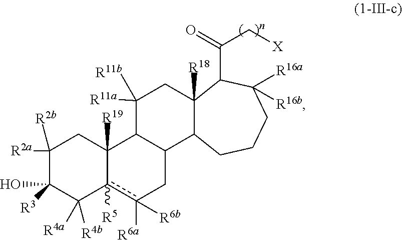

or a pharmaceutically acceptable salt thereof.

In some embodiments, the compound of Formula (1-I) is a compound of Formula (1-III-d):

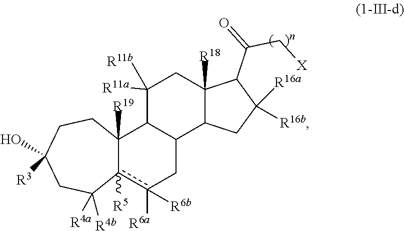

or a pharmaceutically acceptable salt thereof.

In some embodiments, the compound of Formula (1-I) is a compound of Formula (1-IV-a):

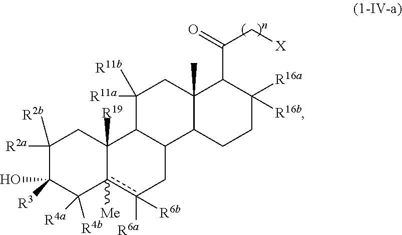

or a pharmaceutically acceptable salt thereof.

In some embodiments, the compound of Formula (1-I) is a compound of Formula (1-IV-b)

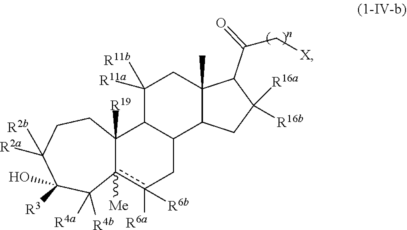

or a pharmaceutically acceptable salt thereof.

In some embodiments, the compound of Formula (1-I) is a compound of Formula (1-TV-c):

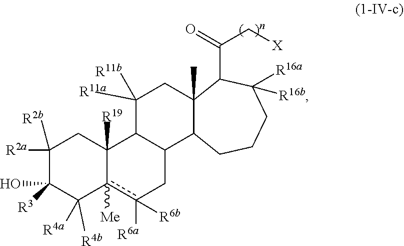

or a pharmaceutically acceptable salt thereof.

In some embodiments, the compound of Formula (1-I) is a compound of Formula (1-IV-d):

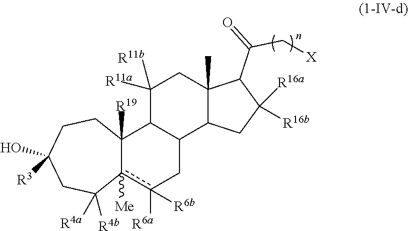

or a pharmaceutically acceptable salt thereof.

In some embodiments, the compound of Formula (1-I) is a compound of Formula (1-V-a):

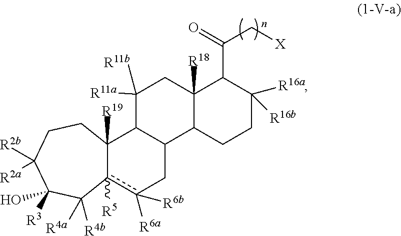

or a pharmaceutically acceptable salt thereof.

In some embodiments, the compound of Formula (1-I) is a compound of Formula (1-V-b):

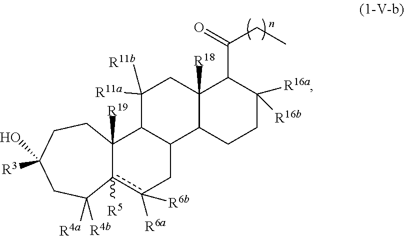

or a pharmaceutically acceptable salt thereof.

In some embodiments, the compound of Formula (1-I) is a compound of Formula (1-VT-b):

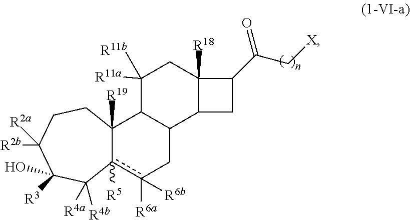

or a pharmaceutically acceptable salt thereof.

In some embodiments, the compound of Formula (1-I) is a compound of Formula (1-VI-b):

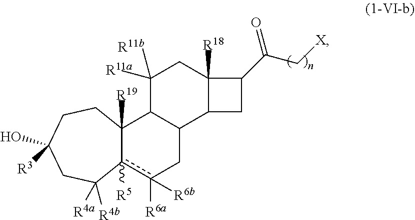

or a pharmaceutically acceptable salt thereof.

In some embodiments, the compound of Formula (1-I) is a compound of Formula (I-VII-a):

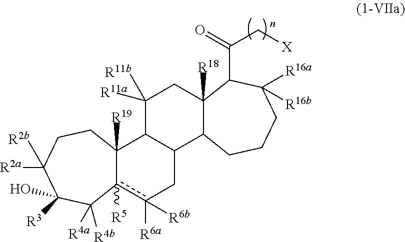

or a pharmaceutically acceptable salt thereof.

In some embodiments, the compound of Formula (1-I) is a compound of Formula (I-VII-b):

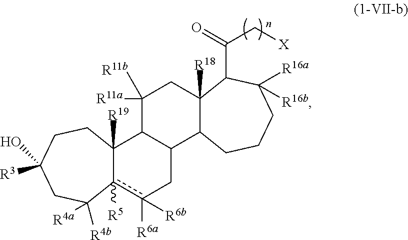

In some embodiments, the compound of Formula (1-I) is a compound of Formula (1-VIII-a):

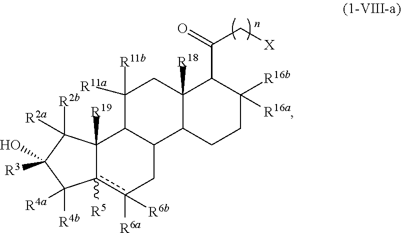

or a pharmaceutically acceptable salt thereof.

In some embodiments, the compound of Formula (1-I) is a compound of Formula (1-VIII-b):

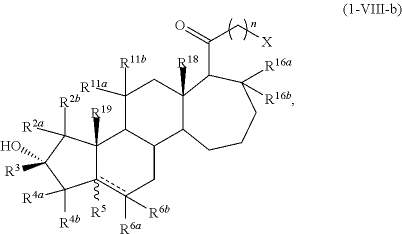

or a pharmaceutically acceptable salt thereof.

In some embodiments, the compound of Formula (1-I) is a compound of Formula (1-IX-a):

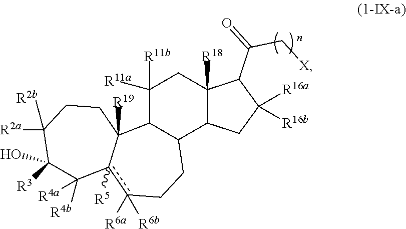

or a pharmaceutically acceptable salt thereof.

In some embodiments, the compound of Formula (1-I) is a compound of Formula (1-IX-b):

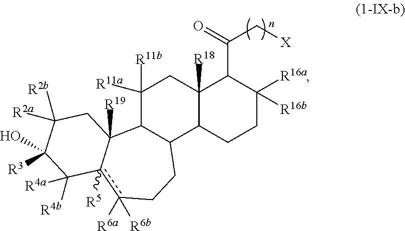

or a pharmaceutically acceptable salt thereof.

In some embodiments, the compound of Formula (1-I) is a compound of Formula (1-IX-c):

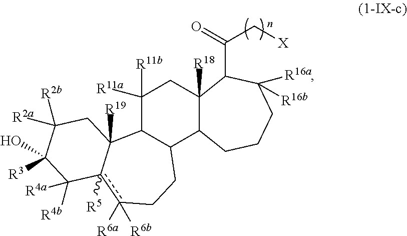

or a pharmaceutically acceptable salt thereof.

In some embodiments, the compound of Formula (1-I) is a compound of Formula (1-IX-d):

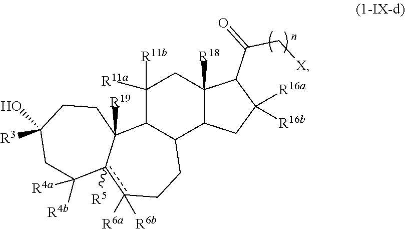

or a pharmaceutically acceptable salt thereof.

In some embodiments, the compound of Formula (1-I) is a compound of Formula (1-X-a):

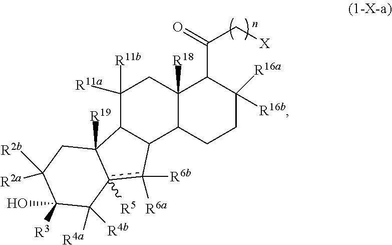

or a pharmaceutically acceptable salt thereof.

In some embodiments, the compound of Formula (1-I) is a compound of Formula (1-X-b):

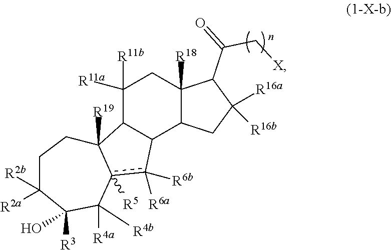

or a pharmaceutically acceptable salt thereof.

In some embodiments, the compound of Formula (1-I) is a compound of Formula (1-X-c):

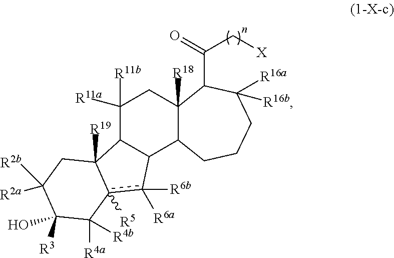

or a pharmaceutically acceptable salt thereof.

In some embodiments, the compound of Formula (1-I) is a compound of Formula (1-X-d):

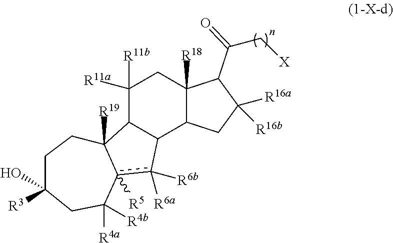

or a pharmaceutically acceptable salt thereof.

In some embodiments, the compound of Formula (1-I) is a compound of Formula (1-XI-a):

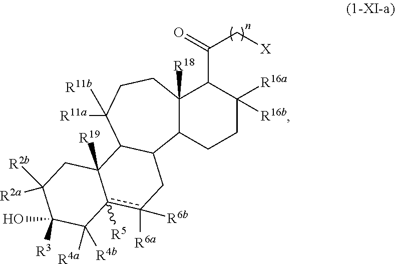

or a pharmaceutically acceptable salt thereof.

In some embodiments, the compound of Formula (1-I) is a compound of Formula (1-XI-b):

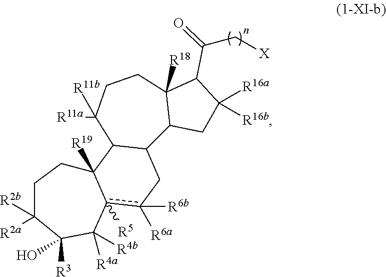

or a pharmaceutically acceptable salt thereof.

In some embodiments, the compound of Formula (1-I) is a compound of Formula (1-XI-c):

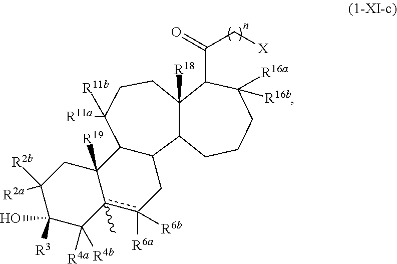

or a pharmaceutically acceptable salt thereof.

In some embodiments, the compound of Formula (1-I) is a compound of Formula (1-XI-d):

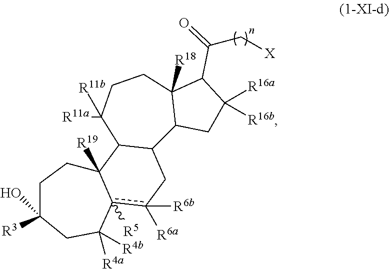

or a pharmaceutically acceptable salt thereof.

In some embodiments, the compound of Formula (1-I) is a compound of Formula (1-XII-a):

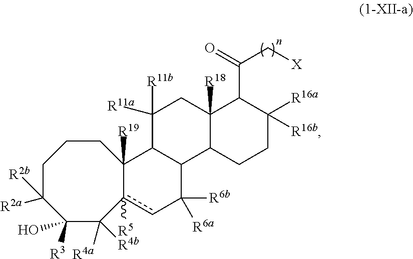

or a pharmaceutically acceptable salt thereof.

In some embodiments, the compound of Formula (1-I) is a compound of Formula (1-XII-b):

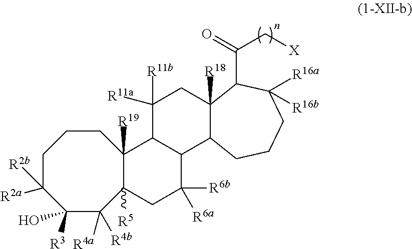

or a pharmaceutically acceptable salt thereof.

In some embodiments, the compound of Formula (1-I) is a compound of Formula (1-XIII-a):

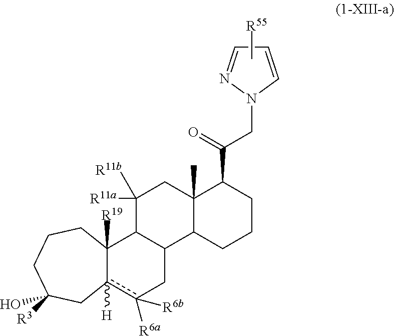

or a pharmaceutically acceptable salt thereof.

In some embodiments, the compound of Formula (1-I) is a compound of Formula (1-XIII-b)

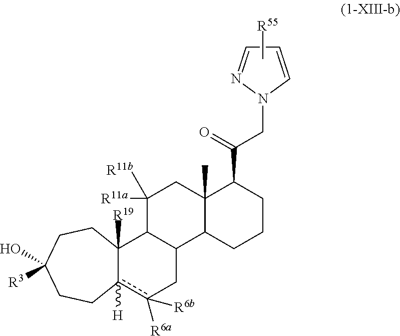

or a pharmaceutically acceptable salt thereof.

In some embodiments, the compound of Formula (1-I) is a compound of Formula (1-XIV-a):

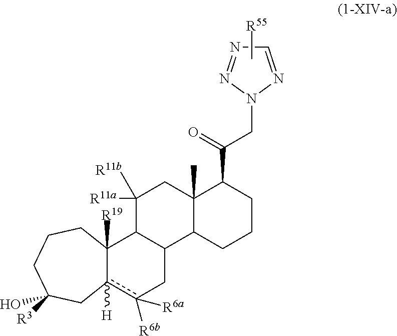

or a pharmaceutically acceptable salt thereof.

In some embodiments, the compound of Formula (1-I) is a compound of Formula (1-XIV-b):

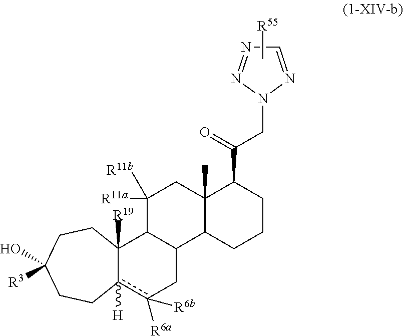

or a pharmaceutically acceptable salt thereof.

In an aspect, provided herein are compounds of Formula 2-I:

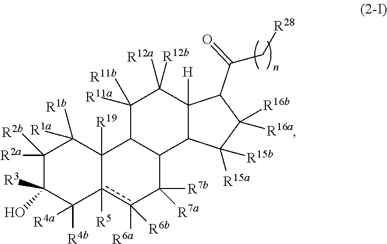

or a pharmaceutically acceptable salt thereof.

In some embodiments, the compound of Formula 2-I is a compound of Formula 2-Ia or Formula 2-Ib:

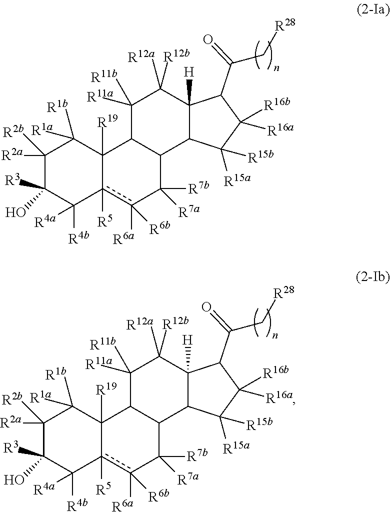

or a pharmaceutically acceptable salt thereof.

In some embodiments, the compound of Formula 2-I is a compound of Formula 2-Iaa or Formula 2-Iab:

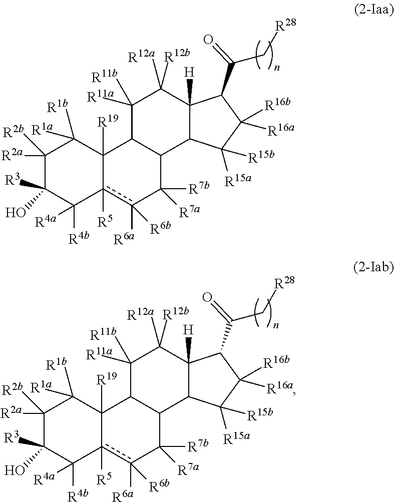

or a pharmaceutically acceptable salt thereof.

In some embodiments, provided herein are compounds of Formula 2-II:

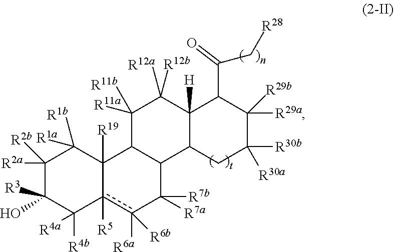

or a pharmaceutically acceptable salt thereof.

In some embodiments, the compound of Formula 2-II is a compound of Formula 2-IIa

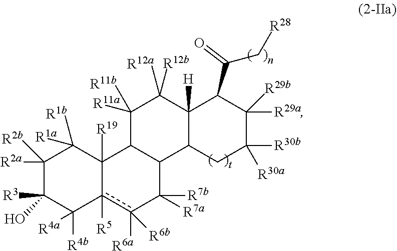

or a pharmaceutically acceptable salt thereof.

In some embodiments, provided herein are compounds of Formula 2-III:

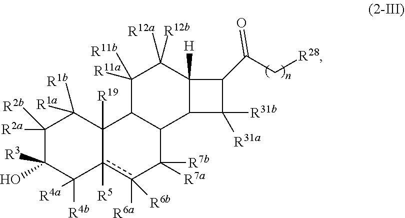

or a pharmaceutically acceptable salt thereof.

In some embodiments, the compound of Formula 2-III is a compound of Formula 2-IIIa

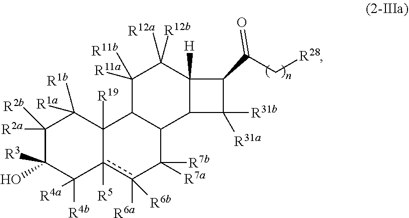

or a pharmaceutically acceptable salt thereof.

In certain embodiments, provided herein are compounds of Formula 2-IVa or Formula 2-IVb:

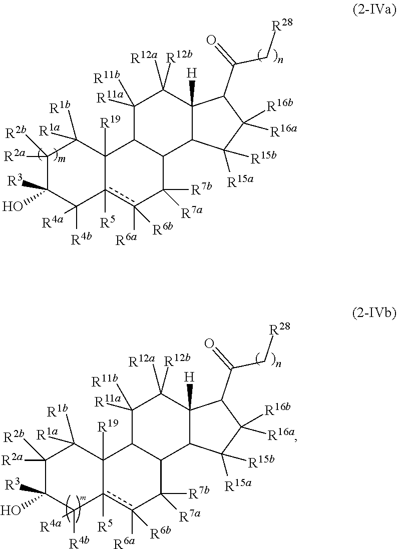

or a pharmaceutically acceptable salt thereof.

In some embodiments, the compound of Formula 2-IV is a compound of Formula 2-IVaa or Formula 2-IVba:

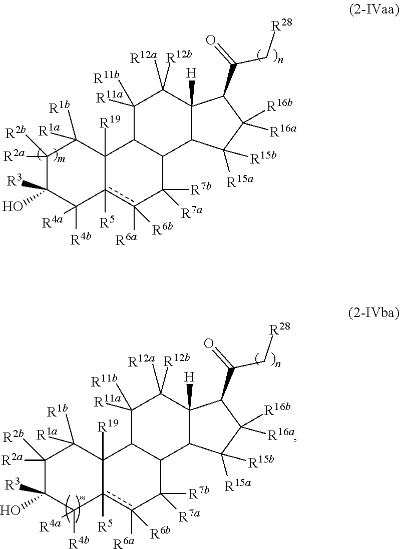

or a pharmaceutically acceptable salt thereof.

In embodiments, provided herein are compounds of Formula 2-V:

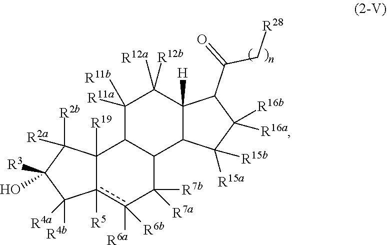

or a pharmaceutically acceptable salt thereof.

In some embodiments, the compound of Formula 2-V is a compound of Formula 2-Va:

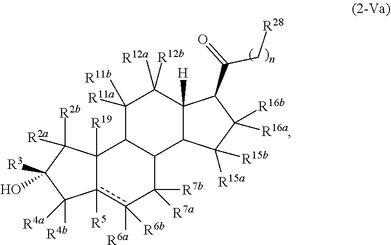

or a pharmaceutically acceptable salt thereof.

In embodiments, provided herein are compounds of Formula 2-VI:

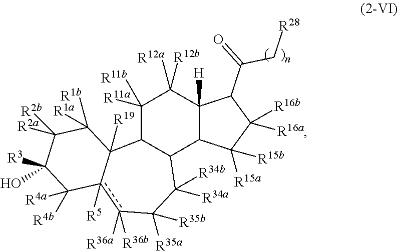

or a pharmaceutically acceptable salt thereof.

In some embodiments, the compound of Formula 2-VI is a compound of Formula 2-VIa:

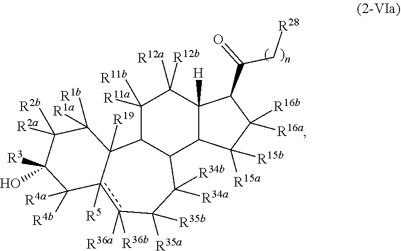

or a pharmaceutically acceptable salt thereof.

In embodiments, provided herein are compounds of Formula 2-VII:

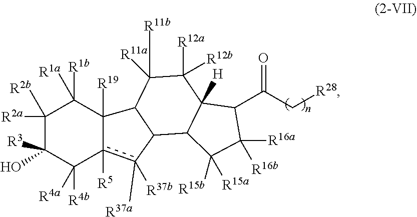

or a pharmaceutically acceptable salt thereof.

In some embodiments, the compound of Formula 2-VII is a compound of Formula 2-VIIa:

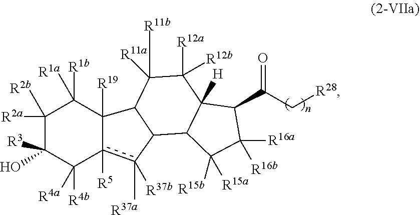

or a pharmaceutically acceptable salt thereof.

In an aspect, provided herein is a compound of Formula 3-T:

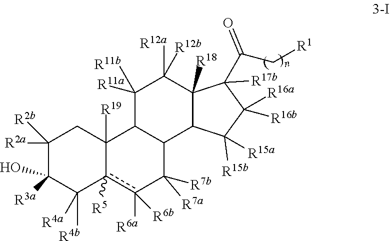

or a pharmaceutically acceptable salt thereof.

In some embodiments, the compound of Formula 3-I is a compound of Formula 3-IIa or Formula 3-IIb:

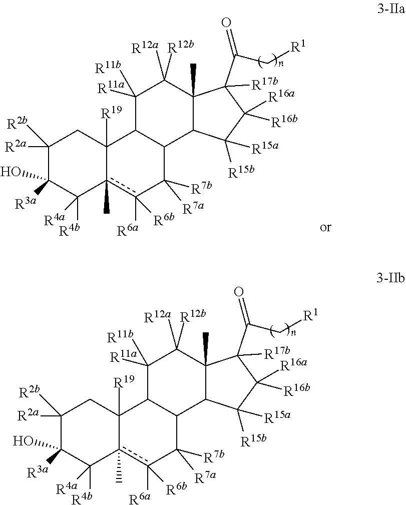

or a pharmaceutically acceptable salt thereof.

In some embodiments, the compound of Formula 3-III:

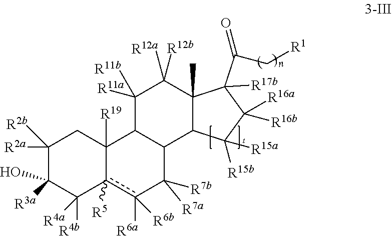

or a pharmaceutically acceptable salt thereof.

In some embodiments, the compound of Formula 3-IV:

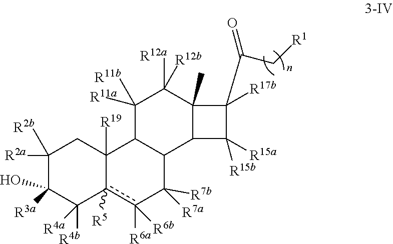

or a pharmaceutically acceptable salt thereof.

In some embodiments, the compound of Formula Va or Formula 3-Vb:

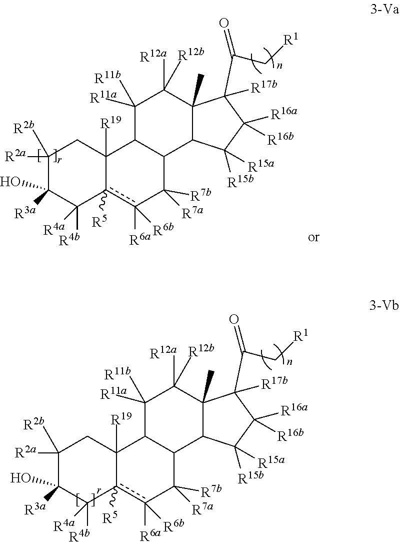

or a pharmaceutically acceptable salt thereof.

In some embodiments, the compound of Formula 3-VI:

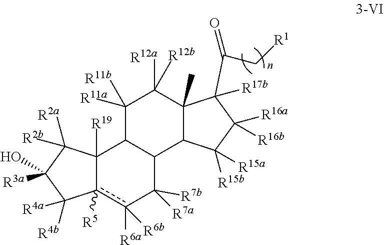

or a pharmaceutically acceptable salt thereof.

In some embodiments, the compound of Formula 3-VII:

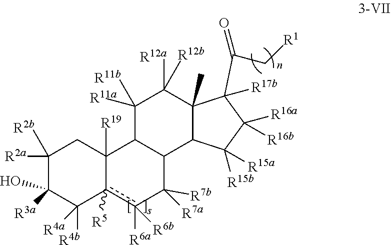

or a pharmaceutically acceptable salt thereof.

In some embodiments, the compound of Formula 3-VIII:

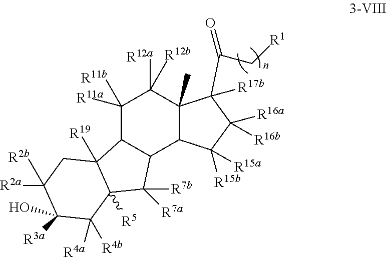

or a pharmaceutically acceptable salt thereof.

In some embodiments, the compound of Formula 3-IX:

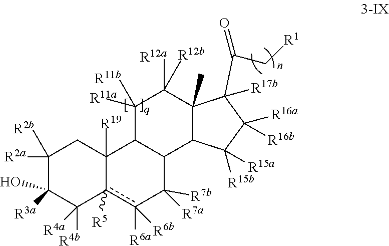

or a pharmaceutically acceptable salt thereof.

In some embodiments, the compound of Formula 3-1a:

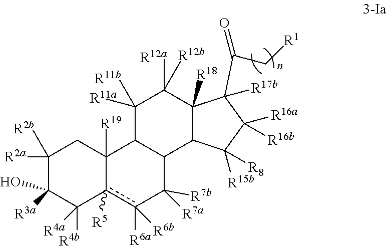

or a pharmaceutically acceptable salt thereof.

In some embodiments, the compound of Formula 3-IIaa or Formula 3-IIba:

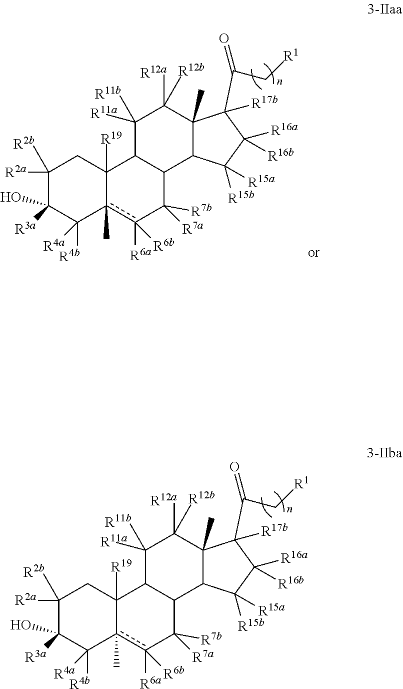

or a pharmaceutically acceptable salt thereof.

In some embodiments, the compound of Formula 3-IIIa:

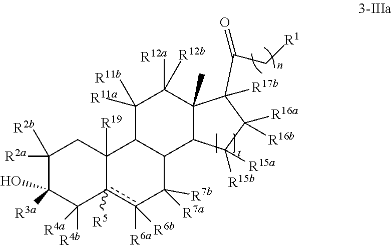

or a pharmaceutically acceptable salt thereof.

In some embodiments, the compound of Formula 3-IVa:

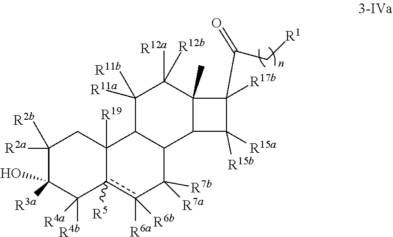

or a pharmaceutically acceptable salt thereof.

In some embodiments, the compound of Formula 3-Vac or Formula 3-Vacc:

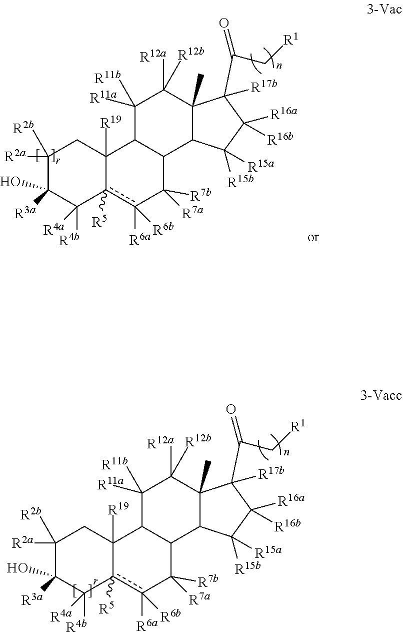

or a pharmaceutically acceptable salt thereof.

In some embodiments, the compound of Formula 3-VIac:

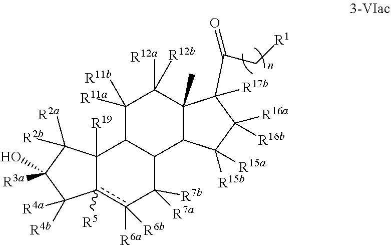

or a pharmaceutically acceptable salt thereof.

In some embodiments, the compound of Formula 3-VIIac:

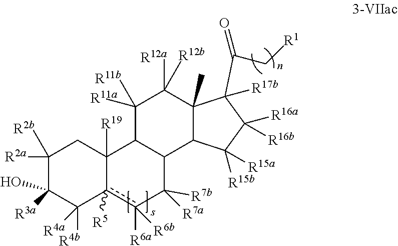

or a pharmaceutically acceptable salt thereof.

In some embodiments, the compound of Formula 3-VIIIac:

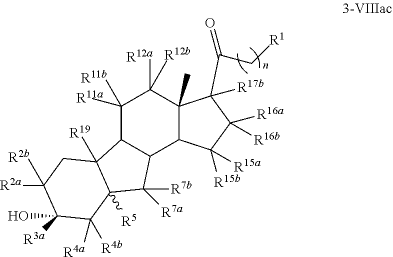

or a pharmaceutically acceptable salt thereof.

In some embodiments, the compound of Formula 3-IXac.

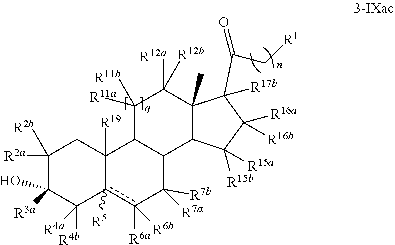

or a pharmaceutically acceptable salt thereof.

In one aspect, provided herein is a pharmaceutical composition comprising a compound described herein (e.g., a compound of Formula (1-I)) or a pharmaceutically acceptable salt thereof, and a pharmaceutically acceptable excipient. In certain embodiments, the compound of the present invention is provided in an effective amount in the pharmaceutical composition. In certain embodiments, the compound of the present invention is provided in a therapeutically effective amount.

In some embodiments, a method of treating a CNS-related disorder in a subject in need thereof, comprises administering to the subject an effective amount of a compound described herein or a pharmaceutically acceptable salt thereof. In some embodiments, the CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, or status epilepticus. In some embodiments, the CNS-related disorder is depression. In some embodiments, the CNS-related disorder is postpartum depression. In some embodiments, the CNS-related disorder is major depressive disorder. In some embodiments, the major depressive disorder is moderate major depressive disorder. In some embodiments, the major depressive disorder is severe major depressive disorder.

In some embodiments, the compound is selected from the group consisting of the compounds identified in Tables 1-3 herein.

Compounds of the present invention as described herein, act, in certain embodiments, as GABA modulators, e.g., effecting the GABAA receptor in either a positive or negative manner. As modulators of the excitability of the central nervous system (CNS), as mediated by their ability to modulate GABAA receptor, such compounds are expected to have CNS-activity.

Thus, in another aspect, provided are methods of treating a CNS-related disorder in a subject in need thereof, comprising administering to the subject an effective amount of a compound of the present invention. In certain embodiments, CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, or status epilepticus. In certain embodiments, the CNS-related disorder is depression. In certain embodiments, the CNS-related disorder is postpartum depression. In certain embodiments, the CNS-related disorder is major depressive disorder. In certain embodiments, the major depressive disorder is moderate major depressive disorder. In certain embodiments, the major depressive disorder is severe major depressive disorder. In certain embodiments, the compound is administered orally, subcutaneously, intravenously, or intramuscularly. In certain embodiments, the compound is administered orally. In certain embodiments, the compound is administered chronically. In certain embodiments, the compound is administered continuously, e.g., by continuous intravenous infusion.

### DETAILED DESCRIPTION OF CERTAIN EMBODIMENTS OF THE INVENTION

As generally described herein, the present invention provides compounds designed, for example, to act as GABAA receptor modulators. In certain embodiments, such compounds are envisioned to be useful as therapeutic agents for treating a CNS-related disorder (e.g., a disorder as described herein, for example depression, such as post-partum depression or major depressive disorder).

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Isomers, e.g., stereoisomers, can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

“Stereoisomers”: It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-position/center/carbon compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The term “diastereomierically pure” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of a single diastereomer. Methods for determining diastereomeric and enantiomeric purity are well-known in the art. Diastereomeric purity can be determined by any analytical method capable of quantitatively distinguishing between a compound and its diastereomers, such as high performance liquid chromatography (HPLC).

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₅, C₄₋₅, and C₅₋₆ alkyl.

The following terms are intended to have the meanings presented therewith below and are useful in understanding the description and intended scope of the present invention.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like. When a range or number of carbons is provided for a particular alkylene group, it is understood that the range or number refers to the range or number of carbons in the linear carbon divalent chain. Alkylene groups may be substituted or unsubstituted with one or more substituents as described herein.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₈ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₅), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₅ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and lor 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group substituted with one or more of groups selected from halo, C₁-C₈ alkyl, C₁-C₈ haloalkyl, cyano, hydroxy, C₁-C₈ alkoxy, and amino.

Examples of representative substituted aryls include the following

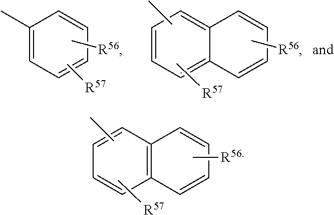

wherein one of R⁵⁶ and R⁵⁷ may be hydrogen and at least one of R⁵⁶ and R⁵⁷ is each independently selected from C₁-C₅ alkyl, C₁-C₅ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁-C₈ alkyl, C₁-C₄haloalkyl, C₃-C₁₀ cycloalkyl, 4-10 membered heterocyclyl, C₆-C₁₀ aryl, substituted C₆₋₁₀ aryl, 5-10 membered heteroaryl, or substituted 5-10 membered heteroaryl.

“Fused aryl” refers to an aryl having two of its ring carbon in common with a second aryl or heteroaryl ring or with a carbocyclyl or heterocyclyl ring.

“Heteroaryl” refers to a radical of a 5-10 membered monocyclic or bicyclic 4n+2 aromatic ring system (e.g., having 6 or 10 it electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

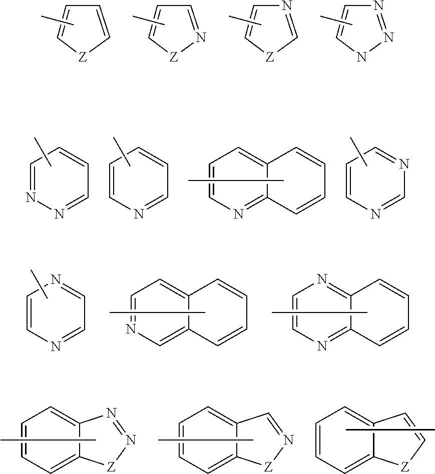

wherein each Z is selected from carbonyl, N, NR⁶⁵, O, and S; and R⁶⁵ is independently hydrogen, C₁-C₅ alkyl, C₃-C₁₀ cycloalkyl, 4-10 membered heterocyclyl, C₆-C₁₀ aryl, and 5-10 membered heteroaryl.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₅ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₅ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₈ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Nitrogen-containing heterocyclyl” group means a 4- to 7-membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

5]“Acyl” refers to a radical —C(O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(O)—C₁-C₈ alkyl, —C(O)—(CH₂)ₜ(C₆-C₁₀ aryl), —C(O)—(CH₂)ₜ(5-10 membered heteroaryl), —C(O)—(CH₂)ₜ(C₃-C₁₀ cycloalkyl), and —C(O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Alkoxy” refers to the group —OR²⁹ where R⁷⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe₂.

“Amino” refers to the radical —NH₂.

“Oxo group” refers to —C(═O)—.

“Substituted amino” refers to an amino group of the formula —N(R³⁸)₂ wherein R³⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, C₁-C₈ alkyl, C₃-C₈ alkenyl, C₃-C₅ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

“Carboxy” refers to the radical —C(O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Haloalkyl” refers to an alkyl radical in which the alkyl group is substituted with one or more halogens. Typical haloalkyl groups include, but are not limited to, trifluoromethyl, difluoromethyl, fluoromethyl, chloromethyl, dichloromethyl, dibromoethyl, tribromomethyl, tetrafluoroethyl, and the like.

“Hydroxy” refers to the radical —OH.

“Nitro” refers to the radical —NO₂.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂—N(R^{bb})₃⁺X⁻, —N(OR^{cc})R^{bb}, —SH, —SR^{aa}, SSR^{cc}, —C(═O)R^{aa}, —CO₂H, —CHO, —C(OR^{cc})₂, CO₂R^{aa}, OC(═O)R^{aa}, OCO₂R^{aa}, —C(═O)N(R^{bb})₂, —OC(═O)N(R^{bb})₂, —NR^{bb}C(═O)R^{aa} —NRCO₂R^{aa}, —NR^{bb}OC(═O)N(R^{bb})₂, —C(═NR^{bb})R₂, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, -SO₂N(R^{bb})₂, —SO₂R^{aa}, —SO₂OR^{aa}, —OSO₂R^{bb}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂ NR^{bb}P(═O)(NR^{bb})₂, —P(R^{cc})₂, —P(R^{cc})₃, —OP(R^{cc})₂, OP(R^{cc})₃, —B(R^{aa})₂, —B(OR^{cc})₂, —BR^{aa}(OR^{cc}), C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{b})₂, ═NNR^{bb}C(═O)R^{aa}, —NNR^{bb}C(═O)OR^{aa}, —NNR^{bb}S(═O)₂R^{aa}, ═NR^{bb}, or ═NOR^{cc};
each instance of R^{aa} is, independently, selected from C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ haloalkyl, C₂₋₁₀ alkenyl, C₂₋₁₀ alkynyl, C₃-10 carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X—, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R)₂, —C(═NR^{ff})OR^{ee}, —OC(═NR^{ee})R^{ee}, —OC(═NR^{ff})OR^{ee}, —C(═NR^{ee})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ff}, -So₂OR^{ee}, —OSO₂R^{ee}, —S(═O)R^{ee} —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, P(═O)(R^{ee})₂, —OP(═O)(R^{ee})₂—OP(═O)(OR^{ee})₂, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{f} is, independently, selected from hydrogen, C₁₋₆-alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{f} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁻X⁻, —NH(C₁₋₆ alkyl)₂+X⁻, —NH₂(C₁₋₆ alkyl)+X⁻, -NH₃+X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂₋₆ alkenyl, C₂₋₆ alkynyl, C₃₋₁₀ carbocyclyl, C₆-10 aryl, 3-10 membered heterocyclyl, 5-10 membered heteroaryl; or two geminal R⁹⁹ substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

These and other exemplary substituents are described in more detail in the Detailed Description, and Claims. The invention is not intended to be limited in any manner by the above exemplary listing of substituents.

### Other Definitions

As used herein, the term “modulation” refers to the inhibition or potentiation of GABAA receptor function. A “modulator” (e.g., a modulator compound) may be, for example, an agonist, partial agonist, antagonist, or partial antagonist of the GABA_{A} receptor.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

The term “prodrug” is intended to encompass therapeutically inactive compounds that, under physiological conditions, are converted into the therapeutically active agents of the present invention. One method for making a prodrug is to design selected moieties that are hydrolyzed or cleaved at a targeted in vivo site of action under physiological conditions to reveal the desired molecule which then produces its therapeutic effect. In certain embodiments, the prodrug is converted by an enzymatic activity of the subject.

In an alternate embodiment, the present invention provides prodrugs of compound of Formulae (1-I), (2-I) or (3-I), wherein the prodrug includes a cleavable moiety on the C3 hydroxy as depicted in Formulae (1-I), (2-I) or (3-I).

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human (“human subject”). In certain embodiments, the subject is a non-human animal.

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{aa})₂, —P(R^{cc})₃—P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), 2-methoxyethoxymethyl (MEM), benzyl (Bn), triisopropylsilyl (TIPS), t-butyldimethylsilyl (TBDMS), i-butylmethoxyphenylsilyl (TBMPS), methanesulfonate (mesylate), and tosylate (Ts).

In certain embodiments, the substituent present on an sulfur atom is an sulfur protecting group (also referred to as a thiol protecting group). Sulfur protecting groups include, but are not limited to, —R^{aa}, —N(R^{U}b)₂, —C(═O)SR^{aa}, —C(═O)R^{aa}, —CO₂R, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂—P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the substituent present on a nitrogen atom is an amino protecting group (also referred to herein as a nitrogen protecting group). Amino protecting groups include, but are not limited to, —OH, —OR^{aa}, —N(R^{cc})₂, —C(═O)R^{aa}, —C(═O)OR^{aa}, —C(═O)N(R^{cc})₂, —S(═O)₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl, C₂₋₁₀ alkenyl, C₂₋₁₀ alkynyl, C₃₋₁₀ carbocyclyl, 3-14-membered heterocyclyl, C₆₋₁₄ aryl, and 5-14-membered heteroaryl groups, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined herein. Amino protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary amino protecting groups include, but are not limited to amide groups (e.g., —C(═O)R^{aa}), which include, but are not limited to, formamide and acetamide; carbamate groups (e.g., —C(═O)OR^{aa}), which include, but are not limited to, 9-fluorenylmethyl carbamate (Fmoc), t-butyl carbamate (BOC), and benzyl carbamate (Cbz); sulfonamide groups (e.g., —S(═O)₂R^{aa}), which include, but are not limited to, p-toluenesulfonamide (Ts), methanesulfonamide (Ms), and N-[2-(trimethylsilyl)ethoxy]methylamine (SEM).

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (also, “therapeutic treatment”).

“Prophylactic treatment” contemplates an action that occurs before a subject begins to suffer from the specified disease, disorder or condition.

In general, the “effective amount” of a compound refers to an amount sufficient to elicit the desired biological response, e.g., to treat a CNS-related disorder, is sufficient to induce anesthesia or sedation. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternate embodiment, the present invention contemplates administration of the compounds of the present invention or a pharmaceutically acceptable salt or a pharmaceutically acceptable composition thereof, as a prophylactic before a subject begins to suffer from the specified disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### Compounds

It should be appreciated that formulas described herein may reference particular carbon atoms, such as C17, C3, C19, etc. These references are based on the position of carbon atoms according to steroid nomenclature known and used in the industry, as shown below:

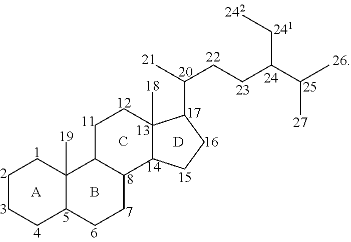

For example, C17 refers to the carbon at position 17 and C3 refers to the carbon at position 3.

In one aspect, provided herein is a compound of Formula (1-I):

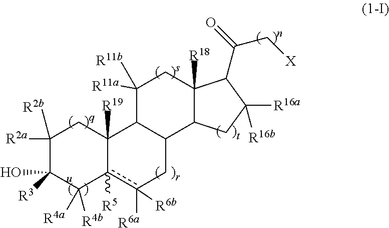

or a pharmaceutically acceptable salt thereof;
wherein:
q is independently 0, 1, 2, or 3;
r is independently 0, 1 or 2;
s is independently 0, 1 or 2;
t is independently 0, 1, 2 or 3;
n is independently 1 or 2;
u is independently 1 or 2;
X is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R^{A2}, OS(═O)₂OR^{A1}, SS(═O)₂R^{A2}, S—S(═O)₂OR^{A1}, S(═O)R^{A2}, SO₂R^{A2}, or S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, or a nitrogen protecting group when attached to a nitrogen atom; and each instance of R^{A2} is independently substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl, or when [symbol] is a double bond, R⁵ and one of R^{6a} or R^{6b} is absent;
R¹⁹ is hydrogen or substituted or unsubstituted alkyl;
R¹⁸ is substituted or unsubstituted alkyl, substituted or unsubstituted alkenyl, or substituted or unsubstituted alkynyl;
R³ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each of R^{6a} and R^{6b} is independently hydrogen, halogen, cyano, hydroxyl, substituted or unsubstituted alkyl, substituted or unsubstituted alkenyl, or substituted or unsubstituted alkynyl; or R^{6a} and R^{6b} are joined to form an oxo (═O) group; and each of R^{2a}, R^{2b}, R^{4a}, R^{4b}, R^{11a}, R^{11b}, R^{16a} or R^{16b} is independently hydrogen, halogen, cyano, hydroxyl, substituted or unsubstituted alkyl, substituted or unsubstituted alkenyl, substituted or unsubstituted heterocyclyl, substituted or unsubstituted alkynyl, —OR^{D1}, —OC(═O)R^{D1}, —NH₂, —N(R^{D1})₂, or —NR^{D1}C(═O)R^{D1}, wherein each instance of R¹ is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{2a} and R^{2b}, or R^{4a} and R^{4b}, or R^{11a} and R^{11b} or R^{16a} and R^{16b} are joined to form an oxo (═O) group; provided that:
   q, s, r, u, and t are not simultaneously 1.

In some embodiments of a compound of Formula (1-I) when t is 0, 2, or 3 then q, u, s, and r are not simultaneously 1. In some embodiments of a compound of Formula (1-I) when q is 0 or 2 and u is 1, then t, s, and r are not simultaneously 1. In some embodiments of a compound of Formula (1-I) when u is 2 and q is 1, then t, s, and r are not simultaneously 1. In some embodiments of a compound of Formula (1-I) when r is 0 or 2, then q, u, s, and t are not simultaneously 1. In some embodiments of a compound of Formula (1-I) when s is 0 or 2, then q, u, r, and t are not simultaneously 1.

In one embodiment, provided herein is a compound of Formula (1-V-a) or Formula (1-V-b):

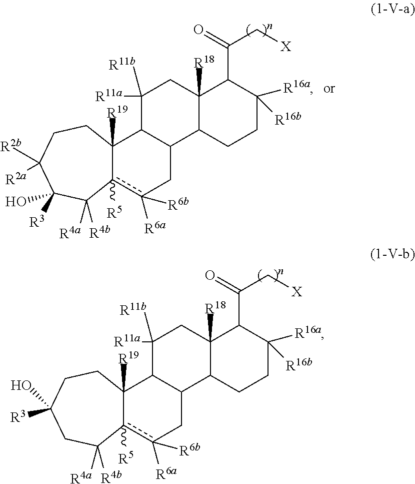

or a pharmaceutically acceptable salt thereof;
wherein:
n is 1 or 2;
X is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A})₂, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}; wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom; and each instance of R^{A2} is independently substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl, or when [symbol] is a double bond, R⁵ is absent;
R¹⁹ is hydrogen or substituted or unsubstituted alkyl;
R¹⁸ is substituted or unsubstituted alkyl, substituted or unsubstituted alkenyl, or substituted or unsubstituted alkynyl;
R³ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each of R^{6a} and R^{6b} is independently hydrogen, halogen, cyano, hydroxyl, substituted or unsubstituted alkyl, substituted or unsubstituted alkenyl, or substituted or unsubstituted alkynyl; or R^{6a} and R^{6b} are joined to form an oxo (═O) group; and
each of R^{2a}, R^{2b}, R^{4a}, R^{4b}, R^{11a}, R^{11b}, R^{16a}, or R^{16b} is independently hydrogen, halogen, cyano, hydroxyl, substituted or unsubstituted alkyl, substituted or unsubstituted alkenyl, substituted or unsubstituted heterocyclyl, substituted or unsubstituted alkynyl, —OR^{D1}, —OC(═O)R^{D1}, —NH₂, —N(R^{D1})₂, or —NR^{D1}C(═O)R^{D1}; wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, or a nitrogen protecting group when attached to a nitrogen atom; or two R^{D1} groups are joined to form an substituted or unsubstituted heterocyclic ring; or any one of R^{2a} and R^{2b}, or R^{4a} and R^{4b}, or R^{11a} and R^{11b}, or R^{16a} and R^{16b} are joined to form an oxo (═O) group.

In some embodiments, [symbol] is a single bond. In another embodiment, [symbol] is a double bond.

In some embodiments, each of R^{2a}, R^{2b}, R^{4a}, R^{4b}, R^{6a}, R^{6b}, R^{11a}, R^{11b}, R^{16a}, or R^{16b} is independently hydrogen, substituted or unsubstituted alkyl, substituted or unsubstituted alkenyl, —OH, or —OR^{D1}; or any one of R^{2a} and R^{2b}, or R^{4a} and R^{4b}, or R^{11a} and R^{11b}, or R^{16a} and R^{16b} are joined to form an oxo (═O) group; wherein each alkyl is optionally substituted with a substitutent selected from halo, —OH, or —OR^{D1}; and wherein each R^{D1} is independently hydrogen, haloalkyl, or unsubstituted alkyl.

In some embodiments, R^{2a}, R^{2b}, R^{4a}, R^{4b}, R^{6a}, R^{6b}, R^{11a}, R^{11b}, R^{16a}, or R^{16b} are hydrogen.

In an aspect, provided herein is a compound of Formula 2-I:

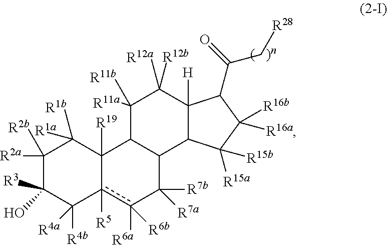

or a pharmaceutically acceptable salt thereof;
wherein:
[symbol] represents a single or double bond, provided if a double bond is present, then R⁵ and one of R^{6a} or R^{6b} are absent;
R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or substituted or unsubstituted methyl, or when [symbol] is a double bond, R⁵ is absent;
each of R^{6a} and R^{6b} is independently hydrogen, halogen, cyano, hydroxyl, substituted or unsubstituted alkyl, substituted or unsubstituted alkenyl, or substituted or unsubstituted alkynyl, or R^{6a} and R^{6b} are joined to form an oxo (═O) group; each of R^{1a}, R^{1b}, R^{2a}, R^{2b}, R^{4a}, R^{4b}, R^{7a}, R^{7b}, R^{11a}, R^{11b}, R^{12a}, R^{12b}, R^{15a}, and R^{15b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, -OR^{D1}, —OC(═O)R^{D1}, —NH₂, —N(R^{D1})₂, or -NRDC(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{11b}, R^{12a} and R^{12b}, and R^{15a} and R^{15b} are joined to form an oxo (═O) group;
each of R^{16a} and R^{16b} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —N(R^{A1}) —CN(R^{A1})₂, —C(O)R^{A1}, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R¹⁹ is hydrogen, substituted or unsubstituted alkyl, substituted or unsubstituted alkenyl, or substituted or unsubstituted alkynyl;
R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
n is 1, 2, or 3; and
with the proviso that the compound is not:

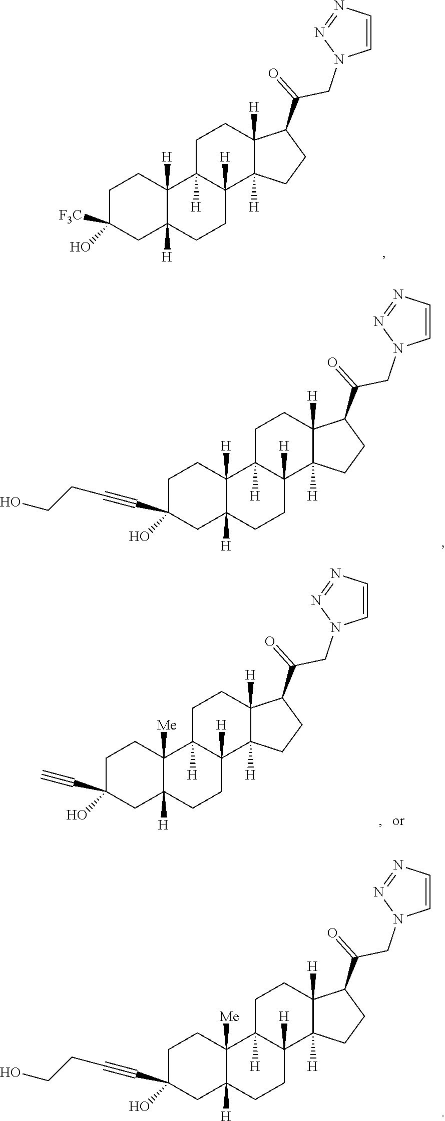

In another aspect, provided herein is a method of treating a CNS-related disorder in a subject in need thereof, comprising administering to the subject a compound of Formula 2-I:

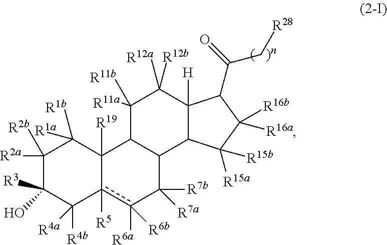

or a pharmaceutically acceptable salt thereof;
wherein:
[symbol] represents a single or double bond, provided if a double bond is present, then R⁵ and one of R^{6a} or R^{6b} are absent;
R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or substituted or unsubstituted methyl, or when [symbol] is a double bond, R⁵ is absent;
each of R^{6a} and R^{6b} is independently hydrogen, halogen, cyano, hydroxyl, substituted or unsubstituted alkyl, substituted or unsubstituted alkenyl, or substituted or unsubstituted alkynyl, or R^{6a} and R^{6b} are joined to form an oxo (═O) group;
each of R^{1a}, R^{1b}, R^{2a}, R^{2b}, R^{4a}, R^{4b}, R^{7a}, R^{7b}, R^{11a}, R^{11b}, R^{12a}, R^{12b}, R^{15a}, and R^{15b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NRPC(═O)R^{D1} wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{11b}, R^{12a} and R^{12b}, and R^{15a} and R^{15b} are joined to form an oxo (═O) group; each of R^{16a} and R^{16b} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —N(R^{A1}) —CN(R^{A1})₂, —C(O)R^{A1}, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, OS(═O)₂R^{A2}, —OS(═O)₂OR^{A1}, S S(═O)₂R^{A2}, —S—S(═O)₂OR^{A1}, —S(═O)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R¹⁹ is hydrogen, substituted or unsubstituted alkyl, substituted or unsubstituted alkenyl, or substituted or unsubstituted alkynyl;
R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring; and
n is 1, 2, or 3.

In some embodiments, the compound of Formula 2-I is a compound of Formula 2-Ia or Formula 2-Ib:

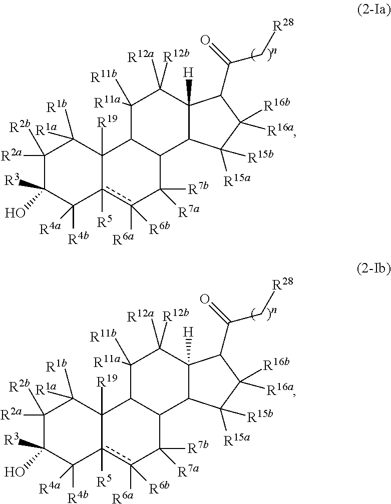

or a pharmaceutically acceptable salt thereof.

In some embodiments, the compound of Formula 2-I is a compound of Formula 2-Iaa or Formula 2-Iab:

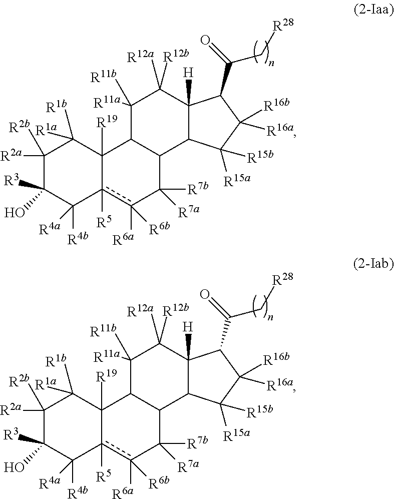

or a pharmaceutically acceptable salt thereof.

In some embodiments, provided herein is a compound of Formula 2-II:

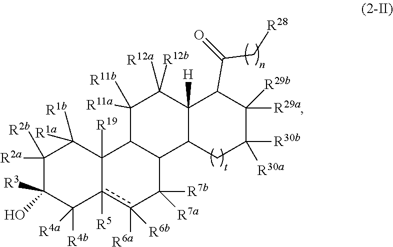

or a pharmaceutically acceptable salt thereof;
wherein:
t is 1 or 2;
[symbol] represents a single or double bond, provided if a double bond is present, then R⁵ and one of R^{6a} or R^{6b} are absent; R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; R⁵ is hydrogen or substituted or unsubstituted methyl, or when [symbol] is a double bond, R⁵ is absent; each of R^{6a} and R^{6b} is independently hydrogen, halogen, cyano, hydroxyl, substituted or unsubstituted alkyl, substituted or unsubstituted alkenyl, or substituted or unsubstituted alkynyl, or R^{6a} and R^{6b} are joined to form an oxo (═O) group; each of R^{1a}, R^{1b}, R^{2a}, R^{2b}, R^{4a} R^{4b}, R^{7a}, R^{7b}, R^{11a}, R^{11b}, R^{12a}, R^{12b}, R^{30a}, and R^{30b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1} wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{11b}, R^{12a} and R^{12b}, and R^{30a} and R^{30b} are joined to form an oxo (═O) group;
each of R²⁹ and R^{29b} is each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1} —N(R^{A1})₂—N(R^{A1}), —CN(R^{A1})₂, —C(O)R^{A1}, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, SC(═O)SR^{A1}, —SC(═O)N(R^{A1})₂, NHC(═O)R^{A1}, —NHC(═O)OR^{A1}, —NHC(═O)SR^{A1}, —NHC(═O)N(R^{A1})₂, —OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R¹⁹ is hydrogen, substituted or unsubstituted alkyl, substituted or unsubstituted alkenyl, or substituted or unsubstituted alkynyl;
R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring; and
n is 1, 2, or 3.

In some embodiments, the compound of Formula 2-II is a compound of Formula 2-IIa:

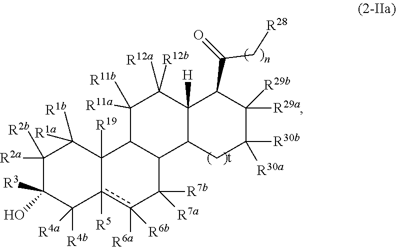

or a pharmaceutically acceptable salt thereof.

In some embodiments, provided herein is a compound of Formula 2-III:

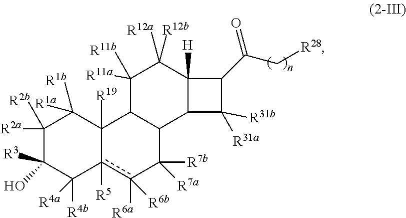

or a pharmaceutically acceptable salt thereof;
wherein:
[symbol] represents a single or double bond, provided if a double bond is present, then R⁵ and one of R^{6a} or R^{6b} are absent;
R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or substituted or unsubstituted methyl, or when [symbol] is a double bond, R⁵ is absent;
each of R^{6a} and R^{6b} is independently hydrogen, halogen, cyano, hydroxyl, substituted or unsubstituted alkyl, substituted or unsubstituted alkenyl, or substituted or unsubstituted alkynyl, or R^{6a} and R^{6b} are joined to form an oxo (═O) group;
each of R^{1a}, R^{1b}, R^{2a}, R^{2b}, R^{4a}, R^{4b}, R^{7a}, R^{7b}, R^{11a}, R^{11b}, R^{12a}, and R^{12b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{11b}, R^{12a} and R^{12b} are joined to form an oxo (═O) group;
each of R^{31a} and R^{31b} is each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —N(R^{A1}), —CN(R^{A1})₂, —C(O)R^{A1}, —OC(═O)R^{A1}, —OC(═O)OR^{A1}, —OC(═O)SR^{A1}, OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A}], —NHC(═O)OR^{A1}, —NHC(═O)SR^{A1}, —NHC(═O)N(R^{A1})₂, —OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R¹⁹ is hydrogen, substituted or unsubstituted alkyl, substituted or unsubstituted alkenyl, or substituted or unsubstituted alkynyl;
R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring; and n is 1, 2, or 3.

In some embodiments, the compound of Formula 2-III is a compound of Formula 2-IIIa:

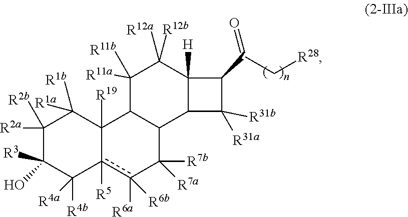

or a pharmaceutically acceptable salt thereof.

In some embodiments, provided herein is a compound of Formula 2-IVa or Formula 2-IVb:

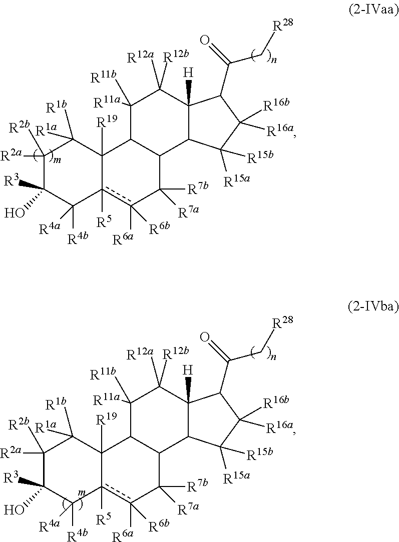

or a pharmaceutically acceptable salt thereof;
wherein:
[symbol] represents a single or double bond, provided if a double bond is present, then R⁵ and one of R^{6a} or R^{6b} are absent;
R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or substituted or unsubstituted methyl, or when [symbol] is a double bond, R⁵ is absent;
each of R^{6a} and R^{6b} is independently hydrogen, halogen, cyano, hydroxyl, substituted or unsubstituted alkyl, substituted or unsubstituted alkenyl, or substituted or unsubstituted alkynyl, or R^{6a} and R^{6b} are joined to form an oxo (═O) group;
each of R^{1a}, R^{1b}, R^{2a}, R^{2b}, R^{4a}, R^{4b}, R^{7a}, R^{7b}, R^{11a}, R^{11b}, R^{12a}, R^{12b}, R^{15a}, and R^{15b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{11b}, R^{2a} and R^{12b}, and R^{15a} and R^{15b} are joined to form an oxo (═O) group;
each of R^{16a} and R^{16b} is each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —N(R^{A1}), —CN(R^{A1})₂, —C(O)R^{A1}, —OC(═O)R^{A1}, —OC(═O)OR^{A1}, —OC(═O)SR^{A1}, OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R², —OS(═O)₂OR^{A1}, —S—S(═O)₂R^{A2}, —S—S(═O)₂OR^{A1}, —S(═O)R^{A2}, SO₂R^{A2}, or —S(═O)₂OR^{A1} wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R¹⁹ is hydrogen, substituted or unsubstituted alkyl, substituted or unsubstituted alkenyl, or substituted or unsubstituted alkynyl;
R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂ —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
n is 1, 2, or 3; and
m is 2 or 3.

In some embodiments, the compound of Formula 2-IV is a compound of Formula 2-IVaa or Formula 2-IVba:

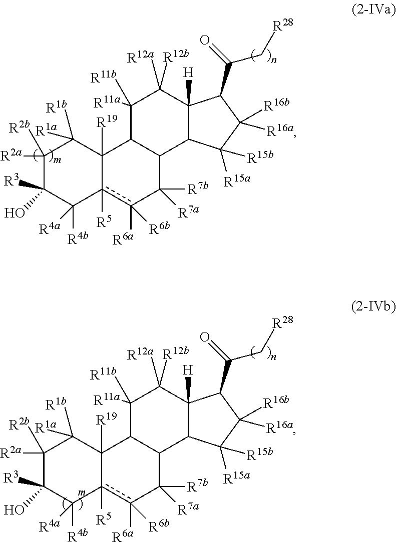

or a pharmaceutically acceptable salt thereof.

In some embodiments, provided herein is a compound of Formula 2-V:

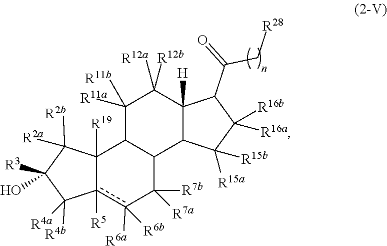

or a pharmaceutically acceptable salt thereof;
wherein:
[symbol] represents a single or double bond, provided if a double bond is present, then R⁵ and one of R^{6a} or R^{6b} are absent; R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or substituted or unsubstituted methyl, or when [symbol] is a double bond, R⁵ is absent;
each of R^{6a} and R^{6b} is independently hydrogen, halogen, cyano, hydroxyl, substituted or unsubstituted alkyl, substituted or unsubstituted alkenyl, or substituted or unsubstituted alkynyl, or R^{6a} and R^{6b} are joined to form an oxo (═O) group; each of R^{2a}, R^{2b}, R^{4a}, R^{4b}, R^{7a}, R^{7b}, R^{11a}, R^{11b}, R^{12a} R^{12b} R^{15a} and R^{15b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{11b}, R^{12a} and R^{12b}, and R^{15a} and R^{15b} are joined to form an oxo (═O) group;
each of R^{16a} and R^{16b} is each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —N(R^{A1}), —CN(R^{A1})₂, —C(O)R^{A1}, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R¹⁹ is hydrogen, substituted or unsubstituted alkyl, substituted or unsubstituted alkenyl, or substituted or unsubstituted alkynyl;
R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring; and
n is 1, 2, or 3.

In some embodiments, the compound of Formula 2-V is a compound of Formula 2-Va:

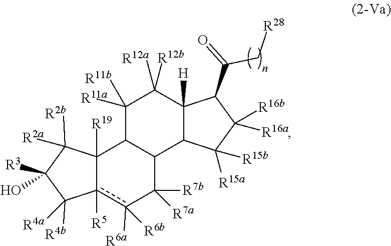

or a pharmaceutically acceptable salt thereof.

In some embodiments, provided herein is a compound of Formula 2-VI:

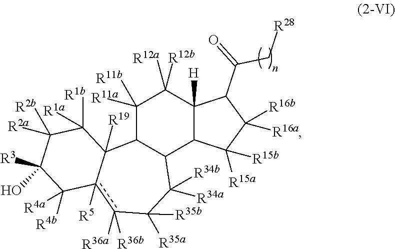

or a pharmaceutically acceptable salt thereof;
wherein:
[symbol] represents a single or double bond, provided if a double bond is present, then R⁵ and one of R^{36a} or R^{36b} are absent;
R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or substituted or unsubstituted methyl, or when [symbol] is a double bond, R⁵ is absent;
each of R^{36a} and R^{36b} is independently hydrogen, halogen, cyano, hydroxyl, substituted or unsubstituted alkyl, substituted or unsubstituted alkenyl, or substituted or unsubstituted alkynyl, or R^{36a} and R^{36b} are joined to form an oxo (═O) group; each of R^{1a}, R^{1b}, R^{2a}, R^{2b}, R^{4a}, R^{4b}, R^{11a}, R^{11b}, R^{12a}, R^{12b}, R^{15a}, R^{15a}, R^{34a}, R^{34b}, R^{35a} and R^{35b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{11b}, R^{12a} and R^{12b}, and R^{15a} and R^{15b} are joined to form an oxo (═O) group;
each of R^{16a} and R^{16b} is each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1} —N(R^{A1})₂, —N(R^{A1}), —CN(R^{A1})₂, —C(O)R^{A1}, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R¹⁹ is hydrogen, substituted or unsubstituted alkyl, substituted or unsubstituted alkenyl, or substituted or unsubstituted alkynyl;
R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, -OR^{A1}, —N(R^{A})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring; and n is 1, 2, or 3.

In some embodiments, the compound of Formula 2-VT is a compound of Formula 2-VIa:

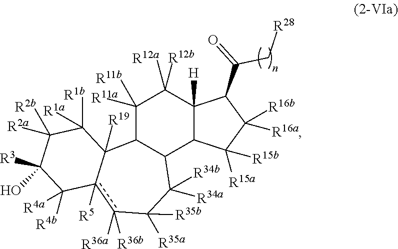

or a pharmaceutically acceptable salt thereof.

In some embodiments, provided herein is a compound of Formula 2-VII:

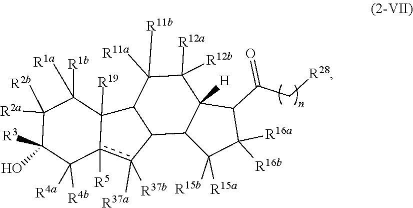

or a pharmaceutically acceptable salt thereof;
wherein:
[symbol] represents a single or double bond, provided if a double bond is present, then R⁵ and one of R^{36a} or R^{36b} are absent;
R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or substituted or unsubstituted methyl, or when [symbol] is a double bond, R⁵ is absent;
each of R^{37a} and R^{17b} is independently hydrogen, halogen, cyano, hydroxyl, substituted or unsubstituted alkyl, substituted or unsubstituted alkenyl, or substituted or unsubstituted alkynyl, or R^{37a} and R^{37b} are joined to form an oxo (═O) group;
each of R^{1a}, R^{1b}, R^{2a}, R^{2b}, R^{4a}, R^{4b}, R^{11a}, R^{11b}, R^{12a}, R^{12b}, R^{15a}, and R^{15b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{11b}, R^{12a} and R^{12b}, and R^{15a} and R^{15b} are joined to form an oxo (═O) group;
each of R^{16a} and R^{16b} is each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1} —N(R^{A1})₂, —N(R^{A1}), —CN(R^{A1})₂, —C(O)R^{A1}, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R¹⁹ is hydrogen, substituted or unsubstituted alkyl, substituted or unsubstituted alkenyl, or substituted or unsubstituted alkynyl;
R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring; and
n is 1, 2, or 3.

In some embodiments, the compound of Formula 2-VII is a compound of Formula 2-VIIa:

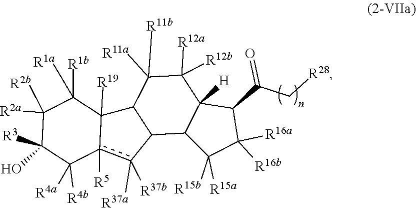

or a pharmaceutically acceptable salt thereof.

In an aspect, provided herein is a compound of Formula 3-T

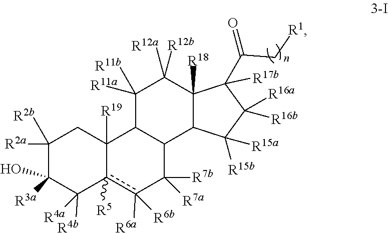

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂—OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{12a}, R^{12b} or R^{4b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent; each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group; each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁸ is substituted or unsubstituted alkyl;
R¹⁹ is substituted or unsubstituted C₃-C₆ carbocyclyl or substituted or unsubstituted aryl; and n is 0, 1 or 2.

In an aspect, provided herein is a compound of Formula 3-Ix

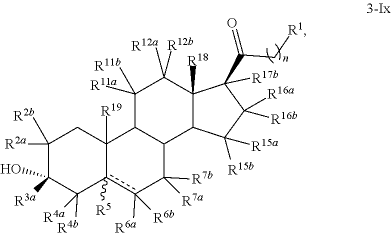

or a pharmaceutically acceptable salt thereof; wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent;
each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁸ is substituted or unsubstituted alkyl;
R¹⁹ is substituted or unsubstituted C₃-C₆ carbocyclyl or substituted or unsubstituted aryl; and
n is 0, 1 or 2.

In some aspects, the compound of Formula I is a compound of Formula 3-IIa or Formula IIb:

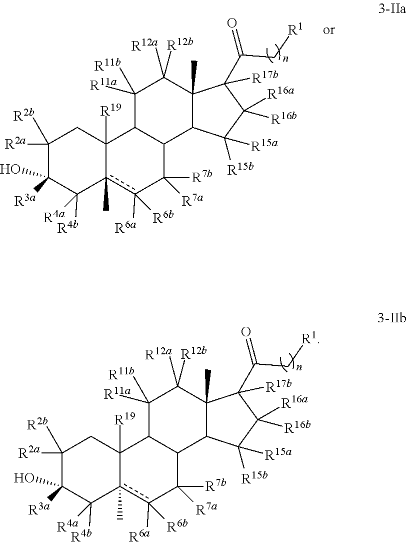

In another embodiment, the compound is a compound of Formula 3-III:

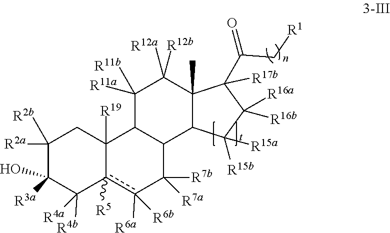

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{At} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R⁷, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent;
each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{11a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted C₃-C₆ carbocyclyl or substituted or unsubstituted aryl;
n is 0, 1 or 2 and
t is 2 or 3.

In another embodiment, the compound is a compound of Formula 3-IIx:

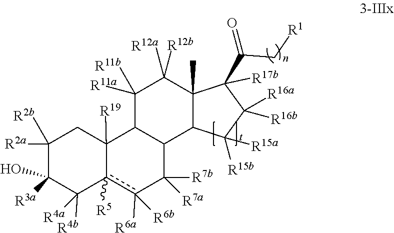

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{2a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂—OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent;
each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted C₃-C₆ carbocyclyl or substituted or unsubstituted aryl;
n is 0, 1 or 2 and
t is 2 or 3.

In some further embodiments, the compound is a compound of Formula 3-IV:

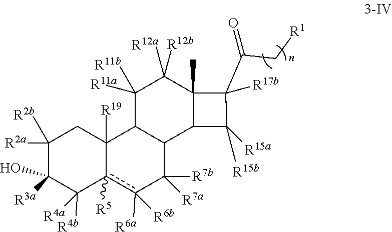

or a pharmaceutically acceptable salt thereof;
wherein:
[symbol] represents a single or double bond, provided if a double bond is present, then one of R^{6a} or R^{1b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂—SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent;
each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{15a} and R^{15b} are joined to form an oxo (═O) group; each of R^{15a} and R^{15b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted C₃-C₆ carbocyclyl or substituted or unsubstituted aryl; and
n is 0, 1 or 2.

In other embodiments, the compound is of Formula 3-Va or Formula 3-Vb:

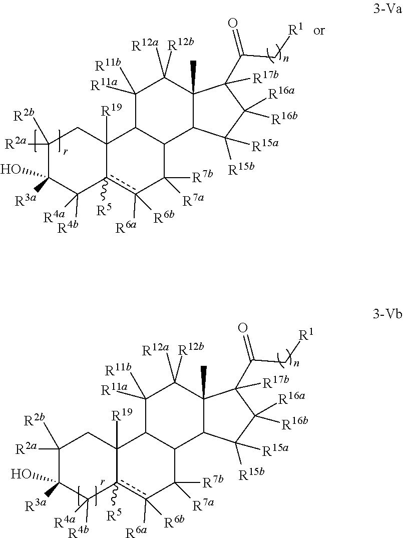

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, -OR^{A1}, —N(R^{A1})₂, —SR^{A1}, —C(═O)R^{A1}, —C(═O)OR^{A1}, —C(═O)SR^{A1}, —C(═O)N(R^{A})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂—OC(═O)SR^{A1}, —OS(═O)₂R^{A1}, —OS(═O)₂OR^{A1}, —OS(O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent;
each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted C₃-C₆ carbocyclyl or substituted or unsubstituted aryl;
n is 0, 1 or 2; and
r is 2 or 3.

In other embodiments, the compound is of Formula 3-Vax or Formula 3-Vbx:

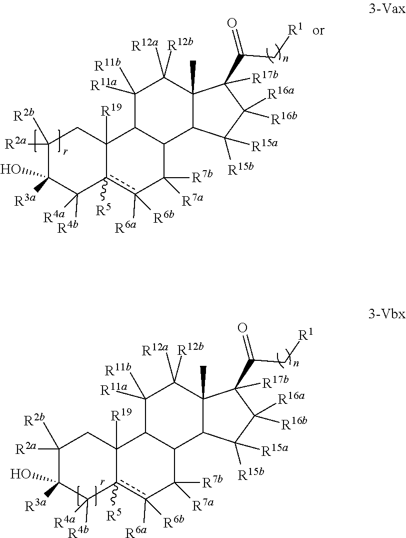

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, -OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(RAJ)₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent; [symbol]
each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂ —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted C₃-C₆ carbocyclyl or substituted or unsubstituted aryl;
n is 0, 1 or 2; and
r is 2 or 3.

In other embodiments, the compound is of Formula 3-VI:

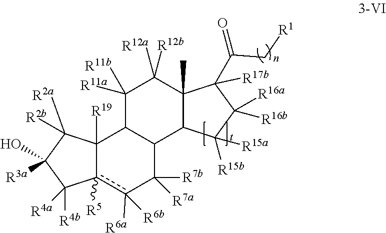

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, -OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent; each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group; each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂₀R^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted C₃-C₆ carbocyclyl or substituted or unsubstituted aryl;
n is 0, 1 or 2.

In some embodiments, the compounds is of Formula 3-VII:

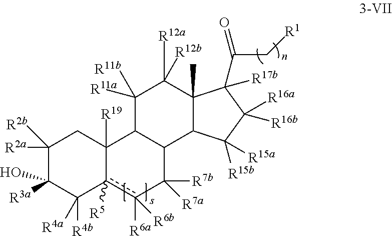

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂—OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent;
each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted C₃-C₆ carbocyclyl or substituted or unsubstituted aryl;
n is 0, 1 or 2; and
s is 2.

In some embodiments, the compound is of Formula 3-VIII:

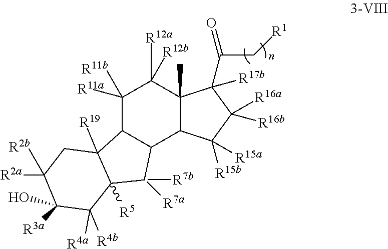

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{12a}, R^{12a} or R^{12b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent;
each of R^{15a}, R^{15b}, R^{6a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂—S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted C₃-C₆ carbocyclyl or substituted or unsubstituted aryl; and
n is 0, 1 or 2.

In other embodiments, the compound is of Formula 3-IX:

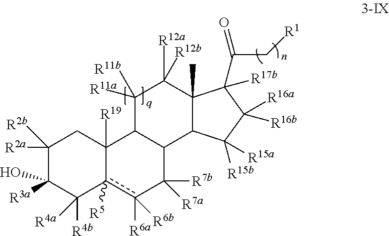

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{At} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R⁷, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent;
each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{11a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted C₃-C₆ carbocyclyl or substituted or unsubstituted aryl;
n is 0, 1 or 2; and
q is 2.

In some embodiments, the compound is of Formula 3-Ia:

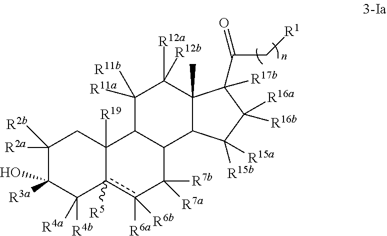

or a pharmaceutically acceptable salt thereof;
wherein:
[symbol] represents a single or double bond, provided if a double bond is present, then one of R^{6a} or R^{b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R¹², R^{12b} or R^{1b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent;
each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{15a}, R^{15a}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3} —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R^{1a} is substituted or unsubstituted alkyl;
R¹⁹ is substituted or unsubstituted heterocyclyl or substituted or unsubstituted heteroaryl; and
n is 0, 1 or 2.

In some embodiments, the compound is a compound of Formula 3-IIaa or Formula 3-IIba:

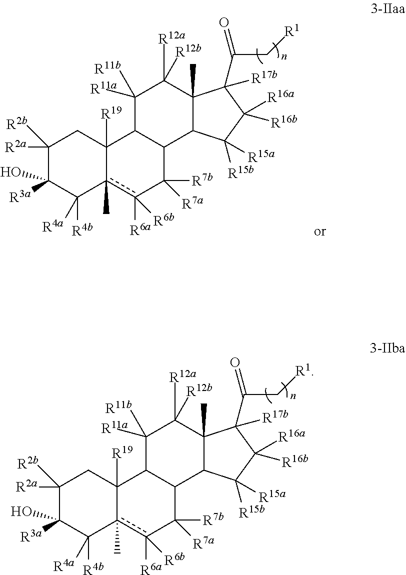

In some embodiments, the compound is of Formula 3-IIIa:

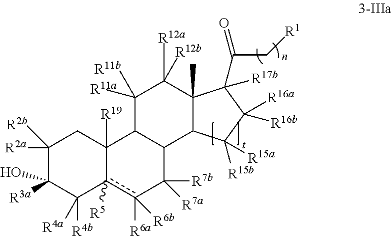

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R¹², R^{12b} or R^{1b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent;
each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{15a}, R^{15a}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3} —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted heterocyclyl or substituted or unsubstituted heteroaryl;
n is 0, 1 or 2 and
t is 2 or 3.

In some embodiments, the compound is of Formula 3-IVa:

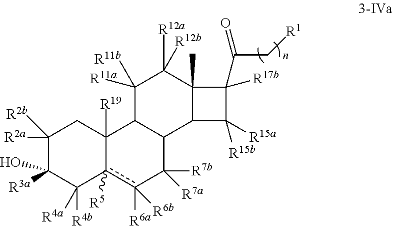

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, -OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{2a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent; each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{15a} and R^{15b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted heterocyclyl or substituted or unsubstituted heteroaryl; and
n is 0, 1 or 2.

In some embodiments, the compound is of Formula 3-Vac or Formula 3-Vacc:

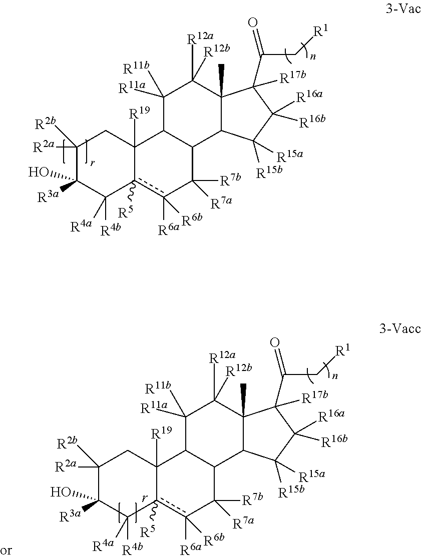

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent;
each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{5a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C}3)₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted heterocyclyl or substituted or unsubstituted heteroaryl;
n is 0, 1 or 2; and
r is 2 or 3.

In some embodiments, the compound is of Formula 3-VIac:

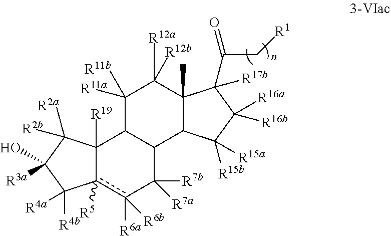

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{7T}, R^{7b}, R^{11a}, R^{11b}, R¹², R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent;
each of R⁶ and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂—S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted heterocyclyl or substituted or unsubstituted heteroaryl;
n is 0, 1 or 2.

In some embodiments, the compound is of Formula 3-VIIac:

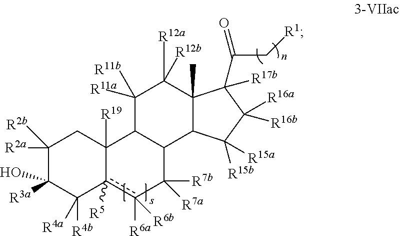

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{At} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent; each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{11a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂₀R^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted heterocyclyl or substituted or unsubstituted heteroaryl;
n is 0, 1 or 2; and
s is 2.

In some embodiments, the compound is of Formula 3-VIIIac:

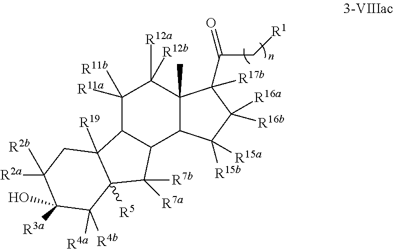

or a pharmaceutically acceptable salt thereof;
wherein:
[symbol] represents a single or double bond, provided if a double bond is present, then one of R^{6a} or R^{6b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂—OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent; each of R^{1a}, R^{15b}, R^{6a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C}3)₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted heterocyclyl or substituted or unsubstituted heteroaryl; and
n is 0, 1 or 2.

In some embodiments, the compound is of Formula 3-IXac:

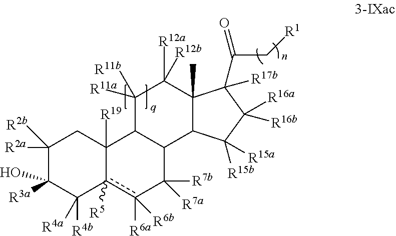

or a pharmaceutically acceptable salt thereof;
wherein:
[symbol] represents a single or double bond, provided if a double bond is present, then one of R^{6a} or R^{b} is absent;
R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R¹², R^{12b} or R^{1b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl; when [symbol] is a double bond, R⁵ is absent; each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3} —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C}3)₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
R¹⁹ is substituted or unsubstituted heterocyclyl or substituted or unsubstituted heteroaryl;
n is 0, 1 or 2; and
q is 2.

In some embodiments, R¹⁹ is not

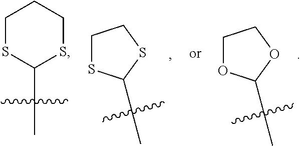

In some embodiments, the compound of Formula (1-I) is a compound is of Formula (1-II), Formula (1-III), Formula (1-IV), Formula (1-V—), Formula (1-VI), Formula (1-VII), Formula (1-VIII), Formula (1-IX), Formula (1-X), Formula (1-XI), Formula (1-XII), Formula (1-XIII), or Formula (1-XIV).

Groups R^{2a} and R^{2b}

In some embodiments R² and R^{2b} is each independently hydrogen, halogen, cyano, hydroxyl, substituted or unsubstituted alkyl, substituted or unsubstituted alkenyl, substituted or unsubstituted heterocyclyl, substituted or unsubstituted alkynyl, —OR^{D1}, —OC(═O)R^{D1}, —NH₂, —N(R^{D1})₂, or —NR^{D1}C(═O)R^{D1}; wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

For example, R^{2a} and R^{2b} is each independently hydrogen, halogen, cyano, hydroxyl, substituted or unsubstituted alkyl, —OR^{D1}, or —OC(═O)R^{D1}; wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In another example, R^{2a} and R^{2b} is each independently hydrogen, halogen, cyano, hydroxyl, substituted or unsubstituted alkyl, or —OR^{D1}; wherein each instance of R^{D1} is independently hydrogen, or substituted or unsubstituted alkyl.

In another example, R^{2a} and R^{2b} are each independently hydrogen.

In one embodiment, R^{2a} and R^{2b} are both hydrogen.

Groups R^{4a} and R^{4b}

In some embodiments, R^{4a} and R^{4b} is each independently hydrogen, halogen, cyano, hydroxyl, substituted or unsubstituted alkyl, substituted or unsubstituted alkenyl, substituted or unsubstituted heterocyclyl, substituted or unsubstituted alkynyl, —OR^{D1}, —OC(═O)R^{D1}, —NH₂, —N(R^{D1})₂, or —NR^{D1}C(═O)R^{D1}; wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

In another embodiment, R^{4a} and R^{4b} is each independently hydrogen, halogen, cyano, hydroxyl, substituted or unsubstituted alkyl, —OR^{D1}, or —OC(═O)R^{D1}; wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In some embodiments, R^{4a} and R^{4b} is each independently hydrogen, halogen, cyano, hydroxyl, substituted or unsubstituted alkyl, or —OR^{D1}; wherein each instance of RD is independently hydrogen, or substituted or unsubstituted alkyl.

In some embodiments, R^{4a} and R^{4b} are each independently hydrogen.

In one embodiment, R^{4a} and R^{4b} are both hydrogen.

In embodiments, each of R^{2a} and R^{2b} is independently hydrogen or substituted or unsubstituted alkyl.

In certain embodiments, each of R^{2a} and R^{2b} is independently hydrogen, C₁-C₆ alkyl, C₁-C₆ haloalkyl, C₁-C₆ alkoxy, C₁-C₆ alkoxyhalo, or —OH.

In some embodiments, each of R^{2a} and R^{2b} is independently —CH₃, —CH₂CH₃, —OH, —OCH₃, or —CH(CH₃)₂.

In some embodiments, both R^{2a} and R^{2b} are hydrogen.

In some embodiments, rR^{2a} and R^{2b} are each independently hydrogen.

Groups R^{6a} and R^{6b}

In some embodiments, R^{6a} and R^{6b} is each independently hydrogen, halogen, cyano, hydroxyl, substituted or unsubstituted alkyl, substituted or unsubstituted alkenyl, substituted or unsubstituted heterocyclyl, substituted or unsubstituted alkynyl, -OR^{D1}, —OC(═O)R^{D1}, —NH₂, —N(R^{D1})₂, or —NR^{D1}C(═O)R^{D1}; wherein each instance of R¹ is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

In one embodiment, R^{6a} and R^{6b} is each independently hydrogen, halogen, cyano, hydroxyl, substituted or unsubstituted alkyl, —OR^{D1}, or —OC(═O)R^{D1}, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In some embodiments, R^{6a} and R^{6b} is each independently hydrogen, halogen, cyano, hydroxyl, substituted or unsubstituted alkyl, or —OR^{D1}; wherein each instance of R^{D1} is independently hydrogen, or substituted or unsubstituted alkyl.

In some embodiments, R^{6a} and R^{6b} are each independently hydrogen.

In one embodiment, R^{6a} and R^{6b} are both hydrogen.

In embodiments, each of R^{6a} and R^{6b} is independently hydrogen, halogen, substituted or unsubstituted alkyl, substituted or unsubstituted alkenyl, or substituted or unsubstituted alkynyl.

In embodiments, both R^{6a} and Reb are halogen.

In some embodiments, both R^{6a} and R^{6b} are alkyl.

In some embodiments, R^{6a} and R^{6b} are joined to form an oxo group.

In embodiments, each of R^{6a} and R^{6b} is independently hydrogen or substituted or unsubstituted alkyl.

In embodiments, each of R^{6a} and R^{6b} is independently hydrogen or substituted alkyl.

In some embodiments, each of R^{6a} and R^{6b} is independently hydrogen or unsubstituted alkyl.

In certain embodiments, R^{6a} is halogen or alkyl and R^{6b} is hydrogen.

In certain embodiments, both R^{6a} and R^{6b} are hydrogen.

In some embodiments, each of R^{6a} and R^{6b} is independently hydrogen.

Group R^{7a} and R^{7b}

In some embodiments, each of R^{7a} and R^{7b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

In some embodiments, each of R^{7a} and R^{7b} is independently hydrogen, halogen, cyano, hydroxyl, substituted or unsubstituted alkyl, substituted or unsubstituted alkenyl, —OR^{D1}, —OC(═O)R^{D1}, —NH₂, or —N(R^{D1})₂, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In embodiments, each of R⁷ and R-h is independently hydrogen, substituted or unsubstituted alkyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In embodiments, each of R^{7a} and R^{7b} is independently hydrogen, C₁-C₆ alkyl, C₁-C₆ haloalkyl, C₁-C₆ alkoxy, C₁-C₆ alkoxyhalo, or —OH.

In some embodiments, each of R^{7a} and R^{7b} is independently —CH₃, —CH₂CH₃, —OH, —OCH₃, or —CH(CH₃)₂.

In some embodiments, each of R^{7a} and R^{7b} is independently hydrogen or substituted or unsubstituted alkyl.

In embodiments, both R^{7a} or R^{7b} are hydrogen.

Groups R^{11a} and R^{11b}

In some embodiments, R^{11a} and R^{11b} is each independently hydrogen, halogen, cyano, hydroxyl, substituted or unsubstituted alkyl, substituted or unsubstituted alkenyl, substituted or unsubstituted heterocyclyl, substituted or unsubstituted alkynyl, —OR^{D1}—OC(═O)R^{D1}, NH₂, —N(R^{D1})₂, or —NR^{D1}C(═O)R^{D1}; wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

In some embodiments, R^{11a} and R^{11b} is each independently hydrogen, halogen, cyano, hydroxyl, substituted or unsubstituted alkyl, —OR^{D1}, or —OC(═O)R^{D1}; wherein each instance of RD is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In some embodiments, R^{11a} and R^{11b} is each independently hydrogen, halogen, cyano, hydroxyl, substituted or unsubstituted alkyl, or —OR^{D1}; wherein each instance of R^{D1} is independently hydrogen, or substituted or unsubstituted alkyl.

In some embodiments, R^{11a} and R^{11b} are each independently hydrogen.

In one embodiment, R^{1a} and R¹ b are both hydrogen.

Groups R^{12a} and R^{12b}

In some embodiments, each of R^{2a} and R^{12b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

In some embodiments, each of R^{2a} and R^{12b} is independently hydrogen, halogen, cyano, hydroxyl, substituted or unsubstituted alkyl, substituted or unsubstituted alkenyl, —OR^{D1}, —OC(═O)R^{D1}, —NH₂, or —N(R^{D1})₂, wherein each instance of RD is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In embodiments, each of R^{2a} and R^{12b} is independently hydrogen, substituted or unsubstituted alkyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In embodiments, each of R^{2a} and R^{12b} is independently hydrogen or substituted or unsubstituted alkyl.

In certain embodiments, both R^{12a} and R^{12b} are hydrogen.

In certain embodiments, R^{12a} and R^{12b} is independently hydrogen.

Groups R^{15a} and R^{15b}

In some embodiments, each of R^{15a} and R^{15b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR_{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

In some embodiments, each of R^{15a} and R^{15b} is independently hydrogen, halogen, cyano, hydroxyl, substituted or unsubstituted alkyl, substituted or unsubstituted alkenyl, —OR^{D1}, —OC(═O)R^{D1}, —NH₂, or —N(R^{D1})₂, wherein each instance of RD is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In certain embodiments, each of R^{15a} and R^{15b} is independently hydrogen, substituted or unsubstituted alkyl, —OR^{D1}, OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In certain embodiments, each of R^{15a} and R^{15b} is independently hydrogen or substituted or unsubstituted alkyl.

In embodiments, both R^{15a} and R^{15b} are hydrogen.

In embodiments, R^{15a} and R^{15b} are each independently hydrogen.

Groups R^{16a} and R^{16b}

In some embodiments, R^{16a} and R^{6bb} is each independently hydrogen, halogen, cyano, hydroxyl, substituted or unsubstituted alkyl, substituted or unsubstituted alkenyl, substituted or unsubstituted heterocyclyl, substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

In some embodiments, R¹⁶2 and R^{16b} is each independently hydrogen, halogen, cyano, hydroxyl, substituted or unsubstituted alkyl, —OR^{D1}, or —OC(═O)R^{D1}; wherein each instance of R¹ is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

R^{16a} and R^{16b} is each independently hydrogen, halogen, cyano, hydroxyl, substituted or unsubstituted alkyl, or —OR^{D1}; wherein each instance of RD is independently hydrogen, or substituted or unsubstituted alkyl.

In one embodiment, R^{16a} and R^{16b} is each independently hydrogen, halogen, cyano, hydroxyl, substituted or unsubstituted alkyl, or —OR^{D1}; wherein each instance of R¹ is independently hydrogen, or substituted or unsubstituted alkyl.

In some embodiments, R^{16a} and R^{16b} are each independently hydrogen.

In one embodiment, R^{16a} and R^{16b} are both hydrogen.

Groups R^{37a} and R³⁷1

In some embodiments, each of R^{37a} and R^{37b} is independently hydrogen, halogen, substituted or unsubstituted alkyl, substituted or unsubstituted alkenyl, or substituted or unsubstituted alkynyl.

In some embodiments, each of R^{37a} or R^{37b} is independently hydrogen.

In some embodiments, each of R^{37a} and R^{37b} is hydrogen.

Groups R^{36a} and R^{36b}

In some embodiments, each of R^{36a} and R^{36b} is independently hydrogen, halogen, substituted or unsubstituted alkyl, substituted or unsubstituted alkenyl, or substituted or unsubstituted alkynyl.

Groups R^{35a} and R^{35b}

In some embodiments, each of R^{3a} and R^{35b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

Groups R^{34a} and R^{34b}

In some embodiments, each of R^{34a} and R^{34b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1} OC(═O)R^{D1}, —NH₂, —N(R^{D1})₂, or —NR^{D1}C(═O)R^{D1}, wherein each instance of RD is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

Groups R^{33a} and R^{33b}

In some embodiments, each of R^{33a} and R^{33b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

Groups R^{32a} and R^{32b}

In certain embodiments, each of R^{32a} and R^{32b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

Groups R^{31a} and R^{31b}

In embodiments, each R^{31a} and R^{31b} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1} —N(R^{A1})₂—N(R^{A1}), —CN(R^{A1})₂, —C(O)R^{A1}, —OC(═O)R^{A1}, or —OC(═O)OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

Groups R^{30a} and R^{30b}

In some embodiments, each of R^{30a} and R^{30b} is independently hydrogen, halogen, cyano, hydroxyl, substituted or unsubstituted alkyl, substituted or unsubstituted alkenyl, —OR^{D1}, —OC(═O)R^{D1}, —NH₂, or —N(R^{D1})₂, wherein each instance of RD is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

Groups R^{29a} and R^{29b}

In certain embodiments, R^{29a} and R^{29b} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1} N(R^{A1})₂, —N(R^{A1}), —CN(R^{A1})₂, —C(O)R^{A1}, —OC(═O)R^{A1}, or —OC(═O)OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

### Group R⁵

In some embodiments, R⁵ is hydrogen in the cis position.

In another embodiment, R⁵ is hydrogen in the trans position.

In yet another embodiment, R⁵ is methyl in the cis position.

In one embodiment, R⁵ is methyl in the trans position.

Groups R^{1a} and R^{1b}

In certain embodiments, each of R^{1a} and R^{1b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.

In certain embodiments, each of R^{1a} and R^{1b} is independently hydrogen, halogen, cyano, hydroxyl, substituted or unsubstituted alkyl, substituted or unsubstituted alkenyl, —OR^{D1}, OC(═O)R^{D1}, —NH₂, or —N(R^{D1})₂, wherein each instance of RD is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In embodiments, each of R^{1a} and R^{1b} is independently hydrogen, substituted or unsubstituted alkyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In embodiments, each of R^{1a} and R^{1b} is independently hydrogen or substituted or unsubstituted alkyl.

In some embodiments, each of R^{1a} and R^{1b} is independently hydrogen, C₁-C₆ alkyl, C₁-C₆ haloalkyl, C₁-C₆ alkoxy, C₁-C₆ alkoxyhalo, or —OH.

In some embodiments, each of R^{1a} and R^{1b} is independently —CH₃, —CH₂CH₃, —OH, —OCH₃, or —CH(CH₃)₂.

In embodiments, both R^{1a} and R^{1b} are hydrogen.

In embodiments, R^{1a} and R^{1b} are each independently hydrogen.

### Integers n, q, r, s, t, and u

In some embodiments, n is 1 or 2. In some embodiments n is 1. In another embodiment n is 2.

In some embodiments, u is 1 or 2. In some embodiments u is 1. In another embodiment u is 2.

In some embodiments, q is 0, 1, 2, or 3. In some instances q is 0, 2, or 3. In some embodiments, q is 0 or 2, in some embodiments q is 2 or 3. In some embodiments q is 1, 2, or 3. In some embodiments q is 0, 1, or 2. In some embodiments q is 0. In some embodiments q is 1. In some embodiments, q is 2. In some embodiments, q is 3.

In some embodiments, r is 0, 1 or, 2. In some instances r is 0, or 2. In some embodiments r is 1, or 2. In some embodiments r is 0, or 1. In some embodiments r is 0. In some embodiments r is 1. In some embodiments, r is 2.

In some embodiments, s is 0, 1 or, 2. In some instances s is 0, or 2. In some embodiments s is 1, or 2. In some embodiments s is 0, or 1. In some embodiments s is 0. In some embodiments s is 1. In some embodiments, s is 2.

In some embodiments, t is 0, 1, 2, or 3. In some instances t is 0, 2, or 3. In some embodiments, t is 0 or 2, in some embodiments t is 2 or 3. In some embodiments t is 1, 2, or 3. In some embodiments t is 0, 1, or 2. In some embodiments t is 0. In some embodiments t is 1. In some embodiments, t is 2. In some embodiments, t is 3.

In some embodiments, r is 1 and s is 1.

In some embodiments, q is 0, 2, or 3; and t is 0, 2, or 3. In another embodiment q is 2, t is 2, and u is 1.

In some embodiments, q is 0, 2, or 3; and u is 1. In another embodiment q is 0, 2, or 3; t is 0, 2, or 3, and u is 1.

In another embodiment q is 1, t is 0, 2, or 3, and u is 2.

In some embodiments q, u, r, s, and t are not simultaneously 1. In some embodiments, when t is 0, 2, or 3 then q, u, s, and r are not simultaneously 1. In some embodiments, when q is 0 or 2 and u is 1, then t, s, and r are not simultaneously 1. In some embodiments, when u is 2 and q is 1, then t, s, and r are not simultaneously 1. In some embodiments, when r is 0 or 2, then q, u, s, and t are not simultaneously 1. In some embodiments, when s is 0 or 2, then q, u, r, and t are not simultaneously 1.

### Group R³

In some embodiments, R³ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In some embodiments, R³ is substituted or unsubstituted alkyl. In some embodiments, alkyl is optionally substituted with halo, or OR^{D1}

In certain embodiments, R³ is substituted or unsubstituted alkyl.

In some embodiments, R³ is substituted alkyl.

In some embodiments, R³ is unsubstituted alkyl.

In some embodiments, R³ is methyl.

In some embodiments, R³ is —OCH₃.

In some embodiments, R³ is —CH₂OCH₃ or —CH₂OCH₂CH₃.

### Group R^{3a}

In some embodiments, R^{3a} is hydrogen, substituted or unsubstituted alkyl, substituted or unsubstituted carbocyclyl, substituted or unsubstituted heterocyclyl, substituted or unsubstituted aryl, or substituted or unsubstituted heteroaryl.

In some embodiments, R^{3a} is substituted or unsubstituted alkyl. In some embodiments, alkyl is optionally substituted with halo, or OR^{D1}.

In certain embodiments, R^{3a} is substituted or unsubstituted alkyl.

In some embodiments, R^{3a} is substituted alkyl.

In some embodiments, R^{3a} is unsubstituted alkyl.

In some embodiments, R^{3a} is methyl.

In some embodiments, R^{3a} is —OCH₃.

In some embodiments, R^{3a} is —CH₂OCH₃ or —CH₂OCH₂CH₃.

### Group R^{D1}

In one embodiment, R^{D1} is hydrogen or substituted or unsubstituted alkyl.

### Group R¹⁹

In some embodiments, R¹⁹ is substituted alkyl.

In some embodiments, R¹⁹ is unsubstituted alkyl.

In another embodiment, R¹⁹ is methyl or hydrogen. In yet another embodiment, R¹⁹ is methyl, ethyl or hydrogen.

In some embodiments, R¹⁹ is hydrogen.

In some embodiments, R¹⁹ is methyl.

In some embodiments, R¹⁹ is substituted alkyl.

In some embodiments, R⁹ is unsubstituted alkyl.

In certain embodiments, R¹⁹ is methyl.

In embodiments, R¹⁹ is —CH₂OCH₃.

In embodiments, R¹⁹ is —OCH₃.

In certain embodiments, R¹⁹ is ethyl.

In embodiments, R¹⁹ is hydrogen.

### Group X

In some embodiments, X is hydrogen, substituted or unsubstituted alkyl, substituted or unsubstituted alkenyl, substituted or unsubstituted aryl, substituted or unsubstituted heterocyclyl, or substituted or unsubstituted heteroaryl.

In some embodiments, X is hydrogen, substituted or unsubstituted heteroaryl, or substituted or unsubstituted alkyl.

In another embodiment, X is a substituted or unsubstituted heteroaryl.

In one embodiment, X is a substituted or unsubstituted 5-10 membered heteroaryl.

In one embodiment, X is hydrogen, halogen, substituted or unsubstituted alkyl, substituted or unsubstituted heterocyclyl, substituted or unsubstituted carbocyclyl, substituted or unsubstituted aryl, substituted or unsubstituted heteroaryl, or —OR^{A1}; wherein R^{A1} is independently hydrogen, substituted or unsubstituted alkyl, substituted or unsubstituted carbocyclyl, substituted or unsubstituted aryl, or substituted or unsubstituted heteroaryl.

In some embodiments, X is hydrogen, or substituted or unsubstituted heteroaryl.

For example, in some instances X is a substituted or unsubstituted N-linked heteroaryl.

In one embodiment, N-linked heteroaryl is a 5-6 membered N-linked heteroaryl.

In some embodiments, X is:

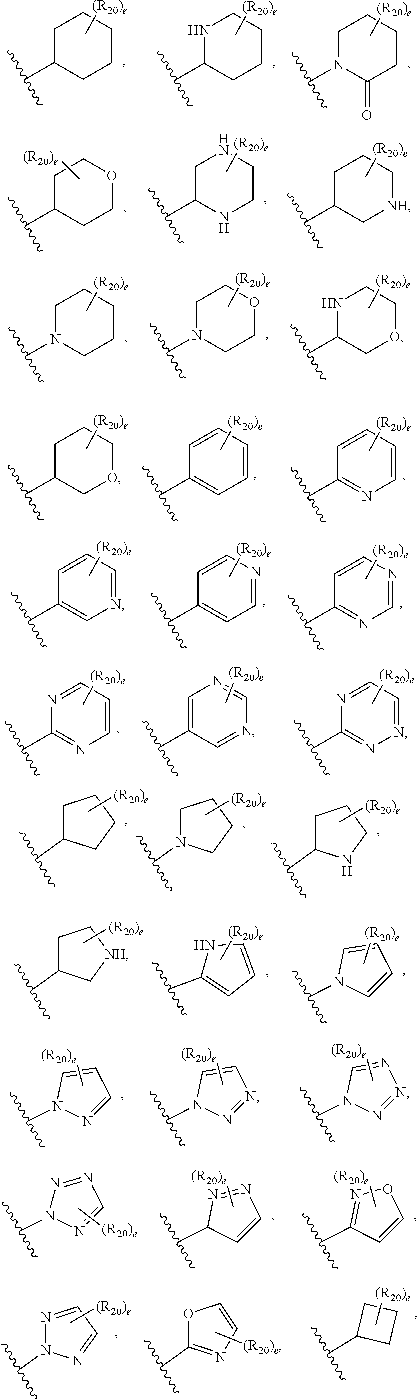

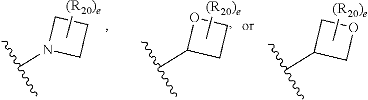

wherein each instance of R₂₀ is independently halogen, —NO₂, —CN, —OR^{GA}, —N(R^{GA})₂, —C(═O)R^{GA}, —C(═O)OR^{GA}, —OC(═O)R^{GA}, —OC(═O)OR^{GA}, —C(═O)N(R^{GA})₂, —N(R^{GA})C(═O)R^{GA}, —OC(═O)N(R^{GA})₂, —N(R^{GA})C(═O)OR^{GA}, —S(═O)₂R^{GA}, —S(═O)₂OR^{GA}, —OS(═O)₂R^{GA}, —S(═O)₂N(R^{GA})₂, or —N(R^{GA})S(═O)₂R^{GA}; substituted or unsubstituted C₁₋₆ alkyl, substituted or unsubstituted C₂₋₆ alkenyl, substituted or unsubstituted C₂₋₆ alkynyl, substituted or unsubstituted C₃₋₄ carbocylyl, or substituted or unsubstituted 3- to 4-membered heterocylyl;
wherein each instance of R^{GA} is independently hydrogen, substituted or unsubstituted C₁₋₆ alkyl, substituted or unsubstituted C₂₋₈ alkenyl, substituted or unsubstituted C₂₋₆ alkynyl, substituted or unsubstituted C₃₋₆ carbocylyl, substituted or unsubstituted 3-6 membered heterocylyl, substituted or unsubstituted aryl, substituted or unsubstituted heteroaryl, an oxygen protecting group when attached to oxygen, or a nitrogen protecting group when attached to nitrogen; or two R^{GA} groups are taken with the intervening atoms to form a substituted or unsubstituted carbocyclic or substituted or unsubstituted heterocyclic ring; and
e is 0, 1, 2, 3, 4, or 5.

In some embodiments, X is:

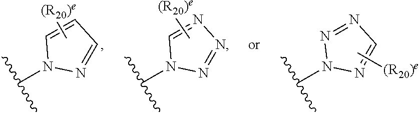

wherein each instance of R²⁰ is, independently, halogen, —NO₂, —CN, —OR^{GA}, —N(R^{GA})₂, —C(═O)R^{GA}, —C(═O)OR^{GA}, —C(═O)N(R^{GA})₂, —N(R^{GA})C(═O)R^{GA}, —OC(═O)N(R^{GA})₂, substituted or unsubstituted C₁₋₆ alkyl, substituted or unsubstituted 3-4 membered carbocylyl, substituted or unsubstituted 3-4 membered heterocyclyl;
wherein each instance of R^{GA} is independently hydrogen, substituted or unsubstituted C₁₋₆ alkyl, substituted or unsubstituted aryl, substituted or unsubstituted heteroaryl, an oxygen protecting group when attached to oxygen, a nitrogen protecting group when attached to nitrogen, or two R^{GA} groups are taken with the intervening atoms to form a substituted or unsubstituted carbocyclic or heterocyclic ring; and
e is 0, 1, 2, or 3.

In some embodiments X is:

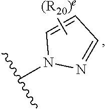

wherein each instance of R₂₀ is, independently, halogen, —NO₂, —CN, —OR^{GA}, —N(R^{GA})₂, —C(═O)R^{GA}, —C(═O)OR^{GA}, —C(═O)N(R^{GA})₂, —N(R^{GA})C(═O)R^{GA}, —OC(═O)N(R^{GA})₂, substituted or unsubstituted C₁₋₆ alkyl, substituted or unsubstituted 3-4 membered carbocylyl, substituted or unsubstituted 3-4 membered heterocyclyl;
wherein each instance of R^{GA} is independently hydrogen, substituted or unsubstituted C₁₋₆ alkyl, substituted or unsubstituted aryl, substituted or unsubstituted heteroaryl, or two R^{GA} groups are taken with the intervening atoms to form a substituted or unsubstituted carbocyclic or heterocyclic ring; and
e is 0, 1, 2, or 3.

### Group R²⁸

In some embodiments, R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl.

In some embodiments, R²⁸ is hydrogen, substituted or unsubstituted alkyl, substituted or unsubstituted heteroaryl.

In some embodiments, R²⁸ is hydrogen.

In some embodiments, R²⁸ is methyl.

In some embodiments, R²⁸ is selected from the group consisting of:

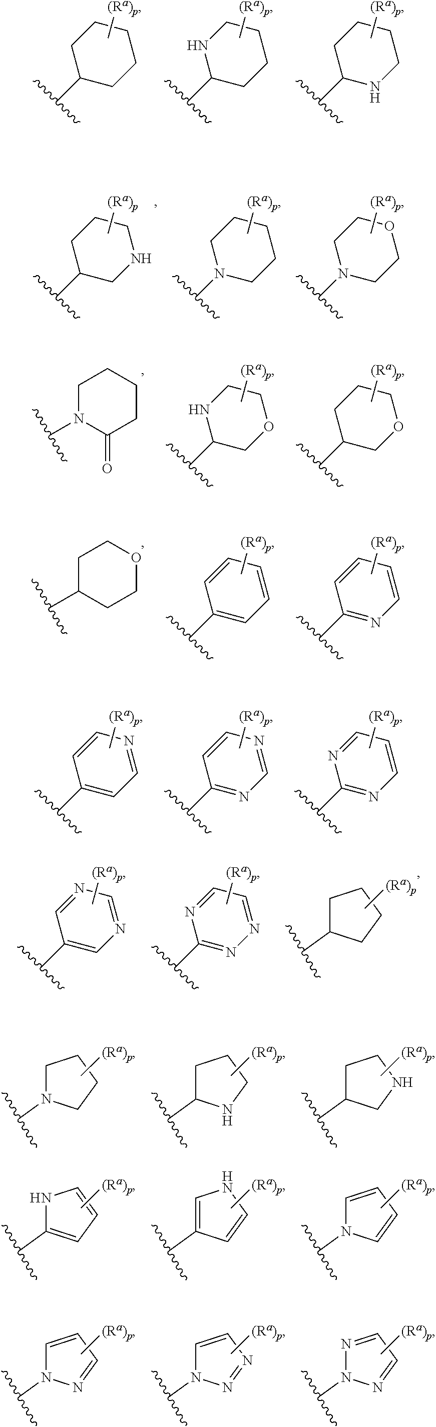

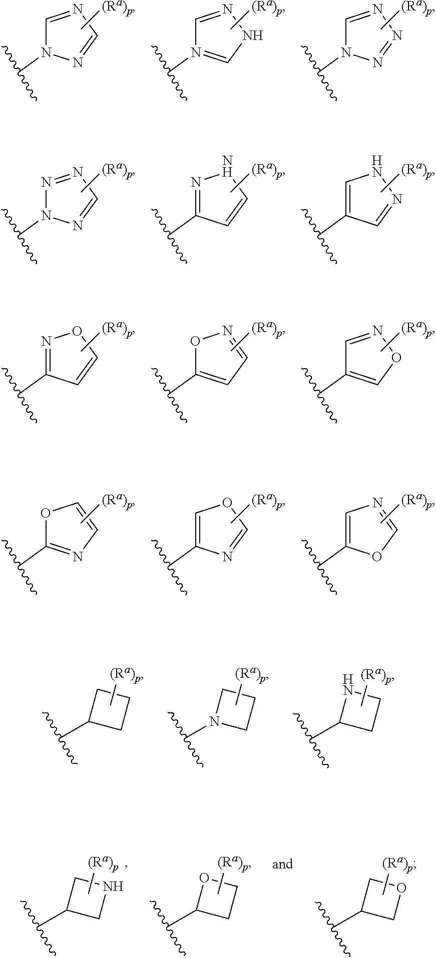

wherein:
each instance of R^{a} is independently hydrogen, halogen, —NO₂, —CN, —OR^{D4}, —N(R^{D4})₂, —C(═O)R^{D4}, —C(═O)OR^{D4}, —C(═O)N(R^{D4})₂, —OC(═O)R^{D4}, —OC(═O)OR^{D4}, —N(R^{D4})C(═O)R^{D4}, —OC(═O)N(R^{D4})₂, —N(R^{D4})C(═O)OR^{D4}, —S(═O)₂R^{D4}, —S(═O)₂OR^{D4}, —OS(═O)₂R^{D4}, —S(═O)₂N(R^{D4})₂, or —N(R^{D4})S(═O)₂R^{D4}, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl;
each instance of R^{D4} is independently hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl, an oxygen protecting group when attached to oxygen, a nitrogen protecting group when attached to nitrogen, or two R^{D4} groups are taken with the intervening atoms to form a substituted or unsubstituted heterocyclic ring; and
p is an integer selected from 0 to 11.

In some embodiments, R²⁸ is selected from the group consisting of.

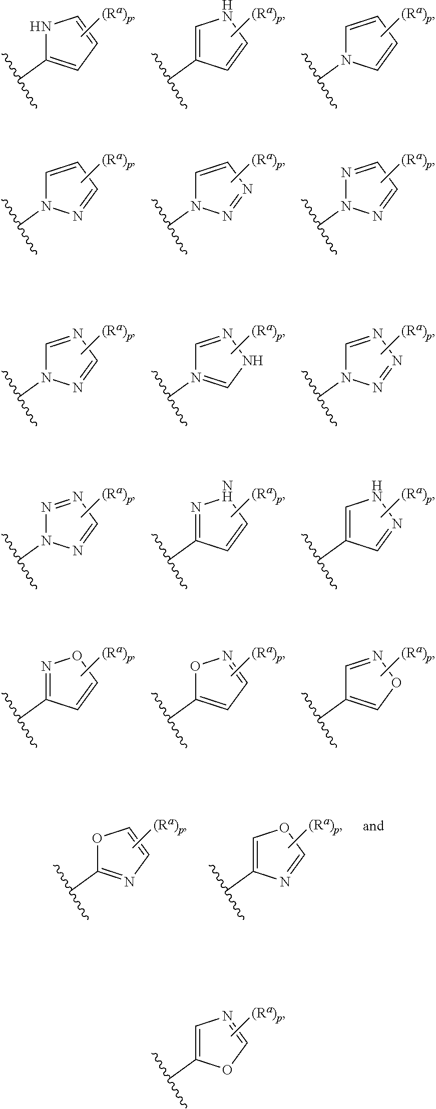

wherein R^{a} and p is as defined herein.

In certain embodiments, R²⁸ is

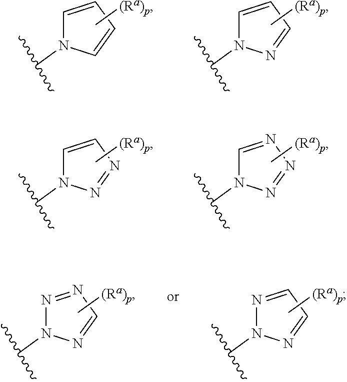

wherein R^{a} and p is as defined herein.

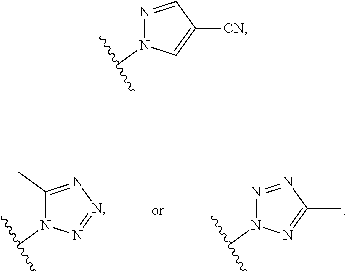

In certain embodiments, R²⁸ is

### Group R₂₀

In some embodiments, R₂₀ is —CN.

In some embodiments, R²⁰ is unsubstituted alkyl.

In another embodiment, R²⁰ is unsubstituted C₁₋₆alkyl.

In one embodiment, R₂₀ is methyl.

### Group R⁵⁵

In some embodiments, R⁵⁵ is hydrogen, halogen, cyano, or substituted or unsubstituted alkyl.

In some embodiments R⁵⁵ is cyano. In another embodiment, R⁵⁵ is methyl. In one example R⁵⁵ is hydrogen. In another example R⁵⁵ is halogen.

### Group R¹⁸

In some embodiments, R¹⁸ is unsubstituted alkyl.

In another embodiment, R¹⁸ is substituted alkyl.

In one embodiment, R¹⁸ is substituted or unsubstituted C₁₋₄ alkyl.

In some embodiments, R¹⁸ is unsubstituted C₁₋₄ alkyl.

In some embodiments, R¹⁸ is methyl.

In some embodiments, the compound of Formula (1-I) is a compound of is a compound of Formula (1-II-a), Formula (1-II-b), Formula (1-II-c), or Formula (1-II-d):

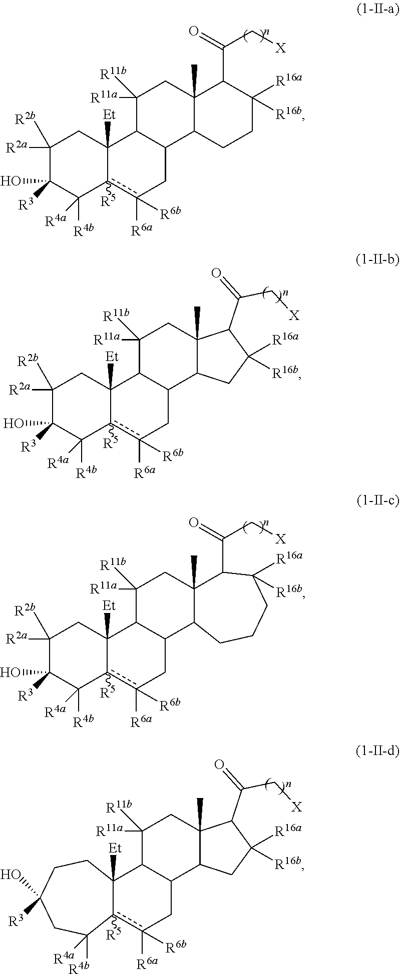

or a pharmaceutically acceptable salt thereof.

In some embodiments, the compound of Formula (1-I) is a compound of Formula (1-III-a), Formula (1-III-b), Formula (1-III-c), or Formula (1-IIId):

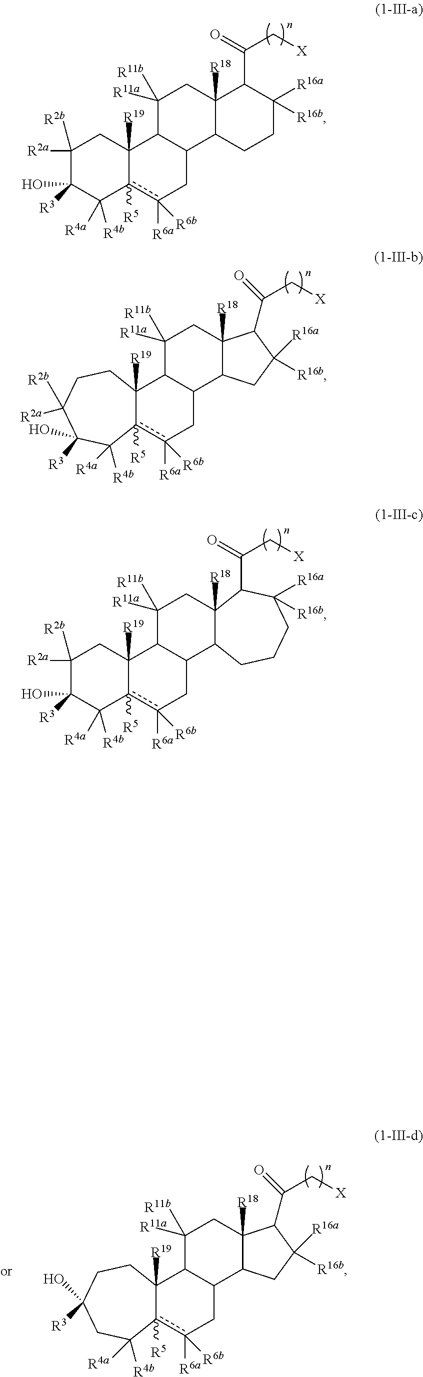

or a pharmaceutically acceptable salt thereof.

In some embodiments, the compound of Formula (1-I) is a compound of Formula (1-IV-a), Formula (1-IV-b), Formula (1-IV-c), or Formula (1-IV-d):

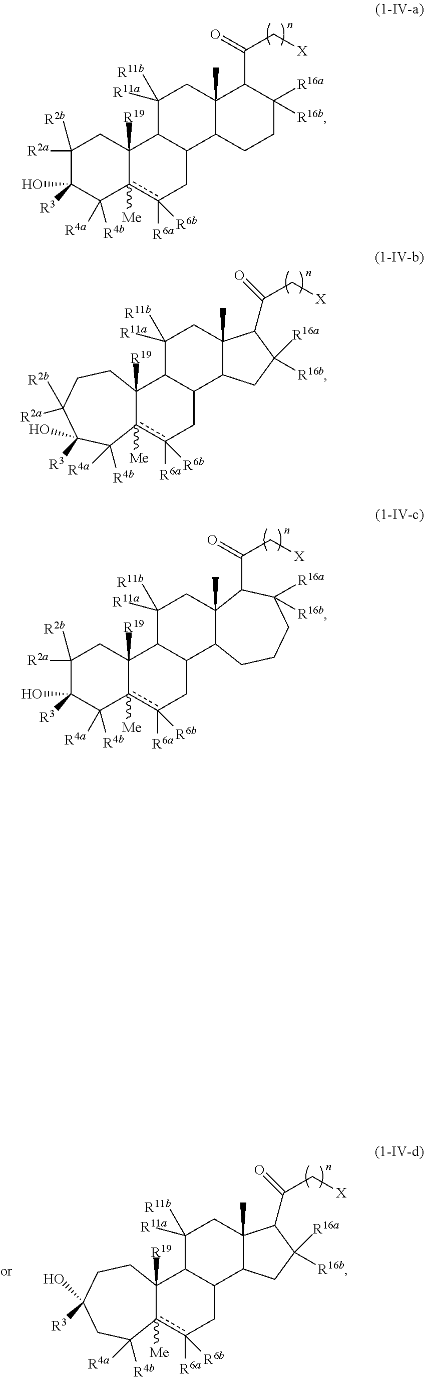

or a pharmaceutically acceptable salt thereof.

In some embodiments, the compound of Formula (1-I) is a compound of Formula

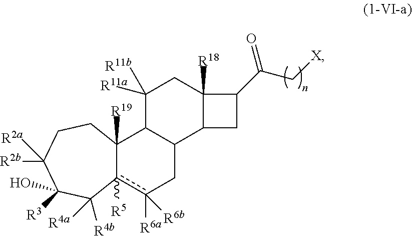

or a pharmaceutically acceptable salt thereof.

In some embodiments, the compound of Formula (1-I) is a compound of Formula

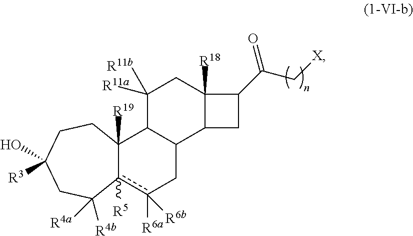

or a pharmaceutically acceptable salt thereof.

In some embodiments, the compound of Formula (1-I) is a compound of Formula (1-VII-a):

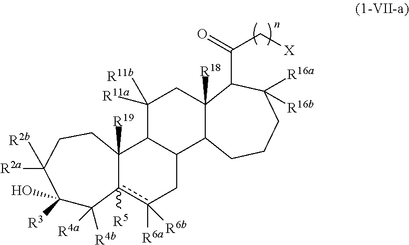

or a pharmaceutically acceptable salt thereof.

In some embodiments, the compound of Formula (1-I) is a compound of Formula (1-VII-b):

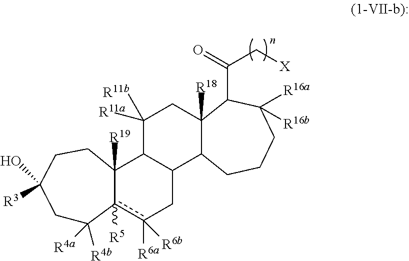

or a pharmaceutically acceptable salt thereof.

In some embodiments, the compound of Formula (1-T) is a compound of Formula (1-VIII-a) or Formula (1-VIII-b):

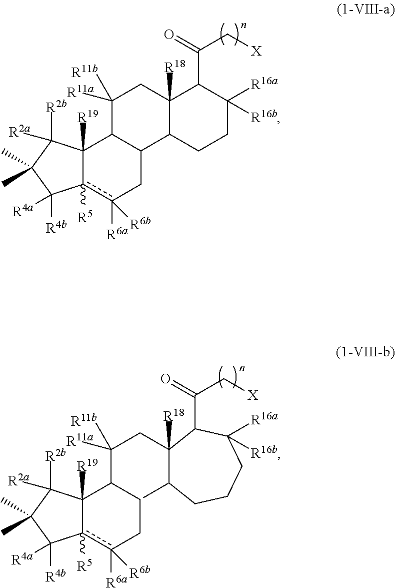

or a pharmaceutically acceptable salt thereof.

In some embodiments, the compound of Formula (1-I) is a compound of Formula (1-IX-a), Formula (1-IX-b), Formula (1-IX-c), or Formula (1-IX-d):

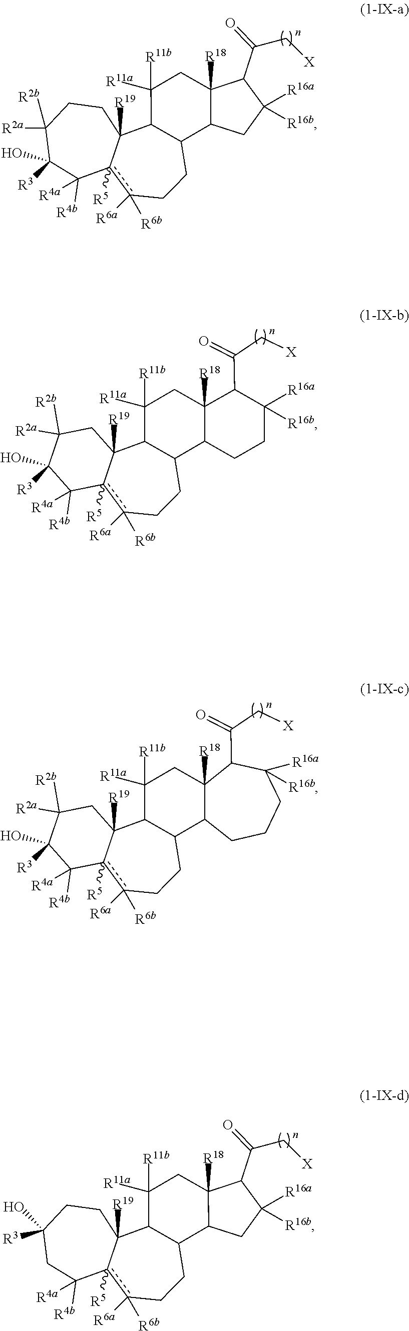

or a pharmaceutically acceptable salt thereof.

In some embodiments, the compound of Formula (1-I) is a compound of Formula (1-X-a), Formula (1-X-b), Formula (1-X-c), or Formula (1-X-d):

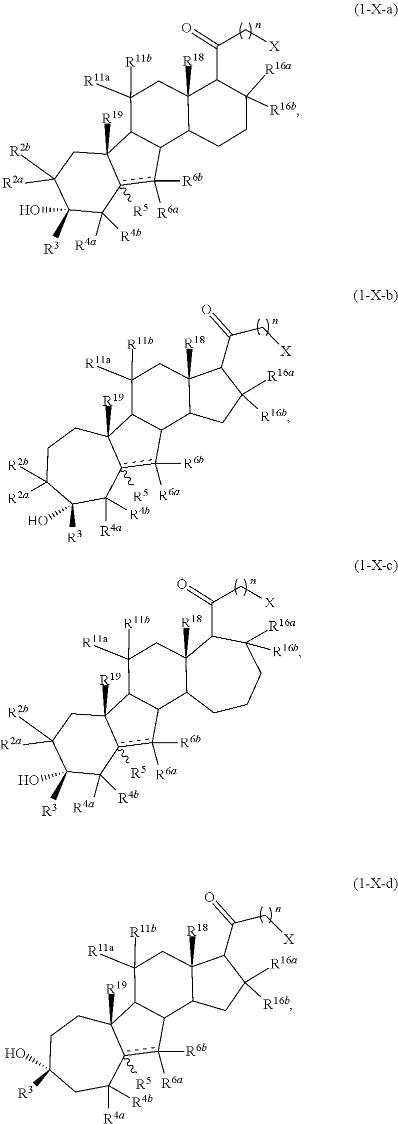

or a pharmaceutically acceptable salt thereof.

In some embodiments, the compound of Formula (1-1) is a compound of Formula (1-XI-a), Formula (1-XI-b), Formula (1-XI-c), or Formula (I-XI-d):

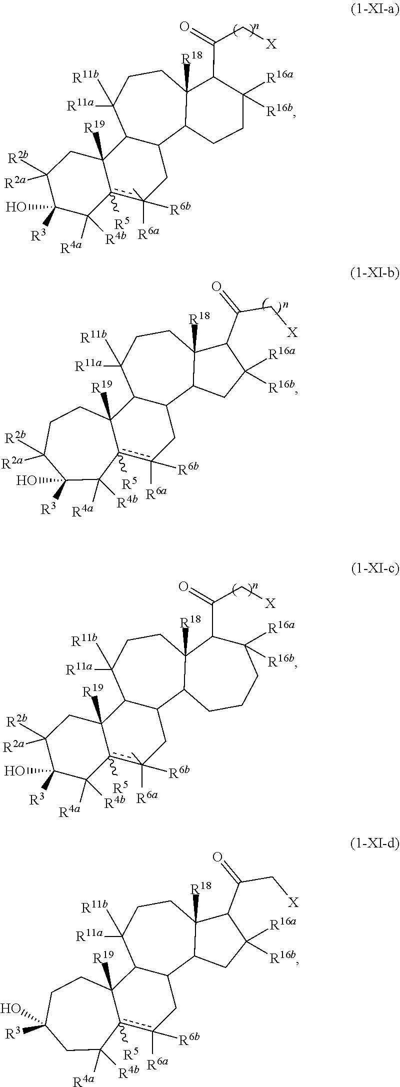

or a pharmaceutically acceptable salt thereof.

In some embodiments, the compound of Formula (1-I) is a compound of Formula —XII-a), -or Formula (1-XII-b):

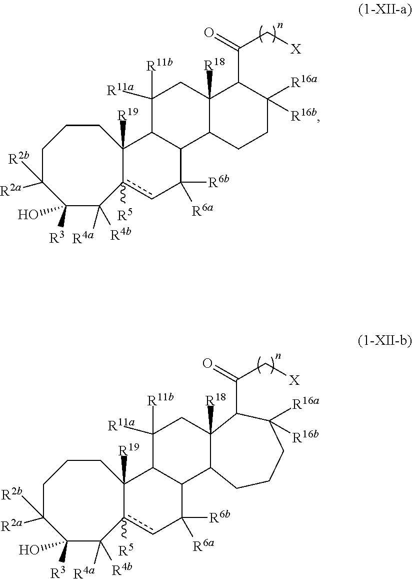

or a pharmaceutically acceptable salt thereof.

In some embodiments, the compound of Formula (1-I) is a compound of Formula (1-XIII-a) or Formula (1-XIII-b):

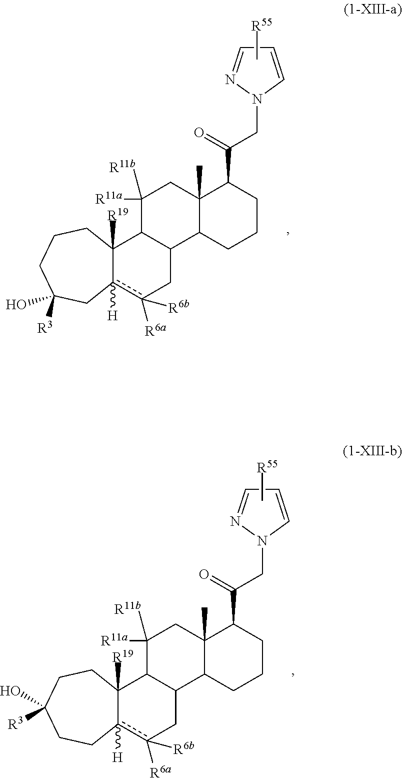

or a pharmaceutically acceptable salt thereof; wherein R⁵⁵ is hydrogen, halogen, cyano, or substituted or unsubstituted alkyl.

In some embodiments, the compound of Formula (1-I) is a compound of Formula (1-XIV-a):

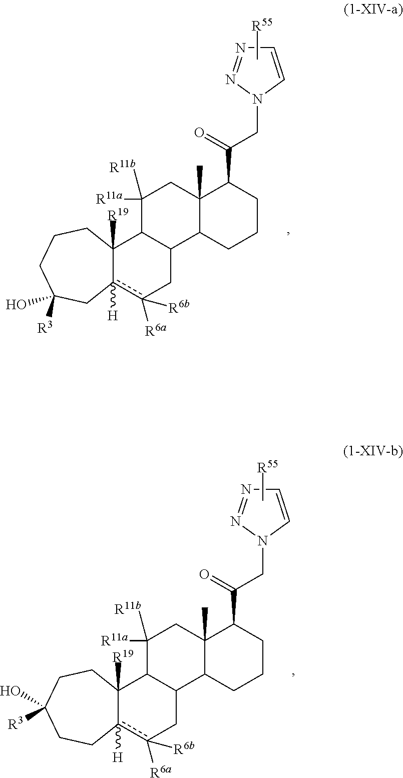

or a pharmaceutically acceptable salt thereof;
wherein R⁵⁵ is hydrogen, halogen, cyano, or substituted or unsubstituted alkyl.

In some embodiments, the compound of Formula 2-I is the compound of Formula 2-Ic:

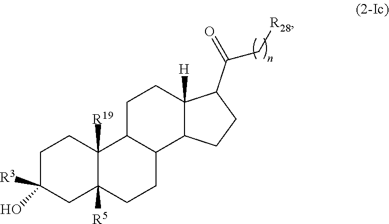

or a pharmaceutically acceptable salt thereof.

In some embodiments, the compound is of Formula 2-Id:

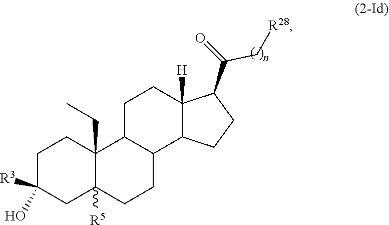

or a pharmaceutically acceptable salt thereof.

In an embodiment, the compound of Formula 2-I is the compound of Formula 2-Ie

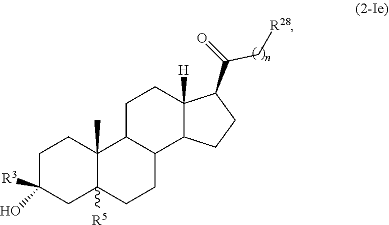

or a pharmaceutically acceptable salt thereof.

In an embodiment, the compound of Formula 2-I is the compound of Formula 2-If:

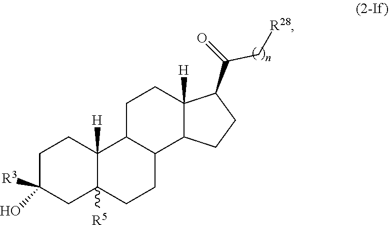

or a pharmaceutically acceptable salt thereof.

In an embodiment, the compound of Formula 2-I is the compound of Formula 2-Ig, Formula 2-1g-11, or Formula 2-1h:

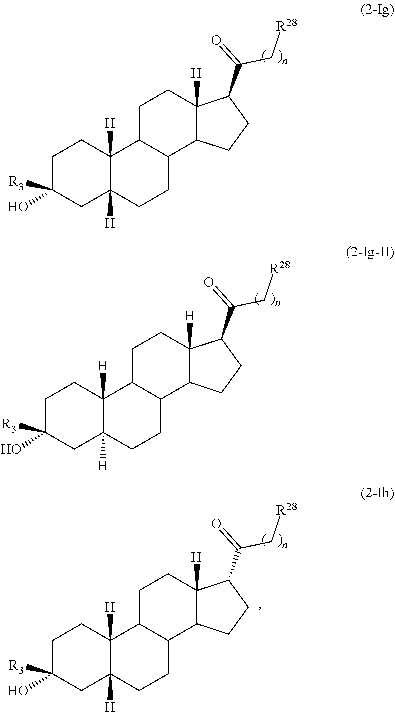

or a pharmaceutically acceptable salt thereof.

In some embodiments, the compound of Formula 2-1 is the compound of Formula 2-Iga or Formula 2-Iha:

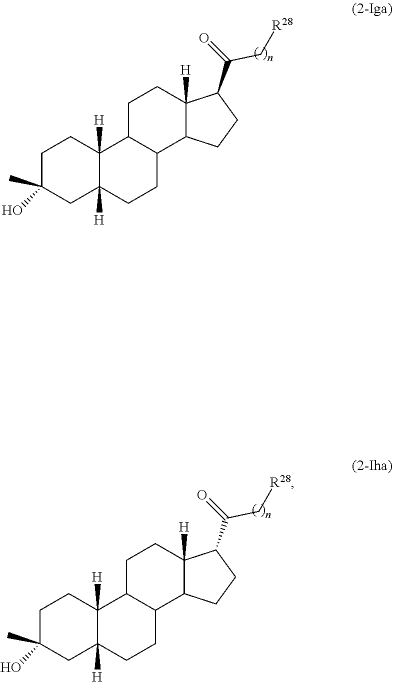

or a pharmaceutically acceptable salt thereof.

In some embodiments, the compound of Formula 2-I is the compound of Formula 2-Igb or Formula 2-Ihb:

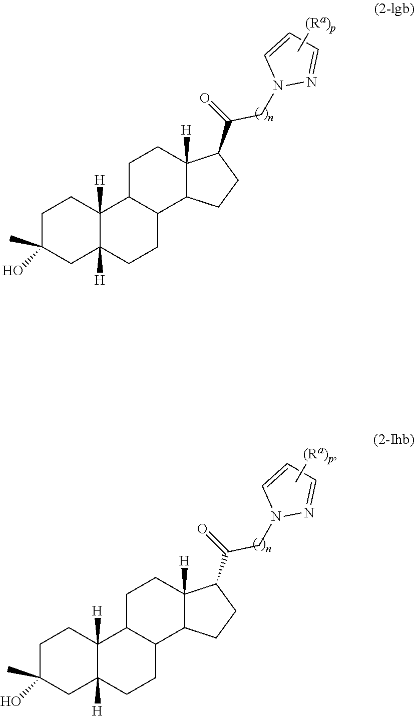

or a pharmaceutically acceptable salt thereof.

In some embodiments, the compound of Formula 2-I is the compound of Formula 2-Igc:

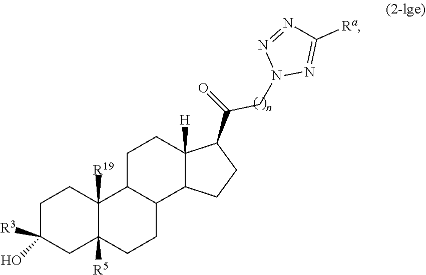

or a pharmaceutically acceptable salt thereof.

In some embodiments, the compound of Formula 2-I is the compound of Formula 2-Igd:

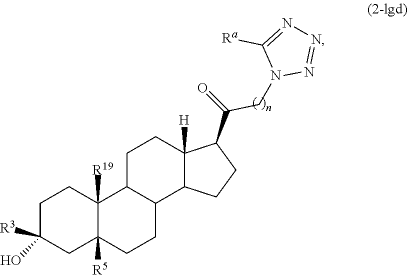

or a pharmaceutically acceptable salt thereof.

It should be appreciated that the stereochemistry at C17 could be depicted in any of the following but equivalent ways:

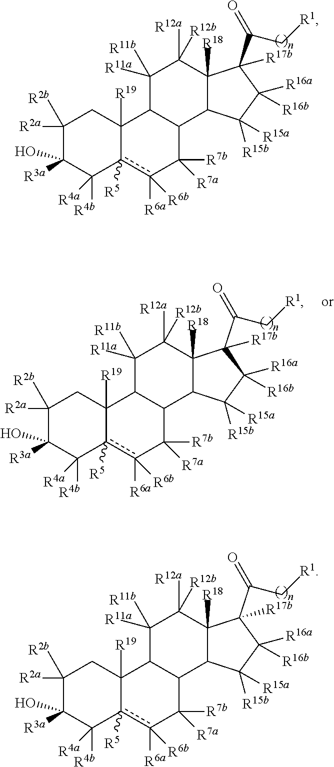

In some embodiments, the compound of Formula 3-Ill is the compound is of Formula 3-III-bd:

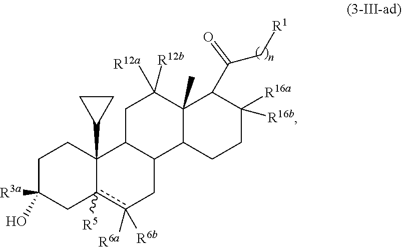

or a pharmaceutically acceptable salt thereof.

In some embodiments, the compound of Formula 3-III is the compound is of Formula 3-III-bd:

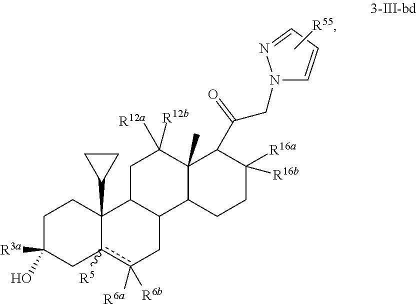

or a pharmaceutically acceptable salt thereof. In some embodiments, R⁵⁵ is hydrogen, halogen, cyano, or substituted or unsubstituted alkyl. In some embodiments, R⁵⁵ is hydrogen. In some embodiments, R⁵⁵ is halogen. In some embodiments, R⁵⁵ is cyano. In some embodiments, R⁵⁵ is unsubstituted alkyl. In some embodiments, R⁵⁵ is substituted alkyl.

In some embodiments, a pharmaceutical composition comprises a compound described herein or pharmaceutically acceptable salt thereof, and a pharmaceutically acceptable excipient.

In some embodiments, a method of treating a CNS-related disorder in a subject in need thereof, comprises administering to the subject an effective amount of a compound described herein or a pharmaceutically acceptable salt thereof. In some embodiments, the CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, or status epilepticus. In some embodiments, the CNS-related disorder is depression. In some embodiments, the CNS-related disorder is postpartum depression. In some embodiments, the CNS-related disorder is major depressive disorder. In some embodiments, the major depressive disorder is moderate major depressive disorder. In some embodiments, the major depressive disorder is severe major depressive disorder.

In some embodiments, the compound is selected from the group consisting of the compounds identified in Table 1 below:

TABLE 1

| Example | STRUCTURE/ID |
|---|---|
| 1 | 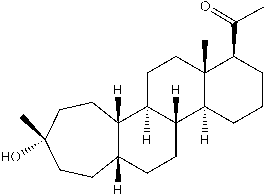 |
| 3 | 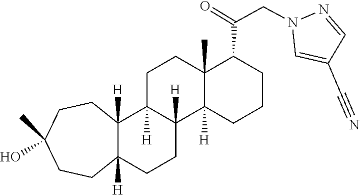 |
| 4 | 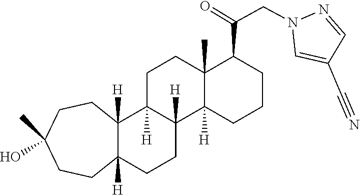 |
| 9 | 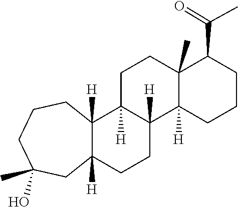 |
| 10 | 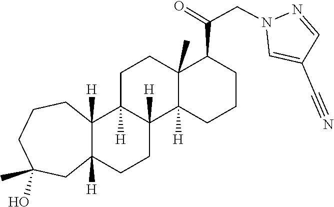 |
| 11 | 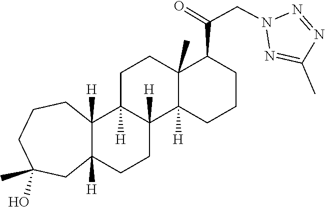 |
| 12 | 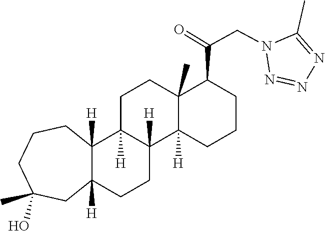 |
| 13 | 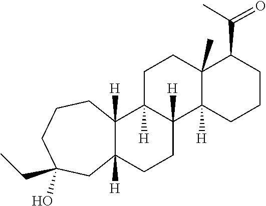 |
| 14 | 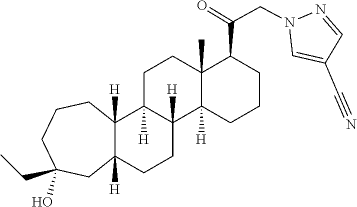 |
| 15 | 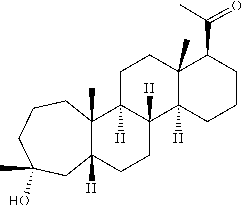 |
| 16 | 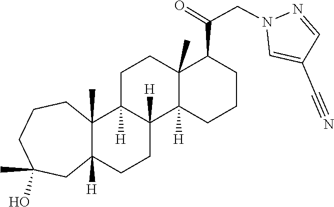 |
| 17 | 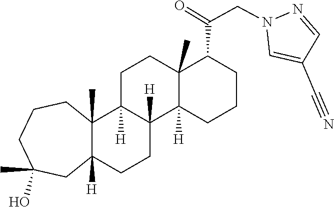 |
| 18 | 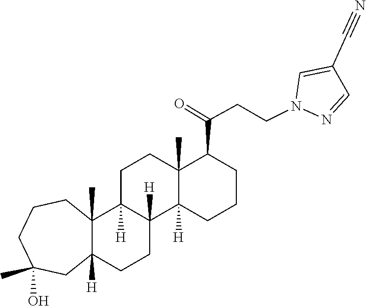 |
| 19 | 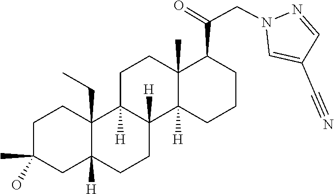 |
| 20 | 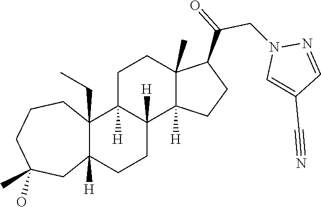 |
| 21 | 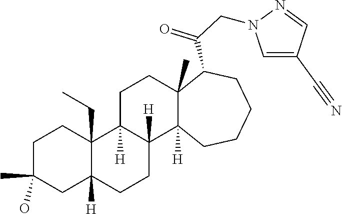 |
| 22 | 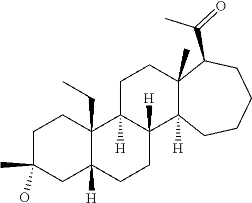 |
| 23 | 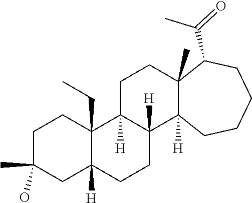 |
| 24 | 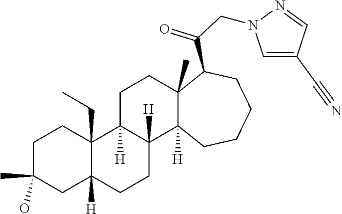 |
| 25 | 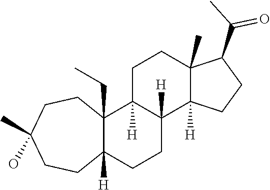 |
| 26 | 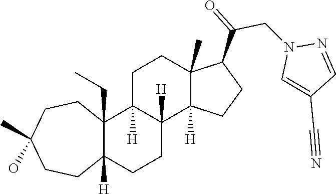 |
| 27 | 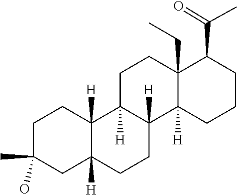 |
| 28 | 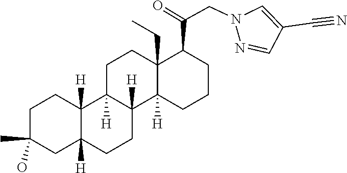 |
| 29 | 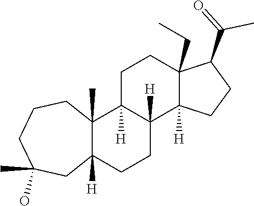 |
| 30 | 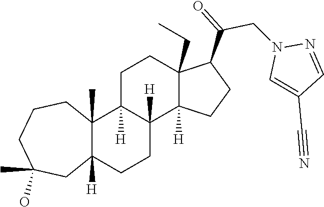 |
| 31 | 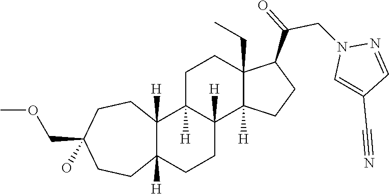 |
| 32 | 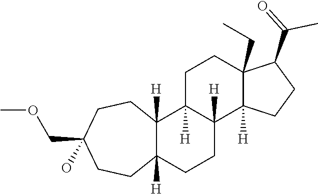 |
| 33 | 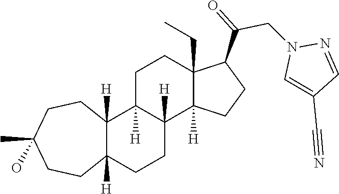 |
| 34 | 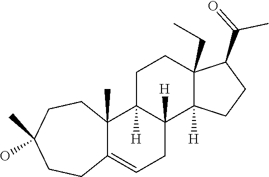 |
| 35 | 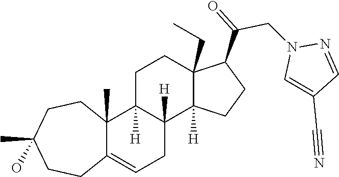 |
| 36 | 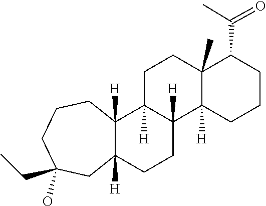 |
| 37 | 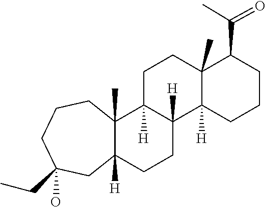 |
| 38 | 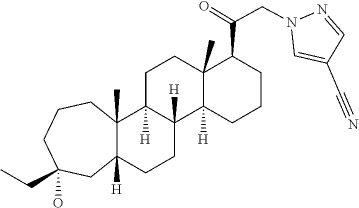 |
| 39 | 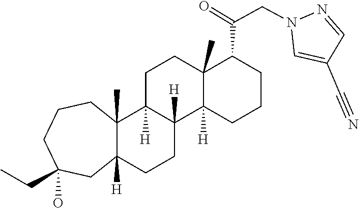 |
| 40 | 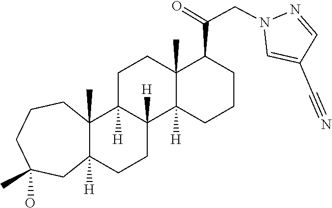 |
| 41 | 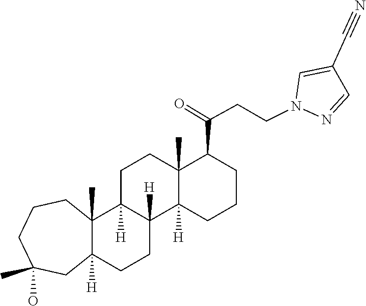 |
| 42 | 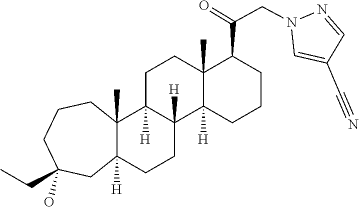 |
| 43 | 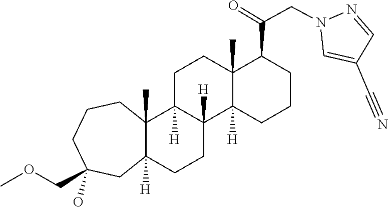 |
| 44 | 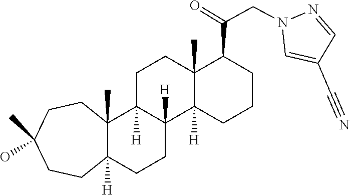 |
| 45 | 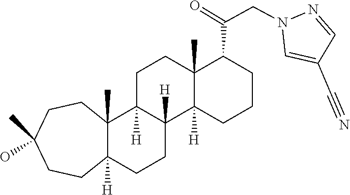 |
| 46 | 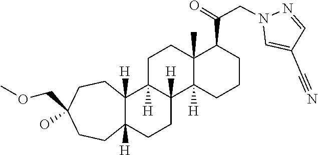 |
| 47 | 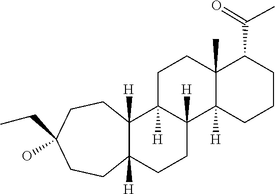 |
| 48 | 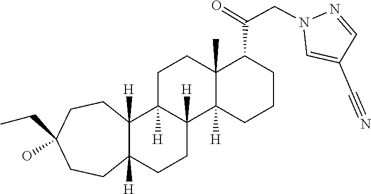 |
| 49 | 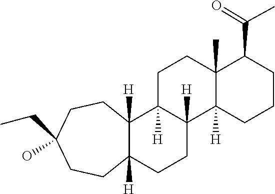 |
| 50 | 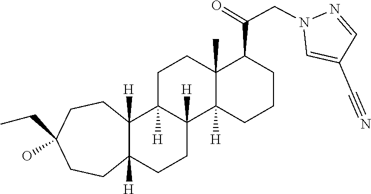 |
| 51 | 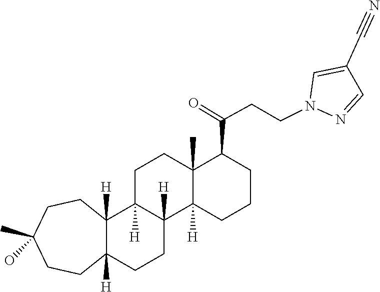 |
| 52 | 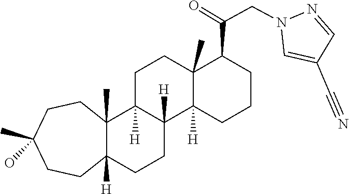 |
| 53 | 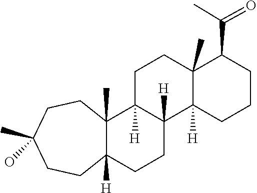 |
| 54 | 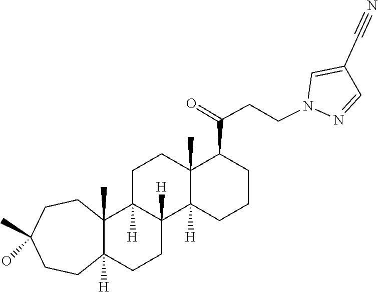 |
| 55 | 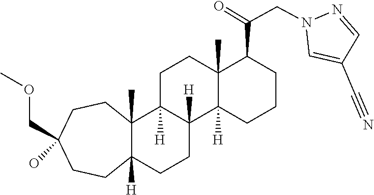 |
| 56 | 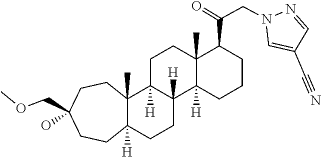 |
| 57 | 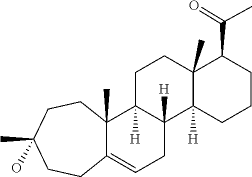 |
| 58 | 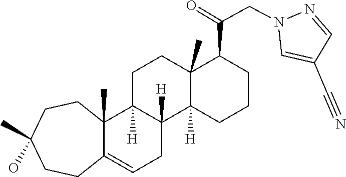 |
| 59 | 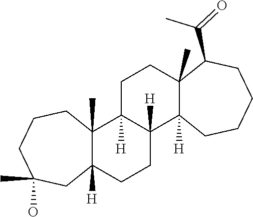 |
| 60 | 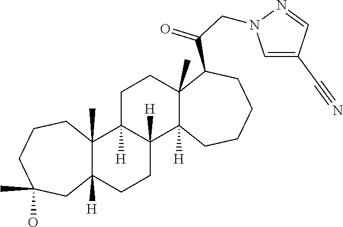 |
| 61 | 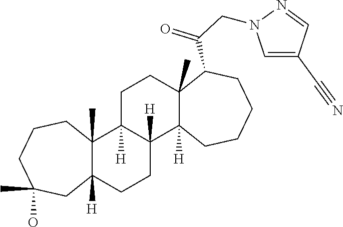 |
| 62 | 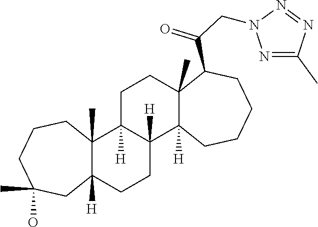 |
| 63 | 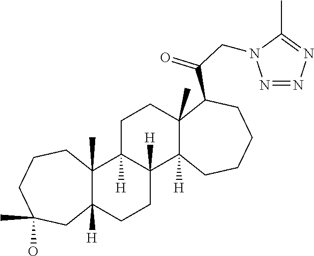 |
| 64 | 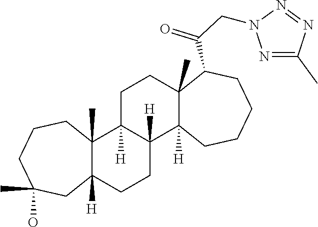 |
| 65 | 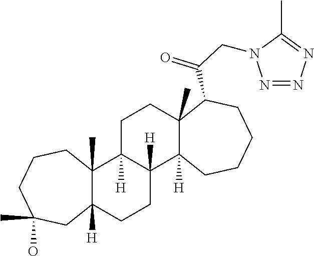 |

In some embodiments, the compound is selected from the group consisting of the compounds identified in Table 2 below:

TABLE 2

| Example | Structure |
|---|---|
| 66 | 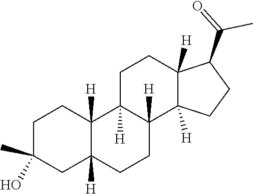 |
| 67 | 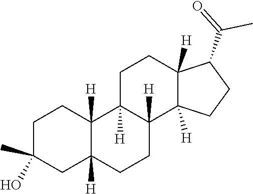 |
| 68 | 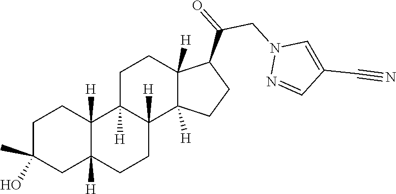 |
| 69 | 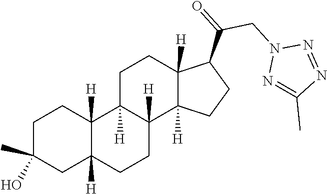 |
| 70 | 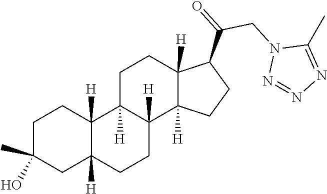 |
| 71 | 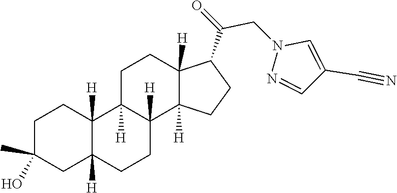 |
| 72 | 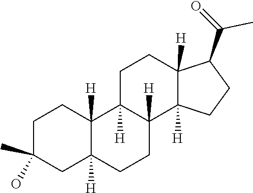 |
| 73 | 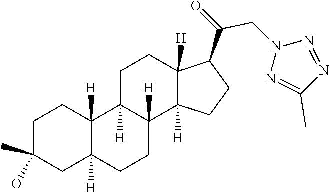 |
| 74 | 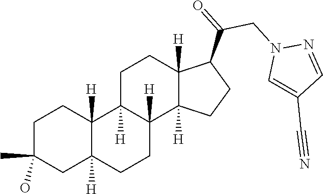 |
| 75 | 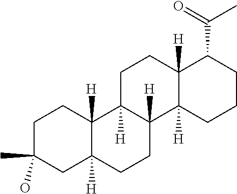 |
| 76 | 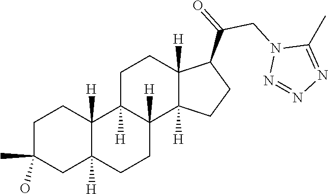 |
| 77 | 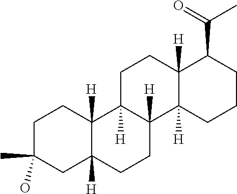 |
| 78 | 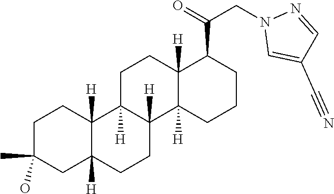 |
| 79 | 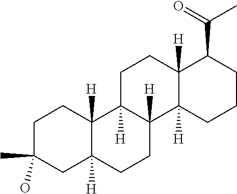 |
| 80 | 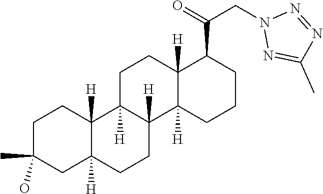 |
| 81 | 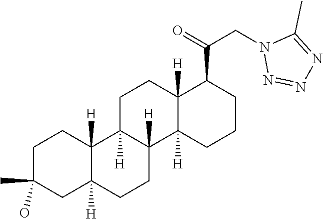 |
| 82 | 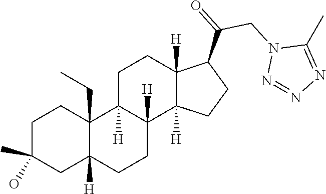 |
| 83 | 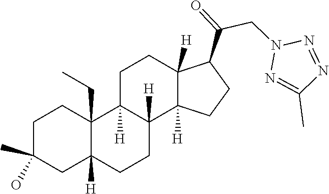 |
| 84 | 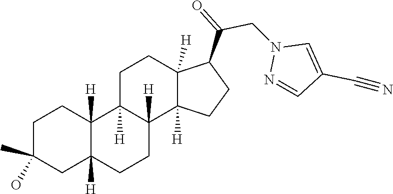 |

In some embodiments, the compound is selected from the group consisting of the compounds identified in Table 3 below:

TABLE 3

| Example | Structure |
|---|---|
| 85 | 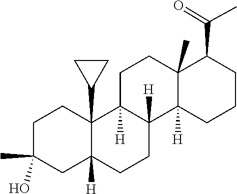 |
| 86 | 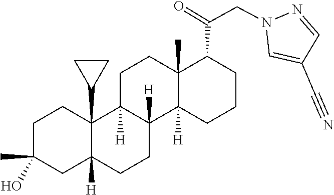 |
| 87 | 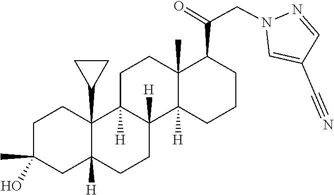 |
| 88 | 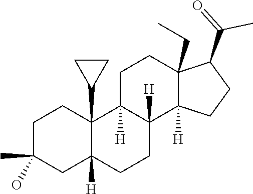 |
| 89 | 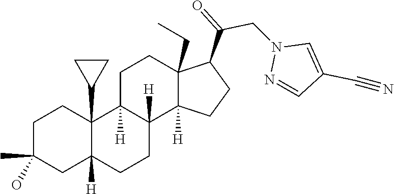 |
| 90 | 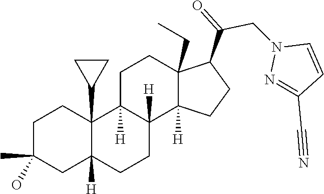 |
| 91 | 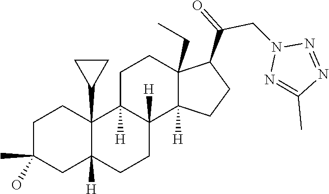 |
| 92 | 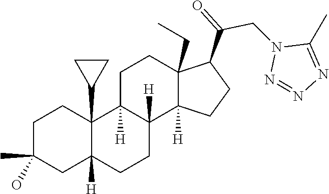 |

In one aspect, provided herein is a pharmaceutically acceptable salt of a compound described herein (e.g., a compound of Formulae (1-I), (2-I) or (3-I)).

In one aspect, provided herein is a pharmaceutical composition comprising a compound described herein (e.g., a compound of Formulae (1-I), (2-I) or (3-I)) or a pharmaceutically acceptable salt thereof, and a pharmaceutically acceptable excipient. In certain embodiments, the compound of the present invention is provided in an effective amount in the pharmaceutical composition. In certain embodiments, the compound of the present invention is provided in a therapeutically effective amount.

Compounds of the present invention as described herein, act, in certain embodiments, as GAB_{A} modulators, e.g., effecting the GABAA receptor in either a positive or negative manner. As modulators of the excitability of the central nervous system (CNS), as mediated by their ability to modulate GABAA receptor, such compounds are expected to have CNS-activity.

Thus, in another aspect, provided are methods of treating a CNS-related disorder in a subject in need thereof, comprising administering to the subject an effective amount of a compound of the present invention. In certain embodiments, CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, or status epilepticus. In certain embodiments, the CNS-related disorder is depression. In certain embodiments, the CNS-related disorder is postpartum depression. In certain embodiments, the CNS-related disorder is major depressive disorder. In certain embodiments, the major depressive disorder is moderate major depressive disorder. In certain embodiments, the major depressive disorder is severe major depressive disorder. In certain embodiments, the compound is administered orally, subcutaneously, intravenously, or intramuscularly. In certain embodiments, the compound is administered orally. In certain embodiments, the compound is administered chronically. In certain embodiments, the compound is administered continuously, e.g., by continuous intravenous infusion.

Exemplary compounds of the invention may be synthesized from the following known starting materials using methods known to one skilled in the art or certain references, In one aspect, provided herein is a pharmaceutically acceptable salt of a compound described herein (e.g., a compound of Formulae (1-1), (2-1) or (3-1)).

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In one aspect, provided herein is a pharmaceutical composition comprising a compound described herein (e.g., a compound of Formulae (1-I), (2-I) or (3-I)) or a pharmaceutically acceptable salt thereof, and a pharmaceutically acceptable excipient. In certain embodiments, the compound of the present invention is provided in an effective amount in the pharmaceutical composition. In certain embodiments, the compound of the present invention is provided in a therapeutically effective amount.

In certain embodiments, the pharmaceutical composition comprises an effective amount of the active ingredient. In certain embodiments, the pharmaceutical composition comprises a therapeutically effective amount of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the onset of a CNS-disorder, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg of the compound provided herein, with preferred doses each providing from about 0.1 to about 10 mg/kg, and especially about 1 to about 5 mg/kg.

Transdermal doses are generally selected to provide similar or lower blood levels than are achieved using injection doses, generally in an amount ranging from about 0.01 to about 20% by weight, preferably from about 0.1 to about 20% by weight, preferably from about 0.1 to about 10% by weight, and more preferably from about 0.5 to about 15% by weight.

Injection dose levels range from about 0.1 mg/kg/hour to at least 20 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 5 g/day for a 40 to 80 kg human patient.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like.

Transdermal compositions are typically formulated as a topical ointment or cream containing the active ingredient(s). When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present invention can also be administered in sustained release forms or from sustained release drug delivery systems. A description of representative sustained release materials can be found in Remington's Pharmaceutical Sciences.

The present invention also relates to the pharmaceutically acceptable acid addition salt of a compound of the present invention. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

In another aspect, the invention provides a pharmaceutical composition comprising a compound of the present invention and a pharmaceutically acceptable excipient, e.g., a composition suitable for injection, such as for intravenous (IV) administration.

Pharmaceutically acceptable excipients include any and all diluents or other liquid vehicles, dispersion or suspension aids, surface active agents, isotonic agents, preservatives, lubricants and the like, as suited to the particular dosage form desired, e.g., injection. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

For example, injectable preparations, such as sterile injectable aqueous suspensions, can be formulated according to the known art using suitable dispersing or wetting agents and suspending agents. Exemplary excipients that can be employed include, but are not limited to, water, sterile saline or phosphate-buffered saline, or Ringer's solution.

In certain embodiments, the pharmaceutical composition further comprises a cyclodextrin derivative. The most common cyclodextrins are α-, β- and γ-cyclodextrins consisting of 6, 7 and 8 α-1,4-linked glucose units, respectively, optionally comprising one or more substituents on the linked sugar moieties, which include, but are not limited to, substituted or unsubstituted methylated, hydroxyalkylated, acylated, and sulfoalkylether substitution. In certain embodiments, the cyclodextrin is a sulfoalkyl ether β-cyclodextrin, e.g., for example, sulfobutyl ether β-cyclodextrin, also known as CAPTISOL®. See, e.g., U.S. Pat. No. 5,376,645. In certain embodiments, the composition comprises hexapropyl-β-cyclodextrin. In a more particular embodiment, the composition comprises hexapropyl-β-cyclodextrin (10-50% in water).

The injectable composition can be sterilized, for example, by filtration through a bacterial-retaining filter, or by incorporating sterilizing agents in the form of sterile solid compositions which can be dissolved or dispersed in sterile water or other sterile injectable medium prior to use.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, response of the individual patient, the severity of the patient's symptoms, and the like.

The compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pre-filled, pre-measured ampules or syringes of the liquid compositions. In such compositions, the compound is usually a minor component (from about 0.1% to about 50% by weight or preferably from about 1% to about 40% by weight) with the remainder being various vehicles or carriers and processing aids helpful for forming the desired dosing form.

The compounds provided herein can be administered as the sole active agent, or they can be administered in combination with other active agents. In one aspect, the present invention provides a combination of a compound of the present invention and another pharmacologically active agent. Administration in combination can proceed by any technique apparent to those of skill in the art including, for example, separate, sequential, concurrent, and alternating administration.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

In one aspect, provided is a kit comprising a composition (e.g., a solid composition) comprising a compound of Formulae (1-I), (2-I) or (3-I).

### Combination Therapy

A compound or composition described herein (e.g., a compound of Formulae (I-1), (1-2), (I-3) or (1-4), or a pharmaceutical salt thereof, or a composition comprising a compound of Formulae (I-1), (1-2), (1-3) or (I-4), or a pharmaceutically acceptable salt thereof) may be administered in combination with an additional agent or therapy. A subject to be administered a compound disclosed herein may have a disease, disorder, or condition, or a symptom thereof, that would benefit from treatment with another agent or therapy. Combination therapy may be achieved by administering two or more agents, each of which is formulated and administered separately, or by administering two or more agents in a single formulation. In some embodiments, the two or more agents in the combination therapy can be administered simultaneously. In other embodiments, the two or more agents in the combination therapy are administered separately. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X-Y-X, X-X-Y, Y-X-Y, Y-Y-X, X-X-Y-Y, etc. Exemplary additional agents are described below.

### Selective Serotonin Reuptake Inhibitor (SSRI)

In some embodiments, the compound or composition described herein (e.g., a compound of Formulae (I-1), (1-2), (1-3) or (1-4), or a pharmaceutical salt thereof, or a composition comprising a compound of Formulae (1-1), (1-2), (1-3) or (1-4), or a pharmaceutically acceptable salt thereof) is administered in combination with an SSRI(s). SSRIs include antidepressants that increase the level of serotonin in the brain. Exemplary SSRIs include, but are not limited to, Citalopram (Celexa), Escitalopram (Lexapro), Fluoxetine (Prozac), Fluvoxamine (Luvox), Paroxetine (Paxil), and Sertraline (Zoloft).

### Norepinephrine Reuptake Inhibitor (NERI)

In some embodiments, the compound or composition described herein (e.g., a compound of Formulae (I-1), (1-2), (1-3) or (1-4), or a pharmaceutical salt thereof, or a composition comprising a compound of Formulae (I-1), (I-2), (I-3) or (I-4), or a pharmaceutically acceptable salt thereof) is administered in combination with an NERI(s). Exemplary NERIs include, but are not limited to, Atomoxetine (Strattera), Reboxetine (Edronax, Vestra), Bupropion (Wellbutrin, Zyban), Duloxetine, Desipramine (Norpramin), Amedalin (UK-3540-1), Daledalin (UK-3557-15), Edivoxetine (LY-2216684), Esreboxetine, Lortalamine (LM-1404), Nisoxetine (LY-94,939), Talopram (tasulopram) (Lu 3-010), Talsupram (Lu 5-005), Tandamine (AY-23,946), and Viloxazine (Vivalan).

### Antipsychotics

In some embodiments, the compound or composition described herein (e.g., a compound of Formulae (I-1), (I-2), (I-3) or (I-4), or a pharmaceutical salt thereof, or a composition comprising a compound of Formulae (1-1), (1-2), (1-3) or (1-4), or a pharmaceutically acceptable salt thereof) is administered in combination with an antipsychotic agent(s). Antipsychotics include D2 antagonists, lowering dopaminergic neurotransmission in the dopamine pathways. Exemplary antipsychotics include, but are not limited to, Asenapine (Saphris), Aripiprazole (Abilify), Cariprazine (Vrayar), Clozapine (Clozaril), Droperidol, Fluperlapine, Mesoridazine, Quetiapine Hemifumarate, Raclopride, Spiperone, Sulpiride, Trimethobenzamide hydrochloride, Trifluoperazine Dihydrochloride, lurasidone (Latuda), Olanzapine (Zyprexa), Quetiapine (Seroquel), Zotepine, Risperidone (Risperdal), Ziprasidone (Geodon), Mesotidazine, Chlorpromazine hydrochloride, and Haloperidol (Haldol).

### Cannabinoids

In some embodiments, the compound or composition described herein (e.g., a compound of Formulae (I-1), (I-2), (I-3) or (I-4), or a pharmaceutical salt thereof, or a composition comprising a compound of Formulae (I-1), (1-2), (1-3) or (1-4), or a pharmaceutically acceptable salt thereof) is administered in combination with a cannabinoid(s). Exemplary cannabinoids include, but are not limited to, Cannabidiol (Epidiolex), Tetrahydrocannabinolic Acid, Tetrahydrocannabinol, Cannabidolic Acid, Cannabinol, Cannabigerol, Cannabichromene, Tetrahydrocannabivarin, and Cannabidivarin.

### AMDA Receptor Antagonists

In some embodiments, the compound or composition described herein (e.g., a compound of Formulae (I-1), (1-2), (1-3) or (I-4), or a pharmaceutical salt thereof, or a composition comprising a compound of Formulae (I-1), (1-2), (1-3) or (1-4), or a pharmaceutically acceptable salt thereof) is administered in combination with an NMDA receptor antagonist(s). NMDA receptor antagonists are a class of drugs that inhibit the action of the N-methyl-d-aspartate receptor. Exemplary NMDA antagonists include, but are not limited to, Ketamine, Esketamine, Ketobemidone, Ifendopril, 5,7-Dichlorokynurenic Acid, Licostinel, Memantine, Gavestinel, Phencyclidine, Dextromethorphan, Remacemide, Selfotel, Tiletamine, Dextropropoxyphene, Aptiganel, Dexanabinol, and Amantadine. NMDA receptor antagonists also include opioids such as Methadone, Dextropropoxyphene, Pethidine, Levorphanol, Tramadol, Neramexane, and Ketobemidone.

### GABA Receptor Agonists

In some embodiments, the compound or composition described herein (e.g., a compound of Formulae (T-1), (1-2), (1-3) or (1-4), or a pharmaceutical salt thereof, or a composition comprising a compound of Formulae (I-1), (I-2), (I-3) or (I-4), or a pharmaceutically acceptable salt thereof) is administered in combination with GABA receptor agaonist(s). GABA receptor agonist are a class of drugs that are agonists for one or more of the GABA receptors. Exemplary GABA receptor agonists include, but are not limited to, Clobazam, Topiramate, Muscimol, Progabide, Riluzole, Baclofen, Gabapentin, Vigabatrin, Valproic Acid, Tiagabine, Lamotrigine, Pregabalin, Phenyloin, Carbamazepine, Thiopental, Thiamylal, Pentobarbital, Secobarbital, Hexobarbital, Butobarbital, Amobarbital, Barbital, Mephobarbital, Phenobarbital, Primidone, Midazolam, Triazolam, Lometazepam, Flutazolam, Nitrazepam, Fluritrazepam, Nimetazepam, Diazepam, Medazepam, Oxazolam, Prazeam, Tofisopam, Rilmazafonoe, Lorazepam, Temazepam, Oxazepam, Fluidazepam, Chlordizaepoxide, Cloxazolam, Flutoprazepam, Alprazolam, Estazolam, Bromazepam, Flurazepam, Clorazepate Potassium, Haloxazolam, Ethyl Loflazepate, Qazepam, Clonazepam, Mexazolam, Etizolam, Brotizolam, Clotizaepam, Propofol, Fospropofol, Zolpidem, Zopiclone, Exzopiclone, Muscimol, TFQP/gaboxadol, Isoguvacine, Kojic amine, GABA, Homotaurine, Homohypotaurine, Trans-aminocyclopentane-3-carboxylic acid, Trans-amino-4-crotonic acid, b-guanidinopropionic acid, homo-b-proline, Isonipecotic acid, 3-((aminoiminomethyl)thio)-2-propenoic acid (ZAP A), Imidazoleacetic acid, and Piperidine-4-sulfonic acid (P4S).

### Cholinesterase Inhibitors

In some embodiments, the compound or composition described herein (e.g., a compound of Formulae (I-1), (1-2), (1-3) or (I-4), or a pharmaceutical salt thereof, or a composition comprising a compound of Formulae (I-1), (I-2), (I-3) or (I-4), or a pharmaceutically acceptable salt thereof) is administered in combination with a cholinesterase inhibitor(s). In general, cholinergics are compounds which mimic the action of acetylcholine and/or butyrylcholine. Cholinesterase inhibitors are a class of drugs that prevent the breakdown of acetylcholine. Exemplary cholinesterase inhibitors include, but are not limited to, Donepizil (Aricept), Tacrine (Cognex), Rivastigmine (Exelon, Exelon Patch), Galantamine (Razadyne, Reminyl), Memantine/Donepezil (Namzaric), Ambenonium (Mytelase), Neostigmine (Bloxiverz), Pyridostigmine (Mestinon Timespan, Regonol), and Galantamine (Razadyne).

The present disclosure also contemplates, among other things administration of a compound or pharmaceutical composition described herein (e.g., a compound of Formulae (I-1), (1-2), (I-3) or (1-4), or a pharmaceutical salt thereof, or a composition comprising a compound of Formulae (I-1), (1-2), (1-3) or (I-4), or a pharmaceutically acceptable salt thereof) to a subject has been previously administered an agent selected from the group consisting of a bronchial muscle/airway relaxant, an antiviral, oxygen, an antibody, and an antibacterial. In some embodiments an additional agent is administered to a subject prior to administration of a compound or pharmaceutical composition described herein (e.g., a compound of Formulae (I-1), (1-2), (1-3) or (I-4), or a pharmaceutical salt thereof, or a composition comprising a compound of Formulae (I-1), (1-2), (1-3) or (1-4), or a pharmaceutically acceptable salt thereof) and an additional agent is selected from the group consisting of a bronchial muscle/airway relaxant, an antiviral, oxygen, an antibody, and an antibacterial. In some embodiments, a compound or pharmaceutical composition described herein (e.g., a compound of Formulae (I-1), (1-2), (1-3) or (1-4), or a pharmaceutical salt thereof, or a composition comprising a compound of Formulae (I-1), (1-2), (1-3) or (I-4), or a pharmaceutically acceptable salt thereof) is co-administered with to a subject with an agent selected from a bronchial muscle/airway relaxant, an antiviral, oxygen, and an antibacterial

### Methods of Use and Treatment

In an aspect, compounds described herein, e.g., compounds of Formulae (1-I), (2-I) or (3-I), are envisioned to be useful as therapeutic agents for treating a CNS-related disorder (e.g., sleep disorder, a mood disorder such as depression, a schizophrenia spectrum disorder, a convulsive disorder, epileptogenesis, a disorder of memory and/or cognition, a movement disorder, a personality disorder, autism spectrum disorder, pain, traumatic brain injury, a vascular disease, a substance abuse disorder and/or withdrawal syndrome, or tinnitus) in a subject in need (e.g., a subject with Rett syndrome, Fragile X syndrome, or Angelman syndrome). Exemplary CNS conditions related to GABA-modulation include, but are not limited to, sleep disorders [e.g., insomnia], mood disorders [e.g., depression depression (e.g., major depressive disorder (MDD)), dysthymic disorder (e.g., mild depression), bipolar disorder (e.g., I and/or II), anxiety disorders (e.g., generalized anxiety disorder (GAD), social anxiety disorder), stress, post-traumatic stress disorder (PTSD), compulsive disorders (e.g., obsessive compulsive disorder (OCD))], schizophrenia spectrum disorders [e.g., schizophrenia, schizoaffective disorder], convulsive disorders [e.g., epilepsy (e.g., status epilepticus (SE)), seizures], disorders of memory and/or cognition [e.g., attention disorders (e.g., attention deficit hyperactivity disorder (ADHD)), dementia (e.g., Alzheimer's type dementia, Lewis body type dementia, vascular type dementia], movement disorders [e.g., Huntington's disease, Parkinson's disease], personality disorders [e.g., anti-social personality disorder, obsessive compulsive personality disorder], autism spectrum disorders (ASD) [e.g., autism, monogenetic causes of autism such as synaptophathy's, e.g., Rett syndrome, Fragile X syndrome, Angelman syndrome], pain [e.g., neuropathic pain, injury related pain syndromes, acute pain, chronic pain], traumatic brain injury (TBI), vascular diseases [e.g., stroke, ischemia, vascular malformations], substance abuse disorders and/or withdrawal syndromes [e.g., addition to opiates, cocaine, and/or alcohol], and tinnitus.

In certain embodiments, CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, or status epilepticus. In certain embodiments, the CNS-related disorder is depression. In certain embodiments, the CNS-related disorder is postpartum depression. In certain embodiments, the CNS-related disorder is major depressive disorder. In certain embodiments, the major depressive disorder is moderate major depressive disorder. In certain embodiments, the major depressive disorder is severe major depressive disorder.

In an aspect, provided is a method of alleviating or preventing seizure activity in a subject, comprising administering to the subject in need of such treatment an effective amount of a compound of the present invention. In some embodiments, the method alleviates or prevents epileptogenesis.

In yet another aspect, provided is a combination of a compound of the present invention and another pharmacologically active agent. The compounds provided herein can be administered as the sole active agent or they can be administered in combination with other agents. Administration in combination can proceed by any technique apparent to those of skill in the art including, for example, separate, sequential, concurrent and alternating administration.

In another aspect, provided is a method of treating or preventing brain excitability in a subject susceptible to or afflicted with a condition associated with brain excitability, comprising administering to the subject an effective amount of a compound of the present invention to the subject.

In yet another aspect, provided is a method of treating or preventing stress or anxiety in a subject, comprising administering to the subject in need of such treatment an effective amount of a compound of the present invention, or a composition thereof.

In yet another aspect, provided is a method of alleviating or preventing insomnia in a subject, comprising administering to the subject in need of such treatment an effective amount of a compound of the present invention, or a composition thereof.

In yet another aspect, provided is a method of inducing sleep and maintaining substantially the level of REM sleep that is found in normal sleep, wherein substantial rebound insomnia is not induced, comprising administering an effective amount of a compound of the present invention.

In yet another aspect, provided is a method of alleviating or preventing premenstrual syndrome (PMS) or postnatal depression (PND) in a subject, comprising administering to the subject in need of such treatment an effective amount of a compound of the present invention.

In yet another aspect, provided is a method of treating or preventing mood disorders in a subject, comprising administering to the subject in need of such treatment an effective amount of a compound of the present invention. In certain embodiments the mood disorder is depression.

In yet another aspect, provided is a method of cognition enhancement or treating memory disorder by administering to the subject a therapeutically effective amount of a compound of the present invention. In certain embodiments, the disorder is Alzheimer's disease. In certain embodiments, the disorder is Rett syndrome.

In yet another aspect, provided is a method of treating attention disorders by administering to the subject a therapeutically effective amount of a compound of the present invention. In certain embodiments, the attention disorder is ADHD.

In certain embodiments, the compound is administered to the subject chronically. In certain embodiments, the compound is administered to the subject orally, subcutaneously, intramuscularly, or intravenously.

### Neuroendocrine Disorders and Dysfunction

Provided herein are methods that can be used for treating neuroendocrine disorders and dysfunction. As used herein, “neuroendocrine disorder” or “neuroendocrine dysfunction” refers to a variety of conditions caused by imbalances in the body's hormone production directly related to the brain. Neuroendocrine disorders involve interactions between the nervous system and the endocrine system. Because the hypothalamus and the pituitary gland are two areas of the brain that regulate the production of hormones, damage to the hypothalamus or pituitary gland, e.g., by traumatic brain injury, may impact the production of hormones and other neuroendocrine functions of the brain. In some embodiments, the neuroendocrine disorder or dysfunction is associated with a women's health disorder or condition (e.g., a women's health disorder or condition described herein). In some embodiments, the neuroendocrine disorder or dysfunction is associated with a women's health disorder or condition is polycystic ovary syndrome.

Symptoms of neuroendocrine disorder include, but are not limited to, behavioral, emotional, and sleep-related symptoms, symptoms related to reproductive function, and somatic symptoms; including but not limited to fatigue, poor memory, anxiety, depression, weight gain or loss, emotional lability, lack of concentration, attention difficulties, loss of lipido, infertility, amenorrhea, loss of muscle mass, increased belly body fat, low blood pressure, reduced heart rate, hair loss, anemia, constipation, cold intolerance, and dry skin.

### Neurodegenerative Diseases and Disorders

The methods described herein can be used for treating neurodegenerative diseases and disorders. The term “neurodegenerative disease” includes diseases and disorders that are associated with the progressive loss of structure or function of neurons, or death of neurons. Neurodegenerative diseases and disorders include, but are not limited to, Alzheimer's disease (including the associated symptoms of mild, moderate, or severe cognitive impairment); amyotrophic lateral sclerosis (ALS); anoxic and ischemic injuries; ataxia and convulsion (including for the treatment and prevention and prevention of seizures that are caused by schizoaffective disorder or by drugs used to treat schizophrenia); benign forgetfulness; brain edema; cerebellar ataxia including McLeod neuroacanthocytosis syndrome (MLS); closed head injury; coma; contusive injuries (e.g., spinal cord injury and head injury); dementias including multi-infarct dementia and senile dementia; disturbances of consciousness; Down syndrome; drug-induced or medication-induced Parkinsonism (such as neuroleptic-induced acute akathisia, acute dystonia, Parkinsonism, or tardive dyskinesia, neuroleptic malignant syndrome, or medication-induced postural tremor); epilepsy; fragile X syndrome; Gilles de la Tourette's syndrome; head trauma; hearing impairment and loss; Huntington's disease; Lennox syndrome; levodopa-induced dyskinesia; mental retardation; movement disorders including akinesias and akinetic (rigid) syndromes (including basal ganglia calcification, corticobasal degeneration, multiple system atrophy, Parkinsonism-ALS dementia complex, Parkinson's disease, postencephalitic parkinsonism, and progressively supranuclear palsy); muscular spasms and disorders associated with muscular spasticity or weakness including chorea (such as benign hereditary chorea, drug-induced chorea, hemiballism, Huntington's disease, neuroacanthocytosis, Sydenham's chorea, and symptomatic chorea), dyskinesia (including tics such as complex tics, simple tics, and symptomatic tics), myoclonus (including generalized myoclonus and focal cyloclonus), tremor (such as rest tremor, postural tremor, and intention tremor) and dystonia (including axial dystonia, dystonic writer's cramp, hemiplegic dystonia, paroxysmal dystonia, and focal dystonia such as blepharospasm, oromandibular dystonia, and spasmodic dysphonia and torticollis); neuronal damage including ocular damage, retinopathy or macular degeneration of the eye; neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest; Parkinson's disease; seizure; status epilecticus; stroke; tinnitus; tubular sclerosis, and viral infection induced neurodegeneration (e.g., caused by acquired immunodeficiency syndrome (AIDS) and encephalopathies). Neurodegenerative diseases also include, but are not limited to, neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest. Methods of treating or preventing a neurodegenerative disease also include treating or preventing loss of neuronal function characteristic of neurodegenerative disorder.

### Mood Disorders

Also provided herein are methods for treating a mood disorder, for example clinical depression, postnatal depression or postpartum depression, perinatal depression, atypical depression, melancholic depression, psychotic major depression, cataonic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior. In some embodiments, the method described herein provides therapeutic effect to a subject suffering from depression (e.g., moderate or severe depression). In some embodiments, the mood disorder is associated with a disease or disorder described herein (e.g., neuroendocrine diseases and disorders, neurodegenerative diseases and disorders (e.g., epilepsy), movement disorders, tremor (e.g., Parkinson's Disease), women's health disorders or conditions).

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Peripartum depression refers to depression in pregnancy. Symptoms include irritability, crying, feeling restless, trouble sleeping, extreme exhaustion (emotional and/or physical), changes in appetite, difficulty focusing, increased anxiety and/or worry, disconnected feeling from baby and/or fetus, and losing interest in formerly pleasurable activities.

Postnatal depression (PND) is also referred to as postpartum depression (PPD), and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression, or a symptom of depression during pregnancy. This depression is referred to herein as) perinatal depression. In an embodiment, a subject experiencing perinatal depression is at increased risk of experiencing PND.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). During periods of mania the individual may feel or act abnormally happy, energetic, or irritable. They often make poorly thought out decisions with little regard to the consequences. The need for sleep is usually reduced. During periods of depression there may be crying, poor eye contact with others, and a negative outlook on life. The risk of suicide among those with the disorder is high at greater than 6% over 20 years, while self-harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or physchological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Post-surgical depression refers to feelings of depression that follow a surgical procedure (e.g., as a result of having to confront one's mortality). For example, individuals may feel sadness or empty mood persistently, a loss of pleasure or interest in hobbies and activities normally enjoyed, or a persistent felling of worthlessness or hopelessness.

Mood disorder associated with conditions or disorders of women's health refers to mood disorders (e.g., depression) associated with (e.g., resulting from) a condition or disorder of women's health (e.g., as described herein).

Suicidality, suicidal ideation, suicidal behavior refers to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case to case basis. Symptoms of depression, and relief of the same, may be ascertained by a physician or psychologist (e.g., by a mental state examination).

In some embodiments, the method comprises monitoring a subject with a known depression scale, e.g., the Hamilton Depression (HAM-D) scale, the Clinical Global Impression-Improvement Scale (CGI), and the Montgomery-Asberg Depression Rating Scale (MADRS). In some embodiments, a therapeutic effect can be determined by reduction in Hamilton Depression (HAM-D) total score exhibited by the subject. Reduction in the HAM-D total score can happen within 4, 3, 2, or 1 days; or 96, 84, 72, 60, 48, 24, 20, 16, 12, 10, 8 hours or less. The therapeutic effect can be assessed across a specified treatment period. For example, the therapeutic effect can be determined by a decrease from baseline in HAM-D total score after administering a compound described herein, e.g., a compound of Formulae (1-I), (2-I) or (3-I) (e.g., 12, 24, or 48 hours after administration; or 24, 48, 72, or 96 hours or more; or 1 day, 2 days, 14 days, 21 days, or 28 days; or 1 week, 2 weeks, 3 weeks, or 4 weeks; or 1 month, 2 months, 6 months, or 10 months; or 1 year, 2 years, or for life).

In some embodiments, the subject has a mild depressive disorder, e.g., mild major depressive disorder. In some embodiments, the subject has a moderate depressive disorder, e.g., moderate major depressive disorder. In some embodiments, the subject has a severe depressive disorder, e.g., severe major depressive disorder. In some embodiments, the subject has a very severe depressive disorder, e.g., very severe major depressive disorder. In some embodiments, the baseline HAM-D total score of the subject (i.e., prior to treatment with a compound described herein, e.g., a compound of Formulae (1-I), (2-I) or (3-I)) is at least 24. In some embodiments, the baseline HAM-D total score of the subject is at least 18. In some embodiments, the baseline HAM-D total score of the subject is between and including 14 and 18. In some embodiments, the baseline HAM-D total score of the subject is between and including 19 and 22. In some embodiments, the HAM-D total score of the subject before treatment with a compound described herein, e.g., a compound of Formulae (1-I), (2-I) or (3-I), is greater than or equal to 23. In some embodiments, the baseline score is at least 10, 15, or 20. In some embodiments, the HAM-D total score of the subject after treatment with a compound described herein, e.g., a compound of Formulae (1-I), (2-I) or (3-I), is about 0 to 10 (e.g., less than 10; 0 to 10, 0 to 6, 0 to 4, 0 to 3, 0 to 2, or 1.8). In some embodiments, the HAM-D total score after treatment with a compound described herein, e.g., a compound of Formulae (1-I), (2-I) or (3-I), is less than 10, 7, 5, or 3. In some embodiments, the decrease in HAM-D total score is from a baseline score of about 20 to 30 (e.g., 22 to 28, 23 to 27, 24 to 27, 25 to 27, 26 to 27) to a HAM-D total score at about 0 to 10 (e.g., less than 10; 0 to 10, 0 to 6, 0 to 4, 0 to 3, 0 to 2, or 1.8) after treatment with a compound described herein, e.g., a compound of Formulae (1-I), (2-I) or (3-I). In some embodiments, the decrease in the baseline HAM-D total score to HAM-D total score after treatment with a compound described herein, e.g., a compound of Formulae (1-1), (2-1) or (3-I), is at least 1, 2, 3, 4, 5, 7, 10, 25, 40, 50, or 100 fold). In some embodiments, the percentage decrease in the baseline HAM-D total score to HAM-D total score after treatment with a compound described herein, e.g., a compound of Formulae (1-I), (2-I) or (3-I), is at least 50% (e.g., 60%, 70%, 80%, or 90%). In some embodiments, the therapeutic effect is measured as a decrease in the HAM-D total score after treatment with a compound described herein, e.g., a compound of Formulae (1-I), (2-I) or (3-I), relative to the baseline HAM-D total score (e.g., 12, 24, 48 hours after administration; or 24, 48, 72, 96 hours or more; or 1 day, 2 days, 14 days, or more) is at least 10, 15, or 20 points.

In some embodiments, the method of treating a depressive disorder, e.g., major depressive disorder provides a therapeutic effect (e.g., as measured by reduction in Hamilton Depression Score (HA1-D)) within 14, 10, 4, 3, 2, or 1 days, or 24, 20, 16, 12, 10, or 8 hours or less. In some embodiments, the method of treating the depressive disorder, e.g., major depressive disorder, provides a therapeutic effect (e.g., as determined by a statistically significant reduction in HAM-D total score) within the first or second day of the treatment with a compound described herein, e.g., a compound of Formulae (1-I), (2-I) or (3-I). In some embodiments, the method of treating the depressive disorder, e.g., major depressive disorder, provides a therapeutic effect (e.g., as determined by a statistically significant reduction in HAM-D total score) within less than or equal to 14 days since the beginning of the treatment with a compound described herein, e.g., a compound of Formulae (1-1), (2-1) or (3-I). In some embodiments, the method of treating the depressive disorder, e.g., major depressive disorder, provides a therapeutic effect (e.g., as determined by a statistically significant reduction in HAM-D total score) within less than or equal to 21 days since the beginning of the treatment with a compound described herein, e.g., a compound of Formulae (1-I), (2-I) or (3-I). In some embodiments, the method of treating the depressive disorder, e.g., major depressive disorder, provides a therapeutic effect (e.g., as determined by a statistically significant reduction in HAM-D total score) within less than or equal to 28 days since the beginning of the treatment with a compound described herein, e.g., a compound of Formulae (1-I), (2-I) or (3-I). In some embodiments, the therapeutic effect is a decrease from baseline in HAM-D total score after treatment with a compound described herein, e.g., a compound of Formulae (1-I), (2-I) or (3-I) (e.g., treatment with a compound described herein, e.g., a compound of Formulae (1-I), (2-I) or (3-I), once a day for 14 days). In some embodiments, the HAM-D total score of the subject before treatment with a compound described herein, e.g., a compound of Formulae (1-I), (2-I) or (3-I), is at least 24. In some embodiments, the HAM-D total score of the subject before treatment with a compound described herein, e.g., a compound of Formulae (1-I), (2-I) or (3-I), is at least 18. In some embodiments, the HAM-D total score of the subject before treatment with a compound described herein, e.g., a compound of Formulae (1-I), (2-I) or (3-I), is between and including 14 and 18. In some embodiments, the decrease in HAM-D total score after treating the subject with a compound described herein, e.g., a compound of Formulae (1-I), (2-I) or (3-I), relative to the baseline HAVI-D total score is at least 10. In some embodiments, the decrease in HAM-D total score after treating the subject with a compound described herein, e.g., a compound of Formulae (1-I), (2-I) or (3-I), relative to the baseline HAM-D total score is at least 15 (e.g., at least 17). In some embodiments, the HAM/I-D total score associated with treating the subject with a compound described herein, e.g., a compound of Formulae (1-I), (2-I) or (3-I), is no more than a number ranging from 6 to 8. In some embodiments, the HAM-D total score associated with treating the subject with a compound described herein, e.g., a compound of Formulae (1-I), (2-I) or (3-I), is no more than 7.

In some embodiments, the method provides therapeutic effect (e.g., as measured by reduction in Clinical Global Impression-Improvement Scale (CGI)) within 14, 10, 4, 3, 2, or 1 days, or 24, 20, 16, 12, 10, or 8 hours or less. In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, the therapeutic effect is a decrease from baseline in CGI score at the end of a treatment period (e.g., 14 days after administration).

In some embodiments, the method provides therapeutic effect (e.g., as measured by reduction in Montgomery-Asberg Depression Rating Scale (MADRS)) within 14, 10, 4, 3, 2, or 1 days, or 24, 20, 16, 12, 10, or 8 hours or less. In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, the therapeutic effect is a decrease from baseline in MADRS score at the end of a treatment period (e.g., 14 days after administration).

A therapeutic effect for major depressive disorder can be determined by a reduction in Montgomery-Asberg Depression Rating Scale (MADRS) score exhibited by the subject. For example, the MADRS score can be reduced within 4, 3, 2, or 1 days; or 96, 84, 72, 60, 48, 24, 20, 16, 12, 10, 8 hours or less. The Montgomery-Asberg Depression Rating Scale (MADRS) is a ten-item diagnostic questionnaire (regarding apparent sadness, reported sadness, inner tension, reduced sleep, reduced appetite, concentration difficulties, lassitude, inability to feel, pessimistic thoughts, and suicidal thoughts) which psychiatrists use to measure the severity of depressive episodes in patients with mood disorders.

In some embodiments, the method provides therapeutic effect (e.g., as measured by reduction in Edinburgh Postnatal Depression Scale (EPDS)) within 4, 3, 2, 1 days; 24, 20, 16, 12, 10, 8 hours or less. In some embodiments, the therapeutic effect is an improvement measured by the EPDS.

In some embodiments, the method provides therapeutic effect (e.g., as measured by reduction in Generalized Anxiety Disorder 7-Item Scale (GAD-7)) within 4, 3, 2, 1 days; 24, 20, 16, 12, 10, 8 hours or less.

### Anxiety Disorders

Provided herein are methods for treating anxiety disorders (e.g., generalized anxiety disorder, panic disorder, obsessive compulsive disorder, phobia, post-traumatic stress disorder). Anxiety disorder is a blanket term covering several different forms of abnormal and pathological fear and anxiety. Current psychiatric diagnostic criteria recognize a wide variety of anxiety disorders.

Generalized anxiety disorder is a common chronic disorder characterized by long-lasting anxiety that is not focused on any one object or situation. Those suffering from generalized anxiety experience non-specific persistent fear and worry and become overly concerned with everyday matters. Generalized anxiety disorder is the most common anxiety disorder to affect older adults.

In panic disorder, a person suffers from brief attacks of intense terror and apprehension, often marked by trembling, shaking, confusion, dizziness, nausea, difficulty breathing. These panic attacks, defined by the APA as fear or discomfort that abruptly arises and peaks in less than ten minutes, can last for several hours and can be triggered by stress, fear, or even exercise; although the specific cause is not always apparent. In addition to recurrent unexpected panic attacks, a diagnosis of panic disorder also requires that said attacks have chronic consequences: either worry over the attacks' potential implications, persistent fear of future attacks, or significant changes in behavior related to the attacks. Accordingly, those suffering from panic disorder experience symptoms even outside of specific panic episodes. Often, normal changes in heartbeat are noticed by a panic sufferer, leading them to think something is wrong with their heart or they are about to have another panic attack. In some cases, a heightened awareness (hypervigilance) of body functioning occurs during panic attacks, wherein any perceived physiological change is interpreted as a possible life threatening illness (i.e. extreme hypochondriasis).

Obsessive compulsive disorder is a type of anxiety disorder primarily characterized by repetitive obsessions (distressing, persistent, and intrusive thoughts or images) and compulsions (urges to perform specific acts or rituals). The OCD thought pattern may be likened to superstitions insofar as it involves a belief in a causative relationship where, in reality, one does not exist. Often the process is entirely illogical; for example, the compulsion of walking in a certain pattern may be employed to alleviate the obsession of impending harm. And in many cases, the compulsion is entirely inexplicable, simply an urge to complete a ritual triggered by nervousness. In a minority of cases, sufferers of OCD may only experience obsessions, with no overt compulsions; a much smaller number of sufferers experience only compulsions.

The single largest category of anxiety disorders is that of phobia, which includes all cases in which fear and anxiety is triggered by a specific stimulus or situation. Sufferers typically anticipate terrifying consequences from encountering the object of their fear, which can be anything from an animal to a location to a bodily fluid.

Post-traumatic stress disorder or PTSD is an anxiety disorder which results from a traumatic experience. Post-traumatic stress can result from an extreme situation, such as combat, rape, hostage situations, or even serious accident. It can also result from long term (chronic) exposure to a severe stressor, for example soldiers who endure individual battles but cannot cope with continuous combat. Common symptoms include flashbacks, avoidant behaviors, and depression.

### Women's Health Disorders

Provided herein are methods for treating conditions or disorders related to women's health. Conditions or disorders related to women's health include, but are not limited to, gynecological health and disorders (e.g., premenstrual syndrome (PMS), premenstrual dysphoric disorder (PMDD)), pregnancy issues (e.g., miscarriage, abortion), infertility and related disorders (e.g., polycystic ovary syndrome (PCOS)), other disorders and conditions, and issues related to women's overall health and wellness (e.g., menopause).

Gynecological health and disorders affecting women include menstruation and menstrual irregularities; urinary tract health, including urinary incontinence and pelvic floor disorders; and such disorders as bacterial vaginosis, vaginitis, uterine fibroids, and vulvodynia.

Premenstrual syndrome (PMS) refers to physical and emotional symptoms that occur in the one to two weeks before a women's period. Symptoms vary but can include bleeding, mood swings, tender breasts, food cravings, fatigue, irritability, acne, and depression.

Premenstrual dysphoric disorder (PMDD) is a severe form of PMS. The symptoms of PMDD are similar to PMS but more severe and may interfere with work, social activity, and relationships. PMDD symptoms include mood swings, depressed mood or feelings of hopelessness, marked anger, increased interpersonal conflicts, tension and anxiety, irritability, decreased interest in usual activities, difficulty concentrating, fatigue, change in appetite, feeling out of control or overwhelmed, sleep problems, physical problems (e.g., bloating, breast tenderness, swelling, headaches, joint or muscle pain).

Pregnancy issues include preconception care and prenatal care, pregnancy loss (miscarriage and stillbirth), preterm labor and premature birth, sudden infant death syndrome (SIDS), breastfeeding, and birth defects.

Miscarriage refers to a pregnancy that ends on its own, within the first 20 weeks of gestation.

Abortion refers to the deliberate termination of a pregnancy, which can be performed during the first 28 weeks of pregnancy.

Infertility and related disorders include uterine fibroids, polycystic ovary syndrome, endometriosis, and primary ovarian insufficiency.

Polycystic ovary syndrome (PCOS) refers to an endocrine system disorder among women of reproductive age. PCOS is a set of symptoms resulting from an elevated male hormone in women. Most women with PCOS grow many small cysts on their ovaries. Symptoms of PCOS include irregular or no menstrual periods, heavy periods, excess body and facial hair, acne, pelvic pain, difficulty getting pregnant, and patches of thick, darker, velvety skin. PCOS may be associated with conditions including type 2 diabetes, obesity, obstructive sleep apnea, heart disease, mood disorders, and endometrial cancer.

Other disorders and conditions that affect only women include Turner syndrome, Rett syndrome, and ovarian and cervical cancers.

Issues related to women's overall health and wellness include violence against women, women with disabilities and their unique challenges, osteoporosis and bone health, and menopause.

Menopause refers to the 12 months after a woman's last menstrual period and marks the end of menstrual cycles. Menopause typically occurs in a woman's 40s or 50s. Physical symptoms such as hot flashes and emotional symptoms of menopause may disrupt sleep, lower energy, or trigger anxiety or feelings of sadness or loss. Menopause includes natural menopause and surgical menopause, which is a type of induced menopause due to an event such as surgery (e.g., hysterectomy, oophorectomy; cancer). It is induced when the ovaries are gravely damaged by, e.g., radiation, chemotherapy, or other medications.

### Epilepsy

The compound of Formulae (1-1), (2-1) or (3-I), or pharmaceutically acceptable salt, or a pharmaceutically acceptable composition thereof, can be used in a method described herein, for example in the treatment of a disorder described herein such as epilepsy, status epilepticus, or seizure.

Epilepsy is a brain disorder characterized by repeated seizures over time. Types of epilepsy can include, but are not limited to generalized epilepsy, e.g., childhood absence epilepsy, juvenile nyoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

### Epileptogenesis

The compounds and methods described herein can be used to treat or prevent epileptogenesis. Epileptogenesis is a gradual process by which a normal brain develops epilepsy (a chronic condition in which seizures occur). Epileptogenesis results from neuronal damage precipitated by the initial insult (e.g., status epilepticus).

Status epilepticus (SE)

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

The compound of Formulae (1-I), (2-I) or (3-I) or pharmaceutically acceptable salt, or a pharmaceutically acceptable composition thereof, can also be administered as a prophylactic to a subject having a CNS disorder e.g., a traumatic brain injury, status epilepticus, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges; prior to the onset of a seizure.

### Seizure

A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus.

There are six types of generalized seizures. The most common and dramatic, and therefore the most well-known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time.

Tonic seizures are characterized by stiffening of the muscles.

Atonic seizures consist of a sudden and general loss of muscle tone, particularly in the arms and legs, which often results in a fall.

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures. In some embodiments, the seizure is a generalized seizure associated with Dravet Syndrome, Lennox-Gastaut Syndrome, Tuberous Sclerosis Complex, Rett Syndrome or PCDH19 Female Pediatric Epilepsy.

### Movement Disorders

Also described herein are methods for treating a movement disorder. As used herein, “movement disorders” refers to a variety of diseases and disorders that are associated with hyperkinetic movement disorders and related abnormalities in muscle control. Exemplary movement disorders include, but are not limited to, Parkinson's disease and parkinsonism (defined particularly by bradykinesia), dystonia, chorea and Huntington's disease, ataxia, tremor (e.g., essential tremor), myoclonus and startle, tics and Tourette syndrome, Restless legs syndrome, stiff person syndrome, and gait disorders.

### Tremor

The methods described herein can be used to treat tremor, for example the compound of Formulae (1-I), (2-1) or (3-I) can be used to treat cerebellar tremor or intention tremor, dystonic tremor, essential tremor, orthostatic tremor, parkinsonian tremor, physiological tremor, psychogenic tremor, or rubral tremor. Tremor includes hereditary, degenerative, and idiopathic disorders such as Wilson's disease, Parkinson's disease, and essential tremor, respectively; metabolic diseases (e.g., thyroid-parathyroid-, liver disease and hypoglycemia); peripheral neuropathies (associated with Charcot-Marie-Tooth, Roussy-Levy, diabetes mellitus, complex regional pain syndrome); toxins (nicotine, mercury, lead, CO, Manganese, arsenic, toluene); drug-induced (narcoleptics, tricyclics, lithium, cocaine, alcohol, adrenaline, bronchodilators, theophylline, caffeine, steroids, valproate, amiodarone, thyroid hormones, vincristine); and psychogenic disorders. Clinical tremor can be classified into physiologic tremor, enhanced physiologic tremor, essential tremor syndromes (including classical essential tremor, primary orthostatic tremor, and task- and position-specific tremor), dystonic tremor, parkinsonian tremor, cerebellar tremor, Holmes' tremor (i.e., rubral tremor), palatal tremor, neuropathic tremor, toxic or drug-induced tremor, and psychogenic tremor.

Tremor is an involuntary, at times rhythmic, muscle contraction and relaxation that can involve oscillations or twitching of one or more body parts (e.g., hands, arms, eyes, face, head, vocal folds, trunk, legs).

Cerebellar tremor or intention tremor is a slow, broad tremor of the extremities that occurs after a purposeful movement. Cerebellar tremor is caused by lesions in or damage to the cerebellum resulting from, e.g., tumor, stroke, disease (e.g., multiple sclerosis, an inherited degenerative disorder).

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occurs irregularly and often can be relieved by complete rest.

Essential tremor or benign essential tremor is the most common type of tremor. Essential tremor may be mild and nonprogressive in some, and may be slowly progressive, starting on one side of the body but affect both sides within 3 years. The hands are most often affected, but the head, voice, tongue, legs, and trunk may also be involved. Tremor frequency may decrease as the person ages, but severity may increase. Heightened emotion, stress, fever, physical exhaustion, or low blood sugar may trigger tremors and/or increase their severity. Symptoms generally evolve over time and can be both visible and persistent following onset.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occurs in patients with essential tremor.

Parkinsonian tremor is caused by damage to structures within the brain that control movement. Parkinsonian tremor is often a precursor to Parkinson's disease and is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Physiological tremor can occur in normal individuals and have no clinical significance. It can be seen in all voluntary muscle groups. Physiological tremor can be caused by certain drugs, alcohol withdrawal, or medical conditions including an overactive thyroid and hypoglycemia. The tremor classically has a frequency of about 10 Hz.

Psychogenic tremor or hysterical tremor can occur at rest or during postural or kinetic movement. Patient with psychogenic tremor may have a conversion disorder or another psychiatric disease.

Rubral tremor is characterized by coarse slow tremor which can be present at rest, at posture, and with intention. The tremor is associated with conditions that affect the red nucleus in the midbrain, classical unusual strokes.

Parkinson's Disease affects nerve cells in the brain that produce dopamine. Symptoms include muscle rigidity, tremors, and changes in speech and gait. Parkinsonism is characterized by tremor, bradykinesia, rigidity, and postural instability. Parkinsonism shares symptoms found in Parkinson's Disease, but is a symptom complex rather than a progressive neurodegenerative disease.

Dystonia is a movement disorder characterized by sustained or intermittent muscle contractions causing abnormal, often repetitive movements or postures. Dystonic movements can be patterned, twisting, and may be tremulous. Dystonia is often initiated or worsened by voluntary action and associated with overflow muscle activation.

Chorea is a neurological disorder characterized by jerky involuntary movements typically affecting the shoulders, hips, and face. Huntington's Disease is an inherited disease that causes nerve cells in the brain to waste away. Symptoms include uncontrolled movements, clumsiness, and balance problems. Huntington's disease can hinder walk, talk, and swallowing.

Ataxia refers to the loss of full control of bodily movements, and may affect the fingers, hands, arms, legs, body, speech, and eye movements.

Myloclonus and Startle is a response to a sudden and unexpected stimulus, which can be acoustic, tactile, visual, or vestibular.

Tics are an involuntary movement usually onset suddenly, brief, repetitive, but non-rhythmical, typically imitating normal behavior and often occurring out of a background of normal activity. Tics can be classified as motor or vocal, motor tics associated with movements while vocal tics associated with sound. Tics can be characterized as simple or complex. For example simple motor tics involve only a few muscles restricted to a specific body part. Tourette Syndrome is an inherited neuropsychiatric disorder with onset in childhood, characterized by multiple motor tics and at least one vocal tic.

Restless Legs Syndrome is a neurologic sensorimotor disorder characterized by an overwhelming urge to move the legs when at rest.

Stiff Person Syndrome is a progressive movement disorder characterized by involuntary painful spasms and rigidity of muscles, usually involving the lower back and legs. Stiff-legged gait with exaggerated lumbar hyperlordosis typically results. Characteristic abnormality on EMG recordings with continuous motor unit activity of the paraspinal axial muscles is typically observed. Variants include “stiff-limb syndrome” producing focal stiffness typically affecting distal legs and feet.

Gait disorders refer to an abnormality in the manner or style of walking, which results from neuromuscular, arthritic, or other body changes. Gait is classified according to the system responsible for abnormal locomotion, and include hemiplegic gait, diplegic gait, neuropathic gait, myopathic gait, parkinsonian gait, choreiform gait, ataxic gait, and sensory gait.

### Anesthesia/Sedation

Anesthesia is a pharmacologically induced and reversible state of amnesia, analgesia, loss of responsiveness, loss of skeletal muscle reflexes, decreased stress response, or all of these simultaneously. These effects can be obtained from a single drug which alone provides the correct combination of effects, or occasionally with a combination of drugs (e.g., hypnotics, sedatives, paralytics, analgesics) to achieve very specific combinations of results. Anesthesia allows patients to undergo surgery and other procedures without the distress and pain they would otherwise experience.

Sedation is the reduction of irritability or agitation by administration of a pharmacological agent, generally to facilitate a medical procedure or diagnostic procedure.

Sedation and analgesia include a continuum of states of consciousness ranging from minimal sedation (anxiolysis) to general anesthesia.

Minimal sedation is also known as anxiolysis. Minimal sedation is a drug-induced state during which the patient responds normally to verbal commands. Cognitive function and coordination may be impaired. Ventilatory and cardiovascular functions are typically unaffected.

Moderate sedation/analgesia (conscious sedation) is a drug-induced depression of consciousness during which the patient responds purposefully to verbal command, either alone or accompanied by light tactile stimulation. No interventions are usually necessary to maintain a patent airway. Spontaneous ventilation is typically adequate. Cardiovascular function is usually maintained.

Deep sedation/analgesia is a drug-induced depression of consciousness during which the patient cannot be easily aroused, but responds purposefully (not a reflex withdrawal from a painful stimulus) following repeated or painful stimulation. Independent ventilatory function may be impaired and the patient may require assistance to maintain a patent airway. Spontaneous ventilation may be inadequate. Cardiovascular function is usually maintained.

General anesthesia is a drug-induced loss of consciousness during which the patient is not arousable, even to painful stimuli. The ability to maintain independent ventilatory function is often impaired and assistance is often required to maintain a patent airway. Positive pressure ventilation may be required due to depressed spontaneous ventilation or drug-induced depression of neuromuscular function. Cardiovascular function may be impaired.

Sedation in the intensive care unit (ICU) allows the depression of patients' awareness of the environment and reduction of their response to external stimulation. It can play a role in the care of the critically ill patient, and encompasses a wide spectrum of symptom control that will vary between patients, and among individuals throughout the course of their illnesses. Heavy sedation in critical care has been used to facilitate endotracheal tube tolerance and ventilator synchronization, often with neuromuscular blocking agents.

In some embodiments, sedation (e.g., long-term sedation, continuous sedation) is induced and maintained in the ICU for a prolonged period of time (e.g., 1 day, 2 days, 3 days, 5 days, 1 week, 2 week, 3 weeks, 1 month, 2 months). Long-term sedation agents may have long duration of action. Sedation agents in the ICU may have short elimination half-life.

Procedural sedation and analgesia, also referred to as conscious sedation, is a technique of administering sedatives or dissociative agents with or without analgesics to induce a state that allows a subject to tolerate unpleasant procedures while maintaining cardiorespiratory function.

Also described herein are methods of ameliorating one or more symptoms of a respiratory condition in a subject, comprising administering to the subject an effective amount of a compound or pharmaceutical composition described herein (e.g., a compound of Formulae (I-1), (I-2), (I-3) or (1-4), or a pharmaceutical salt thereof, or a composition comprising a compound of Formulae (I-1), (I-2), (1-3) or (1-4), or a pharmaceutically acceptable salt thereof).

In one aspect, provided herein is a method of treating a subject wherein the subject exhibits one or more symptoms of a respiratory condition and/or has been diagnosed with a respiratory condition, comprising administering to said subject an effective amount of a compound or pharmaceutical composition described herein (e.g., a compound of Formulae (I-1), (I-2), (I-3) or (1-4), or a pharmaceutical salt thereof, or a composition comprising a compound of Formulae (I-1), (I-2), (I-3) or (I-4), or a pharmaceutically acceptable salt thereof).

In some embodiments, the present disclosure contemplates a method of treating a subject comprising administering to said subject a compound or pharmaceutical composition described herein (e.g., a compound of Formulae (I-1), (I-2), (I-3) or (I-4), or a pharmaceutical salt thereof, or a composition comprising a compound of Formulae (I-1), (1-2), (I-3) or (I-4), or a pharmaceutically acceptable salt thereof), wherein the subject has a respiratory condition.

In some embodiments, administration of a compound or pharmaceutical composition described herein (e.g., a compound of Formulae (I-1), (1-2), (I-3) or (1-4), or a pharmaceutical salt thereof, or a composition comprising a compound of Formulae (I-1), (I-2), (1-3) or (1-4), or a pharmaceutically acceptable salt thereof) to a subject exhibiting symptoms of a respiratory condition, may result in the reduction of the severity of one or more symptoms of a respiratory condition or retard or slow the progression of one or more symptoms of a respiratory condition.

In some embodiments, a subject with a respiratory condition has been or is being treated with mechanical ventilation or oxygen. In some embodiments, a subject with a respiratory condition has been or is being treated with mechanical ventilation.

In some embodiments, a compound or pharmaceutical composition described herein (e.g., a compound of Formulae (I-1), (I-2), (I-3) or (1-4), or a pharmaceutical salt thereof, or a composition comprising a compound of Formulae (I-1), (1-2), (1-3) or (I-4), or a pharmaceutically acceptable salt thereof) is administered to a subject that is being or has been treated with mechanical ventilation. In some embodiments, administration of a compound or pharmaceutical composition described herein (e.g., a compound of Formulae (I-1), (I-2), (1-3) or (1-4), or a pharmaceutical salt thereof, or a composition comprising a compound of Formulae (I-1), (I-2), (I-3) or (1-4), or a pharmaceutically acceptable salt thereof) continues throughout a subject's treatment with mechanical ventilation. In some embodiments, administration of a compound or pharmaceutical composition described herein (e.g., a compound of Formulae (I-1), (I-2), (I-3) or (I-4), or a pharmaceutical salt thereof, or a composition comprising a compound of Formulae (I-1), (1-2), (1-3) or (1-4), or a pharmaceutically acceptable salt thereof) continues after a subject has ended treatment with mechanical ventilation.

In some embodiments, a compound or pharmaceutical composition described herein (e.g., a compound of Formulae (I-1), (I-2), (I-3) or (1-4), or a pharmaceutical salt thereof, or a composition comprising a compound of Formulae (I-1), (1-2), (1-3) or (I-4), or a pharmaceutically acceptable salt thereof) is administered to a subject who is receiving or has received treatment with a sedative. In some embodiments, a sedative is propofol or a benzodiazepine.

In some embodiments, the present disclosure includes administering to a subject in need thereof a compound or pharmaceutical composition described herein (e.g., a compound of Formulae (I-1), (I-2), (I-3) or (1-4), or a pharmaceutical salt thereof, or a composition comprising a compound of Formulae (I-1), (1-2), (1-3) or (1-4), or a pharmaceutically acceptable salt thereof) in an amount sufficient to increase oxygen saturation in blood. In some embodiments, oxygen saturation in blood is measured using pulse oximetry.

In some embodiments, the present disclosure contemplates a method of treating a cytokine storm in a patient. In some embodiments a method of treating a cytokine storm comprising the step of administering to the patient a compound or pharmaceutical composition described herein (e.g., a compound of Formulae (I-1), (1-2), (1-3) or (1-4), or a pharmaceutical salt thereof, or a composition comprising a compound of Formulae (I-1), (I-2), (1-3) or (1-4), or a pharmaceutically acceptable salt thereof). In some embodiments, a symptom of a cytokine storm is lung inflammation. In some embodiments, a patient undergoing a cytokine storm has acute respiratory distress syndrome (ARDS).

### Respiratory Condition

In some embodiments, a subject with a respiratory condition suffers from respiratory distress. In some embodiments, respiratory distress includes acute respiratory distress.

In some embodiments, a subject with a respiratory condition may exhibit one or more symptoms selected from the group consisting of airway hyper-responsiveness, inflammation of lung tissue, lung hypersensitivity, and inflammation-related pulmonary pain.

In some embodiments a subject with a respiratory condition may exhibit inflammation of lung tissue. In some embodiments, inflammation of lung tissue is bronchitis or bronchiectasis. In some embodiments, inflammation of lung tissue is pneumonia. In some embodiments, pneumonia is ventilator-associated pneumonia or hospital-acquired pneumonia. In some embodiments, pneumonia is ventilator-associated pneumonia.

In some embodiments, administration of the compound or pharmaceutical composition described herein to a subject exhibiting symptoms of a respiratory condition, results in reduction of the severity of respiratory distress in a subject with a respiratory condition or retard or slow the progression of respiratory distress in a subject with a respiratory condition.

In some embodiments, administration of a compound or pharmaceutical composition described herein (e.g., a compound of Formulae (I-1), (1-2), (1-3) or (1-4), or a pharmaceutical salt thereof, or a composition comprising a compound of Formulae (I-1), (I-2), (1-3) or (1-4), or a pharmaceutically acceptable salt thereof) to a subject exhibiting symptoms of a respiratory condition, results in reduction of the severity of airway hyper-responsiveness in a subject with a disease associated with a coronavirus or retard or slow the progression of airway hyper-responsiveness in a subject with a respiratory condition.

In some embodiments, administration of a compound or pharmaceutical composition described herein (e.g., a compound of Formulae (I-1), (1-2), (1-3) or (1-4), or a pharmaceutical salt thereof, or a composition comprising a compound of Formulae (I-1), (I-2), (1-3) or (1-4), or a pharmaceutically acceptable salt thereof) to a subject exhibiting symptoms of a respiratory condition, results in reduction of the severity of inflammation of lung tissue in a subject with a respiratory condition or retard or slow the progression of inflammation of lung tissue in a subject with a respiratory condition. In some embodiments, administration of a compound or pharmaceutical composition described herein (e.g., a compound of Formulae (I-1), (I-2), (I-3) or (1-4), or a pharmaceutical salt thereof, or a composition comprising a compound of Formulae (I-1), (1-2), (1-3) or (1-4), or a pharmaceutically acceptable salt thereof) to a subject exhibiting symptoms of a respiratory condition, results in reduction of the severity of pneumonia in a subject with a respiratory condition or retard or slow the progression of pneumonia in a subject with a respiratory condition.

In some embodiments, administration of a compound or pharmaceutical composition described herein (e.g., a compound of Formulae (I-1), (1-2), (1-3) or (1-4), or a pharmaceutical salt thereof, or a composition comprising a compound of Formulae (I-1), (I-2), (1-3) or (1-4), or a pharmaceutically acceptable salt thereof) to a subject exhibiting symptoms of a respiratory condition, results in reduction of the severity of lung hypersensitivity in a subject with a respiratory condition or retard or slow the progression of lung hypersensitivity in a subject with a respiratory condition.

In some embodiments, administration of a compound or pharmaceutical composition described herein (e.g., a compound of Formulae (I-1), (1-2), (1-3) or (1-4), or a pharmaceutical salt thereof, or a composition comprising a compound of Formulae (I-1), (I-2), (1-3) or (1-4), or a pharmaceutically acceptable salt thereof) to a subject exhibiting symptoms of a respiratory condition, results in reduction of the severity of inflammation-related pulmonary pain in a subject with a respiratory condition or retard or slow the progression of inflammation-related pulmonary pain in a subject with a respiratory condition.

In some embodiments, a subject with a respiratory condition is undergoing or has undergone treatment for an infection, fibrosis, a fibrotic episode, chronic obstructive pulmonary disease, Sarcoidosis (or pulmonary sarcoidosis) or asthma/asthma-related inflammation.

In some embodiments, a subject exhibits symptoms of and/or has been diagnosed with asthma. In some embodiments, a subject is or has undergone an asthmatic attack.

In some embodiments, a subject is undergoing or has undergone treatment for fibrosis or a fibrotic episode. In some embodiments, the fibrosis is cystic fibrosis.

In some embodiments, a respiratory condition is the result of and/or related to a disease or condition selected from the group consisting of cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, pulmonary sarcoidosis, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer's disease, Parkinson's disease, amyotrophic lateral sclerosis, progressive supranuclear palsy, Pick's disease, several polyglutamine neurological disorders such as Huntington, spinocerebellar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, COPD, dry-eye disease, or Sjogren's disease.

### Infections

The present disclosure contemplates, among other things, treatment of a subject who has an infection. The present disclosure contemplates, among other things, treatment of a subject who has a disease associated with an infection. In some embodiments, an infection is a viral infection or a bacterial infection. In some embodiments, an infection is a viral infection. In some embodiments, an infection is a bacterial infection.

In some embodiments, a viral infection is an infection of a virus selected from the group consisting of a coronavirus, an influenza virus, human rhinovirus, a human parainfluenza virus, human metapneumovirus and a hantavirus. In some embodiments, a virus is a coronavirus. In some embodiments, a coronavirus is selected from the group consisting of SARS-CoV, SARS-CoV-2, and MERS-CoV.

The present disclosure contemplates, among other things, treatment of a subject who has a disease associated with coronavirus. In some embodiments, a disease associated with a coronavirus is selected from the group consisting of coronavirus disease 2019 (COVID-19), severe acute respiratory syndrome (SARS) and Middle East respiratory syndrome (MERS). In some embodiments, a disease associated with a coronavirus is selected from the group consisting of COVID-19. In some embodiments, a coronavirus is selected from a group consisting of SARS-CoV-1, SARS-CoV-2, and 2012-nCoV. In some embodiments, a coronavirus is SARS-CoV-2.

In some embodiments, a bacterial infection is an infection of a bacteria selected from the group consisting of Streptococcus pneumoniae, Chlamydia pneumoniae, Staphylococcus aureus, Pseudomonas aeruginosa, and Haemophilus influenzae. In some embodiments, Staphylococcus aureus is methicillin-resistant Staphylococcus aureus.

### Enumerated Embodiments

The present disclosure includes the following embodiments numbered 1-109:
1. A compound of Formula (1-I):

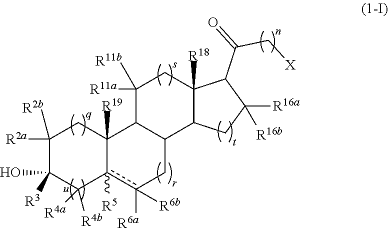

or a pharmaceutically acceptable salt thereof;
wherein:
q is independently 0, 1, 2, or 3;
r is independently 0, 1 or 2;
s is independently 0, 1 or 2;
t is independently 0, 1, 2 or 3;
n is independently 1 or 2;
u is independently 1 or 2;
X is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═OZ)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R^{A2}, —OS(═O)₂OR^{A1}, —S—S(═OZ)₂R^{A2}, —S—S(═O)₂OR^{A1}, —S(═OZ)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, or a nitrogen protecting group when attached to a nitrogen atom; and each instance of R^{A2} is independently substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R⁵ is hydrogen or methyl, or when [symbol] is a double bond, R⁵ and one of R^{6a} or R^{6b} is absent;
R¹⁹ is hydrogen or substituted or unsubstituted alkyl;
R¹⁸ is substituted or unsubstituted alkyl, substituted or unsubstituted alkenyl, or substituted or unsubstituted alkynyl;
R³ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each of R^{6a} and R^{6b} is independently hydrogen, halogen, cyano, hydroxyl, substituted or unsubstituted alkyl, substituted or unsubstituted alkenyl, or substituted or unsubstituted alkynyl; or R^{6a} and R^{6b} are joined to form an oxo (═O) group; and
each of R^{2a}, R^{2b}, R^{4a}, R^{4b}, R^{11a}, R^{11b}, R^{16a}, or R^{16b} is independently hydrogen, halogen, cyano, hydroxyl, substituted or unsubstituted alkyl, substituted or unsubstituted alkenyl, substituted or unsubstituted heterocyclyl, substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{2a} and R^{2b}, or R^{4a} and R^{4b}, or R^{1a} and R^{11b}, or R^{16a} and R^{16b} are joined to form an oxo (═O) group; provided that: q, s, r, u, and t are not simultaneously 1.
2. The compound of embodiment 1, wherein R^{2a} and R^{2b} is each independently hydrogen, halogen, cyano, hydroxyl, substituted or unsubstituted alkyl, substituted or unsubstituted alkenyl, substituted or unsubstituted heterocyclyl, substituted or unsubstituted alkynyl, —OR^{D1}, —OC(═O)R^{D1}, —NH₂, —N(R^{D1})₂, or —NR^{D1}C(═O)R^{D1}; wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
3. The compound of embodiment 1 or 2, wherein R^{2a} and R^{2b} is each independently hydrogen, halogen, cyano, hydroxyl, substituted or unsubstituted alkyl, —OR^{D1}, or —OC(═O)R^{D1}; wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
4. The compound of any one of embodiments 1-3, wherein R^{2a} and R^{2b} is each independently hydrogen, halogen, cyano, hydroxyl, substituted or unsubstituted alkyl, or —OR^{D1}; wherein each instance of R^{D1} is independently hydrogen, or substituted or unsubstituted alkyl.
5. The compound of any one of embodiments 1-4, wherein R^{2a} and R^{2b} are each independently hydrogen.
6. The compound of any one of embodiments 1-5, wherein R^{2a} and R^{2b} are both hydrogen.
7. The compound of any one of embodiments 1-6, wherein R^{4a} and R^{4b} is each independently hydrogen, halogen, cyano, hydroxyl, substituted or unsubstituted alkyl, substituted or unsubstituted alkenyl, substituted or unsubstituted heterocyclyl, substituted or unsubstituted alkynyl, —OR^{D1}, —OC(═O)R^{D1}, NH₂, —N(R^{D1})₂, or —NR^{D1}C(═O)R^{D1}; wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
8. The compound of any one of embodiments 1-7, wherein R^{4a} and R^{4b} is each independently hydrogen, halogen, cyano, hydroxyl, substituted or unsubstituted alkyl, —OR^{D1}, or —OC(═O)R^{D1}; wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
9. The compound of any one of embodiments 1-8, wherein R^{4a} and R^{4b} is each independently hydrogen, halogen, cyano, hydroxyl, substituted or unsubstituted alkyl, or -OR^{D1}; wherein each instance of R^{D1} is independently hydrogen, or substituted or unsubstituted alkyl.
10. The compound of any one of embodiments 1-9, wherein R^{4a} and R^{4b} are each independently hydrogen.
11. The compound of any one of embodiments 1-10, wherein R⁴ and R^{4b} are both hydrogen.
12. The compound of any one of embodiments 1-11, wherein R^{6a} and R^{6b} is each independently hydrogen, halogen, cyano, hydroxyl, substituted or unsubstituted alkyl, substituted or unsubstituted alkenyl, substituted or unsubstituted heterocyclyl, substituted or unsubstituted alkynyl, —OR^{D1}, —OC(═O)R^{D1}, —NH, —N(R^{D1})₂, or —NR^{D1}C(═O)R^{D1}; wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
13. The compound of any one of embodiments 1-12, wherein R^{6a} and R^{6b} is each independently hydrogen, halogen, cyano, hydroxyl, substituted or unsubstituted alkyl, —OR^{D1} or —OC(═O)R^{D1}, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
14. The compound of any one of embodiments 1-13, wherein R^{6a} and R^{6b} is each independently hydrogen, halogen, cyano, hydroxyl, substituted or unsubstituted alkyl, or —OR^{D1}; wherein each instance of R^{D1} is independently hydrogen, or substituted or unsubstituted alkyl.
15. The compound of any one of embodiments 1-14, wherein R^{6a} and R^{6b} are each independently hydrogen.
16. The compound of any one of embodiments 1-15, wherein R^{6a} and R^{6b} are both hydrogen.
17. The compound of any one of embodiments 1-16, wherein R^{11a} and R^{11b} is each independently hydrogen, halogen, cyano, hydroxyl, substituted or unsubstituted alkyl, substituted or unsubstituted alkenyl, substituted or unsubstituted heterocyclyl, substituted or unsubstituted alkynyl, —OR^{D1}, —OC(═O)R^{D1}, —NH₂, —N(R^{D1})₂, or —NR^{D1}C(═O)R^{D1}; wherein each instance of RD is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
18. The compound of any one of embodiments 1-17, wherein R^{11a} and R^{11b} is each independently hydrogen, halogen, cyano, hydroxyl, substituted or unsubstituted alkyl, —OR^{D1} or —OC(═O)R^{D1}; wherein each instance of R¹ is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
19. The compound of any one of embodiments 1-18, wherein R^{11a} and R^{11b} is each independently hydrogen, halogen, cyano, hydroxyl, substituted or unsubstituted alkyl, or —OR^{D1}; wherein each instance of R^{D1} is independently hydrogen, or substituted or unsubstituted alkyl.
20. The compound of any one of embodiments 1-19, wherein R^{11a} and R^{11b} are each independently hydrogen.
21. The compound of any one of embodiments 1-20, wherein R^{11a} and R^{11b} are both hydrogen.
22. The compound of any one of embodiments 1-21, wherein R^{16a} and R^{16b} is each independently hydrogen, halogen, cyano, hydroxyl, substituted or unsubstituted alkyl, substituted or unsubstituted alkenyl, substituted or unsubstituted heterocyclyl, substituted or unsubstituted alkynyl, —OR^{D1}, —OC(═O)R^{D1} —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
23. The compound of any one of embodiments 1-22, wherein R^{16a} and R^{16b} is each independently hydrogen, halogen, cyano, hydroxyl, substituted or unsubstituted alkyl, —OR^{D1} or —OC(═O)R^{D1}; wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
24. The compound of any one of embodiments 1-23, wherein R^{16a} and R^{16b} is each independently hydrogen, halogen, cyano, hydroxyl, substituted or unsubstituted alkyl, or —OR^{D1}; wherein each instance of R^{D1} is independently hydrogen, or substituted or unsubstituted alkyl.
25. The compound of any one of embodiments 1-24, wherein R^{16a} and R^{16b} are each independently hydrogen.
26. The compound of any one of embodiments 1-25, wherein R^{16a} and R^{16b} are both hydrogen.
27. The compound of any one of embodiments 1-26, wherein R⁵ is hydrogen in the cis position.
28. The compound of any one of embodiments 1-26, wherein R⁵ is hydrogen in the trans position.
29. The compound of any one of embodiments 1-26, wherein R⁵ is methyl in the cis position.
30. The compound of any one of embodiments 1-26, wherein R⁵ is methyl in the trans position.
31. The compound of any one of embodiments 1-30, wherein [symbol] is a single bond.
32. The compound of any one of embodiments 1-30, wherein [symbol] is a double bond.
33. The compound of any one of embodiments 1-32, wherein r is 1 and s is 1.
34. The compound of any one of embodiments 1-33, wherein t is 2.
35. The compound of any one of embodiments 1-34, wherein t is 3.
36. The compound of any one of embodiments 1-35, wherein q is 2.
37. The compound of any one of embodiments 1-36, wherein q is 0, 2, or 3; t is 0, 2, or 3, and u is 1.
38. The compound of any one of embodiments 1-35, wherein q is 2,t is 2, and u is 1.
39. The compound of any one of embodiments 1-38, wherein R³ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
40. The compound of any one of embodiments 1-39, wherein R³ is substituted or unsubstituted alkyl.
41. The compound of any one of embodiments 1-40, wherein R¹⁹ is substituted alkyl.
42. The compound of any one of embodiment 1-41, wherein R¹⁹ is unsubstituted alkyl.
43. The compound of any one of embodiment 1-40, wherein R¹⁹ is methyl, ethyl, or hydrogen.
44. The compound of any one of embodiments 1-40, wherein R¹⁹ is hydrogen.
45. The compound of any one of embodiments 1-40, wherein R¹⁹ is methyl.
46. The compound of any one of embodiments 1-45, wherein X is hydrogen, substituted or unsubstituted alkyl, substituted or unsubstituted alkenyl, substituted or unsubstituted aryl, substituted or unsubstituted heterocyclyl, or substituted or unsubstituted heteroaryl.
47. The compound of any one of embodiments 1-46, wherein X is hydrogen, substituted or unsubstituted heteroaryl, or substituted or unsubstituted alkyl.
48. The compound of any one of embodiments 1-47, wherein X is a substituted or unsubstituted heteroaryl.
49. The compound of any one of embodiments 1-48, wherein X is a substituted or unsubstituted 5-10 membered heteroaryl.
50. The compound of any one of embodiments 1-49, wherein R¹⁸ is unsubstituted alkyl.
51. The compound of any one of embodiments 1-49, wherein R¹⁸ is substituted alkyl.
52. The compound of any one of embodiments 1-51, wherein n is 1.
53. The compound of any one of embodiments 1-51, wherein n is 2.
54. The compound of embodiment 1, wherein the compound is of Formula (1-II-a), Formula (1-II-b), Formula (1-II-c), or Formula (1-II-d):

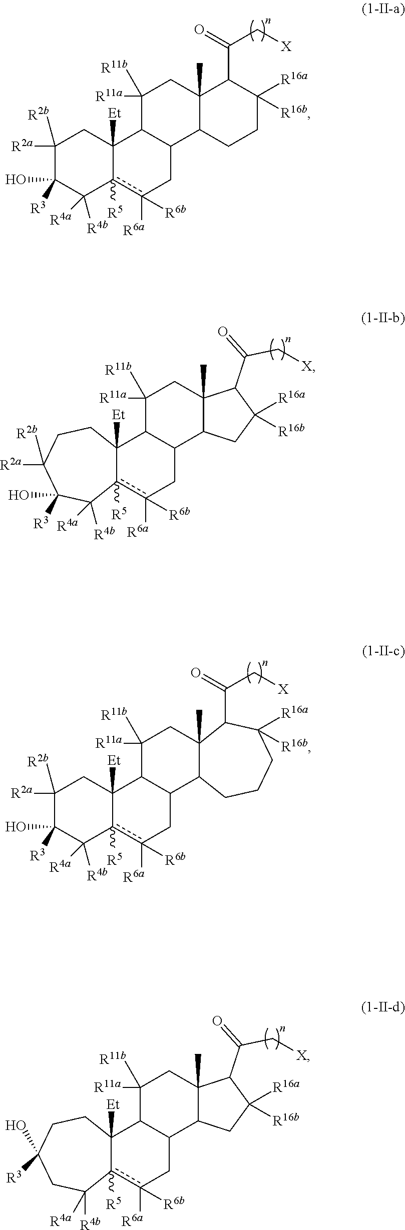

or a pharmaceutically acceptable salt thereof.
55. The compound of embodiment 1, wherein the compound is of Formula (1-III-a), Formula (1-III-b), Formula (1-III-c), or Formula (1-III-d).

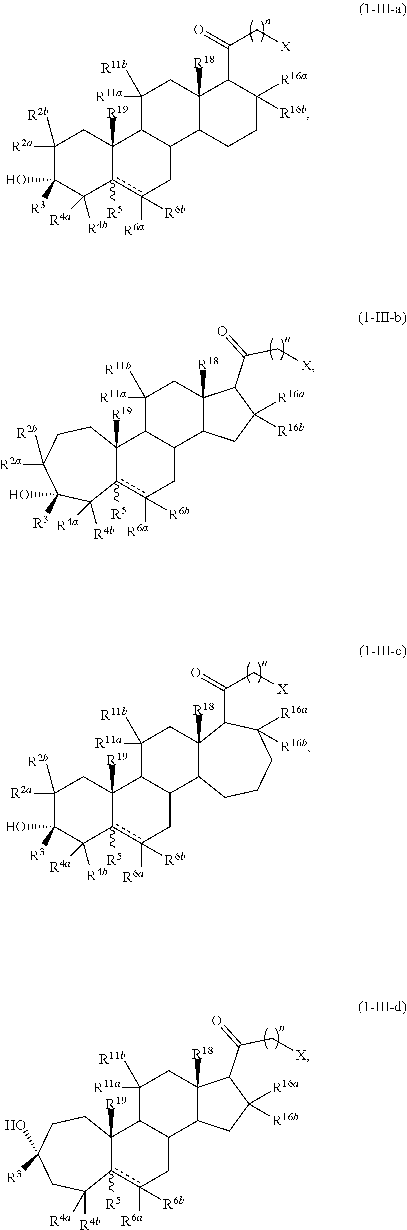

or a pharmaceutically acceptable salt thereof.
56. The compound of embodiment 55, wherein R¹⁸ is substituted or unsubstituted C₁₋₄ alkyl.
57. The compound of embodiment 55, wherein R¹⁸ is unsubstituted C₁₋₄ alkyl.
58. The compound of embodiment 55, wherein R¹⁸ is substituted C₁₋₄ alkyl.
59. The compound of embodiment 1, wherein the compound is of Formula (1-IV-a), Formula (1-IV-b), Formula (1-IV-c), or Formula (1-IV-d):

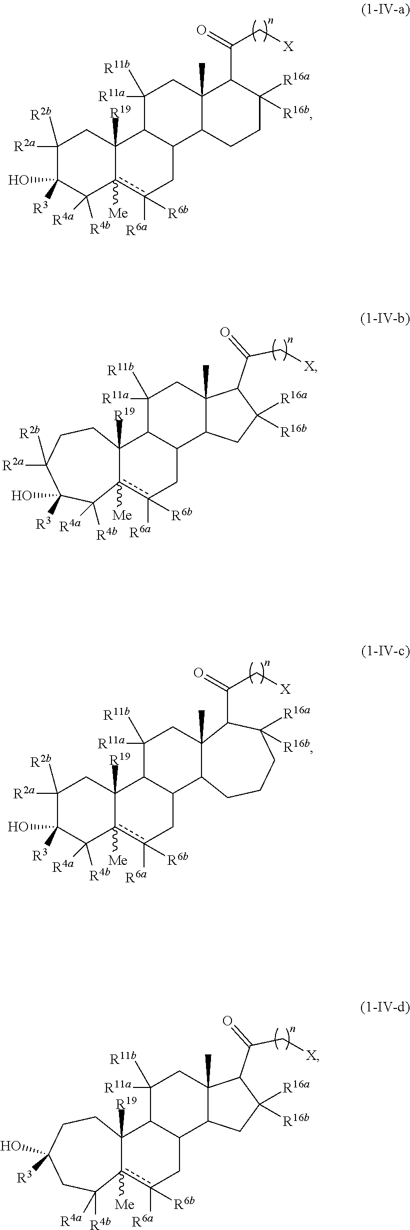

or a pharmaceutically acceptable salt thereof.
60. A compound of Formula (1-V-a) or (1-V-b):

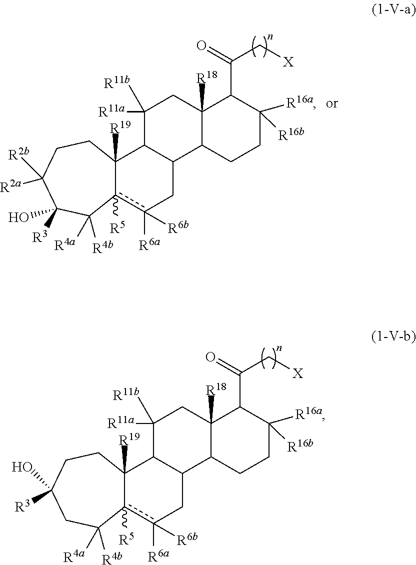

or a pharmaceutically acceptable salt thereof;
wherein:
   n is 1 or 2;
   X is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, NHC(═O)OR^{A1}, NHC(═O)SR^{A1}, NHC(═O)N(R^{A1})₂, OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}; wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom; and each instance of R^{A2} is independently substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl, or when [symbol] is a double bond, R⁵ is absent;
   R¹⁹ is hydrogen or substituted or unsubstituted alkyl;
   R¹⁸ is substituted or unsubstituted alkyl, substituted or unsubstituted alkenyl, or substituted or unsubstituted alkynyl;
   R³ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   each of R^{6a} and R^{6b} is independently hydrogen, halogen, cyano, hydroxyl, substituted or unsubstituted alkyl, substituted or unsubstituted alkenyl, or substituted or unsubstituted alkynyl; or R^{6a} and R^{6b} are joined to form an oxo (═O) group; and
   each of R^{2a}, R^{2b}, R^{4a}, R^{4b}, R^{11a}, R^{11b}, R^{16a}, or R^{16b} is independently hydrogen, halogen, cyano, hydroxyl, substituted or unsubstituted alkyl, substituted or unsubstituted alkenyl, substituted or unsubstituted heterocyclyl, substituted or unsubstituted alkynyl, —OR^{D1}, —OC(═O)R^{D1}, —NH₂, —N(R^{D1})₂, or —NR^{D1}C(═O)R^{D1}; wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, or a nitrogen protecting group when attached to a nitrogen atom; or two R^{D1} groups are joined to form an substituted or unsubstituted heterocyclic ring; or any one of R^{2a} and R^{2b}, or R^{4a} and R^{4b}, or R^{11a} and R^{11b}, or R^{16a} and R^{16b} are joined to form an oxo (═O) group.
61. The compound of embodiment 60, wherein n is 1.
62. The compound of embodiment 60, wherein n is 2.
63. The compound of any one of embodiments 60-62, wherein X is hydrogen, halogen, substituted or unsubstituted alkyl, substituted or unsubstituted heterocyclyl, substituted or unsubstituted carbocyclyl, substituted or unsubstituted aryl, substituted or unsubstituted heteroaryl, or —OR^{A1}; wherein R^{A1} is independently hydrogen, substituted or unsubstituted alkyl, substituted or unsubstituted carbocyclyl, substituted or unsubstituted aryl, or substituted or unsubstituted heteroaryl.
64. The compound of any one of embodiments 60-63, wherein X is hydrogen, or substituted or unsubstituted heteroaryl.
65. The compound of any one of embodiments 60-64, wherein X is a substituted or unsubstituted N-linked heteroaryl.
66. The compound of any one of embodiments 60-65, wherein the N-linked heteroaryl is a 5-6 membered N-linked heteroaryl.
67. The compound of any one of embodiments 60-63, wherein X is:

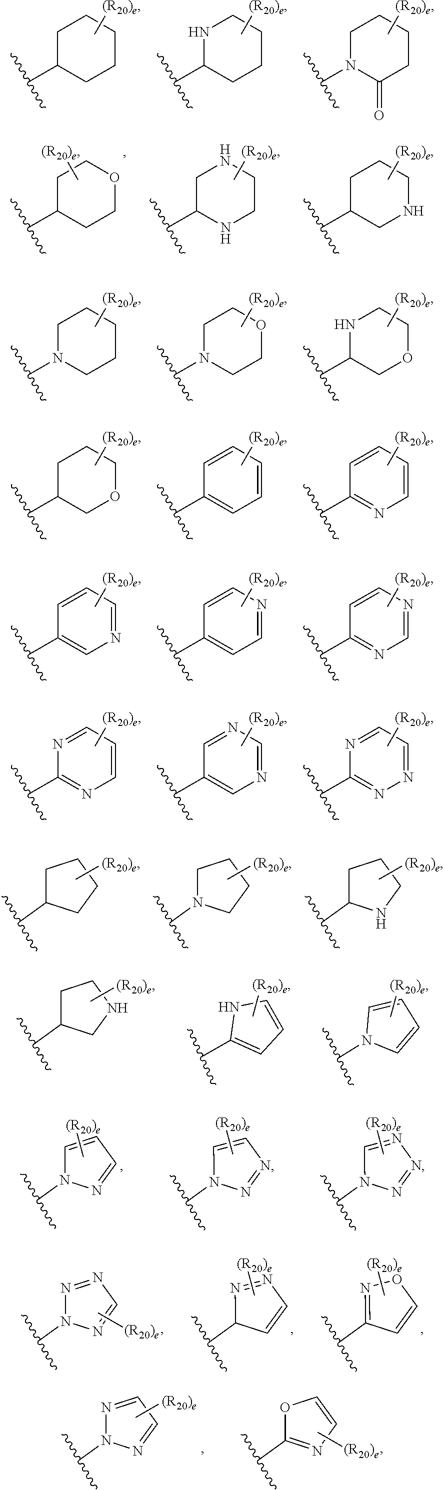

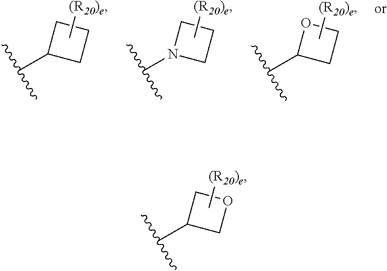

wherein each instance of R₂₀ is independently halogen, —NO₂, —CN, —OR^{GA}, —N(R^{GA})₂, —C(═O)R^{GA}, —C(═O)OR^{GA}, —OC(═O)R^{GA}, —OC(═O)OR^{GA}, —C(═O)N(R^{GA})₂, —N(R^{GA})C(═O)R^{GA}, —OC(═O)N(R^{GA})₂, —N(R^{GA})C(═O)OR^{GA}, —S(═O)₂R^{GA}, —S(═O)₂OR^{GA}, —OS(═O)₂R^{GA}, —S(═O)₂N(R^{GA})₂, or —N(R^{GA})S(═O)₂R^{GA}; substituted or unsubstituted C₁₋₆ alkyl, substituted or unsubstituted C₂₋₆ alkenyl, substituted or unsubstituted C₂₋₆ alkynyl, substituted or unsubstituted C₃₋₄ carbocylyl, or substituted or unsubstituted 3- to 4-membered heterocylyl;
wherein each instance of R^{GA} is independently hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3-6 membered heterocylyl, substituted or unsubstituted aryl, substituted or unsubstituted heteroaryl, an oxygen protecting group when attached to oxygen, or a nitrogen protecting group when attached to nitrogen; or two R^{GA} groups are taken with the intervening atoms to form a substituted or unsubstituted carbocyclic or substituted or unsubstituted heterocyclic ring;
e is 0, 1, 2, 3, 4, or 5.
68. The compound of any one of embodiments 60-63, wherein X is:

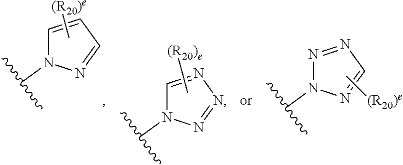

wherein each instance of R₂₀ is, independently, halogen, —NO₂, —CN, —OR^{GA}, —N(R^{GA})₂, —C(═O)R^{GA}, —C(═O)OR^{GA}, —C(═O)N(R^{GA})₂, —N(R^{GA})C(═O)R^{GA}, —OC(═O)N(R^{GA})₂, substituted or unsubstituted C₁₋₆ alkyl, substituted or unsubstituted 3-4 membered carbocylyl, substituted or unsubstituted 3-4 membered heterocyclyl;
wherein each instance of R^{GA} is independently hydrogen, substituted or unsubstituted C₁₋₆ alkyl, substituted or unsubstituted aryl, substituted or unsubstituted heteroaryl, an oxygen protecting group when attached to oxygen, a nitrogen protecting group when attached to nitrogen, or two R^{GA} groups are taken with the intervening atoms to form a substituted or unsubstituted carbocyclic or heterocyclic ring; and
e is 0, 1, 2, or 3.
69. The compound of embodiment 68, wherein R₂₀ is CN.
70. The compound of embodiment 68, wherein R₂₀ is unsubstituted alkyl.
71. The compound of embodiment 70, wherein R₂₀ is unsubstituted C₁₋₆alkyl.
72. The compound of embodiment 71, wherein R₂₀ is methyl.
73. The compound of any one of embodiments 60-72, wherein R⁵ is hydrogen in the cis position.
74. The compound of any one of embodiments 60-72, wherein R⁵ is hydrogen in the trans position.
75. The compound of any one of embodiments 60-74, wherein [symbol] is a single bond.
76. The compound of any one of embodiments 60-74, wherein [symbol] is a double bond.
77. The compound of any one of embodiments 60-76 wherein R³ is substituted or unsubstituted alkyl.
78. The compound of any one of embodiments 77, wherein R³ is unsubstituted alkyl.
79. The compound of embodiment 78, wherein R³ is R³ is methyl, ethyl, —CH₂OCH₃, or —CH₂OCH₂CH₃.
80. The compound of embodiment 77, wherein alkyl is optionally substituted with halo, or OR^{D1}
81. The compound of embodiment 80, wherein R^{D1} is hydrogen or substituted or unsubstituted alkyl.
82. The compound of embodiment 81 wherein R³ is —CH₂OCH₃.
83. The compound of any one of embodiments 60-82, wherein R¹⁹ is substituted alkyl.
84. The compound of any one of embodiments 60-82, wherein R¹⁹ is unsubstituted alkyl.
85. The compound of any one of embodiment 60-82, wherein R¹⁹ is methyl, ethyl or hydrogen.
86. The compound of any one of embodiments 60-85, wherein R^{1a} is unsubstituted alkyl.
87. The compound of embodiment 86, wherein R¹⁸ is methyl.
88. The compound of any one of embodiments 60-85, wherein R¹⁸ is substituted alkyl.
89. The compound of any one of embodiments 60-88, wherein each of R^{2a}, R^{2b}, R^{4a}, R^{4b}, R^{6a}, R^{6b}, R^{11a}, R^{11b}, R^{16a}, or R^{16b} is independently hydrogen, substituted or unsubstituted alkyl, substituted or unsubstituted alkenyl, —OH, or —OR^{D1}; or any one of R^{2a} and R^{2b}, or R^{4a} and R^{4b}, or R^{11a} and R^{1b}, or R^{16a} and R^{16b} are joined to form an oxo (═O) group; wherein each alkyl is optionally substituted with a substitutent selected from halo, —OH, or —OR^{D1}; and wherein each R^{D1} is independently hydrogen, haloalkyl, or unsubstituted alkyl.
90. The compound of any one of embodiments 60-89, wherein R^{2a}, R^{2b}, R^{4a}, R^{4b}, R^{6a}a, R^{6b}, R^{11a}, R^{11b}, R^{16a}a, or R^{16b} are hydrogen.
91. The compound of any one of embodiments 60-90, wherein the compound is selected from the group consisting of:

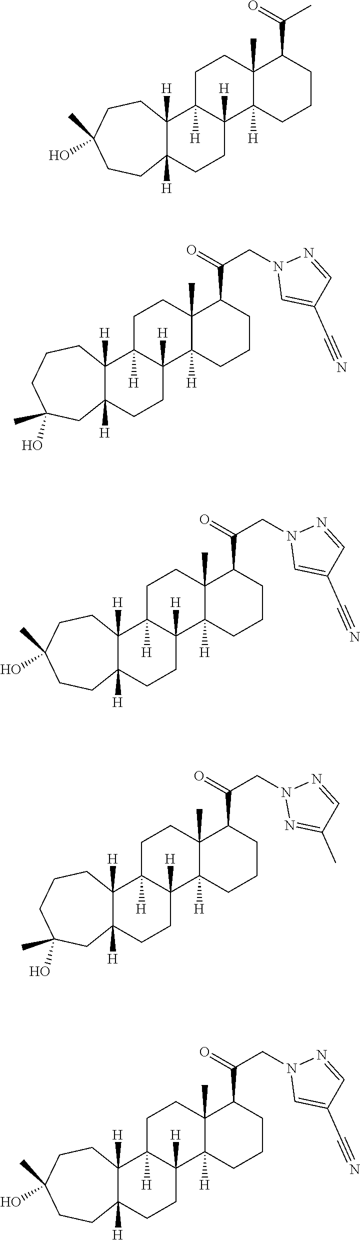

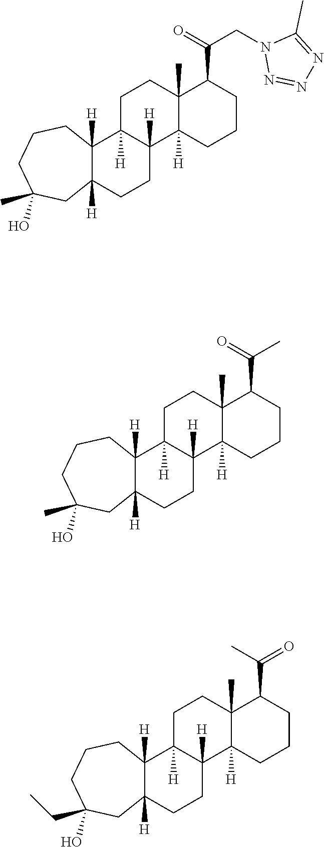

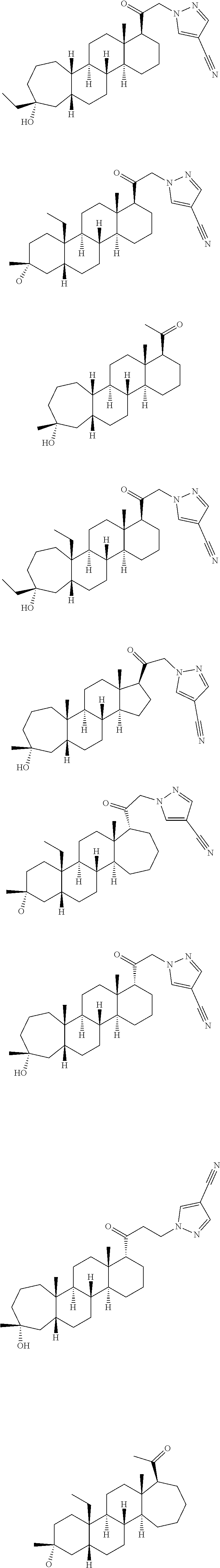

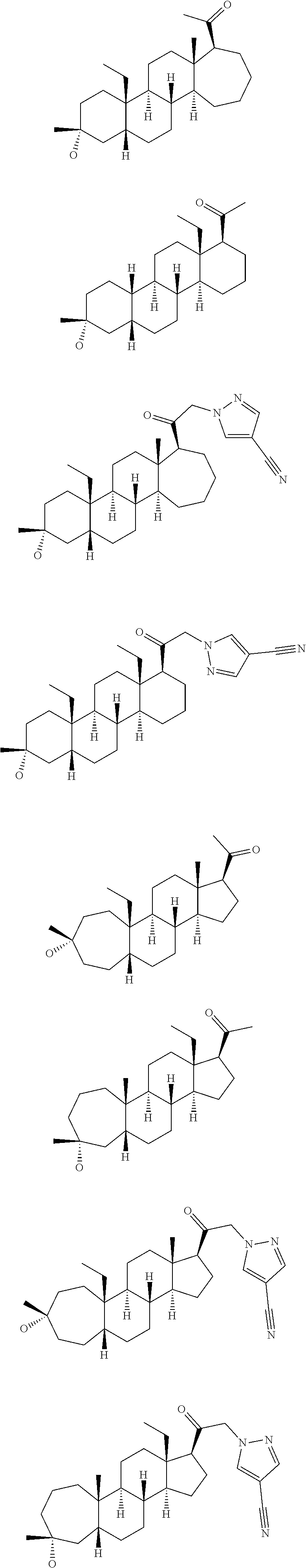

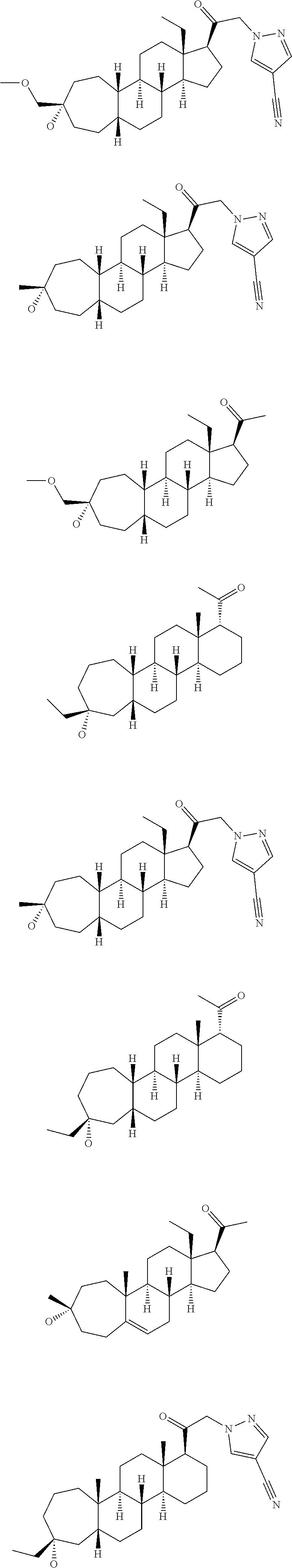

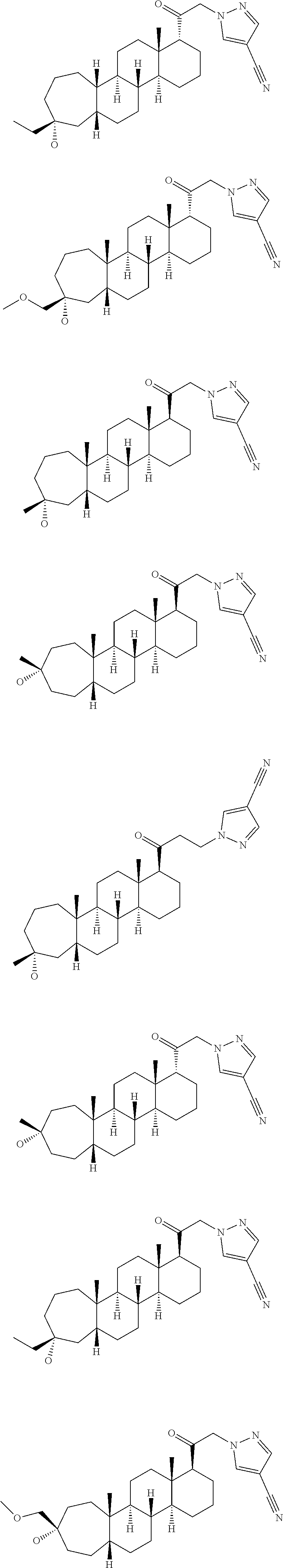

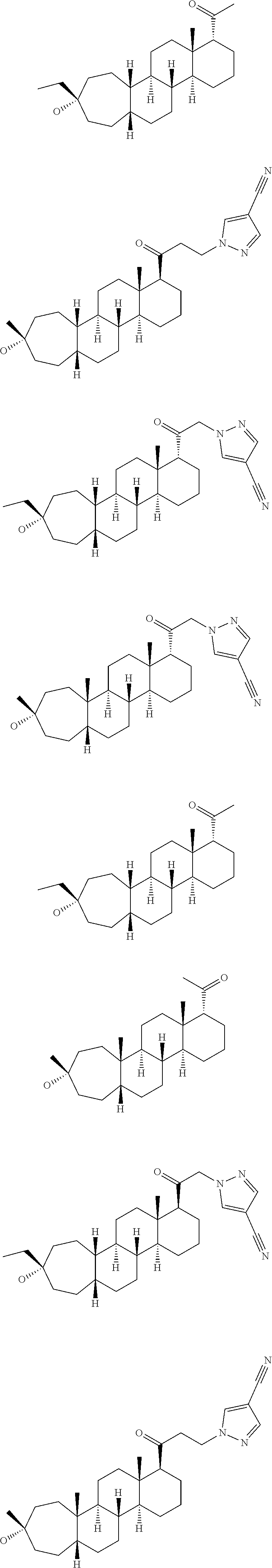

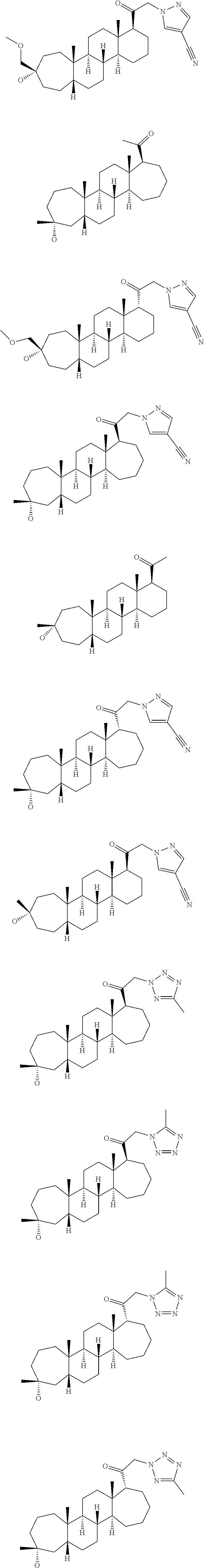

92. The compound of embodiment 1, wherein the compound is of Formula (1-VI-a) or Formula (1-VIb):

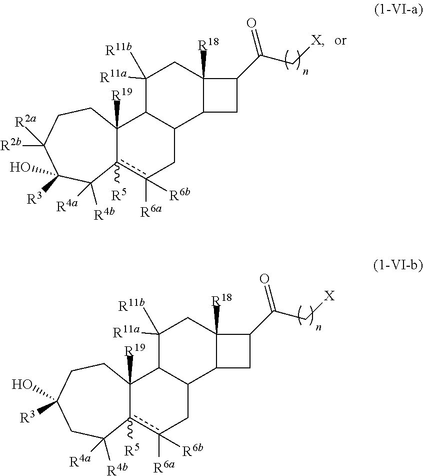

or a pharmaceutically acceptable salt thereof.
93. The compound of any one of embodiments 1-53, wherein the compound is of Formula (1-VII-a) or Formula (1-VII-b):

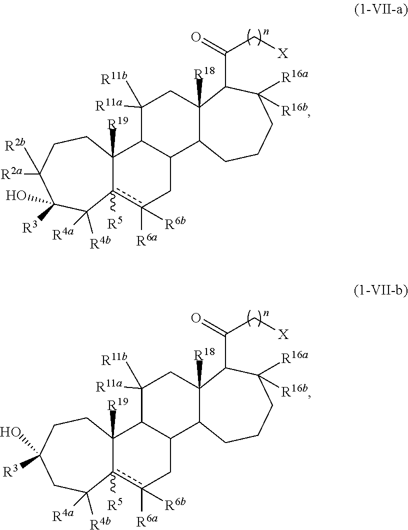

or a pharmaceutically acceptable salt thereof.
94. The compound of embodiment 1, wherein the compound is of Formula (1-VIII-a) or Formula (1-VIII-b):

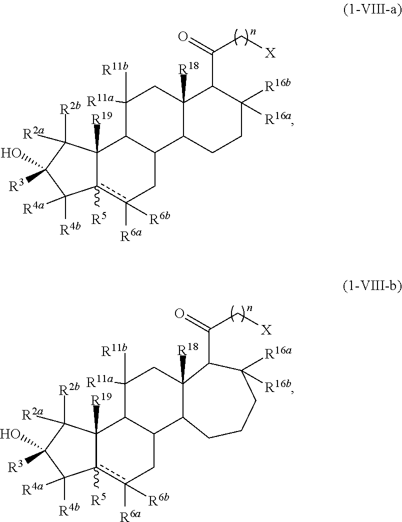

or a pharmaceutically acceptable salt thereof.
95. The compound of embodiment 1, wherein the compound is of Formula (1-IX-a), Formula (1-TX-b), Formula (1-TX-c), or Formula (1-TX-d):

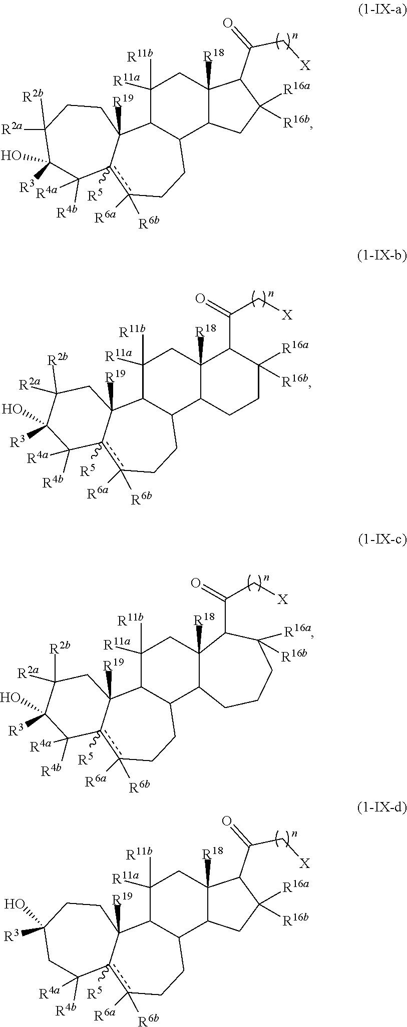

or a pharmaceutically acceptable salt thereof.
96. The compound of embodiment 1, wherein the compound is of Formula (1-X-a), Formula (1-X-b), Formula (1-X-c), or Formula (1-X-d):

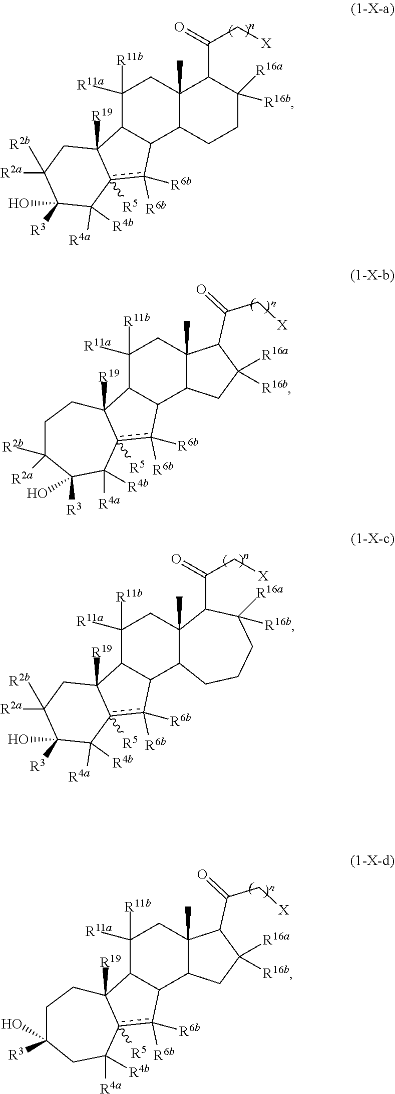

or a pharmaceutically acceptable salt thereof.
97. The compound of embodiment 1, wherein the compound is of Formula (1-XI-a), Formula (1-XI-b), or Formula (1-XI-c):

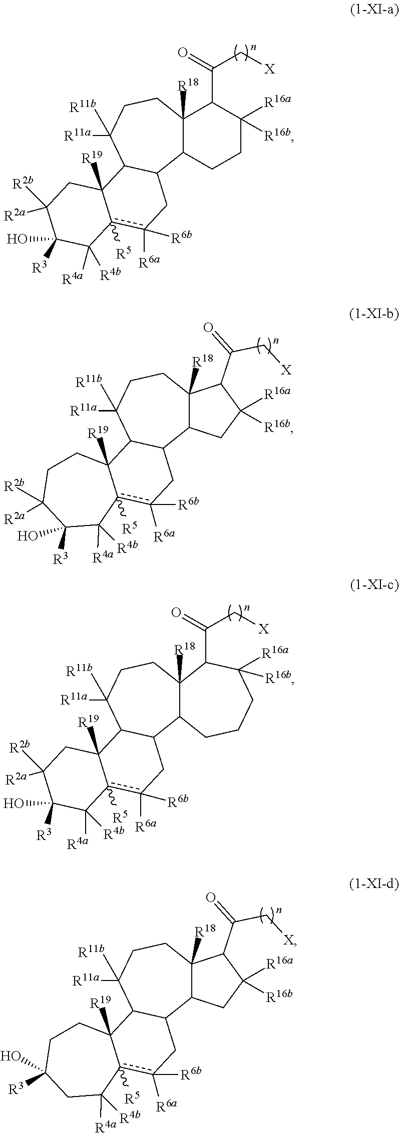

or a pharmaceutically acceptable salt thereof.
98. The compound of embodiment 1, wherein the compound is of Formula (1-XII-a), or Formula (1-XII-b):

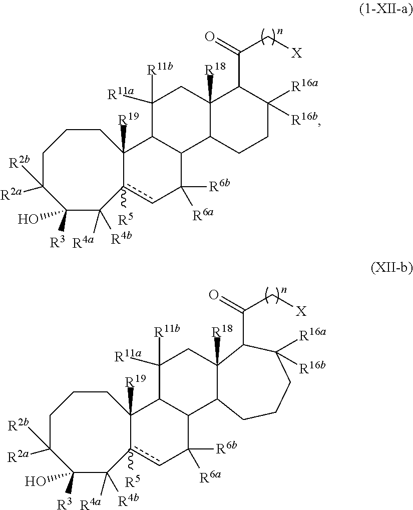

or a pharmaceutically acceptable salt thereof.
99. The compound of any one of embodiments 60-90, wherein the compound is a compound of Formula (1-XIII-a) or Formula (1-XIII-b):

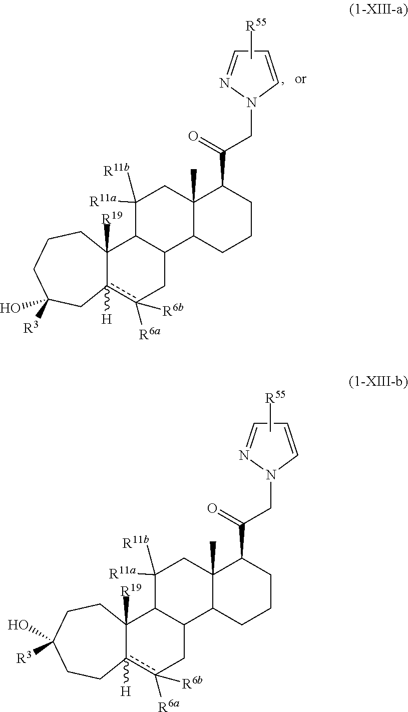

or a pharmaceutically acceptable salt thereof;
wherein R⁵⁵ is hydrogen, halogen, cyano, or substituted or unsubstituted alkyl.
100. The compound of any one of embodiments 60-90, wherein the compound is a compound of Formula (1-XIV-a) or Formula (1-XIV-b):

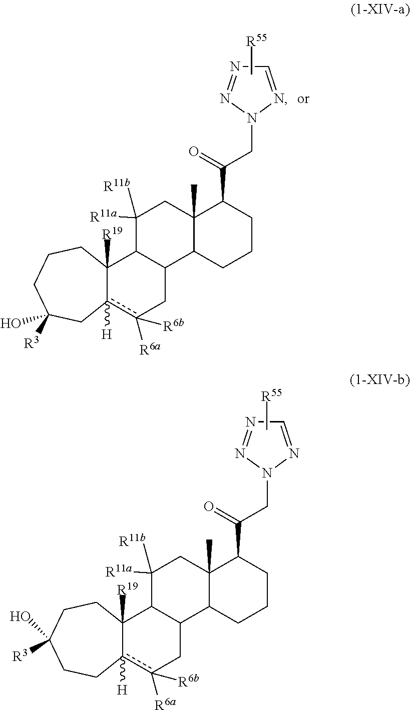

or a pharmaceutically acceptable salt thereof;
wherein R⁵ is hydrogen, halogen, cyano, or substituted or unsubstituted alkyl.
101. A pharmaceutical composition comprising a compound of any one of embodiments 1-100 or a pharmaceutically acceptable salt thereof and a pharmaceutically acceptable excipient.
102. A method of modulating a GABA_{A} receptor in a subject in need thereof, comprising administering to the subject a therapeutically effective amount of a compound of any one of embodiments 1-100 or a pharmaceutically acceptable salt thereof, or a pharmaceutical composition of embodiment 101.
103. A method of treating a CNS-related disorder in a subject in need thereof, comprising administering to the subject an effective amount of a compound of any one of embodiments 1-100 or a pharmaceutically acceptable salt thereof, or a pharmaceutical composition of embodiment 101.
104. The method of embodiment 103, wherein the CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, or status epilepticus.
105. The method of embodiment 104, wherein the CNS-related disorder is a mood disorder.
106. The method of embodiment 105, wherein the mood disorder is depression.
107. The method of embodiment 106, wherein the depression is postpartum depression.
108. The method of embodiment 104, wherein the CNS-related disorder is major depressive disorder.
108. The method of embodiment 108, wherein the major depressive disorder is moderate major depressive disorder.
109. The method of embodiment 104, wherein the major depressive disorder is severe major depressive disorder.

The present disclosure includes the following embodiments numbered 1a-145a:
1a. A compound of Formula 2-1:

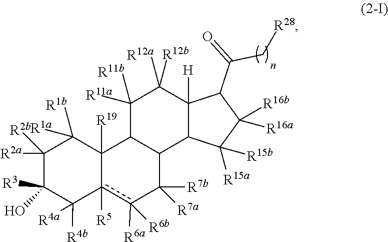

or a pharmaceutically acceptable salt thereof;
wherein:
   [symbol] represents a single or double bond, provided if a double bond is present, then R⁵ and one of R^{6a} or R^{6b} are absent;
   R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or substituted or unsubstituted methyl, or when [symbol] is a double bond, R⁵ is absent;
   each of R^{6a} and R^{6b} is independently hydrogen, halogen, cyano, hydroxyl, substituted or unsubstituted alkyl, substituted or unsubstituted alkenyl, or substituted or unsubstituted alkynyl, or R^{6a} and R^{6b} are joined to form an oxo (═O) group;
   each of R^{1a}, R^{1b}, R^{2a}, R^{2b}, R^{4a}, R^{4b}, R^{7a}, R^{7b} R^{11a}, R^{1b}, R^{12a}, R^{12b}, R¹⁵, and R^{15b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1} wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{1b}, R^{2a} and R^{12b}, and R^{15a} and R^{15b} are joined to form an oxo (═O) group;
   each of R^{16a} and R^{16b} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —N(R^{A1}) —CN(R^{A1})₂, —C(O)R^{A1}, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R¹⁹ is hydrogen, substituted or unsubstituted alkyl, substituted or unsubstituted alkenyl, or substituted or unsubstituted alkynyl;
   R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂ —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   n is 1, 2, or 3; and
   with the proviso that the compound is not:

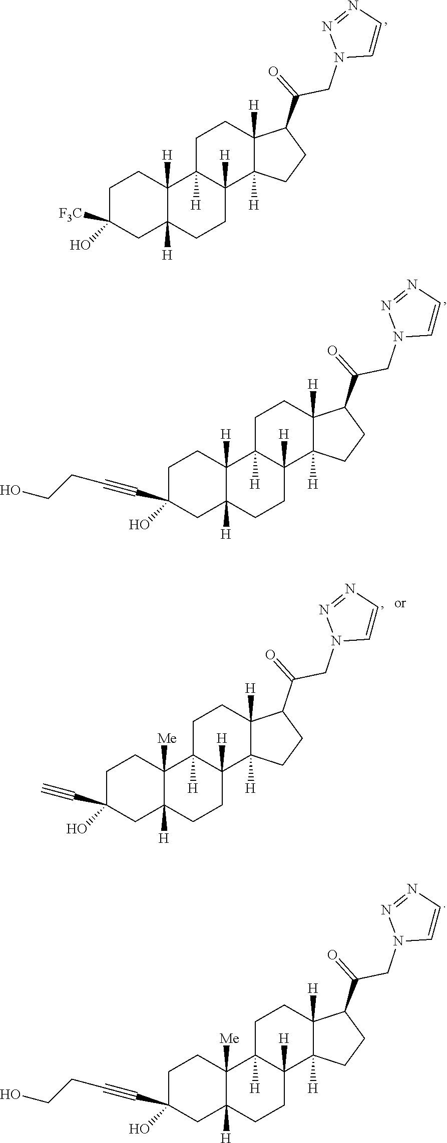

2a. A method of treating a CNS-related disorder in a subject in need thereof, comprising administering to the subject a compound of Formula 2-I:

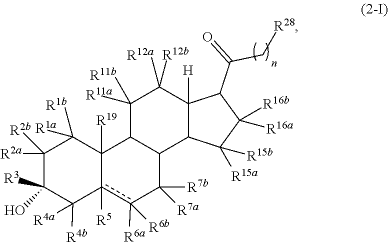

or a pharmaceutically acceptable salt thereof;
wherein:
   [symbol] represent a single or double bond, provided if a double bond is present, then R⁵ and one of R^{6a} or R^{6b} are absent;
   R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or substituted or unsubstituted methyl, or when [symbol] is a double bond, R⁵ is absent;
   each of R^{6a} and R^{6b} is independently hydrogen, halogen, cyano, hydroxyl, substituted or unsubstituted alkyl, substituted or unsubstituted alkenyl, or substituted or unsubstituted alkynyl, or R^{6a} and R^{6b} are joined to form an oxo (═O) group;
   each of R^{1a}, R^{1b}, R^{2a}, R^{2b}, R^{4a}, R^{4b}, R^{7a}, R^{7b}, R^{1a}, R^{1b}, R^{12a}, R^{12b}, R^{5a}, and R^{15b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{11b}, R^{12a} and R^{12b}, and R^{15a} and R^{15b} are joined to form an oxo (═O) group;
   each of R^{16a} and R^{16b} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —N(R^{A1}) —CN(R^{A1})₂, —C(O)R^{A1}, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, NHC(═O)N(R^{A1})₂, —OS(═O)₂R^{A2}, —OS(═O)₂OR^{A1}, —S—S(═O)₂R^{A2}, —S—S(═O)₂OR^{A7}, —S(═O)R^{A2}, —SO₂R^{A2}, or —S(═O)₂OR^{A7}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R¹⁹ is hydrogen, substituted or unsubstituted alkyl, substituted or unsubstituted alkenyl, or substituted or unsubstituted alkynyl;
   R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1} —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(RAJ)₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring; and
   n is 1, 2, or 3.
3a. The compound of embodiment 1a or 2a, wherein the compound is a compound of Formula 2-Ia or Formula 2-Ib:

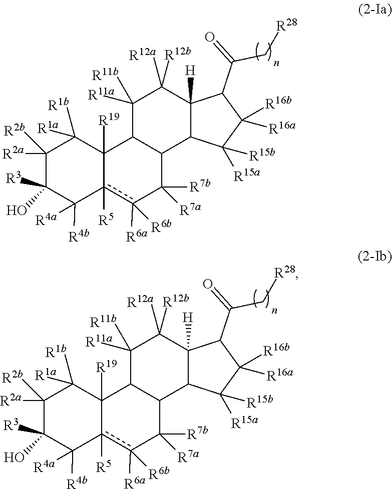

or a pharmaceutically acceptable salt thereof.
4a. The compound of any one of embodiments 1a-3a, wherein the compound is a compound of Formula 2-Iaa or Formula 2-Iab:

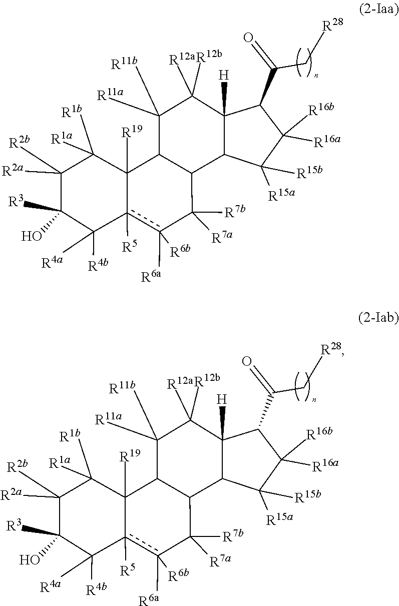

or a pharmaceutically acceptable salt thereof.
5a. A compound of Formula 2-II:

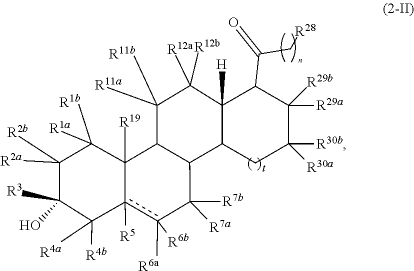

or a pharmaceutically acceptable salt thereof;
wherein:
   t is 1 or 2;
   [symbol] represents a single or double bond, provided if a double bond is present, then R⁵ and one of R^{6a} or R^{6b} are absent;
   R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or substituted or unsubstituted methyl, or when [symbol] is a double bond, R⁵ is absent;
   each of R^{6a} and R^{6b} is independently hydrogen, halogen, cyano, hydroxyl, substituted or unsubstituted alkyl, substituted or unsubstituted alkenyl, or substituted or unsubstituted alkynyl, or R^{6a} and R^{6b} are joined to form an oxo (═O) group;
   each of R^{1a}, R^{1b}, R^{2a}, R^{2b}, R^{4a}, R^{4b}, R^{7a}, R^{7b}, R^{1a}, R^{1b}, R^{12a}, R^{12b}, R^{30a}, and R^{30b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{11b}, R^{12a} and R^{12b}, and R^{30a} and R^{30b} are joined to form an oxo (═O) group;
   each of R^{29a} and R^{29b} is each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂—N(R^{A1}), —CN(R^{A1})₂, —C(O)R^{A1}, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R^{A2}, OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R¹⁹ is hydrogen, substituted or unsubstituted alkyl, substituted or unsubstituted alkenyl, or substituted or unsubstituted alkynyl;
   R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring; and
   n is 1, 2, or 3.
6a. The compound of embodiment 5a, wherein the compound is a compound of Formula 2-IIa

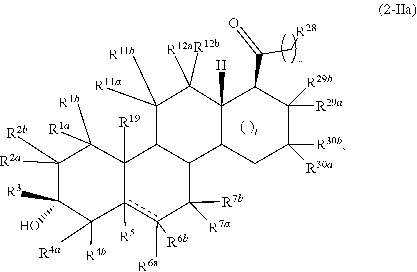

or a pharmaceutically acceptable salt thereof.
7a. A compound of Formula 2-III:

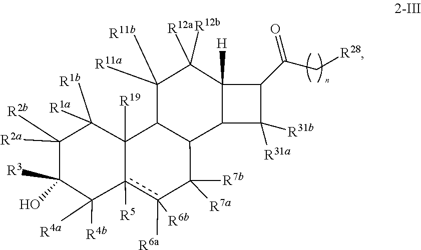

or a pharmaceutically acceptable salt thereof;
wherein:
   [symbol] represents a single or double bond, provided if a double bond is present, then R⁵ and one of R^{6a} or R^{6b} are absent;
   R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or substituted or unsubstituted methyl, or when [symbol] is a double bond, R⁵ is absent; each of R^{6a} and R^{6b} is independently hydrogen, halogen, cyano, hydroxyl, substituted or unsubstituted alkyl, substituted or unsubstituted alkenyl, or substituted or unsubstituted alkynyl, or R^{6a} and R^{6b} are joined to form an oxo (═O) group;
   each of R^{1a}, R^{1b}, R^{2a}, R^{2b}, R^{4a}, R^{4b}, R^{7a}, R^{7b}, R^{1a}, R^{1b}, R^{12a}, and R^{12b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, Rita and R^{1b}, R^{11a} and R^{12b} are joined to form an oxo (═O) group;
   each of R^{3a} and R^{31b} is each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —N(R^{A1}), —CN(R^{A1})₂, —C(O)R^{A1}, —OC(═O)R^{A1}, —OC(═O)OR^{A1}, —OC(═O)SR^{A1}, OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R², —OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R¹⁹ is hydrogen, substituted or unsubstituted alkyl, substituted or unsubstituted alkenyl, or substituted or unsubstituted alkynyl;
   R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring; and
   n is 1, 2, or 3.
8a. The compound of embodiment 7a, wherein the compound is a compound of Formula 2-IIIa:

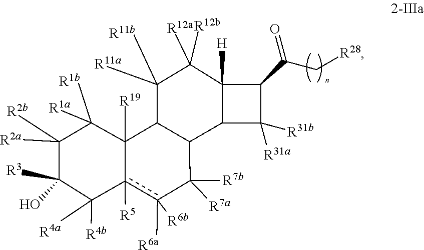

or a pharmaceutically acceptable salt thereof.
9a. A compound of Formula 2-IVa or Formula 2-IVb:

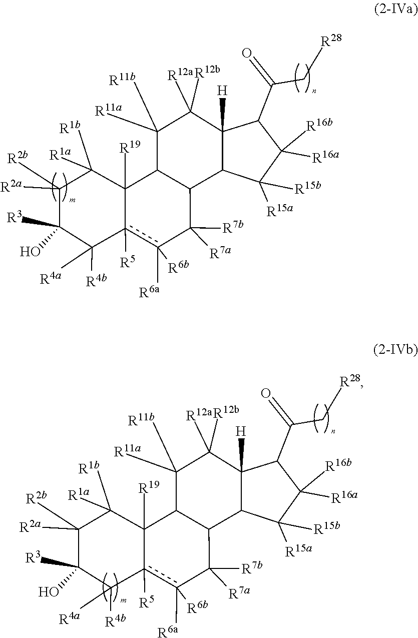

or a pharmaceutically acceptable salt thereof;
wherein:
   [symbol] represents a single or double bond, provided if a double bond is present, then R⁵ and one of R^{6a} or R^{6b} are absent;
   R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or substituted or unsubstituted methyl, or when [symbol] is a double bond, R⁵ is absent;
   each of R^{6a} and R^{6b} is independently hydrogen, halogen, cyano, hydroxyl, substituted or unsubstituted alkyl, substituted or unsubstituted alkenyl, or substituted or unsubstituted alkynyl, or R^{6a} and R^{6b} are joined to form an oxo (═O) group;
   each of R^{1a}, R^{1b}, R^{2a}, R^{2b}, R^{4a}, R^{4b}, R^{7a}, R^{7b}, R^{11a}, Rub, R^{12a}, R^{12b}, R^{15a}, and R^{15b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{DI} groups are joined to form an substituted or unsubstituted heterocyclic ring; or any one of R^{1a} and R^{1b}, R^{2a} and R^{2b}, R^{4a} and R^{4b}, R^{11a} and R^{1b}, R^{2a} and R^{12b}, and R^{5a} and R^{15b} are joined to form an oxo (═O) group;
   each of R^{16a} and R^{16b} is each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1} —N(R^{A1})₂, —N(R^{A1}), —CN(R^{A1})₂, —C(O)R^{A1}, —OC(═O)R^{A1}, —OC(═O)OR^{A1}, —OC(═O)SR^{A1}, OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R¹⁹ is hydrogen, substituted or unsubstituted alkyl, substituted or unsubstituted alkenyl, or substituted or unsubstituted alkynyl;
   R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   n is 1, 2, or 3; and
   m is 2 or 3.
10a. The compound of embodiment 9a, wherein the compound is a compound of Formula 2-IVaa or Formula 2-IVba:

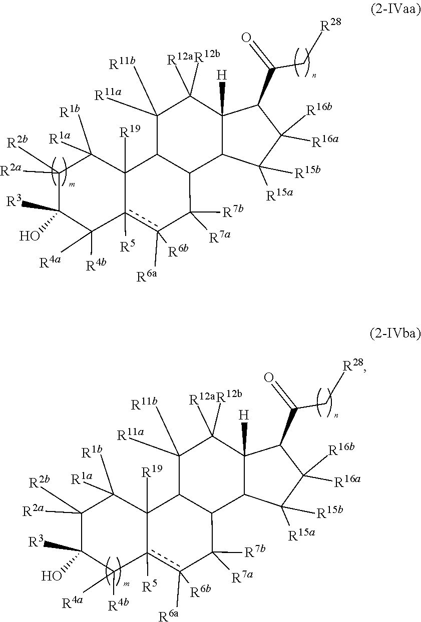

or a pharmaceutically acceptable salt thereof.
11a. A compound of Formula 2-V:

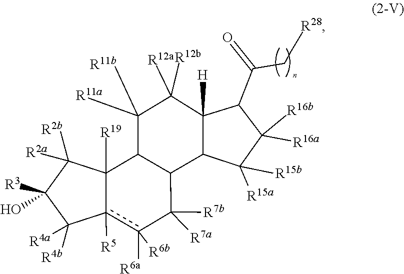

or a pharmaceutically acceptable salt thereof;
wherein:
   [symbol] represents a single or double bond, provided if a double bond is present, then R⁵ and one of R^{6a} or R^{6b} are absent;
   R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or substituted or unsubstituted methyl, or when [symbol] is a double bond, R⁵ is absent;
   each of R^{6a} and R^{6b} is independently hydrogen, halogen, cyano, hydroxyl, substituted or unsubstituted alkyl, substituted or unsubstituted alkenyl, or substituted or unsubstituted alkynyl, or R^{6a} and R^{6b} are joined to form an oxo (═O) group;
   each of R^{2a}, R^{2b}, R^{4a}, R^{4b}, R^{7a}, R^{7b}, R^{11a}, R^{11b}, R^{12a}, R^{12b}, R^{15a}, and R^{15b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1} wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{1b}, R^{2a} and R^{12b}, and R^{15a} and R^{15b} are joined to form an oxo (═O) group;
   each of R^{16a} and R^{16b} is each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —N(R^{A1}), —CN(R^{A1})₂, —C(O)R^{A1}, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R², —OS(═O)₂OR^{A1}, —S—S(═O)₂R^{A2}, —S—S(═O)₂OR^{A1}, —S(═O)R^{A2}, SO₂R^{A2}, or —S(═O)₂OR^{A1} wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R¹⁹ is hydrogen, substituted or unsubstituted alkyl, substituted or unsubstituted alkenyl, or substituted or unsubstituted alkynyl;
   R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂ —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring; and
   n is 1, 2, or 3.
12a. The compound of embodiment 11a, wherein the compound is a compound of Formula 2-Va:

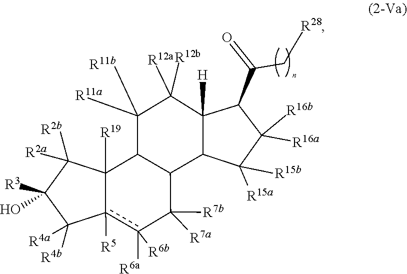

or a pharmaceutically acceptable salt thereof.
13a. A compound of Formula 2-VI:

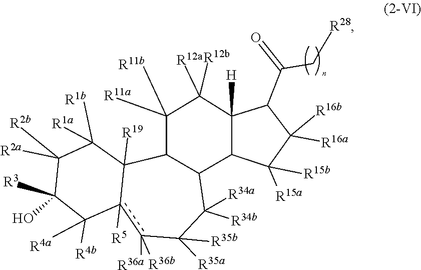

or a pharmaceutically acceptable salt thereof;
wherein:
   [symbol] represents a single or double bond, provided if a double bond is present, then R⁵ and one of R^{36a} or R^{36b} are absent;
   R³ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or substituted or unsubstituted methyl, or when [symbol] is a double bond, R⁵ is absent; each of R^{36a} and R^{36b} is independently hydrogen, halogen, cyano, hydroxyl, substituted or unsubstituted alkyl, substituted or unsubstituted alkenyl, or substituted or unsubstituted alkynyl, or R^{36a} and R^{36b} are joined to form an oxo (═O) group;
   each of R^{1a}, R^{1b}, R^{2a}, R^{2b}, R^{4a}, R^{4b}, R^{11a}, R^{11b}, R^{12a}, R^{12b}, R⁵, R^{15b}, R^{34a}, R^{34b}, R^{35a} and R^{35b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{11b}, R^{12a} and R^{12b}, and R^{15a} and R^{15b} are joined to form an oxo (═O) group;
   each of R^{16a} and R^{16b} is each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —N(R^{A1}), —CN(R^{A1})₂, —C(O)R^{A1}, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R¹⁹ is hydrogen, substituted or unsubstituted alkyl, substituted or unsubstituted alkenyl, or substituted or unsubstituted alkynyl;
   R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring; and
   n is 1, 2, or 3.
14a. The compound of embodiment 13a, wherein the compound is a compound of Formula 2-VIa:

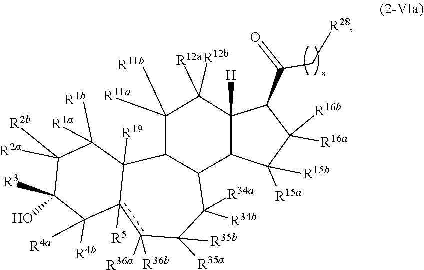

or a pharmaceutically acceptable salt thereof.
15a. A compound of Formula VII:

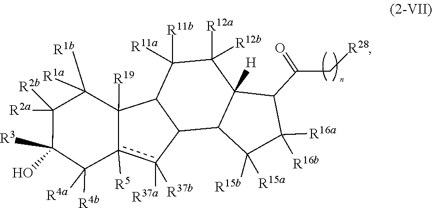

or a pharmaceutically acceptable salt thereof;
wherein:
   [symbol] represents a single or double bond, provided if a double bond is present, then R⁵ and one of R^{36a} or R^{36b} are absent;
   R⁴ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or substituted or unsubstituted methyl, or when [symbol] is a double bond, R⁵ is absent;
   each of R^{37a} and R^{17b} is independently hydrogen, halogen, cyano, hydroxyl, substituted or unsubstituted alkyl, substituted or unsubstituted alkenyl, or substituted or unsubstituted alkynyl, or R^{17a} and R^{17b} are joined to form an oxo (═O) group;
   each of R^{1a}, R^{1b}, R^{2a}, R^{2b}, R^{4a}, R^{4b}, R^{11a}, R^{11b}, R^{12a}, R^{12b}, R^{15a}, and R^{15b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, OC(═O)R^{D1}, —NH₂, —N(R^{D1})₂, or NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a nitrogen protecting group when attached to a nitrogen atom, or two R^{D1} groups are joined to form an substituted or unsubstituted heterocyclic ring; or any one of R^{1a} and R^{1b}, R^{2a} and R^{2b}, R^{4a} and R^{4b}, R^{11a} and R^{11b}, R^{12a} and R^{12b}, and R^{15a} and R^{15b} are joined to form an oxo (═O) group;
   each of R^{16a} and R^{16b} is each independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1} —N(R^{A1})₂, —N(R^{A1}), —CN(R^{A1})₂, —C(O)R^{A1}, —OC(═O)R^{A1}, —OC(═O)OR^{A1}, —OC(═O)SR^{A1}, —OC(═O)N(R^{A1})₂, —SC(═O)R^{A2}, —SC(═O)OR^{A1}, —SC(═O)SR^{A1}, —SC(═O)N(R^{A1})₂, —NHC(═O)R^{A1}, —NHC(═O)OR^{A1}, —NHC(═O)SR^{A1}, —NHC(═O)N(R^{A1})₂, —OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, or —S(═O)₂OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, —SO₂R^{A2}, —C(O)R^{A2}, or two R^{A1} groups are joined to form an substituted or unsubstituted heterocyclic or heteroaryl ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R¹⁹ is hydrogen, substituted or unsubstituted alkyl, substituted or unsubstituted alkenyl, or substituted or unsubstituted alkynyl;
   R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring; and
   n is 1, 2, or 3.
16a. The compound of embodiment 15a, wherein the compound is a compound of Formula 2-VIIa:

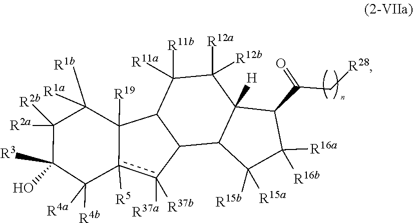

or a pharmaceutically acceptable salt thereof.
17a. The compound of any one of embodiments 1a-16a, wherein each R^{1a} and R^{6a} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —N(R^{A1}), —CN(R^{A1})₂, —C(O)R^{A1}, —OC(═O)R^{A1}, or —OC(═O)OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
18a. The compound of nay one of embodiments 1a-17a, wherein each of R^{16a} and R^{6b} is independently hydrogen, halogen, substituted or unsubstituted alkyl, substituted or unsubstituted alkenyl, —OR^{A1}, —N(R^{A1})₂, —N(R^{A1}), —CN(R^{A1})₂, —C(O)R^{A1}, —OC(═O)R^{A1}, or —OC(═O)OR^{A1}, wherein each instance of R^{A1} is independently hydrogen, substituted or unsubstituted alkyl, substituted or unsubstituted alkenyl, substituted or unsubstituted alkynyl, substituted or unsubstituted heterocyclyl, substituted or unsubstituted aryl, or substituted or unsubstituted heteroaryl.
19a. The compound of nay one of embodiments 1a-18a, wherein both R^{16a} and R^{16b} are hydrogen.
20a. The compound of nay one of embodiments 1a-19a, wherein each of R^{1a} and R^{1b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
21a. The compound of any one of embodiments 1a-20a, wherein each of R^{15a} and R^{15b} is independently hydrogen, halogen, cyano, hydroxyl, substituted or unsubstituted alkyl, substituted or unsubstituted alkenyl, —OR^{D1}, —OC(═O)R^{D1}, —NH₂, or —N(RM)₂, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
22a. The compound of any one of embodiments 1a-21a, wherein each of R^{15a} and R^{15b} is independently hydrogen, substituted or unsubstituted alkyl, —OR^{D1}, —OC(═O)R^{D1}—NH₂, —N(R^{D1})₂, or —NR^{D1}C(═O)R^{D1}, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
23a. The compound of any one of embodiments 1a-22a, wherein each of R^{15a} and R^{15b} is independently hydrogen or substituted or unsubstituted alkyl.
24a. The compound of any one of embodiments 1a-23a, wherein R^{15a} and R^{15b} are each independently hydrogen.
25a. The compound of any one of embodiments 1a-24a, wherein both R^{11a} and R^{11b} are hydrogen.
26a. The compound of any one of embodiments 1a-25a, wherein each of R^{2a} and R^{12b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1} wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
27a. The compound of any one of embodiments 1a-26a, wherein each of R² and R^{12b} is independently hydrogen, halogen, cyano, hydroxyl, substituted or unsubstituted alkyl, substituted or unsubstituted alkenyl, —OR^{D1}, —OC(═O)R^{D1}, —NH₂, or —N(R¹)₂, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
28a. The compound of any one of embodiments 1a-27a, wherein each of R² and R^{12b} is independently hydrogen, substituted or unsubstituted alkyl, —OR^{D1}, —OC(═O)R^{D1}, —NH₂, —N(R^{D1})₂, or —NR^{D1}C(═O)R^{D1}, wherein each instance of R¹ is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
29a. The compound of any one of embodiments 1a-28a, wherein each of R^{2a} and R^{12b} is independently hydrogen or substituted or unsubstituted alkyl.
30a. The compound of any one of embodiments 1a-29a, wherein each of R^{12a} and R^{12b} is independently hydrogen.
31a. The compound of any one of embodiments 1a-30a, wherein both R^{12a} and R^{12b} are hydrogen.
32a. The compound of any one of embodiments 1a-31a, wherein each of R^{11a} and R^{11b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, -OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
33a. The compound of any one of embodiments 1a-32a, wherein each of R^{11a} and R^{11b} is independently hydrogen, halogen, cyano, hydroxyl, substituted or unsubstituted alkyl, substituted or unsubstituted alkenyl, —OR^{D1}, —OC(═O)R^{D1}, —NH₂, or —N(R^{D1})₂, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
34a. The compound of any one of embodiments 1a-33a, wherein each of R^{1a} and R^{11b} is independently hydrogen, substituted or unsubstituted alkyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
35a. The compound of any one of embodiments 1a-34a, wherein each of R^{1a} and R^{1b} is independently hydrogen or substituted or unsubstituted alkyl.
36a. The compound of any one of embodiments 1a-35a, wherein each of R^{11a} and R^{11b} is independently hydrogen, C₁-C₆ alkyl, C₁-C₆ haloalkyl, C₁-C₆ alkoxy, C₁-C₆ alkoxyhalo, or —OH.
37a. The compound of any one of embodiments 1a-36a, wherein each of R^{1a} and R^{11b} is independently —CH₃, —CH₂CH₃, —OH, —OCH₃, or —CH(CH₃)₂.
38a. The compound of any one of embodiments 1a-37a, wherein R¹¹ and R^{11b} are each independently hydrogen.
39a. The compound of any one of embodiments 1a-38a, wherein both R^{11a} and R^{11b} are hydrogen.
40a. The compound of any one of embodiments 1a-39a, wherein each of R^{7a} and R^{7b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, NH₂, —N(R^{D1})₂, or —NR^{D1}C(═O)R^{D1}, wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
41a. The compound of any one of embodiments 1a-40a, wherein each of R^{7a} and R^{7b} is independently hydrogen, halogen, cyano, hydroxyl, substituted or unsubstituted alkyl, substituted or unsubstituted alkenyl, —OR^{D1}, —OC(═O)R^{D1}, —NH₂, or —N(R^{D1})₂, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
42a. The compound of any one of embodiments 1a-41a, wherein each of R^{7a} and R^{7b} is independently hydrogen, substituted or unsubstituted alkyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
43a. The compound of any one of embodiments 1a-42a, wherein each of R^{7a} and R^{7b} is independently hydrogen, C₁-C₆ alkyl, C₁-C₆ haloalkyl, C₁-C₆ alkoxy, C₁-C₆ alkoxyhalo, or —OH.
44a. The compound of any one of embodiments 1a-43a, wherein each of R^{7a} and R^{7b} is independently —CH₃, —CH₂CH₃, —OH, —OCH₃, or —CH(CH₃)₂.
45a. The compound of any one of embodiments 1a-44a, wherein each of R^{7a} and R^{7b} is independently hydrogen or substituted or unsubstituted alkyl.
46a. The compound of any one of embodiments 1a-45a, wherein each of R^{7a} and R^{7b} is independently hydrogen.
47a. The compound of any one of embodiments 1a-46a, wherein both R^{7a} or R^{7b} are hydrogen.
48a. The compound of any one of embodiments 1a-47a, wherein each of R^{6a} and R^{6b} is independently hydrogen, halogen, substituted or unsubstituted alkyl, substituted or unsubstituted alkenyl, or substituted or unsubstituted alkynyl.
49a. The compound of any one of embodiments 1a-48a, wherein both R^{6a} and R^{6b} are halogen.
50a. The compound of any one of embodiments 1a-49a, wherein both R^{6a} and R^{6b} are alkyl.
51a. The compound of any one of embodiments 1a-50a, wherein R^{6a} and R^{6b} are joined to form an oxo group.
52a. The compound of any one of embodiments 1a-51a, wherein each of R^{6a} and R^{6b} is independently hydrogen or substituted or unsubstituted alkyl.
53a. The compound of any one of embodiments 1a-52a, wherein each of R^{6a} and R^{6b} is independently hydrogen or substituted alkyl.
54a. The compound of any one of embodiments 1a-53a, wherein each of R^{6a} and R^{6b} is independently hydrogen or unsubstituted alkyl.
55a. The compound of any one of embodiments 1a-54a, wherein R^{6a} is halogen or alkyl and R^{6b} is hydrogen.
56a. The compound of any one of embodiments 1a-55a, wherein each of R^{6a} and R^{6b} is independently hydrogen.
57a. The compound of any one of embodiments 1a-56a, wherein both R^{6a} and R^{6b} are hydrogen.
58a. The compound of any one of embodiments 1a-57a, wherein R⁵ is methyl in the cis position.
59a. The compound of any one of embodiments 1a-58a, wherein R⁵ is hydrogen in the cis position.
60a. The compound of any one of embodiments 1a-59a, wherein R⁵ is hydrogen in the trans position.
61a. The compound of any one of embodiments 1a-60a, wherein each of R⁴ and R^{4b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1} wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
62a. The compound of any one of embodiments 1a-61a, wherein each of R^{4a} and R^{4b} is independently hydrogen, halogen, cyano, hydroxyl, substituted or unsubstituted alkyl, substituted or unsubstituted alkenyl, —OR^{D1}, —OC(═O)R^{D1}, —NH₂, or —N(R^{D1})₂, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
63a. The compound of any one of embodiments 1a-62a, wherein each of R^{4a} and R^{4b} is independently hydrogen, substituted or unsubstituted alkyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
64a. The compound of any one of embodiments 1a-63a, wherein each of R^{4a} and R^{4b} is independently hydrogen or substituted or unsubstituted alkyl.
65a. The compound of any one of embodiments 1a-64a, wherein each of R^{4a} and R^{4b} is independently hydrogen, C₁-C₆ alkyl, C₁-C₆ haloalkyl, C₁-C₆ alkoxy, C₁-C₆ alkoxyhalo, or —OH.
66a. The compound of any one of embodiments 1a-65a, wherein each of R^{4a} and R^{4b} is independently —CH₃, —CH₂CH₃, —OH, —OCH₃, or —CH(CH₃)₂.

67a. The compound of any one of embodiments 1a-66a, wherein each of R^{4a} and R^{4b} is independently hydrogen or substituted or unsubstituted alkyl.
68a. The compound of any one of embodiments 1a-67a, wherein R^{4a} and R^{4b} are each independently hydrogen.
69a. The compound of any one of embodiments 1a-68a, wherein both R^{4a} and R^{4b} are hydrogen.
70a. The compound of any one of embodiments 1a-69a, wherein R³ is substituted or unsubstituted alkyl, substituted or unsubstituted alkenyl, or substituted or unsubstituted alkynyl.
71a. The compound of any one of embodiments 1a-70a, wherein R³ is substituted or unsubstituted carbocyclyl, substituted or unsubstituted heterocyclyl, substituted or unsubstituted aryl, or substituted or unsubstituted heteroaryl.
72a. The compound of any one of embodiments 1a-71a, wherein R³ is substituted or unsubstituted alkyl.
73a. The compound of any one of embodiments 1a-72a, wherein R³ is substituted alkyl.
74a. The compound of any one of embodiments 1a-73a, wherein R³ is unsubstituted alkyl.
75a. The compound of any one of embodiments 1a-74a, wherein R³ is methyl.
76a. The compound of any one of embodiments 1a-75a, wherein R³ is —CH₂OCH₃ or —CH₂OCH₂CH₃.
77a. The compound of any one of embodiments 1a-76a, wherein each of R^{2a} and R^{2b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1} wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
78a. The compound of any one of embodiments 1a-77a, wherein each of R^{2a} and R^{2b} is independently hydrogen, halogen, cyano, hydroxyl, substituted or unsubstituted alkyl, substituted or unsubstituted alkenyl, —OR^{D1}, —OC(═O)R^{D1}, —NH₂, or —N(R^{D1})₂, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
79a. The compound of any one of embodiments 1a-78a, wherein each of R^{2a} and R^{2b} is independently hydrogen, substituted or unsubstituted alkyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
80a. The compound of any one of embodiments 1a-79a, wherein each of R^{2a} and R^{2b} is independently hydrogen or substituted or unsubstituted alkyl.
81a. The compound of any one of embodiments 1a-80a, wherein each of R^{2a} and R^{2b} is independently hydrogen, C₁-C₆ alkyl, C₁-C₆ haloalkyl, C₁-C₆ alkoxy, C₁-C₆ alkoxyhalo, or —OH.
82a. The compound of any one of embodiments 1a-8a, wherein each of R^{2a} and R^{2b} is independently —CH₃, —CH₂CH₃, —OH, —OCH₃, or —CH(CH₃)₂.
83a. The compound of any one of embodiments 1a-82a, wherein both R^{2a} and R^{2b} are hydrogen.
84a. The compound of any one of embodiments 1a-83a, wherein R^{2a} and R^{2b} are each independently hydrogen.
85a. The compound of any one of embodiments 1a-84a, wherein each of R^{1a} and R^{1b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1} wherein each instance of RD is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
86a. The compound of any one of embodiments 1a-85a, wherein each of R^{1a} and R^{1b} is independently hydrogen, halogen, cyano, hydroxyl, substituted or unsubstituted alkyl, substituted or unsubstituted alkenyl, —OR^{D1}, —OC(═O)R^{D1}, —NH₂, or —N(R^{D1})₂, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
87a. The compound of any one of embodiments 1a-86a, wherein each of R^{1a} and R^{1b} is independently hydrogen, substituted or unsubstituted alkyl, —OR^{D1}, —OC(═O)R^{D1}, —NH₂, —N(R^{D1})₂, or —NR^{D1}C(═O)R^{D1}, wherein each instance of R^{D1} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
88a. The compound of any one of embodiments 1a-87a, wherein each of R^{1a} and R^{1b} is independently hydrogen or substituted or unsubstituted alkyl.
89a. The compound of any one of embodiments 1a-88a, wherein each of R^{1a} and R^{1b} is independently hydrogen, C₁-C₆ alkyl, C₁-C₆ haloalkyl, C₁-C₆ alkoxy, C₁-C₆ alkoxyhalo, or —OH.
90a. The compound of any one of embodiments 1-89, wherein each of R^{1a} and R^{1b} is independently —CH₃, —CH₂CH₃, —OH, —OCH₃, or —CH(CH₃)₂.

91a. The compound of any one of embodiments 1a-90a, wherein both R^{1a} and R^{1b} are hydrogen.
92a. The compound of any one of embodiments 1a-91a, wherein R^{1a} and R^{1b} are each independently hydrogen.
93a. The compound of any one of embodiments 1a-92a, wherein R¹⁹ is substituted alkyl.
94a. The compound of any one of embodiments 1a-93a, wherein R¹⁹ is unsubstituted alkyl.
95a. The compound of any one of embodiments 1a-94a, wherein R¹⁹ is methyl.
96a. The compound of any one of embodiments 1a-95a, wherein R¹⁹ is —CH₂OCH₃.
97a. The compound of any one of embodiments 1-96, wherein R¹⁹ is —OCH₃.
98a. The compound of any one of embodiments 1a-97a, wherein R¹⁹ is ethyl.
99a. The compound of any one of embodiments 1a-98a, wherein R¹⁹ is hydrogen.
100a. The compound of any one of embodiments 1a-99a, wherein n is 2 or 1.
101a. The compound of any one of embodiments 1a-100a, wherein n is 2.
102a. The compound of any one of embodiments 1a-101a, wherein n is 1.
103a. The compound of any one of embodiments 1a-102a, wherein [symbol] is a single bond.
104a. The compound of any one of embodiments 1a-103a, wherein [symbol] is a double bond.
105a. The compound of any one of embodiments 1a-104a, wherein the compound is of Formula 2-Ic:

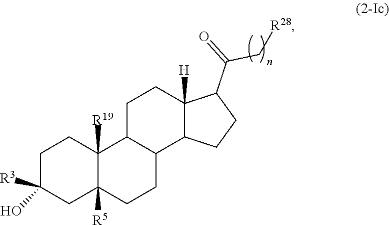

or a pharmaceutically acceptable salt thereof.
106a. The compound of any one of embodiments 1a-105a, wherein the compound is of Formula 2-Id:

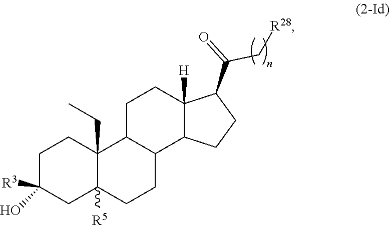

or a pharmaceutically acceptable salt thereof.
107a. The compound of any one of embodiments 1a-106a, wherein the compound is of Formula 2-Ie:

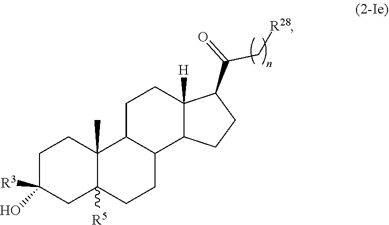

or a pharmaceutically acceptable salt thereof.

108a. The compound of any one of embodiments 1a-107a, wherein the compound is of Formula 2-If:

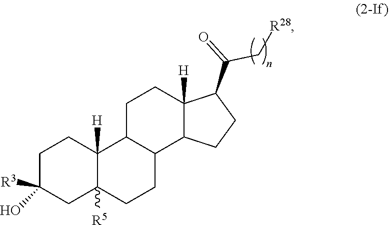

or a pharmaceutically acceptable salt thereof.
109a. The compound of any one of embodiments 1a-108a, wherein the compound is of Formula 2-Ig, Formula 2-Ig-II, or Formula 2-Ih:

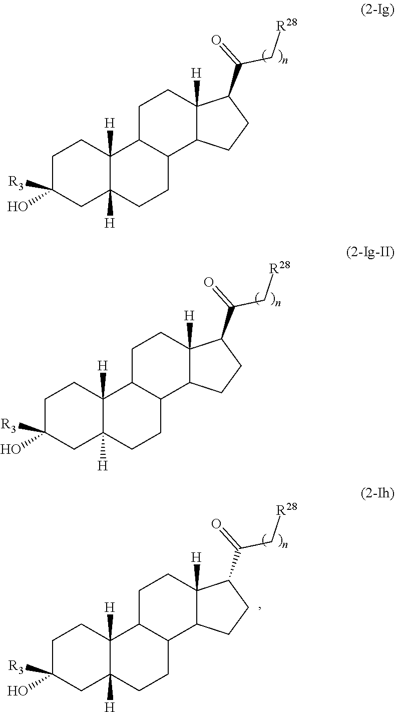

or a pharmaceutically acceptable salt thereof.
110a. The compound of any one of embodiments 1a-109a, wherein the compound is of Formula 2-Iga or Formula 2-Iha:

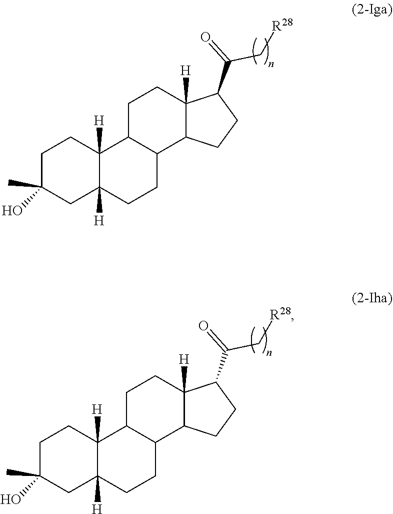

or a pharmaceutically acceptable salt thereof.
111a. The compound of any one of embodiments 1a-110a, wherein the compound is of Formula 2-Igb or Formula 2-Ihb:

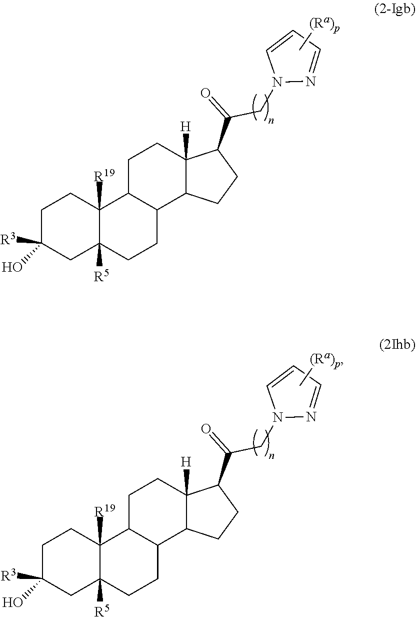

or a pharmaceutically acceptable salt thereof;
wherein:
   each instance of R^{a} is independently hydrogen, halogen, —NO₂, —CN, —OR^{D4}, —N(R^{D4})₂, —C(═O)R^{D4}, —C(═O)OR^{D4}, —C(═O)N(R^{D4})₂, —OC(═O)R^{D4}, —OC(═O)OR^{D4}, —N(R^{D4})C(═O)R^{D4}, —OC(═O)N(R^{D4})₂, —N(R^{D4})C(═O)OR^{D4}, —S(═O)₂R^{D4}, —S(═O)₂OR^{D4}, —OS(═O)₂R^{D4}, —S(═O)₂N(R^{D4})₂, or —N(R^{D4})S(═O)₂R^{D4}, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl;
   each instance of R^{D4} is independently hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl, an oxygen protecting group when attached to oxygen, a nitrogen protecting group when attached to nitrogen, or two R^{D4} groups are taken with the intervening atoms to form a substituted or unsubstituted heterocyclic ring;
   n is 1 or 2; and
   p is 1, 2, or 3.
112a. The compound of any one of embodiments 1a-111a, wherein the compound is of Formula 2-Igc:

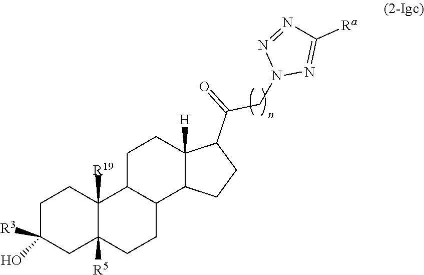

or a pharmaceutically acceptable salt thereof;
wherein:
   R^{a} is hydrogen, halogen, —NO₂, —CN, —OR^{D4}, —N(R^{D4})₂—C(═O)R^{D4}, —C(═O)OR^{D4}, —C(═O)N(R^{D4})₂, —OC(═O)R^{D4}, —OC(═O)OR^{D4}, —N(R^{D4})C(═O)R^{D4}, —OC(═O)N(R^{D4})₂, —N(R^{D4})C(═O)OR^{D4}, —S(═O)₂R^{D4}, —S(═O)₂OR^{D4}, —OS(═O)₂R^{D4}, —S(═O)₂N(R^{D4})₂, or —N(R^{D4})S(═O)₂R^{D4}, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl;
   each instance of R^{D4} is independently hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl, an oxygen protecting group when attached to oxygen, a nitrogen protecting group when attached to nitrogen, or two R^{D4} groups are taken with the intervening atoms to form a substituted or unsubstituted heterocyclic ring; and
   n is 1, 2, or 3.
113a. The compound of any one of embodiments 1a-112a, wherein the compound is of Formula 2-Igd:

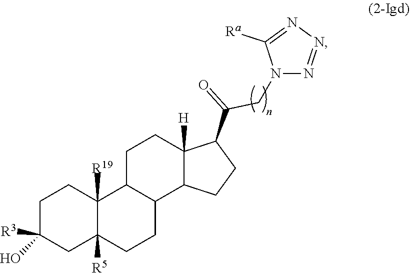

or a pharmaceutically acceptable salt thereof;
wherein:
   R^{a} is independently hydrogen, halogen, —NO₂, —CN, —OR^{D4}, —N(R^{D4})₂, —C(═O)R^{D4}, —C(═O)OR^{D4}, —C(═O)N(R^{D4})₂ —OC(═O)R^{D4}, —OC(═O)OR^{D4}, —N(R^{D4})C(═O)R^{D4}, —OC(═O)N(R^{D4})₂, —N(R^{D4})C(═O)OR^{D4}, —S(═O)₂R^{D4}, —S(═O)₂OR^{D4}, —OS(═O)₂R^{D4}, —S(═O)₂N(R^{D4})₂, or —N(R^{D4})S(═O)₂R^{D4}, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl; each instance of R^{D4} is independently hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl, an oxygen protecting group when attached to oxygen, a nitrogen protecting group when attached to nitrogen, or two R^{D4} groups are taken with the intervening atoms to form a substituted or unsubstituted heterocyclic ring; and
   n is 1, 2, or 3.
114a. The compound of any one of embodiments 1a-113a, wherein R²⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl.
115a. The compound of any one of embodiments 1a-114a, wherein R²⁸ is hydrogen, substituted or unsubstituted alkyl, substituted or unsubstituted heteroaryl.
116a. The compound of any one of embodiments 1a-115a, wherein R²⁸ is methyl.
117a. The compound of any one of embodiments 1a-116a, wherein R²⁸ is hydrogen.
118a. The compound of any one of embodiments 1a-117a, wherein R²⁸ is selected from the group consisting of:

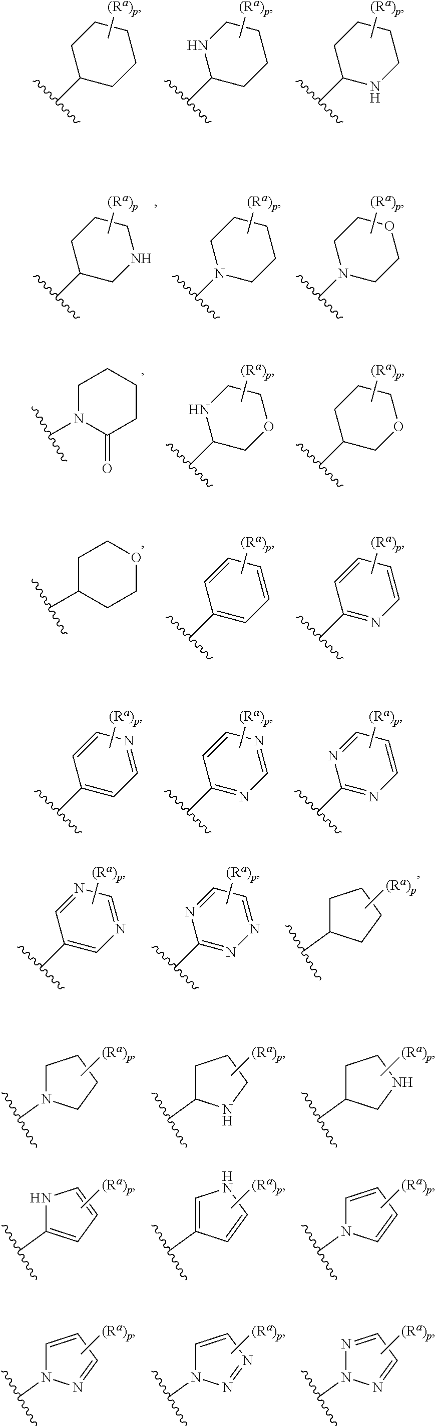

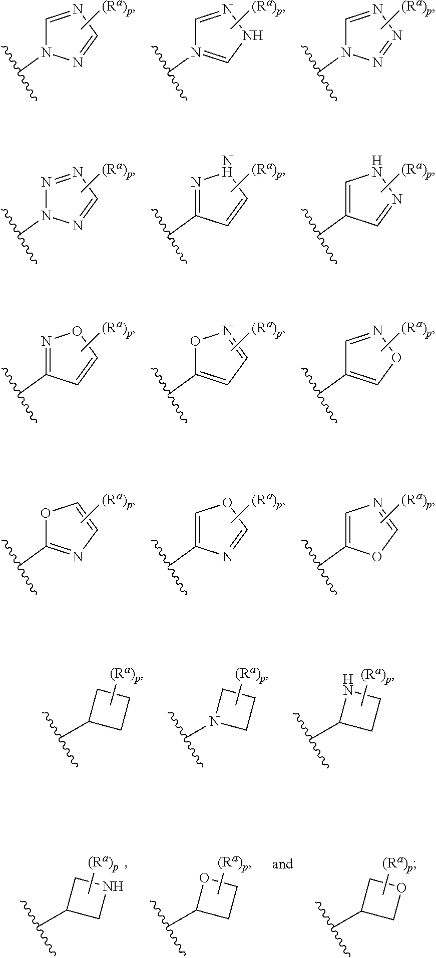

wherein:
   each instance of R^{a} is independently hydrogen, halogen, —NO₂, —CN, —OR^{D4}, —N(R^{D4})₂, —C(═O)R^{D4}, —C(═O)OR^{D4}, —C(═O)N(R^{D4})₂, —OC(═O)R^{D4}, —OC(═O)OR^{D4}, —N(R^{D4})C(═O)R^{D4}, —OC(═O)N(R^{D4})₂, —N(R^{D4})C(═O)OR^{D4}, —S(═O)₂R^{D4}, —S(═O)₂OR^{D4}, —OS(═O)₂R^{D4}, —S(═O)₂N(R^{D4})₂, or —N(R^{D4})S(═O)₂R^{D4}, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl;
   each instance of R^{D4} is independently hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl, an oxygen protecting group when attached to oxygen, a nitrogen protecting group when attached to nitrogen, or two R^{D4} groups are taken with the intervening atoms to form a substituted or unsubstituted heterocyclic ring; and
   p is an integer selected from 0 to 11.
119a. The compound of any one of embodiments 1a-118a, wherein R²⁸ is selected from the group consisting of:

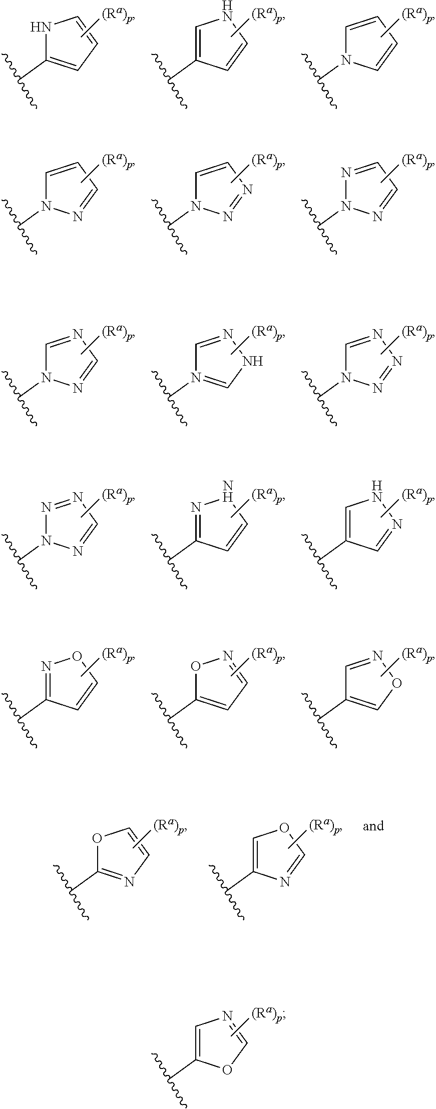

wherein R^{a} and p is as defined in embodiment 118a.
120a. The compound of any one of embodiments 1a-119a, wherein R²⁸ is

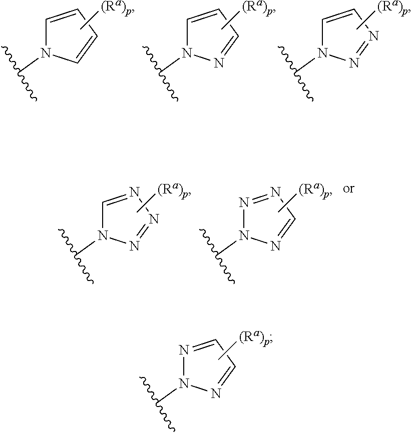

wherein R^{a} and p is as defined in embodiment 118a.
121a. The compound of any one of embodiments 1a-120a wherein R²⁸ is

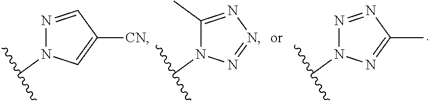

122a. The compound of any one of embodiments 1a-12′1a, wherein each of R^{37a} and R^{37b} is independently hydrogen, halogen, substituted or unsubstituted alkyl, substituted or unsubstituted alkenyl, or substituted or unsubstituted alkynyl.
123a. The compound of any one of embodiments 1a-122a, wherein each of R^{36a} and R^{36b} is independently hydrogen, halogen, substituted or unsubstituted alkyl, substituted or unsubstituted alkenyl, or substituted or unsubstituted alkynyl.
124a. The compound of any one of embodiments 1a-123a, wherein each of R^{35a} and R^{35b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NRPmC(═O)R^{D1} wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
125a. The compound of any one of embodiments 1a-124a, wherein each of R^{34a} and R^{34b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1} wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
126a. The compound of any one of embodiments 1a-125a, wherein each of R^{33a} and R^{33b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NR^{D1}C(═O)R^{D1} wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
127a. The compound of any one of embodiments 1a-126a, wherein each of R^{32a} and R^{32b} is independently hydrogen, halogen, cyano, hydroxyl, substituted or unsubstituted alkyl, substituted or unsubstituted alkenyl, substituted or unsubstituted heterocyclyl, or substituted or unsubstituted alkynyl, —OR^{D1}, —OC(═O)R^{D1}, —NH₂, —N(R^{D1})₂, or —NRPC(═O)R^{D1} wherein each instance of R^{D1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
128a. The compound of any one of embodiments 1a-127a, wherein each R^{31a} and R^{31b} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, N(R^{A1})₂, N(R^{A1}), —CN(R^{A1})₂, —C(O)R^{A1}, —OC(═O)R^{A1}, or —OC(═O)OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
129a. The compound of any one of embodiments 1a-128a, wherein each of R^{30a} and R^{30b} is independently hydrogen, halogen, cyano, hydroxyl, substituted or unsubstituted alkyl, substituted or unsubstituted alkenyl, —OR^{D1}, —OC(═O)R^{D1}, —NH₂, or —N(R^{D1})₂, wherein each instance of R¹ is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl.
130a. The compound of any one of embodiments 1a-129a, wherein each R^{29a} and R^{29b} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —SR^{A1}, —N(R^{A1})₂, —N(R^{A1}), —CN(R^{A})₂, —C(O)R^{A1}, —OC(═O)R^{A1}, or —OC(═O)OR^{A1}, wherein each instance of R^{A1} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl.
131a. The compound of any one of embodiments 1a-130a, wherein t is 2.
132a. The compound of any one of embodiments 1a-131a, wherein t is 1.
133a. The compound of any one of embodiments 1a-132a, wherein m is 3.
134a. The compound of any one of embodiments 1a-133a, wherein m is 2.
135a. The compound of any one of embodiments 1a-134a, wherein the compound is selected from the group consisting of:

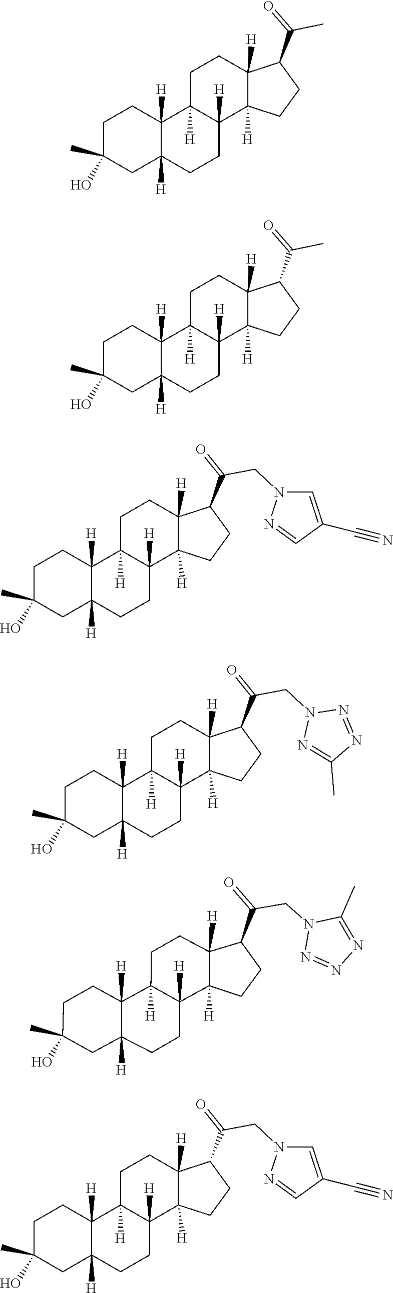

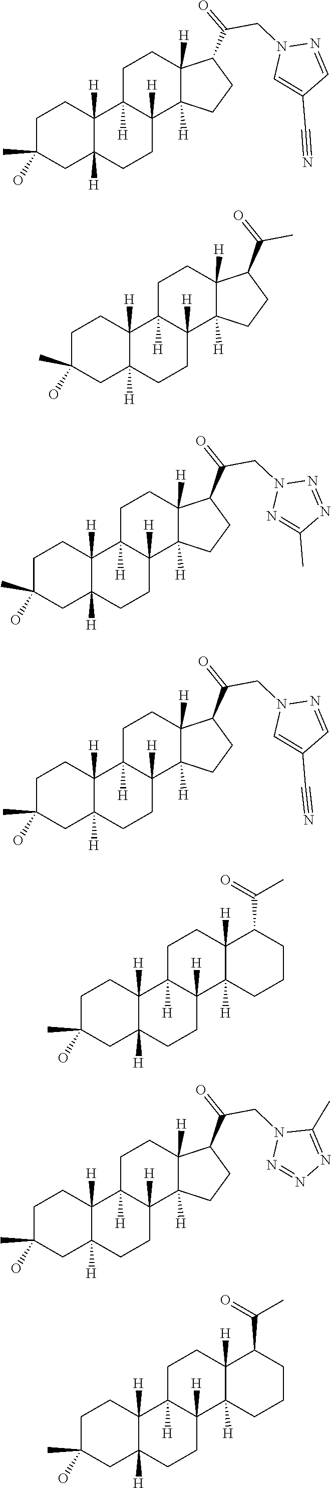

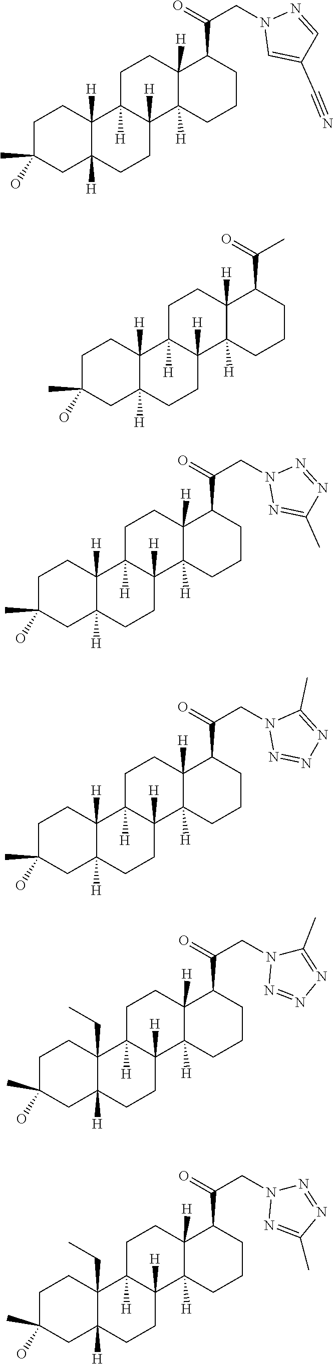

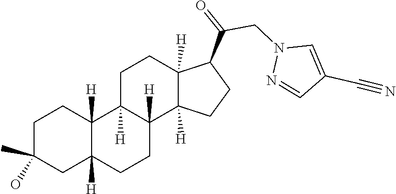

136a. A pharmaceutical composition comprising a compound of any one of embodiments 1a-135a or a pharmaceutically acceptable salt thereof, and a pharmaceutically acceptable excipient.
137a. A method of modulating a GABA_{A} receptor in a subject in need thereof, comprising administering to the subject a therapeutically effective amount of a compound of any one of embodiments 1a-136a or a pharmaceutically acceptable salt thereof, or a pharmaceutical composition of embodiment 136a.
138a. A method of treating a CNS-related disorder in a subject in need thereof, comprising administering to the subject an effective amount of a compound of any one of embodiments 1a-136a or a pharmaceutically acceptable salt thereof.
139a. The method of embodiment 138a, wherein the CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, or status epilepticus.
140a. The method of embodiment 139a, wherein the CNS-related disorder is a mood disorder.
141a. The method of embodiment 140a, wherein the CNS-related disorder is depression.
142a. The method of embodiment 141a, wherein the CNS-related disorder is postpartum depression.
143a. The method of embodiment 142a, wherein the CNS-related disorder is major depressive disorder.
144a. The method of embodiment 143a, wherein the major depressive disorder is moderate major depressive disorder.
145a. The method of embodiment 144a, wherein the major depressive disorder is severe major depressive disorder.

The present disclosure includes the following embodiments numbered 1b-104b:
1b. A compound of Formula 3-T:

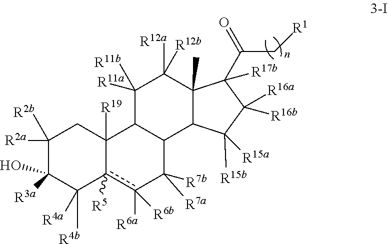

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R^{2a}, R^{2b}, R^{4a}, R^{4b}, R^{7a}, R^{7b}, R^{11a}, R^{11b}, R^{12a}, R^{12b} or R^{4b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁸ is substituted or unsubstituted alkyl;
   R¹⁹ is substituted or unsubstituted C₃-C₆ carbocyclyl or substituted or unsubstituted aryl; and
   n is 0, 1 or 2.
2b. The compound of embodiment 1b, wherein the compound is a compound of Formula 3-IIa or Formula 3-IIb:

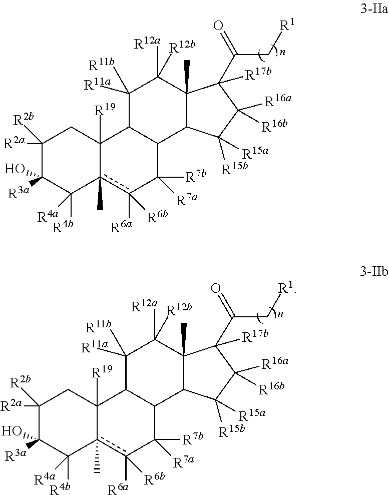

3b. A compound of Formula 3-III:

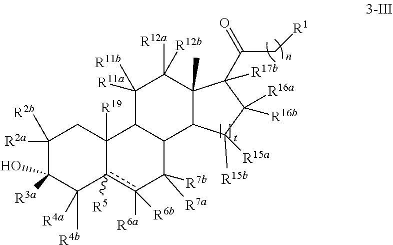

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂—S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂—S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted C₃-C₆ carbocyclyl or substituted or unsubstituted aryl;
   n is 0, 1 or 2 and
   t is 2 or 3.
4b. A compound of Formula 3-IV:

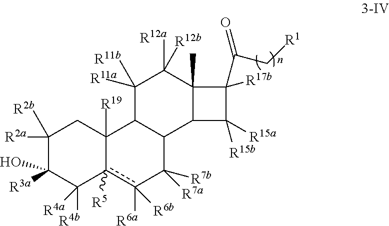

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, -OR^{A1}, —N(R^{A1})₂, —SR^{A1}, —C(═O)R^{A1}, —C(═O)OR^{A1}, —C(═O)SR^{A1}, —C(═O)N(R^{A})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a} and R^{15b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, -OR^{C3}, —N(R^{C3})₂, —SR^{C3}, —C(═O)R^{C3}, —C(═O)OR^{C3}, —C(═O)SR^{C3}, —C(═O)N(R^{C3})₂ —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted C₃-C₆ carbocyclyl or substituted or unsubstituted aryl; and
   n is 0, 1 or 2.
5b. A compound of Formula 3-Va or Formula 3-Vb:

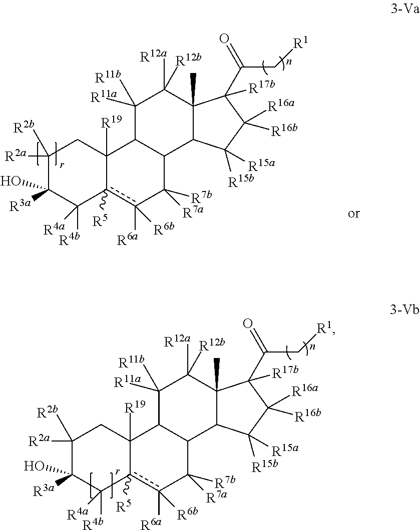

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted C₃-C₆ carbocyclyl or substituted or unsubstituted aryl;
   n is 0, 1 or 2; and
   r is 2 or 3.
6b. A compound of Formula 3-VI:

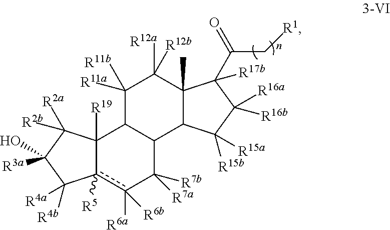

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂—OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1}, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R^{2a}, R^{2b}, R^{7a}, R^{7b}, R^{11a}, R^{11b}, R^{12a}, R^{12b} or R^{7b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂—S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R³, is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3} —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted C₃-C₆ carbocyclyl or substituted or unsubstituted aryl;
   n is 0, 1 or 2.
7b. A compound of Formula 3-VII:

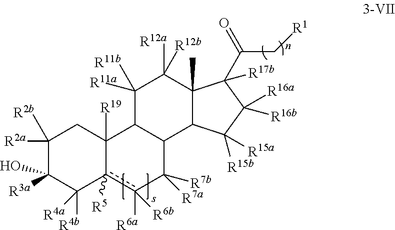

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3} —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted C₃-C₆ carbocyclyl or substituted or unsubstituted aryl;
   n is 0, 1 or 2; and
   s is 2.
8b. A compound of Formula 3-VIII:

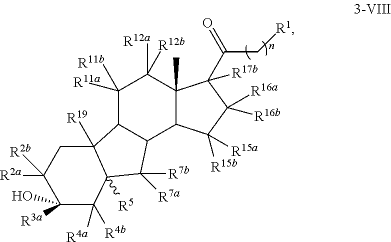

or a pharmaceutically acceptable salt thereof;
wherein:
   [symbol] represents a single or double bond, provided if a double bond is present, then one of R^{6a} or R^{b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂ —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{2a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂—S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted C₃-C₆ carbocyclyl or substituted or unsubstituted aryl; and
   n is 0, 1 or 2.
9b. A compound of Formula 3-TX:

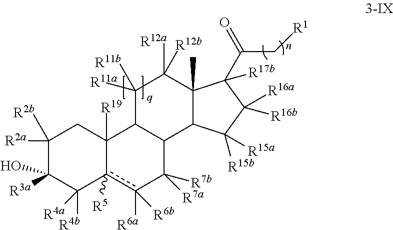

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, -OR^{A1}, —N(R^{A1})₂, —SR^{A1}, —C(═O)R^{A1}, —C(═O)OR^{A1}, —C(═O)SR^{A1}, —C(═O)N(R^{A})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] a double bond, R⁵ is absent;
   each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, -OR^{C3}, —N(R^{C3})₂, —SR^{C3}, —C(═O)R^{C3}, —C(═O)OR^{C3}, —C(═O)SR^{C3}, —C(═O)N(R^{C3})₂ —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted C₃-C₆ carbocyclyl or substituted or unsubstituted aryl;
   n is 0, 1 or 2; and
   q is 2.
10b. A compound of Formula 3-Ia:

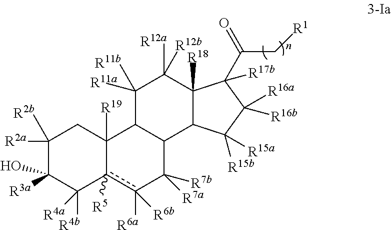

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A}t)₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁸ is substituted or unsubstituted alkyl;
   R¹⁹ is substituted or unsubstituted heterocyclyl or substituted or unsubstituted heteroaryl; and
   n is 0, 1 or 2.
11b. The compound of embodiment 10b, wherein the compound is a compound of Formula 3-IIaa or Formula 3-IIba:

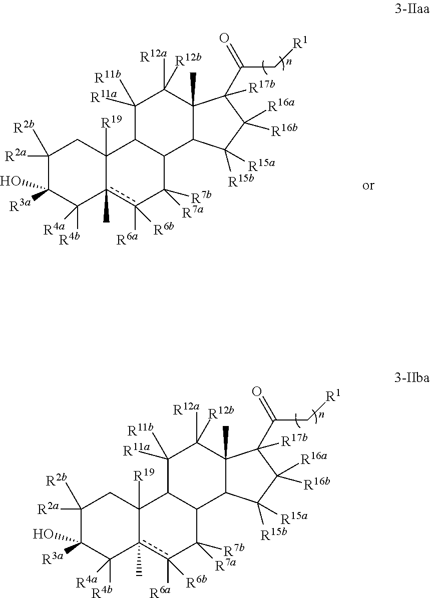

12b. A compound of Formula 3-IIIa:

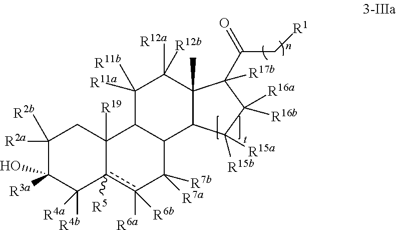

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R², R^{2b}, R^{4a}, R^{4b}, R^{7a}, R^{7b}, R^{11a}, R^{11b}, R^{12a}, R^{12b} or R⁴, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A}], —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(RAJ)₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂ —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted heterocyclyl or substituted or unsubstituted heteroaryl;
   n is 0, 1 or 2 and
   t is 2 or 3.
13b. A compound of Formula 3-IVa:

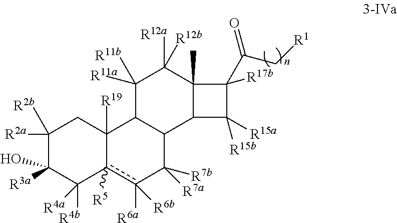

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, -OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R^{2a}, R^{2b}, R^{4a}, R^{4b}, R^{7a}, R⁷, R^{11a}, R^{11b}, R^{12a}, R^{12b} or R¹⁷, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R³, is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a} and R^{15b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂₀R^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted heterocyclyl or substituted or unsubstituted heteroaryl; and
   n is 0, 1 or 2.
14b. A compound of Formula 3-Vac or Formula 3-Vacc:

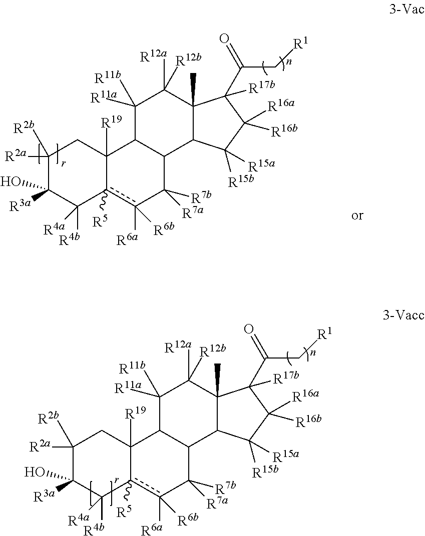

or a pharmaceutically acceptable salt thereof;
wherein:
   [symbol] represents a single or double bond, provided if a double bond is preent, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂—OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted heterocyclyl or substituted or unsubstituted heteroaryl;
   n is 0, 1 or 2; and
   r is 2 or 3.
15b. A compound of Formula 3-VIac:

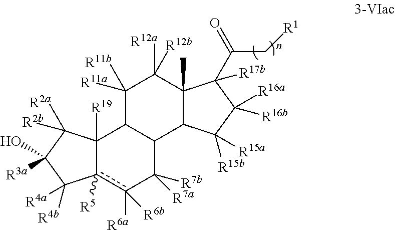

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂—S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R^{2a}, R^{2b}, R^{7a}, R^{7b}, R^{11a}, R^{11b}, R^{11a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R⁶ and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted heterocyclyl or substituted or unsubstituted heteroaryl;
   n is 0, 1 or 2.
16b. A compound of Formula 3-VIIac:

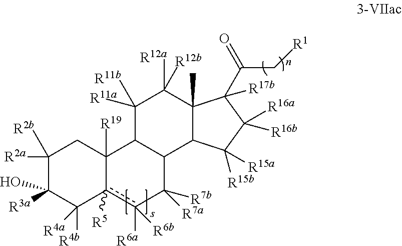

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, -OR^{A1}, —N(R^{A1})₂, —SR^{A1}, —C(═O)R^{A1}, —C(═O)OR^{A1}, —C(═O)SR^{A1}, —C(═O)N(R^{A})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂—OC(═O)SR^{A1}, —OS(═O)₂R^{A1}, —OS(═O)₂OR^{A1}, —OS(O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted heterocyclyl or substituted or unsubstituted heteroaryl;
   n is 0, 1 or 2; and
   s is 2.
17b. A compound of Formula 3-VIIIac:

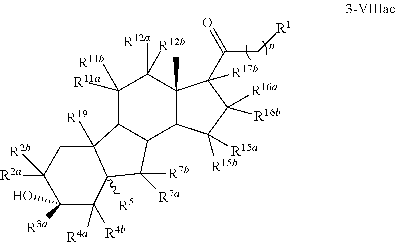

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, -OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R^{2a}, R^{2b}, R^{4a}, R^{4b}, R^{7a}, R^{7b}, R^{11a}, R^{11b}, R^{12a}, R^{12b} or R^{17b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A}], —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(RAJ)₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R¹ is absent;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted heterocyclyl or substituted or unsubstituted heteroaryl; and
   n is 0, 1 or 2.
18b. A compound of Formula 3-IXac:

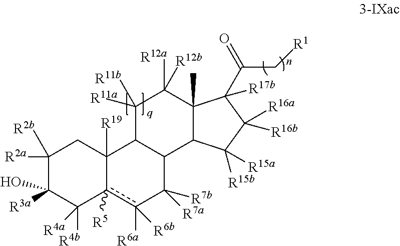

or a pharmaceutically acceptable salt thereof;
wherein:
   [symbol] represents a single or double bond, provided if a double bond is present, then one of R^{6a} or R^{6b} is absent;
   R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, -OR^{A1}, —N(R^{A1})₂, —SR^{A1}, —C(═O)R^{A1}, —C(═O)OR^{A1}, —C(═O)SR^{A1}, —C(═O)N(R^{A})₂, —OC(═O)R^{A1}, —OC(═O)OR^{A1}, —OC(═O)N(R^{A1})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   each of R^{2a}, R^{2b}, R^{4a}, R^{4b}, R^{7a}, R^{7b}, R^{11a}, R^{11b}, R^{12a}, R^{12b} or R^{4b}, is independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —C(═O)R^{A1}, —C(═O)OR^{A1}, —C(═O)SR^{A1}, —C(═O)N(R^{A1})₂, —OC(═O)R^{A1}, —OC(═O)OR^{A1}, —OC(═O)N(R^{A})₂, —OC(═O)SR^{A1}, —OS(═O)₂R^{A1}, —OS(═O)₂OR^{A1}, —OS(═O)₂N(R^{A1})₂, —N(R^{A1})C(═O)R^{A1}, —N(R^{A1})C(═NR^{A1})R^{A1}, —N(R^{A1})C(═O)OR^{A1}, —N(R^{A1})C(═O)N(R^{A1})₂, —N(R^{A1})C(═NR^{A1}) N(R^{A1})₂, —N(R^{A1})S(═O)₂R^{A1}, —N(R^{A1})S(═O)₂OR^{A1}, —N(R^{A1})S(═O)₂N(R^{A1})₂, —SC(═O)R^{A1}, —SC(═O)OR^{A1}, —SC(═O)SR^{A1}, —SC(═O)N(R^{A1})₂, —S(═O)₂R^{A1}, —S(═O)₂OR^{A1}, or —S(═O)₂N(R^{A1})₂, wherein each instance of R^{A1} is independently selected from hydrogen, substituted or unsubstituted alkyl, substituted or unsubstituted alkenyl, substituted or unsubstituted alkynyl, substituted or unsubstituted carbocyclyl, or substituted or unsubstituted heterocyclyl, substituted or unsubstituted aryl, substituted or unsubstituted heteroaryl, an oxygen protecting group when attached to oxygen, a nitrogen protecting group when attached to nitrogen, a sulfur protecting group when attached to sulfur, or two R^{A1} groups are taken with the intervening atoms to form a substituted or unsubstituted heterocyclic ring;
   R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
   R⁵ is hydrogen or methyl; when [symbol] is a double bond, R⁵ is absent;
   each of R^{6a} and R^{6b} is hydrogen, halogen, —CN, —NO₂, —OH, substituted or unsubstituted alkyl, substituted or unsubstituted alkenyl, or substituted or unsubstituted alkynyl; or R^{6a} and R^{6b} are joined to form an oxo (═O) group;
   each of R^{15a}, R^{15b}, R^{16a} and R^{16b} is each independently hydrogen, halogen, —CN, —NO₂, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{C3}, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl, an oxygen protecting group when attached to oxygen, a nitrogen protecting group when attached to nitrogen, a sulfur protecting group when attached to sulfur, or two R^{C3} groups are taken with the intervening atoms to form a substituted or unsubstituted heterocyclic ring;
   R¹⁹ is substituted or unsubstituted heterocyclyl or substituted or unsubstituted heteroaryl;
   n is 0, 1 or 2; and
   q is 2.
19b. The compound of embodiment 10b-18b, wherein R¹⁹ is not

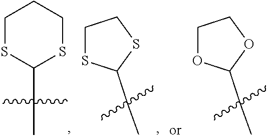

20b. The compound of any one of embodiments 1b-19b, wherein R¹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl.
21b. The compound of any one of embodiments 1b-19b, wherein R¹ is substituted or unsubstituted carbocyclyl, substituted or unsubstituted heterocyclyl, substituted or unsubstituted aryl, substituted or unsubstituted heteroaryl.
22b. The compound of any one of embodiments 1b-19b, wherein R¹ is substituted carbocyclyl, substituted heterocyclyl, substituted aryl, or substituted heteroaryl, wherein each is further substituted with substituted carbocyclyl, substituted heterocyclyl, substituted aryl, or substituted heteroaryl.
23b. The compound of any one of embodiments 1b-19b, wherein R¹ is selected from the group consisting of:

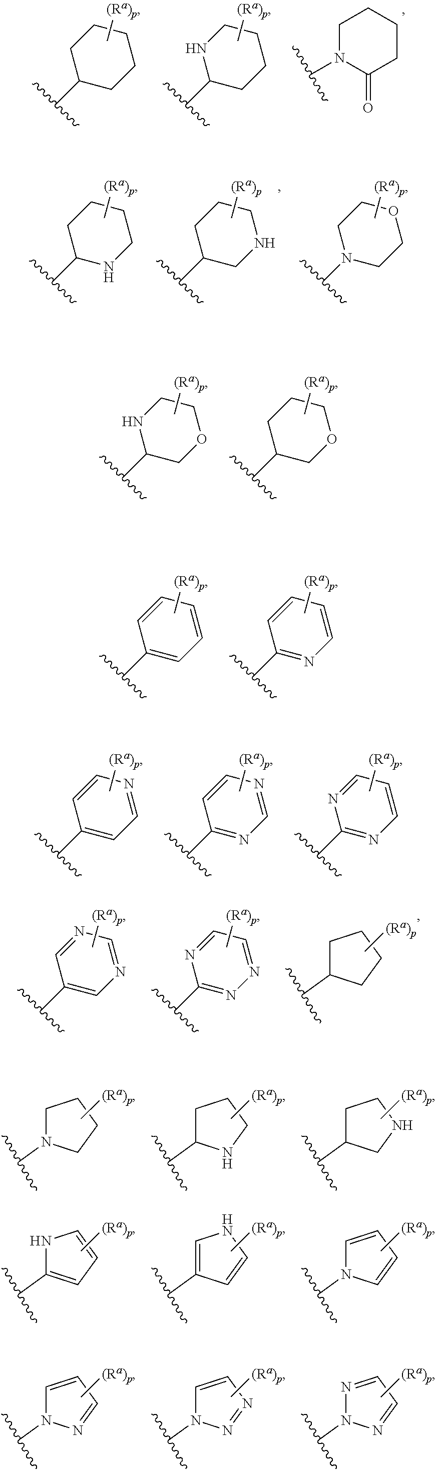

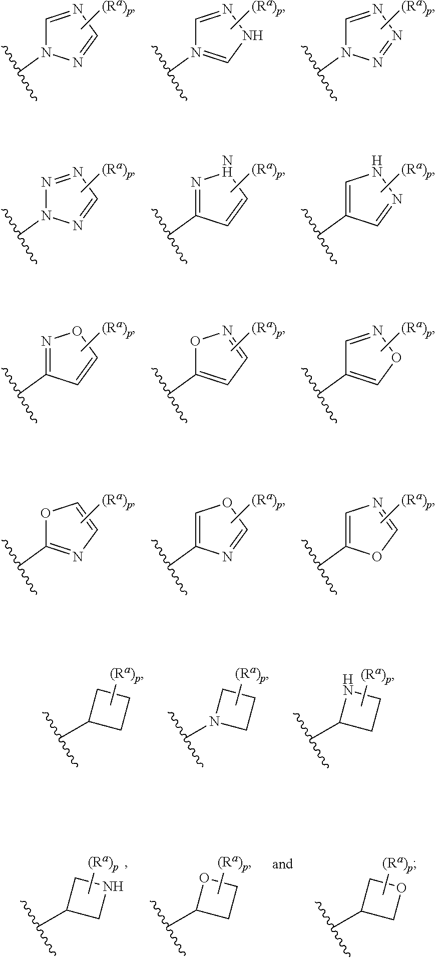

wherein:
   each instance of R^{a} is independently hydrogen, halogen, —NO₂, —CN, —OR^{D4}, —N(R^{D4})₂, —C(═O)R^{D4}, —C(═O)OR^{D4}, —C(═O)N(R^{D4})₂, —OC(═O)R^{D4}, —OC(═O)OR^{D4}, —N(R^{D4})C(═O)R^{D4}, —OC(═O)N(R^{D4})₂, —N(R^{D4})C(═O)OR^{D4}, —S(═O)₂R^{D4}, —S(═O)₂OR^{D4}, —OS(═O)₂R^{D4}, —S(═O)₂N(R^{D4})₂, or —N(R^{D4})S(═O)₂R^{D4}, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl;
   each instance of R^{D4} is independently hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl, an oxygen protecting group when attached to oxygen, a nitrogen protecting group when attached to nitrogen, or two R^{D4} groups are taken with the intervening atoms to form a substituted or unsubstituted heterocyclic ring; and
   p is an integer selected from 0 to 11.
24b. The compound of any one of embodiments 1b-19b, wherein R¹ is selected from the group consisting of:

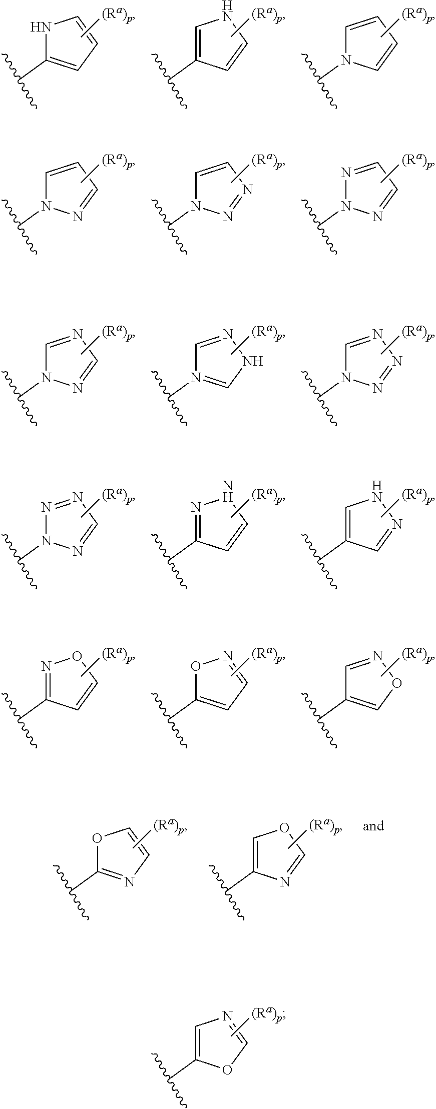

wherein R^{a} and p is as defined in embodiment 23b.
25b. The compound of any one of embodiments 1b-24b, wherein R² and R^{2b} is each independently hydrogen, halogen, —CN, —NO₂, substituted or unsubstituted alkyl, substituted or unsubstituted alkenyl, substituted or unsubstituted alkynyl, —OR^{E5}, —OC(═O)R^{E5}, —OS(═O)₂OR^{E5}, —N(R^{E5})₂, or —N(R^{E5})C(═O)R^{E5}, —N(R^{E5})S(═O)₂R^{E5}, —N(R^{E5})S(═O)₂OR^{E5}; wherein each instance of R^{E5} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, or two R^{E}5 groups are taken with the intervening atoms to form a substituted or unsubstituted heterocyclic ring.
26b. The compound of any one of embodiments 1b-24b, wherein R^{2a} and R^{2b} is each independently hydrogen, halogen, —CN, —NO₂, —OR^{F6}, —OC(═O)R^{F6}, —N(R^{F6})₂, or —N(R^{F6})C(═O)R^{F6}; wherein each instance of R^{F6} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, or two R^{F6} groups are taken with the intervening atoms to form a substituted or unsubstituted heterocyclic ring.
27b. The compound of any one of embodiments 1b-24b, wherein R^{2a} and R^{2b} are independently hydrogen, —OH, or substituted or unsubstituted C₁₋₆ alkyl.
28b. The compound of any one of embodiments 1b-24b, wherein each of R^{2a} and R^{2b} are independently hydrogen, —OH, C₁₋₆ alkyl, C₁₋₆ haloalkyl, C₁₋₆ alkoxy, or C₁₋₆ alkoxyhalo.
29b. The compound of any one of embodiments 1b-24b, wherein R^{2a} and R^{2b} are independently —CH₃, —CH₂CH₃, —OH, —OCH₃, or —CH(CH₃)₂.
30b. The compound of any one of embodiments 1b-24b, wherein R^{2a} and R^{2b} are both hydrogen.
31b. The compound of any one of embodiments 1b-24b, wherein R^{2a} and R^{2b} are joined to form an oxo (═O) group.
32b. The compound of any one of embodiments 1b-31b, wherein R^{3a} is hydrogen, substituted or unsubstituted alkyl, substituted or unsubstituted alkenyl, or substituted or unsubstituted alkynyl.
33b. The compound of any one of embodiments 1b-31b, wherein R^{3a} is substituted or unsubstituted carbocyclyl, substituted or unsubstituted heterocyclyl, substituted or unsubstituted aryl, or substituted or unsubstituted heteroaryl.
34b. The compound of any one of embodiments 1b-31b, wherein R^{3a} is substituted or unsubstituted C₁₋₆ alkyl.
35b. The compound of any one of embodiments 1b-31b, wherein R^{3a} is substituted alkyl.
36b. The compound of any one of embodiments 1b-31b, wherein R^{3a} is unsubstituted alkyl.
37b. The compound of any one of embodiments 1b-31b, wherein R^{3a} is methyl.
38b. The compound of any one of embodiments 1b-31b, wherein R^{3a} is hydrogen.
39b. The compound of any one of embodiments 1b-38b, wherein R^{4a} and R^{4b} is each independently hydrogen, halogen, —CN, —NO₂, substituted or unsubstituted alkyl, substituted or unsubstituted alkenyl, substituted or unsubstituted alkynyl, —OR^{E5}, —OC(═O)R^{E5}, —OS(═O)₂OR^{E5}, —N(R^{E5})₂, or —N(R^{E5})C(═O)R^{E5}, —N(R^{E5})S(═O)₂R^{E5}, —N(R^{E5})S(═O)₂OR^{E5}; wherein each instance of R^{E}5 is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, or two R^{E5} groups are taken with the intervening atoms to form a substituted or unsubstituted heterocyclic ring.
40b. The compound of any one of embodiments 1b-38b, wherein R^{4a} and R^{4b} is each independently hydrogen, halogen, —CN, —NO₂, —OR^{F6}, —OC(═O)R^{F6}, —N(R^{F6})₂, or —N(R^{F6})C(═O)R^{F6}; wherein each instance of R^{F6} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, or two R^{F6} groups are taken with the intervening atoms to form a substituted or unsubstituted heterocyclic ring.
41b. The compound of any one of embodiments 1b-38b, wherein R^{4a} and R^{4b} are independently hydrogen, —OH, or substituted or unsubstituted C₁₋₆ alkyl.
42b. The compound of any one of embodiments 1b-38b, wherein each of R^{4a} and R^{4b} are independently hydrogen, —OH, C₁₋₆ alkyl, C₁₋₆ haloalkyl, C₁₋₆ alkoxy, or C₁₋₆ haloalkoxy.
43b. The compound of any one of embodiments 1b-38b, wherein R^{4a} and R^{4b} are independently —CH₃, —CH₂CH₃, —OH, —OCH₃, or —CH(CH₃)₂.

44b. The compound of any one of embodiments 1b-38b, wherein R^{4a} and R^{4b} are both hydrogen.
45b. The compound of any one of embodiments 1b-38b, wherein R^{4a} and R^{4b} are joined to form an oxo (═O) group.
46b. The compound of any one of embodiments 1b-45b, wherein R⁵ is hydrogen in the cis position, relative to R¹⁹.
47b. The compound of any one of embodiments 1b-45b, wherein R⁵ is hydrogen in the trans position, relative to R¹⁹.
48b. The compound of any one of embodiments 1b-45b, wherein R⁵ is methyl in the cis position, relative to R¹⁹.
49b. The compound of any one of embodiments 1b-45b, wherein R⁵ is methyl in the trans position, relative to R¹⁹.
50b. The compound of any one of embodiments 1b-49b, wherein R^{6a} and R^{6b} is independently hydrogen, halogen, substituted or unsubstituted alkyl, substituted or unsubstituted alkenyl, or substituted or unsubstituted alkynyl.
51b. The compound of any one of embodiments 1b-49b, wherein R^{6a} and R^{6b} is independently hydrogen or substituted alkyl.
52b. The compound of any one of embodiments 1b-49b, wherein R^{6a} and R^{6b} is independently hydrogen or unsubstituted alkyl.
53b. The compound of any one of embodiments 1b-49b, wherein both R^{6a} and R^{6b} are hydrogen.
54b. The compound of any one of embodiments 1b-49b, wherein R^{6a} is halo or alkyl and R^{6b} is hydrogen.
55b. The compound of any one of embodiments 1b-49b, wherein R^{6a} and R^{6b} are both halo.
56b. The compound of any one of embodiments 1b-49b, wherein R^{6a} and R^{6b} are both alkyl.
57b. The compound of any one of embodiments 1b-56b, wherein R^{7a} and R^{7b} is each independently hydrogen, halogen, —CN, —NO₂, substituted or unsubstituted alkyl, substituted or unsubstituted alkenyl, substituted or unsubstituted alkynyl, —OR^{E}5, —OC(═O)R^{E}5, —OS(═O)₂OR^{E5}, —N(R^{E5})₂, or —N(R^{E5})C(═O)R^{E5}, —N(R^{E5})S(═O)₂R^{E5}, —N(R^{E5})S(═O)₂OR^{E5}; wherein each instance of R^{E5} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, or two R^{E}5 groups are taken with the intervening atoms to form a substituted or unsubstituted heterocyclic ring.
58b. The compound of any one of embodiments 1b-56b, wherein R^{7a} and R^{7b} is each independently hydrogen, halogen, —CN, —NO₂, —OR^{F6}, —OC(═O)R^{F6}, —N(R^{F6})₂, or —N(R^{F6})C(═O)R^{F6}; wherein each instance of R^{F6} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, or two R^{F6} groups are taken with the intervening atoms to form a substituted or unsubstituted heterocyclic ring.
59b. The compound of any one of embodiments 1b-56b, wherein R^{7a} and R^{7b} are independently hydrogen, —OH, or substituted or unsubstituted C₁₋₆ alkyl.
60b. The compound of any one of embodiments 1b-56b, wherein each of R^{7a} and R^{7b} are independently hydrogen, —OH, C₁₋₆ alkyl, C₁₋₆ haloalkyl, C₁₋₆ alkoxy, or C₁₋₆ alkoxyhalo.
61b. The compound of any one of embodiments 1b-56b, wherein R^{7a} and R^{7b} are independently —CH₃, —CH₂CH₃, —OH, —OCH₃, or —CH(CH₃)₂.
62b. The compound of any one of embodiments 1b-56b, wherein R^{7a} and R^{7b} are both hydrogen.
63b. The compound of any one of embodiments 1b-56b, wherein R^{7a} and R^{7b} are joined to form an oxo (═O) group.
64b. The compound of any one of embodiments 1b-63b, wherein R^{11a} and R^{11b} is each independently hydrogen, halogen, —CN, —NO₂, substituted or unsubstituted alkyl, substituted or unsubstituted alkenyl, substituted or unsubstituted alkynyl, —OR^{E}5, —OC(═O)R^{E}5, —OS(═O)₂OR^{E5}, —N(R^{E5})₂, or —N(R^{E5})C(═O)R^{E5}, —N(R^{E5})S(═O)₂R^{E5}, —N(R^{E5})S(═O)₂OR^{E5}; wherein each instance of R^{E5} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, or two R^{E5} groups are taken with the intervening atoms to form a substituted or unsubstituted heterocyclic ring.
65b. The compound of any one of embodiments 1b-63b, wherein R^{11a} and R^{1b} is each independently hydrogen, halogen, —CN, —NO₂, —OR^{F6}, —OC(═O)R^{F6}, —N(R^{F6})₂, or —N(R^{F6})C(═O)R^{F6}; wherein each instance of R^{F6} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, or two R^{F6} groups are taken with the intervening atoms to form a substituted or unsubstituted heterocyclic ring.
66b. The compound of any one of embodiments 1b-63b, wherein R^{11a} and R^{11b} are independently hydrogen, —OH, or substituted or unsubstituted C₁₋₆ alkyl.
67b. The compound of any one of embodiments 1-63, wherein each of R^{1a} and R^{11b} are independently hydrogen, —OH, C₁₋₆ alkyl, C₁₋₆ haloalkyl, C₁₋₆ alkoxy, or C₁₋₆ alkoxyhalo.
68b. The compound of any one of embodiments 1b-63b, wherein R^{11a} and R¹Ib are independently —CH₃, —CH₂CH₃, —OH, —OCH₃, or —CH(CH₃)₂.
69b. The compound of any one of embodiments 1b-63b, wherein R^{1a} and R¹Ib are both hydrogen.
70b. The compound of any one of embodiments 1b-63b, wherein R^{11a} and R^{11b} are joined to form an oxo (═O) group.
71b. The compound of any one of embodiments 1b-70b, wherein R^{12a} and R^{12b} is each independently hydrogen, halogen, —CN, —NO₂, substituted or unsubstituted alkyl, substituted or unsubstituted alkenyl, substituted or unsubstituted alkynyl, —OR^{E}5, —OC(═O)R^{E}5, —OS(═O)₂OR^{E5}, —N(R^{E5})₂, or —N(R^{E5})C(═O)R^{E5}, —N(R^{E5})S(═O)₂R^{E5}, —N(R^{E5})S(═O)₂OR^{E5}; wherein each instance of R^{E}5 is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, or two R^{E5} groups are taken with the intervening atoms to form a substituted or unsubstituted heterocyclic ring.
72b. The compound of any one of embodiments 1b-70b, wherein R^{2a} and R^{12b} is each independently hydrogen, halogen, —CN, —NO₂, —OR^{F6}, —OC(═O)R^{F6}, —N(R^{F6})₂, or —N(R^{F6})C(═O)R^{F6}; wherein each instance of R^{F6} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, or two R^{F6} groups are taken with the intervening atoms to form a substituted or unsubstituted heterocyclic ring.
73b. The compound of any one of embodiments 1b-70b, wherein R^{6a} and R^{2b} are independently hydrogen, —OH, or substituted or unsubstituted C₁₋₆ alkyl.
74b. The compound of any one of embodiments 1b-70b, wherein each of R² and R^{12b} are independently hydrogen, —OH, C₁₋₆ alkyl, C₁₋₆ haloalkyl, C₁₋₆ alkoxy, or C₁₋₆ alkoxyhalo.
75b. The compound of any one of embodiments 1b-70b, wherein R^{2a} and R^{12b} are independently —CH₃, —CH₂CH₃, —OH, —OCH₃, or —CH(CH₃)₂.

76b. The compound of any one of embodiments 1b-70b, wherein R^{6a} and R²⁶ are both hydrogen.
77b. The compound of any one of embodiments 1b-70b, wherein R¹² and R²⁶ are joined to form an oxo (═O) group.
78b. The compound of any one of embodiments 1b-77b, wherein R¹?b is fluorine, hydroxyl, methyl, or hydrogen.
79b. The compound of any one of embodiments 1b-77b, wherein R¹?b is hydrogen.
80b. The compound of any one of embodiments 1b-79b, wherein R¹⁹ is substituted or unsubstituted C₃₋₆ carbocyclyl, or substituted or unsubstituted C₆₋₁₀ aryl.
81b. The compound of any one of embodiments 1b-79b, wherein R¹⁹ is substituted or unsubstituted C₃₋₆ carbocyclyl.
82b. The compound of any one of embodiments 1b-79b, wherein R¹⁹ is substituted or unsubstituted C₆₋₁₀ aryl.
83b. The compound of any one of embodiments 1b-79b, wherein R¹⁹ is selected from the group consisting of:

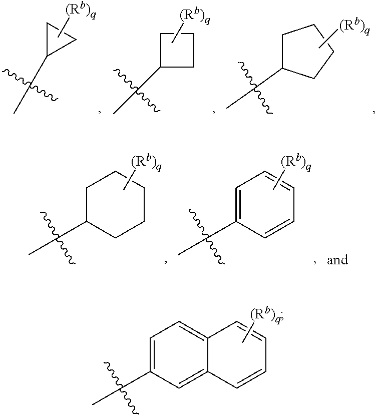

wherein:
   each instance of R^{b} is, independently hydrogen, halogen, —NO₂, —CN, —OR^{G7}, —N(R^{G7})₂, —C(═O)R^{G7}, —C(═O)OR^{G7}, —C(═O)N(R^{G7})₂, —OC(═O)R^{G7}, —OC(═O)OR^{G7}, —N(R^{G7})C(═O)R^{G7}, —OC(═O)N(R^{G7})₂, —N(R^{G7})C(═O)OR^{G7}, —S(═O)₂R^{G7}, —S(═O)₂OR^{G7}, —OS(═O)₂R^{G7}, —S(═O)₂N(R^{G7})₂, or —N(R^{G7})S(═O)₂R^{G7}, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl;
   each instance of R^{G7} is independently hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ carbocylyl, substituted or unsubstituted 3- to 6-membered heterocylyl, substituted or unsubstituted C₅₋₁₀ aryl, substituted or unsubstituted 5- to 10-membered heteroaryl, an oxygen protecting group when attached to oxygen, a nitrogen protecting group when attached to nitrogen, or two R^{G7} groups are taken with the intervening atoms to form a substituted or unsubstituted heterocyclic ring; and
   q is an integer selected from 0 to 11.
84b. The compound of any one of embodiments 1b-83b, wherein n is 0.
85b. The compound of any one of embodiments 1b-83b, wherein n is 1.
86b. The compound of any one of embodiments 1b-85b, wherein R^{1a} is methyl.
87b. The compound of any one of embodiments 1b-85b, wherein R^{1a} is ethyl.
88b. The compound of any one of embodiments 1b-85b, wherein R¹⁸ is substituted methyl.
89b. The compound of any one of embodiments 1b-85b, wherein R¹⁸ is substituted ethyl.
90b. The compound of any one of embodiments 1b-89b, wherein t is 2.
91b. The compound of any one of embodiments 1b-89b, wherein t is 3.
92b. The compound of any one of embodiments 1b-91b, wherein r is 2.
93b. The compound of any one of embodiments 1b-91b, wherein r is 3.
94b. The compound of any one of embodiments 1b-93b, wherein n is 2.
95b. The compound of embodiment 3b, wherein the compound of Formula III is a compound of Formula III-ad:

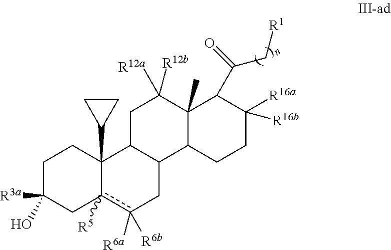

or a pharmaceutically acceptable salt thereof.
96b. The compound of embodiment 3b, wherein the compound of Formula III is a compound of Formula III-bd:

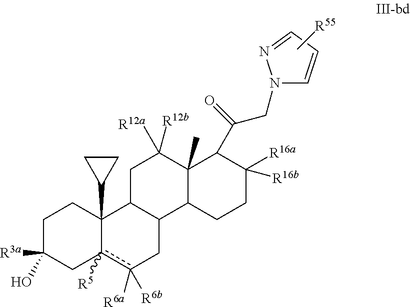

or a pharmaceutically acceptable salt thereof,
wherein R⁵⁵ is hydrogen, halogen, cyano, or substituted or unsubstituted alkyl.
97b. A pharmaceutical composition comprising a compound of any one of embodiments 1b-96b or a pharmaceutically acceptable salt thereof, and a pharmaceutically acceptable excipient.
98b. A method of treating a CNS-related disorder in a subject in need thereof, comprising administering to the subject an effective amount of a compound of any one of embodiments 1b-96b or a pharmaceutically acceptable salt thereof.
99b. The method of embodiment 98b, wherein the CNS-related disorder is a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, or status epilepticus.
100b. The method of embodiment 98b, wherein the CNS-related disorder is depression.
101b. The method of embodiment 98b, wherein the CNS-related disorder is postpartum depression.
102b. The method of embodiment 98b, wherein the CNS-related disorder is major depressive disorder.
103b. The method of embodiment 102b, wherein the major depressive disorder is moderate major depressive disorder.
104b. The method of embodiment 102b, wherein the major depressive disorder is severe major depressive disorder.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Materials and Methods

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, HPLC, or supercritical fluid chromatography (SFC). The following schemes are presented with details as to the preparation of representative oxysterols that have been listed herein. The compounds provided herein may be prepared from known or commercially available starting materials and reagents by one skilled in the art of organic synthesis. Exemplary chiral columns available for use in the separation/purification of the enantiomers/diastereomers provided herein include, but are not limited to, CHIRALPAK® AD-10, CHIRALCEL® OB, CHIRALCEL® OB—H, CHIRALCEL® OD, CHIRALCEL® OD-H, CHIRALCEL® OF, CHIRALCEL® OG, CHIRALCEL® OJ and CHIRALCEL® OK.

¹H-NMR reported herein (e.g., for the region between 8 (ppm) of about 0.5 to about 4 ppm) will be understood to be an exemplary interpretation of the NMR spectrum (e.g., exemplary peak integratations) of a compound.

LC-ELSD/MS: (Mobile Phase: 1.5 ML/4L TFA in water (solvent A) and 0.75 ML/4L TFA in acetonitrile (solvent B), using the elution gradient 30%-90% (solvent B) over 0.9 minutes and holding at 90% for 0.6 minutes at a flow rate of 1.2 ml/min; Column: Xtimate C18 2.1*30 mm, 3 um; Wavelength: UV 220 nm; Column temperature: 50° C.; MS ionization: ESI; Detector: PDA & ELSD.

### Abbreviations:

LDA: Lithium diisopropylamide; DMP: Dess-Martin periodinane; DVIE: 1,2-dimethoxyethane; Na₂SO₄: sodium sulfate; PE: petroleum ether; EtOAc: ethylacetate; t-BuOK: potassium 2-methylpropan-2-olate. Me: methyl; t-Bu: tert-butyl; THF: tetrahydrofuran; DCM: dichloromethane; Ph: Phenyl; HATU: 2-(7-Aza-1H-benzotriazole-1-yl)-1,1,3,3-tetramethyluronium hexafluorophosphate; MAD: methyl aluminum bis(2,6-di-t-butyl-4-methylphenoxide).

### Example 1 & 2: Synthesis of 1-((1S,4aS,4bR,6aR,9S,11aS,11bR,13aS)-9-hydroxy-9,13a-dimethyloctadecahydro-1H-cyclohepta[a]phenanthren-1-yl)ethan-1-one & 1-((4aS,4bR,6aR,9S,11aS,11bR,13aS)-9-hydroxy-9,13a-dimethyloctadecahydro-1H-cyclohepta[a]phenanthren-1-yl)ethan-1-one

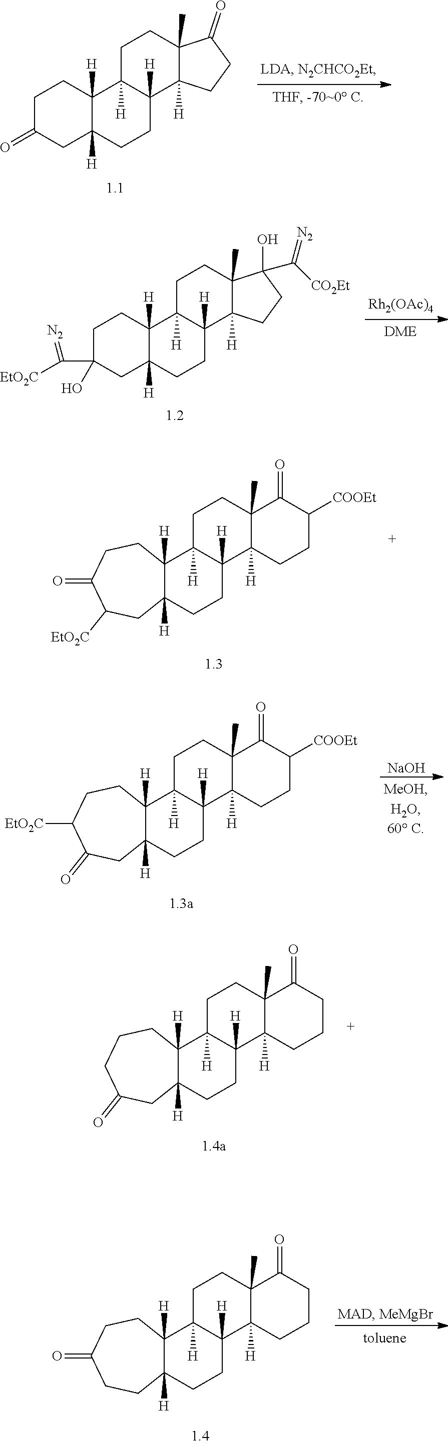

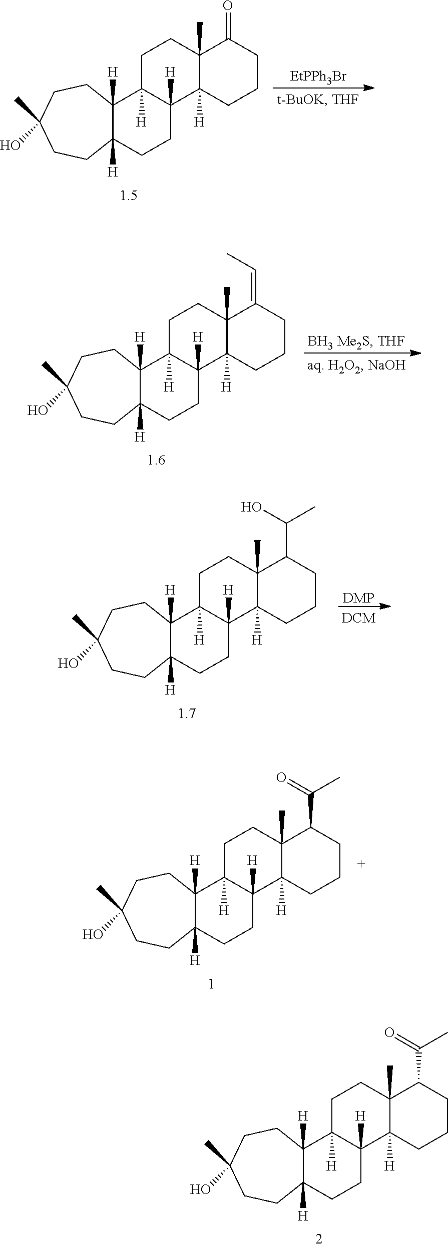

### Synthesis of 1.2

To a cold (−78° C.) LDA solution (145 mmol, 1 M in THE) was added a solution of 1.1 (5.0 g, 18.2 mmol) and ethyl diazoacetate (16.5 g, 145 mmol) in THE (150 mL). After stirring at −70° C. for 2 h, the reaction was quenched with acetic acid (8.70 g, 145 mmol) in THF (50 mL). After warming to rt over 16 h, the reaction mixture was diluted with water (300 mL) and extracted with EtOAc (3×150 mL). The combined organic layers were washed with saturated brine (2×200 mL), dried over anhydrous Na₂SO₄, filtered and concentrated to give 1.2 (20 g) as an oil, which was used directly in next step.

### Synthesis of 1.3 & 1.3a

To a solution of 1.2 (20 g, 39.7 mmol) in DME (150 mL) was added Rh₂(OAc)₄ (350 mg, 0.794 mmol). After stirring at 25° C. for 16 h, the reaction mixture was concentrated. The residue was purified by silica gel chromatography (0-20% of EtOAc in PE) to give a mixture of diastereomers 1.3 & 1.3a (11 g) as an oil.

### Synthesis of 1.4 & 1.4a

To a mixture of 1.3 & 1.3A (11 g, 24.6 mmol) in MeOH (100 mL) was added H₂O (50 mL) and NaOH (7.84 g, 196 mmol). After stirring at 60° C. for 16 h, the reaction was diluted with H₂O (200 mL) and extracted with EtOAc (2×200 mL). The combined organic phase was washed with saturated brine (200 mL), dried over anhydrous Na₂SO₄, filtered and concentrated. The residue was purified by flash column (0-30% of EtOAc in PE) to give 1.4 & 1.4a (2.6 g, 34.9%) as a solid. The diastereomers were separated by SFC (column: DAICEL CHIRALCEL OJ (250 mm*50 mm, 10 um); Mobile phase: A: CO₂ B: 0.1% NH₃H₂O EtOH; gradient: from 20% to 20% of B, FlowRate (ml/min): 180) to give 1.4a (1 g) and 1.4 (1 g) both as solids.

¹H NMR (400 MHz, CDCl₃) δ ppm 3.11-2.86 (m, 1H), 2.70-2.55 (m, 1H), 2.53-2.31 (m, 2H), 2.26-2.15 (m, 1H), 2.10-1.91 (m, 4H), 1.85-1.48 (m, 12H), 1.43-1.17 (m, 5H), 1.10 (s, 3H), 0.92-0.84 (m, 1H); LC-ELSD/MS purity 99%, anlytic SFC: 100% de; MS ESI calcd. for C₂₀H₃₀O₂ [M+H]⁺ 303.2, found 303.2.

¹H NMR (400 MHz, CDCl₃) δ ppm 2.68-2.38 (m, 4H), 2.36-2.25 (m, 1H), 2.24-2.14 (m, 1H), 2.11-1.58 (m, 13H), 1.56-1.13 (m, 6H), 1.10 (s, 3H), 1.09-0.85 (m, 2H); LC-ELSD/MS purity 99%, anlytic SFC: 100% de; MS ESI calcd. for C₂₀H₃₀O₂[M−H₂O+H]⁺ 285.2, found 285.2.

### Synthesis of 1.5

To the fresh prepared MAD (32.6 mmol) solution in toluene (10 mL) (see 15.4) was added 1.4 (3.3 g, 10.9 mmol) in DCM (30 mL) dropwise at −70° C. After stirring at −70° C. for 1 h under N₂, CH₃BrMg (14.5 mL, 43.6 mmol, 3M in ethyl ether) was added dropwise at −70° C. After stirring at −70° C. for another 4 h, the reaction mixture was poured into citric acid (50 mL, 20%) below 10° C. and extracted with EtOAc (3×100 mL). The combined organic layer was washed brine (2×50 mL), dried over Na₂SO₄, filtered and concentrated in vacuum. The residue was purified by flash column (0˜30% of EtOAc in PE) to give 1.5 (710 mg).
1.5: ¹HNMR (400 MHz, CDCl3) δ ppm 2.68-2.54 (m, 1H), 2.27-2.14 (m, 1H), 2.11-1.97 (m, 1H), 1.89-1.56 (m, 11H), 1.54-1.29 (m, 7H), 1.21 (s, 3H), 1.19-1.11 (m, 2H), 1.09 (s, 3H), 1.07-0.76 (m, 4H); LC-ELSD/MS purity 99%, MS ESI calcd. for C₂₁H₃₄O₂[M−H₂O+H]⁻ 301.3, found 301.3.

### Synthesis of 1.6

To a mixture of MePPh₃Br (4.71 g, 12.7 mmol) in THE (15 mL) was added t-BuOK (1.42 g, 12.7 mmol) at 15° C. under N₂. After stirring at 60° C. for 30 min, 1.5 (680 mg, 2.13 mmol) was added in portions below 60° C. After stirring at 60° C. for 16 h, the reaction mixture was quenched with 10% NH₄Cl aqueous (100 mL) at 15° C. and extracted with EtOAc (3×50 mL). The combined organic layer was washed brine (2×50 mL), dried over Na₂SO₄, filtered and concentrated in vacuum. The residue was purified by flash column (0˜30% of EtOAc in PE) to give 1.6 (780 mg) as a solid. 1HNMR (400 MHz, CDCl3) δ_{H} 5.22-5.08 (m, 1H), 2.59-2.43 (m, 1H), 2.30-2.07 (m, 1H), 2.02-1.58 (m, 14H), 1.56-1.28 (m, 7H), 1.21 (s, 3H), 1.16-1.03 (m, 5H), 0.93 (s, 4H), 0.89-0.69 (m, 2H).

### Synthesis of 1.7

To a solution of 1.6 (780 mg, 2.35 mmol) in THE (20 mL) was added BH₃·Me₂S (714 mg, 940 μL, 9.40 mmol, 10 M). After stirring at 25° C. for 16 h, the reaction mixture was diluted with ethanol (2.35 mL) at 15° C., NaOH aqueous (4.70 mL, 5.0 M, 23.5 mmol) at 0° C. and finally hydrogen peroxide (2.35 mL, 10 M, 23.5 mmol) dropwise at 0° C. After stirring at 70° C. for 1 h, the reaction mixture was diluted with Na₂S₂O₃ (100 mL, sat.aq.) and extracted with EtOAc (3×100 mL). The combined organic layer was washed brine (2×50 mL), dried over Na₂SO₄, filtered and concentrated in vacuum to give 1.7 (1.1 g) as a solid. ¹HNMR (400 MHz, CDCl3) δ_{H} 4.43-3.97 (m, 1H), 1.95-1.31 (m, 25H), 1.19-1.03 (m, 6H), 1.02-0.81 (m, 9H), 0.78-0.75 (m, 2H).

### Synthesis of 1 & 2

To a solution of 1.7 (1 g, 2.86 mmol) in DCM (30 mL) was added Dess-martin (2.42 g, 5.72 mmol) at 40° C. After stirring at 40° C. for 5 min, the reaction mixture was quenched with saturated NaHCO₃ aqueous (100 mL). The DCM phase was separated and washed with saturated NaHCO₃/Na₂S₂O₃ aqueous (1:1, 2×100 mL), brine (2×50 mL), dried over Na₂SO₄, filtered and concentrated under vacuum to give 1 (19.5 mg, 1.96%) and a mixture of keto diastereomers 2 (500 mg) both as solids.

1: ¹HNMR (400 MHz, CDCl3)⁶H 2.30 (dd, J=3.2, 12.8 Hz, 1H), 2.14 (s, 3H), 1.87-1.58 (m, 12H), 1.51-1.28 (m, 7H), 1.22 (s, 3H), 1.17-0.95 (m, 6H), 0.93 (s, 3H), 0.91-0.69 (m, 3H).

LC-ELSD/NS purity 99%, MS ESI calcd. for C₂₃H₃₈O₂[M−H₂O+H]⁺329.3, found 329.3.

2: ¹HNMR (400 MHz, CDCl3) S_{H} 2.49-2.26 (m, 1H), 2.17-2.09 (m, 3H), 1.93-1.58 (m, 10H), 1.58-1.27 (m, 12H), 1.23-1.19 (m, 3H), 1.17-0.96 (m, 4H), 0.94-0.92 (m, 3H), 0.88-0.75 (m, 2H).

### Example 3 & 4: Synthesis of 1-(2-((1R,4aS,4bR,6aR,9S,11aS,11bR,13aS)-9-hydroxy-9,13a-dimethyloctadecahydro-1H-cyclohepta[alphenanthren-1-yl)-2-oxoethyl)-1H-pyrazole-4-carbonitrile & 1-(2-((1S,4aS,4bR,6aR,9S,11aS,11bR,13aS)-9-hydroxy-9,13a-dimethyloctadecahydro-1H-cyclohepta[a]phenanthren-1-yl)-2-oxoethyl)-1H-pyrazole-4-carbonitrile

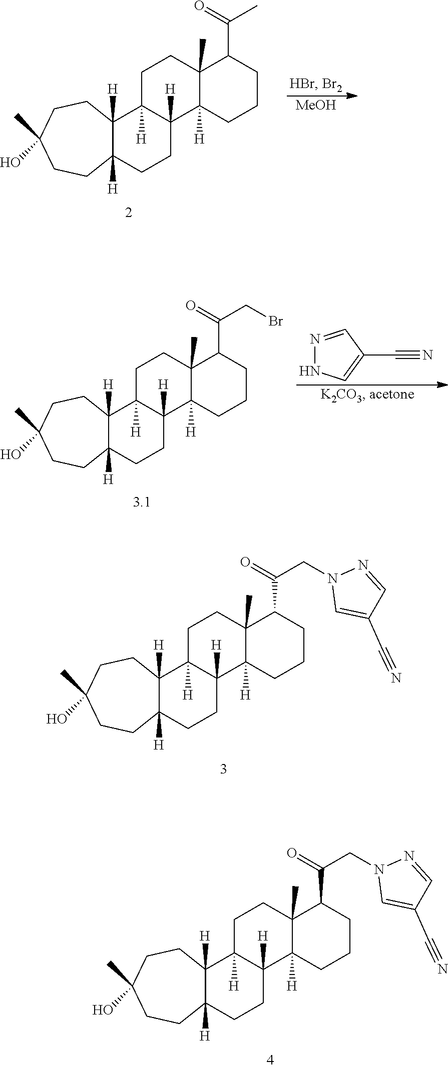

### Synthesis of 3.1

To a solution of 2 (130 mg, 0.3751 mmol) in MeOH (10 ml) was added HBr (15.1 mg, 0.07502 mmol, 40% in water) and Br₂ (71.9 mg, 0.4501 mmol). After stirring at 25° C. for 2 h, the reaction mixture was quenched by sat. aq NaHCO₃ (10 mL), diluted with water (20 mL), and extracted with EtOAc (2×30 mL). The combined organic phase was washed with brine (30 mL), dried over anhydrous Na₂SO₄, filtered, concentrated in vacuum to afford 3.1 (160 mg) as a solid which used directly for the next step.

¹HNMR (400 MHz, CDCl3) δ_{H} 4.01-3.85 (m, 2H), 2.75-2.53 (m, 1H), 1.94-1.56 (m, 13H), 1.54-1.29 (m, 10H), 1.23-1.16 (m, 4H), 1.15-0.99 (m, 5H), 0.98-0.94 (m, 4H), 0.91-0.68 (m, 3H).

### Synthesis of 3 & 4

To a solution of 3.1 (160 mg, 0.3760 mmol) in acetone (5 mL) was added 4-cyanopyrazole (42.0 mg, 0.4512 mmol) and K₂CO₃ (52.7 mg, 0.376 mmol). After stirring at 20° C. for 16 h, the reaction mixture was diluted with water (50 mL) and extracted with EtOAc (3×50 mL). The organic layer was separated and concentrated. The residue was purified by flash column (0˜50% of EtOAc in PE) to give 3 (30 mg) and 4 (42.7 mg, 26.0% yield) as solids.

3: ¹H NMR (400 MHz, CDCl₃) δ_{H} 7.87 (s, 1H), 7.83 (s, 1H), 5.15-4.76 (m, 2H), 2.46 (d, J=4.8 Hz, 1H), 1.97-1.58 (m, 11H), 1.56-1.30 (m, 11H), 1.20 (s, 3H), 1.17-1.03 (m, 3H), 1.00 (s, 3H), 0.97-0.75 (m, 3H); LC-ELSD/MIS purity 99%, MS ESI calcd. for C₂₇H₃₉N₃O₂ [M−H₂O+H]⁺ 420.3, found 420.3.

4: ¹H NMR (400 MHz, CDCl₃) δ_{H} 7.87 (s, 1H), 7.81 (s, 1H), 5.11-4.89 (m, 2H), 2.32 (dd, J=3.2, 12.4 Hz, 1H), 1.91-1.56 (m, 12H), 1.54-1.25 (m, 8H), 1.22 (s, 3H), 1.21-0.97 (m, 6H), 0.96 (s, 3H), 0.92-0.75 (m, 2H); LC-ELSD/MS purity 99%, MS ESI calcd. for C₂₇H₃₉N₃O₂ [M−H₂O+H]⁺420.3, found 420.3.

### Example 9: Synthesis of 1-((1S,4aS,4bR,6aR,8R,11aS,11bR,13aS)-8-hydroxy-8,13a-dimethyloctadecahydro-1H-cyclohepta[a]phenanthren-1-yl)ethan-1-one

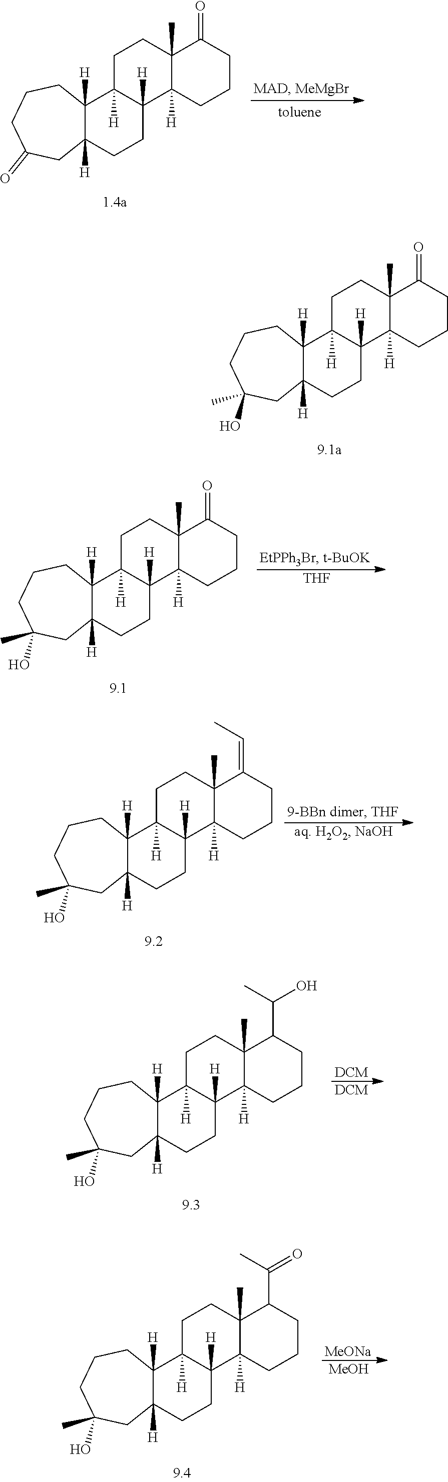

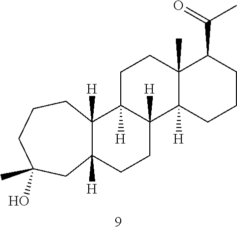

### Synthesis of 9.1 & 9.1a

To the fresh prepared MAD (14.8 mmol, in 20 mL toluene) solution (see 15.4) was added 1.4a (1.5 g, 4.95 mmol) in DCM (20 mL) dropwise at −70° C. After stirring at −70° C. for 1 h under N₂, CH₃BrMg (6.60 mL, 19.8 mmol, 3M in ethyl ether) was added dropwise at −70° C. After stirring at −70° C. for 4 h, the reaction mixture was poured into citric acid (50 mL, 20%) at 10° C. and extracted with EtOAc (2×50 mL). The combined organic layer was dried over Na₂SO₄, filtered and concentrated in vacuum. The residue was purified by silica gel chromatography (0-25% of EtOAc in PE) to give the 9.1 (900 mg, 57.3%) and 9.1a (0.4 g, 25.4%) both as solids. [05981 9.1: ¹H NMR (400 MHz, CDCl₃) S_{H} 2.66-2.56 (m, 1H), 2.23-2.15 (m, 1H), 2.10-2.00 (m, 1H), 1.97-1.69 (m, 7H), 1.63-1.57 (m, 3H), 1.55-1.29 (m, 7H), 1.27 (s, 3H), 1.25-1.11 (m, 5H), 1.09 (s, 3H), 1.06-0.80 (m, 3H); LC-ELSD/MS purity>99%, 100% de based on H-NMR; MS ESI calcd. for C₂₁H₃₄O₂[M+H-H₂O]⁺ 301.2, found 301.2.

9.1a: ¹H NMR (400 MHz, CDCl₃) δ_{H} 2.68-2.55 (m, 1H), 2.48-2.32 (m, 1H), 2.25-2.14 (m, 1H), 2.11-1.98 (m, 2H), 1.8-1.59 (m, 8H), 1.5-1.33 (m, 7H), 1.30-1.23 (m, 1H), 1.21 (s, 3H), 1.19-1.12 (m, 2H), 1.09 (s, 3H), 1.06-0.72 (m, 5H).

### Synthesis of 9.2

To a solution of EtPPh₃Br (5.56 g, 15.0 mmol) in THE (10 mL) was added t-BuOK (1.68 g, 15.0 mmol) at 15° C. After stirring at 50° C. for 1 h, solution of 9.1 (800 mg, 2.5 mmol) in THE (10 mL) was added. After stirring at 60° C. for 16 h, the reaction mixture was added into saturated NH₄Cl (100 mL) and extracted with EtOAc (3×50 mL). The combined organic layer was washed with saturated brine (100 mL), dried over anhydrous Na₂SO₄, filtered and concentrated. The residue was purified by flash column (0-8% of EtOAc in PE) to give 9.2 (640 mg, 77%) as an oil. ¹H NMR (400 MHz, CDCl₃) δ_{H} 5.22-5.08 (m, 1H), 2.55-1.59 (m, 14H), 1.52-1.28 (m, 6H), 1.26 (s, 3H), 1.25-0.75 (m, 14H).

### Synthesis of 9.3

To a solution of 9.2 (640 mg, 1.9 mmol) in THE (10 mL) was added BH₃Me₂S (1 mL, 10 M, 10.0 mmol) at 20° C. After stirring at 20° C. for 16 h, the reaction mixture was diluted with EtOH (2.66 g, 57.9 mmol), NaOH (11.5 mL, 5M, 57.9 mmol) at 0° C., and finally by H₂O₂(6.53 g, 57.9 mmol, 30%) dropwise. After stirring at 75° C. for 1 h, the reaction was diluted with saturated brine (50 mL) and extracted with EtOAc (3×20 mL). The combined organic layer was washed with saturated Na₂S₂O₃ (100 mL), saturated brine (100 mL), dried over anhydrous Na₂SO₄, filtered and concentrated to give 9.3 (600 mg) as a solid.

### Synthesis of 9.4

To a solution of 9.3 (600 mg, 1.7 mmol) DCM (20 mL) was added DMP (1.45 g, 3.4 mmol). After stirring at 40° C. for 30 min, the mixture was added into saturated NaHCO₃ (100 mL). and extracted with DCM (3×30 mL). The combined organic layer was washed with saturated Na₂S₂O₃ (2×100 mL), saturated brine (100 mL), dried over anhydrous Na₂SO₄, filtered and concentrated to give 9.4 (800 mg) as a solid. ¹H NMR (400 MHz, CDCl₃) δ_{H} 2.55-2.29 (m, 1H), 2.18-2.12 (m, 3H), 2.00-1.59 (m, 13H), 1.52-1.28 (m, 7H), 1.27-1.24 (m, 3H), 1.20-0.95 (m, 5H), 0.93-0.92 (m, 3H), 0.90-0.75 (m, 3H).

### Synthesis of 9

To a solution of 9.4 (800 mg, 2.3 mmol) in MeOH (40 mL) was added MeONa (2.23 g, 41.4 mmol). After stirring at 80° C. for 40 h, the reaction mixture was added saturated NH₄Cl (100 mL) and extracted with DCM (3×30 mL). The combined organic layer was washed with saturated brine (100 mL), dried over anhydrous Na₂SO₄, filtered and concentrated. The residue was purified by flash column (0-20% of EtOAc in PE) to give 9 (275 mg, 35%) as a solid. ¹H NMR (400 MHz, CDCl₃) δ_{H} 2.29 (dd, J=3.2 Hz, 12.8 Hz, 1H), 2.14 (s, 3H), 2.00-1.59 (m, 10H), 1.52-1.28 (m, 7H), 1.27 (s, 3H), 1.25-0.95 (m, 8H), 0.93 (s, 3H), 0.90-0.75 (m, 3H). LC-ELSD/MS purity 99%, MS ESI calcd. for C₂₃H₃₇O [M−H₂O+H]⁻ 329.3, found 329.3.

### Example 10: Synthesis of 1-(2-((1S,4aS,4bR,6aR,8R,11aS,11bR,13aS)-8-hydroxy-8,13a-dimethyloctadecahydro-1H-cyclohepta[alphenanthren-1-yl)-2-oxoethyl)-1H-pyrazole-4-carbonitrile

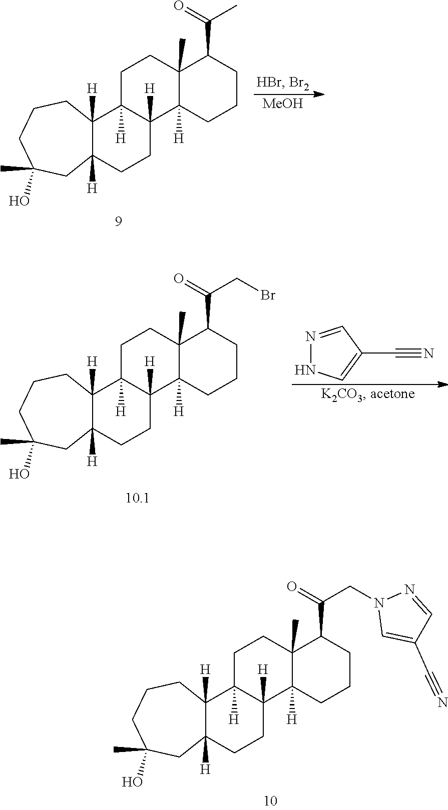

### Synthesis of 10.1

To a solution of 9 (240 mg, 0.69 mmol) and HBr (27.4 mg, 0.14 mmol, 40%) in MeOH (10 mL) was added Br₂ (116 mg, 0.73 mmol) at 0° C. After stirring at 20° C. for 2 h, the mixture was added into saturated NaHCO₃ (50 mL) and extracted with EtOAc (3×20 mL). The combined organic layer was washed with saturated brine (50 mL), dried over anhydrous Na₂SO₄, filtered and concentrated to give 10.1 (300 mg) as an oil. ¹H NMR (400 MHz, CDCl₃) S_{H} 4.00-3.80 (m, 2H), 2.57 (dd, J=3.2 Hz, 12.8 Hz, 1H), 2.00-1.59 (m, 11H), 1.52-1.28 (m, 7H), 1.27 (s, 3H), 1.25-0.98 (m, 7H), 0.95 (s, 3H), 0.94-0.75 (m, 3H).

### Synthesis of 10

To a solution of 10.1 (150 mg, 0.35 mmol) in acetone (5 mL) were added K₂CO₃ (97.2 mg, 0.71 mmol) and 1H-pyrazole-4-cabonitrile (49.2 mg, 0.53 mmol). After stirring at 20° C. for 2 h, the reaction mixture was added into saturated NH₄Cl (50 mL) and extracted with EtOAc (3×20 mL). The combined organic layer was washed with water (2×100 mL), saturated brine (100 mL), dried over anhydrous Na₂SO₄, filtered and concentrated. The residue was purified by flash column (0-50% of EtOAc in PE) to give 10 (69.2 mg, 45%) as a solid. ¹H NMR (400 MHz, CDCl₃) δ_{H} 7.82 (s, 1H), 7.81 (s, 1H), 5.10-4.90 (m, 2H), 2.32 (dd, J=3.2 Hz, 12.8 Hz, 1H), 2.00-1.59 (m, 11H), 1.52-1.28 (m, 7H), 1.27 (s, 3H), 1.25-0.98 (m, 7H), 0.96 (s, 3H), 0.95-0.75 (m, 3H). LC-ELSD/MS purity 99%, MS ESI calcd. for C₂₇H₃₈N₃O [M−H₂O+H]⁺420.3, found 420.3.

### Example 11 & 12: Synthesis of 1-((1S,4aS,4bR,6aR,8R,11aS,11bR,13aS)-8-hydroxy-8,13a-dimethyloctadecahydro-1H-cyclohepta[a]phenanthren-1-yl)-2-(5-methyl-2H-tetrazol-2-yl)ethan-1-one & 1-((1S,4aS,4bR,6aR,8R,11aS,11bR,13aS)-8-hydroxy-8,13a-dimethyloctadecahydro-1H-cyclohepta[a]phenanthren-1-yl)-2-(5-methyl-1H-tetrazol-1-yl)ethan-1-one

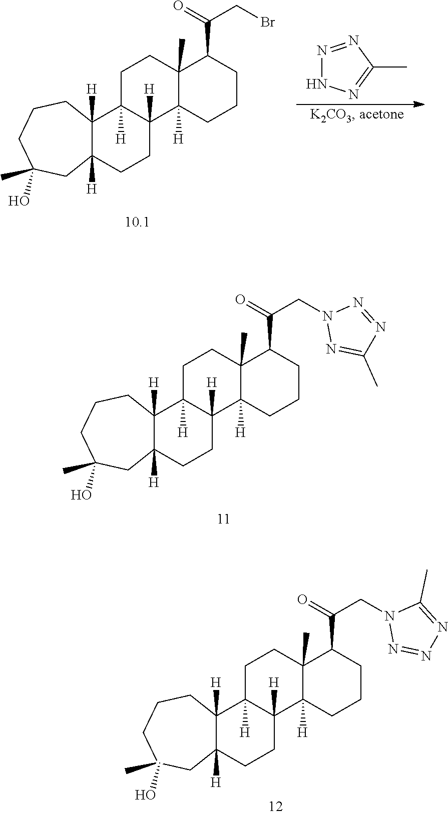

To a solution of 10.1 (150 mg, 0.35 mmol) in acetone were added K₂CO₃ (97.2 mg, 0.71 mmol) and 5-methyl-2H-1,2,3,4-tetrazole (44.4 mg, 0.53 mmol). After stirring at 20° C. for 2 h, the mixture was added into saturated NH₄Cl (50 mL) and extracted with EtOAc (3×20 mL). The combined organic layer was washed with saturated brine (100 mL), dried over anhydrous Na₂SO₄, filtered and concentrated. The residue was purified by flash column (0-100% of EtOAc in PE) to give 11 (20 mg) and 12 (24.2 mg, 16%) both as solids.

11 (20 mg) was further purified by pre-HPLC (Column: Welch Xtimate C18 150*25 mm*5 m, Condition: water (0.225% FA)-ACN, Begin B: 35, End B: 95, Gradient Time (min): 8.5, 100% B Hold Time (min): 2) to give 11 (4.6 mg, 23%) as a solid.

11: ¹H NMR (400 MHz, CDCl₃) δ_{H} 5.39 (s, 2H), 2.56 (s, 3H), 2.34 (dd, J=3.2 Hz, 12.8 Hz, 1H), 2.00-1.59 (m, 10H), 1.52-1.30 (m, 6H), 1.27 (s, 3H), 1.25-1.00 (m, 8H), 0.97 (s, 3H), 0.95-0.75 (m, 4H). LC/MS purity 99%, MS ESI calcd. for C₂₅H₃₉N₄O [M−H₂O+H]⁻ 411.4, found 411.4.

12: ¹H NMR (400 MHz, CDCl₃) δ_{H} 5.25-5.09 (m, 2H), 2.45 (s, 3H), 2.40 (dd, J=3.2 Hz, 12.8 Hz, 1H), 2.00-1.59 (m, 11H), 1.52-1.30 (m, 7H), 1.28 (s, 3H), 1.25-1.00 (m, 7H), 0.97 (s, 3H), 0.95-0.75 (m, 3H); LC-ELSD/MS purity 99%, MS ESI calcd. for C₂₅H₃₉N₄O [M−H₂O+H]⁺ 411.3, found 411.3.

### Example 13: Synthesis of 1-((1R,4aS,4bR,6aR,8R,11aS,11bR,13aS)-8-ethyl-8-hydroxy-13a-methyloctadecahydro-1H-cyclohepta[a]phenanthren-1-yl)ethan-1-one & 1-((1S,4aS,4bR,6aR,8R,11aS,11bR,13aS)-8-ethyl-8-hydroxy-13a-methyloctadecahydro-1H-cyclohepta[a]phenanthren-1-yl)ethan-1-one

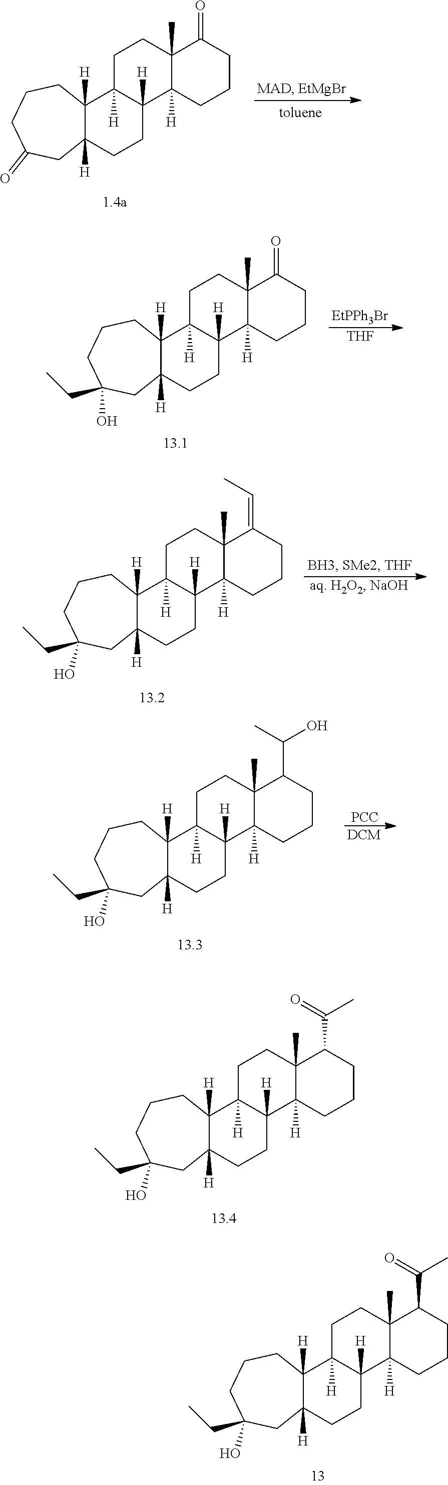

### Synthesis of 13.1

To the fresh prepared MAD (14.8 mmol, in 15 mL toluene) (see 15.4) was added 1.4a (1.5 g, 4.95 mmol) in DCM (15 mL) dropwise at −70° C. After stirring at −70° C. for 1 h under N₂, EtMgBr (6.60 mL, 19.8 mmol, 3M in ethyl ether) was added drop wise at −70° C. After stirring at −70° C. for another 4 h, the reaction mixture was poured into citric acid (50 mL, 20%) at 10° C. and extracted with EtOAc (2×50 mL). The combined organic layer was dried over Na₂SO₄, filtered and concentrated in vacuum. The residue was purified by silica gel chromatography (0-25% of EtOAc in PE) to give the 13.1 (630 mg, 38.4%) as a solid.

13.1: ¹H NMR (400 MHz, CDCl₃) δ_{H} 2.66-2.65 (m, 1H), 2.24-2.15 (m, 1H), 2.09-2.00 (m, 1H), 1.89-1.67 (m, 7H), 1.67-1.55 (m, 4H), 1.54-1.17 (m, 12H), 1.12 (s, 1H), 1.09 (s, 3H), 1.04-0.94 (m, 2H), 0.90 (t, J=7.6 Hz, 3H), 0.88-0.74 (m, 1H); LC-ELSD/MS purity 99%, MS ESI calcd. for C₂₂H₃₅O [M+H-H₂O]⁺315.3, found 315.3.

### Synthesis of 13.2

To a mixture of EtPPh3Br (4.19 g, 11.3 mmol) in THE (11 mL) was added t-BuOK (1.26 g, 11 3 mmol) at 15° C. under N₂. After stirring at 40° C. for 1 h, 13.1 (630 mg, 1.89 mmol) was added in portions below 50° C. After stirring at 65° C. for 16 h, the reaction mixture was quenched with 10% NH₄Cl aqueous (50 mL) at 15° C. and extracted with EtOAc (2×50 mL). The combined organic phase was concentrated under vacuum to give a white solid, which was purified by flash column (0-10% of EtOAc in PE) to give 13.2 (380 mg, 58.3%) as a solid.

31 ¹H NMR (400 MHz, CDCl₃)⁶H 5.1-5.11 (m, 1H), 2.54-2.46 (m, 1H), 2.27-2.07 (m, 1H), 1.96-1.68 (m, 10H), 1.61-1.56 (m, 9H), 1.51-1.30 (m, 7H), 1.19-0.99 (m, 5H), 0.94-0.88 (m, 6H).

### Synthesis of 13.3

To a solution of 13.2 (380 mg, 1.1 mmol) in THE (5 mL) was added BH₃·Me₂S (990 μL, 10 M, 9.90 mmol). After stirring at 15° C. for 16 h, the reaction mixture was diluted with EtOH (3.16 mL, 55.0 mmol), NaOH (2.2 g in 11 mL water, 5 M, 55.0 mmol) dropwise and H₂O₂(5.5 mL, 10 M, 55.0 mmol) dropwise at 0° C. After stirring at 78° C. for 2 h, the mixture was quenched by Na₂S₂SO₃ (30 mL, 10%) and extracted with EtOAc (2×20 mL). The combined organic layer was dried over Na₂SO₄, filtered and concentrated in vacuum to give 13.3 (272 mg) as a solid. ¹H NMR (400 MHz, CDCl₃) S_{H} 4.34-4.03 (m, 1H), 1.93-1.53 (m, 13H), 1.53-1.37 (m, 5H), 1.36-1.20 (m, 7H), 1.18-1.02 (m, 5H), 1.01-0.98 (m, 2H), 0.97-0.87 (m, 6H), 0.86-0.81 (m, 1H), 0.78-0.75 (m, 2H).

### Synthesis of 13.4 & 13

To a solution of 13.3 (170 mg, 468 μmol) in DCM (6 mL) at 0° C. was added silica gel (201 mg) and PCC (201 mg, 936 μmol). After stirring at 10° C. for 0.5 h, the reaction mixture was diluted with PE (3 mL) and filtered through a pad of silica gel. The filter cake was washed with DCM (3×6 mL) and the combined organic solution was filtered and concentrated in vacuum. The residue was purified by silica gel chromatography (0-20% of EtOAc in PE) to give the 13.4 (65 mg, 38.6%) and 13 (65 mg, 38.6%) both as solids.

13.4: ¹H NMR (400 MHz, CDCl₃) δ_{H} 2.49-2.45 (m, 1H), 2.13 (s, 3H), 1.85-1.76 (m, 4H), 1.75-1.69 (m, 3H), 1.69-1.56 (m, 5H), 1.51-1.39 (m, 6H), 1.34-1.18 (m, 6H), 1.15-1.02 (m, 3H), 0.93 (s, 3H), 0.92-0.83 (m, 6H); LC-ELSD/MS purity 99%, MS ESI calcd. for C₂₂H₃₉O [M+H-H₂O]+ 343.3, found 343.3.

13: ¹H NMR (400 MHz, CDCl3) δ_{H} 2.34-2.26 (m, 1H), 2.14 (s, 3H), 1.86-1.78 (m, 3H), 1.76-1.65 (m, 5H), 1.62-1.56 (m, 3H), 1.51-1.38 (m, 5H), 1.35-1.20 (m, 6H), 1.15-1.11 (m, 1H), 1.06-0.94 (m, 4H), 0.94-0.90 (m, 6H), 0.89-0.80 (m, 3H); LC-ELSD/MS purity 99%, MS ESI calcd. for C₂₂H₃₉O [M+H-H₂O]⁺343.3, found 343.3.

### Example 14: Synthesis of 1-(2-((1S,4aS,4bR,6aR,8R,11aS,11bR,13aS)-8-ethyl-8-hydroxy-13a-methyloctadecahydro-1H-cyclohepta[a]phenanthren-1-yl)-2-oxoethyl)-1H-pyrazole-4-carbonitrile

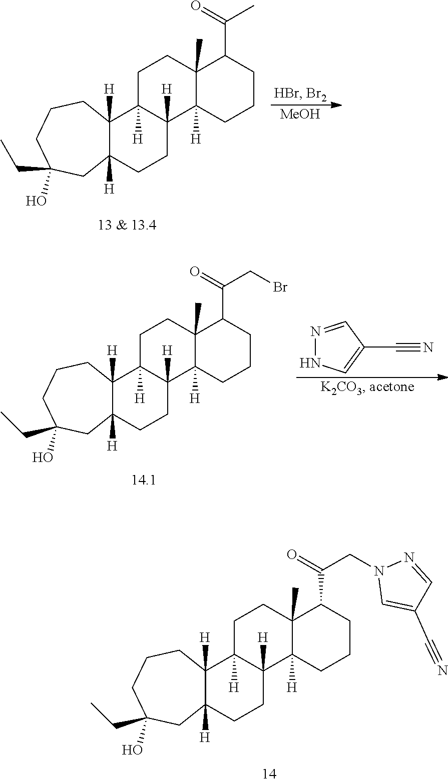

### Synthesis of 14.1

To a solution of a mixture of 13.4 & 13 (100 mg, 277 μmol) in methanol (2 mL) was added HBr (40%, 11 mg, 55.4 μmol) and Br₂ (48.5 mg, 304 gmol) dropwise at 25° C. After stirring for 2 h, the reaction mixture was diluted with NaHCO₃ (10 mL) and extracted with EtOAc (2×20 mL). The combined organic phase was washed with saturated brine (2×50 mL), dried over anhydrous Na₂SO₄, filtered and concentrated in vacuum to give 14.1 (121 mg) as a solid.

### Synthesis of 14

To a solution of 14.1 (121 mg, 0.275 mmol) in acetone (2 mL) was added 4-cyanopyrazole (30.6 mg, 0.329 mmol) and K₂CO₃ (75.8 mg, 0.55 mmol). After stirring at 25° C. for 4 h, the mixture was diluted with water (20 mL) and extracted with EtOAc (2×20 mL). The combined organic layer was concentrated, and the residue purified by flash column (0˜35% EtOAc in PE) to give only 14 (5 mg, 4.03%) as a solid. ¹H NMR (400 MHz, CDCl₃)^{6H} 7.83 (s, 1H), 7.81 (s, 1H), 5.07-4.93 (m, 2H), 2.33 (dd, J=3.2, 12.4 Hz, 1H), 1.85-1.70 (m, 8H), 1.50-1.42 (m, 5H), 1.37-1.29 (m, 5H), 1.29-1.23 (m, 7H), 1.20-1.14 (m, 2H), 1.07-1.01 (m, 2H), 0.96 (s, 3H), 0.93-0.88 (m, 4H); LC-ELSD/MS purity 99%, MS ESI calcd. for C₂₈H₄₀N₃O [M+H-H₂O]⁺434.3, found 434.3.

### Example 15: Synthesis of 1-((1S,4aS,4bR,6aR,8R,11aS,11bS,13aS)-8-hydroxy-8,11a,13a-trimethyloctadecahydro-1H-cyclohepta[a]phenanthren-1-yl)ethan-1-one

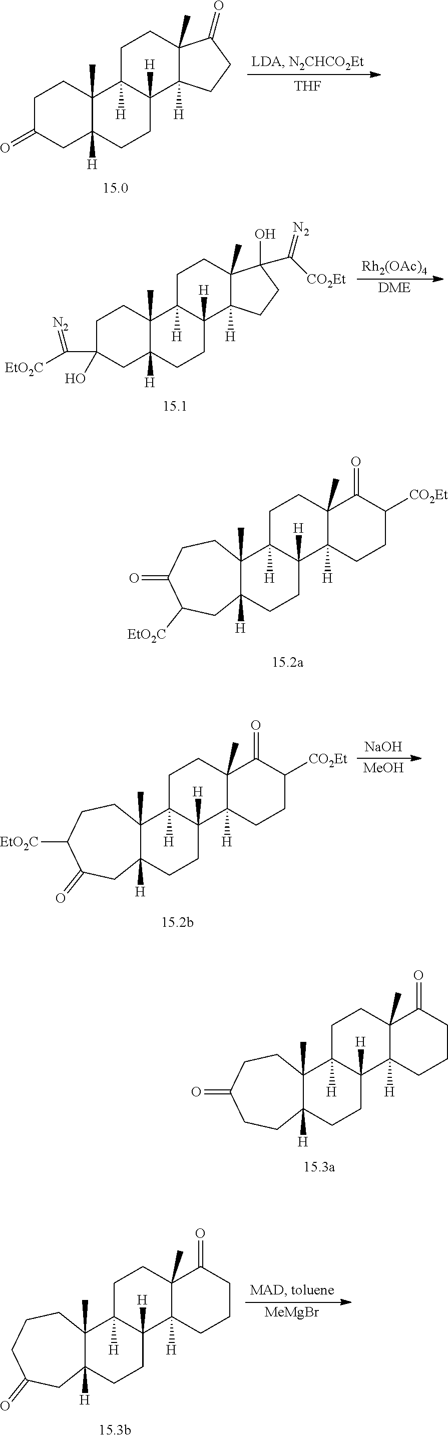

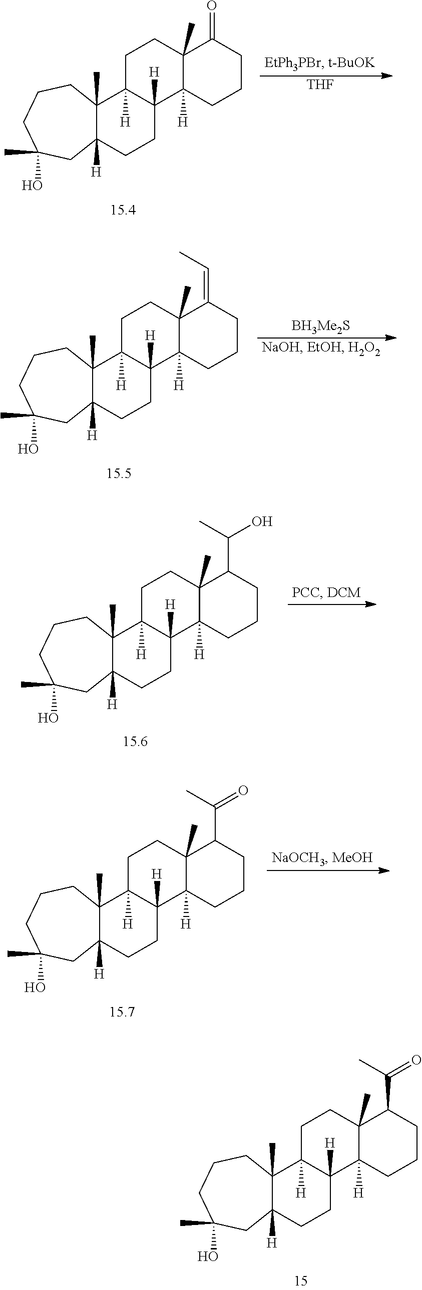

### Synthesis of 15.1

To a cold (−70° C.) LDA solution (138 mmol) was added to a solution of (5p)-androstane-3,17-dione, 15.0 (5 g, 17.3 mmol) and ethyl diazoacetate (15.7 g, 138 mmol) in THF (300 mL). After stirring at −70° C. for 2 h, the reaction was quenched with acetic acid (8.28 g, 138 mmol) in THF (50 mL). After warming to 25° C. for 16 h, the reaction mixture was diluted with water (800 mL) and PE (200 mL). The organic phase was separated, and the aqueous phase was extracted with EtOAc (300 mL). The combined organic layers were washed with saturated brine (1000 mL), dried over anhydrous Na₂SO₄, filtered and concentrated to give 15.1 (9.5 g) as an oil, which was used directly in next step.

### Synthesis of 15.2a & 15.2b

To a solution of 15.1 (9.5 g, 18.3 mmol) in DME (150 mL) was added Rh₂(OAc)₄ (161 mg, 0.37 mmol). After stirring at 25° C. for 16 h, the reaction mixture was concentrated. The residue was purified by silica gel chromatography (0-20% of EtOAc in PE) to give a mixture of diastereomers 15.2a & 15.2b (8.5 g) as an oil.

### Synthesis of 15.3a & 15.3b

To a mixture of 15.2a & 15.2b (8 g, 17.3 mmol) in MeOH (160 mL) was added H₂O (60 mL) and NaOH (5.52 g, 138 mmol). After stirring at 60° C. for 16 h, the reaction mixture was concentrated. The residue was diluted with H₂O (200 mL) and extracted with EtOAc (2×200 mL). The combined organic phase was washed with saturated brine (200 mL), dried over anhydrous Na₂SO₄, filtered and concentrated. The residue was purified by silica gel chromatography (0-15% of EtOAc in PE) to give a mixture of diastereomers 15.3a & 15.3b (3.9 g) as a solid.

The diastereomers were separated by SFC (Column: DAICEL CHIRALPAK AD (250 mm*50 mm, 10 um); Condition: 0.1% NH₃H₂O EtOH)-ACN; Begin B: 25; End B: 25) to give the product 15.3a (1 g) and the product 15.3b (2.2 g) as solids.

15.3a: ¹H NMR (400 MHz, CDCl₃) δ 2.67-2.56 (m, 1H), 2.53-2.38 (m, 3H), 2.34-2.15 (m, 2H), 2.11-2.04 (m, 2H), 1.95-1.64 (m, 6H), 1.63-1.29 (m, 10H), 1.23-1.17 (m, 1H), 1.09 (s, 3H), 1.01 (s, 3H), 0.99-0.87 (m, 1H).

15.3b: ¹H NMR (400 MHz, CDCl₃) δ 3.03 (t, J=12.8 Hz, 1H), 2.69-2.56 (m, 1H), 2.49-2.32 (m, 2H), 2.26-2.16 (m, 1H), 2.09-2.03 (m, 1H), 1.95-1.65 (m, 8H), 1.60-1.43 (m, 6H), 1.39-1.21 (m, 5H), 1.09 (s, 3H), 0.97 (s, 3H), 0.91-0.77 (m, 1H).

### Synthesis of 15.4

To a solution of 2,6-di-tert-butyl-4-methylphenol (8.32 mg, 37.8 mmol) in toluene (20 mL) was added dropwise AlMe₃ (9.45 mL, 18.9 mmol, 2 M in toluene) at 0° C. and stirred at 30° C. for 30 min “MAD solution”. To the fresh prepared MAD (18.9 mmol) solution in toluene (20 mL) was added 15.3b (2 g, 6.31 mmol) in DCM (20 mL) dropwise at −70° C. After stirring at −70° C. for 1 h under N₂, MeMgBr (6.30 mL, 18.9 mmol) was added dropwise at −70° C. After stirring at −70° C. for 4 h, the reaction mixture was poured into saturated aqueous citric acid (200 mL) at 10° C. and extracted with EtOAc (2×100 mL). The combined organic layer was dried over Na₂SO₄, filtered and concentrated in vacuum. The residue was purified by flash column (0-30% of EtOAc in PE) to give product 15.4 (870 mg, 41.7%) as a solid. ¹H NMR (400 MHz, CDCl₃)⁶H 2.67-2.56 (m, 1H), 2.23-2.15 (m, 1H), 2.09-1.95 (m, 2H), 1.87-1.69 (m, 5H), 1.68-1.58 (m, 2H), 1.53-1.29 (m, 8H), 1.28-1.16 (m, 7H), 1.14-0.93 (m, 6H), 0.89 (s, 3H), 0.87-0.76 (m, 1H). LC-ELSD/MS: purity>99%, MS ESI calcd. for C₂₂H₃₂ [M-2H₂O]+ 297.2, found 297.2.

### Synthesis of 15.5

To a suspension of Ph₃PEtBr (1.33 g, 3.6 mmol) in anhydrous THF (30 mL) was added t-BuOK (403 mg, 3.6 mmol) at 25° C. under N₂. After stirring at 60° C. for 30 min, a solution of 15.4 (200 mg, 0.601 μmol) in anhydrous THE (10 mL) was added dropwise. After stirring at 60° C. for 16 h, the reaction mixture was poured into saturated NH₄Cl (100 mL), stirred for 10 min, and the aqueous phase was extracted with EtOAc (2×50 mL). The combine organic phase was washed with saturated brine (2×100 mL), filtered and concentrated. The residue was purified by flash column to give 15.5 (100 mg) as a solid. 1H NMR (400 MHz, CDCl₃)^{6H} 5.21-5.11 (m, 1H), 2.54-2.44 (m, 1H), 2.20-1.96 (m, 2H), 1.88-1.66 (m, 7H), 1.48-1.31 (m, 8H), 1.30-1.11 (m, 14H), 0.92 (s, 3H), 0.89-0.78 (m, 4H).

### Synthesis of 15.6

To a solution of 15.5 (350 mg, 1.01 mmol) in THF (10 mL) was added BH₃Me₂S (0.303 mL, 10 M 3.03 mmol). After stirring at 45° C. for 1 h, the reaction mixture was diluted with ethanol (694 mg, 15.1 mmol) at 15° C., followed by NaOH aqueous (3.02 mL, 5.0 M, 15.1 mmol) at 15° C. and then H₂O₂ (1.51 mL, 10 M, 15.1 mmol) dropwise at 15° C. After stirring at 78° C. for 1 h, the mixture was cooled to 15° C., poured into water (100 mL) and extracted with EtOAc (2×50 mL), The combined organic layer was washed with saturated brine (2×200 mL), dried over anhydrous Na₂SO₄, filtered and concentrated in vacuum to give 15.6 (350 mg) as a solid.

### Synthesis of 15.7

To a solution of 15.6 (350 mg, 0.965 mmol) in DCM (10 ml) were added PCC (412 mg, 1.92 mmol) and silica gel (450 mg). After stirring at 25° C. for 1 h, the mixture was filtered and concentrated. The residue was purified by flash column (0-30% of EtOAc in PE) to give product 15.7 (200 mg, 57.6%) as a solid. ¹H NMR (400 MHz, CDCl3) S_{H} 3.78-3.70 (m, 2H), 2.48-2.25 (m, 1H), 2.17-2.11 (m, 3H), 2.07-1.97 (m, 2H), 1.89-1.76 (m, 4H), 1.75-1.58 (m, 5H), 1.53-1.32 (m, 8H), 1.28-1.15 (m, 9H), 0.92 (s, 3H), 0.87 (s, 3H).

### Synthesis of 15

A solution of 15.7 (50 mg, 0.138 mmol) and MeONa (74.5 mg, 1.38 mmol) in MeOH (10 mL) was stirred at 70° C. for 2 days. The reaction mixture was pure into water and extracted with EtOAc (2×10 ml). The combined organic solution was dried over with anhydrous Na₂SO₄, filtered and concentrated in vacuum. The residue was purified by flash column (0-30% of EtOAc in PE) to give product 15 (10.1 mg, 20.3%) as a solid. ¹H NMR (400 MHz, CDCl₃) δ S_{H}2.32-2.25 (m, 1H), 2.14 (s, 3H), 2.07-1.99 (m, 1H), 1.87-1.59 (m, 8H), 1.52-1.34 (m, 6H), 1.33-1.18 (m, 9H), 1.17-1.01 (m, 4H), 0.91 (s, 3H), 0.87 (s, 3H), 0.83-0.70 (m, 2H). LC-ELSD/MS: purity>99%, MS ESI calcd. for C₂₄H₃₈O [M−H₂O]⁺ 343.3, found 343.3.

### Example 16 & 17. Synthesis of 1-(2-((1S,4aS,4bR,6aR,8R,11aS,11bS,13aS)-8-hydroxy-8,11a,13a-trimethyloctadecahydro-1H-cyclohepta[a]phenanthren-1-yl)-2-oxoethyl)-1H-pyrazole-4-carbonitrile & 1-(2-((1R,4aS,4bR,6aR,8R,11aS,11bS,13aS)-8-hydroxy-8,11a,13a-trimethyloctadecahydro-1H-cyclohepta[a]phenanthren-1-yl)-2-oxoethyl)-1H-pyrazole-4-carbonitrile

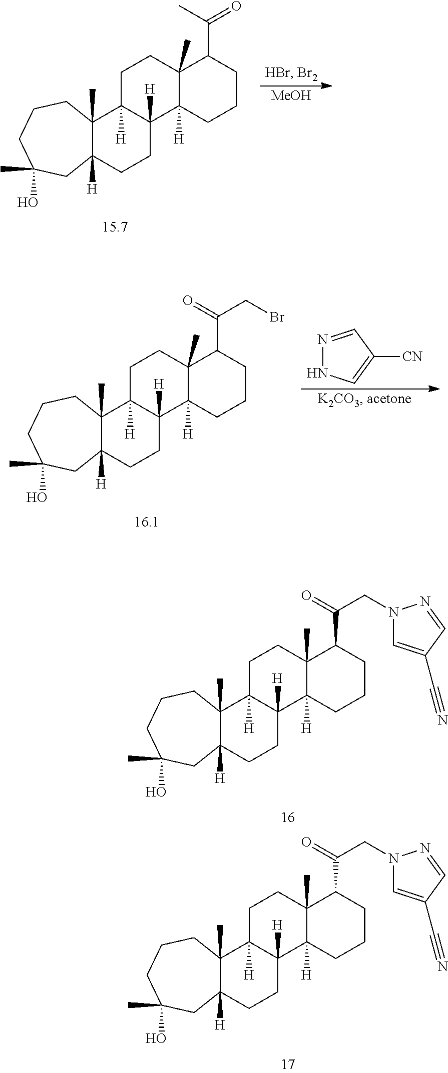

### Synthesis of 16.1

To a solution of 15.7 (150 mg, 0.415 mmol) and HBr (16.6 mg, 0.083 mmol, 40%) in MeOH (10 mL) was added Br₂ (72.9 mg, 0.456 mmol) at 0° C. After stirring at 20° C. for 2 h, the mixture was added into saturated NaHCO₃ (50 mL) and extracted with EtOAc (3×20 mL). The combined organic layer was washed with saturated brine (50 mL), dried over anhydrous Na₂SO₄, filtered and concentrated to give 16.1 (150 mg) as an oil. ¹H NMR (400 MHz, CDCl₃) δ_{H} 4.01-3.85 (m, 2H), 2.75-2.51 (m, 1H), 2.03-1.60 (m, 11H), 1.49-1.28 (m, 11H), 1.22-1.01 (m, 8H), 0.96-0.93 (m, 3H), 0.87 (s, 3H).

### Synthesis of 16 & 17

To a solution of 16.1 (100 mg, 0.227 mmol) in acetone (5 ml) were added K₂CO₃ (62.6 mg, 0.454 mmol) and 1H-pyrazole-4-carbon (42.2 mg, 0.454 mmol). After stirring at 25° C. for 2 h, the reaction mixture was poured into water and extracted with EtOAc (2×20 ml). The combined organic solution was dried over anhydrous Na₂SO₄, filtered and concentrated. The residue was purified by flash column (0-30% of EtOAc in PE) to give product 17 (50 mg) and 16 (23.9 mg, 23.4%) as solids. Compound 17(50 mg) was further purified by HPLC (Column: Welch XtimateC18 150*25 mm*5 um); condition: water (0.04% NH₃H₂O)-ACN; Begin B: 70%; End B: 100%) to afford 17 (5.4 mg, 10.8%) as a solid.

16: ¹H NMR (400 MHz, CDCl₃) δ_{H} 7.82 (s, 1H), 7.81 (s, 1H), 5.08-4.90 (m, 2H), 2.36-2.28 (m, 1H), 2.07-1.98 (m, 1H), 1.91-1.67 (m, 7H), 1.54-1.36 (m, 7H), 1.34-1.23 (m, 9H), 1.20-0.97 (m, 6H), 0.95 (s, 3H), 0.87 (s, 3H); LC-ELSD/MS: purity>99%, MS ESI calcd. for C₂₈H₃₉N₃O [M−H₂O]⁺ 434.3 found 434.3.

17: ¹H NMR (400 MHz, CDCl₃) δ_{H}7.86 (s, 1H), 7.83 (s, 1H), 5.08-4.83 (m, 2H), 2.45 (d, J=6.2 Hz, 1H), 2.04-1.94 (m, 1H), 1.92-1.58 (m, 10H), 1.51-1.26 (m, 10H), 1.25-1.06 (m, 9H), 0.98 (s, 3H), 0.86 (s, 3H); LC-ELSD/1S: purity>99%, MS ESI calcd. for C₂₈H₃₉N₃O [M−H₂O]⁺ 434.3 found 434.3.

### Example 18: Synthesis of 1-(3-((1S,4aS,4bR,6aR,8R,11aS,11bS,13aS)-8-hydroxy-8,11a,13a-trimethyloctadecahydro-1H-cyclohepta[a]phenanthren-1-yl)-3-oxopropyl)-1H-pyrazole-4-carbonitrile

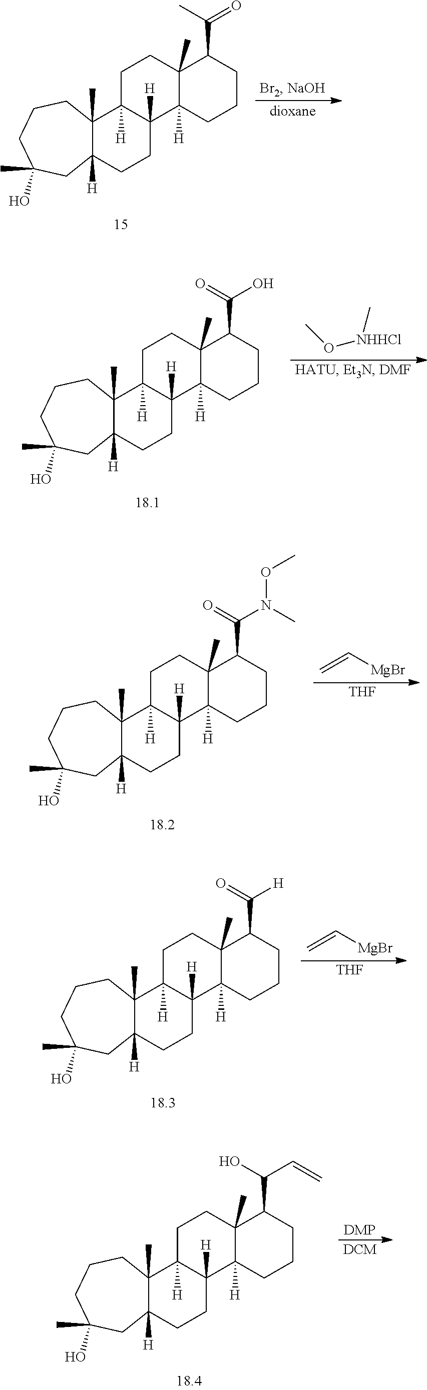

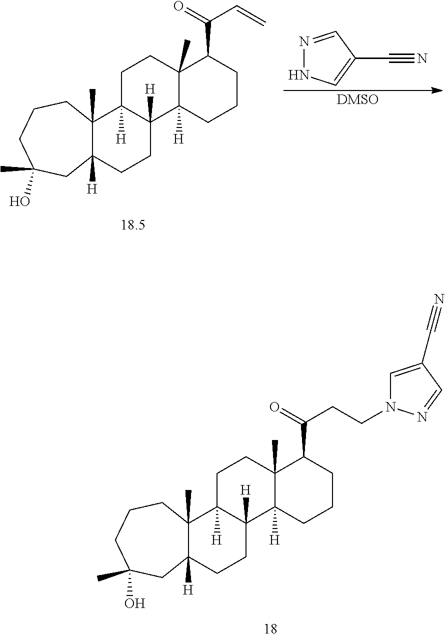

### Synthesis of 18.1

Liquid bromine (1.05 g, 6.60 mmol) was added slowly to a vigorously stirred sodium hydroxide aqueous (7.33 mL, 3 M, 22 mmol) at 0° C. When all the bromine was dissolved, the mixture was added slowly to a stirred solution of 15 (400 mg, 1.10 mmol) in dioxane (10 mL) and water (2 mL). After stirring at 15° C. for 16 h, the reaction mixture was by Na₂SO₃ aqueous (3 mL), and pH adjusted with hydrochloride acid (3 N) whereby a solid precipitated from solution. The solid was dissolved in EtOAc (10 mL). The organic phase was separated, and the aqueous phase was extracted EtOAc (2×10 mL). The combined organic was washed with saturated brine (30 mL), dried over anhydrous Na₂SO₄, filtered and concentrated in vacuum to give 18.1 (400 mg) as a solid. ¹H NMR (400 MHz, CDCl₃) δ_{H} 2.16-1.98 (m, 2H), 1.88-1.76 (m, 3H), 1.73-1.46 (m, 8H), 1.45-1.27 (m, 6H), 1.26-1.01 (m, 11H), 1.01-0.91 (m, 4H), 0.89-0.84 (m, 3H)

### Synthesis of 18.2

To a solution of 18.1 (400 mg, 1.14 mmol) in DMF (5 mL) was added N,O-dimethylhydroxylamine hydrochloride (444 mg, 4.56 mmol), HATU (866 mg, 2.80 mmol) and TEA (1.15 g, 11.4 mmol) at 15° C. After stirring at 15° C. for 2 h, the reaction mixture was diluted with water (20 mL) and extracted with ethyl acetate (30 mL×3). The combined organic phase was washed with water (50 mL×2), dried over Na₂SO₄, filtered, concentrated under vacuum. The residue was purified by flash column (0-30% of EtOAc in PE) to give 18.2 (250 mg, 56%) as a solid. ¹H NMR (400 MHz, CDCl₃) δ_{H} 3.67 (s, 3H), 3.17 (s, 3H), 2.08-1.99 (m, 2H), 1.93-1.59 (m, 8H), 1.55-1.40 (m, 6H), 1.38-1.23 (m, 12H), 1.16-1.10 (m, 2H), 1.01 (s, 3H), 0.87 (s, 4H).

### Synthesis of 18.3

To a solution of 18.2 (250 mg, 0.62 mmol) in THE (10 mL) was added CH₂CHMgBr (2 mL, 1.6 M, 3.20 mmol) at 20° C. The mixture was stirred at 20° C. for 2 hours. The mixture was added into saturated NH₄Cl (100 mL). The aqueous layer was extracted with EtOAc (3×50 mL). The combined organic layer was washed with saturated brine (100 mL), dried over anhydrous Na₂SO₄, filtered and concentrated. The residue was purified by flash column (0-10% of EtOAc in PE) to give 18.3 (100 mg, 47%) as an oil. 1H NMR (400 MHz, CDCl₃) S_{H} 9.81 (d, J=2.4 Hz, 1H), 2.10-1.94 (m, 2H), 1.90-1.59 (m, 7H), 1.52-1.28 (m, 8H), 1.26 (s, 3H), 1.25-0.95 (m, 8H), 0.94 (s, 3H), 0.88 (s, 3H), 0.86-0.75 (m, 3H).

### Synthesis of 18.4

To a solution of 18.3 (100 mg, 0.29 mmol) in THF (2 mL) was added CH₂CHMgBr (1.4 mL, 1.40 mmol, 1.0 M in THF) at 20° C. After stirring at 20° C. for 1 h, the mixture was added into saturated NH₄Cl (50 mL) and extracted with EtOAc (3×20 mL). The combined organic layer was washed with saturated brine (100 mL), dried over anhydrous Na₂SO₄, filtered and concentrated to give 18.4 (110 mg) as an oil. ¹H NMR (400 MHz, CDCl₃) δ_{H} 5.95-5.80 (m, 1H), 5.18 (d, J=17.2 Hz, 1H), 5.09 (d, J=10.4 Hz, 1H), 4.54 (s, 1H), 3.75-3.65 (m, 1H), 2.03-1.93 (m, 2H), 1.90-1.59 (m, 8H), 1.52-1.27 (m, 8H), 1.25 (s, 3H), 1.24-0.95 (m, 8H), 0.94 (s, 3H), 0.88 (s, 3H), 0.80-0.70 (m, 2H).

### Synthesis of 18.5

A solution of 18.4 (90 mg, 0.24 mmol) and DMP (203 mg, 0.48 mmol) in DCM (10 mL) was stirred at 20° C. for 5 minutes. The mixture was added into saturated NaHCO₃/Na₂S₂O₃ (50 mL/50 mL). The organic layer was separated and washed with saturated brine (50 mL), dried over anhydrous Na₂SO₄, filtered and concentrated. The residue was purified by flash column (0-15% of EtOAc in PE) to give 18.5 (40 mg, 45%) as an oil.

¹H NMR (400 MHz, CDCl₃) δ_{H}6.40 (dd, J=10.0 Hz, 17.2 Hz, 1H), 6.18 (d, J=17.2 Hz, 1H), 5.66 (d, J=10.4 Hz, 1H), 2.54 (dd, J=2.8 Hz, 12.0 Hz, 1H), 2.03 (dd, J=9.6 Hz, 14.0 Hz, 1H), 1.90-1.59 (m, 10H), 1.52-1.28 (m, 9H), 1.26 (s, 3H), 1.25-0.94 (m, 5H), 0.91 (s, 3H), 0.86 (s, 3H), 0.84-0.70 (m, 2H).

### Synthesis of 18

To a solution of 18.5 (40 mg, 0.11 mmol) in DMSO (5 mL) were added 1-methyl-1H-imidazole (26.0 mg, 0.32 mmol) and 1H-pyrazole-4-carbonitrile (19.9 mg, 0.21 mmol). After stirring at 70° C. for 16 h, the mixture was added into saturated brine (50 mL). and extracted with EtOAc (3×20 mL). The combined organic layer was washed with saturated brine (2×50 mL), dried over anhydrous Na₂SO₄, filtered and concentrated. The residue was purified by flash column (0-50% of EtOAc in PE) to give 18 (13 mg, 26%) as a solid. 1H NMR (400 MHz, CDCl₃) S_{H} 7.89 (s, 1H), 7.75 (s, 1H), 4.45-4.35 (m, 2H), 3.10-3.00 (m, 2H), 2.22 (dd, J=2.8 Hz, 12.8 Hz, 1H), 2.01 (dd, J=9.6 Hz, 13.6 Hz, 1H), 1.90-1.59 (m, 7H), 1.52-1.26 (m, 7H), 1.25 (s, 3H), 1.24-0.86 (m, 10H), 0.85-0.84 (m, 6H), 0.83-0.65 (m, 2H). LC-ELSD/MS: purity>99%, MS ESI calcd. for C₂₉H₄₂N₃O [M−H₂O+H]⁺ 448.3, found 448.3.

### Example 19: Synthesis of 1-(2-((1S,4aS,4bS,6aR,8R,10aS,10bS,12aS)-10a-ethyl-8-hydroxy-8,12a-dimethyloctadecahydrochrysen-1-yl)-2-oxoethyl)-1H-pyrazole-4-carbonitrile (19)

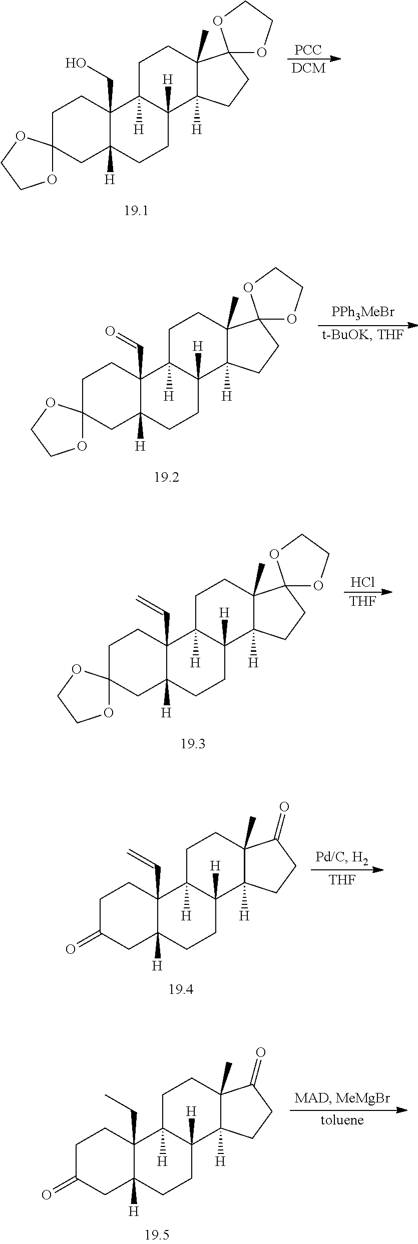

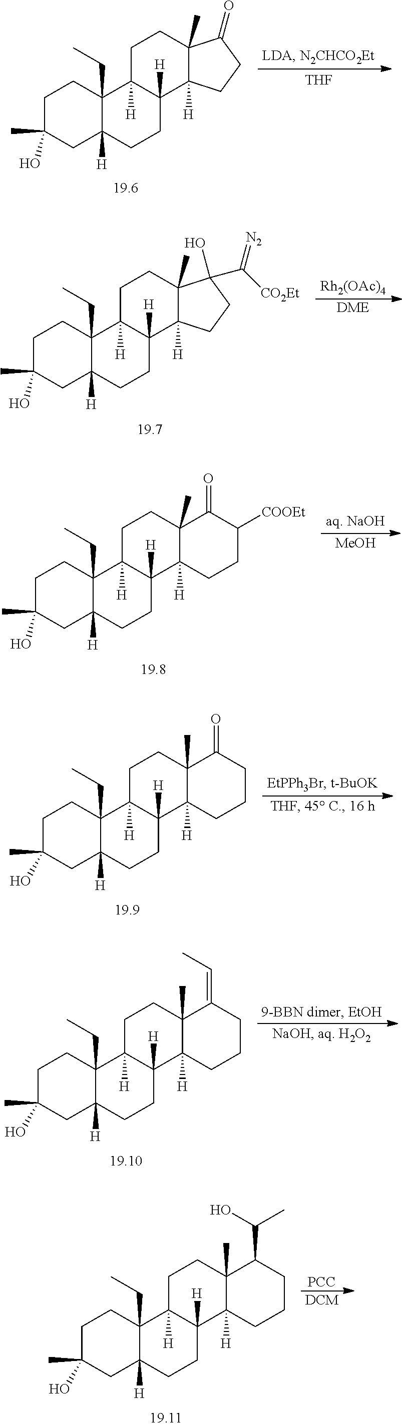

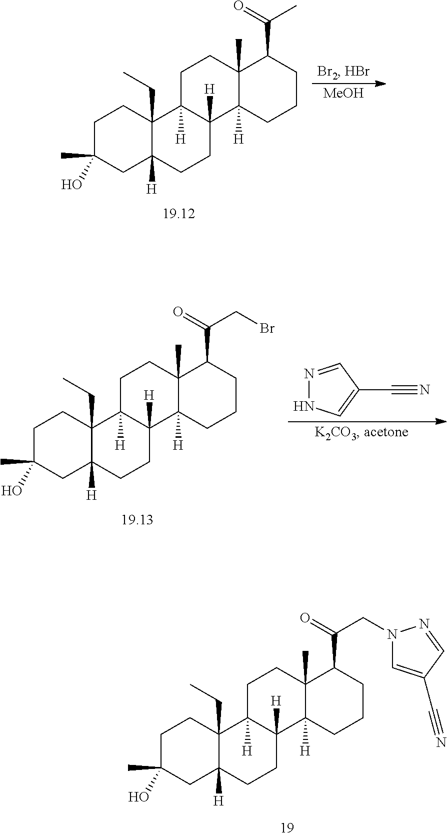

### Synthesis of 19.2

To a solution of 19.1 (10.0 g, 25.4 mmol, reported in patent ‘WO2016/134301, 2016, A2’) in DCM (100 mL) was added silica gel (10.0 g) and PCC (8.17 g, 38 mmol), and the mixture was stirred at RT for 1 h. The suspension was filtered, and the filter cake was washed with DCM (2×100 mL). The combined filtrate was concentrated to give 19.2 (10.0 g crude). ¹H NMR (400 MHz, CDCl₃) S_{H} 9.56 (s, 1H), 4.00-3.80 (m, 8H), 2.24-1.88 (m, 5H), 1.87-1.70 (m, 5H), 1.46-1.35 (m, 5H), 1.33-0.99 (m, 5H), 0.92 (s, 3H), 0.89-0.69 (m, 2H).

### Synthesis of 19.3

At 15° C., to a mixture of MePPh₃Br (27.1 g, 76.1 mmol) in THF (100 mL) was added t-BuOK (8.53 g, 76.1 mmol). The resulting mixture was warmed to 50° C. and stirred for 30 min. 19.2 (9.91 g, 25.4 mmol) was added in small portions and the reaction mixture was stirred at 50° C. for another 1 h. The reaction was cooled to 15° C. and quenched with 10% NH₄Cl aqueous solution (200 mL). The layers were separated, and the aqueous layer was extracted with EtOAc (300 mL). The combined organic phase was concentrated, and the residue was purified by silica gel chromatography (PE/EtOAc=20/1 to 5/1) to afford 19.3 (5.0 g, 50.7%). ¹H NMR (400 MHz, CDCl₃) δ_{H} 6.32-6.25 (m, 1H), 5.15-4.94 (m, 2H), 3.95-3.80 (m, 8H) 2.02-1.72 (m, 6H), 1.69-1.61 (m, 1H), 1.59-1.31 (m, 12H), 1.21-1.04 (m, 3H), 0.80 (s, 3H).

### Synthesis of 19.4

To a solution of 19.3 (15.0 g, 12.8 mmol) in THF (30 mL) was added aqueous HCl (38.6 mL, 2M, 77.2 mmol). The mixture was stirred at RT for 5 h then quenched with saturated NaHCO₃ (100 mL). The layers were separated, and the aqueous layer was extracted with EtOAc (2×100 mL). The combined organic layer was washed with brine (30 mL), dried over anhydrous Na₂SO₄, filtered and concentrated to give 19.4 (9.30 g, 80.8%). ¹H NMR (400 MHz, CDCl₃) S_{H} 6.35-6.25 (m, 1H), 5.19 (d, J=11.2 Hz, 1H), 5.09 (d, J=18.0 Hz, 1H), 2.79-2.64 (m, 1H), 2.54-2.13 (m, 5H), 2.13-2.05 (m, 3H), 2.02-1.79 (m, 3H), 1.69-1.50 (m, 6H), 1.37-1.23 (m, 4H), 0.87 (s, 3H).

### Synthesis of 19.5

To a solution of 19.4 (11.0 g, 36.6 mmol) in THF (200 mL) was added Pd/C (wet, 50%, 2.0 g). The suspension was degassed under vacuum and purged with H₂ for three times. The mixture was stirred under H₂ (30 psi) at RT for 16 h to give a black suspension. The reaction mixture was filtered through a pad of Celite and washed with THF (2×100 mL). The filtrate was concentrated, and the residue was triturated from PE (300 mL) to give 19.5 (12.0 g, crude). ¹H NMR (400 MHz, CDCl₃)^{6H} 2.67 (t, J=13.60 Hz, 1H), 2.52-2.06 (m, 5H), 2.00-1.91 (m, 1H), 1.89-1.48 (m, 12H), 1.39-1.19 (m, 5H), 0.87 (s, 3H), 0.80 (t, J=7.53 Hz, 3H).

### Synthesis of 19.6

At 0° C., to a solution of 2,6-di-tert-butyl-4-methylphenol (43.6 g, 198 mmol) in toluene (40 mL) was added AlMe₃ (49.5 mL, 99.0 mmol, 2 M in toluene) dropwise, and the mixture was stirred at RT for 30 min. The freshly made MAD (47.5 g, 99.0 mmol) solution was cooled to −70° C. and a solution of 19.5 (10.0 g, 33.0 mmol) in anhydrous DCM (30 mL) was added dropwise. After stirring at −70° C. for 1 h, MeMgBr (33.0 mL, 99.0 mmol, 3M in ethyl ether) was added dropwise at −70° C., then stirred for another 1 h. The reaction mixture was poured into saturated aqueous citric acid solution (50 mL) and the suspension was extracted with EtOAc (3×50 mL). The combined organic layers were washed with brine (50 mL), dried over anhydrous Na₂SO₄, and concentrated. The residue was purified by flash column (10˜30% of EtOAc in PE) to give 19.6 (9.10 g, 86.5%). ¹H NMR (400 MHz, CDCl₃) δ_{H} 2.43 (dd, J=8.0, 19.6 Hz, 1H), 2.13-2.00 (m, 1H), 1.98-1.87 (m, 2H), 1.83-1.32 (m, 15H), 1.27-1.12 (m, 9H), 0.88-0.77 (m, 6H).

### Synthesis of 19.7

At −70° C., a cooled (−70° C.) LDA solution (157 mL, 2 M, 314 mmol) was added to a stirred solution of 19.6 (20.0 g, 62.7 mmol) and ethyl diazoacetate (35.7 g, 313 mmol) in THF (500 mL). The mixture was stirred at −70° C. for 2 h, then HOAc (18.7 g, 313 mmol) in THF (50 mL) was added. The mixture was allowed to warm to RT and stirred for 16 h. Water (1000 mL) and PE (700 mL) was added, and the layers were separated. The aqueous layer was extracted with EtOAc (500 mL) and the combined organic layers were washed with saturated brine (1000 mL), dried over anhydrous Na₂SO₄, filtered and concentrated. The residue was purified by flash column (0-25% of EtOAc in PE) to give product 19.7 (28.8 g, crude).

### Synthesis of 19.8

To a solution of 19.7 (28.0 g, 64.7 mmol) in DME (300 mL) was added Rh₂(OAc)₄ (570 mg, 1.29 mmol). The reaction mixture was stirred at RT for 16 h to give a brown solution. The reaction mixture was concentrated, and the residue was purified by silica gel chromatography (0-20% of EtOAc in PE) to give the product 19.8 (27.0 g, crude).

### Synthesis of 19.9

To a mixture of 19.8 (27.0 g, 66.7 mmol) in MeOH (400 mL) was added H₂O (100 mL) and NaOH (13.3 g, 333 mmol). The reaction mixture was stirred at 60° C. for 16 h, then cooled and concentrated. H₂O (500 mL) and EtOAc (500 mL) were added, and the layers were separated. The aqueous layer was extracted with EtOAc (500 mL) and the combined organic phase was washed with saturated brine (500 mL), dried over anhydrous Na₂SO₄, filtered and concentrated. The residue was purified by silica gel chromatography (0-30% of EtOAc in PE) to give the product 19.9 (15.5 g, crude). ¹H NMR (400 MHz, CDCl₃) S_{H} 2.67-2.54 (m, 1H), 2.25-2.14 (m, 1H), 2.08-2.04 (m, 1H), 1.93-1.68 (m, 4H), 1.65-1.36 (m, 14H), 1.26-1.17 (m, 9H), 1.06 (s, 3H), 0.79 (t, J=7.6 Hz, 3H).

### Synthesis of 19.10

A solution of EtPPh₃Br (6.66 g, 18.0 mmol) and t-BuOK (2.01 g, 18.0 mmol) in THF (30 mL) was stirred at RT for 1 h. 19.9 (2.00 g, 6.01 mmol) in THF (10 mL) was added and the reaction mixture was stirred at 35° C. for 16 h. The mixture was poured into water (100 mL) and the layers were separated. The aqueous phase was extracted with EtOAc (3×100 mL) and the combined organic phase was washed with brine (2×100 mL), dried over anhydrous Na₂SO₄, filtered and concentrated. The residue was triturated with CH₃OH (100 mL) and water (100 mL) to give 19.10 (2.10 g, 81%). ¹HNMR (400 MHz, CDCl₃) δ 5.14 (q, J=7.6 Hz, 1H), 2.49 (br d, J=13.6 Hz, 1H), 2.31-2.08 (m, 1H), 2.02-1.80 (m, 3H), 1.78-1.69 (m, 5H), 1.65-1.60 (m, 2H), 1.48-1.37 (m, 9H), 1.22-1.12 (m, 10H), 1.06-1.00 (m, 3H), 0.93-0.87 (m, 2H), 0.77 (t, J=7.6 Hz, 3H).

### Synthesis of 19.11

A solution of 19.10 (2.10 g, 6.09 mmol and 9-BBN dimer (4.44 g, 18.2 mmol) in THF (30 mL) was stirred at 50° C. for 1 h, then cooled to 0° C. NaOH (9.74 mL, 5M in water, 48.7 mmol) was added, followed by hydrogen peroxide (4.87 mL, 48.7 mmol) and the reaction was warmed to 78° C. After stirring at 78° C. for 3 h, saturated aqueous Na₂S₂O₃ (50 mL) and ice-water (100 mL) were added. The suspension was extracted with EtOAc (3×100 mL) and the combined organic phase was washed with brine (2×100 mL), dried over anhydrous Na₂SO₄, filtered and concentrated. The residue was triturated with CH₃OH (100 mL) and water (500 mL) to give 19.11 (2.00 g, curde). ¹HNMR (400 MHz, CDCl₃) δ 3.83-3.72 (m, 1H), 1.91-1.76 (m, 7H), 1.66-1.55 (m, 12H), 1.44-1.33 (m, 10H), 1.18-1.05 (m, 5H), 0.97-0.87 (m, 3H), 0.76-0.71 (m, 3H).

### Synthesis of 19.12

To a solution of 19.11 (2.00 g, 5.51 mmol) and PCC (3.55 g, 16.5 mmol) in DCM (30 mL) was added silica gel (15.00 g). The reaction mixture was stirred at RT for 3 h, then concentrated. The residue was purified by silica gel chromatography (PE/EtOAc=3/1) to afford product (800 mg). The crude product was dissolved in EtOAc (30 mL), washed with saturated NH₄Cl (25 mL), NaOH aqueous solution (25 mL, 10%) and dried over anhydrous Na₂SO₄ and filtered. Evaporation of the solvent under reduced pressure to give 19.12 (350 mg, 44%). ¹H NMR (400 MHz, CDCl₃) δ 4.33 (t, J=6.0 Hz, 1H), 2.29 (dd, J=3.2, 12.8 Hz, 11H), 2.13 (s, 3H), 2.01-1.87 (m, 2H), 1.85-1.78 (m, 1H), 1.65-1.55 (m, 15H), 1.45-1.32 (m, 10H), 0.92-0.86 (m, 4H), 0.80-0.72 (m, 3H).

### Synthesis of 19.13

To a solution of 19.12 (330 mg, 0.915 mmol) in MeOH (20 ml) was added HBr (37.0 mg, 0.183 mmol, 40% in water) and Br₂ (149 mg, 0.933 mmol). After stirring at RT for 2 h, the mixture was quenched with saturated aqueous NaHCO₃ (50 mL), then extracted with EtOAc (2×50 mL). The combined organic phase was washed with brine (20 mL), dried over anhydrous Na₂SO₄, filtered and concentrated to afford 19.13 (260 mg, 65%), which was used directly to the next step. ¹H NMR (400 MHz, CDCl₃) δ 4.02-3.84 (m, 2H), 2.61-2.50 (m, 1H), 1.98-1.77 (m, 4H), 1.98-1.77 (m, 1H), 1.74-1.46 (m, 15H), 1.44-1.35 (m, 7H), 1.18-1.07 (m, 2H), 1.04-0.97 (m, 1H), 0.92 (s, 3H), 0.79-0.74 (m, 3H)

### Synthesis of 19

To a suspension of 19.13 (130 mg, 0.295 mmol) and K₂CO₃ (81.7 mg, 0.591 mmol) in acetone (5 mL) was added 1H-pyrazole-4-carbonitrile (55.0 mg, 0.591 mmol). The reaction was stirred at RT for 16 h then poured into water (50 mL). The suspension was extracted with EtOAc (3×50 mL) and the combined organic phase was washed with brine (2×50 mL), dried over anhydrous Na₂SO₄, filtered and concentrated. The residue was purified by silica gel chromatography (PE/EtOAc=30/1 to 1/2) to afford 19 (36.0 mg, 32%). ¹H NMR (400 MHz, CDCl₃) δ 7.82 (d, J=4 Hz, 2H), 5.11-4.89 (m, 2H), 2.32 (dd, J=3.2, 12.8 Hz, 1H), 1.99-1.81 (m, 2H), 1.81-1.68 (m, 3H), 1.67-1.57 (m, 7H), 1.47-1.32 (m, 7H), 1.31-1.19 (m, 811), 1.14 (dd, J=7.2, 14.0 Hz, 1H), 1.05-0.96 (m, 2H), 0.93 (s, 3H), 0.78 (t, J=7.2 Hz, 3H). LC-ELSD/MS purity 99%, MS ESI calcd. for C₂₅H₄₀N₃O [M+H-H₂O]⁺ 434.3, found 434.3.

### Example 20: Synthesis of 1-(2-((1S,3aS,3bS,5aR,7S,10aS,10bS,12aS)-10a-ethyl-7-hydroxy-7,12a-dimethyloctadecahydrocyclohepta[a]cyclopenta[f]naphthalen-1-yl)-2-oxoethyl)-1H-pyrazole-4-carbonitrile (20)

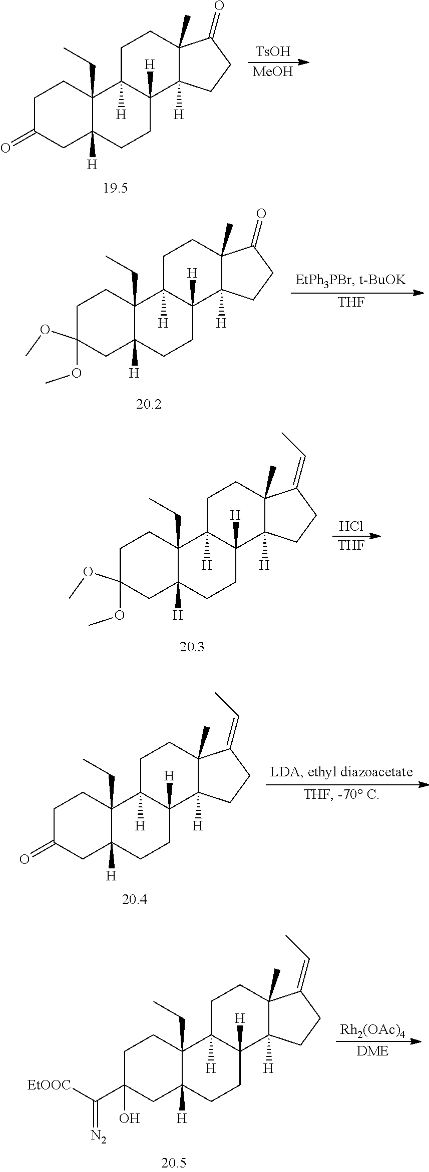

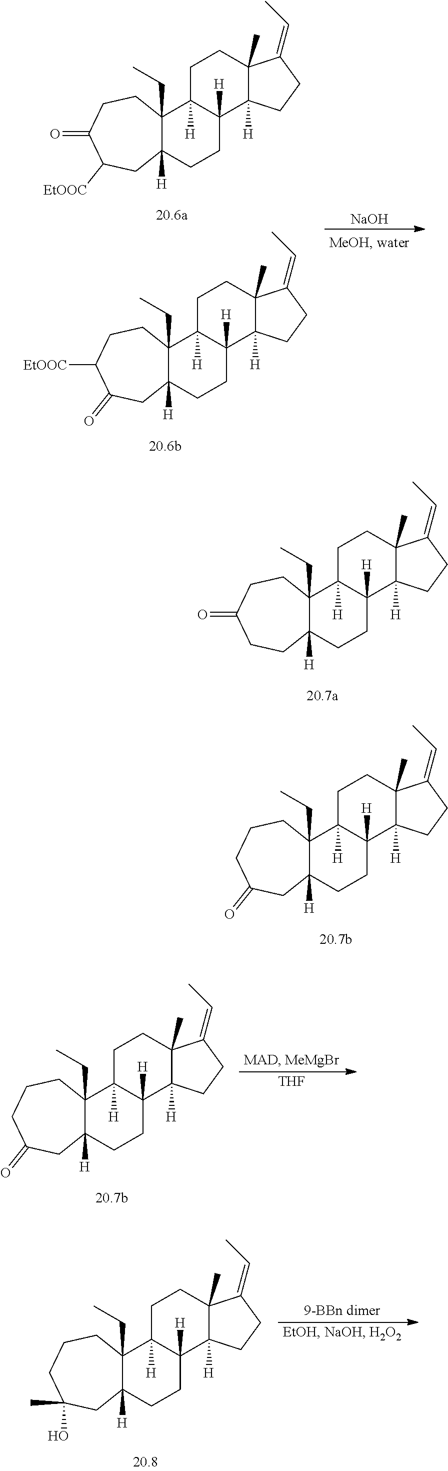

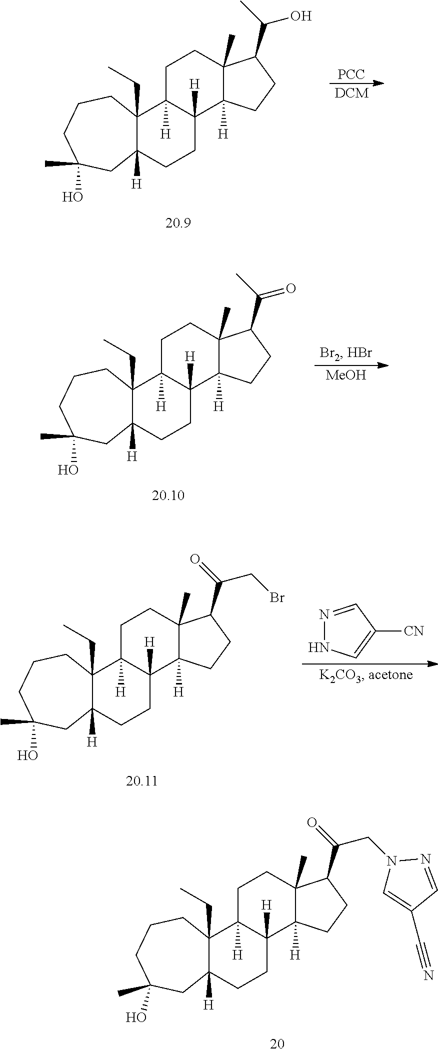

### Synthesis of 20.2

To a solution of 19.5 (15.0 g, 49.5 mmol) in MeOH (100 mL) was added TsOH (852 mg, 4.95 mmol) and the mixture was stirred at 60° C. for 18 h. The mixture was cooled to RT and Et₃N (14.9 g, 148 mmol) was added. The reaction mixture was concentrated to give 20.2 (17.2 g, crude). ¹H NMR (400 MHz, CDCl₃)⁶H 3.11-3.25 (m, 6H), 2.84-3.00 (m, 3H), 2.38-2.49 (m, 1H), 2.00-2.15 (m, 2H), 1.86-1.98 (m, 2H), 1.43-1.72 (m, 10H), 1.34-1.42 (m, 3H), 0.76-0.86 (m, 7H).

### Synthesis of 20.3

To a suspension of Ph₃PEtBr (36.6 g, 98.6 mmol) in anhydrous THF (400 mL) was added t-BuOK (11.0 g, 98.6 mmol) and the mixture was stirred at 50° C. for 30 min. A solution of 20.2 (17.2 g, 49.3 mmol) in anhydrous THF (100 mL) was added dropwise. After stirring at 50° C. for 16 h, the mixture was poured into saturated NH₄Cl (500 mL) and stirred for 10 min. The resultant suspension was extracted with EtOAc (2×200 mL) and the organic phase was washed with saturated brine, filtered and concentrated. The residue was recrystallized from MeOH: H₂O=1:1 (400 mL) to give the product 20.3 (16.0 g, 81.3%). ¹H NMR (400 MHz, CDCl3) δ_{H} 5.05-5.15 (m, 1H), 3.18-3.23 (m, 3H), 3.15 (s, 3H), 2.29-2.47 (m, 1H), 2.11-2.28 (m, 2H), 1.72-1.84 (m, 5H), 1.62-1.67 (m, 4H), 1.47-1.58 (m, 5H), 1.36-1.44 (m, 4H), 1.15-1.31 (m, 6H), 0.83-0.86 (m, 3H), 0.76-0.82 (m, 4H).

### Synthesis of 20.4

To a solution 20.3 (16.0 g, 44.3 mmol) in THF (200 ml) and was added HCl (44.3 ml, 2M, 88.6 mmol). The mixture was stirred at RT for 2 h and the pH of the mixture was adjusted to 7 with aqueous NaHCO₃. The suspension was extracted with EtOAc (2×100 mL) and the organic phase was washed with saturated brine, filtered and concentrated. The residue was purified by flash column (0-10% of EtOAc in PE) to give the product 20.4 (9.30 g, 60.6%). ¹H NMR (400 MHz, CDCl3)^{6H} 5.15-5.05 (m, 1H), 2.58-2.70 (m, 1H), 2.21-2.37 (m, 3H), 2.08-2.17 (m, 2H), 1.92-2.06 (m, 3H), 1.68-1.87 (m, 3H), 1.56-1.62 (m, 5H), 1.38-1.51 (m, 5H), 1.14-1.22 (m, 4H), 0.83 (s, 3H), 0.70-0.77 (m, 4H).

### Synthesis of 20.5

At −70° C., a cooled (−70° C.) LDA solution (3.5 mL, 2 M, 147 mmol) in THE was added to a stirred solution of 20.4 (9.30 g, 29.6 mmol) and ethyl diazoacetate (16.8 g, 147 mmol) in THE (450 mL). The mixture was stirred at −70° C. for another 2 h. HOAc (8.80 g, 147 mmol) in THE (50 mL) was added and the mixture was warmed to RT and stirred for 16 h. Water (1000 mL) and PE (300 mL) were added and the layers were separated. The aqueous phase was extracted with EtOAc (500 mL) and the combined organic layers were washed with saturated brine (1000 mL), dried over anhydrous Na₂SO₄ and filtered. Concentration of the filtrate afforded product 20.5 (15.5 g, crude), which was used directly to the next step.

### Synthesis of 20.6a &2 0.6b

To a solution of 20.5 (15.5 g, 15.1 mmol) in DME (250 mL) was added Rh₂(OAc)₄ (317 mg, 0.72 mmol). The reaction mixture was stirred at RT for 16 h then concentrated. The residue was purified by silica gel chromatography (0-20% of EtOAc in PE) to give a mixture of 20.6a & 20.6b (10.2 g, crude).

### Synthesis of 20.7a & 20.7b

To a mixture of 20.6a & 20.6b (10.2 g, 25.5 mmol) in MeOH (120 mL) was added H₂O (30 mL) and NaOH (8.11 g, 202.8 mmol). The mixture was stirred at 60° C. for 16 h, then cooled and concentrated. H₂O (500 mL) and EtOAc (500 mL) were added, and the layers were separated. The aqueous layer was extracted with EtOAc (500 mL) and the combined organic phase was washed with saturated brine (500 mL), dried over anhydrous Na₂SO₄, filtered and concentrated. The residue was purified by silica gel chromatography (0-5% of EtOAc in PE) to give a mixture of 20.7a & 20.7b (6.30 g, crude), which was further purified by SFC (Column: DAICEL CHIRALPAK AD (250 mm*50 mm, 10 μm); Condition: 0.1% NH₃H₂O EtOH)-ACN; Begin B: 20; End B: 20) to give the product 20.7a (1.80 g, 8.5%) and the product 20.7b (2.10 g, 10.1%).

20.7a: ¹H NMR (400 MHz, CDCl3) δ_{H} 5.15-4.98 (m, 1H), 2.50-2.01 (m, 8H), 1.83-1.75 (m, 1H), 1.70-1.65 (m, 3H), 1.61-1.32 (m, 6H), 1.30-0.85 (m, 11H), 0.82-0.69 (m, 6H).

20.7b: ¹H NMR (400 MHz, CDCl3) δ_{H} 5.25-5.00 (m, 1H), 3.35-3.15 (m, 1H), 2.45-2.10 (m, 5H), 1.95-1.85 (m, 1H), 1.80-1.75 (m, 4H), 1.70-1.35 (m, 9H), 1.30-1.20 (m, 9H), 0.90 (s, 3H), 0.81 (t, J=7.6 Hz, 3H).

### Synthesis of 20.8

At −70° C., to a freshly prepared MAD (19.1 mmol) solution in toluene (40 mL) was added 20.7b (2.10 g, 6.39 mmol) in DCM (20 mL) dropwise. After stirring at −70° C. for 1 h, MeMgBr (6.36 mL, 19.1 mmol) was added dropwise and the resulting solution was stirred at −70° C. for another 4 h. The reaction mixture was poured into saturated aqueous citric acid (200 mL), then extracted with EtOAc (2×50 mL). The combined organic layer was dried over Na₂SO₄, filtered and concentrated. The residue was purified by flash column (0-10% of EtOAc in PE) to give product 20.8 (2.00 g, crude). ¹H NMR (400 MHz, CDCl3) δ_{H} 5.15-5.05 (m, 1H), 3.41-3.37 (m, 1H), 2.55-2.23 (m, 5H), 1.92-1.68 (m, 6H), 1.44-1.39 (m, 12H), 0.94-0.83 (m, 14H).

### Synthesis of 20.9

To a solution of 20.8 (2.00 g, 5.80 mmol) in THE (20 mL) was added 9-BBN (2.83 mL, 11.6 mmol) and the mixture was stirred at 45° C. for 1 h. The mixture was cooled to 15° C. and ethanol (3.99 g, 86.9 mmol) was added, followed by NaOH aqueous solution (17.3 mL, 5.0 M, 86.9 mmol). H₂O₂ (8.69 mL, 10 M, 86.9 mmol) was added dropwise, and the reaction mixture was heated to 78° C. and stirred for 1 h. The mixture was cooled to 15° C. and poured into water (100 mL), then extracted with EtOAc (2×100 mL). The combined organic layer was washed with saturated brine (2×200 mL), drive over anhydrous Na₂SO₄, filtered and concentrated to give 20.9 (1.10 g, 52.3%). ¹H NMR (400 MHz, CDCl₃) δ_{H} 3.90-3.79 (m, 1H), 3.74-3.64 (m, 1H), 2.41-2.29 (m, 1H), 2.03-1.74 (m, 6H), 1.73-1.58 (m, 8H), 1.52-1.26 (m, 12H), 1.17-1.00 (m, 6H), 0.94-0.89 (m, 4H), 0.65 (s, 3H).

### Synthesis of 20.10

To a solution of 20.9 (1.10 g, 3.03 mmol) in DCM (20 ml), was added PCC (1.30 g, 1.21 mmol) and silica gel (1.30 g). The mixture was stirred at RT for 1 h then concentrated. The residue was purified by flash column (0-30% of EtOAc in PE) to give product 20.10 (800 mg, 73.3%). ¹H NMR (400 MHz, CDCl₃) S_{H} 2.57-2.46 (m, 1H), 2.41-2.31 (m, 1H), 2.10 (s, 3H), 2.06-1.93 (m, 2H), 1.88-1.80 (m, 1H), 1.76-1.60 (m, 6H), 1.55-1.33 (m, 7H), 1.32-0.99 (m, 12H), 0.91 (t, J=7.6 Hz, 4H), 0.59 (s, 3H).

### Synthesis of 20.11

At 0° C., to a solution of 20.10 (200 mg, 0.533 mmol) and HBr (21.1 mg, 0.106 mmol, 40%) in MeOH (10 mL) was added Br₂ (93.7 mg, 0.586 mmol). The mixture was stirred at RT for 2 h then poured into saturated NaHCO₃ (50 mL). The suspension was extracted with EtOAc (3×20 mL) and the combined organic layer was washed with saturated brine (50 mL), dried over anhydrous Na₂SO₄, filtered and concentrated to give 20.11 (150 mg, crude). ¹H NMR (400 MHz, CDCl₃) δ 3.97-3.80 (m, 2H), 2.86-2.77 (m, 1H), 2.44-2.33 (m, 1H), 2.23-2.11 (m, 1H), 2.00-1.82 (m, 3H), 1.78-1.61 (m, 6H), 1.54-1.28 (m, 8H), 1.27-1.00 (m, 12H), 0.98-0.89 (m, 4H), 0.63 (s, 3H).

### Synthesis of 20

To a solution of 20.11 (150 mg, 0.33 mmol) in acetone (5 ml) was added K₂CO₃ (91.0 mg, 0.66 mmol) and 1H-pyrazole-4-carbonitrile (36.8 mg, 0.39 mmol), and the mixture was stirred at RT for 2 h. The reaction mixture was poured into water and extracted with EtOAc (2×20 ml). The combined organic phase was dried over anhydrous Na₂SO₄, filtered and concentrated. The residue was purified by flash column (0-30% of EtOAc in PE) to give product 20 (100 mg, crude), which was further purified by HPLC (Column: YMC Triart C18 150*25 mm*5 μm); condition: water (10 mM NH₄HCO₃)-ACN; Begin B: 62%; End B: 69%) to afford 20 (62.3 mg, 62.4%). ¹H NMR (400 MHz, CDCl₃) δ_{H} 7.85 (s, 1H), 7.81 (s, 1H), 5.07-4.84 (m, 2H), 2.63-2.52 (m, 1H), 2.43-2.36 (m, 1H), 2.24-2.14 (m, 1H), 2.07-1.92 (m, 2H), 1.89-1.82 (m, 1H), 1.77-1.65 (m, 6H), 1.54-1.32 (m, 7H), 1.30-1.07 (m, 11H), 0.92 (s, 4H), 0.66 (s, 3H). LC-ELSD/MS: purity>99%, MS ESI calcd. for C₂₈H₄₂N₃O₂ [M+H]⁺ 452.3, found 452.3.

### Example 21 & 22 & 23 & 24: Synthesis of 1-(2-((2R,4aS,4bS,6aS,7R,11aS,11bS,13aR)-4a-ethyl-2-hydroxy-2,6a-dimethyloctadecahydro-1H-cyclohepta[alphenanthren-7-yl)-2-oxoethyl)-1H-pyrazole-4-carbonitrile (21) & Synthesis of 1-((2R,4aS,4bS,6aS,7S,11aS,11bS,13aR)-4a-ethyl-2-hydroxy-2,6a-dimethyloctadecahydro-1H-cyclohepta[alphenanthren-7-yl)ethanone (22) & Synthesis of 1-((2R,4aS,4bS,6aS,7R,11aS,11bS,13aR)-4a-ethyl-2-hydroxy-2,6a-dimethyloctadecahydro-1H-cyclohepta[a]phenanthren-7-yl)ethanone (23) & Synthesis of 1-(2-((2R,4aS,4bS,6aS,7S,11aS,11bS,13aR)-4a-ethyl-2-hydroxy-2,6a-dimethyloctadecahydro-1H-cyclohepta[alphenanthren-7-yl)-2-oxoethyl)-1H-pyrazole-4-carbonitrile (24)

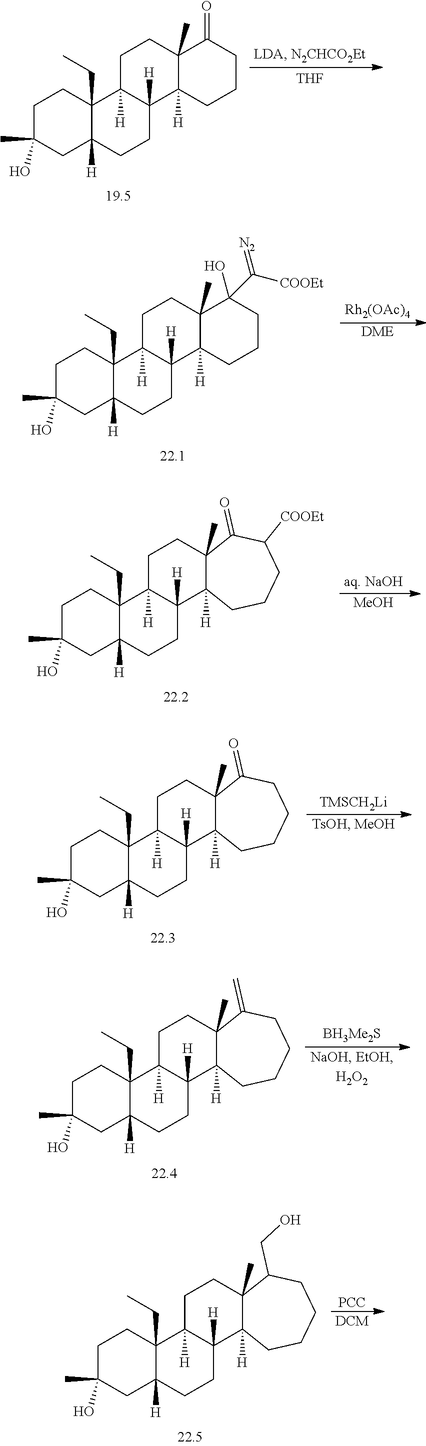

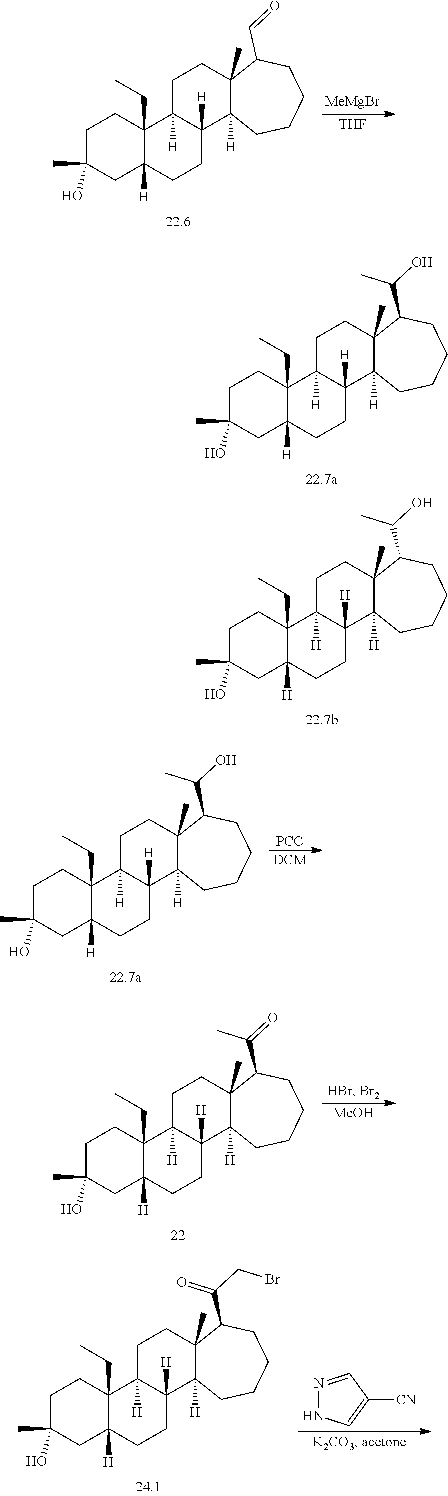

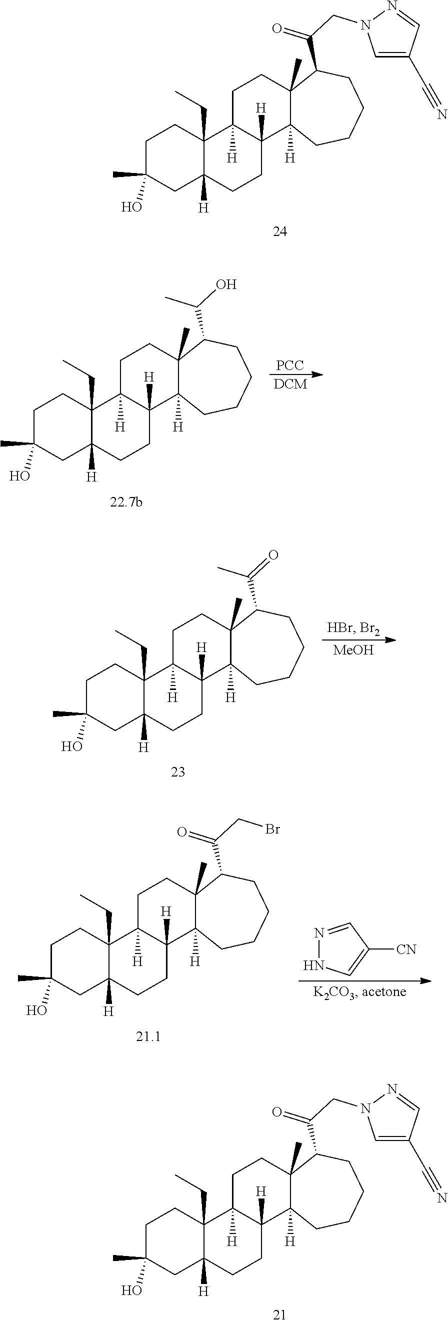

### Synthesis of 22.1

At −70° C., a cooled (−70° C.) LDA solution (37.5 mL, 2M, 75.0 mmol) in THF was added to a stirred solution of 19.5 (5.00 g, 15.0 mmol) and ethyl diazoacetate (8.55 g, 75.0 mmol) in THF (120 mL). After stirring at −70° C. for 2 h, HOAc (4.50 g, 75.0 mmol) in THF (30 mL) was added and the mixture was warmed to RT and stirred for another 16 h. Water (300 mL) and PE (200 mL) was added and the layers were separated. The aqueous phase was extracted with EtOAc (200 mL) and the combined organic layers were washed with saturated brine (600 mL), dried over anhydrous Na₂SO₄, filtered and concentrated. The residue was purified by flash column (0˜35% of EtOAc in PE) to give product 22.1 (3.50 g, impure).

### Synthesis of 22.2

To a solution of 22.1 (3.50 g, 7.83 mmol) in DME (100 mL) was added Rh₂(OAc)₄ (69.2 mg, 0.16 mmol). The reaction mixture was stirred at 25° C. for 16 h, then concentrated to give product 22.2 (3.50 g, crude).

### Synthesis of 22.3

To a solution of 22.2 (3.30 g, 7.88 mmol) in MeOH (100 mL) was added H₂O (30 mL) and NaOH (4.72 g, 118 mmol). The mixture was stirred at 80° C. for 16 h then concentrated. H₂O (150 mL) was added and the mixture was extracted with EtOAc (2×150 mL). The combined organic phase was washed with saturated brine (300 mL), dried over anhydrous Na₂SO₄, filtered and concentrated to give the product (1.10 g, crude). 50 mg crude product 22.3 was purified by silica gel chromatography (0-30% of EtOAc in PE) to give the pure 22.3 (18.5 mg, 37.0%). ¹H NMR (400 MHz, CDCl₃)^{6H} 3.12-2.97 (m, 1H), 2.33-2.21 (m, 1H), 2.07-1.94 (m, 1H), 1.85-1.59 (m, 10H), 1.52-1.34 (m, 10H), 1.31-1.19 (m, 7H), 1.14-1.01 (m, 5H), 0.79 (t, J=7.6 Hz, 3H). LC-ELSD/MS: purity>99%, MS ESI calcd. for C₂₃H₃₇O [M−H₂O+H]⁺329.3, found 329.3.

### Synthesis of 22.4

At −40° C., to a solution of TMSCH₂Li (51.2 mL, 28.7 mmol, 0.56 M) in THF (20 mL) was added a solution of 22.3 (1.00 g, 2.88 mmol) in THF (20 mL). The reaction was warmed to RT and stirred for 16 h. Saturated NH₄Cl (100 mL) was added and the suspension was extracted with EtOAc (2×60 mL). The combined organic layer was washed with saturated brine (100 mL), dried over anhydrous Na₂SO₄, filtered and concentrated. The residue was dissolved in MeOH (50 mL), and p-TsOH (50 mg) was added. The mixture was stirred for 10 min and poured into saturated NaHCO₃ (100 mL). The resultant suspension was extracted with EtOAc (3×50 mL) and the combined organic layer was washed with saturated brine (100 mL), dried over anhydrous Na₂SO₄, filtered and concentrated. The residue was purified by flash column (0-25% of EtOAc in PE) to give 22.4 (1.00 g, crude). ¹H NMR (400 MHz, CDCl₃)^{6H} 4.79 (s, 1H), 4.72 (s, 1H), 2.49-2.37 (m, 1H), 2.24-2.13 (m, 1H), 2.03-1.90 (m, 1H), 1.81-1.50 (m, 11H), 1.48-1.30 (m, 10H), 1.25-1.12 (m, 6H), 1.08-0.94 (m, 5H), 0.78 (t, J=7.6 Hz, 3H).

### Synthesis of 22.5

To a solution of 22.4 (1.00 g, 2.90 mmol) in THE (15 mL) was added BH₃-Me₂S (1.44 mL, 10 M, 14.4 mmol) and the mixture was stirred at 45° C. for 1 h. The mixture was cooled to 0° C. and ethanol (2.66 g, 57.9 mmol) was added, followed by NaOH (11.5 mL, 5.0 M, 57.9 mmol). H₂O₂(5.79 mL, 10 M, 57.9 mmol) was added dropwise and the reaction mixture was heated to 70° C. for 1 h. The mixture was cooled, poured into water (100 mL) and extracted with EtOAc (2×50 mL). The combined organic layer was washed with saturated brine (2×100 mL), drive over anhydrous Na₂SO₄, filtered and concentrated to give 22.5 (1.20 g, crude).

### Synthesis of 22.6

To a solution of 22.5 (550 mg, crude) in DCM (10 mL) was added silica gel (1.50 g) and PCC (649 mg, 3.02 mmol), and the mixture was stirred at RT for 1 h. The suspension was filtered, and the filter cake was washed with DCM (3×50 mL). The filtrate was concentrated to give 22.6 (500 mg, crude), which was used to next step directly.

### Synthesis of 22.7a & 22.7b

At 0° C., to a solution of 22.6 (500 mg, crude) in THF (10 mL) was added MeMgBr (2.39 mL, 7.19 mmol, 3M). The reaction mixture was warmed to RT and stirred for 1 h. Saturated NH₄Cl (50 mL) solution was added and the suspension was extracted with EtOAc (3×50 mL). The combined organic layer was washed with saturated brine (100 mL), dried over anhydrous Na₂SO₄, filtered and concentrated. The residue was purified by flash column (0-40% of EtOAc in PE) to give 22.7a (100 mg, 19.2%) and 22.7b (100 mg, 19.2%).

22.7a: ¹H NMR (400 MHz, CDCl₃) S_{H} 4.33-4.18 (m, 1H), 2.01-1.92 (m, 2H), 1.89-1.59 (m, 6H), 1.50-1.30 (m, 12H), 1.29-1.21 (m, 6H), 1.21-1.07 (m, 6H), 1.05-0.94 (m, 2H), 0.92-0.86 (m, 4H), 0.85-0.74 (m, 5H).

22.7b: ¹H NMR (400 MHz, CDCl₃) δ_{H}4.15-4.05 (m, 1H), 1.84-1.70 (m, 4H), 1.66-1.52 (m, 10H), 1.49-1.33 (m, 8H), 1.32-1.26 (m, 3H), 1.24-1.17 (m, 6H), 1.15-0.90 (m, 6H), 0.85-0.81 (m, 3H), 0.78 (t, J=7.6 Hz, 3H).

### Synthesis of 22

To a solution of 22.7a (150 mg, 0.40 mmol) in DCM (10 mL) was added silica gel (200 mg) and PCC (171 mg, 0.80 mmol). The reaction mixture was stirred at RT for 1 h to give a yellow suspension. The suspension was filtered, and the filter cake was washed with DCM (3×20 mL). The filtrate was concentrated, and the residue was purified by flash column (0-10% of EtOAc in PE) to give the crude product 22 (130 mg), which was further purified by flash column (0˜35% of EtOAc in PE) to give product 22 (15 mg, 10.1%). 1H NMR (400 MHz, CDCl3) δ_{H} 2.42-2.36 (m, 1H), 2.15-2.11 (m, 3H), 1.95 (t, J=12.4 Hz, 1H), 1.81-1.61 (m, 8H), 1.56-1.51 (m, 6H), 1.46-1.32 (m, 6H), 1.29-1.21 (m, 7H), 1.18-1.08 (m, 3H), 1.02-0.94 (m, 4H), 0.76 (t, J=7.6 Hz, 3H). LC-ELSD/MS: purity>99%, MS ESI calcd. for C₂₅H₄₁O [M−H₂O+H]⁺ 357.3, found 357.3.

### Synthesis of 24.1

At 0° C., to a solution of 22 (60.0 mg, 0.16 mmol) and HBr (1.59 mg, 0.01 mmol, 40%) in MeOH (5 mL) was added Br₂ (28.1 g, 0.18 mmol), and the mixture was stirred at RT for 2 h. Saturated NaHCO₃ (20 mL) was added and the suspension was extracted with EtOAc (3×20 mL). The combined organic layer was washed with saturated brine (30 mL), dried over anhydrous Na₂SO₄, filtered and concentrated to give the product 24.1 (71.0 mg, crude), which was used directly to next step.

### Synthesis of 24

To a solution of 24.1 (70.0 mg, 0.15 mmol) in acetone (5 mL) was added K₂CO₃ (42.5 mg, 0.31 mmol) and 1H-pyrazole-4-carbonitrile (17.2 mg, 0.18 mmol). The mixture was stirred at RT for 2 h to give a yellow suspension. The reaction was quenched with saturated aqueous NH₄Cl solution (30 mL) and extracted with EtOAc (2×30 mL). The combined organic phase was washed with saturated brine (50 mL), dried over anhydrous Na₂SO₄, filtered and concentrated to give the crude product, which was purified by flash column (0-70% of EtOAc in PE) to give 24 (21.6 mg, 30.1%). ¹H NMR (400 MHz, CDCl₃) δ_{H} 7.82 (s, 2H), 4.99 (s, 2H), 2.44-2.37 (m, 1H), 1.99-1.91 (m, 1H), 1.87-1.70 (m, 5H), 1.70-1.61 (m, 4H), 1.53-1.35 (m, 8H), 1.35-1.28 (m, 2H), 1.26 (s, 3H), 1.25-1.03 (m, 7H), 1.01 (s, 3H), 1.01-0.91 (m, 2H), 0.77 (t, J=7.40 Hz, 3H). LC-ELSD/MS: purity>99%, MS ESI calcd. for C₂₉H₄₂N₃[M−H₂O+H]⁺ 448.3, found 448.3.

### Synthesis of 23

To a solution of 22.7b (130 mg, 0.34 mmol) in DCM (10 mL) was added silica gel (200 mg) and PCC (148 mg, 0.69 mmol). The reaction mixture was stirred at RT for 2 h to give a yellow suspension. The mixture was filtered, and the filter cake was washed with DCM (3×20 mL). The filtrate was concentrated, and the residue was purified by flash column (0-40% of EtOAc in PE) to give crude product 23 (100 mg, 77.5%). 50.0 mg of crude 23 was purified by flash column (0-35% of EtOAc in PE) to give the product 23 (15.0 mg). ¹H NMR (400 MHz, CDCl₃) OH 2.40-2.38 (m, 1H), 2.12-2.08 (m, 3H), 2.03-1.56 (m, 11H), 1.55-1.27 (m, 11H), 1.26-1.11 (m, 11H), 1.09-1.00 (m, 4H), 0.77 (s, 3H). LC-ELSD/MS: purity>99%, MS ESI calcd. for C₂₅H₄₁O [M−H₂O+H]⁻ 357.3, found 357.3.

### Synthesis of 21.1

At 0° C., to a solution of 23 (40.0 mg, 0.11 mmol) and HBr (1.06 mg, 0.01 mmol, 40%) in MeOH (5 mL) was added Br₂ (18.7 mg, 0.12 mmol). The mixture was stirred at RT for 2 h, then quenched with saturated NaHCO₃ (20 mL). The suspension was extracted with EtOAc (3×20 mL). The combined organic layer was washed with saturated brine (30 mL), dried over anhydrous Na₂SO₄, filtered and concentrated to give product 21.1 (45.0 mg, crude), which was used directly to the next step.

### Synthesis of 21

To a solution of 21.1 (40.0 mg, 0.09 mmol) in acetone (5 mL) was added K₂CO₃ (24.3 mg, 0.17 mmol) and 1H-pyrazole-4-carbonitrile (9.80 mg, 0.11 mmol), and the mixture was stirred at RT for 2 h. The reaction was quenched with saturated aqueous NH₄Cl solution (30 mL) and extracted with EtOAc (2×30 mL). The combined organic phase was washed with saturated brine (50 mL), dried over anhydrous Na₂SO₄, filtered and concentrated. The residue was purified by silica gel chromatography (0-70% of EtOAc in PE) to give the pure product 21 (31.8 mg, 75.6%). ¹H NMR (400 MHz, CDCl₃) S_{H} 7.85 (s, 1H), 7.80 (s, 1H), 5.09-4.90 (m, 2H), 2.59 (d, J=9.6 Hz, 1H), 2.00-1.86 (m, 3H), 1.79-1.62 (m, 7H), 1.52-1.36 (m, 9H), 1.30-1.20 (m, 10H), 1.15-1.03 (m, 6H), 0.78 (t, J=7.6 Hz, 3H). LC-ELSD/MS: purity>99%, MS ESI calcd. for C₂₉H₄₂N₃O [M−H₂O+H]⁺448.3, found 448.3.

### Example 25 & 26: Synthesis of 1-((1S,3aS,3bS,5aR,8S,10aS,10bS,12aS)-10a-ethyl-8-hydroxy-8,12a-dimethyloctadecahydrocyclohepta[a]cyclopenta[f]naphthalen-1-yl)ethanone (25) & Synthesis of 1-(2-((1S,3aS,3bS,5aR,8S,10aS,10bS,12aS)-10a-ethyl-8-hydroxy-8,12a-dimethyloctadecahydrocyclohepta[ajcyclopenta[f]naphthalen-1-yl)-2-oxoethyl)-1H-pyrazole-4-carbonitrile (26)

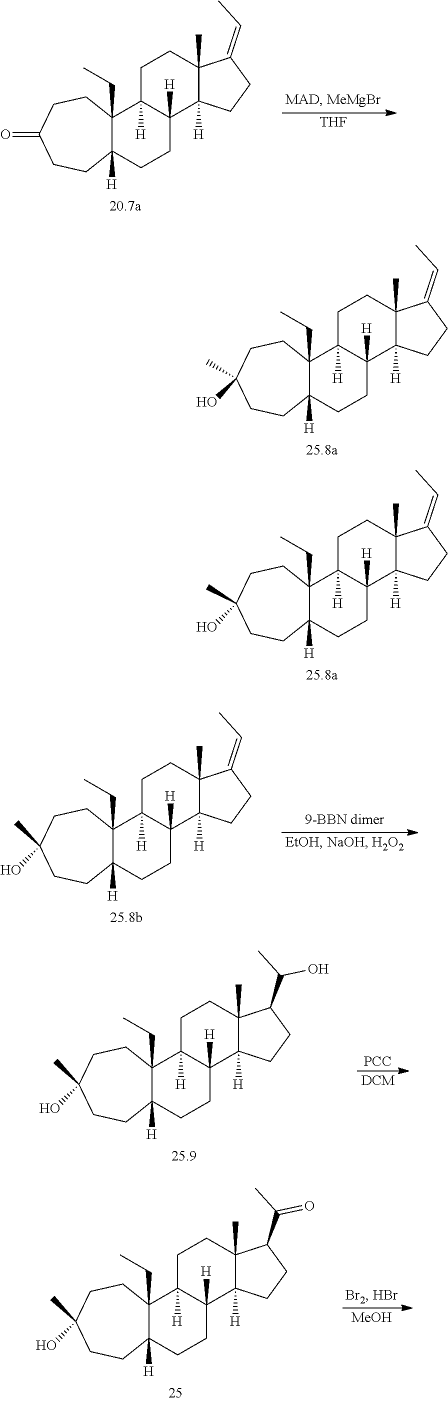

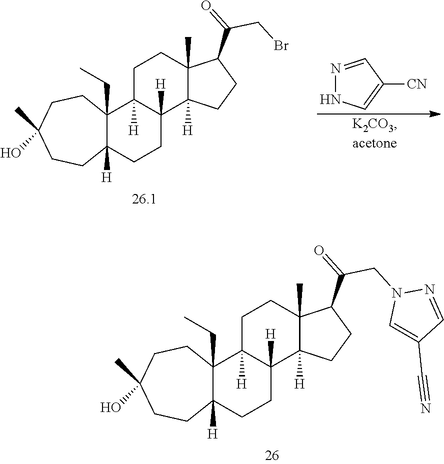

### Synthesis of 25.8a & 25.8b

A freshly prepared MAD (16.4 mmol) solution was cooled to −70° C. and 20.7a (1.80 g, 5.47 mmol) in DCM (20 mL) was added dropwise. After stirring at −70° C. for 1 h, MeMgBr (5.46 mL, 16.4 mmol) was added dropwise at −70° C. The resulting solution was stirred at −70° C. for another 4 h, then poured into saturated aqueous citric acid (200 mL). The suspension was extracted with EtOAc (2×50 mL) and the combined organic layer was dried over Na₂SO₄, filtered and concentrated. The residue was purified by flash column (0-30% of EtOAc in PE) to give products 25.8a (460 mg, 24.4%) and 25.8b (590 mg, 31.3%).

25.8a: ¹H NMR (400 MHz, CDCl₃) δ_{H} 5.15-5.06 (m, 1H), 2.43-2.31 (m, 1H), 2.28-2.09 (m, 2H), 1.88-1.72 (m, 2H), 1.72-1.57 (m, 10H), 1.55-1.47 (m, 2H), 1.47-1.33 (m, 5H), 1.33-1.22 (m, 5H), 1.20 (s, 3H), 1.19-1.05 (m, 2H), 1.02-0.89 (m, 1H), 0.88-0.79 (m, 6H).

25.8b: ¹H NMR (400 MHz, CDCl₃)⁶H 5.18-5.03 (m, 1H), 2.40-2.29 (m, 1H), 2.27-2.11 (m, 2H), 2.01-1.85 (m, 1H), 1.78-1.68 (m, 2H), 1.66-1.60 (m, 7H), 1.55-1.47 (m, 3H), 1.46-1.35 (m, 6H), 1.35-1.25 (m, 3H), 1.20 (s, 3H), 1.19-1.04 (m, 4H), 1.03-0.92 (m, 1H), 0.88-0.80 (m, 6H).

### Synthesis of 25.9

At 15° C., to a solution of 25.8b (590 mg, 1.71 mmol) in THF (10 mL) was added 9-BBN dimer (1.25 mg, 5.13 mmol) and the mixture was stirred at 40° C. for 1 h. The resulting mixture was cooled to 15° C. and ethanol (787 mg, 17.1 mmol) was added, followed by NaOH aqueous solution (3.42 mL, 5M, 17.1 mmol). The mixture was cooled to −10° C., H₂O₂(1.71 mL, 10 M, 17.1 mmol) was added dropwise and the mixture was heated at 80° C. for 1 h. After cooling, saturated Na₂S₂O₃ (50 mL) was added and the mixture was extracted with EtOAc (100 mL). The combined organic phase was washed with saturated brine (2×100 mL), dried over anhydrous Na₂SO₄ and concentrated to give 25.9 (900 mg, crude). 1H NMR (400 MHz, CDCl₃)⁶H 3.77-3.64 (m, 1H), 1.87-1.78 (m, 5H), 1.72-1.65 (m, 5H), 1.55-1.44 (m, 11H), 1.38-1.32 (m, 6H), 1.20 (s, 3H), 1.17-0.96 (m, 6H), 0.84 (t, 0.1=7.60 Hz, 3H), 0.64 (s, 3H).

### Synthesis of 25

At 0° C., to a solution of 25.9 (900 mg, 2.48 mmol) in DCM (10 mL) was added silica gel (2.00 g) and PCC (1.59 g, 7.44 mmol), and the mixture was stirred at RT for 1 h. The suspension was filtered, and the filter cake was washed with DCM (2×30 mL). The filtrate was concentrated and the residue was purified by flash column (0˜40% of EtOAc in PE) to give 25 (600 mg, impure), which was further purified by flash column (0˜30% of EtOAc in PE) to give 25 (360 mg, 72.4%). ¹H NMR (400 MHz, CDCl₃)⁶H 2.52 (t, J=8.40 Hz, 1H), 2.27-2.12 (m, 1H), 2.10 (s, 3H), 2.03-1.86 (m, 2H), 1.76-1.68 (m, 2H), 1.65-1.58 (m, 7H), 1.49-1.36 (m, 6H), 1.35-1.28 (m, 4H), 1.21 (s, 3H), 1.20-1.07 (m, 4H), 1.01-0.91 (m, 1H), 0.84 (t, J=7.60 Hz, 3H), 0.59 (s, 3H). LC-ELSD/MS: purity>99%, MS ESI calcd. for C₂₄H₃₉O [M−H₂O+H]⁺ 343.3, found 343.3.

### Synthesis of 26.1

At 0° C., to a solution of 25 (100 mg, 0.277 mmol) and HBr (5.52 mg, 0.028 mmol, 40%) in MeOH (5 mL) was added Br₂ (48.8 mg, 0.305 mmol). The mixture was stirred at RT for 2 h, then poured into saturated NaHCO₃ (50 mL). The suspension was extracted with EtOAc (3×20 mL). The combined organic layer was washed with saturated brine (50 mL), dried over anhydrous Na₂SO₄, filtered and concentrated to give 26.1 (100 mg, crude). ¹H NMR (400 MHz, CDCl₃)^{6H} 3.90 (d, J=3.60 Hz, 2H), 2.85-2.76 (m, 1H), 2.23-2.14 (m, 1H), 1.95-1.85 (m, 3H), 1.77-1.66 (m, 6H), 1.65-1.63 (m, 1H), 1.53-1.44 (m, 5H), 1.41-1.28 (m, 8H), 1.21 (s, 3H), 1.14-1.07 (m, 2H), 0.98-0.91 (m, 1H), 0.84 (t, J=7.60 Hz, 3H), 0.62 (s, 3H).

### Synthesis of 26

To a solution of 26.1 (100 mg, 0.2275 mmol) in acetone (5 ml) was added K₂CO₃ (62.7 mg, 0.455 mmol) and 1H-pyrazole-4-carbonitrile (25.4 mg, 0.2729 mmol), and the mixture was stirred at RT for 2 h. The mixture was poured into water (50 mL) then extracted with EtOAc (2×50 mL). The combined organic phase was washed with saturated brine, filtered and concentrated. The residue was purified by flash column (0-70% of EtOAc in PE) to give 26 (35.3 mg, 34.6%). ¹H NMR (400 MHz, CDCl₃)^{6H} 7.85 (s, 1H), 7.81 (s, 1H), 5.04-4.97 (m, 1H), 4.92-4.85 (m, 1H), 2.64-2.54 (m, 1H), 2.26-2.12 (m, 1H), 2.10-2.00 (m, 1H), 1.99-1.87 (m, 1H), 1.76-1.60 (m, 8H), 1.54-1.23 (m, 13H), 1.22 (s, 3H), 1.16-1.06 (m, 2H), 1.05-0.90 (m, 1H), 0.85 (t, J=7.20 Hz, 3H), 0.65 (s, 3H). LC-ELSD/MS: purity>99%, MS ESI calcd. for C₂₈H₄₀N₃O [M−H₂O+H]⁺ 434.3, found 434.3.

### Example 27 & 28: Synthesis of 1-((1S,4aS,4bR,6aR,8R,10aS,10bR,12aS)-12a-ethyl-8-hydroxy-8-methyloctadecahydrochrysen-1-yl)ethanone (27) & Synthesis of 1-(2-((1S,4aS,4bR,6aR,8R,10aS,10bR,12aS)-12a-ethyl-8-hydroxy-8-methyloctadecahydrochrysen-1-yl)-2-oxoethyl)-1H-pyrazole-4-carbonitrile (28)

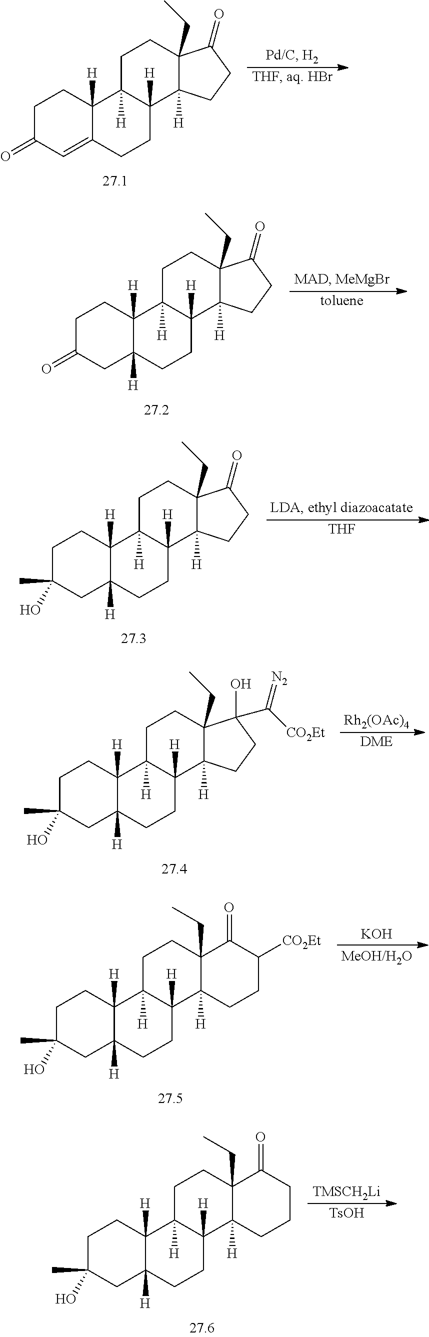

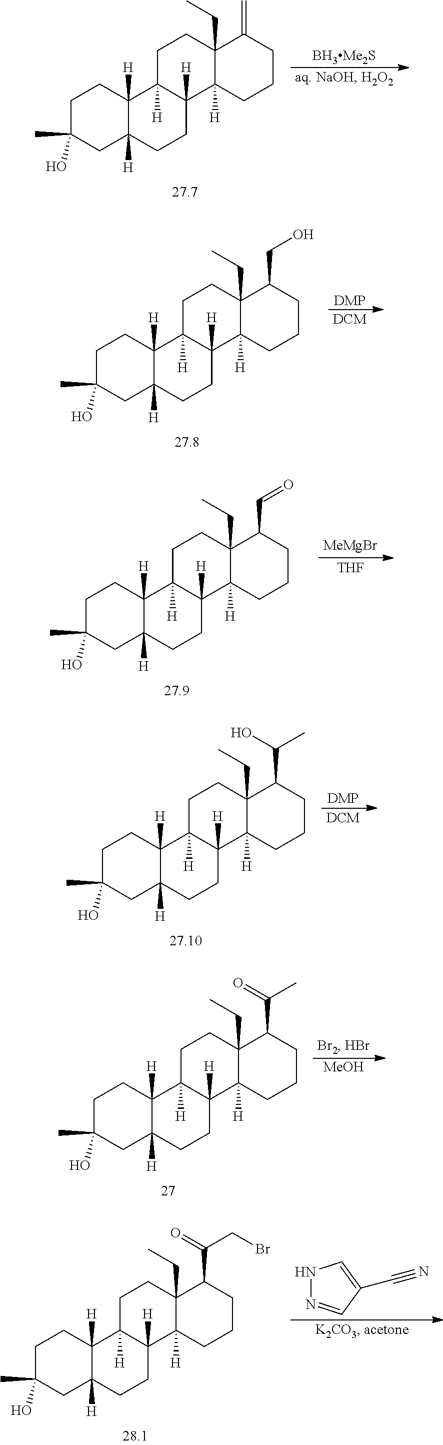

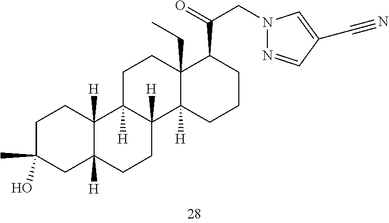

### Synthesis of 27.2

To a solution of 27.1 (10.0 g, 34.9 mmol) and Pd/C (1.00 g, 10%, 50% water wet) in THF (100 mL) was added HBr (0.5 mL, 40% in water). The mixture was hydrogenated under 15 psi of hydrogen at RT for 12 h. The reaction mixture was filtered through a pad of Celite and washed with EtOAc (3×50 mL). The filtrate was concentrated to afford the crude product, which was triturated from PE (100 mL) to give 27.2 (10.0 g, crude). ¹H NMR (400 MHz, CDCl₃) S_{H} 2.58 (t, J=14.0, 1H), 2.50-2.35 (m, 1H), 2.30-2.00 (m, 7H), 1.99-1.88 (m, 1H), 1.87-1.59 (m, 6H), 1.52-1.08 (m, 9H), 0.79 (t, J=7.2 Hz, 3H).

### Synthesis of 27.3

A freshly prepared MAD (103 mmol) solution (see synthesis 19.6) was cooled to −70° C. and a solution of 27.2 (10.0 g, 34.6 mmol) in anhydrous DCM (100 mL) was added dropwise. After stirring at −70° C. for 1 h, MeMgBr (34.3 mL, 103 mmol, 3M in ethyl ether) was added dropwise, and the resulting solution was stirred at −70° C. for another 2 h. The reaction mixture was poured into saturated citric acid (500 mL) then extracted with EtOAc (3×200 mL). The combined organic layer was washed with saturated brine (500 mL), dried over anhydrous Na₂SO₄, filtered and concentrated. The residue was purified by flash column (0-20% of EtOAc in PE) to give 27.3 (10.0 g, 95%). ¹H NMR (400 MHz, CDCl₃) S_{H} 2.45-2.35 (m, 1H), 2.15-2.05 (m, 1H), 1.95-1.59 (m, 9H), 1.52-1.28 (m, 8H), 1.25-0.80 (m, 10H), 0.76 (t, J=7.2 Hz, 3H).

### Synthesis of 27.4

A solution of LDA (81.5 mL, 2 M, 163 mmol) was cooled to −70° C., then added to a cooled (−70° C.) and stirred solution of 27.3 (10.0 g, 32.8 mmol) and ethyl diazoacetate (18.5 g, 163 mmol) in THF (200 mL). The mixture was stirred at −70° C. for 4 h. HOAc (9.80 g, 163 mmol) in THF (20 mL) was added and the mixture was warmed to RT then stirred for 16 h. Water (500 mL) was added and the mixture was extracted with EtOAc (3×200 mL). The combined organic layers were washed with saturated brine (500 mL), dried over anhydrous Na₂SO₄, and evaporated to give the crude product, which was then purified by CombiFlash (0-30% of EtOAc in PE) to give 27.4 (4.90 g). ¹H NMR (400 MHz, CDCl₃) δ_{H} 4.32-4.20 (m, 2H), 2.30-2.15 (m, 1H), 1.98-1.59 (m, 8H), 1.52-1.31 (m, 9H), 1.30-1.11 (m, 10H), 1.10-0.75 (m, 8H).

### Synthesis of 27.5

A solution of 27.4 (6.00 g, 14.3 mmol) and Rh₂(OAc)₄ (100 mg) in DME (100 mL) was stirred at RT for 16 h. The mixture was poured into saturated brine (100 mL) then extracted with EtOAc (2×100 mL). The combined organic layer was washed with saturated brine (200 mL), dried over anhydrous Na₂SO₄, filtered and concentrated to give 27.5 (5.50 g, crude), which was used for the next step directly.

### Synthesis of 27.6

A solution of 27.5 (5.50 g) and KOH (4.70 g, 84.0 mmol) in MeOH (60 mL) was stirred at 70° C. for 2 h. After cooled to RT, the mixture was poured into water (100 mL) then extracted with (3×50 mL). The combined organic layer was washed with saturated brine (200 mL), dried over anhydrous Na₂SO₄, filtered and concentrated to give 27.6 (2.00 g, 45%). ¹H NMR (400 MHz, CDCl₃) δ_{H} 2.58-2.45 (m, 1H), 2.25-2.15 (m, 1H), 2.14-2.05 (m, 1H), 2.00-1.65 (m, 10H), 1.53-1.36 (m, 5H), 1.35-1.20 (m, 10H), 1.10-0.82 (m, 3H), 0.65 (t, J=7.6 Hz, 3H).

### Synthesis of 27.7

At −30° C., to a solution of 27.6 (1.00 g, 3.1 mmol) in THF (5 mL) was added TMSCH₂Li (44.6 mL, 25.0 mmol, 0.56 M) and the mixture was warmed to RT and stirred for 16 h. The mixture was poured into saturated NH₄Cl (100 mL) then extracted with EtOAc (3×50 mL). The combined organic layer was washed with saturated brine (100 mL), dried over anhydrous Na₂SO₄, filtered and concentrated. The residue was dissolved in MeOH (20 mL) and p-TsOH (100 mg) was added. The mixture was stirred at RT for 30 minutes then poured into saturated NaHCO₃ (100 mL). The suspension was extracted with EtOAc (3×50 mL) and the combined organic layer was washed with saturated brine (100 mL), dried over anhydrous Na₂SO₄, filtered and concentrated. The residue was purified by flash column (0-15% of EtOAc in PE) to give 27.7 (300 mg, 30%). ¹H NMR (400 MHz, CDCl₃)^{6H} 4.72 (s, 1H), 4.52 (s, 1H), 2.35-2.00 (m, 2H), 1.99-1.59 (m, 8H), 1.52-1.28 (m, 9H), 1.26 (s, 3H), 1.25-0.82 (m, 9H), 0.58 (t, J=7.6 Hz, 3H).

### Synthesis of 27.8

At 0° C., to a solution of 27.7 (600 mg, 1.9 mmol) in THF (5 mL) was added BH₃-Me₂S (1.0 mL, 10 M, 10.0 mmol) and the mixture was stirred at 25° C. for 16 h. EtOH (20 mL) was added and the mixture was cooled to −10° C. NaOH aqueous (4.0 mL, 5 M, 20.0 mmol) solution was added, followed by H₂O₂ (2.30 g, 20.0 mmol, 30% in water). After the addition, the mixture was heated to 70° C. and stirred for 1 h. The mixture was cooled to RT and extracted with EtOAc (3×50 mL). The combined organic layer was washed with saturated Na₂S₂O₃ (200 mL), saturated brine (200 mL), dried over anhydrous Na₂SO₄, filtered and concentrated to give 27.8 (400 mg, crude). ¹H NMR (400 MHz, CDCl₃) δ_{H} 3.95-3.85 (m, 1H), 3.80-3.68 (m, 1H), 2.18-2.08 (m, 1H), 1.95-1.63 (m, 10H), 1.50-1.35 (m, 5H), 1.32-1.28 (m, 5H), 1.05-0.80 (m, 12H), 0.74 (t, J=7.6 Hz, 3H).

### Synthesis of 27.9

A solution of 27.8 (400 mg, crude) and Dess Martin Reagent (1.00 g, 2.4 mmol) in DCM (20 mL) was stirred at RT for 30 min. Saturated NaHCO₃ (100 mL) was added and the suspension was extracted with DCM (2×50 mL). The combined organic layer was washed with saturated Na₂S₂O₃ (2×100 mL), saturated brine (100 mL), dried over anhydrous Na₂SO₄, filtered and concentrated to give 27.9 (300 mg, crude). ¹H NMR (400 MHz, CDCl₃) δH 9.98 (s, 1H), 2.40-2.01 (m, 4H), 1.92-1.72 (m, 8H), 1.48-1.32 (m, 11H), 1.01-0.81 (m, 9H), 0.70 (t, J=7.6 Hz, 3H).

### Synthesis of 27.10

At 0° C., to a solution of 27.9 (300 mg, crude) in THF (5 mL) was added MeMgBr (1.5 mL, 4.5 mmol, 3M) and the mixture was stirred at RT for 1 h. The mixture was poured into saturated NH₄Cl (100 mL) and the suspension was extracted with EtOAc (3×20 mL). The combined organic layer was washed with saturated brine (100 mL), dried over anhydrous Na₂SO₄, filtered and concentrated. The residue was purified by flash column (0-10% of EtOAc in PE) to give 27.10 (150 mg, 48%). ¹H NMR (400 MHz, CDCl₃) S_{H} 4.30-4.20 (m, 1H), 2.25-2.15 (m, 1H), 1.95-1.62 (m, 6H), 1.50-1.25 (m, 14H), 1.24-1.01 (m, 7H), 1.00-0.66 (m, 11H).

### Synthesis of 27

A solution of 27.10 (150 mg, 0.43 mmol) and Dess Martin Reagent (364 mg, 0.86 mmol) in DCM (5 mL) was stirred at RT for 30 min. The mixture was poured into saturated NaHCO₃ (100 mL) and the resultant suspension was extracted with DCM (3×20 mL). The combined organic layer was washed with saturated Na₂S₂O₃ (2×100 mL), saturated brine (100 mL), dried over anhydrous Na₂SO₄, filtered and concentrated to give 27 (100 mg, 67%). 27 (10.0 mg) was purified by pre-HPLC (Column: Xtimate C18 150*25 mm*5 μm, Condition: water (10 mM NH₄HCO₃)-ACN, Begin B: 75, End B: 95, Gradient Time (min): 7) to give pure 27 (2.00 mg, 20%). ¹H NMR (400 MHz, CDCl₃)⁶H 2.20-2.15 (m, 4H), 2.10-1.99 (m, 1H), 1.95-1.59 (m, 9H), 1.52-1.25 (m, 14H), 1.24-0.78 (m, 7H), 0.69 (t, J=7.6 Hz, 3H). LC-ELSD/MS: purity≥99%, MS ESI calcd. for C₂₃H₃₇O [M−H₂O+H]⁺329.3, found 329.3.

### Synthesis of 28.1

At 0° C., to a solution of 27 (90.0 mg, 0.26 mmol) in MeOH (5.0 mL) was added HBr (10.3 mg, 0.05 mmol, 40%) and Br₂ (41.5 mg, 0.26 mmol) and the reaction mixture was stirred at RT for 16 h. The mixture was poured into saturated NaHCO₃ (50 mL) and the suspension was extracted with EtOAc (3×20 mL). The combined organic layer was washed with saturated brine (100 mL), dried over anhydrous Na₂SO₄, filtered and concentrated to give 28.1 (110 mg, crude), which was used directly for next step.

### Synthesis of 28

To a solution of 28.1 (110 mg, 0.26 mmol) in acetone (10 mL) was added K₂CO₃ (107 mg, 0.78 mmol) and 1H-pyrazole-4-carbonitrile (72.1 mg, 0.78 mmol). The reaction mixture was stirred at RT for 16 h. The mixture was poured into water (50 mL) then extracted with EtOAc (3×20 mL). The combined organic layer was washed with water (100 mL), saturated brine (100 mL), dried over anhydrous Na₂SO₄, filtered and concentrated. The residue was purified by pre-HPLC (Column: Boston Prime C18 150*30 mm*5 μm, Condition: water (0.05% ammonia hydroxide v/v)-ACN, Begin B: 70, End B: 100, Gradient Time (min): 9, 100% B Hold Time (min): 2) to give 28 (50.0 mg, crude), which was further purified by pre-HPLC (Column: Boston Prime C18 150*30 mm 5 μm, Condition: water (0.05% ammonia hydroxide v/v)-ACN, Begin: 70, End B: 100, Gradient Time (min): 9, 100% B Hold Time (min): 2) to give pure 28 (10.0 mg, 20%). ¹H NMR (400 MHz, CDCl₃) δ_{H} 7.83 (s, 1H), 7.80 (s, 1H), 5.15 (d, J=18.0 Hz, 1H), 4.93 (d, J=18.0 Hz, 1H), 2.26 (d, J=2.8 Hz, 1H), 2.15-1.59 (m, 11H), 1.52-1.20 (m, 15H), 1.15-0.80 (m, 5H), 0.67 (t, J=7.6 Hz, 3H). LC-ELSD/MS: purity>99%, MS ESI calcd. for C₂₇H₃₈N₃O [M−H₂O+H]⁺420.3, found 420.3.

### Example 29 & 30: Synthesis of 1-((1S,3aS,3bR,5aR,7R,10aS,10bS,12aS)-12a-ethyl-7-hydroxy-7,10a-dimethyloctadecahydrocyclohepta[a]cyclopenta[f]naphthalen-1-yl)ethanone (29) & Synthesis of 1-(2-((1S,3aS,3bR,5aR,7R,10aS,10bS,12aS)-12a-ethyl-7-hydroxy-7,10a-dimethyloctadecahydrocyclohepta[a]cyclopenta[f]naphthalen-1-yl)-2-oxoethyl)-1H-pyrazole-4-carbonitrile (30)

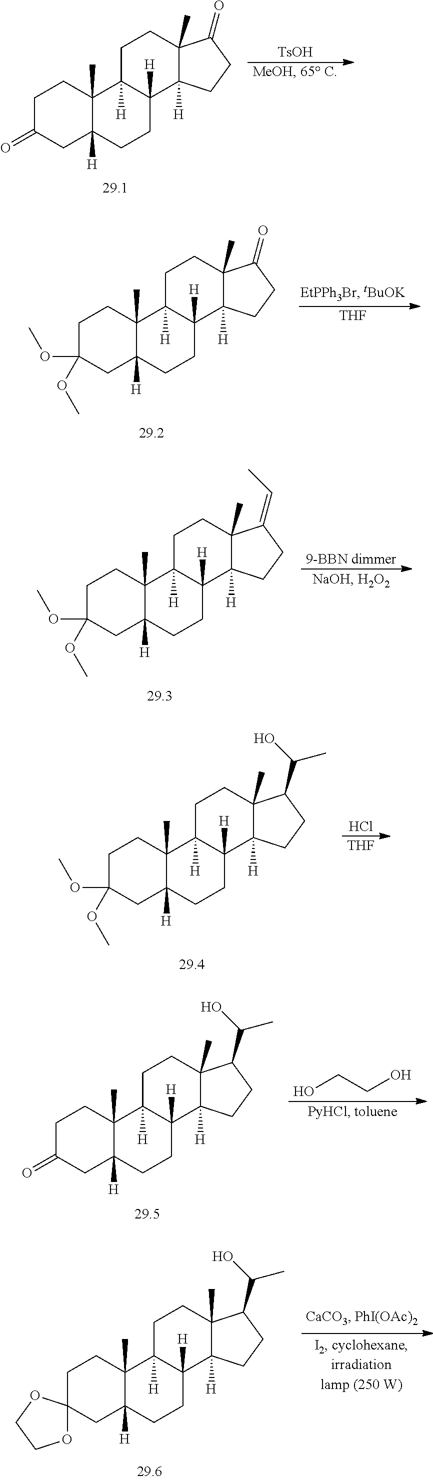

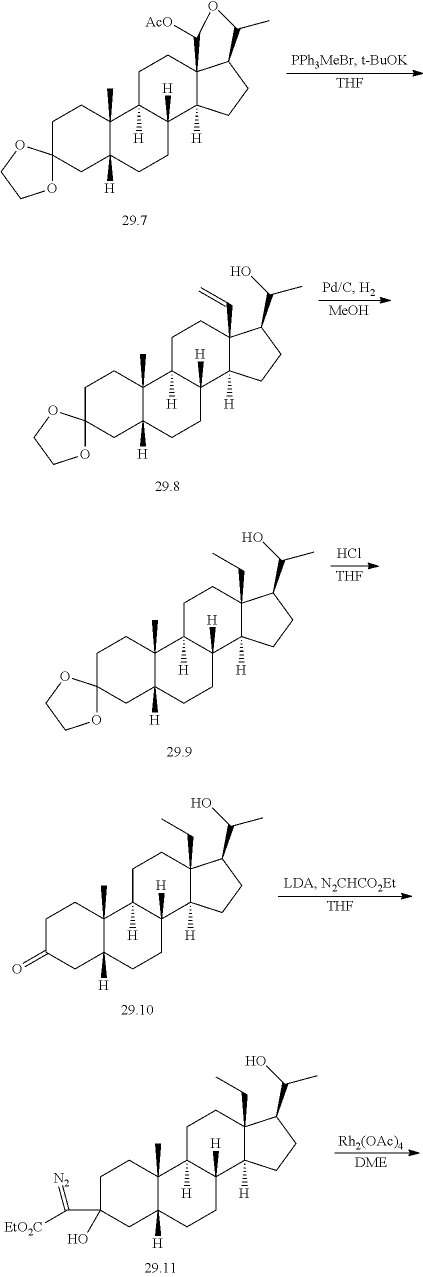

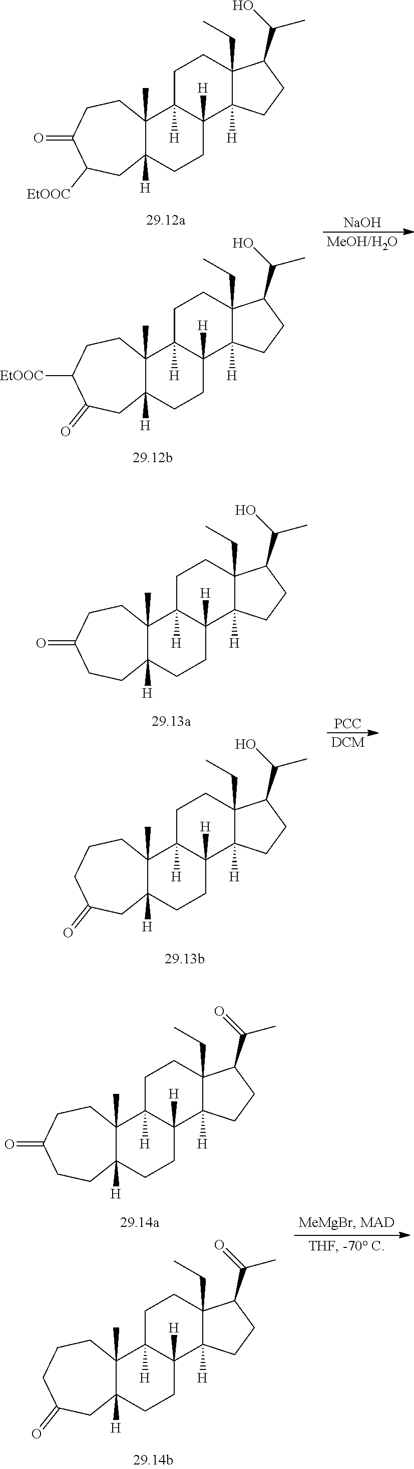

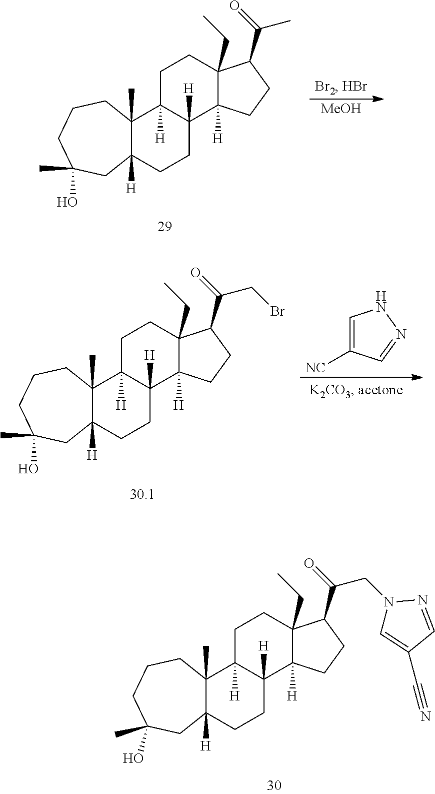

### Synthesis of 29.2

To a solution of 29.1 (10.0 g, 34.6 mmol) in anhydrous methanol (200 mL) was added TsOH (595 mg, 3.46 mmol) and the mixture was stirred at 65° C. for 18 h. Et₃N (7.40 g, 103 mmol) was added and the reaction mixture was concentrated under reduced pressure to afford 29.2 (10.0 g, crude). ¹H NMR (400 MHz, CDCl₃) δ_{H} 3.24-3.08 (m, 5H), 2.54-1.96 (m, 3H), 1.94-1.66 (m, 5H), 1.66-1.45 (m, 6H), 1.40-1.08 (m, 9H), 1.05-0.90 (m, 3H), 0.88-0.74 (m, 3H).

### Synthesis of 29.3

To a suspension of Ph₃PEtBr (22.1 g, 59.6 mmol) in anhydrous THF (400 mL) was added t-BuOK (6.67 g, 59.6 mmol) and the reaction mixture was stirred at 50° C. for 30 min. A solution of 29.2 (10.0 g, 29.8 mmol) in anhydrous THF (100 mL) was then added dropwise. After stirring at 50° C. for 16 h, the mixture was poured into saturated NH₄Cl (500 mL) and stirred for 10 min. After cooling, the suspension was extracted with EtOAc (2×200 mL). The combined organic phase was washed with saturated brine (2×500 mL), filtered and concentrated. The residue was purified by flash column (0-30% of EtOAc in PE) to give the product 29.3 (10.0 g, 97%). ¹H NMR (400 MHz, CDCl₃) S_{H} 5.18-5.02 (m, 1H), 3.49 (s, 1H), 3.23-3.12 (m, 4H), 2.46-1.99 (m, 4H), 1.92-1.70 (m, 3H), 1.68-1.36 (m, 12H), 1.36-1.04 (m, 7H), 0.95 (s, 3H), 0.91-0.82 (m, 3H).

### Synthesis of 29.4

To a solution of 29.3 (10.0 g, 28.8 mmol) in THE (200 mL) was added 9-BBN (14.0 g, 57.6 mmol) and the mixture was stirred at 45° C. for 16 h. The mixture was cooled 0° C. and ethanol (13.2 g, 288 mmol), followed by NaOH aqueous (57.6 mL, 5.0 M, 288 mmol). H₂O₂(28.8 mL, 288 mmol) was added dropwise, then the reaction was stirred at 78° C. for 1 hour. The mixture was cooled then poured into Na₂SO₃ (200 mL). After stirring for 10 min, the suspension was extracted with EtOAc (2×100 mL), The combined organic layer was washed with saturated brine (4×300 mL), drive over anhydrous Na₂SO₄, filtered and concentrated the crude product 29.4 (10.0 g, crude). ¹H NMR (400 MHz, CDCl₃) δ_{H} 3.19 (s, 3H), 3.12 (s, 3H), 2.43-2.35 (m, 1H), 1.97-1.69 (m, 12H), 1.68-1.57 (m, 7H), 1.26-1.12 (m, 9H), 0.97-0.89 (m, 3H), 0.63 (s, 3H).

### Synthesis of 29.5

To a solution 29.4 (10.0 g, 27.4 mmol) in THE (100 ml) and was added HCl (27.4 ml, 2M, 54.8 mmol) and the mixture was stirred at RT for 2 h. The pH of the mixture was adjusted to 7 with NaHCO₃. The suspension was extracted with EtOAc (2×150 mL) and the combined organic layer was washed with saturated brine (4×300 mL), then concentrated. The residue was purified by flash column (0-40% of EtOAc in PE) to give product 29.5 (7.00 g, 80.2%). ¹H NMR (400 MHz, CDCl₃) δ_{H} 3.77-3.63 (m, 1H), 2.78-2.61 (m, 1H), 2.41-2.24 (m, 1H), 2.23-2.11 (m, 1H), 2.08-1.98 (m, 2H), 1.96-1.77 (m, 4H), 1.73-1.48 (m, 6H), 1.42-1.07 (m, 12H), 1.04-0.98 (m, 3H), 0.68 (s, 3H).

### Synthesis of 29.6

To a solution of 29.5 (10.0 g, 31.3 mmol) in toluene (100 mL) was added pyridine hydrochloride (361 mg, 3.13 mmol) and ethane-1,2-diol (9.68 g, 156 mmol). The mixture was stirred at 130° C. for 16 h with a Dean-Stark trap to remove water. After cooling, saturated NaHCO₃ (200 mL) was added and the mixture was extracted with EtOAc (2×100 mL). The combined extracts were washed with H₂O (500 mL), saturated brine (300 mL), dried over anhydrous Na₂SO₄, filtered and concentrated. The residue was purified by flash column (0-40% of EtOAc in PE) to give the product 29.6 (7.00 g, 61.9%). ¹H NMR (400 MHz, CDCl₃) δ_{H} 3.94 (s, 4H), 3.77-3.63 (m, 1H), 2.05-1.81 (m, 4H), 1.76-1.59 (m, 4H), 1.55-1.31 (m, 9H), 1.28-1.09 (m, 10H), 0.95 (s, 3H), 0.65 (s, 3H).

### Synthesis of 29.7

To a solution of 29.6 (2.00 g, 5.51 mmol) in cyclohexane (200 mL) were added CaCO₃ (1.65 g, 16.5 mmol), PhI(OAc)₂ (5.31 g, 16.5 mmol), 12 (2.79 g, 11 mmol). The mixture was heated to reflux by irradiation with an infrared lamp (250 W) for 30 min. The reaction was quenched with saturated Na₂S₂O₃ (200 mL) and the suspension was extracted with EtOAc (2×100 mL). The combined organic layer was washed with brine (100 mL), dried over anhydrous Na₂SO₄, filtered and concentrated to give 29.7 (3.00 g, crude), which was used directly for the next step.

### Synthesis of 29.8

To a solution of MePh₃PBr (7.64 g, 21.4 mmol) in THF (40 mL) was added t-BuOK (2.40 g, 21.4 mmol) and the mixture was stirred at 50° C. for 1h. A solution of 29.7 (3.00 g, 7.16 mmol) in THE (10 mL) was then added and the reaction mixture was stirred at 50° C. for another 16 h. After cooling, the mixture was poured into saturated NH₄Cl (100 mL), then extracted with EtOAc (3×100 mL). The combined organic layer was washed with saturated brine (100 mL), dried over anhydrous Na₂SO₄, filtered and concentrated. The residue was purified by flash column (5-15% of EtOAc in PE) to give 29.8 (4.00 g, crude). ¹H NMR (400 MHz, CDCl₃) δ_{H} 5.76 (dd, J=18.00, 11.20 Hz, 1H), 5.29 (dd, J=10.80, 1.20 Hz, 1H), 5.14 (dd, J=18.40, 1.60 Hz, 1H), 3.93 (s, 4H), 3.85-3.77 (m, 1H), 2.32-2.40 (m, 1H), 1.73-1.87 (m, 5H), 1.62-1.70 (m, 2H), 1.54-1.58 (m, 1H), 1.44-1.54 (m, 4H), 1.31-1.39 (m, 4H), 1.24-1.31 (m, 5H), 1.16-1.22 (m, 2H), 1.11 (d, J=5.60 Hz, 3H), 0.86 (s, 3H).

### Synthesis of 29.9

To a solution of 29.8 (7.00 g, 18.6 mmol) in MeOH (50 mL) was added Pd—C (dry, 10%, 2.00 g). The suspension was degassed under vacuum, purged with H₂ for three times then hydrogenated under 50 psi of hydrogen at 50° C. for 16 h. After cooling, the reaction mixture was filtered through a pad of Celite and washed with THE (3×300 mL). The filtrate was concentrated to give the product 29.9 (7.00 g, crude). ¹H NMR (400 MHz, CDCl₃) S_{H} 3.93 (s, 4H), 3.87-3.78 (m, 1H), 2.16-2.09 (m, 1H), 1.98-1.78 (m, 3H), 1.75-1.69 (m, 1H), 1.64-1.55 (m, 5H), 1.47-1.44 (m, 1H), 1.40-1.29 (m, 6H), 1.26 (d, J=6.00 Hz, 3H), 1.25-1.15 (m, 4H), 1.15-1.00 (m, 5H), 0.93 (s, 3H), 0.84 (t, J=7.60 Hz, 3H).

### Synthesis of 29.10

To a solution of 29.9 (2.70 g, 7.16 mmol) in THE (30 mL) was added 2M HCl (7.15 mL, 14.3 mmol). The reaction mixture was stirred at RT for 16 h to give a yellow solution. The reaction was diluted with H₂O (50 mL), and the pH was adjusted to 9 with solid NaHCO₃. The mixture was extracted with EtOAc (3×50 mL). The combined organic layers were dried over Na₂SO₄, filtered and concentrated to give the product 29.10 (2.20 g, crude). ¹H NMR (400 MHz, CDCl₃) δ_{H} 3.89-3.81 (m, 1H), 2.77-2.63 (m, 1H), 2.39-2.28 (m, 1H), 2.23-2.12 (m, 2H), 2.02-1.99 (m, 1H), 1.97-1.75 (m, 4H), 1.69-1.62 (m, 2H), 1.57-1.44 (m, 5H), 1.41-1.29 (m, 4H), 1.27 (d, J=6.00 Hz, 3H), 1.23-1.07 (m, 6H), 1.01 (s, 3H), 0.87 (t, J=7.60 Hz, 3H).

### Synthesis of 29.11

At −70° C., an LDA solution (13.2 mL, 2M, 26.4 mmol) was added to a stirred solution of 29.10 (2.20 g, 6.61 mmol) and ethyl diazoacetate (3.01 g, 26.4 mmol) in THF (200 mL). After stirring at −70° C. for 2 h, HOAc (1.58 g, 26.4 mmol) in THF (10 mL) was added. The mixture was warmed to RT and stirred for 16 h. Water (500 mL) and PE (300 mL) was added and the suspension was extracted with EtOAc (2×500 mL). The combined organic layers were washed with saturated brine (600 mL), dried over anhydrous Na₂SO₄, filtered and concentrated to give the 29.11 (4.00 g, crude). ¹H NMR (400 MHz, CDCl₃) δ_{H} 4.23 (q, J=7.20 Hz, 2H), 3.88-3.77 (m, 1H), 2.15-2.10 (m, 1H), 2.02-1.87 (m, 3H), 1.80-1.70 (m, 2H), 1.69-1.52 (m, 6H), 1.51-1.43 (m, 3H), 1.41-1.31 (m, 5H), 1.31-1.30 (m, 1H), 1.26 (s, 3H), 1.22-1.18 (m, 3H), 1.17-0.99 (m, 6H), 0.94 (d, J=12.40 Hz, 3H), 0.87-0.81 (m, 3H).

### Synthesis of 29.12a & 29.12b

To a solution of 29.11 (4.00 g, 8.95 mmol) in DME (50 mL) was added Rh₂(OAc)₄ (79.1 mg, 0.179 mmol) and the reaction mixture was stirred at RT for 16 h. The reaction mixture was concentrated to give a mixture 29.12a & 29.12b (5.00 g, crude). 1H NMR (400 MHz, CDCl₃) S_{H} 4.14-4.21 (m, 2H), 3.87-3.79 (m, 1H), 2.27-2.72 (m, 2H), 2.08-2.21 (m, 2H), 1.79-1.94 (m, 4H), 1.65-1.78 (m, 3H), 1.52-1.63 (m, 4H), 1.44-1.49 (m, 2H), 1.44-1.51 (m, 3H), 1.34-1.39 (m, 4H), 1.27 (d, J=6.40 Hz, 6H), 1.03-1.16 (m, 5H), 0.97-0.93 (m, 3H), 0.85 (t, J=7.20 Hz, 3H).

### Synthesis of 29.13a & 29.13b

To a mixture of 29.12a & 29.12b (5.00 g, 11.9 mmol) in MeOH (60 mL) was added H₂O (20 mL) and NaOH (4.75 g, 119 mmol). The reaction mixture was stirred at 60° C. for 16 h then concentrated. Then H₂O (100 mL) was added and the mixture was extracted with EtOAc (2×100 mL). The combined organic phase was washed with saturated brine (100 mL), dried over anhydrous Na₂SO₄, filtered and concentrated to give the mixture product 29.13a & 29.13b (1.50 g, 36.4%). ¹H NMR (400 MHz, CDCl₃) δ_{H} 3.89-3.76 (m, 1H), 3.07 (t, J=12.80 Hz, 1H), 2.55-2.23 (m, 3H), 2.22-2.06 (m, 1H), 1.99-1.52 (m, 11H), 1.50-1.35 (m, 6H), 1.16-1.06 (m, 3H), 1.01 (s, 1H), 0.98-0.95 (m, 2H), 0.85 (t, J=7.60 Hz, 3H).

### Synthesis of 29.14a & 29.14b

To a solution of 29.13a & 29.13b (500 mg, 1.44 mmol) in DCM (10 mL) was added silica gel (500 mg) and PCC (462 mg, 2.15 mmol) and the mixture was stirred at 25° C. for 1 h. The suspension was filtered, and the filter cake was washed with DCM (2×20 mL). The combined filtrate was concentrated and the residue was purified by flash column (0-60% of EtOAc in PE) to give a crude mixture of 29.14a & 29.14b (900 mg), which was separated by SFC (Column: DAICEL CHIRALPAK AD (250 mm*30 mm*10 μm)), Condition: A: CO₂ B: 0.1% NH₃H₂O EtOH; Begin B: 30%, End B: 30%, Flow Rate (ml/min):70) to afford 29.14a (200 mg, 22.2%) and 29.14b (500 mg, 55.6%).

29.14a: ¹H NMR (400 MHz, CDCl₃) δ_{H} 2.72-2.35 (m, 4H), 2.30-2.15 (m, 3H), 2.14-2.12 (m, 3H), 2.10-1.98 (m, 1H), 1.91-1.77 (m, 1H), 1.73-1.63 (m, 1H), 1.61-1.53 (m, 2H), 1.49-1.31 (m, 6H), 1.30-0.98 (m, 9H), 0.95 (s, 3H), 0.95-0.79 (m, 1H), 0.60-0.54 (m, 3H).

29.14b: ¹H NMR (400 MHz, CDCl₃) δ_{H} 3.12-2.99 (m, 1H), 2.55-2.43 (m, 2H), 2.42-2.31 (m, 2H), 2.20 (s, 3H), 2.01-1.85 (m, 2H), 1.84-1.71 (m, 2H), 1.70-1.60 (m, 3H), 1.58-1.42 (m, 4H), 1.42-0.99 (m, 10H), 0.97 (s, 3H), 0.94-0.84 (m, 1H), 0.64 (t, J=7.60 Hz, 3H).

### Synthesis of 29

A freshly prepared MAD (4.35 mmol in 10 mL toluene) solution was cooled to −70° C. then added dropwise to a cooled (−70° C.) and stirred solution of 29.14b (400 mg, 1.16 mmol) in DCM (5 mL). After stirring at −70° C. for 1 h, MeMgBr (1.93 mL, 5.8 mmol, 3M in ethyl ether) was added dropwise and the resulting solution was stirred at −70° C. for another 4 h. The reaction mixture was poured into saturated aqueous citric acid (50 mL) and extracted with EtOAc (2×50 mL). The combined organic layer was dried over Na₂SO₄, filtered and concentrated. The residue was purified by a silica gel column (PE/EtOAc=0-20%) to give 29 (250 mg, 59.7%). ¹H NMR (400 MHz, CDCl₃) δ_{H} 2.52-2.41 (m, 1H), 2.37-2.28 (m, 1H), 2.20 (s, 3H), 2.11-1.99 (m, 1H), 1.96-1.57 (m, 6H), 1.52-1.42 (m, 3H), 1.41-1.27 (m, 6H), 1.26 (s, 3H), 1.25-1.16 (m, 5H), 1.16-0.95 (m, 4H), 0.88 (s, 3H), 0.63 (t, J=7.60 Hz, 3H). LC-ELSD/MS: purity >99%; MS ESI calcd. for C₂₄H₃₉O [M−H₂O+H]⁻ 343.3, found 343.3.

### Synthesis of 30.1

At 0° C., to a solution of 29 (100 mg, 0.2773 mmol) and HBr (11.0 mg, 0.055 mmol, 40%) in MeOH (10 mL) was added Br₂ (48.8 mg, 0.305 mmol) and the mixture was stirred at 25° C. for 2 h. The mixture was poured into saturated NaHCO₃ (50 mL) then extracted with EtOAc (3×20 mL). The combined organic layer was washed with saturated brine (50 mL), dried over anhydrous Na₂SO₄, filtered and concentrated to give 30.1 (130 mg, crude). ¹H NMR (400 MHz, CDCl₃) δ_{H} 4.01 (s, 2H), 2.80-2.67 (m, 1H), 2.38-2.13 (m, 3H), 1.99-1.88 (m, 1H), 1.87-1.72 (m, 4H), 1.71-1.59 (m, 4H), 1.53-1.39 (m, 6H), 1.38-1.29 (m, 6H), 1.26 (s, 3H), 1.08-0.94 (m, 4H), 0.88 (s, 3H), 0.59 (t, J=7.60 Hz, 3H).

### Synthesis of 30

To a solution of 30.1 (130 mg, 0.296 mmol) in acetone (5 ml) was added K₂CO₃ (81.6 mg, 0.5916 mmol) and 1H-pyrazole-4-carbonitrile (30.2 mg, 0.325 mmol) and the mixture was stirred at RT for 2 h. The mixture was poured into saturated water (50 mL) and stirred for 10 min. The suspension was extracted with EtOAc (2×50 mL) and the combined organic phase was washed with saturated brine, filtered and concentrated. The residue was purified by flash column (0˜30% of EtOAc in PE) to give 30 (30.0 mg, 22.5%). ¹H NMR (400 MHz, CDCl₃) δ_{H} 7.86 (s, 1H), 7.81 (s, 1H), 5.16 (d, J=18.40 Hz, 1H), 4.92 (d, J=18.00 Hz, 1H), 2.52-2.45 (m, 1H), 2.37-2.26 (m, 2H), 2.08-2.01 (m, 1H), 1.88-1.64 (m, 3H), 1.53-1.39 (m, 6H), 1.39-1.29 (m, 7H), 1.26 (s, 3H) 1.24-1.10 (m, 6H), 1.09-1.00 (m, 2H), 0.89 (s, 3H), 0.61 (t, J=7.60 Hz, 3H). LC-ELSD/MS: purity >99%; MS ESI calcd. for C₂₈H₄₀N₃O₁ [M−H₂O+H]434.3, found 434.3.

### Example 31 & 32: Synthesis of 1-(2-((1S,3aS,3bR,5aR,8S,10aS,10bR,12aS)-12a-ethyl-8-hydroxy-8-(methoxymethyl)octadecahydrocyclohepta[a]cyclopenta[f]naphthalen-1-yl)-2-oxoethyl)-1H-pyrazole-4-carbonitrile (31) & Synthesis of 1-((1S,3aS,3bR,5aR,8S,10aS,10bR,12aS)-12a-ethyl-8-hydroxy-8-(methoxymethyl)octadecahydrocyclohepta[a]cyclopenta[f]naphthalen-1-yl)ethanone (32)

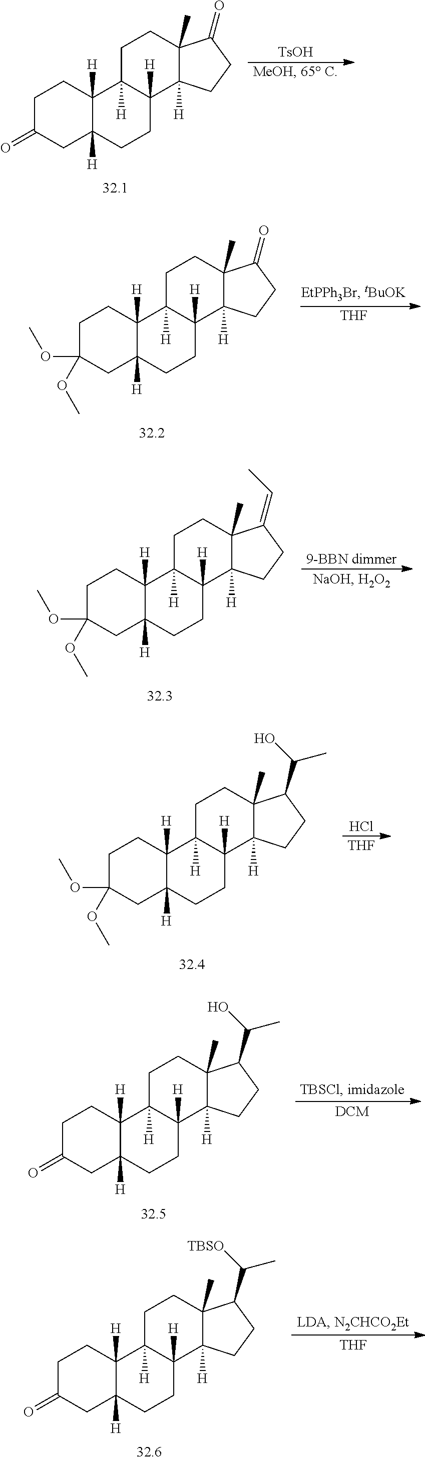

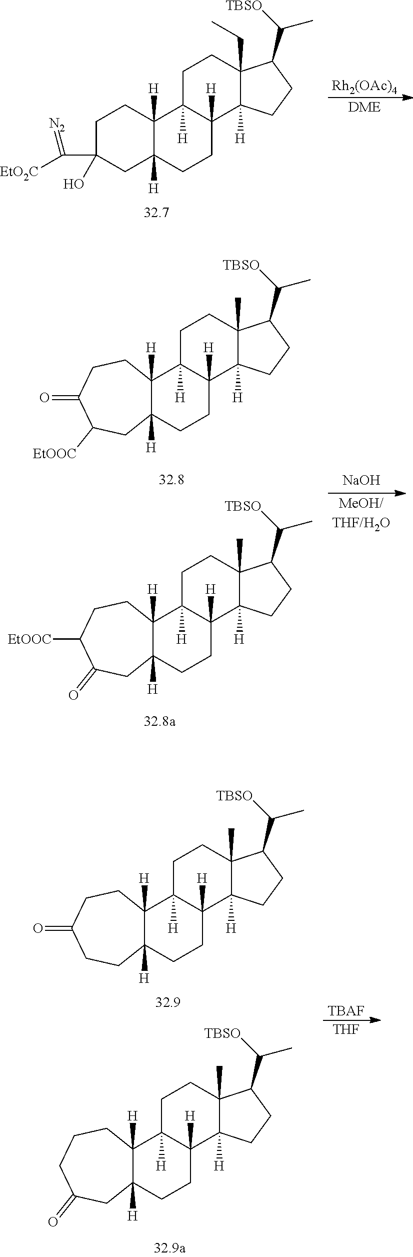

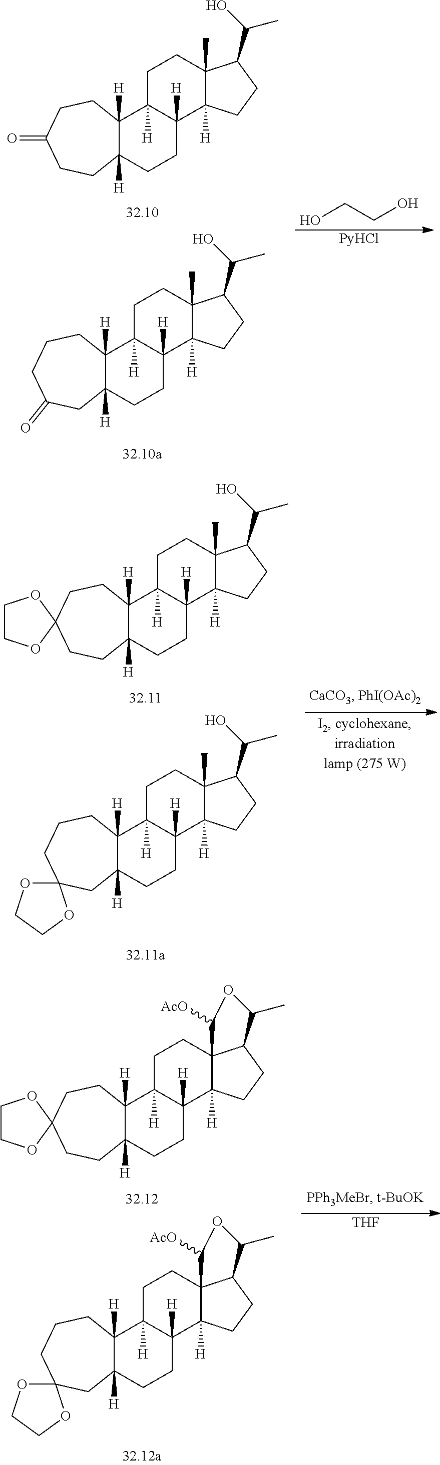

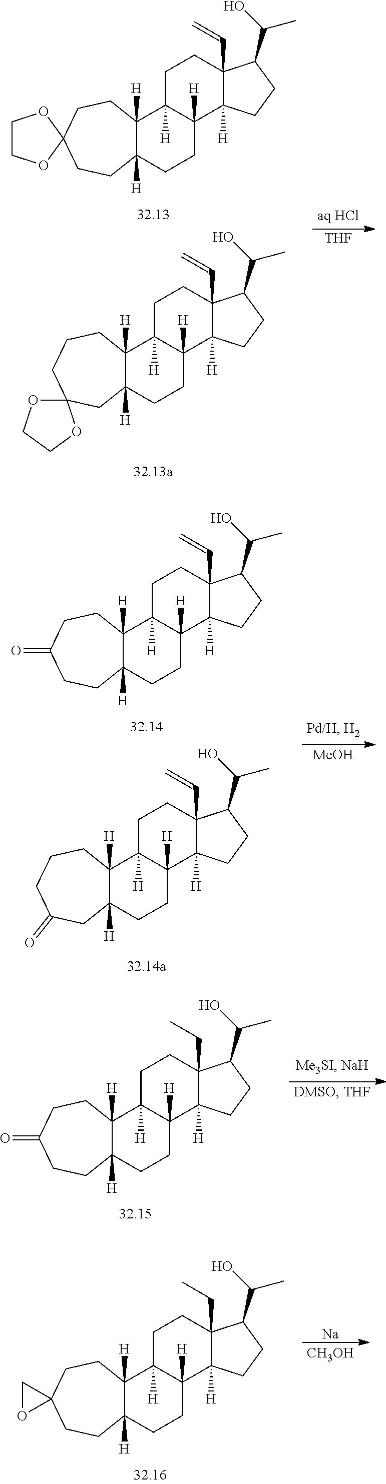

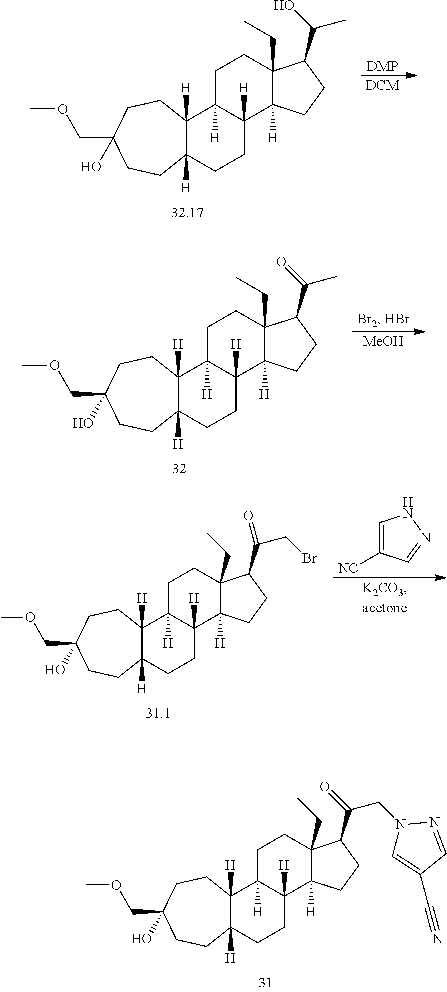

### Synthesis of 32.2

To a solution of 32.1 (300 g, 1093 mmol, reported in patent ‘WO2014/169833, 2014, A1’) in MeOH (2.0 L) was added TsOH (18.7 g, 109 mmol) and the mixture was stirred at 65° C. for 1 h. The reaction mixture was cooled, and the precipitate was collected by filtration and washed with methanol (2×300 mL) to give 32.2 (230 g, crude). ¹H NMR (400 MHz, CDCl₃) δ_{H} 3.19 (s, 3H), 3.14 (s, 3H), 2.60-2.39 (m, 2H), 2.25-2.00 (m, 2H), 1.97-1.90 (m, 2H), 1.86-1.75 (m, 6H), 1.70-1.60 (m, 5H), 1.56-1.49 (m, 4H), 1.47-1.35 (m, 10H), 1.30-1.22 (m, 5H), 1.15-1.00 (m, 2H), 0.86 (s, 3H).

### Synthesis of 32.3

To a suspension of EtPPh₃Br (798 g, 2.15 mol) in THE (1.5 L) was added t-BuOK (241 g, 2.15 mol) and the mixture was stirred at 50° C. for 30 min. After cooling, the mixture was added 32.2 (230 g, 717 mmol) in THE (500 mL) and the mixture was stirred at 50° C. for another 16 h. The reaction was cooled, quenched with sat NH₄Cl (500 mL) and extracted with EtOAc (2×500 mL). The combined organic phase was washed with brine (2×500 mL), dried over anhydrous Na₂SO₄, filtered and concentrated to give crude product, which was triturated with 1:1 methanol (1 L)/water (1 L) to give the product 32.3 (290 g, crude). 1H NMR (400 MHz, CDCl₃) δ_{H} 3.19 (s, 3H), 3.14 (s, 3H), 2.40-2.10 (m, 4H), 1.95-1.35 (m, 13H), 1.33-1.05 (m, 10H), 0.87 (s, 3H).

### Synthesis of 32.4

To a solution of 32.3 (275 g, 826 mmol) in THE (2 L) was added 9-BBN dimer (402 g, 1.65 mol) and the mixture was stirred at 50° C. for 2 h. The reaction mixture was cooled to 0° C. and ethanol (379 g, 8.26 mol) was carefully added followed by NaOH (1.65 L, 5 M, 8.26 mol). After the addition was complete, H₂O₂(825 mL, 8.26 mol, 30%) was added dropwise while the internal temperature was maintained below 15° C. The resulting solution was slowly heated to 75° C. and stirred for 1 h. After cooling, saturated aqueous Na₂S₂O₃ (260 mL) was added and the mixture was stirred at 0° C. for another 1 h. Water (2 L) was added and the mixture was stirred at 0° C. for 30 min, then filtered. The filter cake was washed with water (3×700 mL), dried under vacuum to give 32.4 (285 g, crude).

### Synthesis of 32.5

To a solution of 32.4 (285 g, 813 mmol) in THE (3 L) was added aqueous HCl (1.62 L, 1.62 mol, 1 M) and the mixture was stirred for 1 h. Water (700 mL) was added and the mixture was extracted with DCM (2×500 mL). The combined organic phase was washed with brine (2×500 mL), dried over anhydrous Na₂SO₄, filtered, concentrated to afford 32.5 (280 g, crude). ¹H NMR (400 MHz, CDCl₃) δ 3.75-3.65 (m, 1H), 2.65-2.55 (m, 1H), 2.30-2.10 (m, 1H), 2.00-1.80 (m, 5H), 1.75-1.42 (m, 10H), 1.40-1.28 (m, 4H), 1.29-1.15 (m, 7H), 0.66 (s, 3H).

### Synthesis of 32.6

To a solution of 32.5 (10.0 g, 32.8 mmol) in DCM (40 mL) were added imidazole (4.46 g, 65.6 mmol) and TBSCl (9.88 g, 65.6 mmol) and the reaction mixture was stirred at RT for 1 h. The reaction mixture was filtered, and the filtrate was washed with saturated NH₄Cl (2×60 mL), dried over anhydrous Na₂SO₄, filtered and concentrated. The residue was purified by flash column (0-15% of EtOAc in PE) to give 32.6 (8.20 g, 62%). ¹H NMR (400 MHz, CDCl₃) δ_{H} 3.73-3.62 (m, 1H), 2.60 (t, 0.1=14.0 Hz, 1H), 2.29-2.06 (m, 5H), 1.96-1.85 (m, 2H), 1.77-1.66 (m, 2H), 1.61-1.44 (m, 6H), 1.36-1.09 (m, 11H), 0.87 (s, 9H), 0.67 (s, 3H), 0.06-0.03 (m, 6H).

### Synthesis of 32.7

An LDA solution (23.8 mmol) was cooled −70° C., then added to a stirred solution of 32.6 (2.00 g, 4.8 mmol) and ethyl 2-diazoacetate (2.71 g, 23.8 mmol) in THF (20 mL) at −78° C. After stirring at −70° C. for 1 h, HOAc (1.42 g, 23.8 mmol) in THF (20 mL) was added and the mixture was warmed to 20° C. Water (60 mL) was added and the mixture was extracted with EtOAc (3×50 mL). The combined organic layers were washed with saturated brine (50 mL), dried over anhydrous Na₂SO₄, and concentrated under reduced pressure to give 32.7 (2.54 g, crude).

### Synthesis of 32.8 & 32.8a

To a solution of 32.7 (2.54 g, crude) in DME (10 mL) was added Rh₂(OAc)₄ (31.5 mg, 0.071 mmol). The reaction mixture was stirred at RT for 12 h then extracted with ethyl acetate (3×20 mL). The combined organic phase was washed with water (30 mL), saturated brine (30 mL), dried over anhydrous Na₂SO₄, filtered and concentrated. The residue was purified by flash column (0-15% of EtOAc in PE) to give the mixture of 32.8 & 32.8a (2.80 g, impure).

### Synthesis of 32.9 & 32.9a

To a mixture of 32.8 & 32.8a (2.80 g, 5.5 mmol) in MeOH/THF/H₂O (20 mL/20 mL/5 mL) was added NaOH (2.21 g, 55.4 mmol). The reaction mixture was stirred at 70° C. for 12 h, then extracted with ethyl acetate (4×50 mL). The combined organic phase was washed with water (50 mL), saturated brine (60 mL), dried over anhydrous Na₂SO₄, filtered and concentrated. The residue was purified by flash column (0-15% of EtOAc in PE) to give the mixture of 32.9 & 32.9a (1.60 g, 67.3%).

### Synthesis of 32.10 & 32.10a

To a solution of 32.9 &3 2.9a (1.60 g, 3.7 mmol) in THE (50 mL) was added TBAF-3H₂O (5.74 g, 18.4 mmol). After stirring at 55° C. for 12 h, the mixture was poured into water (50 mL) and extracted with EtOAc (2×30 mL). The organic layer was washed with saturated brine (2×50 mL), dried over anhydrous Na₂SO₄, filtered and concentrated. The residue was purified by flash column (0-15% of EtOAc in PE) to give the mixture of 32.10 & 32.10a (900 mg, 76%).

### Synthesis of 32.11 & 32.11a

A mixture of 32.10 (5.00 g, 15.6 mmol, include 32.10a), ethane-1,2-diol (4.80 g, 78.1 mmol) and pyridine hydrochloride (359 mg, 3.1 mmol) in toluene (100 mL) was stirred at 135° C. for 72 h. After cooling, the mixture was poured into saturated NaHCO₃ (100 mL) and extracted with EtOAc (3×50 mL). The combined organic layer was washed with saturated brine (200 mL), dried over anhydrous Na₂SO₄, filtered and concentrated to give 32.11 (7.00 g, crude, include 32.11a). ¹H NMR (400 MHz, CDCl₃) δ_{H} 4.00-3.78 (m, 4H), 2.28-2.15 (m, 1H), 1.99-1.60 (m, 12H), 1.52-1.28 (m, 9H), 1.25-0.85 (m, 9H), 0.67 (s, 3H).

### Synthesis of 32.12 & 32.12a

To a solution of 32.11 (2.50 g, 6.9 mmol, include 32.11a), CaCO₃ (2.06 g, 20.6 mmol) and PhI(OAc)₂ (6.60 g, 20.6 mmol) in cyclohexane (250 mL) was added I₂ (3.50 g, 13.7 mmol). The mixture was heated to 80° C. by irradiation with infrared lamp (275 W) for 30 min. The mixture was cooled to RT then extracted with EtOAc (3×100 mL). The combined organic layer was washed with saturated brine (500 mL), dried over anhydrous Na₂SO₄, filtered and concentrated. The residue was purified by flash column (0-5% of EtOAc in PE) to give crude 32.12 (3.20 g, include 32.12a). ¹H NMR (400 MHz, CDCl₃)⁶H 5.95 (s, 1H), 4.30-4.20 (m, 1H), 4.00-3.78 (m, 4H), 2.40-2.15 (m, 2H), 1.99-1.59 (m, 13H), 1.52-1.28 (m, 6H), 1.25-1.15 (m, 4H), 1.10-0.75 (m, 7H).

### Synthesis of 32.13 & 32.13a

A solution of PPh₃MeBr (10.8 g, 30.5 mmol) and t-BuOK (3.40 g, 30.5 mmol) in THF (30 mL) was stirred for 1 h at 50° C. Then a solution of 32.12 (3.20 g, 7.6 mmol, include 32.12a) in THF (20 mL) was added and stirred at 50° C. for another 16 h. The mixture was poured into saturated NH₄Cl (100 mL) and extracted with EtOAc (3×50 mL). The combined organic layer was washed with saturated brine (200 mL), dried over anhydrous Na₂SO₄, filtered and concentrated. The residue was purified by flash column (0-10% of EtOAc in PE) to give 32.13 (2.20 g, 77%, include 32.13a). ¹H NMR (400 MHz, CDCl₃) OH 5.77 (dd, J=18.0 Hz, 11.2 Hz, 1H), 5.28 (dd, J=11.2 Hz, 1.2 Hz, 1H), 5.15 (dd, J=18.0 Hz, 1.6 Hz, 1H), 4.00-3.75 (m, 5H), 2.40-2.15 (m, 1H), 1.95-1.59 (m, 8H), 1.52-1.28 (m, 10H), 1.25-0.80 (m, 11H).

### Synthesis of 32.14 & 32.14a

To a solution of 32.13 (2.20 g, 5.7 mmol, include 32.13a) in THE (10 mL) was added HCl (2.4 mL, 28.7 mmol). The mixture was stirred at RT for 2 h. The mixture was poured into saturated NaHCO₃ (100 mL) and extracted with EtOAc (3×50 mL). The combined organic layer was washed with saturated NaHCO₃ (100 mL), saturated brine (200 mL), dried over anhydrous Na₂SO₄, filtered and concentrated to give 32.14 (1.90 g, crude, include 32.14a), which was further purified by SFC (Column: DAICEL CHIRALCEL OD (250 mm*50 mm*10 μm), Condition: 0.1% NH₃₀H, EtOH, Begin B: 30%, End B: 30%, Flow rate (ml/min): 200) to give 32.14 (770 mg, 41%) and 32.14a (500 mg, 26%).

32.14: ¹H NMR (400 MHz, CDCl₃)^{6H} 5.78 (dd, J=18.0 Hz, 11.2 Hz, 1H), 5.30 (dd, J=11.2 Hz, 1.2 Hz, 1H), 5.16 (dd, J=18.0 Hz, 1.6 Hz, 1H), 3.91-3.75 (m, 1H), 2.61-2.25 (m, 5H), 1.99-1.59 (m, 9H), 1.52-1.30 (m, 6H), 1.28-0.95 (m, 10H).

32.14a: ¹H NMR (400 MHz, CDCl₃) δ_{H} 5.78 (dd, J=18.0 Hz, 11.2 Hz, 1H), 5.30 (dd, J=11.2 Hz, 1.2 Hz, 1H), 5.16 (dd, J=18.0 Hz, 1.6 Hz, 1H), 3.91-3.75 (m, 1H), 3.03 (t, J=12.4 Hz, 1H), 2.50-2.31 (m, 3H), 2.10-1.59 (m, 10H), 1.52-1.20 (m, 9H), 1.18-0.90 (m, 7H).

### Synthesis of 32.15

A suspension of 32.14 (700 mg, 2.1 mmol) and dry Pd/C (10%, 100 mg) in MeOH (20 mL) was stirred at RT for 16 h under 15 psi of H₂. The mixture was filtered, and the filter cake was washed with EtOAc (3×20 mL). The filtrate was concentrated to give 32.15 (600 mg, 86%). ¹H NMR (400 MHz, CDCl₃) δ_{H} 4.85-4.65 (m, 1H), 3.90-3.75 (m, 1H), 2.60-2.40 (m, 3H), 2.35-2.20 (m, 1H), 1.99-1.75 (m, 8H), 1.52-1.20 (m, 11H), 1.15-0.85 (m, 11H).

### Synthesis of 32.16

At 0° C., to a stirred solution of trimethylsulfonium iodide (771 mg, 3.8 mmol) in DMSO (10 mL) and THF (5 mL) was added NaH (151 mg, 3.8 mmol, 60% in mineral oil). The mixture was stirred at 0° C. for 1.0 h. A solution of 32.15 (630 mg, 1.9 mmol) in DMSO (5 mL) was added and the mixture was stirred at 25° C. for 16 h. The reaction was treated with water (100 mL) and the mixture was extracted with EtOAc (3×50 mL). The combined organic phase was washed with water (2×100 mL), saturated brine (100 mL), dried over anhydrous Na₂SO₄, filtered, and concentrated to give 32.16 (600 mg, crude). ¹H NMR (400 MHz, CDCl3)^{6H} 3.90-3.75 (m, 1H), 2.65-2.52 (m, 2H), 2.16-2.06 (m, 1H), 2.00-1.62 (m, 8H), 1.52-1.25 (m, 10H), 1.22-0.95 (m, 11H), 0.92-0.80 (m, 5H).

### Synthesis of 32.17

At 0° C., Na (832 mg, 34.6 mmol) was added in small portions to MeOH (20 mL) and the mixture was slowly heated to 70° C. and stirred for 4 h. After cooling, a solution of 32.16 (600 mg, 1.7 mmol) in MeOH (10 mL) was added and the mixture was stirred at 70° C. for 16 h. The mixture was cooled and poured into water (100 mL). The suspension was extracted with EtOAc (3×50 mL). The combined organic layer was washed with saturated brine (200 mL), dried over anhydrous Na₂SO₄, filtered and concentrated. The residue was purified by flash column (0-10% of EtOAc in PE) to give 32.17 (540 mg, 83%). ¹H NMR (400 MHz, CDCl₃) OH 3.90-3.75 (m, 1H), 3.42-3.38 (m, 3H), 3.22 (s, 1H), 3.16 (s, 1H), 2.38-2.22 (m, 2H), 1.95-1.60 (m, 7H), 1.52-1.25 (m, 14H), 1.22-0.80 (m, 13H).

### Synthesis of 32

A solution of 32.17 (540 mg, 1.4 mmol) and Dess Martin Reagent (1.80 g, 4.3 mmol) in DCM (20 mL) was stirred at RT for 1 h. The reaction was quenched with saturated NaHCO₃ aqueous solution (200 mL) then extracted with DCM (3×50 mL). The combined organic layer was washed with saturated NaHCO₃/Na₂S₂O₃ aqueous (1:1, 2×100 mL), saturated brine (200 mL), dried over anhydrous Na₂SO₄, filtered and concentrated. The residue was purified by flash column (0-15% of EtOAc in PE) to give 32.18 (420 mg, 79%), which was further purified by prep-HPLC (Column: Xbridge 150*30 mm*10 μm, Condition: water (10 mM NH₄HCO₃)-ACN, Begin B: 75, End B: 95, Gradient Time (min): 7, 100% B Hold Time (min): 0, Flow rate (ml/min): 25, Injections: 12) to give 32 (148 mg, 35%).

32: ¹H NMR (400 MHz, CDCl₃)⁶H 3.40 (s, 3H), 3.30-3.20 (m, 2H), 2.46 (t, J=8.8 Hz, 1H), 2.40-2.18 (m, 6H), 1.99-1.91 (m, 1H), 1.90-1.60 (m, 7H), 1.52-1.15 (m, 14H), 1.10-0.85 (m, 3H), 0.62 (t, J=7.6 Hz, 3H).

### Synthesis of 31.1

At 0° C., to a solution of 32 (140 mg, 0.37 mmol) in MeOH (5 mL) was added HBr (14.8 mg, 0.07 mmol, 40%) and Br₂ (59.4 mg, 0.37 mmol), and the reaction mixture was stirred at RT for 16 h. The mixture was poured into saturated NaHCO₃ (50 mL) then extracted with EtOAc (3×20 mL). The combined organic layer was washed with saturated brine (100 mL), dried over anhydrous Na₂SO₄, filtered and concentrated to give 31.1 (150 mg, crude), which was used for next step directly.

### Synthesis of 31

A solution of 31.1 (140 mg, 0.3 mmol), 1H-pyrazole-4-carbonitrile (57.2 mg, 0.6 mmol) and K₂CO₃ (84.8 mg, 0.6 mmol) in acetone (10 mL) was stirred at RT for 16 h. The mixture was poured into water (50 mL) then extracted with EtOAc (3×20 mL). The combined organic layer was washed with water (100 mL), saturated brine (100 mL), dried over anhydrous Na₂SO₄, filtered and concentrated. The residue was purified by prep-HPLC (Column: Boston Prime C18 150*30 mm*5 μm, Condition: water (0.05% ammonia hydroxide v/v)-ACN, Begin: 70, End B: 100, Gradient Time (min): 9, 100% B Hold Time (min): 2) to give 31 (41.0 mg, 29%). ¹H NMR (400 MHz, CDCl₃)⁶H 7.86 (s, 1H), 7.81 (s, 1H), 5.16 (d, J=18 Hz, 111), 4.92 (d, J=18 Hz, 1H), 3.39 (s, 3H), 3.17 (s, 2H), 2.49 (t, J=8.4 Hz, 1H), 2.40-2.20 (m, 2H), 2.11 (s, 1H), 1.95-1.59 (m, 8H), 1.52-1.35 (m, 6H), 1.33-0.85 (m, 11H), 0.60 (t, J=7.6 Hz, 3H). LC-ELSD/MS purity 99%, MS ESI calcd. for C₂₈H₄₀N₃O₂[M−H₂O+H]⁻ 450.3, found 450.3.

### Example 33: Synthesis of 1-(2-((1S,3aS,3bR,5aR,8S,10aS,10bR,12aS)-12a-ethyl-8-hydroxy-8-methyloctadecahydrocyclohepta[ajcyclopenta[f]naphthalen-1-yl)-2-oxoethyl)-1H-pyrazole-4-carbonitrile (33)

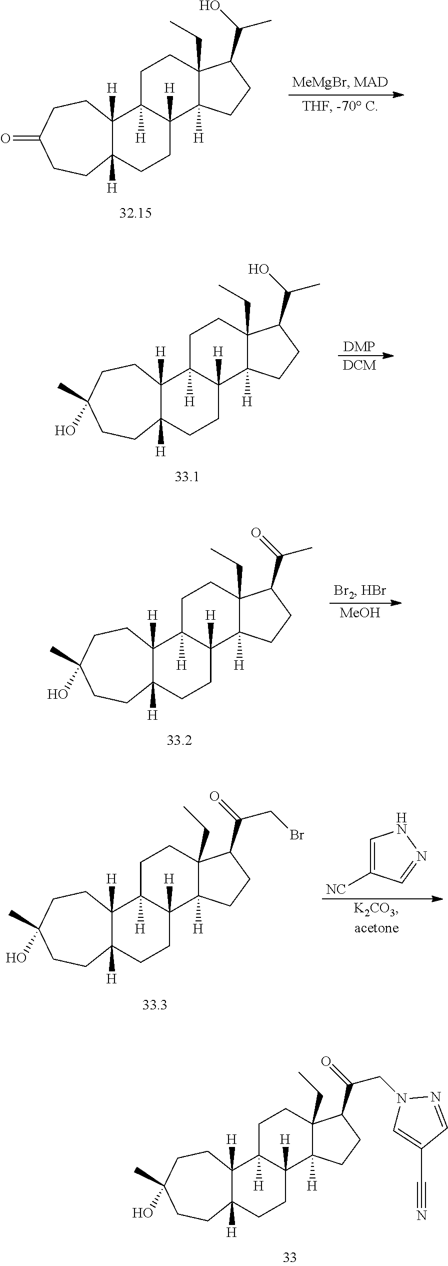

### Synthesis of 33.1

At −70° C., to a freshly prepared solution of MAD (5.4 mmol) in toluene (10 m) was added a solution of 32.15 (600 mg, 1.8 mmol) in DCM (10 mL). The reaction mixture was stirred at −70° C. for 1h then MeMgBr (3.0 mL, 9.0 mmol, 3M) was added. The reaction mixture was stirred at −70° C. for another 2 h then poured into saturated citric acid (100 mL) below 10° C. The suspension was extracted with EtOAc (3×50 mL) and the combined organic layer was washed with saturated brine (200 mL), dried over anhydrous Na₂SO₄, filtered and concentrated. The residue was purified by flash column (0-8% of EtOAc in PE) to give 33.1 (200 mg, 32.1%)

33.1: ¹H NMR (400 MHz, CDCl₃) δ_{H} 3.91-3.75 (m, 1H), 2.14-2.09 (m, 1H), 1.95-1.62 (m, 5H), 1.55-1.31 (m, 12H), 1.30-0.95 (m, 14H), 0.90-0.78 (m, 7H).

### Synthesis of 33.2

A solution of 33.1 (100 mg, impure) and Dess Martin Reagent (243 mg, 0.57 mmol) in DCM (5 mL) was stirred at RT for 16 h. The mixture was poured into saturated NaHCO₃ (200 mL) and extracted with DCM (3×20 mL). The combined organic layer was washed with saturated Na₂S₂O₃ (2×100 mL), saturated brine (200 mL), dried over anhydrous Na₂SO₄, filtered and concentrated to give 33.2 (80.0 mg, crude). ¹H NMR (400 MHz, CDCl₃) δ_{H} 2.46 (t, J=8.8 Hz, 1H), 2.40-2.18 (m, 5H), 1.90-1.59 (m, 10H), 1.52-1.25 (m, 7H), 1.24-0.85 (m, 12H), 0.63 (t, J=7.6 Hz, 3H).

### Synthesis of 33.3

At 0° C., to a solution of 33.2 (80.0 mg, 0.23 mmol) and HBr (10 mg, 0.05 mmol, 40%) in MeOH (10 mL) was added Br₂ (36.9 mg, 0.23 mmol) and the mixture was stirred at 25° C. for 2 h. The mixture was poured into saturated NaHCO₃ (50 mL) then extracted with EtOAc (3×20 mL). The combined organic layer was washed with saturated brine (100 mL), dried over Na₂SO₄, filtered and concentrated to give 33.3 (100 mg, crude). ¹H NMR (400 MHz, CDCl₃)⁶H 4.08-3.95 (m, 2H), 2.81-2.69 (m, 1H), 2.40-2.20 (m, 3H), 1.95-1.59 (m, 7H), 1.52-1.32 (m, 7H), 1.30-0.80 (m, 14H), 0.58 (t, J=7.6 Hz, 3H).

### Synthesis of 33

A solution of 33.3 (100 mg, 0.24 mmol) and K₂CO₃ (64.8 mg, 0.47 mmol) and 1H-pyrazole-4-carbonitrile (43.7 mg, 0.47 mmol) in acetone (10 mL) was stirred at RT for 16 h. The mixture was poured into water (50 mL) then extracted with EtOAc (3×20 mL). The combined organic layer was washed with water (100 mL), saturated brine (100 mL), dried over anhydrous Na₂SO₄, filtered and concentrated. The residue was purified by flash column (0-30% of EtOAc in PE) to give 33 (22.0 mg, 22%). ¹H NMR (400 MHz, CDCl₃)^{6H} 7.86 (s, 1H), 7.80 (s, 1H), 5.16 (d, J=18 Hz, 1H), 4.92 (d, J=18 Hz, 1H), 2.49 (t, J=8.4 Hz, 1H), 2.40-2.20 (m, 2H), 1.90-1.59 (m, 10H), 1.52-1.22 (m, 13H), 1.20-0.85 (m, 6H), 0.61 (t, J=7.6 Hz, 3H). LC-ELSD/MS 30-90AB-2 min-E, purity 99%, MS ESI calcd. for C₂₇H₃₈N₃₀ [M−H₂O+H]⁺ 420.3, found 420.3.

### Example 34 & 35: Synthesis of 1-((1S,3aS,3bS,8S,10aR,10bS,12aS)-12a-ethyl-8-hydroxy-8,10a-dimethyl-1,2,3,3a,3b,4,6,7,8,9,10,10a,10b,11,12,12a-hexadecahydrocyclohepta[a]cyclopenta[f]naphthalen-1-yl)ethanone (34)& 1-(2-((1S,3aS,3bS,8S,10aR,10bS,12aS)-12a-ethyl-8-hydroxy-8,10a-dimethyl-1,2,3,3a,3b,4,6,7,8,9,10,10a,10b,11,12,12a-hexadecahydrocyclohepta[a]cyclopenta[f]naphthalen-1-yl)-2-oxoethyl)-1H-pyrazole-4-carbonitrile (35)

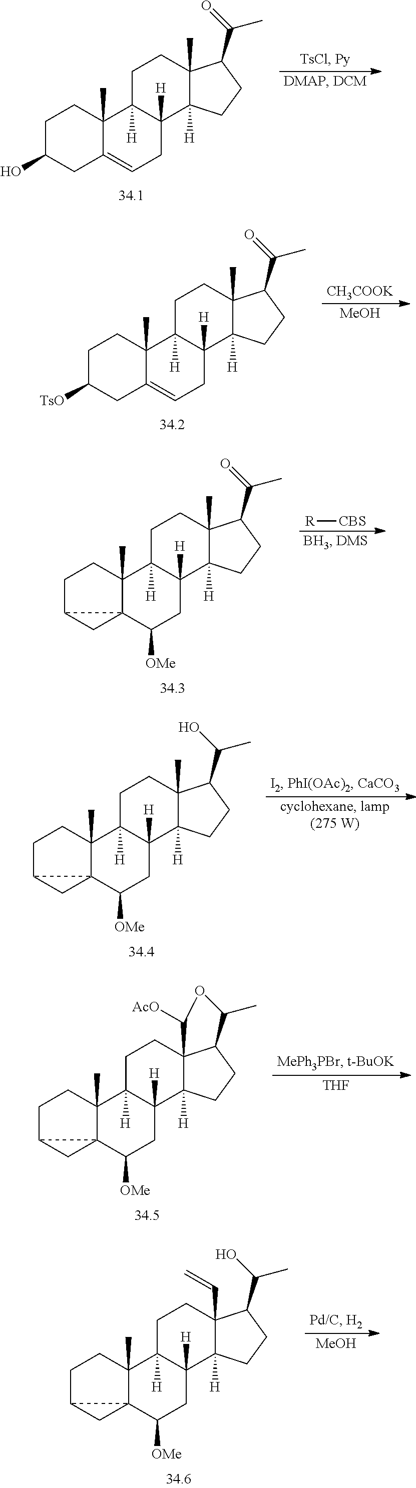

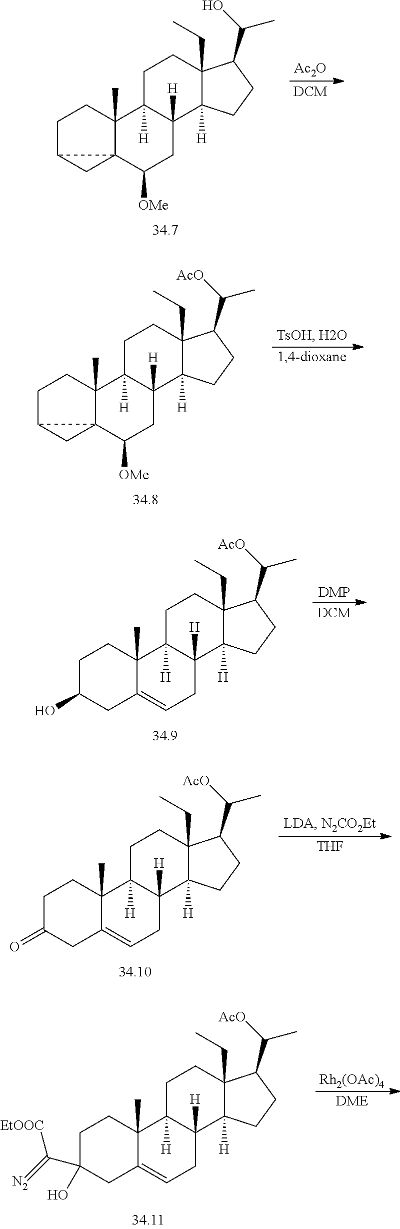

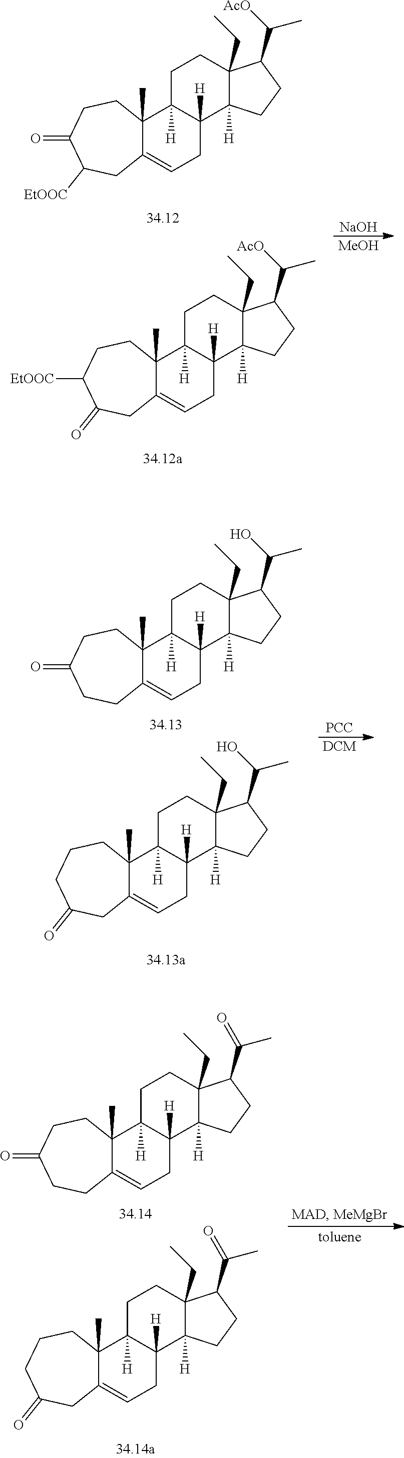

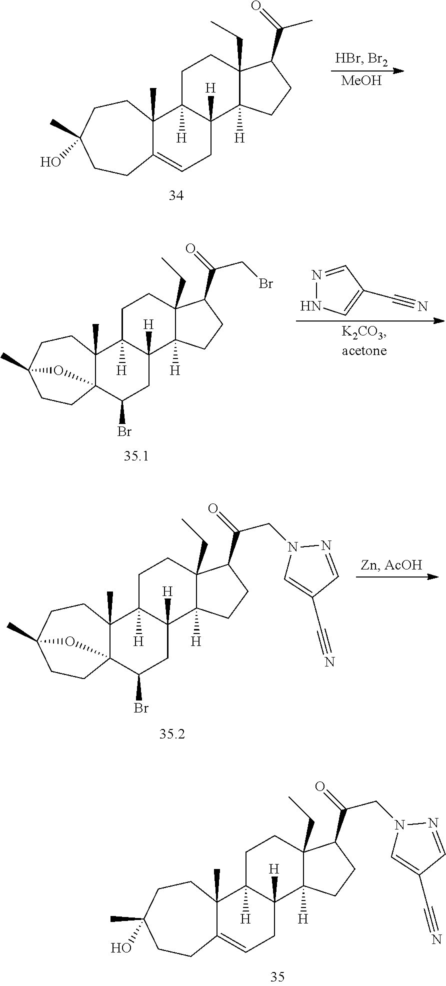

### Synthesis of 34.2

To a solution of 34.1 (50.0 g, 157 mmol) in pyridine (400 ml) was added TsCl (89.6 g, 470 mmol) and DMAP (8.59 g, 78.5 mmol), and the mixture was stirred at RT for 12 h. Water (200 mL) was added and the suspension was filtered. The filter cake was washed with water (200 mL) and dried to get 34.2 (72.0 g, 98%). ¹H NMR (400 MHz, CDCl₃) δ_{H}7.81-7.77 (m, 2H), 7.35-7.31 (m, 2H), 5.32-5.28 (m, 1H), 4.38-4.28 (m, 1H), 2.55-2.49 (m, 1H), 2.48-2.39 (m, 4H), 2.31-2.24 (m, 1H), 2.21-2.13 (m, 1H), 2.11 (s, 3H), 2.05-1.93 (m, 2H), 1.86-1.79 (m, 2H), 1.75-1.63 (m, 3H), 1.60-1.50 (m, 2H), 1.49-1.39 (m, 3H), 1.27-1.01 (m, 3H), 098-0.85 (m, 4H), 0.61 (s, 3H).

### Synthesis of 34.3

To a solution of KOAc (82.0 g, 836 mmol) in MeOH (700 mL) was added 34.2 (72.0 g, 152 mmol). The mixture was heated at 80° C. for 16 h. After cooling, the mixture was poured into water (500 mL) and the suspension was filtered. The filter cake was washed with water (100 mL) and dried to afford 34.3 (50.0 g, crude). ¹H NMR (400 MHz, CDCl₃) δ_{H}2.23-2.11 (m, 4H), 2.06-2.00 (m, 1H), 1.94-1.87 (m, 1H), 1.82-1.60 (m, 4H), 1.54-1.40 (m, 4H), 1.36-1.06 (m, 4H), 1.02 (s, 3H), 0.93-0.80 (m, 3H), 0.69-0.63 (m, 4H), 0.48-0.42 (m, 1H).

### Synthesis of 34.4

At 0° C., to a solution of (R)—CBS (3.35 g, 12.1 mmol) in toluene (20 mL) was added a solution of BH₃-Me₂S (36.3 mL, 10 M, 363 mmol) in toluene (80 mL). The mixture was stirred at 0° C. for 30 min and a solution of 34.3 (40.0 g, 121 mmol) in toluene (100 mL) was added dropwise. The mixture was stirred at 0° C. for another 1 h, and MeOH (30 mL) was added dropwise. The mixture was concentrated and purified by CombiFlash (10% of EtOAc in PE) to give 34.4 (32.0 g, 80%). ¹H NMR (400 MHz, CDCl₃) δ_{H} 3.75-3.67 (m, 1H), 3.32 (s, 3H), 2.79-2.76 (m, 1H), 1.96-1.85 (m, 3H), 1.80-1.65 (m, 3H), 1.57-1.46 (m, 3H), 1.45-1.31 (m, 3H), 1.25-1.21 (m, 4H), 1.19-1.04 (m, 4H), 1.02 (s, 3H), 0.92-0.79 (m, 3H), 0.72 (s, 3H), 0.67-0.63 (m, 1H), 0.46-0.40 (m, 1H).

### Synthesis of 34.5

To a solution of 34.4 (2.00 g, 6.01 mmol) in cyclohexane (200 mL) were added CaCO₃ (1.80 g, 18.0 mmol), PhI(OAc)₂ (5.79 g, 18.0 mmol), I₂ (3.04 g, 12.0 mmol). The mixture was heated to reflux by irradiation with infrared lamp (275 W) for 30 min. The reaction was allowed to cool to RT and quenched with aqueous Na₂S₂O₃ (100 mL). The suspension was extracted with EtOAc (2×50 mL). The combined organic layer was washed with brine (50 mL), dried over Na₂SO₄, filtered and concentrated to give 34.5 (2.33 g, crude), which was used directly without further purification.

### Synthesis of 34.6

To a mixture of MePPh₃Br (17.1 g, 47.9 mmol) in THF (70 mL) was added t-BuOK (5.37 g, 47.9 mmol). The resulting mixture was stirred at 50° C. for 30 min and 34.5 (2.33 g, 5.99 mmol) in THE (10 mL) was added in small portions. After stirring at 50° C. for 12 h, the reaction mixture was poured into water (100 mL) then extracted with EtOAc (2×100 mL). The combined organic layer was washed with water (30 mL), brine (30 mL), dried over Na₂SO₄, filtered and concentrated. The residue was purified by silica gel chromatography (0-12% EtOAc in PE) to get 34.6 (0.75 g, 36%). ¹H NMR (400 MHz, CDCl₃) δ_{H} 5.90-5.78 (m, 1H), 5.32 (dd, J=1.2, 11.2 Hz, 1H), 5.17 (dd, J=1.6, 18.0 Hz, 1H), 3.89-3.79 (m, 1H), 3.31 (s, 3H), 2.76 (t, J=3.2 Hz, 1H), 2.42-2.34 (m, 1H), 1.93-1.88 (m, 1H), 1.85-1.70 (m, 5H), 1.68-1.60 (m, 1H), 1.56-1.44 (m, 5H), 1.43-1.36 (m, 1H), 1.33-1.27 (m, 1H), 1.15-1.04 (m, 5H), 0.94 (s, 3H), 0.91-0.81 (m, 3H), 0.66-0.62 (m, 1H), 0.45-0.40 (m, 1H).

### Synthesis of 34.7

To a solution of 34.6 (9.00 g, 26.1 mmol) in MeOH (100 mL) was added Pd/C (0.60 g, 10% Palladium on carbon, 50% water wet). The mixture was hydrogenated under 15 psi of hydrogen at RT for 72 h. The reaction mixture was filtered through a pad of Celite and washed with MeOH (3×40 mL). The filtrate was concentrated to afford 34.7 (8.10 g, 90%).

¹H NMR (400 MHz, CDCl₃) δ_{H} 3.94-3.78 (m, 1H), 3.34 (s, 3H), 2.80 (t, J=2.8 Hz, 1H), 2.22-2.14 (m, 1H), 1.95-1.73 (m, 4H), 1.72-1.61 (m, 2H), 1.57-1.34 (m, 5H), 1.29 (s, 5H), 1.23-1.07 (m, 4H), 1.04 (s, 3H), 0.99-0.93 (m, 2H), 0.92-0.85 (m, 5H), 0.69-0.65 (m, 1H), 0.48-0.43 (m, 1H).

### Synthesis of 34.8

To a solution of 34.7 (5.00 g, 14.4 mmol) in DCM (100 mL) was added DMAP (1.75 g, 14.4 mmol) and Ac₂O (5.87 g, 57.6 mmol). After stirring at RT for 16 h, the reaction mixture was poured into water (80 mL) and stirred for 10 min. The suspension was extracted with DCM (2×60 mL) and the combined organic phase was washed with saturated brine (2×50 mL), dried over anhydrous Na₂SO₄, filtered and concentrated. The residue was purified by flash column (0˜15% of EtOAc in PE) to give 34.8 (5.40 g, 97%). ¹H NMR (400 MHz, CDCl₃) δ_{H} 5.09-4.96 (m, 1H), 3.31 (s, 3H), 2.79-2.75 (m, 1H), 2.17-2.11 (m, 1H), 2.00 (s, 3H), 1.92-1.86 (m, 1H), 1.84-1.70 (m, 3H), 1.65-1.59 (m, 1H), 1.56-1.47 (m, 4H), 1.44-1.41 (m, 1H), 1.29-1.25 (m, J=6.0 Hz, 4H), 1.24-1.08 (m, 4H), 1.01 (s, 3H), 0.97-0.88 (m, 3H), 0.87-0.81 (m, 5H), 0.66-0.62 (m, 1H), 0.46-0.41 (m, 1H).

### Synthesis of 34.9

To a solution of 34.8 (5.40 g, 13.8 mmol) in 1,4-dioxane (60 mL) was added a solution of TsOH (237 mg, 1.38 mmol) in water (15 mL). After stirring at 75° C. for 16 h, the mixture was poured into saturated NaHCO₃ (200 mL). The suspension was extracted with DCM (3×80 mL) and the combined organic layer was washed with saturated brine (50 mL), dried over anhydrous Na₂SO₄, filtered and concentrated. The residue was purified by flash column (0-30% of EtOAc in PE) to give 34.9 (4.50 g, 87%). ¹H NMR (400 MHz, CDCl₃) δ_{H} 5.37-5.33 (m, 1H), 5.07-4.97 (m, 1H), 3.58-3.47 (m, 1H), 2.34-2.12 (m, 3H), 2.04-1.94 (m, 4H), 1.88-1.78 (m, 3H), 1.65-1.50 (m, 8H), 1.36-1.23 (m, 6H), 1.15-1.02 (m, 3H), 0.99 (s, 3H), 0.98-0.90 (m, 2H), 0.85 (t, J=7.6 Hz, 3H).

### Synthesis of 34.10

To a solution of 34.9 (3.50 g, 9.34 mmol) in DCM (70 mL) was added Dess-Martin Reagent (9.88 g, 23.2 mmol) and the mixture was stirred at 40° C. for 4 h. Saturated NaHCO₃ aqueous solution (40 mL) was added and the suspension extracted with DCM (3×40 mL). The combined organic phase was washed with saturated NaHCO₃/Na₂S₂O₃ (1:1, 2×100 mL), brine (30 mL), dried over Na₂SO₄, filtered and concentrated to give 34.10 (3.47 g, crude), which was used for the next step without further purification. ¹H NMR (400 MHz, CDCl₃) δ_{H} 5.37-5.30 (m, 1H), 5.07-4.95 (m, 2H), 3.32-3.19 (m, 1H), 3.32-3.19 (m, 1H), 2.86-2.79 (m, 1H), 2.62-2.43 (m, 2H), 2.35-2.24 (m, 2H), 2.21-2.07 (m, 4H), 1.87-1.78 (m, 4H), 1.55-1.46 (m, 4H), 1.36-1.26 (m, 11H), 0.88-0.85 (m, 3H).

### Synthesis of 34.11

At −70° C., an LDA (46.5 mmol in THF 40 mL) solution was added to a stirred solution of 34.10 (3.47 g, 9.31 mmol) and ethyl diazoacetate (5.88 g, 46.5 mmol, 90%) in THF (80 mL). After stirring at −70° C. for 2 h, HOAc (2.65 mL, 15 mmol) in THE (15 mL) was added, and the mixture was warmed to 20° C. for 16 hours. Water (30 mL) was added and the suspension was extracted with EtOAc (3×60 mL). The combined organic layers were washed with brine (30 mL), dried over anhydrous Na₂SO₄, filtered and concentrated. The residue was purified by flash column (0˜15% of EtOAc in PE) to give 34.11 (1.30 g, crude).

¹H NMR (400 MHz, CDCl₃) δ_{H} 5.45-5.22 (m, 1H), 5.07-4.98 (m, 1H), 4.35-4.18 (m, 2H), 3.69-3.04 (m, 1H), 2.69-2.57 (m, 1H), 2.44-2.34 (m, 1H), 2.32-2.24 (m, 1H), 2.20-2.10 (m, 1H), 2.03-1.95 (m, 4H), 1.92-1.67 (m, 4H), 1.58-1.46 (m, 5H), 1.37-1.23 (m, 10H), 1.14-0.91 (m, 7H), 0.85 (t, J=7.6 Hz, 3H).

### Synthesis of 34.12 &3 4.12a

To a solution of 34.11 (1.30 g, crude) in DME (50 mL) was added Rh₂(OAc)₄ (80 mg, 0.18 mmol) in one portion. The mixture was stirred at 30° C. for 12 h. The mixture was treated with H₂O (40 mL) then extracted with EtOAc (3×60 mL). The combined organic phase was washed with brine (2×30 mL), dried over anhydrous Na₂SO₄, filtered and concentrated to afford a mixture of 34.12 & 34.12a (1.20 g, crude) ¹H NMR (400 MHz, CDCl₃) δ_{H} 12.79-12.59 (m, 1H), 5.66-5.53 (m, 1H), 5.07-4.97 (m, 1H), 4.27-4.04 (m, 2H), 3.57-3.14 (m, 1H), 3.02-2.60 (m, 1H), 2.50-2.07 (m, 3H), 2.04-1.97 (m, 4H), 1.89-1.67 (m, 3H), 1.66-1.61 (m, 1H), 1.56-1.39 (m, 6H), 1.33-1.23 (m, 9H), 1.18-1.08 (m, 3H), 1.01-0.94 (m, 3H), 0.90-0.81 (m, 3H).

### Synthesis of 34.13 & 34.13a

To a solution of 34.12 & 34.12a (1.20 g, 2.61 mmol) in MeOH (20 mL) and H₂O (5 mL) and THF (10 mL) was added NaOH (623 mg, 15.6 mmol). The reaction was stirred at 65° C. for 1 h, then poured into saturated brine (100 mL). The resultant suspension was extracted with EtOAc (3×100 mL), the combined organic layer was washed with HCl (1M, 100 mL), saturated NaHCO₃ (100 mL) and brine (100 mL). The organic solution was dried over anhydrous Na₂SO₄, filtered and concentrated. The residue was purified by flash column (0˜35% of EtOAc in PE) to give a mixture of 34.13 & 34.13a (430 mg, 52.7%). ¹H NMR (400 MHz, CDCl₃) δ_{H} 5.64-5.51 (m, 1H), 3.91-3.81 (m, 1H), 3.31-2.77 (m, 1H), 2.65-2.54 (m, 1H), 2.44-2.28 (m, 2H), 2.23-2.12 (m, 2H), 2.04 (s, 1H), 1.95-1.86 (m, 1H), 1.76-1.69 (m, 1H), 1.62-1.50 (m, 6H), 1.42-1.07 (m, 12H), 1.03-0.93 (m, 4H), 0.90-0.80 (m, 3H) Synthesis of 34.14 & 34.14a

At 0° C., to a solution of 34.13&34.13a (480 mg, 1.39 mmol) in DCM (30 mL) was added silica gel (600 mg) and PCC (599 mg, 2.78 mmol) and the mixture was stirred at RT for 2 h. The mixture was concentrated then purified by flash column (0%-20% of EtOAc in PE) to give 34.14 & 34.14a (420 mg, mixture), which was separated by SFC (column: DAICEL CHIRALPAK AD (250 mm*30 mm*10 um), A; CO₂; B: 0.1% NH₃H₂O EtOH; gradient: 30-30%, flow rate: 70 mL/min) to afford 34.14 (160 mg, 38%) and 34.14a (200 mg, 48%).

34.14: ¹H NMR (400 MHz, CDCl₃) δ_{H} 5.60-5.55 (m, 1H), 2.66-2.58 (m, 1H), 2.49-2.41 (m, 2H), 2.40-2.23 (m, 5H), 2.22-2.13 (m, 4H), 2.09-1.99 (m, 1H), 1.78-1.72 (m, 2H), 1.67-1.60 (m, 4H), 1.58-1.56 (m, 1H), 1.36-1.18 (m, 7H), 0.99 (s, 3H), 0.65 (t, J=7.6 Hz, 3H).

34.14a: ¹H NMR (400 MHz, CDCl₃) δ_{H} 5.60-5.52 (m, 1H), 3.29-3.20 (m, 1H), 2.87-2.78 (m, 1H), 2.64-2.56 (m, 1H), 2.50-2.43 (m, 1H), 2.39-2.33 (m, 1H), 2.30-2.17 (m, 5H), 2.13-2.03 (m, 1H), 1.90-1.83 (m, 1H), 1.71-1.58 (m, 6H), 1.56-1.50 (m, 2H), 1.35-1.18 (m, 7H), 0.98 (s, 3H), 0.66 (t, J=7.6 Hz, 3H).

### Synthesis of 34

At −70° C., to a freshly prepared MAD (2.33 mmol in 2.5 mL toluene) solution was added a solution of 34.14 (200 mg, 0.58 mmol) in DCM (2 mL) dropwise. After stirring at −70° C. for 1 h, MeMgBr (0.776 mL, 2.33 mmol, 3 M in ethyl ether) was added dropwise and the resulting solution was stirred at −70° C. for another 5 h. The reaction mixture was poured into saturated aqueous citric acid (20 mL) and extracted with EtOAc (2×30 mL). The combined organic layer was dried over Na₂SO₄, filtered and concentrated. The residue was purified by flash column (0˜30% of EtOAc in PE) to give 34 (160 mg, 77%). ¹H NMR (400 MHz, CDCl₃) δ_{H} 5.49-5.43 (m, 1H), 2.50-2.42 (m, 1H), 2.37-2.20 (m, 5H), 2.08-1.86 (m, 4H), 1.77-1.62 (m, 4H), 1.60-1.52 (m, 2H), 1.51-1.37 (m, 3H), 1.34-1.17 (m, 12H), 0.87 (s, 3H), 0.64 (t, J=7.6 Hz, 3H). LC-ELSD/MS: purity 99%; MS ESI calcd. for C₂₄H₃₇O [M−H₂O+H]⁻ 341.3, found 341.3.

### Synthesis of 35.1

At 0° C., to a solution of 34 (130 mg, 0.36 mmol) and HBr (3.00 mg, 0.01 mmol, 40%) in MeOH (10 mL) was added Br₂ (144 mg, 0.91 mmol). The mixture was stirred at RT for 2 h then poured into saturated NaHCO₃ (20 mL). The suspension was extracted with EtOAc (3×20 mL) and the combined organic layer was washed with saturated brine (30 mL), dried over anhydrous Na₂SO₄, filtered and concentrated to give 35.1 (200 mg, crude). ¹H NMR (400 MHz, CDCl₃) δ 4.10-3.97 (m, 3H), 2.77 (t, J=8.8 Hz, 1H), 2.29-2.23 (m, 2H), 1.97-1.88 (m, 3H), 1.70-1.62 (m, 5H), 1.46-1.38 (m, 3H), 1.29-1.22 (m, 10H), 1.15-1.11 (m, 4H), 0.91-0.83 (m, 2H), 0.61 (t, J=7.6 Hz, 3H).

### Synthesis of 35.2

To a solution of 35.1 (200 mg, 0.39 mmol) in acetone (10 mL) was added K₂CO₃ (106 mg, 0.77 mmol) and 1H-pyrazole-4-carbonitrile (43.2 mg, 0.46 mmol). The reaction mixture was stirred at RT for 2 h to give a yellow mixture. The reaction was quenched with saturated aqueous NH₄Cl solution (30 mL) and extracted with EtOAc (2×30 mL). The combined organic phase was washed with saturated brine (50 mL), dried over anhydrous Na₂SO₄, filtered and concentrated. The residue was purified by silica gel chromatography (0-45% of EtOAc in PE) to give the product 35.2 (100 mg, 49.0%). ¹H NMR (400 MHz, CDCl₃) δ 7.87 (s, 1H), 7.81 (s, 1H), 5.16 (d, J=18.0 Hz, 1H), 4.93 (d, J=18.0 Hz, 1H), 4.08 (d, J=2.4 Hz, 1H), 2.55-2.46 (m, 1H), 2.41-2.20 (m, 3H), 2.10-1.85 (m, 4H), 1.76-1.63 (m, 5H), 1.50-1.40 (m, 2H), 1.34-1.22 (m, 8H), 1.19-1.11 (m, 4H), 0.90-0.83 (m, 3H), 0.63 (t, J=7.6 Hz, 3H).

### Synthesis of 35

To a solution of 35.2 (100 mg, 0.19 mmol) in AcOH (20 mL) and water (1 mL) was added Zinc (2.47 g, 37.8 mmol). The reaction mixture was stirred for 16 h at RT to give a white suspension. The reaction mixture was poured into saturated aqueous NH₄Cl (50 mL), then extracted with EtOAc (3×20 mL). The combined organic layer was washed with saturated brine (80 mL), dried over anhydrous Na₂SO₄, filtered and concentrated. The residue was purified by silica gel chromatography to give product 35 (60.0 mg, crude), which was further purified by prep-HPLC (Column: YMC Triart C18 150*25 mm*5 um; Condition: water (10 mM NH₄HCO₃)-ACN; Begin B: 72; End B: 100; Gradient Time (min): 9.5; 100% B Hold Time (min): 2) to give the pure product 35 (43.5 mg, 51.1%). ¹H NMR (400 MHz, CDCl₃) δ 7.87 (s, 1H), 7.81 (s, 1H), 5.47 (d, J=3.2 Hz, 1H), 5.17 (d, J=18.4 Hz, 1H), 4.93 (d, J=18.4 Hz, 1H), 2.53-2.46 (m, 1H), 2.41-2.26 (m, 2H), 2.09-1.84 (m, 4H), 1.77-1.60 (m, 6H), 1.52-1.36 (m, 4H), 1.33-1.20 (m, 11H), 0.88 (s, 3H), 0.63 (t, J=7.6 Hz, 3H). LC-ELSD/MS: purity 99%; MS ESI calcd. for C₂₈H₃₈N₃O [M+H-H₂O]⁺432.3, found 432.3.

### Example 36: Synthesis of 1-((1R,4aS,4bR,6aR,8R,11aS,11bR,13aS)-8-ethyl-8-hydroxy-13a-methyloctadecahydro-1H-cyclohepta[a]phenanthren-1-yl)ethenone (36)

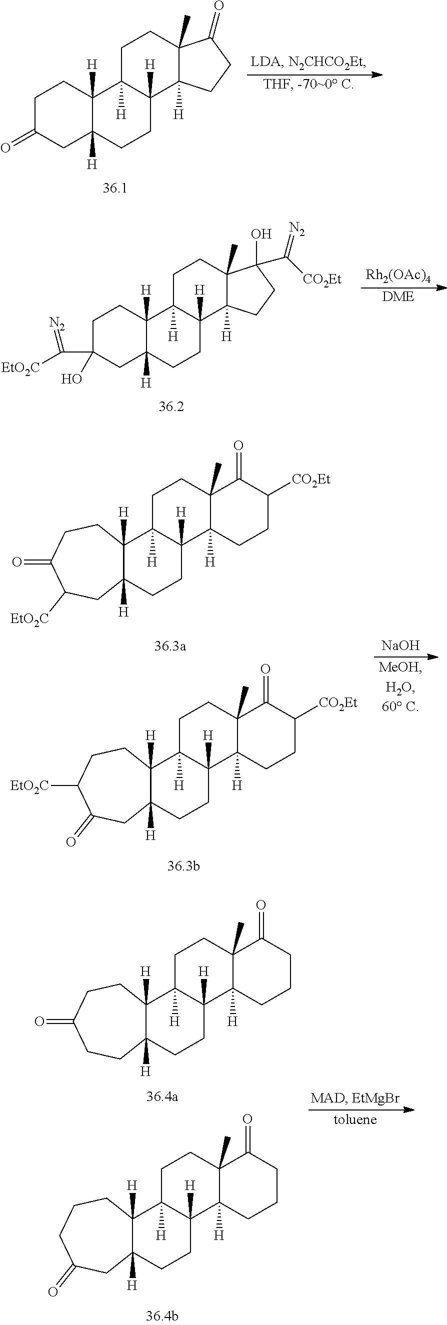

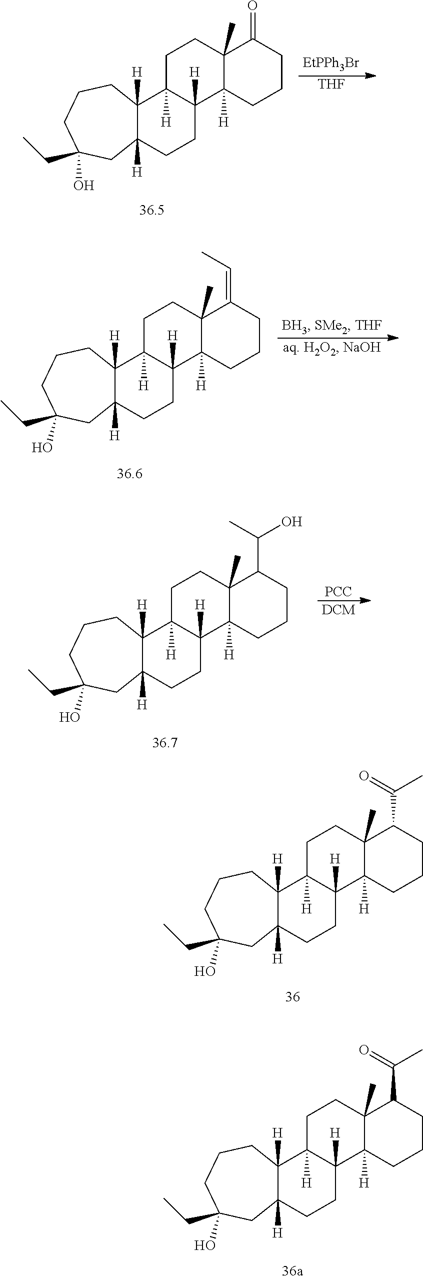

### Synthesis of 36.2

A cooled (−78° C.) LDA solution (145 mmol, 1 M in THF) was added to a stirred solution of 36.1 (5.0 g, 18.2 mmol) and ethyl diazoacetate (16.5 g, 145 mmol) in THF (150 mL) at −70° C. The mixture was stirred at −70° C. for 2 h. HOAc (8.70 g, 145 mmol) in TF (50 mL) was added, and the mixture was warmed to 25° C. and stirred for 16 h. Water (300 mL) was added and the suspension was extracted with EtOAc (3×150 mL). The combined organic layers were washed with saturated brine (2×200 mL), dried over anhydrous Na₂SO₄, filtered and concentrated to give the product 36.2 (20.0 g, crude), which was used directly to next step.

### Synthesis of 36.3a & 36.3b

To a solution of 36.2 (20.0 g, 39.7 mmol) in DME (150 mL) was added Rh₂(OAc)₄ (350 mg, 0.794 mmol). The reaction mixture was stirred at RT for 16 h to give a brown solution. The reaction mixture was concentrated, and the residue was purified by silica gel chromatography (0-20% of EtOAc in PE) to give the crude mixture product 36.3a & 36.3b (11.0 g, impure).

### Synthesis of 36.4a & 36.4b

To a mixture of 36.3a & 36.3b (11.0 g, 24.6 mmol) in MeOH (100 mL) was added H₂O (50 mL) and NaOH (7.84 g, 196 mmol). The reaction mixture was stirred at 60° C. for 16 h to give a yellow mixture. After cooling, H₂O (200 mL) was added and the mixture was extracted with EtOAc (2×200 mL). The combined organic phase was washed with saturated brine (200 mL), dried over anhydrous Na₂SO₄, filtered and concentrated. The residue was purified by flash column (0˜30% of EtOAc in PE) to give a mixture of 36.4a & 36.4b (2.6 g), which was separated by SFC (column: DAICEL CHIRALCEL OJ (250 mm*50 mm*10 um); Mobile phase: A: CO₂ B: 0.1% NH₃H₂O EtOH; gradient: from 20% to 20% of B, Flow rate (ml/min): 180) to give 36.4a (1.0 g, 13.4%) and 36.4b (1.0 g, 13.4%).

36.4b: ¹H NMR (400 MHz, CDCl₃) δ ppm 3.11-2.86 (m, 1H), 2.70-2.55 (m, 1H), 2.53-2.31 (m, 2H), 2.26-2.15 (m, 1H), 2.10-1.91 (m, 4H), 1.85-1.48 (m, 12H), 1.43-1.17 (m, 5H), 1.10 (s, 3H), 0.92-0.84 (m, 1H). LC-ELSD/MS purity 99%, anlytic SFC: 100% de; MS ESI calcd. for C₂₀H₃₀O₂[M+H]⁺ 303.2, found 303.2.

36.4a: ¹H NMR (400 MHz, CDCl₃) δ ppm 2.68-2.38 (m, 4H), 2.36-2.25 (m, 1H), 2.24-2.14 (m, 1H), 2.11-1.58 (m, 13H), 1.56-1.13 (m, 6H), 1.10 (s, 3H), 1.09-0.85 (m, 2H). LC-ELSD/MS purity 99%, anlytic SFC: 100% de; MS ESI calcd. for C₂₀H₃₀O₂[M−H₂O+H]⁺ 285.2, found 285.2.

### Synthesis of 36.5

At −70° C., to a freshly prepared MAD (14.8 mmol, in 15 mL toluene) solution was added 36.4b (1.50 g, 4.95 mmol) in DCM (15 mL) dropwise. After stirring at −70° C. for 1 h under, EtMgBr (6.60 mL, 19.8 mmol, 3M in ethyl ether) was added dropwise and the resulting solution was stirred at −70° C. for another 4 h. The reaction mixture was poured into citric acid (50 mL, 20%) and extracted with EtOAc (2×50 mL). The combined organic layer was dried over Na₂SO₄, filtered and concentrated. The residue was purified by silica gel chromatography (0-25% of EtOAc in PE) to give 36.5 (300 mg, 18.2%).

36.5: ¹H NMR (400 MHz, CDCl₃) δ_{H} 2.66-2.56 (m, 1H), 2.45-2.35 (m, 1H), 2.25-2.15 (m, 1H), 2.10-1.98 (m, 2H), 1.86-1.66 (m, 4H), 1.65-1.55 (m, 5H), 1.55-1.12 (m, 12H), 1.10 (s, 3H), 1.05 (s, 1H), 1.03-0.94 (m, 1H), 0.90 (t, J=7.2 Hz, 3H), 0.86-0.74 (m, 2H). LC-ELSD/MS purity 99%, MS ESI calcd. for C₂₂H₃₅O [M+H-H₂O]⁺ 315.3, found 315.3.

### Synthesis of 36.6

To a mixture of EtPPh₃Br (4.19 g, 11.3 mmol) in THE (11 mL) was added t-BuOK (1.26 g, 11.3 mmol) and the resulting mixture was stirred at 40° C. for 1 h. 36.5 (630 mg, 1.89 mmol) was added in small portions and the reaction mixture was stirred at 65° C. for 16 h. After cooling, the reaction was quenched with 10% NH₄Cl aqueous (50 mL). The resultant suspension was extracted with EtOAc (2×50 mL) and the combined organic phase was concentrated. The residue was purified by flash column (0˜10% of EtOAc in PE) to give 36.6 (380 mg, 58.3%). ¹H NMR (400 MHz, CDCl₃) δ_{H} 5.1-5.11 (m, 1H), 2.54-2.46 (m, 1H), 2.27-2.07 (m, 1H), 1.96-1.68 (m, 10H), 1.61-1.56 (m, 9H), 1.51-1.30 (m, 7H), 1.19-0.99 (m, 5H), 0.94-0.88 (m, 6H).

### Synthesis of 36.7

To a solution of 36.6 (380 mg, 1.1 mmol) in THE (5 mL) was added BH₃-Me₂S (990 μL, 10 M, 9.90 mmol) and the mixture was stirred at 15° C. for 16 h. To the mixture was added EtOH (3.16 mL, 55.0 mmol) dropwise followed by NaOH (2.20 g in 11 mL water, 5 M, 55.0 mmol) and H₂O₂(5.5 mL, 10 M, 55.0 mmol). The mixture was the heated and stirred at 78° C. for 2 h. The reaction was cooled and quenched with Na₂S₂SO₃ (30 mL, 10%). The suspension was extracted with EtOAc (2×20 mL). The combined organic layer was dried over Na₂SO₄, filtered and concentrated to give 36.7 (272 mg, crude). ¹H NMR (400 MHz, CDCl₃) δ_{H} 4.34-4.03 (m, 1H), 1.93-1.53 (m, 13H), 1.53-1.37 (m, 5H), 1.36-1.20 (m, 7H), 1.18-1.02 (m, 5H), 1.01-0.98 (m, 2H), 0.97-0.87 (m, 6H), 0.86-0.81 (m, 1H), 0.78-0.75 (m, 2H).

### Synthesis of 36 & 36a

At 0° C., to a solution of 36.7 (170 mg, 468 μmol) in DCM (6 mL) was added silica gel (201 mg) and PCC (201 mg, 936 mol). The mixture was stirred at 10° C. for 0.5 h, then PE (3 mL) was added. The resulting mixture was filtered through a pad of silica gel and the filter cake was washed with DCM (3×6 mL). The filtrate was concentrated, and the residue was purified by silica gel chromatography (0-20% of EtOAc in PE) to give the 36 (65.0 mg, 38.6%) and 36a (65.0 mg, 38.6%).

36: ¹H NMR (400 MHz, CDCl₃) δ_{H} 2.49-2.45 (m, 1H), 2.13 (s, 3H), 1.85-1.76 (m, 4H), 1.75-1.69 (m, 3H), 1.69-1.56 (m, 5H), 1.51-1.39 (m, 6H), 1.34-1.18 (m, 6H), 1.15-1.02 (m, 3H), 0.93 (s, 3H), 0.92-0.83 (m, 6H). LC-ELSD/MIS purity 99%, MS ESI calcd. for C₂₂H₃₉O [M+H-H₂O]343.3, found 343.3.

36a: ¹H NMR (400 MHz, CDCl₃)⁶H 2.34-2.26 (m, 1H), 2.14 (s, 3H), 1.86-1.78 (m, 3H), 1.76-1.65 (m, 5H), 1.62-1.56 (m, 3H), 1.51-1.38 (m, 5H), 1.35-1.20 (m, 6H), 1.15-1.11 (m, 1H), 1.06-0.94 (m, 4H), 0.94-0.90 (m, 6H), 0.89-0.80 (m, 3H). LC-ELSD/MS purity 99%, MS ESI calcd. for C₂₂H₃₉O [M+H-H₂O]⁺343.3, found 343.3.

### Example 37 & 38 & 39: Synthesis of 1-((1S,4aS,4bR,6aR,8R,11aS,11bS,13aS)-8-ethyl-8-hydroxy-11a,13a-dimethyloctadecahydro-1H-cyclohepta[a]phenanthren-1-yl)ethanone (37) & Synthesis of 1-(2-((1S,4aS,4bR,6aR,8R,11aS,11bS,13aS)-8-ethyl-8-hydroxy-11a,13a-dimethyloctadecahydro-1H-cyclohepta[a]phenanthren-1-yl)-2-oxoethyl)-1H-pyrazole-4-carbonitrile (38) & Synthesis of 1-(2-((1R,4aS,4bR,6aR,8R,11aS,11bS,13aS)-8-ethyl-8-hydroxy-11a,13a-dimethyloctadecahydro-1H-cyclohepta[a]phenanthren-1-yl)-2-oxoethyl)-1H-pyrazole-4-carbonitrile (39)

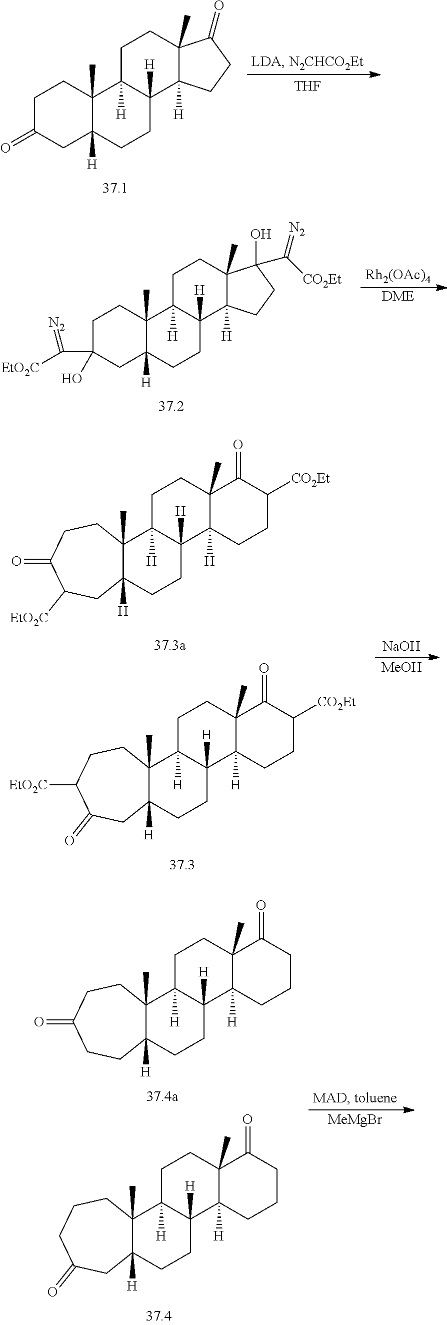

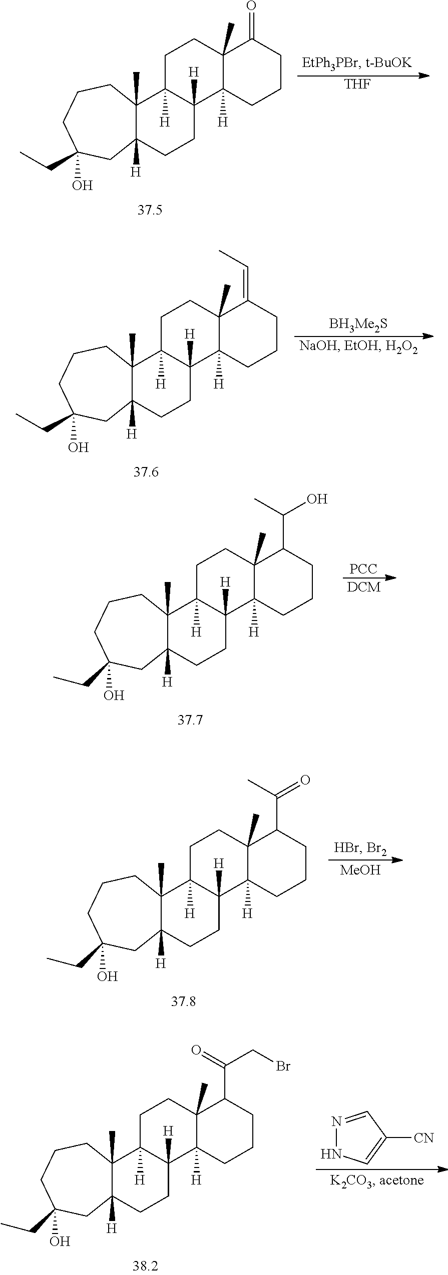

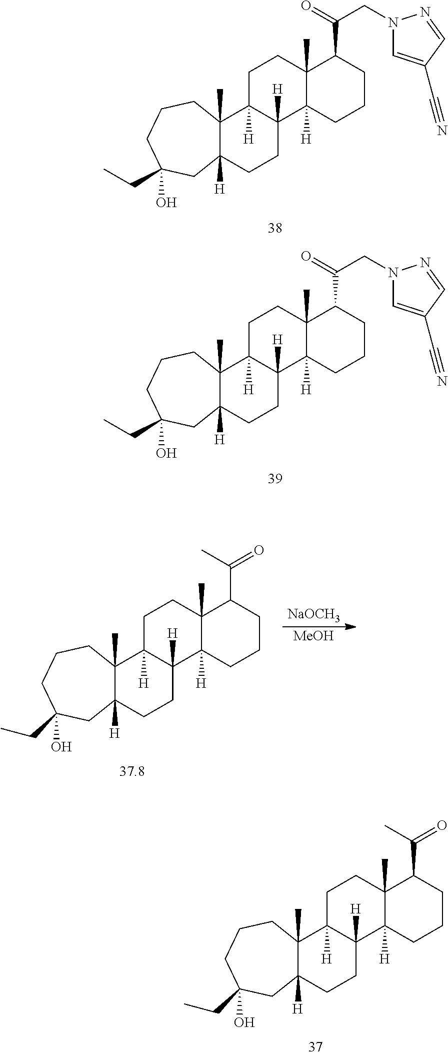

### Synthesis of 37.2

A cooled (−70° C.) LDA solution (138 mmol) was added to a stirred solution of 37.1 (5.00 g, 17.3 mmol) and ethyl diazoacetate (15.7 g, 138 mmol) in THF (300 mL) at −70° C. After stirring at −70° C. for 2 h, HOAc (8.28 g, 138 mmol) in THF (50 mL) was added. The mixture was then warm to RT and stirred for another 16 h. Water (800 mL) and PE (200 mL) was added and the layers were separated. The aqueous phase was extracted with EtOAc (300 mL). The combined organic layers were washed with saturated brine (1000 mL), dried over anhydrous Na₂SO₄, filtered and concentrated to give the product 37.2 (9.50 g, crude), which was used directly to the next step.

### Synthesis of 37.3 & 37.3a

To a solution of 37.2 (9.50 g, 18.3 mmol) in DME (150 mL) was added Rh₂(OAc)₄ (161 mg, 0.37 mmol). The reaction mixture was stirred at RT for 16 h to give a brown solution. The reaction mixture was concentrated, and the residue was purified by silica gel chromatography (0-20% of EtOAc in PE) to give the mixture product 37.3 & 37.3a (8.50 g, impure).

### Synthesis of 37.4 & 37.4a

To a mixture of 37.3 & 37.3a (8.00 g, 17.3 mmol) in MeOH (160 mL) was added H₂O (60 mL) and NaOH (5.52 g, 138 mmol). The reaction mixture was stirred at 60° C. for 16 h to give a yellow mixture. The reaction mixture was concentrated, H₂O (200 mL) and EtOAc (200 mL) were added. The layers were separated, and the aqueous layer was extracted with EtOAc (200 mL). The combined organic phase was washed with saturated brine (200 mL), dried over anhydrous Na₂SO₄, filtered and concentrated. The residue was purified by silica gel chromatography (0-15% of EtOAc in PE) to give the mixture product 37.4 & 37.4a (3.90 g), which was further purified by SFC (Column: DAICEL CHIRALPAK AD (250 mm*50 mm,*10 um); Condition: CO₂, 0.1% NH₃H₂O EtOH)-ACN; Begin B: 25; End B: 25) to give the product 37.4a (1.0 g, 18.5%) as an off-white solid and the product 37.4 (2.2 g, 40.7%).

37.4a: ¹H NMR (400 MHz, CDCl₃) δ 2.67-2.56 (m, 1H), 2.53-2.38 (m, 3H), 2.34-2.15 (m, 2H), 2.11-2.04 (m, 2H), 1.95-1.64 (m, 6H), 1.63-1.29 (m, 10H), 1.23-1.17 (m, 1H), 1.09 (s, 3H), 1.01 (s, 3H), 0.99-0.87 (m, 1H).

37.4: ¹H NMR (400 MHz, CDCl₃) δ 3.03 (t, J=12.8 Hz, 11H), 2.69-2.56 (m, 1H), 2.49-2.32 (m, 2H), 2.26-2.16 (m, 1H), 2.09-2.03 (m, 1H), 1.95-1.65 (m, 8H), 1.60-1.43 (m, 6H), 1.39-1.21 (m, 5H), 1.09 (s, 3H), 0.97 (s, 3H), 0.91-0.77 (m, 1H).

### Synthesis of 37.5

A fresh prepared MAD (9.45 mmol) solution in toluene (100 mL) was cooled to −70° C., then added to 37.4 (1.00 g, 3.15 mmol) in DCM (10 mL) dropwise at −70° C. After stirring at −70° C. for 1 h, EtMgBr (3.15 mL, 9.45 mmol) was added dropwise and the resulting solution was stirred at −70° C. for another 4 h. The reaction mixture was poured into saturated aqueous citric acid (200 mL) and extracted with EtOAc (2×50 mL). The combined organic layer was dried over Na₂SO₄, filtered and concentrated. The residue was purified by flash column (0-30% of EtOAc in PE) to give product 37.5 (500 mg, 46%). ¹H NMR (400 MHz, CDCl₃)^{6H} 2.67-2.56 (m, 1H), 2.23-2.16 (m, 1H), 2.09-2.01 (m, 1H), 1.88-1.58 (m, 9H), 1.54-1.26 (m, 9H), 1.25-1.09 (m, 6H), 1.08 (s, 3H), 1.04-0.96 (m, 1H), 0.94-0.77 (m, 7H). LC-ELSD/MS: purity>99%, MS ESI calcd. for C₂₃H₃₇O [M−H₂O+H]⁺ 329.3, found 329.3.

### Synthesis of 37.6

To a suspension of Ph₃PEtBr (3.82 g, 10.3 mmol) in anhydrous THF (50 mL) was added t-BuOK (1.15 g, 10.3 mmol) and the mixture was stirred at 60° C. for 30 min. A solution of 37.5 (600 mg, 1.73 mmol) in anhydrous THF (10 mL) was added dropwise. After stirring at 60° C. for 16 h. The mixture was poured into saturated NH₄Cl (100 mL) and stirred for 10 min. The suspension was extracted with EtOAc (2×50 mL) and the combine organic phase was washed with saturated brine (2×100 mL), filtered and concentrated. The residue was purified by flash column (0-25% of EtOAc in PE) to give product 37.6 (600 mg, crude).

### Synthesis of 37.7

To a solution of 37.6 (500 mg, 1.39 mmol) in THE (10 mL) was added BH₃-Me₂S (0.417 mL, 10 M, 4.17 mmol) and the mixture was stirred at 45° C. for 1 h. After cooling 15° C., ethanol (965 mg, 20.8 mmol) was added, followed by NaOH aqueous solution (4.16 mL, 5.0 M, 20.8 mmol). H₂O₂(2.08 mL, 10 M, 20.8 mmol) was added dropwise at 15° C. and the reaction mixture was then heated to 78° C. for 1 hour. After cooling, the mixture was poured into water (100 mL) and extracted with EtOAc (2×50 mL). The organic layer was washed with saturated brine (2×200 mL), drive over anhydrous Na₂SO₄, filtered and concentrated to give 37.7 (500 mg, crude). ¹H NMR (400 MHz, CDCl₃) δ_{H} 1.95-1.89 (m, 1H), 1.85-1.62 (m, 9H), 1.51-1.28 (m, 13H), 1.17-1.02 (m, 8H), 0.88-0.74 (m, 13H).

### Synthesis of 37.8

To a solution of 37.7 (500 mg, 1.32 mmol) in DCM (10 ml) was added PCC (709 mg, 3.30 mmol) and silica gel (800 mg). The mixture was stirred at 25° C. for 1 h then concentrated. The residue was purified by flash column (0-30% of EtOAc in PE) to give product 37.8 (200 mg, 40.4%).

### Synthesis of 37

A solution of 37.8 (50.0 mg, 0.133 mmol) and MeONa (71.8 mg, 1.33 mmol) in MeOH (10 mL) was stirred at 70° C. for 2 days. The reaction mixture was poured into water and extracted was EtOAc (2×20 ml). The combined organic layer was washed with saturated brine (2×50 ml) drive over with anhydrous Na₂SO₄, filtered and concentrated. The residue was purified by flash column (0-30% of EtOAc in PE) to give product 37 (13.1 mg, 26.3%). ¹H NMR (400 MHz, CDCl₃) δ_{H} 2.29 (dd, J=3.2, 12.8 Hz, 1H), 2.14 (s, 3H), 1.91-1.58 (m, 11H), 1.53-1.33 (m, 8H), 1.32-1.15 (m, J=13.1 Hz, 9H), 0.92-0.83 (m, 10H). LC-ELSD/MS: purity>99%, MS ESI calcd. for C₂₅H₄₁O [M−H₂O+H]⁺357.3, found 357.3.

### Synthesis of 38.2

At 0° C., to a solution of 37.8 (200 mg, 0.53 mmol) and HBr (21.1 mg, 0.106 mmol, 40%) in MeOH (10 mL) was added Br₂ (93.7 mg, 0.586 mmol). The mixture was stirred at RT for 2 h then poured into saturated NaHCO₃ (50 mL). The suspension was extracted with EtOAc (3×20 mL) and the combined organic layer was washed with saturated brine (50 mL), dried over anhydrous Na₂SO₄, filtered and concentrated to give 38.2 (150 mg, crude). ¹H NMR (400 MHz, CDCl3) δ_{H} 4.01-3.86 (m, 2H), 2.74-2.48 (m, 2H), 1.98-1.62 (m, 511), 1.49-1.30 (m, 11H), 1.29-1.05 (m, 11H), 0.95-0.85 (m, 10H).

### Synthesis of 38 & 39

To a solution of 38.2 (150 mg, 0.33 mmol) in acetone (5 ml) was added K₂CO₃ (91.0 mg, 0.66 mmol) and 1H-pyrazole-4-carbonitrile (36.8 mg, 0.396 mmol) and the mixture was stirred at RT for 2 h. The reaction mixture was poured into water and extracted with EtOAc (2×20 ml). The combined extract was dried over anhydrous Na₂SO₄, filtered and concentrated. The residue was purified by flash column (0-30% of EtOAc in PE) to give product 39 (20.0 mg, impure) and 38 (9.50 mg, 6.2%). The impure product 39 (20.0 mg) was further purified by HPLC (Column: YMC Triart C18 150*25 mm*5 um); condition: water (10 mM NH₄HCO₃)-ACN; Begin B: 75%; End B: 100%) to afford 39 (5.50 mg, 3.6%).

38: ¹H NMR (400 MHz, CDCl3) δ_{H} 7.82 (s, 1H), 7.81 (s, 1H), 5.11-4.86 (m, 2H), 2.35-2.28 (m, 1H), 1.91-1.66 (m, 9H), 1.48-1.27 (m, 10H), 1.27-1.01 (m, 12H), 0.95 (s, 3H). LC-ELSD/MS: purity>99%, MS ESI calcd. for C₂₉H₄₂N₃O [M−H₂O+H]⁺ 448.3 found 448.3.

39: ¹H NMR (400 MHz, CDCl₃) δ_{H} 7.86 (s, 1H), 7.84 (s, 1H), 5.07-4.85 (m, 2H), 2.51-2.38 (m, 1H), 1.92-1.78 (m, 3H), 1.77-1.56 (m, 9H), 1.53-1.23 (m, 12H), 1.21-1.04 (m, 5H), 0.98 (s, 3H), 0.90-0.85 (m, 6H). LC-ELSD/MS: purity>95%, MS ESI calcd. for C₂₉H₄₂N₃O [M−H₂O+H]⁺ 448.3 found 448.3.

### Example 40: Synthesis of 1-(2-((1S,4aS,4bR,6aS,8R,11aS,11bS,13aS)-8-hydroxy-8,11a,13a-trimethyloctadecahydro-1H-cyclohepta[a]phenanthren-1-yl)-2-oxoethyl)-1H-pyrrole-3-carbonitrile (40)

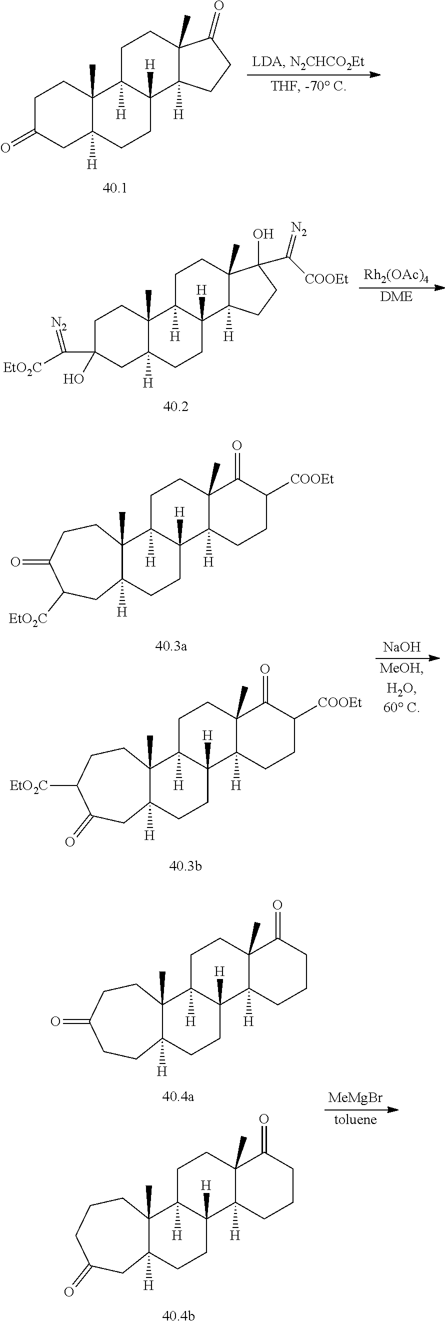

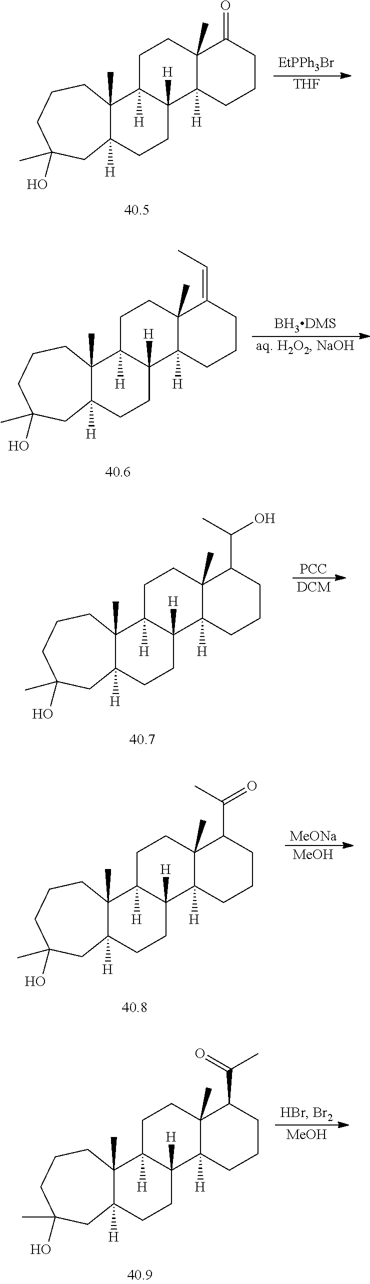

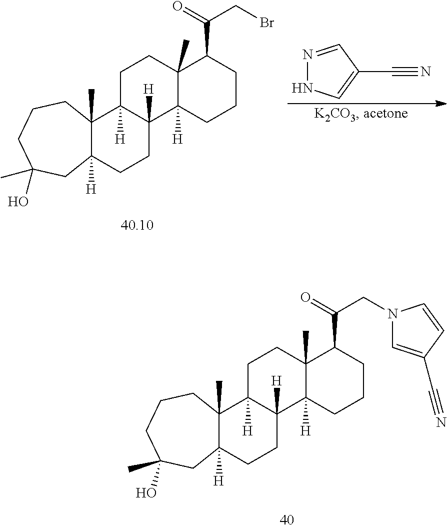

### Synthesis of 40.2

An LDA solution (346 mmol) was cooled to −70° C. and added to a stirred solution of 40.1 (20.0 g, 69.3 mmol) and ethyl diazoacetate (39.4 g, 346 mmol) in THF (500 mL). After stirring at −70° C. for 3 h, HOAc (20.7 g, 346 mmol) in THF (50 mL) was added and the mixture was warmed to 20° C. for 16 h. Water (800 mL) and PE (200 mL) was added, and the layers were separated. The aqueous phase was extracted with EtOAc (300 mL) and the combined organic layers were washed with saturated brine (1000 mL), dried over anhydrous Na₂SO₄, filtered and concentrated to give the product 40.2 (52.0 g, crude), which was used directly to the next step.

### Synthesis of 40.3a & 40.3b

To a solution of 40.2 (52.0 g, 100 mmol) in DME (400 mL) was added Rh₂ (OAc)₄ (883 mg, 2 mmol) and the reaction mixture was stirred at RT for 16 h. The reaction mixture was concentrated, and the residue was purified by silica gel chromatography (0, —20% of EtOAc in PE) to give the impure mixture of products 40.3a & 40.3b (36.5 g, 79.2%), which was used directly in next step.

### Synthesis of 40.4a & 44.4b

To a solution of 40.3a & 40.3b (36.5 g, 79.2 mmol) in MeOH (200 mL) was added H₂O (20 mL) and NaOH (25.3 g, 633 mmol). The reaction mixture was stirred at 60° C. for 16 h to give a yellow mixture. The reaction mixture was concentrated, then H₂O (500 mL) was added. The mixture was extracted with EtOAc (2×500 mL) and the combined organic phase was washed with HCl (500 mL, 1M) saturated brine (500 mL), dried over anhydrous Na₂SO₄, filtered and concentrated. The residue was purified by flash column (0˜30% of EtOAc in PE), triturated from EtOAc/PE(10/1, 165 mL) and purified by SFC (Column: DAICEL CHIRALPAK AD(250 mm*50 mm*10 μm); Condition: 0.1% NH₃H₂O EtOH; Begin B: 35%; End B: 35%; Flow Rate (ml/min): 200; Injections: 300) to get 40.4a (6.50 g, 26.4%) and 40.4b (6.00 g, 24.4%)

40.4a: ¹H NMR (400 MHz, CDCl₃) δ_{H} 2.65-2.42 (m, 4H), 2.36-2.27 (m, 1H), 2.23-2.16 (m, 1H), 2.08-1.97 (m, 2H), 1.90-1.67 (m, 5H), 1.51-1.15 (m, 11H), 1.08 (s, 3H), 0.89-0.83 (m, 1H), 0.80-0.70 (m, 4H). LC-ELSD/MS: purity 99%; MS ESI calcd. for C₂₁H₃₃O₂[M+H]⁺ 317.3, found 317.3, MS ESI calcd. for C₂₁H₃₁O [M+H-H₂O]299.2, found 299.2.

40.4b: ¹H NMR (400 MHz, CDCl3) δ_{H} 2.85-2.75 (m, 1H), 2.68-2.56 (m, 1H), 2.51-2.31 (m, 2H), 2.24-2.16 (m, 1H), 2.10-1.57 (m, 10H), 1.53-1.11 (m, 9H), 1.08 (s, 3H), 1.07-0.90 (m, 1H), 0.87 (s, 3H), 0.81-0.73 (m, 1H). LC-ELSD/MS: purity 99%; MS ESI calcd. for C₂₁H₃₃O₂[M+H]⁺ 317.3, found 317.3, MS ESI calcd. for C₂₁H₃₁O [M+H-H₂O]⁺299.2, found 299.2.

### Synthesis of 40.5

At 0° C., to a solution of 40.4b (4.00 g, 12.6 mmol) in THF (40 mL) was added MeMgBr (5.86 mL, 17.6 mmol, 3M in ethyl ether). After stirring at 20° C. for 16 h, the mixture was poured into saturated NH₄Cl (200 mL) and extracted with EtOAc (2×200 mL). The combined organic layer was dried over Na₂SO₄, filtered and concentrated. The residue was purified by silica gel chromatography (0-25% EtOAc in PE) to give 40.5 (1.70 g, impure). ¹H NMR (400 MHz, CDCl₃) δ_{H} 2.68-2.56 (m, 1H), 2.23-2.14 (m, 1H), 2.08-1.65 (m, 9H), 1.55-1.23 (m, 11H), 1.18 (s, 3H), 1.07 (s, 3H), 1.04-0.79 (m, 5H), 0.75 (s, 3H).

### Synthesis of 40.6

To a solution of PPh₃EtBr (13.3 g, 36 mmol) in THF (100 mL) was added t-BuOK (4.03 g, 36 mmol) and the reaction mixture was stirred at 60° C. for 0.5 h. A solution of 40.5 (2.00 g, 6.01 mmol) in THE (20 mL) was added and the reaction mixture was stirred at 60° C. for another 12 h. The mixture was poured into saturated MH₄Cl (100 mL) and extracted with EtOAc (2×100 mL). The combined organic phase was washed with saturated brine (2×100 mL), dried over anhydrous Na₂SO₄, filtered and concentrated. The residue was purified by silica gel chromatography (0-5% EtOAc in PE) to get 40.6 (1.50 g, 72.4%). ¹H NMR (400 MHz, CDCl₃)⁶H 5.21-5.07 (m, 1H), 2.21-1.58 (m, 13H), 1.50-1.24 (m, 7H), 1.23-1.03 (m, 10H), 0.93-0.66 (m, 9H).

### Synthesis of 40.7

To a solution of 40.6 (1.50 g, 4.35 mmol) in THF (30 mL) was added BH₃-Me₂S (1.73 mL, 10 M, 17.30 mmol) and the mixture was stirred at RT for 16 h. The mixture was cooled to 15° C. and ethanol (2.55 mL) was added, followed by NaOH aqueous (8.68 mL, 5.0 M, 43.4 mmol). Hydrogen peroxide (4.34 mL, 10 M, 43.4 mmol) was added dropwise at 0° C. and the reaction mixture was stirred at 70° C. for 1 hour. After cooling 15° C., Na₂S₂O₃ (100 mL, sat. aq.) was added and the suspension was extracted with EtOAc (2×100 mL). The combined organic layer was washed with brine (2×50 mL), dried over Na₂SO₄, filtered and concentrated to give 40.7 (1.80 g, crude), which was used directly to the next step. ¹H NMR (400 MHz, CDCl₃)⁶H 4.33-4.03 (m, 1H), 1.95-1.48 (m, 12H), 1.37-1.20 (m, 10H), 1.18 (s, 3H), 1.17-0.81 (m, 10H), 0.80-0.69 (m, 6H).

### Synthesis of 40.8

To a solution of 40.7 (1.70 g, 4.68 mmol) in DCM (20 mL) was added PCC (2.01 g, 9.36 mmol) and silica gel (2.01 g). After stirring at 25° C. for 0.5 h, the mixture was filtered, and filter cake was washed with EtOAc (2×50 mL). The filtrate was concentrated, and the residue was purified by silica gel chromatography (0-20% EtOAc in PE) to get 40.8 (1.10 g, 65.1%). ¹H NMR (400 MHz, CDCl₃)⁶H 2.45 (d, J=5.2 Hz, 0.5H), 2.30-2.23 (m, 0.5H), 2.15-2.08 (m, 3H), 1.90-1.67 (m, 6H), 1.58-1.20 (m, 16H), 1.17 (d, J=4.8 Hz, 3H), 1.13-0.92 (m, 3H), 0.91-0.88 (m, 3H), 0.87-0.74 (m, 3H), 0.72 (d, J=1.8 Hz, 2H).

### Synthesis of 40.9

To a solution of 40.8 (900 mg, 2.49 mmol) in MeOH (50 mL) was added MeONa (2.69 g, 49.8 mmol) and the reaction was stirred at 80° C. for 48 h. The residue was poured into saturated NH₄Cl (100 mL), then extracted with EtOAc (2×100 mL). The combined organic phase was washed with saturated brine (100 mL), dried over anhydrous Na₂SO₄, filtered and concentrated. The residue was purified by silica gel chromatography (15-20% EtOAc in PE) to give 40.9 (900 mg, 100%). ¹H NMR (400 MHz, CDCl₃)⁶H 2.31-2.22 (m, 1H), 2.15-2.11 (m, 3H), 1.95-1.57 (m, 11H), 1.50-1.16 (m, 13H), 0.98-0.70 (m, 12H).

### Synthesis of 40.10

To a solution of 40.9 (200 mg, 0.55 mmol) in MeOH (10 ml) was added HBr (22.4 mg, 0.11 mmol, 40% in water) and Br₂ (106 mg, 0.66 mmol). After stirring at 20° C. for 1 h, the reaction was quenched with saturated aqueous NaHCO₃ (10 mL) and extracted with EtOAc (2×30 mL). The combined organic phase was washed with brine (30 mL), dried over anhydrous Na₂SO₄, filtered and concentrated to afford 40.10 (250 mg, crude), which used directly for the next step. ¹H NMR (400 MHz, CDCl₃) δ_{H} 4.01-3.87 (m, 2H), 2.70-2.51 (m, 1H), 1.88-1.68 (m, 7H), 1.51-1.21 (m, 13H), 1.18 (s, 3H), 1.03-0.94 (m, 3H), 0.93-0.91 (m, 3H), 0.88-0.76 (m, 4H), 0.72 (s, 3H).

### Synthesis of 40

To a solution of 40.10 (250 mg, 0.57 mmol) in acetone (10 mL) was added 4-cyanopyrazole (79.4 mg, 0.85 mmol) and K₂CO₃ (79.7 mg, 0.57 mmol). After the mixture was stirred at RT for 16 h, water (50 mL) was added and the mixture was extracted with EtOAc (3×50 mL). The combined organic layer was concentrated, and the residue was purified by flash column (0˜30% of EtOAc in PE) to give 40 (112.1 mg, 43.7%).

40: ¹H NMR (400 MHz, CDCl₃)⁶H 7.81 (d, J=6.0 Hz, 2H), 5.07-4.91 (m, 2H), 2.35-2.27 (m, 1H), 1.90-1.59 (m, 10H), 1.52-1.17 (m, 14H), 1.02-0.71 (m, 12H). LC-ELSD/MS: purity 99%; MS ESI calcd. for C₂₅H₄₀N₃O [M+H-H₂O]⁺ 434.3, found 434.3.

### Example 41: Synthesis of 1-(3-((1S,4aS,4bR,6aS,8R,11aS,11bS,13aS)-8-hydroxy-8,11a,13a-trimethyloctadecahydro-1H-cyclohepta[a]phenanthren-1-yl)-3-oxopropyl)-1H-pyrazole-4-carbonitrile (41)

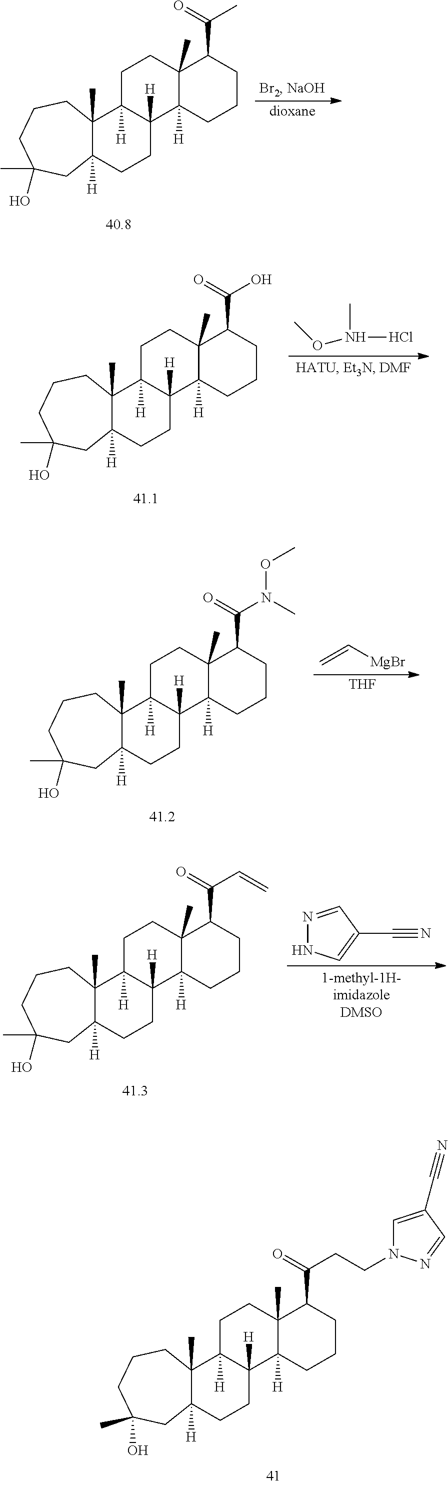

### Synthesis of 41.1

Br₂ (1.55 g, 9.7 mmol) was added to a solution of NaOH (2.32 g, 11.6 mL, 58.1 mmol, 5 M). The mixture was stirred at 25° C. for 20 min and added to a solution of 40.8 (700 mg, 1.94 mmol) in dioxane (20 mL) and water (5 mL). After stirring at RT for 16 h, the resulting mixture was poured into aqueous Na₂SO₃ (30 mL), then stirred at 70° C. for 1 hour. After cooling, the mixture was acidified with HCl (1 M in water, 50 mL) and stirred at RT for 10 min. The precipitated solid was filtered out, washed with water (2×50 mL) and dried to give 41.1 (630 mg, 89.6%). ¹H NMR (400 MHz, DMSO) δ_{H} 4.05-3.92 (m, 1H), 1.96-1.88 (m, 1H), 1.72-1.42 (m, 12H), 1.33-1.10 (m, 9H), 1.03 (s, 3H), 0.92-0.86 (m, 2H), 0.83 (s, 3H), 0.80-0.70 (m, 4H), 0.68 (s, 3H).

### Synthesis of 41.2

To a solution of 41.1 (630 mg, 1.73 mmol) in DMF (20 mL) was added N,O-dimethylhydroxylamine hydrochloride (674 mg, 6.92 mmol), HATU (1.31 g, 3.46 mmol) and TEA (2.39 mL, 17.3 mmol). After the reaction mixture was stirred at RT for 16 hours, water (30 mL) was added. The mixture was extracted with ethyl acetate (2×50 mL) and the combined organic phase was washed with water (2×50 mL), dried over Na₂SO₄, filtered, concentrated. The residue was purified by flash column (0-20% of EtOAc in PE) to give 41.2 (370 mg, 52.7%). ¹H NMR (400 MHz, CDCl₃) δ_{H} 3.72-3.62 (m, 3H), 3.22-3.13 (m, 3H), 1.96-1.63 (m, 8H), 1.55-1.21 (m, 14H), 1.18 (s, 3H), 0.99 (s, 3H), 0.97-0.77 (m, 6H), 0.73 (s, 3H).

### Synthesis of 41.3

To a solution of 41.2 (270 mg, 0.66 mmol) in THF (10 mL) was added vinylmagnesium bromide (9.5 mL, 0.7 M, 6.65 mmol). After stirring at RT for 12 h, 10% NH₄Cl (50 ml) was added and the suspension was extracted with EA (2×30 mL). The combined organic phase was washed with saturated NaCl (2×50 mL), dried over Na₂SO₄, filtered, concentrated. The residue was purified by combi-flash (0-15% of EtOAc in PE) to give 41.3 (100 mg, impure), which was used directly to the next step.

### Synthesis of 41 & 41a

A solution of 41.3 (100 mg, 0.27 mmol), 1-methyl-1H-imidazole (65.1 mg, 0.80 mmol) and 1H-pyrazole-4-carbonitrile (49.9 mg, 0.54 mmol) in DMSO (5 mL) was stirred at 70° C. for 16 h. The mixture was poured into saturated brine (50 mL), then extracted with EtOAc (3×20 mL). The combined organic layer was washed with saturated brine (2×50 mL), dried over anhydrous Na₂SO₄, filtered and concentrated. The residue was purified by pre-HPLC (Column: Welch Xtimate C18 150*25 mm*5 μm; Condition: water (0.225% TFA)-ACN; Begin B: 58; End B: 88; Gradient Time (min): 8.5; 100% B Hold Time (min): 2; Flow Rate (ml/min):30; Injections: 7) to get 41 (2.40 mg, 1.93%).

41: ¹H NMR (400 MHz, CDCl₃) δ_{H} 7.89 (s, 1H), 7.75 (s, 1H), 4.43-4.31 (m, 2H), 3.07-2.94 (m, 2H), 2.26-2.14 (m, 1H), 1.90-1.69 (m, 6H), 1.37-1.21 (m, 7H), 1.20-0.88 (m, 15H), 0.82 (s, 3H), 0.80-0.74 (m, 2H), 0.71 (s, 3H). LC-ELSD/MS: purity 99%; MS ESI calcd. for C₂₉H₄₄N₃O₂ [M+H]⁺ 466.3, found 466.3; MS ESI calcd. for C₂₉H₄₂N₃O [M−H₂O+H]⁻ 448.3, found 448.3.

### Example 42: Synthesis of 1-(2-((1S,4aS,4bR,6aS,8R,11aS,11bS,13aS)-8-ethyl-8-hydroxy-11a,13a-dimethyloctadecahydro-1H-cyclohepta[a]phenanthren-1-yl)-2-oxoethyl)-1H-pyrazole-4-carbonitrile (42)

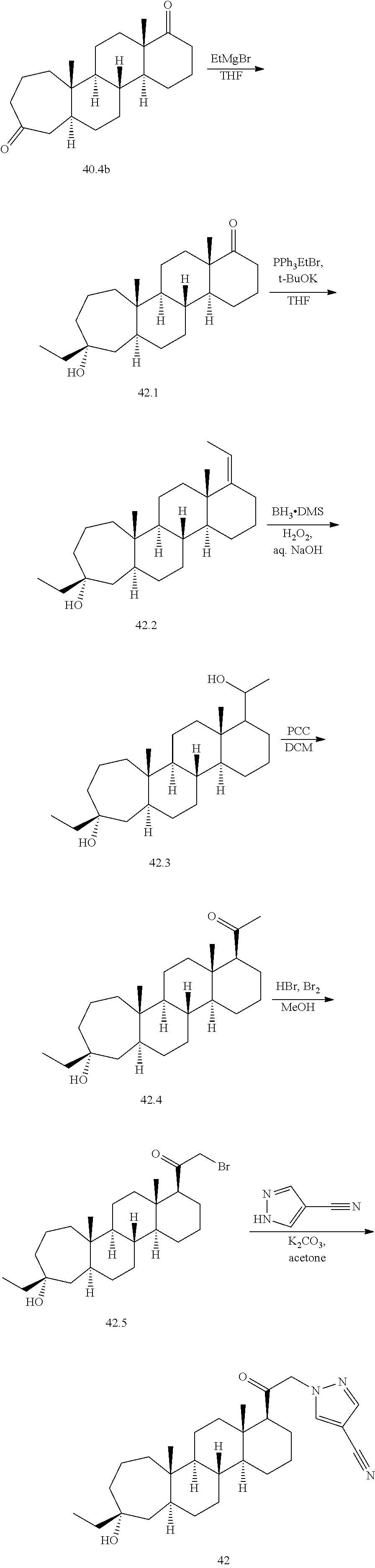

### Synthesis of 42.1

At 0° C., to a mixture of 40.4b (2.50 g, 7.89 mmol) in THF (20 mL) was added EtMgBr (3.93 ml, 11.8 mmol, 3 M) and the mixture was stirred at 25° C. for 16 h. NH₄Cl (50 mL) was added and the suspension was extracted with EtOAc (3×50 mL). The combined organic phase was washed with saturated brine (2×50 mL), dried over anhydrous Na₂SO₄, filtered and concentrated. The residue was purified by flash column (0-15% of EtOAc in PE) to give 42.1 (730 mg, 26.5%). ¹H NMR (400 MHz, CDCl₃)⁶H 2.67-2.56 (m, 1H), 2.22-2.14 (m, 1H), 2.09-1.99 (m, 1H), 1.90-1.60 (m, 8H), 1.50-1.09 (m, 15H), 1.07 (s, 3H), 1.00-0.79 (m, 6H), 0.75 (s, 3H). LC-ELSD/MS 30-90AB-2 min-E, purity>99%, MS ESI calcd. for C₂₃H₃₉O₂ [M−H₂O+H]⁺ 329.3, found 392.3.

### Synthesis of 42.2

To a mixture of EtPPh₃Br (4.71 g, 12.7 mmol) in THE (17 ml) was added t-BuOK (1.42 g, 12.7 mmol) at 15° C. The resulting mixture was then stirred at 50° C. for 45 mins. 42.1 (740 mg, 2.13 mol) in THE (2 ml) was added in portions below 50° C. and the reaction mixture was stirred at 50° C. for another 12 h. The reaction was quenched with 10% NH₄Cl aqueous (30 ml) and the layers were separated. The aqueous was extracted with EtOAc (50 ml) and the combined organic phase was washed with saturated brine (2×50 mL), dried over anhydrous Na₂SO₄, filtered and concentrated. The residue was purified by flash column (0-15% of EtOAc in PE) to give 42.2 (390 mg, 51%). ¹H NMR (400 MHz, CDCl₃) δ_{H} 5.23-5.03 (m, 1H), 2.56-2.41 (m, 1H), 2.26-1.97 (m, 1H), 1.96-1.69 (m, 7H), 1.66-1.57 (m, 5H), 1.54-1.02 (m, 16H), 0.97-0.87 (m, 6H), 0.80-0.69 (m, 5H).

### Synthesis of 42.3

To a solution of 42.2 (390 mg, 1.08 mmol) in THF (5 mL) was added BH₃-Me₂S (540 μL, 10 M, 5.40 mmol) and the mixture was stirred at RT for 20 h. After cooling the mixture to 0° C., ethanol (943 μL, 16.2 mmol) was added, followed by aqueous NaOH (3.23 mL, 5.0 M, 16.2 mmol). Hydrogen peroxide (1.61 mL, 10 M, 16.2 mmol) was added dropwise and the reaction mixture was then stirred at 70° C. for 1 h. The mixture was cooled to 15° C., Na₂S₂O₃ (20 mL, sat. aq.) was added. The resultant suspension was extracted with EtOAc (2×20 mL) and the combined organic phase was washed with saturated brine (2×50 mL), dried over anhydrous Na₂SO₄, filtered and concentrated to give 42.3 (360 mg, impure).

¹H NMR (400 MHz, CDCl₃) δ_{H} 4.36-3.99 (m, 1H), 1.94-1.61 (m, 9H), 1.55-1.37 (m, 6H), 1.37-1.01 (m, 14H), 1.00-0.83 (m, 8H), 0.82-0.65 (m, 6H).

### Synthesis of 42.4

To a solution of 42.3 (360 mg, 0.96 mmol) in DCM (4 mL) at 0° C. was added silica gel (410 mg) and PCC (408 mg, 1.90 mmol) and the mixture was stirred at RT for 2h. PE (30 mL) was added and the resulting mixture was filtered through a pad of silica gel and the filter cake was washed with DCM (3×30 mL). The filtrate was concentrated, and the residue was purified by flash column (0-15% of EtOAc in PE) to give 42.4 (70.0 mg, 19.6%).

¹H NMR (400 MHz, CDCl3) δ_{H} 2.33-2.21 (m, 1H), 2.13 (s, 3H), 1.92-1.63 (m, 8H), 1.50-1.40 (m, 5H), 1.36-1.14 (m, 10H), 0.94-0.86 (m, 9H), 0.83-0.74 (m, 3H), 0.72 (s, 3H).

### Synthesis of 42.5

To a solution of 42.4 (70.0 mg, 186 μmol) in MeOH (1 mL) was added HBr (7.42 mg, 37.2 μmol, 40%) and Br₂ (32.5 mg, 204 μmol) and the mixture was stirred at RT for 4h. To the reaction mixture was added NaHCO₃ (10 ml, sat. aq.) and the suspension was extracted with EtOAc (2×10 mL). The combined organic phase was washed with saturated brine (2×30 mL), dried over anhydrous Na₂SO₄, filtered, and concentrated. The residue was purified by flash column (0˜20% of EtOAc in PE) to give 42.5 (46.0 mg, 54.5%). ¹H NMR (400 MHz, CDCl3) δ_{H} 3.96-3.87 (m, 2H), 3.15-3.02 (m, 1H), 2.62-2.48 (m, 1H), 2.04 (s, 1H), 1.89-1.59 (m, 11H), 1.38-1.13 (m, 13H), 0.96-0.88 (m, 6H), 0.83-0.75 (m, 4H), 0.73-0.71 (m, 2H).

### Synthesis of 42

To a solution of 42.5 (46.0 mg, 101 μmmol) in acetone (5 mL) was added 1H-pyrazole-4-carbonitrile (14.0 mg, 151 mol) and K₂CO₃ (41.8 mg, 303 μmol) and the mixture was stirred at RT for 16 h. The mixture was treated with water (20 mL) and extracted with EtOAc (2×20 mL). The combined organic phase was washed with brine (20 mL), dried over anhydrous Na₂SO₄, filtered and concentrated. The residue was purified by flash column (0˜30% of EtOAc in PE) to give 42 (2.0 mg, 4.25%). ¹H NMR (400 MHz, CDCl3)⁶H 7.82 (s, 1H), 7.81 (s, 1H), 5.08-4.91 (m, 2H), 2.31 (dd, J=12.8, 3.6 Hz, 1H), 1.90-1.70 (m, 6H), 1.68-1.59 (m, 4H), 1.47-1.39 (m, 3H), 1.33-1.20 (m, 10H), 1.08 (s, 1H), 0.97 (s, 1H), 0.93 (s, 3H), 0.92-0.87 (m, 4H), 0.84-0.76 (m, 3H), 0.73 (s, 3H). LC-ELSD/MS purity>99%, MS ESI calcd. for C₂₉H₄₃N₃O₂ [M−H₂O+H]⁺ 448.3, found 448.3.

### Example 43: Synthesis of 1-(2-((1S,4aS,4bR,6aS,8R,11aS,11bS,13aS)-8-hydroxy-8-(methoxymethyl)-11a,13a-dimethyloctadecahydro-1H-cyclohepta[alphenanthren-1-yl)-2-oxoethyl)-1H-pyrazole-4-carbonitrile (43)

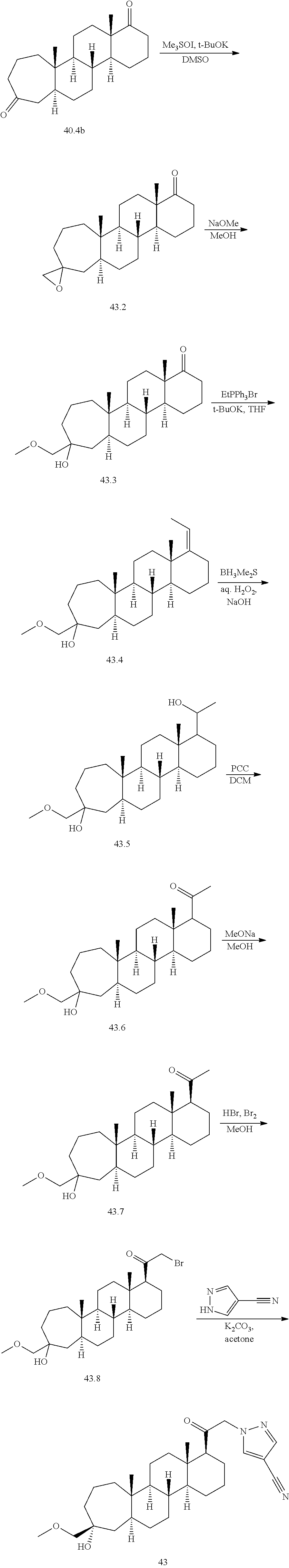

### Synthesis of 43.2

To a suspension of Me₃SOI (831 mg, 3.8 mmol) in DMSO (10 mL) was added t-BuOK (423 mg, 3.8 mmol) and the reaction mixture was stirred at 60° C. for 1 h. After cooling to RT, 40.4b (1.00 g, 3.2 mmol) was added and the mixture was stirred at RT for 2 h. The reaction mixture was poured into water (50 mL) while stirring and the resultant suspension was filtered. The filter cake was washed with water (2×20 mL) and dried to give 43.2 (1.60 g, crude). ¹H NMR (400 MHz, CDCl₃)^{6H} 2.75-2.85 (m, 1H) 2.57-2.67 (m, 2H) 2.31-2.49 (m, 2H) 2.06-2.22 (m, 2H) 1.61-1.98 (m, 9H) 1.32-1.49 (m, 6H) 1.12-1.22 (m, 2H) 1.07-1.09 (m, 3H) 0.89-1.06 (m, 2H) 0.87 (s, 3H) 0.73-0.81 (m, 2H).

### Synthesis of 43.3

Na (763 mg, 33.2 mmol) was added into MeOH (20 mL) in several small portions and the mixture was stirred at RT for 2. A solution of 43.2 (1.10 g, 3.32 mmol) in THF (10 mL) was added and the mixture was warmed and stirred at 60° C. for 16 h. After cooling, the mixture was poured into saturated NH₄Cl (50 mL) and the suspension was extracted with EtOAc (3×50 mL). The combined organic layer was washed with saturated brine (100 mL), dried over anhydrous Na₂SO₄, filtered and concentrated. The residue was purified by flash column (0-30% of EtOAc in PE) to give 43.3 (790 mg, impure). ¹H NMR (400 MHz, CDCl3) δ_{H} 3.36-3.38 (m, 3H) 3.13-3.20 (m, 2H) 2.55-2.67 (m, 1H) 2.12-2.22 (m, 2H) 1.64-1.87 (m, 10H) 1.30-1.42 (m, 5H) 1.10-1.22 (m, 4H) 1.07 (s, 3H) 0.78-0.97 (m, 5H) 0.71-0.75 (m, 3H).

### Synthesis of 43.4

To a mixture of EtPPh₃Br (12.1 g, 32.7 mmol) in THE (25 mL) was added t-BuOK (3.66 g, 32.7 mmol) and the resulting mixture was stirred at 60° C. for 30 min. 43.3 (1.98 g, 5.5 mmol) was added in portions and the reaction mixture was stirred at 60° C. for 16 h. The yellow suspension was cooled and quenched with 10% NH₄Cl aqueous (100 mL) solution. The layers were separated, and the aqueous layer was extracted with EtOAc (2×50 mL). The combined organic phase was dried over anhydrous Na₂SO₄, filtered and concentrated. The residue was purified by trituration with MeOH/H₂O (1:1, 50 mL) at reflux to give 43.4 (2.50 g, crude), which was further purified by flash column (0˜30% of EtOAc in PE) to give 43.4 (700 mg, 28%). ¹H NMR (400 MHz, CDCl₃) δ_{H} 5.10-5.21 (m, 1H) 3.35-3.43 (m, 3H) 3.09-3.26 (m, 2H) 2.49 (m, 1H) 2.12-2.17 (m, 1H) 1.56-2.01 (m, 14H) 1.29-1.54 (m, 7H) 1.04-1.22 (m, 5H) 1.03 (s, 1H) 0.90 (s, 2H) 0.77-0.84 (m, 2H) 0.72-0.76 (m, 3H).

### Synthesis of 43.5

To a solution of 43.4 (700 mg, 1.9 mmol) in THE (10 mL) was added BH₃-Me₂S (1.86 mL, 1OM, 18.6 mmol) and the mixture was stirred at RT for 16 h. The reaction was cooled to 0° C. and EtOH (5.42 mL, 93.0 mmol) was added, followed by NaOH (18.5 mL, 5.0M, 93.0 mmol). H₂O₂(9.29 mL, 93.0 mmol, 30% in water) was added dropwise while keeping the internal temperature below 15° C. The mixture was then heated to 70° C. and stirred at for 1 h. The mixture was cooled and poured into water (50 mL) and extracted with EtOAc (2×50 mL). The combined organic layer was washed with saturated Na₂S₂O₃ (100 mL), brine (2×100 mL), dried over anhydrous Na₂SO₄, filtered and concentrated to give 43.5 (1.00 g, crude). ¹H NMR (400 MHz, CDCl₃) δ_{H} 3.32-3.44 (m, 3H) 3.11-3.27 (m, 2H) 2.99 (s, 1H) 1.72-1.89 (m, 5H) 1.30-1.49 (m, 7H) 0.94-1.20 (m, 12H) 0.71-0.93 (m, 14H).

### Synthesis of 43.6

At 0° C., to a solution of 43.5 (1.00 g, 2.5 mmol) in DCM (10 mL) was added silica gel (821 mg) and PCC (808 mg, 3.75 mmol). The mixture was stirred at 25° C. for 1 h and PE (5 mL) was added. The resulting mixture was filtered through a pad of silica gel and the filter cake was washed with DCM (2×20 mL). The filtrate was concentrated, and the residue was purified by column chromatography on silica gel (6%-10% of EtOAc in PE) to give 43.6 (220 mg, 22%). ¹H NMR (400 MHz, CDCl₃) δ_{H} 3.36-3.40 (m, 3H) 3.11-3.25 (m, 2H) 2.24-2.47 (m, 1H) 2.11-2.14 (m, 3H) 1.58-1.85 (m, 10H) 1.28-1.57 (m, 8H) 0.91-1.23 (m, 6H) 0.90 (s, 3H) 0.75-0.87 (m, 3H) 0.72 (s, 3H).

### Synthesis of 43.7

At 0° C., to a solution of 43.6 (220 mg, 0.6 mmol) in MeOH (15 mL) was added MeONa (605 mg, 11.2 mmol), and the mixture was stirred at 80° C. for 16 h. After cooling, the mixture was poured into saturated NH₄Cl (100 mL) and the resultant suspension was extracted with EtOAc (2×50 mL). The combined organic phase was washed with saturated brine (100 mL), dried over anhydrous Na₂SO₄, filtered, concentrated. The residue was purified by flash column (0˜20% of EtOAc in PE) to give 43.7 (180 mg, 82%). ¹H NMR (400 MHz, CDCl₃) δ_{H} 3.35-3.41 (m, 3H) 3.10-3.25 (m, 2H) 2.20-2.30 (m, 1H) 2.12-2.16 (m, 3H) 1.59-1.83 (m, 10H) 1.33-1.54 (m, 6H) 0.91-1.32 (m, 11H) 0.90 (s, 3H) 0.72 (s, 3H).

### Synthesis of 43.8

To a solution of 43.7 (180 mg, 0.5 mmol) in MeOH (2 mL) was added HBr (40%, 18.4 mg, 0.1 mmol) and Br₂ (80.9 mg, 0.5 mmol) dropwise and the mixture was stirred at RT for 2 h. NaHCO₃ (50 ml, sat. aq.) was added and the mixture was extracted with EtOAc (2×30 mL). The combined organic phase was washed with saturated brine (2×50 mL), dried over anhydrous Na₂SO₄, filtered, and concentrated. The residue was purified by flash column (0˜30% of EtOAc in PE) to give 43.8 (180 mg, crude). ¹H NMR (400 MHz, CDCl₃)^{6H} 3.87-4.01 (m, 2H) 3.37-3.39 (m, 3H) 3.11-3.23 (m, 2H) 2.50-2.59 (m, 1H) 1.65-1.84 (m, 9H) 1.31-1.53 (m, 8H) 0.94-1.21 (m, 6H) 0.91-0.93 (m, 3H) 0.79 (s, 4H) 0.72 (s, 3H).

### Synthesis of 43

To a solution of 43.8 (180 mg, 0.4 mmol) in acetone (2 ml) was added 4-cyanopyrazole (39.2 mg, 0.4 mmol) and K₂CO₃ (105 mg, 0.8 mmol). The mixture was stirred at RT for 2 h then poured to water (20 ml). The resultant suspension was stirred for 10 min then extracted with EtOAc (2×20 mL). The combined organic phase was washed with saturated brine (2×50 mL), dried over anhydrous Na₂SO₄, filtered and concentrated. The residue was purified by combi-flash (0-40% of EtOAc in PE) to give 43.9 (110 mg), which was further purified by SFC (Method: Column: DAICEL CHIRALCEL OD-H (250 mm*30 mm, 5 um); Condition: CO₂, 0.1% NH₃H₂O EtOH; Begin B: 35%; End B: 35%) to afford 43 (76.0 mg, 70%).

43: ¹H NMR (400 MHz, CDCl₃) δ_{H} 7.82 (s, 1H) 7.81 (s, 1H) 4.90-5.08 (m, 2H) 3.38 (s, 3H) 3.11-3.18 (m, 2H) 2.31 (dd, J=12.8, 3.2 Hz, 1H) 2.17 (s, 1H) 1.60-1.88 (m, 9H) 0.94-1.52 (m, 14H) 0.93 (s, 3H) 0.76-0.86 (m, 3H) 0.72 (s, 3H). LC-ELSD/MS: purity 99%, analytic SFC: 100% de, MS ESI calcd. for C₂₉H₄₃N₃O₃ [M+H]⁺ 482.3, found 482.4.

### Example 44 & 45: Synthesis of 1-(2-((1S,4aS,4bR,6aS,9S,11aS,11bS,13aS)-9-hydroxy-9,11a,13a-trimethyloctadecahydro-1H-cyclohepta[a]phenanthren-1-yl)-2-oxoethyl)-1H-pyrazole-4-carbonitrile (44) & Synthesis of 1-(2-((1R,4aS,4bR,6aS,9S,11aS,11bS,13aS)-9-hydroxy-9,11a,13a-trimethyloctadecahydro-1H-cyclohepta[a]phenanthren-1-yl)-2-oxoethyl)-1H-pyrazole-4-carbonitrile (45)

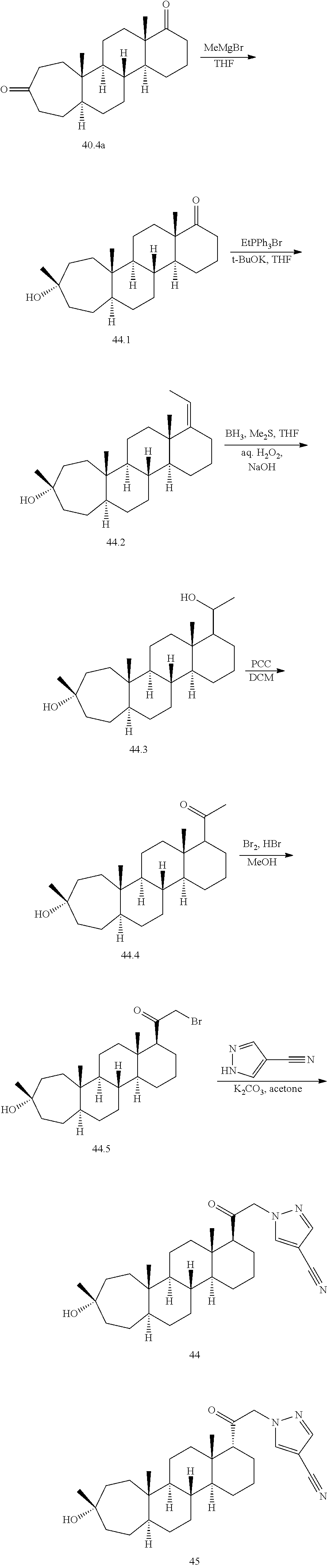

### Synthesis of 44.1

At 0° C., to a solution of 40.4a (4.50 g, 14.2 mmol) in THE (100 mL) was added MeMgBr (6.6 mL, 19.8 mmol, 3M in ethyl ether) and the mixture was stirred at RT for 12 h. The reaction mixture was poured into saturated NH₄Cl (100 mL) and extracted with EtOAc (2×200 mL). The combined organic layer was dried over Na₂SO₄, filtered and concentrated. The residue was purified by silica gel chromatography (0-25% EtOAc in PE) to give 44.1 (1.10 g, 23.3%). 44.1 ¹H NMR (400 MHz, CDCl₃)^{6H} 2.67-2.55 (m, 1H), 2.23-2.15 (m, 1H), 2.09-1.61 (m, 9H), 1.54-1.25 (m, 9H), 1.23-1.10 (m, 7H), 1.07 (s, 3H), 0.92-0.71 (m, 6H).

### Synthesis of 44.2

To a solution of PPh₃EtBr (11.3 g, 30.6 mmol) in THF (100 mL) was added t-BuOK (3.43 g, 30.6 mmol) and the reaction mixture was stirred at 60° C. for 30 min. A solution of 44.1 (1.70 g, 5.11 mmol) in THF (20 mL) was added and the mixture was stirred at 60° C. for 12 h. The mixture was poured into saturated NH₄Cl (100 mL) and extracted with EtOAc (2×100 mL). The combined organic phase was washed with saturated brine (2×100 mL), dried over anhydrous Na₂SO₄, filtered and concentrated. The residue was purified by silica gel chromatography (0-5% EtOAc in PE) to afford 44.2 (1.50 g, impure). ¹H NMR (400 MHz, CDCl₃) δ_{H} 5.20-5.10 (m, 1H), 1.98-1.52 (m, 16H), 1.38-1.23 (m, 7H), 1.20 (s, 3H), 1.18-1.04 (m, 5H), 0.94-0.72 (m, 8H).

### Synthesis of 44.3

To a solution of 44.2 (1.50 g, 4.35 mmol) in THE (30 mL) was added BH₃ Me₂S (1.73 mL, 17.4 mmol, 10 M) and the mixture was stirred at RT for 16 h. The resulting mixture was cooled to 0° C. and ethanol (2.66 mL) was added, followed by aqueous NaOH solution (8.68 mL, 5.0 M, 43.4 mmol). Hydrogen peroxide (4.34 mL, 10 M, 43.4 mmol) was added dropwise while keeping the internal temperature below 15° C. The reaction mixture was warmed to 70° C. and stirred for 1 ho. After cooling, Na₂S₂O₃ (100 mL, sat. aq.) was added and the mixture was extracted with EtOAc (100 mL×3). The combined organic layer was washed with brine (2×50 mL), dried over Na₂SO₄, filtered and concentrated to give 44.3 (1.70 g, crude). ¹H NMR (400 MHz, CDCl₃) δ_{H} 4.17-4.07 (m, 1H), 1.90-1.84 (m, 3H), 1.67-1.61 (m, 9H), 1.26-1.24 (m, 8H), 1.13-1.09 (m, 6H), 0.97-0.89 (m, 6H), 0.77-0.76 (m, 1H), 0.76 (s, 2H), 0.73-0.71 (m, 6H).

### Synthesis of 44.4

To a solution of 44.3 (1.60 g, 4.41 mmol) in DCM (20 mL) was added PCC (1.90 g, 8.82 mmol) and silica gel (1.90 g). After stirred at RT for 1 h, the mixture was filtered, and the filter cake was washed with EtOAc (2×30 mL). The filtrate was concentrated and purified by silica gel chromatography (0-20% EtOAc in PE) to get 44.4 (500 mg, 31.4%). ¹H NMR (400 MHz, CDCl₃) δ_{H} 2.49-2.43 (m, 0.5H), 2.30-2.23 (m, 0.5H), 2.12 (d, J=3.2 Hz, 3H), 1.92-1.59 (m, 12H), 1.53-1.26 (m, 8H), 1.23-0.97 (m, 9H), 0.93-0.90 (m, 3H), 0.77-0.72 (m, 4H).

### Synthesis of 44.5

To a solution of 44.4 (150 mg, 0.416 mmol) in MeOH (10 ml) was added HBr (16.8 mg, 0.083 mmol, 40% in water) and Br₂ (79.7 mg, 0.50 mmol). After stirring at RT for 1 h, the reaction was quenched with sat. aq. NaHCO₃ (10 mL), then extracted with EtOAc (2×30 mL). The combined organic phase was washed with brine (30 mL), dried over anhydrous Na₂SO₄, filtered and concentrated to afford 44.5 (200 mg, crude), which was used directly to the next step.

### Synthesis of 44 & 45

To a solution of 44.5 (200 mg, 0.46 mmol) in acetone (10 mL) was added 1H-pyrazole-4-carbonitrile (63.5 mg, 0.68 mmol) and K₂CO₃ (63.8 mg, 0.455 mmol) and the mixture was stirred at RT for 16 h. Water (50 mL) was added and the mixture was extracted with EtOAc (3×50 mL). The combined organic layer was concentrated and the residue was purified by flash column (0-50% of EtOAc in PE) to give crude 44 and 45. The crude product mixture was further purified by pre-HPLC (Column: Welch Xtimate C18 150*25 mm*5 μm; Condition: water (0.225% TFA)-ACN; Begin B: 62; End B: 92; Gradient Time (min): 8.5; 100% B Hold Time (min): 2; Flow Rate(ml/min): 30; Injections: 6) to give 45 (14.5 mg, 7.1%). The crude 44 was triturated with PE/EtOAc (10/1, 11 mL) to afford 44 (39.6 mg, 19.3%).

44: ¹H NMR (400 MHz, CDCl₃)^{6H} 7.81 (d, J=6.0 Hz, 2H), 5.09-4.90 (m, 2H), 2.35-2.25 (m, 1H), 1.93-1.58 (m, 10H), 1.39-1.02 (m, 16H), 0.94 (s, 3H), 0.91-0.75 (m, 4H), 0.71 (s, 3H). LC-ELSD/MS: purity 99%; MS ESI calcd. for C₂₈H₄₂N₃O₂ [M+H]⁺ 452.3, found 452.3; MS ESI calcd. for C₂₈H₄₂N₃O₂Na [M+Na]⁺474.3, found 474.3; MS ESI calcd. for C₂₈H₄₀N₃O [M−H₂O+H]⁺ 434.3, found 434.3.

45: ¹H NMR (400 MHz, CDCl₃) δ_{H} 7.84 (d, J=9.2 Hz, 1H), 7.88-7.75 (m, 1H), 5.06-4.82 (m, 2H), 2.46 (d, J=4.8 Hz, 1H), 1.98-1.58 (m, 10H), 1.54-1.20 (m, 12H), 1.19 (s, 3H), 1.13-1.01 (m, 2H), 0.98 (s, 3H), 0.96-0.74 (m, 3H), 0.71 (s, 3H). LC-ELSD/MIS: purity 99%, analytic SFC: 100% de; MS ESI calcd. for C₂₅H₄₂N₃O₂Na [M+Na]r 474.3, found 474.3; MS ESI calcd. for C₂₈H₄₀N₃O [M−H₂O+H]r 434.3, found 434.3.

### Example 46: Synthesis of 1-(2-((1S,4aS,4bR,6aR,9S,11aS,11bR,13aS)-9-hydroxy-9-(methoxymethyl)-13a-methyloctadecahydro-1H-cyclohepta[a]phenanthren-1-yl)-2-oxoethyl)-1H-pyrazole-4-carbonitrile (46)

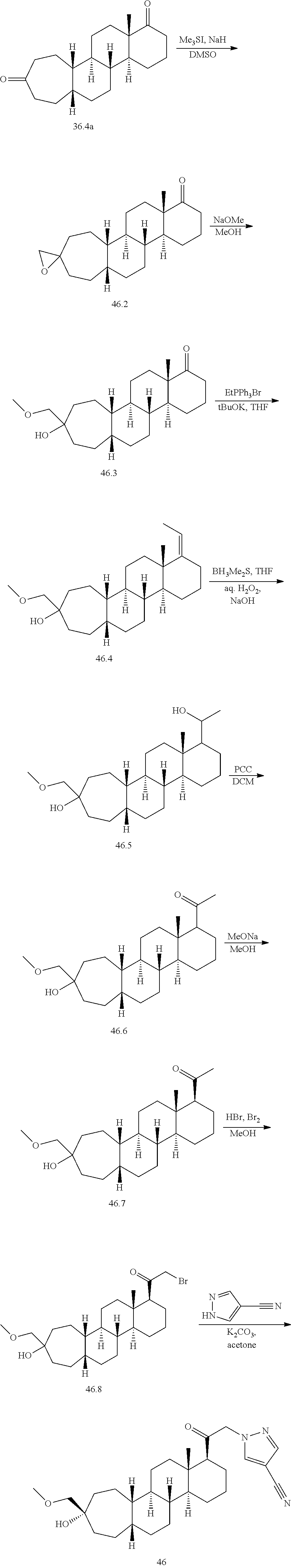

### Synthesis of 46.2

At 0° C., to a stirred solution of trimethylsulfonium iodide (1.61 g, 7.93 mmol) in DMSO (20 mL) and THF (10 mL) was add NaH (316 mg, 7.93 mmol, 60% in mineral oil) and the mixture was stirred at 0° C. for 1 h. The mixture was then added to a cooled (0° C.) solution of 36.4a (2.00 g, 6.61 mmol) in DMSO (10 mL) and stirred at 0 to 20° C. for 16 h. Water (200 mL) was added and the mixture was extracted with EtOAc (3×100 mL). The combined organic layer was washed with brine (100 mL×2), dried over anhydrous Na₂SO₄, filtered and concentrated. The residue was purified by flash column (0-30% of EtOAc in PE) to give 46.2 (1.40 g, 66.9%). ¹H NMR (400 MHz, CDCl3)) δ_{H} 3.11-2.98 (m, 1H), 2.86-2.78 (m, 1H), 2.69-2.50 (m, 2H), 2.43-2.31 (m, 1H), 2.25-2.06 (m, 1H), 2.01-1.57 (m, 10H), 1.55-1.27 (m, 6H), 1.23-0.84 (m, 8H).

### Synthesis of 46.3

To a solution of 46.2 (1.40 g, 4.42 mmol) in MeOH (15 mL) was added MeONa (2.38 g, 44.2 mmol) and the reaction mixture was stirred at 70° C. for 16 h. The reaction was cooled and quenched with saturated aq. NH₄Cl solution (200 mL), then extracted with EtOAc (3×100 mL). The combined organic phase was washed with saturated brine (2×50 mL), dried over anhydrous Na₂SO₄, filtered and concentrated. The residue was purified by flash column (0˜30% of EtOAc in PE) to give the product 46.3 (1.40 g, impure). ¹H NMR (400 MHz, CDCl₃) δ_{H} 3.40-3.35 (m, 3H), 3.25-3.13 (m, 2H), 2.71-2.54 (m, 1H), 2.32-2.14 (m, 1H), 1.95-1.59 (m, 9H), 1.58-1.28 (m, 9H), 1.22-0.75 (m, 9H).

### Synthesis of 46.4

To a solution of EtPPh₃Br (8.91 g, 24.0 mmol) in THE (50 mL) was added t-BuOK (2.69 g, 24.0 mmol) and the resulting mixture was warmed to 60° C. and stirred for 30 min. 46.3 (1.40 g, 4.01 mmol) was added in small portions while keeping the internal temperature below 70° C. The reaction mixture was stirred at 60° C. for 16 h to give a yellow suspension. After cooling, the reaction was quenched with 10% NH₄Cl aqueous solution (100 mL). The mixture was extracted with EtOAc (3×100 mL) and the combined organic layer was washed with brine (2×50 mL), dried over Na₂SO₄, filtered and concentrated. The residue was purified by flash column (0˜30% of EtOAc in PE) to give 46.4 (510 mg, impure). ¹H NMR (400 MHz, CDCl₃)⁶H 5.18-5.07 (m, 1H), 3.38 (s, 3H), 3.16 (s, 2H), 2.55-2.43 (m, 1H), 2.33-2.04 (m, 2H), 2.02-1.59 (m, 12H), 1.59-1.29 (m, 10H), 1.17-1.08 (m, 3H), 1.07-0.95 (m, 3H), 0.94-0.68 (m, 4H).

### Synthesis of 46.5

To a solution of 46.4 (510 mg, 1.41 mmol) in THE (20 mL) was added BH₃⁻Me₂S (428 mg, 564 μL, 5.64 mmol, 10 M) and the mixture was stirred at RT for 16 h. The mixture was cooled to 0° C. and to it was added ethanol (1.14 mL), followed by NaOH aqueous (2.82 mL, 5.0 M, 14.1 mmol). Hydrogen peroxide (1.41 mL, 10 M, 14.1 mmol) was added dropwise and the reaction mixture was warmed to 70° C. and stirred for 1 hour. The mixture was cooled to 15° C. and Na₂S₂O₃ (100 mL, sat. aq.) was added. The suspension was extracted with EtOAc (100 mL×3) and the combined organic layer was washed brine (2×50 mL), dried over Na₂SO₄, filtered and concentrated to give 46.5 (580 mg, crude). ¹H NMR (400 MHz, CDCl₃) δ_{H} 4.16-4.02 (m, 1H), 3.44-3.34 (m, 3H), 3.28-3.09 (m, 2H), 1.98-1.64 (m, 9H), 1.56-1.27 (m, 11H), 1.19-1.05 (m, 6H), 1.01-0.81 (m, 8H), 0.80-0.68 (m, 2H).

### Synthesis of 46.6

A solution of 46.5 (580 mg, 1.53 mmol), silica gel (1.50 g) and PCC (657 mg, 3.06 mmol) in DCM (20 mL) was stirred at RT for 1 h. The mixture was concentrated, and the residue was purified by flash column (0-15% of EtOAc in PE) to give 46.6 (380 mg, 65.4%). ¹H NMR (400 MHz, CDCl₃) δ_{H} 3.43-3.34 (m, 3H), 3.25-3.05 (m, 2H), 2.52-2.25 (m, 1H), 2.18-2.07 (m, 3H), 2.02-1.57 (m, 11H), 1.55-1.27 (m, 10H), 1.25-0.95 (m, 5H), 0.93 (s, 3H), 0.89-0.74 (m, 2H).

### Synthesis of 46.7

At 0° C., to a solution of 46.6 (380 mg, 1.00 mmol) in MeOH (20 mL) was added MeONa (1.08 g, 20.0 mmol). The reaction was warmed to 80° C. and stirred for 16 h. After cooling, the reaction mixture was poured into saturated NH₄Cl (100 mL) and the suspension was extracted with EtOAc (3×100 mL). The combined organic phase was washed with saturated brine (2×100 mL), dried over anhydrous Na₂SO₄, filtered and concentrated to give 46.7 (370 mg, 98.4%). ¹H NMR (400 MHz, CDCl₃) δ_{H} 3.44-3.35 (m, 3H), 3.29-3.12 (m, 2H), 2.35-2.24 (m, 1H), 2.13 (s, 3H), 2.00-1.59 (m, 10H), 1.56-1.29 (m, 10H), 1.25-0.95 (m, 6H), 0.92 (s, 3H), 0.90-0.73 (m, 2H).

### Synthesis of 46.8

To a solution of 46.7 (180 mg, 0.48 mmol) in MeOH (5 mL) was added HBr (19.3 mg, 0.096 mmol, 40% in water) and Br₂ (83.9 mg, 0.53 mmol). The mixture was stirred at RT for 2 h, then quenched with sat.aq NaHCO₃ (10 mL). Water (20 mL) was added and the suspension was extracted with EtOAc (3×30 mL). The combined organic phase was washed with brine (30 mL), dried over anhydrous Na₂SO₄, filtered, concentrated to afford 46.8 (190 mg, crude), which was used directly for the next step. ¹H NMR (400 MHz, CDCl₃) δ_{H} 4.07-3.76 (m, 2H), 3.42-3.35 (m, 3H), 3.27-3.04 (m, 2H), 2.80-2.48 (m, 1H), 1.99-1.57 (m, 11H), 1.55-1.28 (m, 10H), 1.23-0.97 (m, 5H), 0.95 (s, 3H), 0.91-0.72 (m, 2H).

### Synthesis of 46 & 46a

To a solution of 46.8 (190 mg, 0.42 mmol) in acetone (5 mL) was added 4-cyanopyrazole (46.5 mg, 0.50 mmol) and K₂CO₃ (58.4 mg, 0.42 mmol) and the mixture was stirred at RT for 16 h. Water (50 mL) was added and the mixture was extracted with EtOAc (3×50 mL). The organic layer was washed with brine (30 mL), dried over anhydrous Na₂SO₄, filtered, concentrated. The residue was purified by flash column (0˜45% of EtOAc in PE) to give 46 & 46a (200 mg, impure), which was further purified SFC (column: DAICEL CHIRALCEL OD (250 mm*30 mm*10 um); Mobile phase: A: CO₂ B: 0.1% NH₃H₂O EtOH; gradient: from 35% to 35% of B, Flow Rate (ml/min): 60) to give 46 (54.8 mg, 29.0%).

46: ¹H NMR (400 MHz, CDCl₃) δ_{H} 7.82 (s, 1H), 7.81 (s, 1H), 5.18-4.79 (m, 2H), 3.38 (s, 3H), 3.16 (s, 2H), 2.32 (dd, J=2.8, 12.4 Hz, 1H), 2.10 (s, 1H), 1.95-1.61 (m, 10H), 1.57-0.98 (m, 15H), 0.96 (s, 3H), 0.93-0.77 (m, 2H). LC-ELSD/MS purity: 98%, MS ESI calcd. for C₂₈H₄₁N₃O₃ [M+H]⁺ 468.3, found C₂₈H₄₁N₃O₃ [M−H₂O+H]⁺450.3.

### Example 47 & 48 & 49 & 50: Synthesis of 1-((1R,4aS,4bR,6aR,9S,11aS,11bR,13aS)-9-ethyl-9-hydroxy-13a-methyloctadecahydro-1H-cyclohepta[a]phenanthren-1-yl)ethanone (47) & Synthesis of 1-(2-((1R,4aS,4bR,6aR,9S,11aS,11bR,13aS)-9-ethyl-9-hydroxy-13a-methyloctadecahydro-1H-cyclohepta[a]phenanthren-1-yl)-2-oxoethyl)-1H-pyrazole-4-carbonitrile (48) & 1-((1S,4aS,4bR,6aR,9S,11aS,11bR,13aS)-9-ethyl-9-hydroxy-13a-methyloctadecahydro-1H-cyclohepta[a]phenanthren-1-yl)ethanone (49) & Synthesis of 1-(2-((1S,4aS,4bR,6aR,9S,11aS,11bR,13aS)-9-ethyl-9-hydroxy-13a-methyloctadecahydro-1H-cyclohepta[alphenanthren-1-yl)-2-oxoethyl)-1H-pyrazole-4-carbonitrile (50)

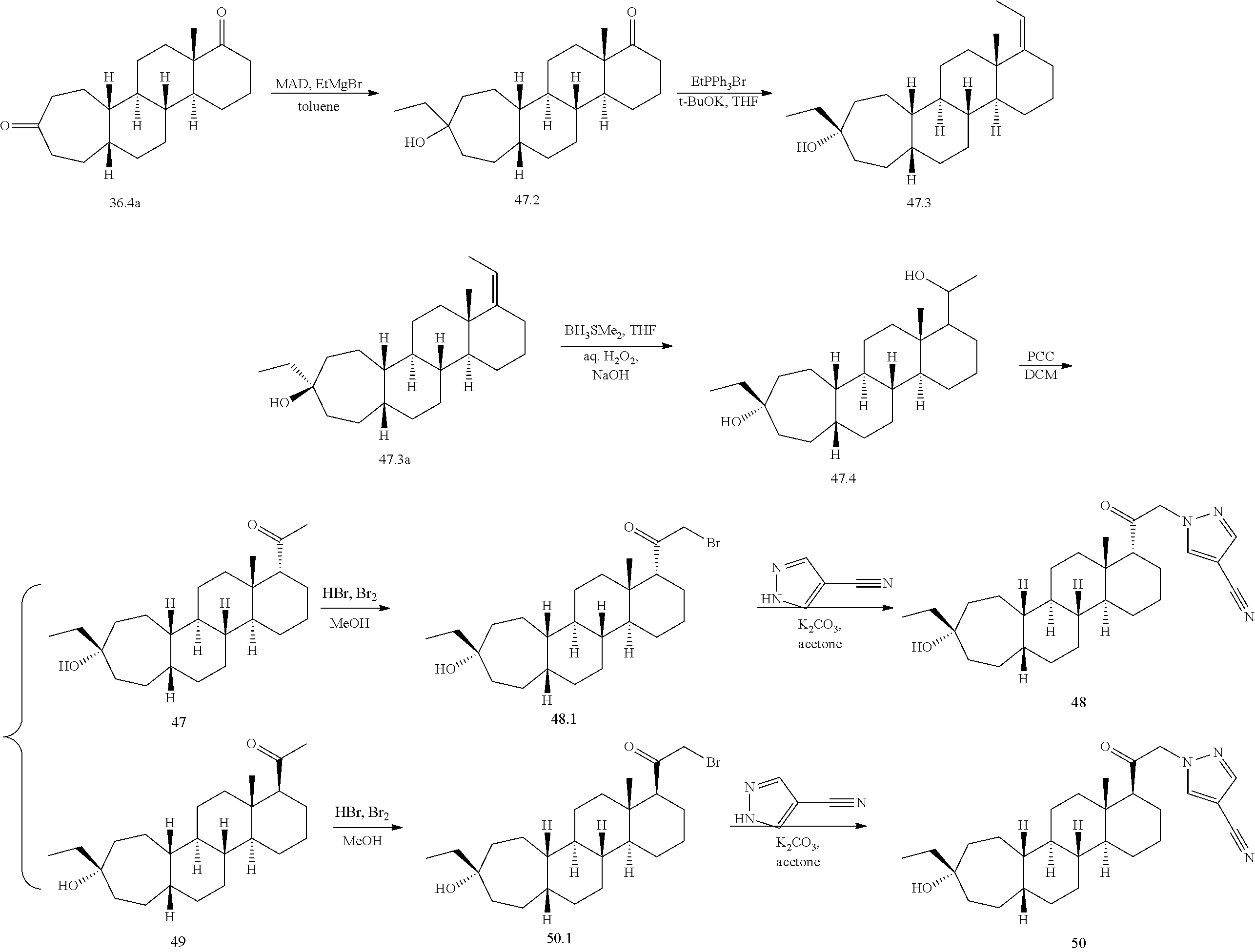

### Synthesis of 47.2

A fresh prepared MAD (19.8 mmol, in 20 mL toluene) solution was cooled to −70° C. and to it was added 36.4a (2.00 g, 6.61 mmol) in DCM (5 mL) dropwise. After stirring at −70° C. for 1 h, EtMgBr (8.80 mL, 26.4 mmol, 3M in ethyl ether) was added dropwise at −70° C. and the resulting solution was stirred at −70° C. for another 4 h. The reaction mixture was poured into citric acid (100 mL, 20%) and extracted with EtOAc (2×70 mL). The combined organic layers were washed with brine (100 mL), dried over Na₂SO₄, filtered and concentrated. The residue was purified by flash column (5˜30% EtOAc in PE) to give 47.2 (1.20 g, 54.6%). ¹H NMR (400 MHz, CDCl₃) δ_{H} 2.68-2.56 (m, 1H), 2.25-2.15 (m, 1H), 2.08-2.05 (m, 1H), 2.03-2.01 (m, 1H), 1.94-1.72 (m, 4H), 1.70-1.58 (m, 5H), 1.57-1.34 (m, 10H), 1.34-1.22 (m, 2H), 1.21-1.11 (m, 2H), 1.11-1.08 (i, 3H), 1.06-0.97 (m, 2H), 0.95-0.78 (m, 4H).

### Synthesis of 47.3 & 47.3a

To a solution of EtPPh₃Br (8.01 g, 21.6 mmol) in THE (20 mL) was added t-BuOK (2.41 g, 21.6 mmol). The resulting mixture was warmed to 40° C. and stirred for 1 h. 47.2 (0.20 g, 3.60 mmol) was added in small portions while keeping the internal temperature below 50° C. The reaction mixture was warmed to 65° C. and stirred for 16 h to give an orange suspension. The reaction was cooled to 15° C. and quenched with 10% NH₄Cl aqueous solution (80 mL). The layers were separated, and the aqueous layer was extracted with EtOAc (2×50 mL). The combined organic phase was concentrated, and the residue was purified by flash column (0˜5% of EtOAc in PE) to give 47.3 (366 mg, 29.5%) and 47.3a (392 mg, 31.6%).

47.3: ¹H NMR (400 MHz, CDCl₃) δ_{H} 5.20-5.10 (m, 1H), 2.56-2.44 (m, 1H), 2.24-2.08 (m, 1H), 2.00-1.71 (m, 6H), 1.69-1.58 (m, 8H), 1.54-1.32 (m, 9H), 1.19-1.04 (m, 6H), 1.03-0.95 (m, 2H), 0.94-0.87 (m, 6H)

47.3a: ¹H NMR (400 MHz, CDCl₃) δ_{H} 5.26-5.04 (m, 1H), 2.58-2.41 (m, 1H), 2.27-2.10 (m, 1H), 2.02-1.80 (m, 3H), 1.80-1.58 (m, 10H), 1.56-1.37 (m, 9H), 1.20-1.00 (m, 7H), 1.00-0.95 (m, 1H), 0.94-0.73 (m, 7H) Synthesis of 47.4

To a solution of 47.3 (366 mg, 1.06 mmol) in THE (5 mL) was added BH₃-Me₂S (0.96 mL, 10 M, 9.6 mmol) and the mixture was stirred at RT for 16 h. The mixture is cooled to 0° C. and EtOH (3.03 mL, 53.0 mmol) was added, followed by NaOH (10.6 mL, 5 M, 53.0 mmol). H₂O₂(5.30 mL, 10 M, 53.0 mmol) was added dropwise and the mixture was warmed to 78° C. and stirred for 2 h. After cooling. the reaction was quenched by Na₂SO₃ (30 mL, 10%) and extracted with EtOAc (2×20 mL). The combined organic layer was dried over Na₂SO₄, filtered and concentrated to give 47.4 (384 mg, crude). ¹H NMR (400 MHz, CDCl₃) δ_{H} 4.38-4.01 (m, 1H), 1.92-1.91 (m, 1H), 1.90-1.56 (m, 11H), 1.54-1.39 (m, 6H), 1.28-1.23 (m, 7H), 1.17-1.07 (m, 4H), 1.01-0.94 (m, 2H), 0.92-0.67 (m, 10H).

### Synthesis of 47&49

At 0° C., to a solution of 47.4 (380 mg, 1.04 mmol) in DCM (6 mL) was added silica gel (447 mg) and PCC (447 mg, 2.08 mmol). The mixture was stirred at 10° C. for 1.5 h and PE (3 mL) was added. The resulting mixture was filtered through a pad of silica gel and the filter cake was washed with DCM (3×6 mL). The filtrate was concentrated, and the residue was purified by silica gel chromatography (0-10% of EtOAc in PE) to give the 49 (86.0 mg, 22.9%) and 47 (86.0 mg, 22.9%).

47: ¹H NMR (400 MHz, CDCl₃) δ_{H} 2.49-2.44 (m, 1H), 2.13 (s, 3H), 1.85-1.58 (m, 10H), 1.54-1.33 (m, 12H), 1.31-1.21 (m, 2H), 1.15-1.03 (m, 4H), 0.93 (s, 3H), 0.91-0.86 (m, 4H), 0.86-0.81 (m, 1H).

49: ¹H NMR (400 MHz, CDCl₃) δ_{H} 2.34-2.26 (m, 1H), 2.14 (s, 3H), 1.87-1.60 (m, 8H), 1.53-1.19 (m, 13H), 1.16-0.94 (m, 6H), 0.93 (s, 3H), 0.92-0.87 (m, 4H), 0.87-0.74 (m, 2H).

### Synthesis of 50.1

To a solution of 49 (50.0 mg, 138 μmol) in MeOH (3 mL) was added HBr (40%, 5.50 mg, 27.6 μmol) and Br₂ (24.1 mg, 151 μmol). The mixture was stirred at RT for 2 h then quenched with saturated aqueous NaHCO₃ (10 mL). The suspension was extracted with EtOAc (2×20 mL) and the combined organic phase was washed with saturated brine (2×50 mL), dried over anhydrous Na₂SO₄, filtered and concentrated to give 50.1 (60.0 mg, crude).

### Synthesis of 50

To a solution of 50.1 (60.0 mg, 0.136 mmol) in acetone (4 mL) was added 4-cyanopyrazole (15.1 mg, 0.163 mmol) and K₂CO₃ (37.5 mg, 0.272 mmol). The mixture was stirred at RT for 4 h, water (20 mL) was added. The resultant suspension was extracted with EtOAc (2×20 mL) and the combined extract was dried over anhydrous Na₂SO₄, filtered and concentrated. The residue was purified by flash column (0-35% EtOAc in PE) to give 50 (26.3 mg, 42.8%). ¹H NMR (400 MHz, CDCl3) δ_{H} 7.82 (s, 1H), 7.81 (s, 1H), 5.06-4.93 (m, 2H), 2.37-2.28 (m, 1H), 1.91-1.60 (m, 10H), 1.50-1.20 (m, 11H), 1.18-1.00 (m, 6H), 0.96 (s, 3H), 0.93-0.76 (m, 6H). LC-ELSD/MS purity>99%, MS ESI calcd. for C₂₈H₄₁N₃O₂ [M+H-H₂O]⁺ 434.3, found 434.3.

### Synthesis of 48.1

To a solution of 47 (50.0 mg, 0.138 mmol) in methanol (3 mL) was added HBr (40%, 0.028 mmol) and Br₂ (24.1 mg, 0.151 mmol) and the dark solution was stirred at RT for 6 h. Saturated aqueous NaHCO₃ (10 mL) was added and the mixture was extracted with EtOAc (2×20 mL). The combined organic phase was washed with saturated brine (2×50 mL), dried over anhydrous Na₂SO₄, filtered and concentrated to give 48.1 (60.0 mg, crude).

### Synthesis of 48

To a solution of 48.1 (60.0 mg, 0.136 mmol) in acetone (4 mL) was added 4-cyanopyrazole (15.1 mg, 0.163 mmol) and K₂CO₃ (37.5 mg, 0.272 mmol), and the mixture was stirred at RT for 16 h. Water (20 mL) was added and the mixture was extracted with EtOAc (2×20 mL). The organic layer was separated, concentrated and purified by flash column (0˜35% EtOAc in PE) to give 48 (13.9 mg, 22.6%). ¹H NMR (400 MHz, CDCl3) δ_{H} 7.87 (s, 1H), 7.83 (s, 1H), 5.07-4.85 (m, 2H), 2.50-2.40 (m, 1H), 1.96-1.83 (m, 1H), 1.76-1.59 (m, 8H), 1.54-1.23 (m, 14H), 1.17-1.02 (m, 4H), 1.00 (s, 3H), 0.94-0.76 (m, 6H). LC-ELSD/MS purity>99%, MS ESI calcd. for C₂₅H₄₁N₃O₂ [M+H-H₂O]⁺ 434.3, found 434.3.

### Example 51: Synthesis of 1-(3-((1S,4aS,4bR,6aR,9S,11aS,11bR,13aS)-9-hydroxy-9,13a-dimethyloctadecahydro-1H-cyclohepta[a]phenanthren-1-yl)-3-oxopropyl)-1H-pyrazole-4-carbonitrile (51)

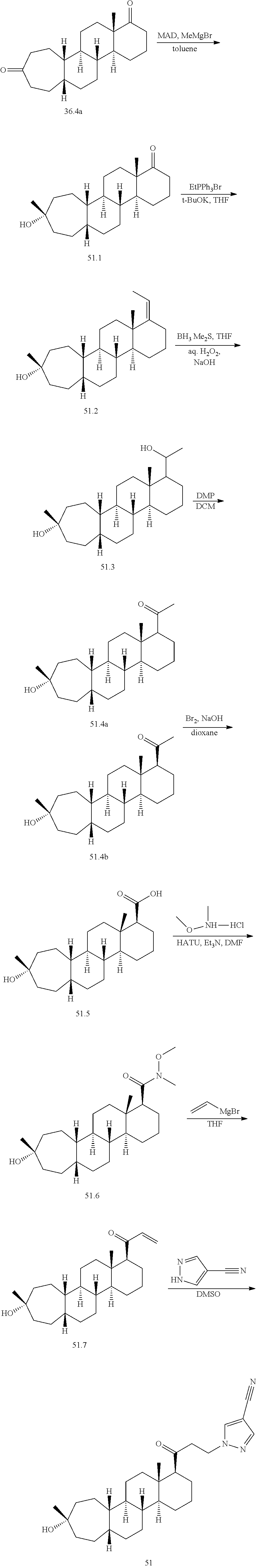

### Synthesis of 51.1

A fresh prepared MAD (32.6 mmol) solution in toluene (10 mL) was cooled to −70° C. and to it was added 36.4a (3.3 g, 10.9 mmol) in DCM (30 mL) dropwise. After stirring at −70° C. for 1 h, MeMgBr (14.5 mL, 43.6 mmol, 3M in ethyl ether) was added dropwise at −70° C. and the solution was stirred at −70° C. for another 4 h. The reaction mixture was poured into citric acid (50 mL, 20%) and extracted with EtOAc (3×100 mL). The combined organic layer was washed with brine (2×50 mL), dried over Na₂SO₄, filtered and concentrated. The residue was purified by flash column (0˜30% of EtOAc in PE) to give 51.1 (710 mg, 20.5%).

51.1: ¹H NMR (400 MHz, CDCl3) δ ppm 2.68-2.54 (m, 1H), 2.27-2.14 (m, 1H), 2.11-1.97 (m, 1H), 1.89-1.56 (m, 11H), 1.54-1.29 (m, 7H), 1.21 (s, 3H), 1.19-1.11 (m, 2H), 1.09 (s, 3H), 1.07-0.76 (m, 4H). LC-ELSD/MS purity 99%, MS ESI calcd. for C₂₁H₃₄O₂[M−H₂O+H]⁻ 301.3, found 301.3.

### Synthesis of 51.2

To a mixture of EtPPh₃Br (4.71 g, 12.7 mmol) in THF (15 mL) was added t-BuOK (1.42 g, 12.7 mmol) and the mixture was warmed to 60° C. and stirred for 30 min. 51.1 (680 mg, 2.13 mmol) was added in portions while keeping internal temperature below 60° C. The reaction was stirred at 60° C. for 16 h then cooled and quenched with 10% NH₄C₁ aqueous solution (100 mL). The suspension was extracted with EtOAc (3×50 mL) and the combined organic layer was washed brine (2×50 mL), dried over Na₂SO₄, filtered and concentrated. The residue was purified by flash column (0-30% of EtOAc in PE) to give 51.2 (780 mg, impure). ¹H NMR (400 MHz, CDCl3)^{6H} 5.22-5.08 (m, 1H), 2.59-2.43 (m, 1H), 2.30-2.07 (m, 1H), 2.02-1.58 (m, 14H), 1.56-1.28 (m, 7H), 1.21 (s, 3H), 1.16-1.03 (m, 5H), 0.93 (s, 4H), 0.89-0.69 (m, 2H).

### Synthesis of 51.3

To a solution of 51.2 (780 mg, 2.35 mmol) in THE (20 mL) was added BH₃⁻Me₂S (714 mg, 0.94 mL, 10 M, 9.40 mmol) and the mixture was stirred at RT for 16 h. After cooling to 0° C., ethanol (2.35 mL) was added, followed by aqueous NaOH (4.70 mL, 5.0 M, 23.5 mmol). Hydrogen peroxide (2.35 mL, 10 M, 23.5 mmol) was added dropwise and the reaction mixture was heated to 70° C. and stirred for 1 h. The mixture was cooled to 15° C. and Na₂S₂O₃ (100 mL, sat.aq.) was added. The resultant suspension was extracted with EtOAc (100 mL×3). The combined organic layer was washed with brine (2×50 mL), dried over Na₂SO₄, filtered and concentrated to give 51.3 (1.1 g, crude). ¹H NMR (400 MHz, CDCl3) δ_{H} 4.43-3.97 (m, 1H), 1.95-1.31 (m, 25H), 1.19-1.03 (m, 6H), 1.02-0.81 (m, 9H), 0.78-0.75 (m, 2H).

### Synthesis of 51.4a & 51.4b

At 0° C., to a solution of 51.3 (1 g, 2.86 mmol) in DCM (50 mL) was added Dess-Martin Reagent (2.42 g, 5.72 mmol). The mixture was warmed to RT and stirred for 30 min. The reaction was quenched with saturated aqueous NaHCO₃ (100 mL). The layers was separated and DCM layer was washed with saturated NaHCO₃/Na₂S₂O₃ aqueous solution (1:1, 2×100 mL), brine (2×50 mL), dried over Na₂SO₄, filtered and concentrated to give 51.4b (500 mg, crude) and 51.4a (19.5 mg, crude).

51.4b: ¹H NMR (400 MHz, CDCl3) δ_{H} 2.30 (dd, J=3.2, 12.8 Hz, 1H), 2.14 (s, 3H), 1.87-1.58 (m, 12H), 1.51-1.28 (m, 7H), 1.22 (s, 3H), 1.17-0.95 (m, 6H), 0.93 (s, 3H), 0.91-0.69 (m, 3H). LC-ELSD/MS purity 99%, MS ESI calcd. for C₂₃H₃₈O₂[M−H₂O+H]⁻ 329.3, found 329.3.

51.4a: ¹H NMR (400 MHz, CDCl3) δ_{H} 2.49-2.26 (m, 1H), 2.17-2.09 (m, 3H), 1.93-1.58 (m, 10H), 1.58-1.27 (m, 12H), 1.23-1.19 (m, 3H), 1.17-0.96 (m, 4H), 0.94-0.92 (m, 3H), 0.88-0.75 (m, 2H).

### Synthesis of 51.5

Br₂ (714 mg, 4.47 mmol) was added dropwise to an aqueous NaOH solution (1.19 g, 6.00 mL, 30.0 mmol, 5 M). The mixture was stirred at RT for 20 min and added to a solution of 51.4b (350 mg, 1.00 mmol) in dioxane (10 mL) and water (2.5 mL). The resulting mixture was stirred at RT for 16 h. Saturated aqueous Na₂SO₃ (30 mL) solution was added and the mixture was stirred at 70° C. for 1 hour. The mixture was cooled and HCl (3 M in water, 50 mL) was added to adjust the pH to 2, and stirred for 10 min. The precipitated solid was filtered out, washed with water (2×10 mL) and dried to give 51.5 (390 mg, crude). 1H NMR (400 MHz, CDCl₃) δ_{H} 2.20-2.09 (m, 1H), 1.85-1.64 (m, 11H), 1.52-1.25 (m, 10H), 1.22 (s, 3H), 1.13-0.97 (m, 5H), 0.95 (s, 3H), 0.90-0.75 (m, 3H). LC-ELSD/MS purity: 100%, MS ESI calcd. for C₂₂H₃₆O₃[M−H₂O+H]⁺331.3, found 331.2.

### Synthesis of 51.6

To a solution of 51.5 (390 mg, 1.11 mmol) in DMF (5 mL) was added N, O-dimethylhydroxylamine hydrochloride (433 mg, 4.44 mmol), HATU (843 mg, 2.22 mmol) and Et₃N (1.12 g, 11.1 mmol). The reaction mixture was stirred at RT for 16 h. Water (20 mL) was added and the mixture was extracted with ethyl acetate (30 mL×3). The combined organic phase was washed with brine (50 mL×2), dried over Na₂SO₄, filtered and concentrated. The residue was purified by flash column (15-45% of EtOAc in PE) to give 51.6 (450 mg, impure). ¹H NMR (400 MHz, CDCl₃) δ_{H} 3.66 (s, 3H), 3.16 (s, 3H), 2.71-2.44 (m, 1H), 1.84-1.45 (m, 21H), 1.21 (s, 3H), 1.11-1.04 (m, 3H), 1.02 (s, 3H), 1.01-0.68 (m, 5H).

### Synthesis of 51.7

A solution of 51.6 (450 mg, 1.14 mmol) in THF (5 mL) was cooled to 0° C. Vinylmagnesium bromide (3.25 mL, 0.7 M, 2.28 mmol) was added and the mixture was stirred at RT for 2 h. The reaction was quenched with 10% NH₄Cl (100 ml) and extracted with EtOAc (2×50 mL). The combined organic phase was washed with saturated NaCl (2×50 mL), dried over Na₂SO₄, filtered and concentrated. The residue was purified by flash column (0˜30% EtOAc in PE) to give 51.7 (100 mg, 24.4%).

### Synthesis of 51

A solution of 51.7 (100 mg, 0.279 mmol), 1-methyl-1H-imidazole (67.7 mg, 0.836 mmol) and 1H-pyrazole-4-carbonitrile (51.9 mg, 0.558 mmol) in DMSO (5 mL) was stirred at 70° C. for 16 h. After cooling, the reaction mixture was poured into H₂O (30 mL) and extracted with EtOAc (30 mL×3). The combined organic layer was washed with brine (30 mL×2), dried over Na₂SO₄, filtered and concentrated. The residue was purified by flash column (0˜30% EtOAc in PE) to give 51 (10.5 mg, 8.40%). ¹H NMR (400 MHz, CDCl₃)⁶H 7.89 (s, 1H), 7.75 (s, 1H), 4.45-4.32 (m, 2H), 3.11-2.92 (m, 2H), 2.30-2.15 (m, 1H), 1.87-1.67 (m, 5H), 1.53-1.40 (m, 7H), 1.36-1.24 (m, 5H), 1.21 (s, 3H), 1.17-0.90 (m, 9H), 0.86 (s, 3H), 0.82-0.73 (m, 2H). LC-ELSD/MS purity: 100%, MS ESI calcd. for C₂₈H₄₁N₃O₂ [M−H₂O+H]⁻ 434.3, found 434.3.

### Example 52 & 53: Synthesis of 1-(2-((1S,4aS,4bR,6aR,9S,11aS,11bS,13aS)-9-hydroxy-9,11a,13a-trimethyloctadecahydro-1H-cyclohepta[a]phenanthren-1-yl)-2-oxoethyl)-1H-pyrazole-4-carbonitrile (52) & Synthesis of 1-((1S,4aS,4bR,6aR,9S,11aS,11bS,13aS)-9-hydroxy-9,11a,13a-trimethyloctadecahydro-1H-cyclohepta[a]phenanthren-1-yl)ethanone (53)

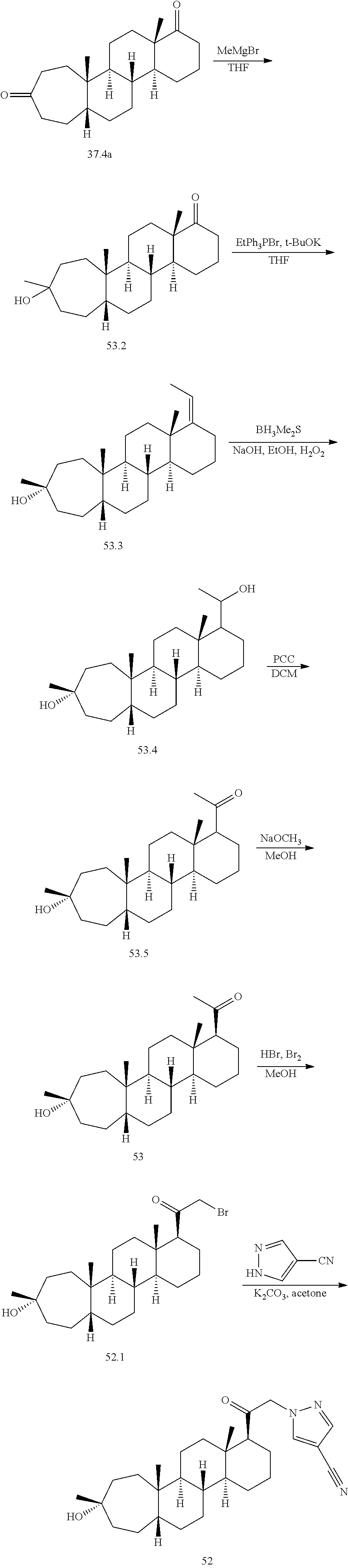

### Synthesis of 53.2

At 0° C., to a solution of 37.4a (1.00 g, 3.15 mmol) in THF (15 mL) was added MeMgBr (1.26 mL, 3.78 mmol, 3M) dropwise. The mixture was stirred at RT for 16 h to give a yellow solution. The reaction was quenched with saturated aq. NH₄Cl solution (50 mL) and extracted with EtOAc (3×50 mL). The combined organic phase was washed with saturated brine (50 mL), dried over anhydrous Na₂SO₄, filtered and concentrated to give the product 53.2 (1.10 g, crude).

### Synthesis of 53.3

To a suspension of Ph₃PEtBr (13.3 g, 36.0 mmol) in anhydrous THF (40 mL) was added t-BuOK (4.03 g, 36.0 mmol). The mixture was warmed to 60° C. and stirred for 30 min. A solution of 53.2 (2.00 g, 6.01 mmol) in anhydrous THF (10 mL) was added dropwise and the mixture was stirred at 60° C. for another 16 h. After cooling, the reaction mixture was poured into saturated NH₄Cl (50 mL) then stirred for 10 min. The resultant suspension was extracted with EtOAc (2×150 mL) and the combined organic phase was washed with saturated brine (2×100 mL), filtered and concentrated. The residue was purified by flash column (0-50% of EtOAc in PE) to give product 53.3 (800 mg, 38.6%).

53.3: ¹H NMR (400 MHz, CDCl₃) δ 5.21-5.10 (m, 1H), 1.86-1.59 (m, 10H), 1.58-1.38 (m, 9H), 1.38-1.18 (m, 10H), 1.15-1.02 (m, 5H), 0.93 (m, J=4.8 Hz, 5H).

### Synthesis of 53.4

To a solution of 53.3 (800 mg, 2.32 mmol) in THF (5 mL) was added BH₃-Me₂S (1.16 mL, 10 M, 11.6 mmol) and the mixture was warmed to 45° C. and stirred for 1 h. The mixture is cooled to 15° C. and ethanol was added (2.13 g, 46.4 mmol), followed by NaOH aqueous solution (9.28 mL, 5.0 M, 46.4 mmol). H₂O₂(4.64 mL, 10 M, 46.4 mmol) was added dropwise and the mixture was then heated to 70° C. and stirred for 1 h. The mixture was cooled and poured into water (50 mL) and extracted with EtOAc (2×50 mL). The combined extract was washed with saturated brine (2×50 mL), drive over anhydrous Na₂SO₄, filtered and concentrated to give 53.4 (800 mg, crude).

### Synthesis of 53.5

To a solution of 53.4 (800 mg, 2.20 mmol) in DCM (10 mL) was added silica gel (2.00 g) and PCC (946 mg, 4.40 mmol). The mixture was stirred at RT for 2 h to give a yellow suspension. The mixture was filtered, and the filter cake was washed with DCM (3×20 mL). The filtrate was concentrated, and the residue was purified by flash column (0-40% of EtOAc in PE) to give 53.5 (450 mg, 56.7%). ¹H NMR (400 MHz, CDCl₃) δ_{H} 2.51-2.25 (m, 1H), 2.13 (d, J=3.2 Hz, 3H), 1.90-1.69 (m, 5H), 1.52-1.41 (m, 5H), 1.35-1.10 (m, 14H), 1.00-0.68 (m, 12H).

### Synthesis of 53

To a solution of 53.5 (500 mg, 1.38 mmol) in MeOH (10 mL) was added MeONa (1.10 g, 20.7 mmol) and the reaction mixture was stirred at 70° C. for 16 h. The mixture was cooled and poured into water (20 mL) and stirred for 10 min. The suspension was extracted with EtOAc (3×20 mL) and the combined organic phase was washed with saturated brine (2×20 mL), dried over anhydrous Na₂SO₄, filtered and concentrated to give 53 (280 mg, 56.3%). ¹H NMR (400 MHz, CDCl₃) δ 2.32-2.25 (m, 1H), 2.13 (s, 3H), 1.92-1.61 (m, 9H), 1.53-1.44 (m, 4H), 1.33-1.18 (m, 9H), 1.17-0.98 (m, 5H), 0.93-0.88 (m, 6H), 0.86-0.68 (m, 3H). LC-ELSD/MS purity: 100%, MS ESI calcd. for C₂₄H₃₉O [M−H₂O+H]⁺343.3, found 343.3.

### Synthesis of 52.1

At 0° C., to a solution of 53 (140 mg, 0.39 mmol) and HBr (5.00 mg, 0.02 mmol, 40%) in MeOH (5 mL) was added Br₂ (74.5 mg, 0.46 mmol). The mixture was stirred at RT for 2 h then poured into saturated NaHCO₃ (20 mL). The suspension was extracted with EtOAc (3×20 mL) and the combined organic layer was washed with saturated brine (30 mL), dried over anhydrous Na₂SO₄, filtered and concentrated to give 52.1 (160 mg, crude).

### Synthesis of 52

To a solution of 52.1 (160 mg, 0.36 mmol) in acetone (5 ml) was added K₂CO₃ (100 mg, 0.728 mmol) and 1H-pyrazole-4-carbonitrile (50.8 mg, 0.54 mmol), and the mixture was stirred at RT for 2 h. The mixture was poured into water (20 mL) and extracted with EtOAc (2×20 ml), dried over anhydrous Na₂SO₄, filtered and concentrated. The residue was purified by flash column (0-40% of EtOAc in PE) to give product 52 (42.0 mg, 25.6%). 1H NMR (400 MHz, CDCl₃) δ_{H} 7.87-7.77 (m, 2H), 5.09-4.89 (m, 2H), 2.35-2.24 (m, 1H), 1.89-1.60 (m, 10H), 1.58-1.40 (m, 9H), 1.34-1.09 (m, 11H), 0.96-0.87 (m, 6H). LCMS purity>99%, MS ESI calcd. for C₂₅H₄₀N₃O [M−H₂O+H]⁺434.3, found 434.3.

### Example 54: Synthesis of 1-(3-((1S,4aS,4bR,6aS,9S,11aS,11bS,13aS)-9-hydroxy-9,11a,13a-trimethyloctadecahydro-1H-cyclohepta[a]phenanthren-1-yl)-3-oxopropyl)-1H-pyrazole-4-carbonitrile (54)

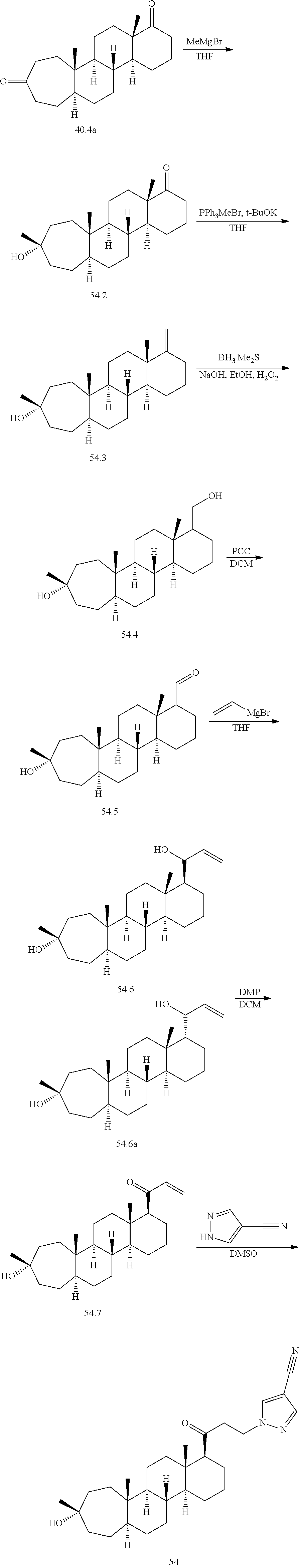

### Synthesis of 54.2

At 0° C., to a solution of 40.4a (4.00 g, 12.6 mmol) in THE (40 mL) was added MeMgBr (5.03 mL, 15.1 mmol, 3M) and the mixture was stirred at RT for 16 h. The mixture was poured into saturated NH₄Cl (50 mL), stirred for 10 min and extracted with EtOAc (2×50 mL). The combined organic phase was washed with saturated brine, filtered and concentrated. The residue was purified by flash column (0-30% of EtOAc in PE) to give 54.2 (1.20 g, 28.7%).

54.2: ¹H NMR (400 MHz, CDCl₃) O_{H} 2.68-2.53 (m, 1H), 2.22-2.13 (m, 1H), 2.07-2.02 (m, 1H), 1.90-1.65 (m, 7H), 1.57-1.40 (m, 5H), 1.39-1.26 (m, 6H), 1.20 (s, 3H), 1.19-1.09 (m, 4H), 1.07 (s, 3H), 0.89-0.80 (m, 2H), 0.73 (s, 3H).

### Synthesis of 54.3

To a solution of MePh₃PBr (3.85 g, 10.8 mmol) in THF (40 mL) was added t-BuOK (1.21 g, 10.8 mmol). The mixture was warmed to 50° C. and stirred for 1 h. A solution of 54.2 (1.20 g, 3.60 mmol) in THF (10 mL) was added and the reaction mixture was stirred at 50° C. for another 16 h. The mixture was cooled and poured into saturated NH₄Cl (100 mL). The suspension was extracted with EtOAc (3×100 mL) and the combined organic layer was washed with saturated brine (100 mL), dried over anhydrous Na₂SO₄, filtered and concentrated. The residue was purified by flash column (0-30% of EtOAc in PE) to give 54.3 (700 mg, 58.8%). ¹H NMR (400 MHz, CDCl₃) δ_{H} 4.57 (d, J=10.8 Hz, 2H), 2.39-2.25 (m, 1H), 2.13-2.03 (m, 1H), 1.90-1.75 (m, 4H), 1.74-1.61 (m, 4H), 1.59-1.51 (m, 2H), 1.50-1.39 (m, 1H), 1.38-1.22 (m, 8H), 1.20 (s, 3H), 1.14-1.02 (m, 3H), 0.94 (s, 3H), 0.90-0.75 (m, 3H), 0.74 (s, 3H).

### Synthesis of 54.4

To a solution of 54.3 (700 mg, 2.11 mmol) in THF (10 mL) was added BH₃-Me₂S (0.632 ml, 6.32 mmol, 10 M) and the mixture was stirred at RT for 1 h. The resulting mixture was cooled to 0° C. and ethanol (970 mg, 21.1 mmol) was added, followed by aqueous NaOH solution (4.22 mL, 5M, 21.1 mmol). H₂O₂ (2.11 mL, 10 M, 21.1 mmol) was added dropwise and the mixture was heated to 80° C. and stirred for 1 h. The mixture was cooled and poured into saturated Na₂S₂O₃ aqueous solution (50 mL). The suspension was stirred for 30 min and extracted with EtOAc (2×100 mL). The combined organic phase was washed with saturated brine (100 mL), dried over anhydrous Na₂SO₄ and concentrated to give 54.4 (1.00 g, crude). ¹H NMR (400 MHz, CDCl₃) δ_{H} 3.98-3.81 (m, 1H), 3.71-3.58 (m, 1H), 3.29 (s, 1H), 1.88-1.78 (m, 3H), 1.70-1.64 (m, 4H), 1.57-1.51 (m, 3H), 1.49-1.35 (m, 2H), 1.33-1.22 (m, 9H), 1.20 (s, 3H), 1.15-1.07 (m, 4H), 0.98-0.94 (m, 3H), 0.93-0.86 (m, 1H), 0.83-0.75 (m, 2H), 0.72 (s, 3H).

### Synthesis of 54.5

To a solution of 54.4 (800 mg, 2.29 mmol) in DCM (10 mL) was added silica gel (2.00 g) and PCC (984 mg, 4.58 mmol), and the mixture was stirred at RT for 1 h. The suspension was filtered, and the filter cake was washed with DCM (2×100 mL). The filtrate was concentrated to give 54.5 (800 mg, crude). ¹H NMR (400 MHz, CDCl₃) δ_{H} 10.20-9.75 (m, 1H), 2.04 (s, 3H), 1.90-1.81 (m, 4H), 1.75-1.65 (m, 5H), 1.64-1.54 (m, 6H), 1.25 (s, 8H), 1.16-1.09 (m, 3H), 1.00-0.89 (m, 5H), 0.72 (s, 3H).

### Synthesis of 54.6 & 54.6a

To a solution of 54.5 (800 mg, 2.30 mmol) in THF (10 mL) was added vinylmagnesium bromide (9.84 mL, 2.93 mmol, 0.7M) and the mixture was stirred at RT for 4 h. The mixture was poured into saturated NH₄Cl (100 mL) and stirred for 10 min. The suspension was extracted with EtOAc (2×100 mL) and the combined organic phase was washed with saturated brine, filtered and concentrated. The residue was purified by flash column (0-30% of EtOAc in PE) to give 54.6 (200 mg, 23.2%) and 54.6a (180 mg, 20.9%).

54.6: ¹H NMR (400 MHz, CDCl₃) δ_{H} 5.94-5.79 (m, 1H), 5.21-5.04 (m, 2H), 4.78 (s, 1H), 4.53 (s, 1H), 1.98-1.91 (m, 1H), 1.90-1.76 (m, 4H), 1.74-1.63 (m, 5H), 1.58-1.49 (m, 4H), 1.48-1.39 (m, 3H), 1.20 (s, 3H), 1.18-0.96 (m, 7H), 0.93 (s, 3H), 0.89-0.75 (m, 4H), 0.72 (s, 3H).

54.6a: ¹H NMR (400 MHz, CDCl₃) δ_{H} 5.97-5.87 (m, 1H), 5.27-5.09 (m, 2H), 4.63 (s, 1H), 1.89-1.82 (m, 3H), 1.75-1.65 (m, 6H), 1.57-1.50 (m, 5H), 1.27-1.24 (m, 7H), 1.20 (s, 3H), 1.13-1.06 (m, 4H), 0.95 (s, 3H), 0.86-0.76 (m, 4H), 0.73 (s, 3H).

### Synthesis of 54.7

To a solution of 54.6 (200 mg, 0.53 mmol) in DCM (5 mL) was added Dess-Martin Reagent (678 mg, 1.6 mmol). The mixture was stirred at RT 10 min then quenched with saturated NaHCO₃/Na₂S₂O₃ aqueous solution (1:1, 30 mL). The layers were separated, and the organic layer was washed with saturated NaHCO₃/Na₂S₂O₃ aqueous solution (1:1, 30 mL), dried over Na₂SO₄, filtered and concentrated. The residue was purified by flash column (0˜30% of EtOAc in PE) to give 54.7 (70 mg, 35.3%). ¹H NMR (400 MHz, CDCl₃)^{6H} 6.40 (dd, J=10.4, 17.2 Hz, 1H), 6.17 (d, J=17.2 Hz, 1H), 5.65 (d, J=10.4 Hz, 1H), 2.53 (dd, J=3.2, 12.4 Hz, 1H), 1.90-1.72 (m, 5H), 1.71-1.51 (m, 10H), 1.44-1.39 (m, 1H), 1.32-1.29 (m, 2H), 1.19 (s, 3H), 1.15-1.05 (m, 3H), 1.04-0.91 (m, 2H), 0.89 (s, 3H), 0.88-0.72 (m, 4H), 0.70 (s, 3H).

### Synthesis of 54

A solution of 54.7 (70.0 mg, 0.188 mmol), 1-methyl-1H-imidazole (46.2 mg, 0.563 mmol) and 1H-pyrazole-4-carbonitrile (34.9 mg, 0.376 mmol) in DMSO (5 mL) was stirred at 70° C. for 3 h. The mixture was cooled and poured into saturated brine (30 mL). The resultant suspension was extracted with EtOAc (3×30 mL) and the combined extract was washed with saturated brine (2×50 mL), dried over anhydrous Na₂SO₄, filtered and concentrated. The residue was purified by flash column (0-60% of EtOAc in PE) to give 54 (40.0 mg, impure), which was further purified by prep-HPLC (Column: YMC Triart C18 150*25 mm*5 um; Condition: water (10 mM NH₄HCO₃)-ACN; Begin B: 75; End B: 100; Gradient Time (min): 9.5; 100% B Hold Time (min): 2) to give 54 (19.8 mg, 49.6%). ¹H NMR (400 MHz, CDCl₃) δ_{H} 7.89 (s, 1H), 7.75 (s, 1H), 4.46-4.27 (m, 2H), 3.18-2.90 (m, 2H), 2.21 (dd, J=2.8, 12.4 Hz, 1H), 1.91-1.73 (m, 3H), 1.71-1.59 (m, 4H), 1.56-1.49 (m, 2H), 1.38-1.21 (m, 7H), 1.20 (s, 3H), 1.20-1.01 (m, 7H), 0.99-0.86 (m, 1H), 0.83 (s, 3H), 0.81-0.71 (m, 3H), 0.69 (s, 3H).

### Example 55: Synthesis of 1-(2-((1S,4aS,4bR,6aR,9S,11aS,11bS,13aS)-9-hydroxy-9-(methoxymethyl)-11a,13a-dimethyloctadecahydro-1H-cyclohepta[a]phenanthren-1-yl)-2-oxoethyl)-1H-pyrazole-4-carbonitrile (55)

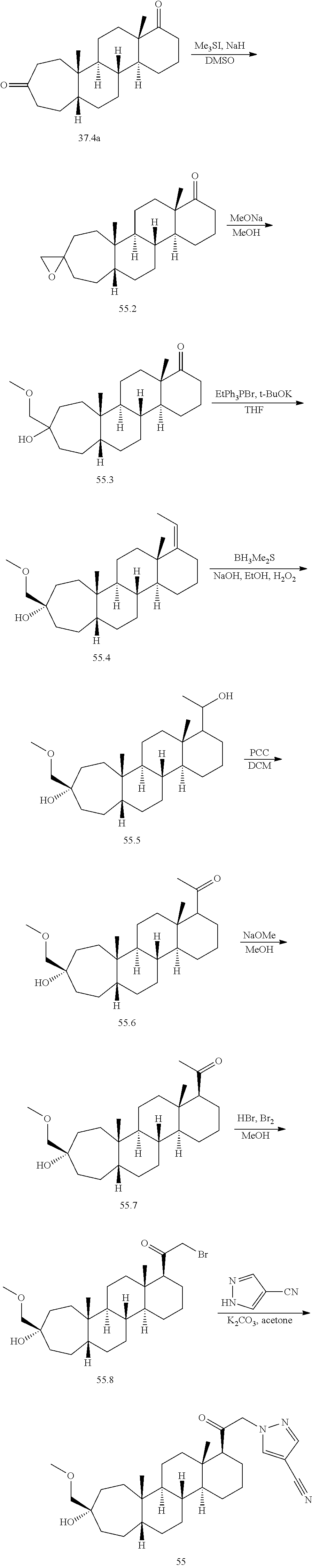

### Synthesis of 55.2

At 0° C., to a stirred solution of Me₃SI (2.89 g, 14.17 mmol) in DMSO (40 mL) and THF (40 mL) was added NaH (566 mg, 14.17 mmol, 60% in oil) in several small portions. The reaction mixture was stirred at RT for 1 h then added to a solution of 37.4 (3.00 g, 9.48 mmol) in THF (40 mL). After stirring at RT for 16 h, the mixture was poured into ice-water (200 mL) and extracted with EtOAc (2×100 mL). The combined organic phase was washed with water (2×100 mL), brine (100 mL), dried over anhydrous Na₂SO₄, filtered, and concentrated to give the product 55.2 (3.60 g, crude).

### Synthesis of 55.3

To a solution of 55.2 (3.60 g, 10.89 mmol) in MeOH (30 mL) was added MeONa (21.74 mL, 5 M in MeOH), 108.8 mmol) and the mixture was stirred at 60° C. for 16 h. After cooling, the reaction mixture was poured into saturated NH₄Cl (150 mL). The suspension was extracted with EtOAc (3×100 mL) and the combined organic layer was washed with saturated brine (100 mL), dried over anhydrous Na₂SO₄, filtered and concentrated. The residue was purified by flash column (0-10% of EtOAc in PE) to give 55.3 (2.00 g, impure).

### Synthesis of 55.4

To a suspension of Ph₃PEtBr (12.2 g, 32.88 mmol) in anhydrous THE (20 mL) was added t-BuOK (3.69 g, 32.88 mmol). The reaction mixture was warmed to 50° C. and stirred for 30 min. A solution of 55.3 (2.00 g, 5.52 mmol) in anhydrous THE (20 mL) was added dropwise and the mixtures was stirred at 50° C. for another 16 h. The mixture was cooled, poured into saturated NH₄Cl (200 mL) and stirred for 10 min. The suspension was extracted with EtOAc (2×100 mL) and the combined extract was washed with saturated brine, filtered and concentrated. The residue was purified by flash column (0-10% of EtOAc in PE) to give 55.4 (840 mg, 40.6%) and 55.4a (330 mg, 15.9%).

55.4: ¹H NMR (400 MHz, CDCl₃) δ_{H} 5.10-5.19 (m, 1H), 3.35-3.41 (m, 3H), 3.11-3.20 (m, 2H), 2.49 (br d, J=13.80 Hz, 1H), 1.78-2.02 (m, 4H), 1.64-1.77 (m, 5H), 1.54-1.61 (m, 7H), 1.29-1.53 (m, 6H), 1.01-1.20 (m, 6H), 0.87-0.93 (m, 5H), 0.69-0.85 (m, 2H).

### Synthesis of 55.5

To a solution of 55.4 (840 mg, 2.24 mmol) in THE (10 mL) was added BH₃-Me₂S (1.11 mL, 11.2 mmol, 10 M) and the mixture was stirred at RT for 1 h. The resulting mixture was cooled to 15° C. and ethanol (1.03 g, 22.4 mmol) added, followed by NaOH aqueous solution (4.47 mL, 5M, 22.4 mmol). The mixture was cooled to 0° C. and H₂O₂(2.23 mL, 10 M, 22.4 mmol) was added dropwise. After the addition, the mixture was heated to 80° C. and stirred for 1 h. The reaction mixture was cooled, added to saturated aqueous Na₂S₂O₃ solution (100 mL) and stirred for 30 min. The suspension was extracted with EtOAc (100 mL) and the combined organic phase was washed with saturated brine (2×100 mL), dried over anhydrous Na₂SO₄, filtered and concentrated to give 55.5 (930 mg, crude).

### Synthesis of 55.6

To a solution of 55.5 (930 mg, 2.36 mmol) in DCM (50 mL) was added silica gel (2.00 g) and PCC (1.01 g, 4.72 mmol). The mixture was stirred at RT for 1 h then filtered. The filter cake was washed with DCM (2×30 mL) and the filtrate was concentrated. The residue was purified by flash column (0%-15% of EtOAc in PE) to give 55.6 (630 mg, 68.4%). ¹H NMR (400 MHz, CDCl₃) δ_{H} 3.36 (d, J=3.26 Hz, 3H), 3.11-3.18 (m, 2H), 2.25-2.46 (m, 1H), 2.12 (d, J=4.27 Hz, 3H), 2.03 (s, 2H), 1.77-2.00 (m, 3H), 1.33-1.72 (m, 14H), 1.29 (br s, 2H), 1.00-1.20 (m, 4H), 0.84-0.93 (m, 7H), 0.74-0.83 (m, 1H).

### Synthesis of 55.7

A solution of 55.6 (630 mg, 1.61 mmol) and MeONa (1.30 g, 24.1 mmol) in MeOH (30 mL) was stirred at 70° C. for 48 h. The mixture was cooled and poured into water (50 mL). The suspension was extracted with EtOAc (3×30 mL) and the combined organic layer was washed with saturated brine (50 mL), dried over anhydrous Na₂SO₄, filtered and concentrated. The residue was purified by flash column (0-10% of EtOAc in PE) to give 55.7 (620 mg, 98.7%). ¹H NMR (400 MHz, CDCl₃) an 3.30-3.41 (m, 3H), 3.09-3.19 (m, 2H), 2.23-2.46 (m, 1H), 2.08-2.15 (m, 3H), 1.61-1.98 (m, 10H), 1.29-1.61 (m, 10H), 1.00-1.19 (m, 4H), 0.91-0.99 (m, 1H), 0.84-0.90 (m, 6H), 0.70-0.83 (m, 2H).

### Synthesis of 55.8

At 0° C., to a solution of 55.7 (260 mg, 0.66 mmol) and HBr (6.64 mg, 0.033 mmol, 40%) in MeOH (10 mL) was added Br₂ (127 mg, 0.798 mmol). The mixture was stirred at RT for 2 h then poured into saturated NaHCO₃ (20 mL). The suspension was extracted with EtOAc (3×20 mL). The combined organic layer was washed with saturated brine (30 mL), dried over anhydrous Na₂SO₄, filtered and concentrated to give 55.8 (350 mg, crude). ¹H NMR (400 MHz, CDCl₃) δ_{H} 3.86-4.01 (m, 1H), 3.34-3.40 (m, 3H), 3.11-3.20 (m, 2H), 2.56 (dd, J=3.14, 12.67 Hz, 1H), 1.53-2.01 (m, 13H), 1.28-1.52 (m, 8H), 1.04-1.22 (m, 4H), 0.91-1.00 (m, 4H), 0.88 (s, 3H), 0.69-0.84 (m, 2H).

### Synthesis of 55

To a solution of 55.8 (350 mg, 0.745 mmol) in acetone (10 ml) was added K₂CO₃ (205 mg, 1.49 mmol) and 1H-pyrazole-4-carbonitrile (83.2 mg, 0.895 mmol). The mixture was stirred at RT for 2 h then poured into water (20 ml). The suspension was extracted with EtOAc (2×20 ml) and the combined extracts was dried over anhydrous Na₂SO₄, filtered and concentrated. The residue was purified by flash column (0-50% of EtOAc in PE) to give product 55 (180 mg, 50.1%). ¹H NMR (400 MHz, CDCl₃) δ_{H} 7.81 (d, J=5.77 Hz, 2H), 4.92-5.07 (m, 2H), 3.38 (s, 3H), 3.11-3.21 (m, 2H), 2.31 (dd, J=3.14, 12.67 Hz, 1H), 2.04 (s, 1H), 1.81-1.99 (m, 2H), 1.65-1.80 (m, 5H), 1.46-1.59 (m, 5H), 1.16-1.46 (m, 10H), 0.96-1.13 (m, 2H), 0.94 (s, 3H), 0.88 (s, 3H), 0.72-0.86 (m, 2H). LC-ELSD/MS purity>99%, MS ESI calcd. for C₂₉H₄₃N₃O₃Na [M+Na]⁺504.3, found 504.3.

### Example 56: Synthesis of 1-(2-((1S,4aS,4bR,6aS,9S,11aS,11bS,13aS)-9-hydroxy-9-(methoxymethyl)-11a,13a-dimethyloctadecahydro-1H-cyclohepta[a]phenanthren-1-yl)-2-oxoethyl)-1H-pyrazole-4-carbonitrile (56)

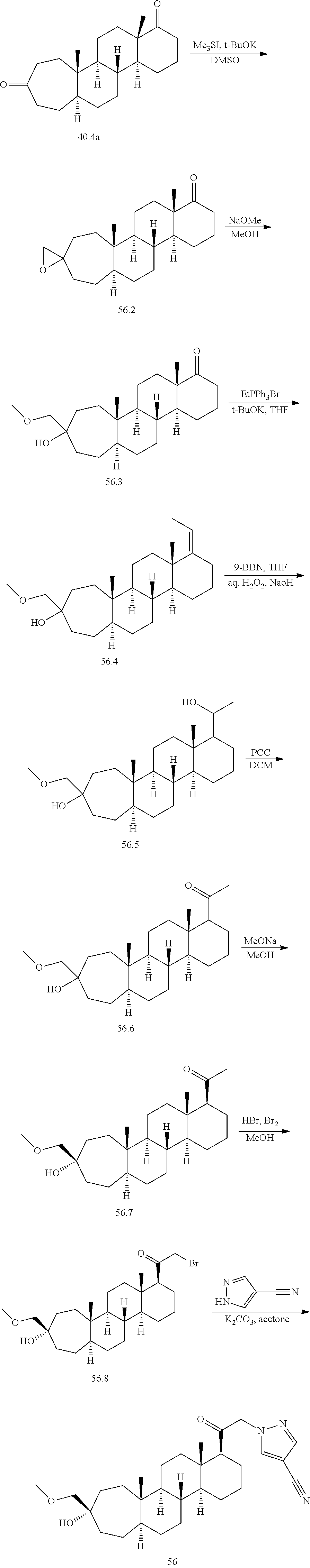

### Synthesis of 56.2

To a suspension of Me₃SI (2.28 g, 10.4 mmol) in DMSO (40 mL) was added t-BuOK (1.16 g, 10.4 mmol). The reaction mixture was warmed to 60° C. and stirred for 1 h. A solution of 40.4a (3.00 g, 9.5 mmol) in DMSO (20 mL) was added and the reaction was stirred at 60° C. for another 16 h. The mixture was cooled, poured into water (100 mL) and extracted with EtOAc (2×50 mL). The combined organic phase was washed with brine (2×100 mL), dried over anhydrous Na₂SO₄, filtered and concentrated. The residue was purified by flash column (0-20% of EtOAc in PE) to give 56.2 (1.83 g, 52.9%). ¹H NMR (400 MHz, CDCl₃) δ_{H} 2.80 (d, J=4.4 Hz, 1H) 2.61 (s, 5H) 1.66-1.89 (m, 8H) 1.31-1.54 (m, 7H) 0.74-1.16 (m, 13H).

### Synthesis of 56.3

Na (1.27 g, 55.3 mmol) was added into MeOH (30 mL) in several small portions and the mixture was stirred at RT for 2 h. A solution of 56.2 (1.83 g, 5.5 mmol) in THF (10 mL) was added and the mixture was warmed to 60° C. and stirred for 6 h. The reaction was cooled and poured into saturated NH₄Cl (100 mL). The suspension was extracted with EtOAc (3×50 mL) and the combined organic phase was washed with saturated brine (100 mL), dried over anhydrous Na₂SO₄, filtered and concentrated. The residue was purified by flash column (0-25% of EtOAc in PE) to give 56.3 (1.35 g, 67.3%). ¹H NMR (400 MHz, CDCl₃) δ_{H} 3.42-3.48 (m, 1H) 3.35-3.38 (m, 3H) 3.12-3.22 (m, 2H) 2.06-2.66 (m, 4H) 1.67-1.84 (m, 5H) 1.30-1.57 (m, 9H) 0.62-1.21 (m, 14H).

### Synthesis of 56.4

To a solution of EtPPh₃Br (8.27 g, 22.3 mmol) in THE (20 mL) was added t-BuOK (2.50 g, 22.3 mmol). The resulting mixture was warmed to 60° C. and stirred for 30 min. 56.3 (1.35 g, 3.7 mmol) was added in portions and the reaction mixture was stirred at 60° C. for 16 h to give a yellow suspension. The reaction was cooled and quenched with 10% NH₄Cl aqueous (50 mL). The layers were separated, and the aqueous layer was extracted with EtOAc (2×50 mL). The combined organic phase was washed with saturated brine (100 mL), dried over anhydrous Na₂SO₄, filtered and concentrated. The residue was triturated with MeOH/H₂O (70 mL/70 ml) at RT to give crude product, which was further purified by flash column (0˜15% of EtOAc in PE) to give 56.4 (810 mg, 59.5%). ¹H NMR (400 MHz, CDCl3) δ_{H} 5.04-5.22 (m, 1H) 3.38 (s, 3H) 3.10-3.25 (m, 2H) 2.49 (m, 1H) 2.09-2.37 (m, 2H) 1.60-2.00 (m, 11H) 1.28-1.57 (m, 7H) 0.67-1.23 (m, 15H).

### Synthesis of 56.5

To a solution of 56.4 (810 mg, 2.2 mmol) in THE (10 mL) was added BH₃-Me₂S (2.16 mL, 10M, 21.6 mmol). The mixture was stirred at RT for 16 h then quenched with EtOH (6.29 mL, 108 mmol). The solution was cooled 0° C. and NaOH (21.6 mL, 5.0M, 108 mmol) was added, followed by H₂O₂(30% in water, 10.7 mL, 108 mmol). The reaction was then heated to 80° C. and stirred for 1 h. After cooling, the mixture was poured into water (50 mL) and extracted with EtOAc (2×30 mL). The combined organic layer was washed with saturated Na₂S₂O₃ (100 mL), brine (100 mL), dried over anhydrous Na₂SO₄, filtered and concentrated to give 56.5 (950 mg, crude). ¹H NMR (400 MHz, CDCl₃) δ_{H}3.37 (s, 3H) 3.12-3.21 (m, 2H) 1.67-1.93 (m, 9H) 1.29-1.63 (m, 11H) 1.03-1.16 (m, 6H) 0.63-0.97 (m, 13H).

### Synthesis of 56.6

At 0° C., to a solution of 56.5 (800 mg, 2.0 mmol) in DCM (10 mL) was added silica gel (655 mg) and PCC (648 mg, 3.0 mmol). The mixture was stirred at 0° C. to RT for 1 h, then PE (5 mL) was added. The mixture was filtered through a pad of silica gel and the filter cake was washed with DCM (2×20 mL). The filtrate was concentrated, and the residue was purified by flash column (0˜-10% of EtOAc in PE) to give 56.6 (670 mg, 84%). ¹H NMR (400 MHz, CDCl₃) δ_{H} 3.38 (s, 3H) 3.11-3.23 (m, 2H) 2.22-2.51 (m, 1H) 2.10-2.14 (m, 3H) 1.59-1.92 (m, 10H) 1.25-1.55 (m, 8H) 0.57-1.21 (m, 15H) Synthesis of 56.7

At 0° C., to a solution of 56.6 (670 mg, 1.71 mmol) in MeOH (45 mL) was added MeONa (1.84 g, 34.2 mmol), and the reaction was heated to 80° C. and stirred for 48 h. After cooling, the reaction mixture was poured into saturated NH₄Cl (100 mL). The resultant suspension was extracted with EtOAc (2×50 mL) and the combined organic phase was washed with saturated brine (100 mL), dried over anhydrous Na₂SO₄, filtered and concentrated. The residue was purified by flash column (0˜20% of EtOAc in PE) to give 56.7 (67.0 mg, 10%).

56.7: ¹H NMR (400 MHz, CDCl₃)^{6H} 3.37 (s, 3H) 3.11-3.18 (m, 2H) 2.15-2.30 (m, 2H) 2.10-2.14 (m, 3H) 1.67-1.91 (m, 5H) 1.52-1.62 (m, 4H) 0.92-1.50 (m, 14H) 0.90 (s, 3H) 0.73-0.83 (m, 3H) 0.70 (s, 3H).

### Synthesis of 56.8

To a solution of 56.7 (47.0 mg, 0.1 mmol) in methanol (2 mL) was added HBr (40%, 4.80 mg, 0.02 mmol). Br₂ (21.1 mg, 0.1 mmol) was added dropwise and the dark solution was stirred at RT for 2 h. NaHCO₃ (30 mL) was added and the suspension was extracted with EtOAc (2×20 mL). The combined organic phase was washed with saturated brine (2×50 mL), dried over anhydrous Na₂SO₄, filtered and concentrated to give 56.8 (50 mg, crude). ¹H NMR (400 MHz, CDCl₃)^{6H} 3.87-4.00 (m, 2H) 3.38 (s, 3H) 3.12-3.19 (m, 2H) 2.50-2.61 (m, 1H) 1.62-1.91 (m, 8H) 1.27-1.52 (m, 9H) 0.95-1.19 (m, 5H) 0.69-0.93 (m, 11H).

### Synthesis of 56

To a solution of 56.8 (50.0 mg, 0.1 mmol) in acetone (2 ml) was added 4-cyanopyrazole (9.90 mg, 0.1 mmol) and K₂CO₃ (29.3 mg, 0.2 mmol). The mixture was stirred at RT for 2 h then poured to water (20 ml). The resultant suspension was extracted with EtOAc (2×20 mL) and the combined organic phase was washed with saturated brine (2×50 mL), dried over anhydrous Na₂SO₄, filtered, concentrated. The residue was purified by CombiFlash (0-40% of EtOAc in PE) to give 56 (30.0 mg, 58%). ¹H NMR (400 MHz, CDCl₃) δ_{H} 7.82 (s, 1H) 7.81 (s, 1H) 4.93-5.06 (m, 2H) 3.38 (s, 3H) 3.12-3.20 (m, 2H) 2.31 (dd, J=12.8, 3.2 Hz, 1H) 2.19 (s, 1H) 1.58-1.91 (m, 9H) 0.96-1.49 (m, 13H) 0.93 (s, 3H) 0.73-0.89 (m, 4H) 0.71 (s, 3H). LC-ELSD/MS purity 99%, MS ESI calcd. for C₂₉H₄₃N₃O₃ [M+H]⁺ 482.3, found 482.3.

### Example 57 & 58: Synthesis of 1-((1S,4aS,4bS,9S,11aR,11bS,13aS)-9-hydroxy-9,11a,13a-trimethyl-2,3,4,4a,4b,5,7,8,9,10,11,11a,11b,12,13,13a-hexadecahydro-1H-cyclohepta[a]phenanthren-1-yl)ethanone (57) & Synthesis of 1-(2-((1S,4aS,4bS,9S,11aR,11bS,13aS)-9-hydroxy-9,11a,13a-trimethyl-2,3,4,4a,4b,5,7,8,9,10,11,11a,11b,12,13,13a-hexadecahydro-1H-cyclohepta[a]phenanthren-1-yl)-2-oxoethyl)-1H-pyrazole-4-carbonitrile (58)

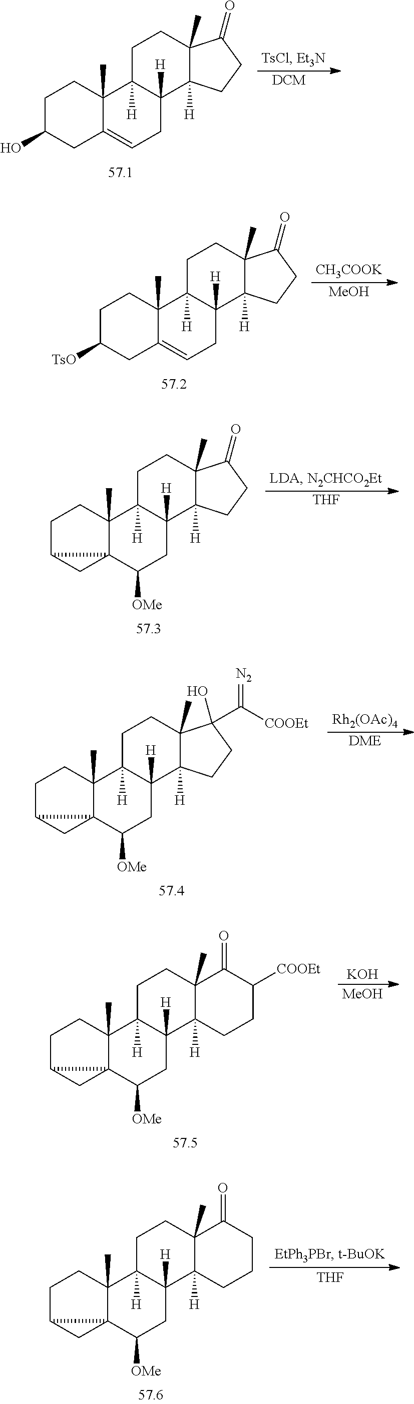

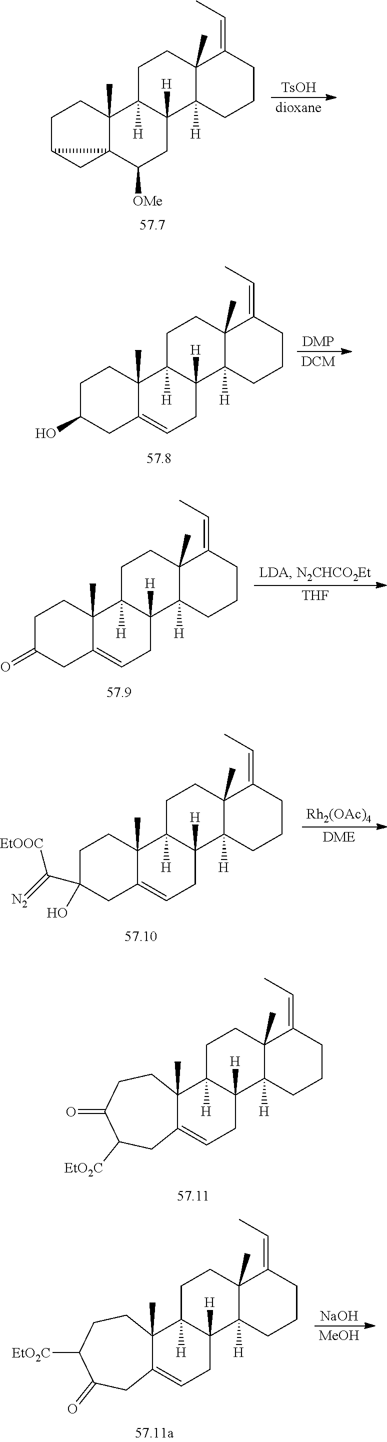

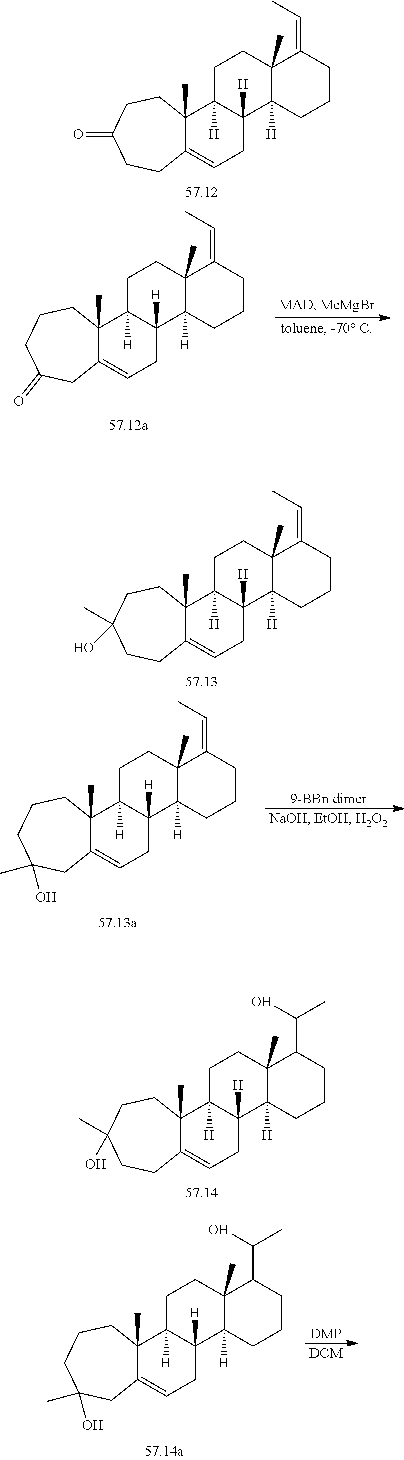

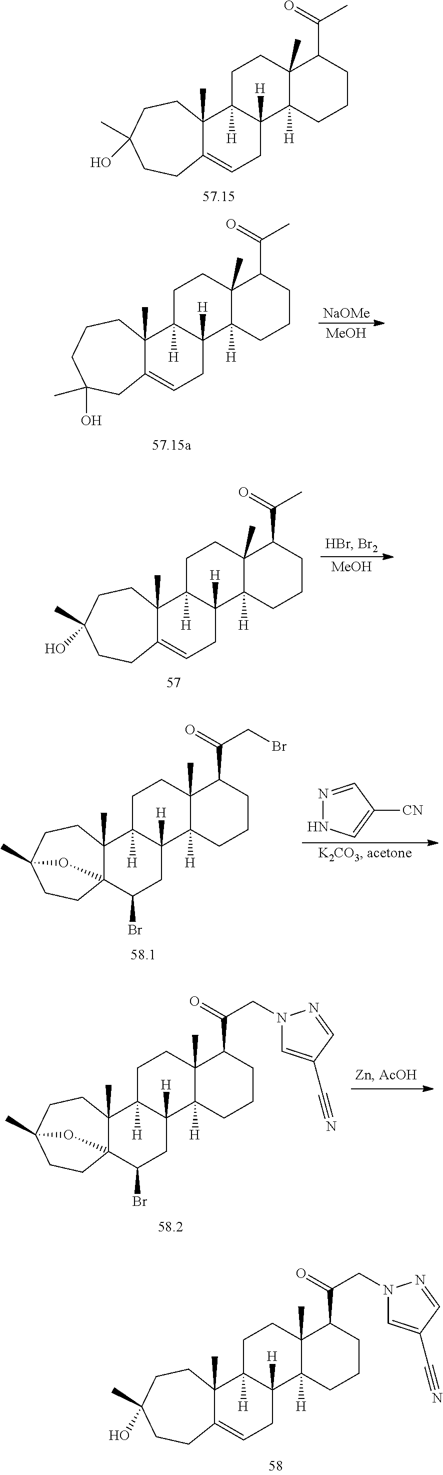

### Synthesis of 57.2

A solution of 57.1 (20.0 g, 69.3 mmol), 1-methyl-1H-imidazole (8.44 g, 103 mmol), TsCl (39.4 g, 207 mmol) and Et₃N (41.9 g, 415 mmol) in DCM (200 mL) was stirred at RT for 1 h. The mixture was poured into HCl (300 mL, 0.5 M), then extracted with DCM (2×100 mL). The combined organic layer was washed with water (500 mL), saturated brine (500 mL), dried over anhydrous Na₂SO₄, filtered and concentrated to give 57.2 (35.0 g, crude). ¹H NMR (400 MHz, CDCl₃) δ_{H} 7.80 (d, J=8.4 Hz, 2H), 7.33 (d, J=8.0 Hz, 2H), 5.35 (d, J=5.2 Hz, 1H), 4.40-4.25 (m, 1H), 2.50-1.59 (m, 18H), 1.50-1.20 (m, 4H), 0.99 (s, 3H), 0.87 (s, 3H).

### Synthesis of 57.3

A solution of 57.2 (35.0 g, crude) and CH₃COOK (42.5 g, 434 mmol) in MeOH (300 mL) was stirred at 80° C. for 16 h. The mixture was concentrated and water (300 mL) and EtOAc (150 mL) was added. The layers were separated, and the aqueous layer was extracted with EtOAc (2×100 mL). The combined organic layer was washed with saturated brine (300 mL), dried over anhydrous Na₂SO₄, filtered and concentrated. The residue was purified by flash column (0-8% of EtOAc in PE) to give 57.3 (20.0 g, 84%). ¹H NMR (400 MHz, CDCl₃) δ_{H} 3.35 (s, 3H), 2.82 (t, J=2.4 Hz, 1H), 2.55-2.45 (m, 1H), 2.20-1.60 (m, 6H), 1.52-1.08 (m, 8H), 1.04 (s, 3H), 0.91 (s, 3H), 0.90-0.80 (m, 3H), 0.75-0.70 (m, 1H), 0.51-0.42 (m, 1H).

### Synthesis of 57.4

A fresh prepared LDA (330 mmol, in 200 mL THF) solution was cooled to −70° C. then added to a stirred and cooled (−70° C.) solution of 57.3 (20.0 g, 66.1 mmol) and ethyl diazoacetate (37.6 g, 330 mmol) in THE (350 mL). The mixture was stirred at −70° C. for 2 h and HOAc (19.8 g, 330 mmol) in THE (50 mL) was added. The mixture was allowed to warm to RT and stirred for 16 h. Water (500 mL) was added and the mixture was extracted with EtOAc (3×150 mL). The combined organic layers were washed with brine (500 mL), dried over anhydrous Na₂SO₄, filtered and concentrated. The residue was purified by flash column (0˜10% of EtOAc in PE) to give 57.4 (27.4 g, 99%). ¹H NMR (400 MHz, CDCl₃) δ_{H} 4.68 (s, 1H), 4.35-4.20 (m, 2H), 3.35 (s, 3H), 2.78 (t, J=2.0 Hz, 1H), 2.25-2.15 (m, 1H), 2.00-1.60 (m, 5H), 1.52-1.25 (m, 9H), 1.10-1.05 (m, 2H), 1.03 (s, 3H), 0.97 (s, 3H), 0.95-0.75 (m, 4H), 0.67-0.60 (m, 1H), 0.48-0.40 (m, 1H).

### Synthesis of 57.5

A solution of 57.4 (27.4 g, 65.7 mmol) and Rh₂(OAc)₄ (290 mg, 0.66 mmol) in DME (300 mL) was stirred at RT for 2 h. The mixture was concentrated and water (200 mL) and EtOAc (200 mL) was added. The layers were and the aqueous layer was extracted with EtOAc (2×100 mL). The combined organic layer was washed with saturated brine (200 mL), dried over anhydrous Na₂SO₄, filtered and concentrated to give 57.5 (23.5 g, crude).

### Synthesis of 57.6

A solution of 57.5 (23.5 g, 60.4 mmol) and KOH (20.3 g, 362 mmol) in MeOH (300 mL) was stirred at 70° C. for 2 h. The mixture was concentrated, water (200 mL) and EtOAc (200 mL) were added. The layers were separated, and the aqueous layer was extracted with EtOAc (2×150 mL). The combined organic layer was washed with saturated brine (300 mL), dried over anhydrous Na₂SO₄, filtered and concentrated to give 57.6 (17.0 g, crude). ¹H NMR (400 MHz, CDCl₃) δ_{H} 3.35 (s, 3H), 2.83 (t, J=2.8 Hz, 1H), 2.75-2.60 (m, 1H), 2.30-2.10 (m, 3H), 1.95-1.70 (m, 5H), 1.55-1.20 (m, 7H), 1.14 (s, 3H), 1.10-1.05 (m, 1H), 1.02 (s, 3H), 0.95-0.80 (m, 3H), 0.75-0.70 (m, 1H), 0.55-0.45 (m, 1H).

### Synthesis of 57.7

To a solution of EtPh₃PBr (119 g, 322 mmol) in THF (300 mL) was added t-BuOK (36.1 g, 322 mmol). The mixture was warmed to 50° C. and stirred for 1h. A solution of 57.6 (17.0 g, 53.7 mmol) in THF (200 mL) was added while keeping the internal temperature below 60° C. The mixture was stirred at 60° C. for 16 h, then cooled to RT. Saturated NH₄Cl (500 mL) was added and the resultant suspension was extracted with EtOAc (3×200 mL). The combined organic layer was washed with saturated brine (500 mL), dried over anhydrous Na₂SO₄, filtered and concentrated. The residue was triturated with MeOH/water (800 mL/800 mL) to give 57.7 (18.6 g, impure). ¹H NMR (400 MHz, CDCl₃) δ_{H} 5.25-5.10 (m, 1H), 3.35 (s, 3H), 2.80 (t, J=2.4 Hz, 1H), 2.55-2.10 (m, 3H), 2.00-1.60 (m, 8H), 1.52-1.30 (m, 5H), 1.22-0.80 (m, 13H), 0.70-0.65 (m, 1H), 0.50-0.40 (m, 1H).

### Synthesis of 57.8

To a solution of 57.7 (18.6 g, 56.6 mmol) in 1,4-dioxane (100 mL) was added a solution of TsOH (1.07 g, 5.66 mmol) in water (30 mL). The mixture was heated to 75° C. and stirred for 16 h. The mixture was cooled, poured into saturated NaHCO₃ (200 mL) and extracted with DCM (3×100 mL). The combined organic layer was washed with saturated brine (300 mL), dried over anhydrous Na₂SO₄, filtered and concentrated. The residue was purified by flash column (0-20% of EtOAc in PE) to give 57.8 (16.0 g, 90%). ¹H NMR (400 MHz, CDCl₃) δ_{H} 5.35-5.33 (m, 1H), 5.25-5.10 (m, 1H), 3.60-3.45 (m, 1H), 2.55-2.45 (m, 1H), 2.40-2.10 (m, 4H), 2.00-1.59 (m, 9H), 1.52-1.10 (m, 8H), 1.08-0.80 (m, 9H).

### Synthesis of 57.9

A solution of 57.8 (5.00 g, 15.8 mmol) and Dess Martin Reagent (13.4 g, 31.6 mmol) in DCM (100 mL) was stirred at 40° C. for 1 h. The mixture was cooled, poured into saturated NaHCO₃ (300 mL) and extracted with DCM (3×100 mL). The combined organic layer was washed with saturated Na₂S₂O₃ (2×200 mL), saturated brine (200 mL), dried over anhydrous Na₂SO₄, filtered and concentrated to give 57.9 (4.50 g, crude). ¹H NMR (400 MHz, CDCl₃) δ_{H} 5.33 (dd, J=8.8 Hz, 11.2 Hz, 1H), 5.25-5.15 (m, 1H), 3.26 (dd, J=2.8 Hz, 16.4 Hz, 1H), 2.82 (dd, J=2.0 Hz, 16.4 Hz, 1H), 2.60-2.10 (m, 6H), 2.00-1.58 (m, 10H), 1.52-1.30 (m, 3H), 1.28-0.80 (m, 9H).

### Synthesis of 57.10

At −70° C., to a solution of 57.9 (4.50 g, 14.4 mmol) and ethyl diazoacetate (8.21 g, 72.0 mmol) in THF (120 mL) was added LDA (36.0 mL, 2.0 M, 72.0 mmol). The mixture was stirred at −70° C. for 2 h and HOAc (4.32 g, 72.0 mmol) in THF (30 mL) was added at −70° C. The mixture was allowed to warm to 20° C. and stirred for 16 h. The mixture was poured into water (200 mL). and extracted with EtOAc (3×50 mL). The combined organic layer was washed with saturated brine (100 mL), dried over anhydrous Na₂SO₄, filtered and concentrated. The residue was purified by flash column (0-10% of EtOAc in PE) to give 57.10 (2.70 g, 43.9%). ¹H NMR (400 MHz, CDCl₃)^{6H} 5.41 (t, J=2.8 Hz, 1H), 5.28-5.10 (m, 1H), 4.30-4.20 (m, 2H), 3.12 (s, 1H), 2.70-1.60 (m, 15H), 1.52-1.15 (m, 11H), 1.10-0.80 (m, 7H).

### Synthesis of 57.11 & 57.11a

A solution of 57.10 (2.70 g, 6.32 mmol) and Rh₂(OAc)₄ (55.8 mg, 0.13 mmol) in DME (30 mL) was stirred at RT for 16 h. The mixture was poured into saturated brine (100 mL) and extracted with EtOAc (3×30 mL). The combined organic layer was washed with saturate brine (100 mL), dried over anhydrous Na₂SO₄, filtered and concentrated to give 57.11 & 57.11a (2.00 g, crude).

### Synthesis of 57.12 & 57.12a

A solution of 57.11 (2.00 g, 5.01 mmol, include 57.11a) and NaOH (1.68 g, 30.0 mmol) in MeOH (30 mL) was stirred at 75° C. for 16 h. The mixture was cooled, poured into saturated NH₄Cl (100 mL) solution and the suspension was extracted with DCM (3×30 mL). The combined organic layer was washed with saturated brine (100 mL), dried over anhydrous Na₂SO₄, filtered and concentrated. The residue was purified by flash column (0-5% of EtOAc in PE) to give 57.12 & 57.12a (1.00 g, impure).

### Synthesis of 57.13 & 57.13a

A freshly prepared solution of MAD (25.7 mmol, in 50 mL toluene) was cooled to −70° C. and a solution of 57.12 (2.80 g, 8.57 mmol, include 57.12a) in DCM (40 mL) was added. The mixture was stirred at −70° C. for 1 h, MeMgBr (8.56 mL, 25.7 mmol, 3M) was added. The mixture was stirred at −70° C. for 2 h then poured into citric acid (200 mL, 10% in water) solution. The suspension was extracted with EtOAc (3×50 mL) and the combined organic layer was washed with saturated brine (200 mL), dried over anhydrous Na₂SO₄, filtered and concentrated. The residue was purified by flash column (0-10% of EtOAc in PE) to give 57.13 (1.20 g, impure, include 57.13a).

### Synthesis of 57.14 & 57.14a

To a solution of 57.13 (1.20 g, include 57.13a) in THF (20 mL) was added 9-BBN dimer (3.38 g, 14.0 mmol), and the mixture was stirred at 60° C. for 16 h. After cooling, EtOH (3.86 g, 84.0 mmol) was added. The mixture was stirred for 15 min then cooled 0° C., NaOH (16.8 mL, 5.0M, 84.0 mmol) was added followed by H₂O₂(9.50 g, 84.0 mmol, 30%). The mixture was warmed to 70° C. and stirred for 1 h. After cooling, the mixture was poured into water (100 mL) and extracted with EtOAc (3×50 mL). The combined organic layer was washed with saturated Na₂S₂O₃ (150 mL), saturated brine (150 mL), dried over anhydrous Na₂SO₄, filtered and concentrated. The residue was purified by flash column (0-60% of EtOAc in PE) to give 57.14 (2.00 g, include 57.14a).

### Synthesis of 57.15 & 57.15a

A solution of 57.14 (2.00 g, include 57.14a) and Dess Martin Reagent (4.66 g, 11.0 mmol) in DCM (100 mL) was stirred at 40° C. for 30 min. The mixture was poured into saturated NaHCO₃ (200 mL) and extracted with DCM (3×30 mL). The combined organic layer was washed with Na₂S₂O₃ (2×200 mL), saturated brine (200 mL), dried over anhydrous Na₂SO₄, filtered and concentrated to give 57.15 (1.00 g, crude, include 57.15a).

### Synthesis of 57

A solution of 57.15 (400 mg, include 57.15a) and MeONa (1.19 g, 22.2 mmol) in MeOH (20 mL) was stirred at reflux for 48 h. After cooling, the mixture was poured into saturated NH₄Cl (100 mL) and extracted with DCM (3×30 mL). The combined organic layer was washed with saturated brine (100 mL), dried over anhydrous Na₂SO₄, filtered and concentrated. The residue was purified by flash column (0-15% of EtOAc in PE) to give 57 (300 mg), which was further purified by prep-HPLC (Column: Phenomenex Gemini-NX 150*30 mm*5 μm, Condition: water (0.04% NH₃H₂O+10 mM NH₄HCO₃)-ACN, Begin B: 60, End B: 90, Gradient Time (min): 8, 100% B Hold Time (min): 2) to give 57 (150 mg). SFC purification of the mixture (Column: DAICEL CHIRALPAK AD-H (250 mm*30 mm*5 μm), Condition: 0.1% NH₃H₂O IPA, Begin B: 25, End B: 25) afforded 57 (20.0 mg, 13%).

57: ¹H NMR (400 MHz, CDCl₃) δ_{H} 5.44 (d, J=3.2 Hz, 1H), 2.30 (dd, J=3.2 Hz, 12.4 Hz, 1H), 2.25-2.10 (m, 4H), 2.00-1.59 (m, 9H), 1.52-1.27 (m, 9H), 1.25 (s, 3H), 1.24-0.95 (m, 4H), 0.94 (s, 3H), 0.85 (s, 3H), 0.84-0.78 (m, 1H). LCMS purity>99%, MS ESI calcd. for C₂₄H₃₇O [M−H₂O+H]⁺ 341.3, found 341.3.

### Synthesis of 58.1

At 0° C., to a solution of 57 (20.0 mg, 0.05 mmol) and HBr (1 mg, 40%) in MeOH (5 mL) was added Br₂ (22.3 mg, 0.14 mmol). The mixture was stirred at RT for 2 h and poured into saturated NaHCO₃ (30 mL). The suspension was extracted with EtOAc (2×30 mL) and the combined organic layer was washed with saturated brine (50 mL), dried over anhydrous Na₂SO₄, filtered and concentrated to give 58.1 (30.0 mg, crude). ¹H NMR (400 MHz, CDCl3) δ_{H} 4.15-4.07 (m, 1H), 4.01-3.84 (m, 2H), 2.74-2.53 (m, 1H), 2.29-2.20 (m, 1H), 2.16-2.08 (m, 1H), 1.92-1.83 (m, 3H), 1.72-1.62 (m, 6H), 1.56-1.43 (m, 5H), 1.34-1.22 (m, 8H), 1.10 (s, 3H), 1.04-0.97 (m, 4H), 0.90-0.83 (m, 1H).

### Synthesis of 58.2

To a solution of 58.1 (90.0 mg, 0.17 mmol) in acetone (5 mL) was added K₂CO₃ (48.0 mg, 0.35 mmol) and 1H-pyrazole-4-carbonitrile (19.4 mg, 0.21 mmol), and the mixture was stirred at RT for 2 h. The reaction was quenched with saturated aqueous NH₄Cl solution (50 mL) and extracted with EtOAc (2×30 mL). The combined organic phase was washed with saturated brine (50 mL), dried over anhydrous Na₂SO₄, filtered and concentrated. The residue was purified by silica gel chromatography (0-60% of EtOAc in PE) to give the product 58.2 (40.0 mg, 43.4%). ¹H NMR (400 MHz, CDCl₃) δ_{H} 7.86-7.78 (m, 2H), 5.00 (q, J=18.4 Hz, 2H), 4.15-4.08 (m, 1H), 2.37-2.31 (m, 1H), 2.28-2.22 (m, 1H), 2.16-2.09 (m, 1H), 1.96-1.62 (m, 9H), 1.53-1.47 (m, 3H), 1.43-1.28 (m, 4H), 1.27-1.15 (m, 7H), 1.12 (s, 3H), 1.06-1.03 (m, 1H), 1.00 (s, 3H).

### Synthesis of 58

To a solution of 58.2 (30.0 mg, 0.06 mmol) in HOAc (10 mL) and water (0.5 mL) was added Zinc (738 mg, 11.3 mmol). The mixture was stirred at RT for 1 h then poured into saturated aqueous NH₄Cl (50 mL) solution. The resultant suspension was extracted with EtOAc (3×20 mL) and the combined organic layer was washed with saturated brine (50 mL), dried over anhydrous Na₂SO₄, filtered and concentrated. The residue was purified by CombiFlash (0-20% of EtOAc in PE) to give 58 (18.5 mg, 60.1%). ¹H NMR (400 MHz, CDCl₃) δ_{H} 7.87-7.79 (m, 2H), 5.49-5.41 (m, 1H), 5.09-4.91 (m, 2H), 2.38-2.29 (m, 1H), 2.23-2.12 (m, 1H), 2.01-1.61 (m, 9H), 1.51-1.34 (m, 8H), 1.29-1.18 (m, 7H), 1.11-1.00 (m, 1H), 0.97 (s, 3H), 0.91-0.83 (m, 4H). LCMS purity>99%, MS ESI calcd. for C₂₅H₃₈N₃O [M+H-H₂O]⁺ 432.3, found 432.3.

### Example 59 & 60 & 61 & 62 & 63 & 64 & 65: Synthesis of 1-((1S,5aS,5bR,7aR,9R,12aS,12bS,14aS)-9-hydroxy-9,12a,14a-trimethylicosahydrodicyclohepta[a,f]naphthalen-1-yl)ethanone (59) & Synthesis of 1-(2-((1S,5aS,5bR,7aR,9R,12aS,12bS,14aS)-9-hydroxy-9,12a,14a-trimethylicosahydrodicyclohepta[a,f|naphthalen-1-yl)-2-oxoethyl)-1H-pyrazole-4-carbonitrile (60) & Synthesis of 1-(2-((1R,5aS,5bR,7aR,9R,12aS,12bS,14aS)-9-hydroxy-9,12a,14a-trimethylicosahydrodicyclohepta[a,f]naphthalen-1-yl)-2-oxoethyl)-1H-pyrazole-4-carbonitrile (61) & Synthesis of 1-((1S,5aS,5bR,7aR,9R,12aS,12bS,14aS)-9-hydroxy-9,12a,14a-trimethylicosahydrodicyclohepta[a,f]naphthalen-1-yl)-2-(5-methyl-2H-tetrazol-2-yl)ethanone (62) & Synthesis of 1-((1S,5aS,5bR,7aR,9R,12aS,12bS,14aS)-9-hydroxy-9,12a,14a-trimethylicosahydrodicyclohepta[a,f]naphthalen-1-yl)-2-(5-methyl-1H-tetrazol-1-yl)ethanone (63) & Synthesis of 1-((1R,5aS,5bR,7aR,9R,12aS,12bS,14aS)-9-hydroxy-9,12a,14a-trimethylicosahydrodicyclohepta[a,flnaphthalen-1-yl)-2-(5-methyl-2H-tetrazol-2-yl)ethanone (64) & Synthesis of 1-((1R,5aS,5bR,7aR,9R,12aS,12bS,14aS)-9-hydroxy-9,12a,14a-trimethylicosahydrodicyclohepta[a,f]naphthalen-1-yl)-2-(5-methyl-1H-tetrazol-1-yl)ethanone (65)

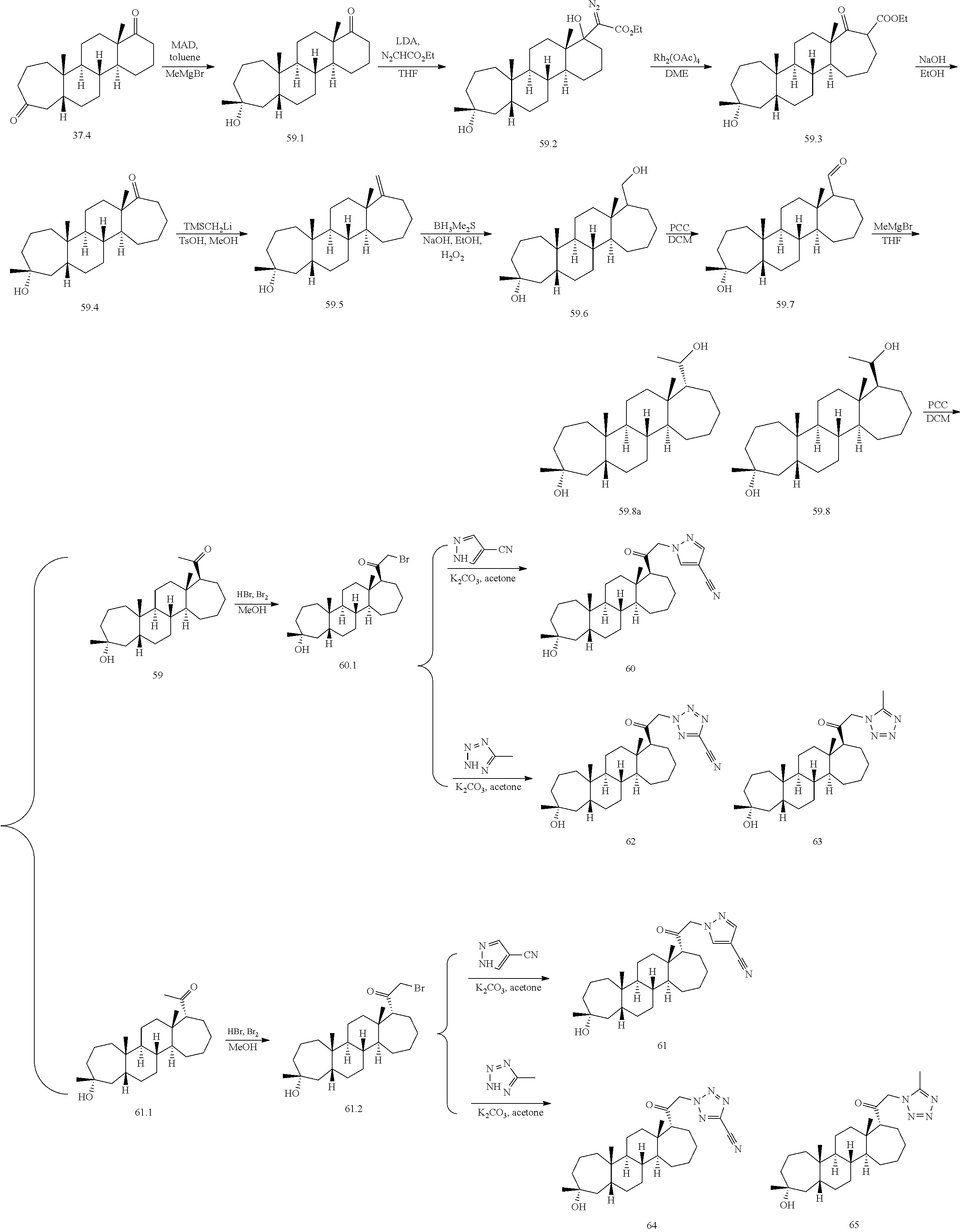

### Synthesis of 59.1

A freshly prepared MAD (18.9 mmol) solution was cooled to −70° C. and 37.4 (2 g, 6.31 mmol) in DCM (20 mL) was added dropwise. After stirring at −70° C. for 1 h, MeMgBr (6.30 mL, 3 M in ethyl ether, 18.9 mmol) was added dropwise and the resulting solution was stirred at −70° C. for 4 h. The reaction mixture was slowly poured into saturated aqueous citric acid (200 mL) while keep the internal temperature below 10° C. The resultant suspension was extracted with EtOAc (2×100 mL) and the combined organic layer was dried over Na₂SO₄, filtered and concentrated. The residue was purified by flash column (0-30% of EtOAc in PE) to give product 59.1 (870 mg, 41.7%). ¹H NMR (400 MHz, CDCl₃) δ_{H} 2.67-2.56 (m, 1H), 2.23-2.15 (m, 1H), 2.09-1.95 (m, 2H), 1.87-1.69 (m, 5H), 1.68-1.58 (m, 2H), 1.53-1.29 (m, 8H), 1.28-1.16 (m, 7H), 1.14-0.93 (m, 6H), 0.89 (s, 3H), 0.87-0.76 (m, 1H). LC-ELSD/MS: purity>99%, MS ESI calcd. for C₂₂H₃₂ [M-2H₂O]⁺ 297.2, found 297.2.

### Synthesis of 59.2

At −70° C., a cooled (−70° C.) LDA solution (108 mmol) was added to a stirred solution of 59.1 (6.00 g, 18.0 mmol) and ethyl diazoacetate (12.3 g, 108 mmol) in THF (180 mL). The mixture was stirred at −70° C. for 2 h, then acetic acid (6.47 g, 108 mmol) in THE (20 mL) was added. The mixture was allowed to warm to RT and stirred for 16 h. Water (300 mL) and PE (300 mL) were added, and the layers were separated. The aqueous layer was extracted with EtOAc (200 mL) and the combined organic layers were washed with saturated brine (500 mL), dried over anhydrous Na₂SO₄, filtered and concentrated. The residue was purified by flash column (0˜35% of EtOAc in PE) to give product 59.2 (5.00 g, 83.6%). ¹H NMR (400 MHz, CDCl₃) δ 4.28-4.18 (m, 2H), 2.04-1.67 (m, 8H), 1.65-1.41 (m, 9H), 1.37-1.22 (m, 11H), 1.20-0.94 (m, 6H), 0.88-0.82 (m, 6H) Synthesis of 59.3

To a solution of 59.2 (5.00 g, 11.1 mmol) in DME (100 mL) was added Rh₂(OAc)₄ (98.1 mg, 0.22 mmol), and the mixture was stirred at 50° C. for 16 h. The reaction mixture was concentrated to give the product 59.3 (5.10 g, crude), which was used to the next step without further purification. ¹H NMR (400 MHz, CDCl₃) δ 4.57-3.93 (m, 3H), 2.27-1.96 (m, 2H), 1.88-1.54 (m, 10H), 1.51-1.32 (m, 7H), 1.31-1.16 (m, 11H), 1.15-1.01 (m, 5H), 0.94-0.75 (m, 4H).

### Synthesis of 59.4

To a solution of 59.3 (5.00 g, 11.9 mmol) in EtOH (100 mL) was added H₂O (30 mL) and NaOH (7.11 g, 178 mmol). The mixture was stirred at 80° C. for 16 h then cooled and concentrated. H₂O (200 mL) and EtOAc (10 mL) were added and the layers are separated. The aqueous layer was extracted with EtOAc (2×150 mL) and the combined organic phase was washed with saturated brine (200 mL), dried over anhydrous Na₂SO₄, filtered and concentrated to give the product (2.50 g, crude). 50 mg crude 59.4 was purified by silica gel chromatography (0-30% of EtOAc in PE) to give the pure product 59.4 (23.2 mg). ¹H NMR (400 MHz, CDCl₃) δ 3.08-2.96 (m, 1H), 2.32-2.21 (m, 1H), 2.12-2.00 (m, 1H), 1.88-1.59 (m, 8H), 1.56-1.35 (m, 9H), 1.34-1.22 (m, 6H), 1.21-1.07 (m, 5H), 1.04 (s, 3H), 0.93-0.82 (m, 4H). LC-ELSD/MS: purity>99%, MS ESI calcd. for C₂₃H₃₇O [M+H-H₂O]⁺ 329.3, found 329.3.

### Synthesis of 59.5

At −40° C., to a solution of TMSCH₂Li (118 mL, 66.3 mmol, 0.56 M) in THF (20 mL) was added a solution of 59.4 (2.30 g, 6.63 mmol) in THE (20 mL). The reaction was warmed to RT and stirred for 16 h. The mixture was poured into saturated NH₄C1 (100 mL) and extracted with EtOAc (2×60 mL). The combined organic layer was washed with saturated brine (100 mL), dried over anhydrous Na₂SO₄, filtered and concentrated. The residue was dissolved in MeOH (50 mL), and TsOH (50 mg) was added. The mixture was stirred for 10 min then poured into saturated NaHCO₃ (100 mL). The suspension was extracted with EtOAc (3×50 mL) and the combined organic layer was washed with saturated brine (100 mL), dried over anhydrous Na₂SO₄, filtered and concentrated. The residue was purified by flash column (0-25% of EtOAc in PE) to give 59.5 (2.00 g, 87.5%). ¹H NMR (400 MHz, CDCl₃)⁶H 4.80 (d, J=1.6 Hz, 1H), 4.73 (s, 1H), 2.47-2.34 (m, 1H), 2.24-2.13 (m, 1H), 2.11-2.04 (m, 1H), 1.87-1.64 (m, 6H), 1.49-1.30 (m, 9H), 1.28-1.11 (m, 11H), 1.08-0.96 (m, 5H), 0.90-0.76 (m, 4H).

### Synthesis of 59.6

To a solution of 59.5 (2.00 g, 5.80 mmol) in THE (30 mL) was added BH₃-Me₂S (2.88 mL, 28.9 mmol, 10 M). The mixture was stirred at 45° C. for 1 h then cooled to 0° C. Ethanol (5.29 g, 115 mmol) was added, followed by NaOH aqueous (23.0 mL, 5.0 M, 115 mmol). H₂O₂(11.5 mL, 10 M, 115 mmol) was added dropwise while keeping the internal temperature below 15° C. The reaction mixture was then heated to 70° C. and stirred for 1 h. The mixture was cooled and poured into water (100 mL) and extracted with EtOAc (2×50 mL). The combined organic layer was washed with saturated aq. Na₂SO₃ (100 mL), brine (100 mL), dried over anhydrous Na₂SO₄, filtered and concentrated to give 59.6 (2.10 g, crude). ¹H NMR (400 MHz, CDCl₃) δ_{H} 3.90-3.70 (m, 1H), 3.35-3.22 (m, 1H), 2.10-2.04 (m, 1H), 1.99-1.64 (m, 7H), 1.55-1.32 (m, 10H), 1.29-1.01 (m, 14H), 0.94-0.69 (m, 8H).

### Synthesis of 59.7

To a solution of 59.6 (2.10 g, crude) in DCM (30 mL) was added silica gel (3.00 g) and PCC (2.47 g, 11.5 mmol), and the mixture was stirred at RT for 1 h. The suspension was filtered, and the filter cake was washed with DCM (3×50 mL). The filtrate was concentrated to give 59.7 (2.10 g, crude), which was used directly to the next step.

### Synthesis of 59.8 & 59.8a

At 0° C., to a solution of 59.7 (2.10 g, crude) in THE (30 mL) was added MeMgBr (9.70 mL, 3M in ethyl ether, 29.1 mmol). The reaction mixture was allowed to warm to RT and stirred for 1 h. The resultant black suspension was poured into saturated NH₄Cl (150 mL) then extracted with EtOAc (3×100 mL). The combined organic layer was washed with saturated brine (100 mL), dried over anhydrous Na₂SO₄, filtered and concentrated. The residue was purified by flash column (0-40% of EtOAc in PE) to give 59.8 (450 mg, 20.4%) and 59.8a (450 mg, 20.4%).

59.8: ¹H NMR (400 MHz, CDCl₃) δ 4.32-4.19 (m, 1H), 2.07-1.70 (m, 8H), 1.45-1.34 (m, 9H), 1.27-1.10 (m, 12H), 1.05-0.96 (m, 3H), 0.93-0.71 (m, 11H).

59.8a: ¹H NMR (400 MHz, CDCl₃) δ 4.16-4.02 (m, 1H), 2.11-1.92 (m, 2H), 1.85-1.66 (m, 5H), 1.56-1.37 (m, 8H), 1.35-1.23 (m, 8H), 1.22-1.07 (m, 10H), 1.04-0.97 (m, 2H), 0.92-0.72 (m, 8H).

### Synthesis of 59

To a solution of 59.8 (450 mg, 1.19 mmol) in DCM (20 mL) was added silica gel (1.00 g) and PCC (511 mg, 2.38 mmol). The reaction mixture was stirred at RT for 1 h to give a brown suspension. The mixture was filtered, and the filter cake was washed with DCM (3×20 mL). The filtrate was concentrated, and the residue was purified by flash column (0-20% of EtOAc in PE) to give product 59 (410 mg, impure). 40.0 mg crude 59 was purified by silica gel chromatography (0-25% of EtOAc in PE) to give the pure product 59 (20.6 mg). ¹H NMR (400 MHz, CDCl₃) δ 2.42-2.31 (m, 1H), 2.13 (s, 3H), 2.07-1.99 (m, 1H), 1.87-1.61 (m, 8H), 1.55-1.31 (m, 9H), 1.28-1.12 (m, 10H), 1.07-0.96 (m, 5H), 0.87-0.75 (m, 5H). LC-ELSD/MS: purity>99%, MS ESI calcd. for C₂₅H₄₁O [M−H₂O+H]⁻ 357.3, found 357.3.

### Synthesis of 60.1

At 0° C., to a solution of 59 (70.0 mg, 0.18 mmol) and HBr (3 mg, 0.01 mmol, 40%) in MeOH (5 mL) was added Br₂ (35.8 mg, 0.22 mmol). The mixture was allowed to warm to RT and stirred for 2 h. The reaction was poured into saturated NaHCO₃ (20 mL) then extracted with EtOAc (3×20 mL). The combined organic layer was washed with saturated brine (30 mL), dried over anhydrous Na₂SO₄, filtered and concentrated to give 60.1 (90.0 mg, crude), which was used to next step directly.

### Synthesis of 60

To a solution of 60.1 (70.0 mg, 0.15 mmol) in acetone (5 mL) was added K₂CO₃ (42.5 mg, 0.31 mmol) and 1H-pyrazole-4-carbonitrile (17.2 mg, 0.18 mmol). The reaction mixture was stirred at RT for 2 h then quenched with saturated aqueous NH₄C solution (30 mL). The suspension was extracted with EtOAc (2×30 mL) and the combined organic phase was washed with saturated brine (50 mL), dried over anhydrous Na₂SO₄, filtered and concentrated. The residue was purified by silica gel chromatography (0-70% of EtOAc in PE) to give product 60 (16.9 mg, 23.6%). ¹H NMR (400 MHz, CDCl₃) δ 7.84-7.79 (m, 2H), 4.99 (s, 2H), 2.42-2.33 (m, 1H), 2.09-1.98 (m, 1H), 1.87-1.63 (m, 8H), 1.56-1.32 (m, 8H), 1.30-1.09 (m, 11H), 1.08-0.97 (m, 5H), 0.89-0.73 (m, 5H). LC-ELSD/MS: purity>99%, MS ESI calcd. for C₂₉H₄₂N₃O [M−H₂O+H]⁺448.3, found 448.3.

### Synthesis of 61.1

To a solution of 59.8a (450 mg, 1.19 mmol) in DCM (20 mL) was added silica gel (1.00 g) and PCC (511 mg, 2.38 mmol). The mixture was stirred at RT for 2 h to give a brown suspension. The mixture was filtered, and the filter cake was washed with DCM (3×20 mL). The filtrate was concentrated, and the residue was purified by flash column (0-20% of EtOAc in PE) to give product 61.1 (400 mg, impure). ¹H NMR (400 MHz, CDCl₃) δ 2.43 (d, J=10.0 Hz, 1H), 2.15-2.02 (m, 4H), 1.91-1.59 (m, 9H), 1.52-1.30 (m, 7H), 1.28-1.10 (m, 13H), 1.07-0.97 (m, 4H), 0.89-0.77 (m, 4H).

### Synthesis of 61.2

At 0° C., to a solution of 61.1 (70.0 mg, 0.18 mmol) and HBr (3.00 mg, 0.01 mmol, 40%) in MeOH (5 mL) was added Br₂ (35.8 mg, 0.22 mmol). The mixture was warmed 25° C. and stirred for 2 h. The mixture was poured into saturated NaHCO₃ (20 mL) then extracted with EtOAc (3×20 mL). The combined organic layer was washed with saturated brine (30 mL), dried over anhydrous Na₂SO₄, filtered and concentrated to give 61.2 (90.0 mg, crude), which was used directly to the next step.

### Synthesis of 61

To a solution of 61.2 (70.0 mg, 0.15 mmol) in acetone (5 mL) was added K₂CO₃ (42.5 mg, 0.31 mmol) and 1H-pyrazole-4-carbonitrile (17.2 mg, 0.18 mmol). The reaction mixture was stirred at RT for 2 h then quenched with saturated aq. NH₄Cl solution (30 mL). The resultant suspension was extracted with EtOAc (2×30 mL) and the combined organic phase was washed with saturated brine (50 mL), dried over anhydrous Na₂SO₄, filtered and concentrated. The residue was purified by silica gel chromatography (0-70% of EtOAc in PE) to give the crude product, which was further purified by prep-IPLC (Column: Phenomenex Gemini-NX 150*30 mm*5 um; Condition: water (0.04% NH₃H₂O+10 mM NH₄HCO₃)-ACN; Begin B: 65; End B: 95; Gradient Time (min): 8; 100% B Hold Time (min): 1) to give the product 61 (29.5 mg, 40.8%). ¹H NMR (400 MHz, CDCl₃) δ_{H} 7.86 (s, 1H), 7.81 (s, 1H), 5.09-4.91 (m, 2H), 2.59 (d, J=8.8 Hz, 1H), 2.07-2.00 (m, 1H), 1.92-1.63 (m, 9H), 1.52-1.33 (m, 7H), 1.28-1.20 (m, 10H), 1.15-1.09 (m, 6H), 1.05-0.97 (m, 1H), 0.88-0.81 (m, 4H). LC-ELSD/MS: purity>99%, MS ESI calcd. for C₂₉H₄₂N₃O [M−H₂O+H]⁺448.3, found 448.3.

### Synthesis of 62 & 63

To a solution of 60.1 (200 mg, 0.44 mmol) in acetone (10 ml) was added K₂CO₃ (121 mg, 0.88 mmol) and 5-methyl-1H-tertazole (44.4 mg, 0.53 mmol). The mixture was stirred at RT for 2 h then poured into water (20 mL) and stirred for 10 min. The resultant suspension was extracted with EtOAc (3×10 mL) and the combined organic phase was washed with saturated brine, dried over anhydrous Na₂SO₄, filtered and concentrated. The residue was purified by flash column (0˜40% of EtOAc in PE) to give 62 (65.0 mg, impure) and 63 (32.1 mg, 15.9%). The impure product 62 (65.0 mg) was further purified by prep-HPLC (Column: YMC Triart C18 150*25 mm*5 μm; Condition: water (10 mM NH₄HCO₃)-ACN; Begin B: 75; End B: 100; Gradient Time (min): 9.5; 100% B Hold Time (min): 2) to give pure product 62 (23.7 mg, 11.5%).

62: ¹H NMR (400 MHz, CDCl₃) δ 5.38 (s, 2H), 2.60-2.54 (m, 1H), 2.42-2.33 (m, 1H), 2.10-1.99 (m, 1H), 1.81-1.61 (m, 12H), 1.47-1.25 (m, 12H), 1.22-1.01 (m, 10H), 0.88-0.78 (m, 5H). LC-ELSD/MS: purity>99%, MS ESI calcd. for C₂₇H₄₃N₄₀ [M−H₂O+H]⁺ 439.3, found 439.3.

63: ¹H NMR (400 MHz, CDCl₃) δ 5.21-5.03 (m, 1H), 5.14 (d, J=2.0 Hz, 1H), 2.52-2.38 (m, 4H), 2.09-1.98 (m, 1H), 1.89-1.57 (m, 10H), 1.54-1.41 (m, 5H), 1.40-1.24 (m, 8H), 1.21-0.99 (m, 9H), 0.92-0.78 (m, 5H). LC-ELSD/MS: purity>99%, MS ESI calcd. For C₂₇H₄₃N₄₀ [M−H₂O+H]⁺439.3, found 439.3.

### Synthesis of 64 & 65

To a solution of 61.2 (140 mg, 0.35 mmol) in acetone (10 ml) was added K₂CO₃ (98.4 mg, 0.71 mmol) and methyl-1H-tertazole (39.8 mg, 0.43 mmol). The mixture was stirred at RT for 2 h then poured into water (20 mL) and stirred for 10 min. The resultant suspension was extracted with EtOAc (3×10 mL) and the combined organic phase was washed with saturated brine, dried over anhydrous Na₂SO₄, filtered and concentrated. The residue was purified by flash column (0˜40% of EtOAc in PE) to give 64 (65.0 mg, crude) and 65 (28.1 mg, 11.1%). The impure product 64 (65.0 mg) was further purified by prep-HPLC (Column: YMC Triart C18 150*25 mm*5 μm; Condition: water (10 mM NH₄HCO₃)-ACN; Begin B: 75; End B: 100; Gradient Time (min): 9.5; 100% B Hold Time (min): 2) to give pure product 64 (20.0 mg, 7.9%).

64: ¹H NMR (400 MHz, CDCl₃)^{6H} 5.50-5.32 (m, 2H), 2.63-2.59 (m, 1H), 2.56 (s, 3H), 2.08-2.00 (m, 1H), 1.96-1.63 (m, 9H), 1.54-1.43 (m, 4H), 1.41-1.21 (m, 13H), 1.18-0.96 (m, 8H), 0.87 (s, 3H). LC-ELSD/MS: purity>99%, MS ESI calcd. for C₂₇H₄₃N₄O [M−H₂O+H]⁻ 439.3, found 439.3.

65: ¹H NMR (400 MHz, CDCl₃)^{6H} 5.14 (s, 2H), 2.68-2.62 (m, 1H), 2.49 (s, 3H), 2.11-1.65 (m, 10H), 1.49-1.32 (m, 6H), 1.30-1.20 (m, 10H), 1.18-0.96 (m, 8H), 0.91-0.82 (m, 4H). LC-ELSD/MS: purity>99%, MS ESI calcd. For C₂₇H₄₃N₄₀ [M−H₂O+H]⁻ 439.3, found 439.3.

### Example 66 & 67: Synthesis of 1-((3R,5R,8R,9R,10S,13S,14S,17S)-3-hydroxy-3-methylhexadecahydro-1H-cyclopenta[alphenanthren-17-yl)ethan-1-one (66) & 1-((3R,5R,8R,9R,10S,13S,14S,17R)-3-hydroxy-3-methylhexadecahydro-1H-cyclopenta[a]phenanthren-17-yl)ethan-1-one (67)

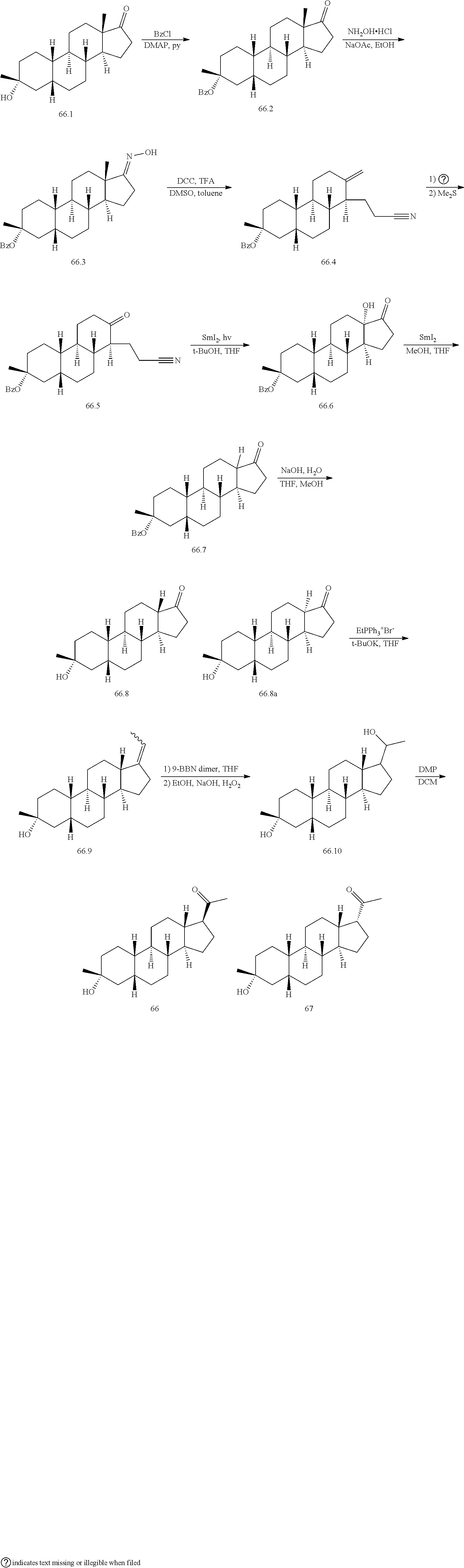

### Synthesis of 1.2

To a solution of 66.1 (25 g, 86 mmol, reported in patent WO2014/169833) and DMAP (10.5 g, 86 mmol) in pyridine (300 mL) was added drop wise benzoyl chloride (24.1 g, 172 mmol) at 20° C. After stirring at 70° C. for 12 h, the mixture was cooled and then poured into ice-water (400 mL). The reaction mixture was extracted with EtOAc (3×300 mL). The combined organic phase was washed with brine (200 mL), dried over anhydrous Na₂SO₄, filtered and concentrated. The residue was purified by flash column (0˜5% of EtOAc in PE) to give 66.2 (29 g, 86%) as an oil. ¹H NMR (400 MHz, CDCl₃)⁶H 8.00 (d, J=7.6 Hz, 2H), 7.53-7.49 (m, 1H), 7.41 (t, J=7.6 Hz, 2H), 2.49-2.40 (m, 1H), 2.19-2.04 (m, 2H), 2.01-1.88 (m, 5H), 1.86-1.77 (m, 3H), 1.62-1.52 (m, 4H), 1.51-1.09 (m, 11H), 0.88 (s, 3H) Synthesis of 66.3

To a solution of 66.2 (29 g, 73.5 mmol) in EtOH (500 mL) was added hydroxylamine hydrochloride (20.4 g, 294 mmol) and sodium acetate (24.1 g, 294 mmol) at 20° C. under N₂. After heating at 80° C. for 2 h, the mixture was cooled and concentrated. The residue was poured into water (200 mL) and stirred for 20 minutes. The aqueous phase was extracted with EtOAc (3×150 mL). The combined organic phase was washed with saturated brine (2×50 mL), dried over anhydrous Na₂SO₄, filtered and concentrated. The residue was purified by flash column (0˜20% of EtOAc in PE) to give 66.3 (21 g, 70%) as a solid. 1H NMR (400 MHz, CDCl₃) δ_{H} 8.01-7.96 (m, 2H), 7.55-7.48 (m, 1H), 7.44-7.37 (m, 2H), 2.58-2.40 (m, 2H), 2.19-2.08 (m, 2H), 2.00-1.75 (m, 8H), 1.72-1.64 (m, 4H), 1.56-1.29 (m, 8H), 1.21-1.09 (m, 3H), 0.92 (s, 3H).

### Synthesis of 66.4

To a solution of 66.3 (25 g, 61.0 mmol) and DCC (37.7 g, 183 mmol) in toluene (100 mL) and DMSO (100 mL) was added TFA (4.85 g, 42.6 mmol) dropwise at rt. After stirring at rt for 1 h, the mixture was quenched by NaHCO₃ (250 mL, sat.) and diluted with EtOAc (500 mL). The reaction mixture was filtered, and the organic layer was separated and concentrated. The residue was purified by flash column (0-2.5% EtOAc in PE) to give 66.4 (9.5 g, 40%) as an oil. ¹H NMR (400 MHz, CDCl₃) δ_{H} 8.10-7.95 (m, 2H), 7.60-7.50 (m, 1H), 7.45-7.35 (m, 2H), 4.80 (s, 1H), 4.48 (s, 1H), 2.60-1.75 (m, 12H), 1.69 (s, 3H), 1.65-1.55 (m, 3H), 1.50-1.10 (m, 5H), 1.00-0.80 (m, 3H).

### Synthesis of 66.5

To a solution of 66.4 (9.5 g, 24.2 mmol) in DCM (90 mL) and MeOH (90 mL) was added NaHCO₃ (9 g, 107 mmol). After cooling to −70° C., ozone was bubbled into the reaction mixture for approx. 30 min until a blue color persisted. The excess ozone was removed by O₂ stream until the reaction became colorless. Me₂S (9.57 g, 154 mmol) was then added and the mixture was warmed to 10° C. and stirred for 1 h. The mixture was filtered, concentrated and purified by flash column (5˜15% EtOAc in PE) to give 66.5 (5.6 g, 59%) as a solid. ¹H NMR (400 MHz, CDCl₃) δ_{H} 8.10-8.00 (m, 2H), 7.60-7.50 (m, 1H), 7.45-7.35 (m, 2H), 2.50-2.40 (m, 4H), 2.40-2.20 (m, 3H), 2.15-2.05 (m, 1H), 2.00-1.85 (m, 6H), 1.80-1.75 (m, 1H), 1.71 (s, 3H), 1.65-1.60 (m, 2H), 1.55-1.15 (m, 6H).

### Synthesis of 66.6

To a solution of 66.5 (1.4 g, 3.55 mmol) and t-BuOH (526 mg, 7.1 mmol) in THF (35 mL) was added SmI₂ (106 mL, 0.1 M in THF, 10.6 mmol) under N₂ below −20° C. The dark blue mixture was irradiated by two 275 W tungsten lamps for 1 h while being cooled by a flowing coolant to maintaining the inner temperature under −6° C. The mixture was concentrated in vacuum. The residue was diluted with HCl (25 mL, 1M) and extracted with EtOAc (2×50 mL). The combined organic layer was dried over Na₂SO₄, filtered and concentrated to give a mixture of SM (66.5) and 66.6 (1.7 g, 3:7) as an oil.

### Synthesis of 66.7

To a solution of 66.6 (6.7 g, as a mixture ˜30% 66.5) in THE (150 mL) and MeOH (30 mL) was added SmI₂ (336 mL, 0.1 M in THF, 33.6 mmol) dropwise under N₂ at 10° C. After stirring at 10° C. for more 20 min, the mixture was concentrated in vacuum. The residue was diluted HCl (100 mL, 1M) and extracted with EtOAc (2×100 mL). The combined organic layer was dried over Na₂SO₄, filtered, concentrated. The residue was purified by flash column (0-15% EtOAc in PE) to give 66.7 (3.2 g, 50%, C-13 mixture (1:1)) and recovered 66.5 (1.4 g) both as solids. ¹H NMR (400 MHz, CDCl₃) δ_{H} 8.05-7.95 (m, 2H), 7.55-7.45 (m, 1H), 7.45-7.35 (m, 2H), 2.40-1.75 (m, 12H), 1.75-1.65 (m, 3H), 1.65-0.80 (m, 11H), 0.75-0.50 (m, 1H).

### Synthesis of 66.8 & 66.18a

To a solution of 66.7 (3.2 g, 8.4 mmol) in THE (15 mL) and MeOH (30 mL) was added a solution of NaOH (1.67 g, 42 mmol) in water (15 mL). After stirring at 60° C. for 20 h, the mixture was diluted with water (20 mL) and extracted with EtOAc (2×30 mL). The combined organic layer was dried over Na₂SO₄, filtered, concentrated. The diastereomers were separated by flash column (0˜10% acetone in DCM) to give 66.8a (1.7 g) and 66.8 (0.9 g).

66.8: ¹H NMR (400 MHz, CDCl₃) δ_{H} 2.36 (dd, J=7.6, 17.2 Hz, 1H), 2.25-1.95 (m, 4H), 1.90-1.75 (m, 3H), 1.70-1.50 (m, 5H), 1.50-1.15 (m, 13H), 1.10-0.95 (m, 1H), 0.90-0.75 (m, 1H). LC-ELSD/MS: purity>99%, MS ESI calcd. for C₁₈H₂₇O [M+H-H₂O]⁺ 259.2, found 259.2

66.8a: ¹H NMR (400 MHz, CDCl₃) δ_{H} 2.30-2.10 (m, 4H), 2.05-1.95 (m, 2H), 1.90-1.65 (m, 6H), 1.60-1.40 (m, 6H), 1.35-1.05 (m, 8H), 0.65-0.45 (m, 2H). LC-ELSD/MS: purity>99%, MS ESI calcd. for C₁₈H₂₇O [M+H-H₂O]⁺259.2, found 259.1 Synthesis of 66.9

To a suspension of EtPPh₃Br (8.01 g, 21.6 mmol) in THF (40 mL) was added t-BuOK (1.81 g, 16.2 mmol). After stirring at 35° C. for 1 h. a solution of 66.8 (1.5 g, 5.42 mmol) in THF (10 mL) was added. After stirring at 35° C. for 1 h, the reaction mixture was quenched by NH₄Cl (20 mL, sat.) and extracted with EtOAc (2×50 mL). The combined organic layer was dried over Na₂SO₄, concentrated in vacuum and triturated in MTBE (40 mL) for 16 h to remove Ph₃PO. The mixture was filtered, and the filtrate was concentrated in vacuum. The residue was purified flash column (0-10% EtOAc in PE) to give 66.9 (1.7 g) as an oil. ¹H NMR (400 MHz, CDCl₃) δ_{H} 5.35-5.20 (m, 0.5H), 5.15-5.00 (m, 0.5H), 2.50-2.40 (m, 0.5H), 2.35-2.10 (m, 2H), 2.05-1.70 (m, 6.5H), 1.60-1.30 (m, 11H), 1.27 (s, 3H), 1.20-0.95 (m, 4H), 0.95-0.75 (m, 4H).

### Synthesis of 66.10

A solution of 66.9 (1.7 g, 5.89 mmol) in THF (40 mL) was added 9-BBN dimer (2.85 g, 11.7 mmol) at 10° C. After stirring at 45° C. for 3 h, ethanol (3.25 g, 70.6 mmol) was added at 15° C., followed by NaOH aqueous (14.1 mL, 5.0 M, 70.6 mmol) and H₂O₂(7.06 mL, 10 M, 70.6 mmol) dropwise at 0° C. After stirring at 60° C. for 1 h, the mixture was cooled to 15° C., diluted with Na₂SO₃ (40 mL, sat. aq) and extracted with EtOAc (2×50 mL). The combined organic solution was dried over Na₂SO₄, filtered, and concentrated. The residue was purified by flash column (15˜40% EtOAc in PE) to give 66.10 (1.8 g, 100%) as a solid. ¹H NMR (400 MHz, CDCl₃) δ_{H} 3.90-3.60 (m, 1H), 2.15-1.75 (m, 7H), 1.70-1.30 (m, 14H), 1.26 (s, 3H), 1.20-0.70 (m, 9H).

### Synthesis of 66 & 67

To a solution of 66.10 (1.8 g, 5.87 mmol) in DCM (50 mL) was added DMP (4.96 g, 11.7 mmol). After stirring at 30° C. for 2 h, the mixture was quenched with a mixture of NaHCO₃ (100 mL, sat.) and Na₂S₂O₃ (50 mL, sat.). The organic layer was separated, dried over Na₂SO₄, filtered, concentrated. The residue was purified by flash column (10˜25% EtOAc in PE) to give a mixture of diastereomers at C₁₇ (1.5 g) which were separated by flash column (0˜5% acetone in DCM) to give 66 (1.1 g) and 67 (0.18 g). The structure was determined by X-ray single crystal diffraction analysis.

66: ¹H NMR (400 MHz, CDCl₃) δ_{H} 2.50 (dt, J=7.6, 10.4 Hz, 1H), 2.12 (s, 3H), 1.95-1.75 (m, 8H), 1.70-1.60 (m, 1H), 1.55-1.20 (m, 13H), 1.15-0.95 (m, 4H), 0.90-0.70 (m, 2H). LC-ELSD/MS: purity>99%, MS ESI calcd. for C₂₀H₃₁O [M+H-H₂O]⁺287.2, found 287.3.

67: ¹H NMR (400 MHz, CDCl₃)⁶H 3.13 (dt, J=2.8, 8.8 Hz, 1H), 2.12 (s, 3H), 1.95-1.35 (m, 15H), 1.35-0.95 (m, 11H), 0.90-0.65 (m, 2H). LC-ELSD/MS: purity>99%, MS ESI calcd. for C₂₀H₃₁O [M+H-H₂O]⁺287.2, found 287.2.

### Example 68: Synthesis of 1-(2-((3R,5R,8R,9R,10S,13S,14S,17S)-3-hydroxy-3-methylhexadecahydro-1H-cyclopenta[a]phenanthren-17-yl)-2-oxoethyl)-1H-pyrazole-4-carbonitrile

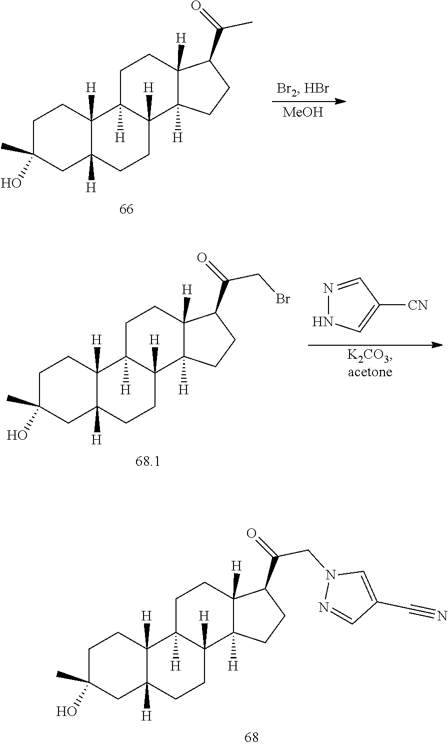

### Synthesis of 68.1

To a solution of 66 (200 mg, 0.66 mmol) in MeOH (5 mL) was added HBr (26.5 mg, 40% aq., 0.13 mmol) and Br₂ (104 mg, 0.66 mmol). After stirring at 15° C. for 2 h, the mixture was quenched with NaHCO₃ (10 mL, aq., sat.) and extracted with EtOAc (2×10 mL). The combined organic layer was dried over Na₂SO₄, filtered and concentrated to give 68.1 (250 mg, 100%) as an oil. ¹H NMR (400 MHz, CDCl₃)⁶H 3.93 (s, 2H), 2.90-2.75 (m, 1H), 2.00-1.75 (m, 8H), 1.70-1.60 (m, 1H), 1.55-1.05 (m, 17H), 0.95-0.70 (m, 2H).

### Synthesis of 68

To a solution of 68.1 (250 mg, 0.65 mmol) in acetone (5 mL) was added 4-cyano-pyrazole (91 mg, 0.98 mmol) and K₂CO₃ (179 mg, 1.3 mmol). After stirred at 15° C. for 16 h, the reaction mixture was diluted with water (20 mL) and filtered. The precipitate was washed with water (10 mL) and dried in vacuum. The residue was purified by flash column (20˜70% EtOAc in PE), re-dissolved in MeCN/water (20 mL/20 mL) and lyophilized to give 68 (135.2 mg, 52%) as a solid. ¹H NMR (400 MHz, CDCl₃) δ_{H} 7.87 (s, 1H), 7.81 (s, 1H), 5.10-4.92 (m, 2H), 2.63-2.50 (m, 1H), 2.02-1.73 (m, 8H), 1.70-1.00 (m, 18H), 0.90-0.75 (m, 2H). LC-ELSD/MS: purity>99%, MS ESI calcd. for C₂₄H₃₄N₃O₂ [M+H]⁺ 396.3, found 396.2.

### Example 69 & 70: Synthesis of 1-((3R,5R,8R,9R,10S,13S,14S,17S)-3-hydroxy-3-methylhexadecahydro-1H-cyclopenta[a]phenanthren-17-yl)-2-(5-methyl-2H-tetrazol-2-yl)ethan-1-one (69) & 1-((3R,5R,8R,9R,10S,13S,14S,17S)-3-hydroxy-3-methylhexadecahydro-1H-cyclopenta[a]phenanthren-17-yl)-2-(5-methyl-1H-tetrazol-1-yl)ethan-1-one (70)

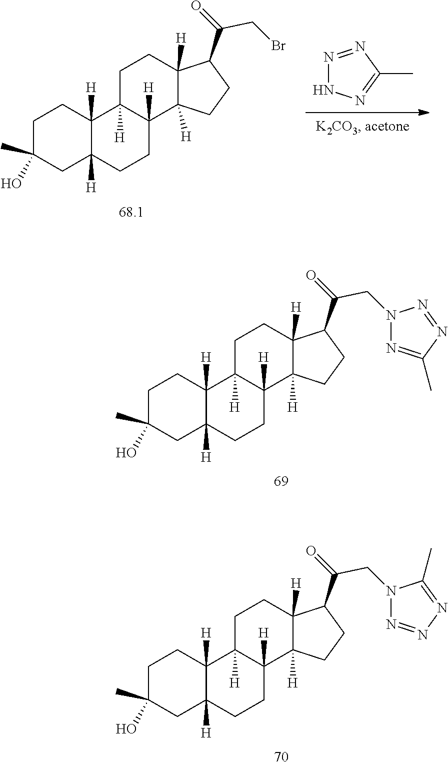

To a solution of 68.1 (220 mg, 0.57 mmol) in acetone (5 mL) were added K₂CO₃ (237 mg, 1.7 mmol) and 5-methyl-2H-1,2,3,4-tetrazole (95.8 mg, 1.1 mmol). After stirring at 20° C. for 2 h, the mixture was diluted with saturated NH₄Cl (100 mL) and extracted with EtOAc (3×30 mL). The combined organic layer was washed with saturated brine (100 mL), dried over anhydrous Na₂SO₄, filtered and concentrated. The residue was purified by flash column (0-100% of EtOAc in PE) to give 69 (59.5 mg, 27%) and 70 (98.5 mg, 45%) as solids.

69: ¹H NMR (400 MHz, CDCl₃) δ_{H} 5.45-5.35 (m, 2H), 2.60-2.45 (m, 4H), 2.00-1.60 (m, 10H), 1.52-1.28 (m, 9H), 1.27 (s, 3H), 1.25-1.00 (m, 4H), 0.90-0.75 (m, 2H). LC-ELSD/MS purity 99%, MS ESI calcd. for C₂₂H₃₅N₄O₂ [M+H]⁺ 387.3, found 387.3.

70: ¹H NMR (400 MHz, CDCl3) δ_{H} 5.21-5.10 (m, 2H), 2.70-2.55 (m, 1H), 2.46 (s, 3H), 2.05-1.60 (m, 9H), 1.52-1.28 (m, 10H), 1.27 (s, 3H), 1.25-1.05 (m, 4H), 0.90-0.75 (m, 2H). LC-ELSD/MS purity 99%, MS ESI calcd. for C₂₂H₃₅N₄O₂ [M+H]⁺ 387.3, found 387.3.

### Example 71: Synthesis of 1-(2-((3R,5R,8R,9R,10S,13S,14S,17R)-3-hydroxy-3-methylhexadecahydro-1H-cyclopenta[alphenanthren-17-yl)-2-oxoethyl)-1H-pyrazole-4-carbonitrile (6)

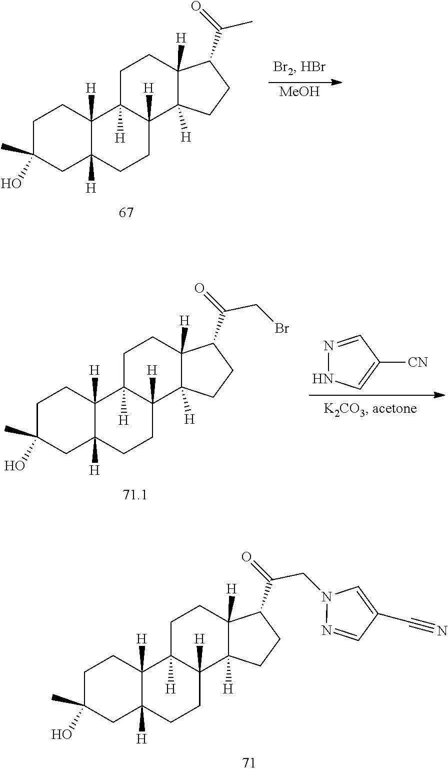

### Synthesis of 71.1

To a solution of 67 (101 mg, 0.33 mmol) in MeOH (5 mL) were added HBr (13.4 mg, 0.066 mmol, 40%) and Br₂ (58.3 mg, 0.36 mmol) at 0° C. and stirred at 15° C. for 16 hours. The resulting mixture was poured into saturated NaHCO₃/water (50 mL/50 mL). The aqueous layer was extracted with EtOAc (3×20 mL). The combined organic layer was washed with saturated brine (100 mL), dried over anhydrous Na₂SO₄, filtered and concentrated to give 71.1 (110 mg, crude). ¹H NMR (400 MHz, CDCl₃) δ_{H} 4.00-3.85 (m, 2H), 3.48-3.35 (m, 1H), 1.99-1.70 (m, 8H), 1.69-1.26 (m, 11H), 1.24 (s, 3H), 1.22-0.70 (m, 6H).

### Synthesis of 71

A solution of 71.1 (110 mg, 0.29 mmol), K₂CO₃ (79.1 mg, 0.57 mmol) and 4-cyano-pyrazole (53.4 mg, 0.57 mmol) in acetone (5 mL) was stirred at 15° C. for 2 hours. The mixture was poured into saturated brine (50 mL). The aqueous layer was extracted with EtOAc (3×20 mL). The combined organic layer was washed with water (2×100 mL), saturated brine (100 mL), dried over anhydrous Na₂SO₄, filtered and concentrated. The residue was purified by flash column (0-30% of EtOAc in PE) to give 71 (53.1 mg, 47%). ¹H NMR (400 MHz, CDCl₃) δ_{H} 7.86 (s, 1H), 7.82 (s, 1H), 5.10-4.85 (m, 2H), 3.15 (dt, J=2.8 Hz, 8.4 Hz, 1H), 2.08-1.59 (m, 10H), 1.52-1.26 (m, 9H), 1.24 (s, 3H), 1.22-0.70 (m, 6H). LC-ELSD/MS purity>99%, MS ESI calcd. for C₂₄H₃₂N₃O [M+H-H₂O]⁺378.3, found 378.2.

### Example 72: Synthesis of 1-((3R,5S,8R,9R,10S,13S,14S,17S)-3-hydroxy-3-methylhexadecahydro-1H-cyclopenta[a]phenanthren-17-yl)ethanone (72)

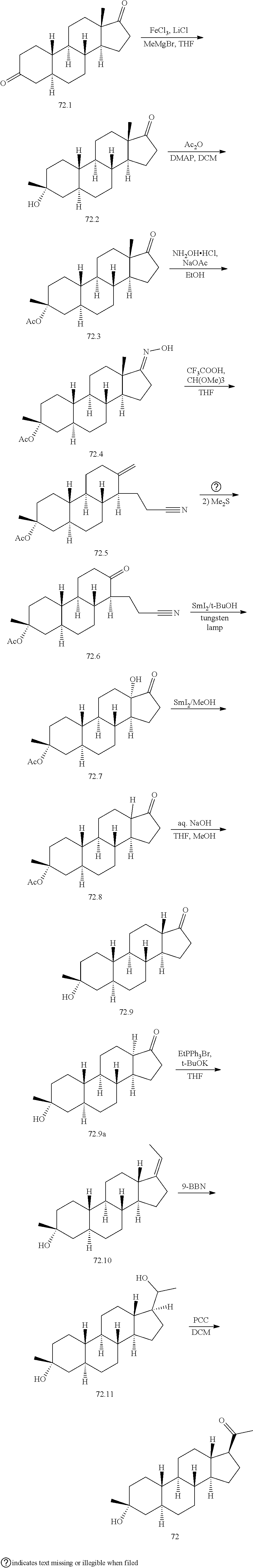

### Synthesis of 72.2

A suspension of LiCl (8.09 g, 191 mmol, anhydrous) in THE (800 mL, anhydrous) was stirred at 10° C. for 30 mins under N₂. FeCl₃ (16.2 g, 100 mmol, anhydrous) was added at 10° C. The mixture was cooled to −30° C. MeMgBr (121 mL, 3M in diethyl ether, 364 mmol) was added dropwise to the mixture at −30° C. The resulting mixture was stirred at −30° C. for 10 mins. 72.1 (25.0 g, 91.1 mmol, reported in patent ‘WO2015/180679, 2015, A1’) was added at −30° C. After stirring at −15° C. for 2 hours, the mixture was added to citric acid (500 mL, 20% aq.) and concentrated to remove most solvent. EtOAc (300 mL) was added to the residue and the resulting mixture was filtered. The solid was washed with water (100 mL) and EtOAc (100 mL), dried in vacuum to give 72.2 (20.0 g, 75.7%). ¹H NMR (400 MHz, MeOD) δ_{H} 2.50-2.37 (m, 1H), 2.14-1.20 (m, 17H), 1.16 (s, 3H), 1.12-1.02 (m, 3H), 0.89 (s, 3H), 0.79-0.67 (m, 2H).

### Synthesis of 72.3

To a solution of 72.2 (27.0 g, 92.9 mmol) and DMAP (11.3 g, 92.9 mmol) in DCM (250 mL) was added dropwise TEA (25.9 mL, 185 mmol) and Ac₂O (26.2 mL, 278 mmol) at 20° C. After stirring at 20° C. for 16 hours, the mixture was cooled and then poured into ice-water (150 mL). The aqueous phase was extracted with EtOAc (3×200 mL). The combined organic phase was washed with brine (150 mL), dried over anhydrous Na₂SO₄, filtered and concentrated. The residue was purified by flash column (0˜5% of EtOAc in PE) to give 72.3 (20.0 g, 64.9%). ¹H NMR (400 MHz, CDCl₃)⁶H 2.50-2.30 (m, 2H), 2.26-2.18 (m, 1H), 2.13-2.05 (m, 1H), 1.99 (s, 3H), 1.95-1.61 (m, 6H), 1.45 (s, 3H), 1.32-0.96 (m, 11H), 0.87 (s, 3H), 0.80-0.66 (m, 2H).

### Synthesis of 72.4

To a solution of 72.3 (20.0 g, 60.1 mmol) in EtOH (200 mL) was added hydroxylamine hydrochloride (16.6 g, 240 mmol) and sodium acetate (19.6 g, 240 mmol) at 20° C. under N₂. After refluxing at 80° C. for 16 h, the mixture was poured into water (400 mL). The mixture was filtered. The solid was washed with water (200 mL), dissolved in DCM (300 mL), dried over Na₂SO₄, filtered and concentrated in vacuum to give 72.4 (20.0 g, crude). ¹H NMR (400 MHz, CDCl₃) δ_{H} 7.86 (s, 1H), 2.55-2.40 (m, 2H), 2.38-2.30 (m, 1H), 2.25-2.15 (m, 1H), 1.99 (s, 3H), 1.89-1.63 (m, 7H), 1.45 (s, 3H), 1.42-1.29 (m, 2H), 1.21-0.97 (m, 8H), 0.91 (s, 3H), 0.77-0.68 (m, 2H).

### Synthesis of 72.5

To a stirred solution of 72.4 (20.0 g, 57.5 mmol) and CH(OMe)₃ (16.7 g, 158 mmol) in anhydrous THF (200 mL) under N₂ at 60° C. was added freshly distillated TFA (8.18 g, 71.8 mmol) in one portion. After stirring for 4 h at 65° C., the mixture was quenched by NaHCO₃ (80 mL, sat.). EtOAc (200 mL) was added to the mixture. The mixture was filtered and the organic layer was separated, concentrated and purified by flash column (0˜10% EtOAc in PE) to give 72.5 (10.0 g, 52.9%). ¹H NMR (400 MHz, CDCl₃)^{6H} 4.79 (s, 1H), 4.48 (s, 1H), 2.41-2.29 (m, 4H), 2.25-2.06 (m, 4H), 2.00 (s, 3H), 1.84-1.68 (m, 4H), 1.48-1.42 (m, 5H), 1.09-0.86 (m, 10H), 0.68-0.55 (m, 1H).

### Synthesis of 72.6

To a solution of 72.5 (14.0 g, 42.4 mmol) in DCM (140 mL) and MeOH (140 mL) was added NaHCO₃ (14.0 g, 166 mmol). The mixture was bubbled with ozone (1 atm) at −70° C. for 30 min until a blue color persisted. The excess ozone was removed by O₂ stream and the mixture turned colorless. Me₂S (1.40 g, 225 mmol) was added and the mixture was warmed to 20° C. and stirred for 16 h. The mixture was filtered, concentrated and purified by flash column (5˜40% EtOAc in PE) to give 72.6 (9.00 g, 64.2%). ¹H NMR (400 MHz, CDCl₃)⁶H 2.49-2.21 (m, 8H), 2.01 (s, 3H), 1.94-1.74 (m, 4H), 1.72-1.64 (m, 1H), 1.46 (s, 3H), 1.32-0.91 (m, 9H), 0.75-0.62 (m, 1H).

### Synthesis of 7.7

To a solution of 72.6 (1.50 g, 4.52 mmol) and t-BuOH (670 mg, 9.0 mmol) in THE (35 mL) was added SmI₂ (134 mL, 0.1 M in THF, 13.5 mmol) under N₂ below −20° C. The dark blue mixture was irradiated by two 275 W tungsten lamps for 1 h while being cooled by a flowing coolant to maintain the inner temperature under −10° C. to give a light yellow solution. The mixture was concentrated in vacuum. HCl (10 mL, 1M) was added to the residue and extracted with EtOAc (2×50 mL). The combined organic layer was dried over Na₂SO₄, filtered and concentrated to give a mixture of crude 72.6 and 72.7 (1.70 g crude). ¹H NMR (400 MHz, CDCl₃) δ_{H} 2.50-2.06 (m, 7H), 2.03-1.98 (m, 3H), 1.92-1.59 (m, 5H), 1.49-1.42 (m, 3H), 1.36-0.55 (m, 12H).

### Synthesis of 72.8

To a solution of 72.7 (8.0 g, 23.9 mmol) in THF (180 mL) and MeOH (36 mL) was added SmI₂ (477 mL, 0.1 M in THF, 47.8 mmol) dropwise under N₂ at 10° C. The blue color faded in a few seconds till getting a light yellow solution. After stirring at 10° C. for more 20 min, the mixture was concentrated in vacuum. HCl (100 mL, 2M) was added to the residue and extracted with EtOAc (2×150 mL). The combined organic layer was dried over Na₂SO₄, filtered, concentrated and purified by flash column (0-35% EtOAc in PE) to give 72.8 (1.11 g, 14.5%) and 72.6 (5.20 g). ¹H NMR (400 MHz, CDCl₃) δ_{H} 2.47-2.05 (m, 7H), 2.01-1.98 (m, 3H), 1.93-1.71 (m, 3H), 1.65-1.59 (m, 2H), 1.48-1.42 (m, 3H), 1.41-1.33 (m, 1H), 1.21-0.46 (m, 10H).

### Synthesis of 72.9 & 72.9a

To a solution of 72.8 (1.50 g, 4.71 mmol) in THF (8 mL) and MeOH (20 mL) was added a solution of NaOH (939 mg, 23.5 mmol) in water (8 mL). After stirring at 60° C. for 16 hours, the mixture was diluted with water (20 mL) and extracted with EtOAc (2×30 mL). The combined organic layer was dried over Na₂SO₄, filtered, concentrated and purified by flash column (6˜20% EtOAc in PE) to give 72.9 (200 mg, 15.3%) and 72.9a (500 mg, 38.4%, crude). To a solution of 72.9 (500 mg, 1.6 mmol, crude) in THE (5 mL) and MeOH (10 mL) was added a solution of NaOH (313 mg, 7.8 mmol) in water (5 mL). The mixture was stirred at 60° C. for 16 h. The mixture was diluted with water (20 mL) and extracted with EtOAc (2×30 mL). The combined organic layer was dried over Na₂SO₄, filtered, concentrated and purified by flash column (6-20% EtOAc in PE) to give 72.9a (290 mg, 66.9%) and 72.9 (140 mg, 32.3%).

72.9: ¹H NMR (400 MHz, CDCl₃) δ_{H} 2.44-2.29 (m, 1H), 2.20-1.99 (m, 4H), 1.95-1.86 (m, 1H), 1.82-1.74 (m, 1H), 1.72-1.53 (m, 5H), 1.45-1.29 (m, 3H), 1.21 (s, 3H), 1.19-0.94 (m, 7H), 0.91-0.73 (m, 2H), 0.70-0.58 (m, 1H). LC-ELSD/MS: purity>99%, MS ESI calcd. for C₁₈H₂₇O [M+H-H₂O]⁺ 259.2, found 259.2.

72.9a: ¹H NMR (400 MHz, CDCl₃) δ_{H} 2.29-2.16 (m, 3H), 2.02-1.93 (m, 3H), 1.86-1.73 (m, 3H), 1.67-1.56 (m, 2H), 1.55 (s, 3H), 1.48-1.26 (m, 3H), 1.20 (s, 3H), 1.16-0.97 (m, 4H), 0.75-0.43 (m, 4H). LC-ELSD/MS: purity>99%, MS ESI calcd. for C₁₈H₂₇O [M+H-H₂O]⁺259.2, found 259.2.

### Synthesis of 72.10

To a mixture of EtPPh₃Br (1.19 g, 3.2 mmol) in THF (5 mL) was added t-BuOK (362 mg, 3.2 mmol) at 25° C. under N₂. The resulting mixture was stirred at 40° C. for 30 min.

72.9 (300 mg, 1.1 mmol) was added at 40° C. After stirring at 40° C. for 1 hour to give a yellow suspension, the reaction mixture was quenched with saturated NH₄Cl aqueous (30 mL) at 20° C. The aqueous layer was extracted with EtOAc (2×50 mL). The combined organic phase was concentrated. The residue was purified by flash column (0˜20% of EtOAc in PE) to give 72.10 (250 mg, 80.3%). ¹H NMR (400 MHz, CDCl₃) OH 5.34-5.03 (m, 1H), 2.50-1.56 (m, 13H), 1.54-1.49 (m, 1H), 1.39-1.22 (m, 3H), 1.20 (s, 3H), 1.19-0.55 (m, 11H).

### Synthesis of 72.11

To a solution of 72.10 (250 mg, 0.9 mmol) in anhydrous THE (3 mL) was added 9-BBN dimer (422 mg, 1.7 mmol) at 25° C. under N₂. The mixture was stirred at 45° C. for 16 h. To the resulting mixture was added ethanol (1.00 mL, 17.3 mmol) at 15° C., followed by NaOH aqueous (691 mg in 3.46 mL of water, 5.0 M, 17.3 mmol) and H₂O₂(1.73 mL, 10 M, 17.3 mmol) was added dropwise at 0° C. After stirring at 60° C. for 1 hour. The mixture was cooled to 15° C. and Na₂SO₃ (20 mL, sat. aq.) was added. The mixture was extracted with EtOAc (2×50 mL). The combined organic layer was dried over Na₂SO₄, filtered, concentrated and purified by flash column (15˜40% EtOAc in PE) to give 72.11 (200 mg, 75.4%). ¹H NMR (400 MHz, CDCl₃) δ_{H} 3.95-3.61 (m, 1H), 2.15-1.92 (m, 2H), 1.85-1.56 (m, 7H), 1.54-1.51 (m, 1H), 1.40-1.10 (m, 12H), 1.09-0.53 (m, 10H).

### Synthesis of 72

To a solution of 72.11 (200 mg, 0.7 mmol) in DCM (20 mL) at 0° C. was added silica gel (300 mg) and PCC (280 mg, 1.3 mmol). The mixture was stirred at 25° C. for 2 h. PE (20 mL) was added to the reaction mixture. The resulting mixture was filtered through a pad of silica gel and the filter cake was washed with EtOAc (4×40 mL). The filtrate was concentrated and the residue was purified by flash column (5%˜20% of EtOAc in PE) two times to give 7 (107 mg, 55.5%). ¹H NMR (400 MHz, CDCl₃) δ_{H} 2.57-2.45 (m, 1H), 2.18-2.10 (m, 3H), 2.01-1.73 (m, 7H), 1.70-1.62 (m, 1H), 1.54-1.50 (m, 1H), 1.42-1.28 (m, 3H), 1.20 (s, 3H), 1.18-0.95 (m, 8H), 0.92-0.56 (m, 5H). LC-ELSD/MS: purity>99%, MS ESI calcd. for C₂₀H₃₁O [M+H-H₂O]⁺287.2, found 287.2.

### Example 73 & 76: Synthesis of 1-((3R,5S,8R,9R,10S,13S,14S,17S)-3-hydroxy-3-methylhexadecahydro-1H-cyclopenta[a]phenanthren-17-yl)-2-(5-methyl-2H-tetrazol-2-yl)ethanone (8) & 1-((3R,5S,8R,9R,10S,13S,14S,17S)-3-hydroxy-3-methylhexadecahydro-1H-cyclopenta[a]phenanthren-17-yl)-2-(5-methyl-1H-tetrazol-1-yl)ethanone (76)

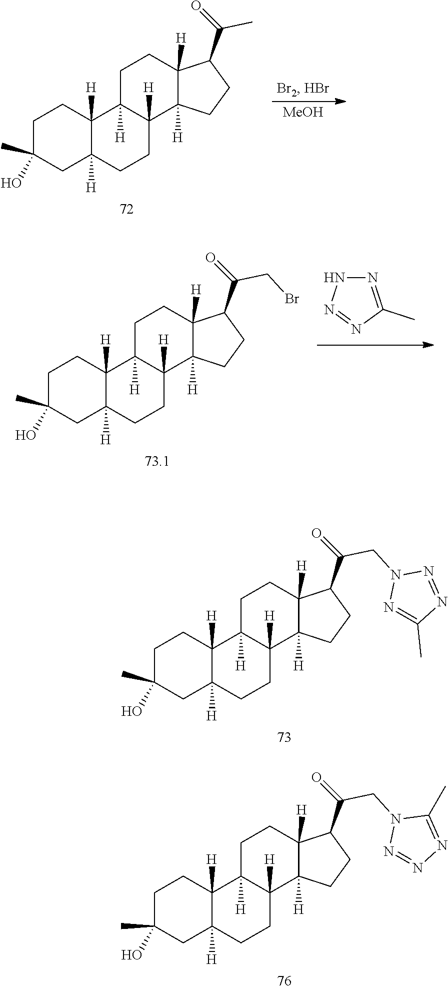

### Synthesis of 73.1

To a solution of 72 (100 mg, 0.3 mmol) in MeOH (3 mL) was added HBr (6.62 mg, 0.03 mmol, 40% in water) and Br₂ (52.4 mg, 0.3 mmol) at 25° C. After stirring at 25° C. for 2 hrs. The mixture was poured into NaHCO₃ (10 mL, saturated), extracted with EtOAc (2×10 mL). The organic layer was separated, dried over Na₂SO₄, filtered and concentrated in vacuum to give 73.1 (160 mg, crude).

### Synthesis of 73 & 76

To a solution of 73.1 (100 mg, 0.26 mmol) in acetone (3 mL) was added 5-methyl-1H-1,2,3,4-tetrazole (43.8 mg, 0.5 mmol), K₂CO₃ (108 mg, 0.78 mmol). After stirring at 25° C. for 16 hours, the mixture was added water (10 mL) and extracted with EtOAc (2×20 mL). The organic layer was separated, dried over Na₂SO₄, filtered and concentrated. The residue was purified by flash column (20˜80% of EtOAc in PE) to give 73 (9.6 mg, 9.59%) and 76 (50 mg, 50.0%). Compound 76 (50 mg, 0.1293 mmol) was further purified by SFC (column: DAICEL CHIRALPAK AS (250 mm*30 mm, 10 μm)); Mobile phase: A: CO2 B: 0.1% NH₃H₂O ETOH; gradient: from 25% to 25% of B, FlowRate (ml/min): 60) to give 76 (1.3 mg, 2.6%).

73: ¹H NMR (400 MHz, CDCl₃)⁶H 5.45-5.34 (m, 2H), 2.57 (s, 3H), 2.07-1.57 (m, 10H), 1.54-1.52 (m, 1H), 1.41-1.24 (m, 3H), 1.20 (s, 3H), 1.17-0.55 (m, 12H). LC-ELSD/MS purity>99%, MS ESI calcd. for C₂₂H₃₅N₄O₂ [M+H]⁻ 387.3, found 387.3.

76: ¹H NMR (400 MHz, CDCl₃) δ_{H} 5.32-4.92 (m, 2H), 3.26-3.18 (m, 1H), 2.48 (s, 3H), 2.15-1.64 (m, 7H), 1.35-1.23 (m, 5H), 1.19 (s, 4H), 1.15-0.57 (m, 12H). LC-ELSD/MS purity>99%, MS ESI calcd. for C₂₂H₃₃N₄₀ [M+H-H₂O]⁺ 369.3, found 369.3.

### Example 74: Synthesis of 1-((3R,5S,8R,9R,10S,13S,14S,17S)-3-hydroxy-3-methylhexadecahydro-1H-cyclopenta[a]phenanthren-17-yl)-2-(5-methyl-2H-tetrazol-2-yl)ethanone (74)

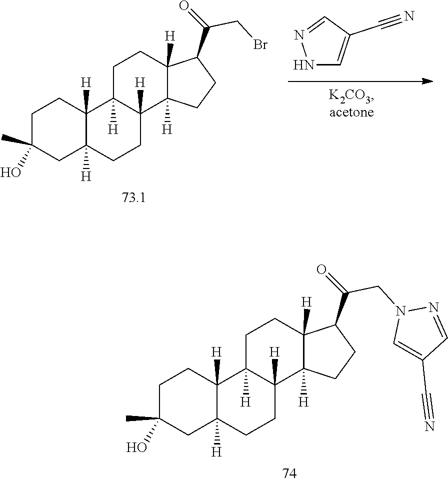

### Synthesis of 74

To a solution of 73.1 (60.0 mg, 0.2 mmol) in acetone (1 mL) was added 4-cyanopyrazole (29.1 mg, 0.3 mmol) and K₂CO₃ (43.2 mg, 0.313 mmol). After stirring at 25° C. for 16 h, water (10 mL) was added and the resulting mixture was extracted with EtOAc (2×10 mL). The organic layer was separated, concentrated and purified by flash column (10-40% EtOAc in PE) to give 74 (10.8 mg, 17.4%). ¹H NMR (400 MHz, CDCl₃) δ_{H} 7.87 (s, 1H), 7.82 (s, 1H), 5.09-4.94 (m, 2H), 2.62-2.50 (m, 1H), 2.04-1.84 (m, 5H), 1.82-1.73 (m, 2H), 1.70-1.62 (m, 1H), 1.39-1.23 (m, 4H), 1.20 (s, 3H), 1.17-0.95 (m, 8H), 0.93-0.60 (m, 5H). LC-ELSD/MS purity>99%, MS ESI calcd. for C₂₄H₃₄N₃O₂ [M+H]⁺ 396.3, found 396.3.

### Example 75: Synthesis of 1-((1R,4aS,4bR,6aR,8R,10aS,10bR,12aS)-8-hydroxy-8-methyloctadecahydrochrysen-1-yl)-ethanone (75)

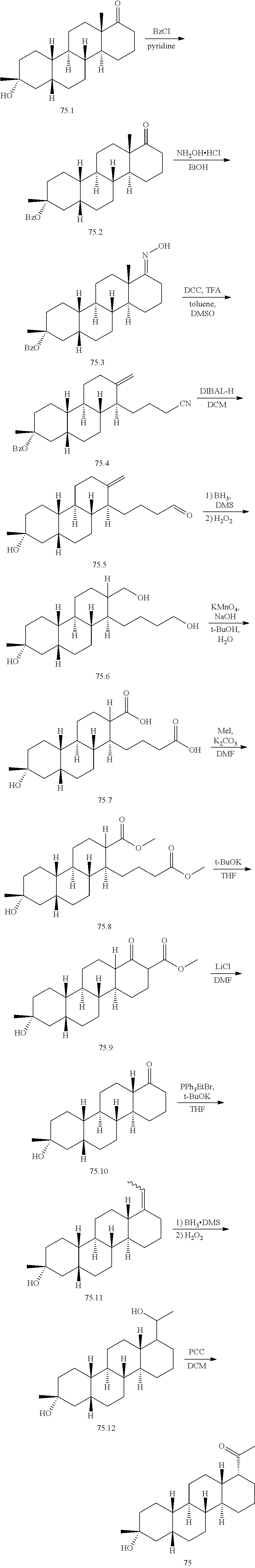

### Synthesis of 75.2

To a solution of 75.1 (10 g, 32.8 mmol, reported in patent ‘WO2014/169833, 2014, A1’) in pyridine (150 mL) was added DMAP (4.0 g, 32.8 mmol) and BzCl (9.22 g, 65.6 mmol). After stirring at 70° C. for 16h, water (200 mL) was added and the mixture was concentrated in vacuum. To the residue was added NaHCO₃ (100 mL, sat.) and extracted with EtOAc (2×100 mL). The combined organic layer was dried over Na₂SO₄, filtered, concentrated and purified by flash column (0-6% EtOAc in PE) to give 75.2 (4.10 g, 30%). ¹H NMR (400 MHz, CDCl₃) δ_{H} 8.05-7.95 (m, 2H), 7.55-7.45 (m, 1H), 7.45-7.35 (m, 2H), 2.61 (dt, J=6.8, 14.0 Hz, 1H), 2.25-2.15 (m, 1H), 2.15-1.75 (m, 8H), 1.69 (s, 3H), 1.60-1.50 (m, 3H), 1.50-1.15 (m, 9H), 1.09 (s, 3H), 1.05-0.80 (m, 3H).

### Synthesis of 75.3

To a solution of 75.2 (4.10 g, 10.0 mmol) in EtOH (50 mL) was added hydroxylamine hydrochloride (1.73 g, 25.0 mmol) and sodium acetate (2.05 g, 25.0 mmol). After stirring at 80° C. for 4 hours, the mixture was poured into water (400 mL). The mixture was then filtered. The solid was washed with water (50 mL), dissolved in DCM (50 mL), dried over Na₂SO₄, filtered and concentrated in vacuum to give 75.3 (4.40 g, crude). 1H NMR (400 MHz, CDCl₃) δ_{H} 9.09 (br., 1H), 8.05-7.95 (m, 2H), 7.55-7.45 (m, 1H), 7.45-7.35 (m, 2H), 3.30-3.20 (m, 1H), 2.15-2.00 (m, 1H), 2.00-1.75 (m, 10H), 1.68 (s, 3H), 1.65-1.50 (m, 5H), 1.50-0.90 (m, 11H).

### Synthesis of 75.4

To a solution of 75.3 (2.40 g, 5.66 mmol) and DCC (3.48 g, 16.9 mmol) in toluene (10 mL) and DMSO (10 mL) was added TFA (388 mg, 3.96 mmol) dropwise at 10° C. After stirring at 15° C. for 1 h, the mixture was quenched by NaHCO₃ (50 mL, sat.). EtOAc (100 mL) was added to the mixture. The resulting mixture was filtered and the organic layer was separated, concentrated and purified by flash column (0-5% EtOAc in PE) to give 75.4 (1.08 g, 47%). ¹H NMR (400 MHz, CDCl₃)^{6H} 8.05-7.95 (m, 2H), 7.55-7.45 (m, 1H), 7.45-7.35 (m, 2H), 4.76 (s, 1H), 4.53 (s, 1H), 2.40-2.25 (m, 2H), 2.20-2.10 (m, 1H), 2.05-1.75 (m, 9H), 1.69 (s, 3H), 1.65-1.50 (m, 6H), 1.50-1.00 (m, 7H).

### Synthesis of 75.5

To a solution of 75.4 (1.53 g, 3.77 mmol) in DCM (25 mL) was added DIBAL-H (13.1 mL, 1 M in toluene, 13.1 mmol) at −70° C. After stirring at −70° C. for 1 h, the mixture was poured into HCl (20 mL, 2 M). The organic layer was separated, dried over Na₂SO₄, filtered, concentrated and purified by flash column (10˜20% EtOAc in PE) to give 75.5 (630 mg, 55%). ¹H NMR (400 MHz, CDCl₃) OH 9.80-9.75 (m, 1H), 4.75 (s, 1H), 4.57 (s, 1H), 2.50-2.30 (m, 3H), 2.00-1.70 (m, 7H), 1.70-1.40 (m, 10H), 1.35-1.20 (m, 6H), 1.20-1.10 (m, 1H), 0.95-0.80 (m, 2H).

### Synthesis of 75.6

To a solution of 75.5 (420 mg, 1.37 mmol) in THE (10 mL) was added BH₃·Me₂S (1.37 mL, 10 M, 13.7 mmol) under N₂ at 0° C. The mixture was stirred at 20° C. for 1 h. To the mixture was added EtOH (1.89 g, 41.1 mmol), NaOH (8.22 mL, SM, 41.1 mmol) and H₂O₂ (2.74 mL, 10 M, 27.4 mmol). After stirring at 20° C. for 1 h, the mixture was quenched with Na₂SO₃ (10%, 40 mL) and extracted with EtOAc (2×40 mL). The combined organic layer was dried over Na₂SO₄, filtered and concentrated in vacuum to give 75.6 (500 mg, crude).

### Synthesis of 75.7

To a solution of 75.6 (500 mg, 1.54 mmol) in t-BuOH (30 mL) was added NaOH (20 mL, 4 M, 80 mmol) and KMnO₄ (2.9 g, 18.4 mmol). After stirring at 20° C. for 16 h, the mixture was quenched by Na₂SO₃ (10%, 60 mL) followed by HCl (120 mL, 2 M) below 20° C. The mixture was extracted with EtOAc (2×150 mL). The combined organic layer was dried over Na₂SO₄, filtered and concentrated in vacuum to give 75.7 (300 mg).

### Synthesis of 75.8

To a solution of 75.7 (200 mg, 0.56 mmol) in DMF (5 mL) was added K₂CO₃ (795 mg, 5.67 mmol) and MeI (1.6 g, 11.3 mmol). After stirring at 20° C. for 16 h, EtOAc (20 mL) was added and the resulting mixture washed with water (20 mL). The organic layer was dried over Na₂SO₄, filtered, concentrated and purified by flash column (0-30% EtOAc in PE) to give 75.8 (200 mg).

### Synthesis of 75.9

To a solution of 75.8 (100 mg, 0.26 mmol) in THF (10 mL) was added t-BuOK (293 mg, 2.62 mmol). After heating under reflux at 70° C. under N₂ for 1 h, the mixture was quenched by NH₄Cl (20 mL, sat) and extracted with EtOAc (2×20 mL). The combined organic layer was dried over Na₂SO₄, filtered and concentrated in vacuum to give 75.9 (100 mg, crude).

### Synthesis of 75.10

To a solution of 75.9 (100 mg, 0.28 mmol) in DMF (1 mL) and H₂O (0.02 mL) was added LiCl (60.6 mg, 1.43 mmol), the reaction mixture was then heated at 150° C. for 3 h. The mixture was combined with another batch from 100 mg 75.9, diluted with EtOAc (10 mL), washed with LiCl (10 mL, 3%), dried over Na₂SO₄, filtered, concentrated and purified by flash column (10-20% EtOAc in PE for first time and 0˜5% acetone in DCM for the second time) to give 75.10 (60 mg). ¹H NMR (400 MHz, CDCl₃) δ_{H} 2.40-2.25 (m, 2H), 2.20-2.05 (m, 2H), 2.00-1.90 (m, 4H), 1.80-1.70 (m, 3H), 1.70-1.60 (m, 2H), 1.50-1.05 (m, 15H), 1.00-0.90 (m, 1H), 0.80-0.70 (m, 1H). LC-ELSD/MS purity>99%, MS ESI calcd. for C₁₉H₂₉O [M+H-H₂O]273.2, found 273.2.

### Synthesis of 75.11

To a suspension of Ph₃PEtBr (1.4 g, 3.78 mmol) in THE (5 mL) was added t-BuOK (424 mg, 3.78 mmol). The mixture was stirred at 40° C. for 1 h. To the mixture was added a solution of 75.10 (110 mg, 0.38 mmol) in THF (1 mL). After stirring at 60° C. for 16 h, the mixture was quenched with NH₄Cl (10 mL) and extracted with EtOAc (20 mL). The organic layer was separated, concentrated and purified by flash column (0-8% EtOAc in PE) to give 75.11 (70 mg, 61%). ¹H NMR (400 MHz, CDCl₃) δ_{H} 5.07 (q, J=6.4 Hz, 1H), 2.75-2.65 (m, 1H), 2.10-1.70 (m, 7H), 1.70-1.60 (m, 4H), 1.50-1.10 (m, 13H), 1.10-0.70 (m, 8H).

### Synthesis of 75.12

To a solution of 75.11 (70 mg, 0.23 mmol) in THE (5 mL) was added BH₃·Me₂S (0.462 mL, 10 M). The mixture was stirred at 20° C. for 16 h. To the mixture was added EtOH (1.32 mL), NaOH (4.62 mL, 5 M) and H₂O₂(0.462 mL, 10 M). After stirring at 20° C. for 1 h, the mixture was quenched by Na₂SO₃ (10 mL, 10%) and extracted with EtOAc (20 mL). The organic layer was dried over Na₂SO₄, filtered and concentrated in vacuum to give 75.12 (100 mg, crude).

### Synthesis of 75

To a solution of 75.12 (100 mg, 0.31 mmol) in DCM (2 mL) was added silica gel (300 mg) and PCC (134 mg). The mixture was stirred at 20° C. for 2 h. The mixture was concentrated and purified by flash column (10˜20% EtOAc in PE) to give 75 (60.0 mg). ¹H NMR (400 MHz, CDCl₃) δ_{H} 2.80-2.75 (m, 1H), 2.11 (s, 3H), 2.05-1.95 (m, 1H), 1.95-1.80 (m, 3H), 1.80-1.60 (m, 4H), 1.55-1.20 (m, 17H), 1.15-1.00 (m, 2H), 0.80-0.50 (m, 3H). LC-ELSD/MS: purity>99%, MS ESI calcd. for C₂₁H₃₃O [M+H-H₂O]⁺ 301.3, found 301.3.

### Example 77: Synthesis of 1-((1S,4aS,4bR,6aR,8R,10aS,10bR,12aS)-8-hydroxy-8-methyloctadecahydrochrysen-1-yl)-ethanone (77)

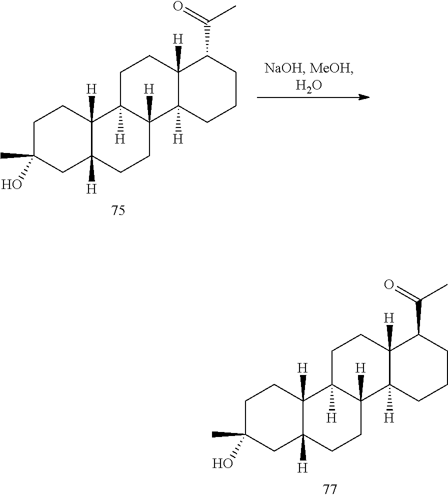

### Synthesis of 77

To a solution of 75 (30.0 mg, 0.09 mmol) in MeOH (2 mL) was added NaOH (50 mg in 0.5 mL water). After stirring at 60° C. for 16 hours, water (10 mL) was added to the mixture and extracted with EtOAc (2×10 mL). The combined organic layer was dried over Na₂SO₄, filtered and concentrated to give 77. The crude was further purified by flash column (10-25% EtOAc in PE) and lyophilized to give 77 (30 mg). ¹H NMR (400 MHz, CDCl₃)⁶H 2.25-2.15 (m, 1H), 2.12 (s, 3H), 2.00-1.90 (m, 1H), 1.90-1.70 (m, 7H), 1.55-1.20 (m, 15H), 1.10-0.70 (m, 7H). LC-ELSD/MS: purity>99%, MS ESI calcd. for C₂₁H₃₃O [M+H-H₂O]⁺ 301.3, found 301.3.

### Example 78: Synthesis of 1-(2-((1S,4aS,4bR,6aR,8R,10aS,10bR,12aS)-8-hydroxy-8-methyloctadecahydrochrysen-1-yl)-2-oxoethyl)-1H-pyrazole-4-carbonitrile (78)

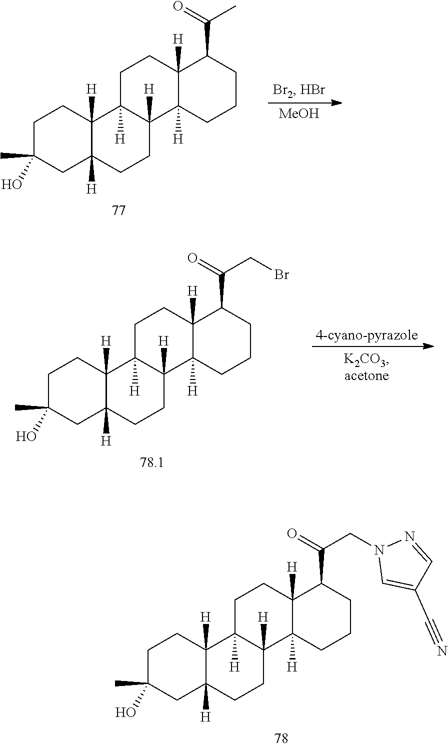

### Synthesis of 78.1

To a solution of 77 (50.0 mg, 0.15 mmol) in MeOH (3 mL) was added HBr (6.3 mg, 40%, 0.03 mmol) and Br₂ (25 mg, 0.15 mmol). After stirring at 20° C. for 1 h, NaHCO₃ (10 mL, sat.) was added to the mixture followed by EtOAc (10 mL). The organic layer was separated, dried over Na₂SO₄, filtered and concentrated to give 78.1 (70 mg, crude). ¹H NMR (400 MHz, CDCl₃) δ_{H} 3.93 (s, 2H), 2.55-2.45 (m, 1H), 2.00-1.65 (m, 9H), 1.50-1.20 (m, 15H), 1.10-0.70 (m, 6H).

### Synthesis of 78

To a solution of 78.1 (70 mg, 0.17 mmol) in acetone (1 mL) was added K₂CO₃ (48.6 mg, 0.35 mmol) and 4-cyano-pyrazole (19.6 mg, 0.21 mmol). After stirring at 20° C. for 16 h, water (10 mL) and EtOAc (10 mL) were added to the mixture. The organic layer was separated, dried over Na₂SO₄, filtered, concentrated, purified by flash column (10˜40% EtOAc in PE), dissolved in MeCN/water (10 mL/10 mL) and lyophilized to give 78 (43.4 mg, 60%). ¹H NMR (400 MHz, CDCl₃) δ_{H} 7.86 (s, 1H), 7.82 (s, 1H), 5.06 (d, J=18.4 Hz, 1H), 4.93 (d, J=18.0 Hz, 1H), 2.26 (dt, J=3.2 Hz, 11.6 Hz, 1H), 2.05-1.65 (m, 9H), 1.55-1.20 (m, 1511), 1.10-0.95 (m, 211), 0.95-0.70 (m, 4H). LC-ELSD/MS purity>99%, MS ESI calcd. for C₂₅H₃₆N₃O₂ [M+H]⁺ 410.3, found 410.3.

### Example 79: Synthesis of 1-((1S,4aS,4bR,6aS,8R,10aS,10bR,12aS)-8-hydroxy-8-methyloctadecahydrochrysen-1-yl)-ethanone (79)

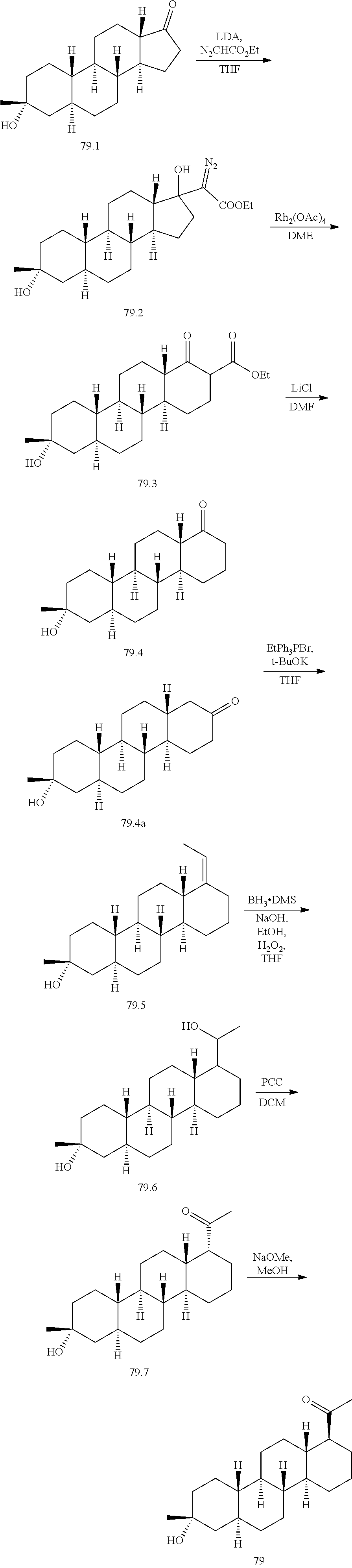

### Synthesis of 79.2

To a solution of DIPA (1.27 mL, 9.0 mmol) in THE (10 mL) was added n-BuLi (3.60 mL, 2.5 M in hexane, 9.0 mmol) at −70° C. The mixture was warmed to 25° C. and stirred for 1 h. The fresh prepared LDA (9.0 mmol) solution in THF (10 mL) was added to a stirred solution of 79.1 (500 mg, 1.8 mmol) and ethyl diazoacetate (1.13 g, 9.0 mmol, 90%) in THF (10 mL) at −70° C. The mixture was stirred at −70° C. for 2 hours. Then acetic acid (540 mg, 93.0 mmol) in THF (5 mL) was added and the mixture was then warm to 20° C. for 16 hrs. Water (300 mL) was added. The aqueous phase was extracted with EtOAc (3×100 mL). The combined organic layers were washed with brine (300 mL), dried over anhydrous Na₂SO₄, filtered and concentrated and was purified by flash column (0-20% of EtOAc in PE) to give 79.2 (500 mg, 71.2%). ¹H NMR (400 MHz, CDCl₃) δ_{H} 4.24 (q, 0.1=7.2 Hz, 2H), 3.73-3.62 (m, 1H), 2.17-2.08 (m, 1H), 1.95-1.60 (m, 10H), 1.54-1.49 (m, 2H), 1.38-1.23 (m, 6H), 1.20 (s, 4H), 1.17-0.53 (m, 8H).

### Synthesis of 79.3

To a solution of 79.2 (500 mg, 1.28 mmol) in DME (5 mL) was added Rh₂(OAc)₄ (56.5 mg, 0.1280 mmol) in one portion at 25° C. After stirring at 25° C. for 16 hours, the mixture was poured into water (20 mL) and extracted with EtOAc (2×30 mL). The combined organic layers were washed with brine (30 mL), dried over Na₂SO₄, filtered and concentrated to give 79.3 (440 mg, crude).

### Synthesis of 79.4

To a solution of 79.3 (440 mg, 1.21 mmol) in DMF (5 mL) and H₂O (0.1 mL) was added LiCl (256 mg, 6.05 mmol). After stirring at 150° C. for 3 hours, the mixture was diluted with EtOAc (10 mL), washed with LiCl (10 mL, 3%), dried over Na₂SO₄, filtered, concentrated and purified by flash column (0-5% Acetone in DCM) to give 79.4a (30 mg, 8.54%) and 79.4 (10 mg, 2.84%) and 79.4 & 79.4a (250 mg, mixture). To a solution of 79.4 & 79.4a (250 mg, 0.86 mmol) in THF (2 mL) and MeOH (10 mL) was added a solution of NaOH (343 mg) in water (1.0 mL). After stirring at 60° C. for 16 hours, the mixture was then diluted with water (20 mL) and extracted with EtOAc (2×30 mL). The combined organic layer was dried over Na₂SO₄, filtered, concentrated and purified by flash column (0˜4% Acetone in DCM) to give 79.4a (60 mg, 24.0%) and 79.4 (190 mg, 76.3%).

79.4a: ¹H NMR (400 MHz, CDCl₃)^{6H} 2.64-2.51 (m, 1H), 2.35-2.02 (m, 5H), 1.96-1.59 (m, 8H), 1.54-1.24 (m, 5H), 1.23-1.18 (m, 3H), 1.17-0.79 (m, 7H), 0.75-0.56 (m, 1H).

79.4: ¹H NMR (400 MHz, CDCl₃) δ_{H} 2.41-2.23 (m, 2H), 2.17-1.90 (m, 6H), 1.85-1.74 (m, 1H), 1.72-1.58 (m, 3H), 1.56-1.52 (m, 1H), 1.37-1.21 (m, 4H), 1.20 (s, 3H), 1.19-0.71 (m, 9H), 0.63-0.51 (m, 1H). LC-ELSD/MS: purity>99%, MS ESI calcd. for C₁₉H₃₁O₂ [M+H]⁺ 291.2, found 291.2.

### Synthesis of 79.5

To a mixture of EtPPh₃Br (1.21 g, 3.27 mmol) in THE (5 mL) was added t-BuOK (366 mg, 3.27 mmol) at 25° C. under N₂. The resulting mixture was stirred at 40° C. for 30 min. Then 79.4 (190 mg, 0.6541 mmol) was added and continue stirring at 40° C. for 16 hours. The reaction mixture was quenched with saturated NH₄C1 aqueous (50 mL) at 20° C. The aqueous was extracted with EtOAc (2×50 mL). The combined organic phase was concentrated. The residue was purified by flash column (0˜15% of EtOAc in PE) to give 79.5 (130 mg, 65.9%). ¹H NMR (400 MHz, CDCl₃) δ_{H} 5.13-5.04 (m, 1H), 2.77-2.58 (m, 1H), 2.11-1.77 (m, 7H), 1.72-1.63 (m, 1H), 1.62-1.57 (m, 4H), 1.39-1.24 (m, 3H), 1.20 (s, 5H), 1.17-0.67 (m, 11H), 0.64-0.50 (m, 1H).

### Synthesis of 79.6

To a solution of 79.5 (130 mg, 0.4297 mmol) in THE (2 mL) was added BH₃·Me₂S (214 μL, 10 M, 2.14 mmol) and the mixture was stirred at 25° C. for 16 h. To the mixture was added EtOH (500 μL, 8.59 mmol) dropwise followed by NaOH (1.71 mL, 5 M, 8.59 mmol) and H₂O₂(859 μL, 10 M, 8.59 mmol). After stirring at 70° C. for 2 hours, the mixture was quenched with Na₂SO₃ (100 mL, 10%) and extracted with EtOAc (2×100 mL). The organic layer was separated, dried with Na₂SO₄, filtered and concentrated in vacuum to give 79.6 (200 mg, crude).

### Synthesis of 79.7

To a solution of 79.6 (200 mg, 0.6 mmol) in DCM (6 mL) at 0° C. was added silica gel (300 mg) and PCC (267 mg, 1.24 mmol). After stirring at 25° C. for 1 h. The mixture was concentrated and purified by flash column (0%-15% of EtOAc in PE) to give 79.7 (130 mg, 65.6%). ¹H NMR (400 MHz, CDCl₃) δ_{H} 2.86-2.72 (m, 1H), 2.10 (s, 3H), 2.01-1.59 (m, 7H), 1.51-1.24 (m, 8H), 1.20-1.18 (m, 3H), 1.14-0.46 (m, 12H).

### Synthesis of 79

To a solution of 79.7 (130 mg, 0.4081 mmol) in THF (2 mL) and MeOH (4 mL) was added a solution of NaOH (2.04 mL, 6.12 mmol, 3 M). After stirring at 60° C. for 16 hours, the mixture was diluted with water (20 mL) and extracted with EtOAc (2×30 mL). The combined organic layer was dried over Na₂SO₄, filtered, concentrated and purified by flash column (6-25% EtOAc in PE) to give 79 (100 mg, 77.5%). ¹H NMR (400 MHz, CDCl₃) δ_{H} 2.21-2.14 (m, 1H), 2.12 (s, 3H), 2.00-1.90 (m, 3H), 1.87-1.73 (m, 3H), 1.70-1.57 (m, 3H), 1.54-1.51 (m, 1H), 1.37-1.24 (m, 5H), 1.20 (s, 3H), 1.18-1.16 (m, 1H), 1.09-0.69 (m, 10H), 0.63-0.49 (m, 1H). LC-ELSD/MIS: purity>97%, MS ESI calcd. for C₂₁H₃₃O [M+H-H₂O]⁺ 301.3, found 301.3.

### Example 80 & 81: Synthesis of 1-((1S,4aS,4bR,6aS,8R,10aS,10bR,12aS)-8-hydroxy-8-methyloctadecahydrochrysen-1-yl)-2-(5-methyl-2H-tetrazol-2-yl)ethanone (80) 1-((1S,4aS,4bR,6aS,8R,10aS,10bR,12aS)-8-hydroxy-8-methyloctadecahydrochrysen-1-yl)-2-(5-methyl-1H-tetrazol-1-yl)ethanone (81)

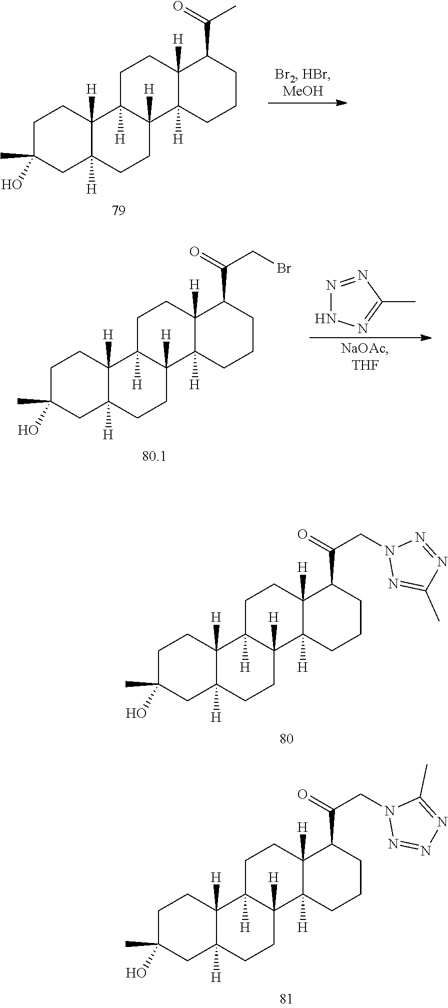

### Synthesis of 80.1

To a solution of 80 (90 mg, 0.28 mmol) in MeOH (2 mL) was added HBr (11.4 mg, 0.056 mmol, 40% in water) and Br₂ (45.1 mg, 0.28 mmol) at 25° C. After stirring at 25° C. for 3 hrs, the mixture was poured into NaHCO₃ (10 mL, saturated) and extracted with EtOAc (2×10 mL). The organic layer was separated, dried over Na₂SO₄, filtered and concentrated in vacuum to give 80.1 (130 mg, crude). ¹H NMR (400 MHz, CDCl₃) δ_{H} 3.98-3.88 (m, 2H), 2.58-2.46 (m, 1H), 2.01-1.59 (m, 8H), 1.37-1.23 (m, 6H), 1.20 (s, 3H), 1.17-0.73 (m, 12H), 0.61-0.48 (m, 1H).

### Synthesis of 80 & 81

To a solution of 80.1 (130 mg, 0.3271 mmol) in anhydrous THF (2 mL) was added 5-methyl-2H-tetrazole (41.2 mg, 0.4906 mmol) and CH₃COONa (53.6 mg, 0.65 mmol). The mixture was stirred at 45° C. for 16 hours. The reaction mixture was poured into water (20 mL) and the aqueous phase was extracted with EtOAc (3×30 mL). The combined organic phase was washed with brine (2×100 mL), dried over anhydrous Na₂SO₄, filtered, concentrated and purified by flash column (30˜70% of EtOAc in PE) to give 80 (27.3 mg, 20.8%) and 81 (8.9 mg, 6.79%).

80: ¹H NMR (400 MHz, CDCl₃)⁶H 5.50-5.27 (m, 2H), 2.57 (s, 3H), 2.33-2.20 (m, 1H), 2.03-1.63 (m, 9H), 1.58-1.51 (m, 2H), 1.45-1.24 (m, 5H), 1.20 (s, 3H), 1.14-0.71 (m, 10H), 0.63-0.50 (m, 1H). LC-ELSD/MS: purity>99%, MS ESI calcd. for C₂₃H₃₇N₄O₂ [M+H]⁺ 401.3, found 401.3.

81: ¹H NMR (400 MHz, CDCl₃)^{6H} 5.31-5.00 (m, 2H), 2.65 (s, 1H), 2.45 (s, 3H), 2.40-2.28 (m, 1H), 2.06-1.87 (m, 3H), 1.66-1.53 (m, 8H), 1.38-1.24 (m, 5H), 1.20 (s, 3H), 1.03-0.73 (m, 9H), 0.65-0.51 (m, 1H). LC-ELSD/MS: purity>99%, MS ESI calcd. for C₂₃H₃₇N₄O₂ [M+H]⁺ 401.3, found 401.3.

### Example 82 & 83: Synthesis of 1-((3R,5R,8S,9S,10S,13S,14S,17S)-10-ethyl-3-hydroxy-3-methylhexadecahydro-1H-cyclopenta[a]phenanthren-17-yl)-2-(5-methyl-1H-tetrazol-1-yl)ethanone (82) 1-((3R,5R,8S,9S,10S,13S,14S,17S)-10-ethyl-3-hydroxy-3-methylhexadecahydro-1H-cyclopenta[a]phenanthren-17-yl)-2-(5-methyl-2H-tetrazol-2-yl)ethanone (83)

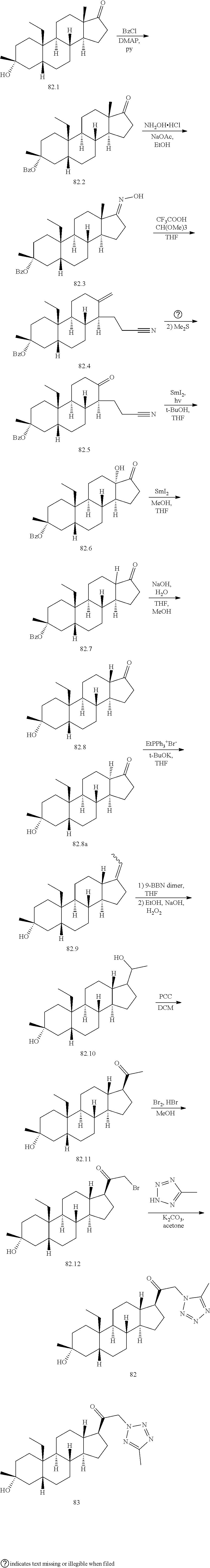

### Synthesis of 82.2

To a solution of 82.1 (20 g, 62.7 mmol) and DMAP (7.66 g, 62.7 mmol) in pyridine (200 mL) was added dropwise benzoyl chloride (14.3 mL, 125 mml) at 20 mC. After stirring at 80° C. for 12 hours to give light yellow solution, the mixture was cooled and then poured into ice-water (200 mL). The aqueous phase was extracted with EtOAc (3×300 mL). The combined organic phase was washed with brine (300 mL), dried over anhydrous Na₂SO₄, filtered and concentrated. The residue was purified by flash column (0˜5% of EtOAc in PE) to give 82.2 (23 g, 83.30). ¹H NMR (400 MHz, CDCl₃) δ_{H} 8.05-7.95 (m, 2H), 7.55-7.35 (m, 3H), 2.48-2.37 (i, 1H), 2.32-2.21 (m, 1H), 2.12-1.69 (in, H), 1.67 (s, 4H), 1.57-1.41 (m, 6H), 1.35-1.14 (i, 5H), 0.90-0.79 (in, 6H).

### Synthesis of 482.3

To a solution of 82.2 (22 g, 52.0 mmol) in EtOH (220 mL) was added hydroxylamine hydrochloride (14.4 g, 208 mmol) and sodium acetate (17.0 g, 208 mmol) at 20° C. under N₂. After refluxing at 80° C. for 16 hours, the mixture was cooled and concentrated to remove most of EtOH. The residue was poured into water (200 mL) and stirred for 20 min. The aqueous phase was extracted with EtOAc (3×150 mL). The combined organic phase was washed with saturated brine (2×50 mL), dried over anhydrous Na₂SO₄, filtered and concentrated. The residue was purified by flash column (0˜20% of EtOAc in PE) to give 82.3 (21 g, 92.5%). ¹H NMR (400 MHz, CDCl₃)^{6H} 8.14-7.95 (m, 2H), 7.54-7.35 (m, 3H), 2.58-2.40 (m, 2H), 2.34-2.20 (m, 1H), 2.03-1.97 (mn, 1H), 1.95-1.70 (m, 7H), 1.67 (s, 3H), 1.65-1.15 (m, 14H), 0.91 (s, 3H), 0.84 (t, J7.6 Hz, 3H).

### Synthesis of 82.4

To a stirred solution of 82.3 (19 g, 43.4 mmol) and CH(OMe)₃ (12.6 g, 119 mmol) in anhydrous THF (200 mL) under N₂ at 60° C. was added freshly distillated TFA (6.18 g, 54.2 mmol) in one portion. After stirring for 2 hours at 65° C., the mixture was quenched with NaHCO₃ (70 mL, sat.). EtOAc (150 mL) was added to the mixture and then filtered. The organic layer was separated, concentrated and purified by flash column (0˜5% EtOAc in PE) to give 82.4 (10 g, 54.9%). ¹H NMR (400 MHz, CDCl₃) δ_{H} 8.08-8.00 (m, 2H), 7.47-7.37 (m, 3H), 4.78 (s, 1H), 4.45 (s, 1H), 2.53-2.21 (m, 5H), 1.94-1.72 (m, 10H), 1.68 (s, 5H), 1.53-1.39 (m, 3H), 1.20-1.04 (m, 3H), 0.85-0.75 (m, 4H).

### Synthesis of 82.5

To a solution of 82.4 (14 g, 33.3 mmol) in DCM (140 mL) and MeOH (140 mL) was added NaHCO₃ (14 g, 166 mmol). The mixture was bubbled with ozone (1 atm) at −70° C. for 30 min until a blue color persisted. The excess ozone was removed by O₂ stream until the mixture turned colorless. Me₂S (14 g, 225 mmol) was added and the mixture was warmed to 20° C. and stirred for 1 h. The mixture was filtered, concentrated and purified by flash column (5˜15% EtOAc in PE) to give 82.5 (9 g, 64.2%). ¹H NMR (400 MHz, CDCl3) δ_{H} 8.06-8.00 (m, 2H), 7.57-7.51 (m, 1H), 7.47-7.39 (m, 2H), 2.51-2.28 (m, 6H), 2.25-2.09 (m, 2H), 2.03-1.73 (m, 7H), 1.70 (s, 3H), 1.67-1.59 (m, 2H), 1.44-1.22 (m, 5H), 1.17-1.08 (m, 1H), 0.82 (t, J=7.2 Hz, 3H).

### Synthesis of 82.6

To a solution of 82.5 (1.5 g, 3.55 mmol) and t-BuOH (526 mg, 7.1 mmol) in THF (35 mL) was added SmI₂ (106 mL, 0.1 M in THF, 10.6 mmol) under N₂ below −20° C. The dark blue mixture was irradiated by two 275 W tungsten lamps for 1 h with being cooled by a flowing coolant to maintaining the inner temperature under −10° C. The mixture was concentrated in vacuum. HCl (10 mL, 2M) was added to the residue and extracted with EtOAc (2×50 mL). The combined organic layer was dried over Na₂SO₄, filtered and concentrated to give 82.6 (1.7 g crude). ¹H NMR (400 MHz, CDCl₃) δ_{H} 8.06-7.98 (m, 2H), 7.57-7.50 (m, 1H), 7.47-7.39 (m, 2H), 2.49-2.35 (m, 3H), 2.34-1.71 (m, 12H), 1.70 (s, 2H), 1.68-1.60 (m, 3H), 1.54-1.52 (m, 1H), 1.48-1.07 (m, 7H), 0.82 (t, J=7.6 Hz, 3H).

### Synthesis of 82.7

To a solution of 82.6 (6.8 g, 16.0 mmol) in THF (150 mL) and MeOH (30 mL) was added SmI₂ (320 mL, 0.1 M in THF, 32.0 mmol) dropwise under N₂ at 10° C. The blue color faded in a few seconds at the beginning till finally obtained a light yellow solution. The mixture was stirred at 10° C. for more than 20 min. The mixture was concentrated in vacuum. HCl (100 mL, 2M) was added to the residue and extracted with EtOAc (2×150 mL). The combined organic layer was dried over Na₂SO₄, filtered, concentrated and purified by flash column (0˜35% EtOAc in PE) to give 82.7 (780 mg, 11.9%). ¹H NMR (400 MHz, CDCl₃) δ_{H} 8.10-7.90 (m, 2H), 7.57-7.49 (m, 1H), 7.46-7.38 (m, 2H), 2.44-2.20 (m, 3H), 2.18-2.06 (m, 2H), 2.01-1.69 (m, 8H), 1.69-1.66 (m, 4H), 1.50-1.40 (m, 4H), 1.16-0.93 (m, 4H), 0.91-0.72 (m, 7H).

### Synthesis of 82.8 & 82.8a

To a solution of 82.7 (780 mg, 1.9 mmol) in THE (5 mL) and MeOH (10 mL) was added a solution of NaOH (759 mg, 19.0 mmol) in water (5 mL). After stirring at 60° C. for 16 hours, the mixture was diluted with water (20 mL) and extracted with EtOAc (2×30 mL). The combined organic layer was dried over Na₂SO₄, filtered, concentrated and purified by flash column (6˜25% EtOAc in PE) to give 82.8a (170 mg, 29.4%) and 82.8 (60 mg, 10.3%).

82.8: ¹H NMR (400 MHz, CDCl₃) δ_{H} 2.42-2.30 (m, 1H), 2.19-1.89 (m, 4H), 1.77-1.58 (m, 7H), 1.54-1.51 (m, 1H), 1.46-1.27 (m, 6H), 1.26 (s, 3H), 1.24-0.97 (m, 5H), 0.80 (t, J=7.6 Hz, 3H). LC-ELSD/MS: purity>99%, MS ESI calcd. for C₂₀H₃₁O [M+H-H₂O]⁺287.2, found 287.2.

### Synthesis of 82.9

To a mixture of EtPPh₃Br (790 mg, 2.13 mmol) in THE (3 mL) was added t-BuOK (239 mg, 2.13 mmol) at 25° C. under N₂. The resulting mixture was stirred at 40° C. for 30 min. 82.8 (130 mg, 0.43 mmol) was added at 40° C. After stirring at 40° C. for 3 hour to give a yellow suspension, the reaction mixture was quenched with saturated NH₄Cl aqueous (50 mL) at 20° C. The aqueous layer was extracted with EtOAc (2×50 mL). The combined organic phase was concentrated. The residue was purified by flash column (0˜20% of EtOAc in PE) to give 82.9 (70 mg, 51.8%). ¹H NMR (400 MHz, CDCl₃) δ_{H} 5.36-4.74 (m, 1H), 2.54-1.59 (m, 13H), 1.50-1.27 (m, 6H), 1.25 (s, 4H), 1.22-0.83 (m, 8H), 0.78 (t, J=7.6 Hz, 3H).

### Synthesis of 82.10

To a solution of 82.9 (70 mg, 0.2211 mmol) in anhydrous THE (2 mL) was added 9-BBN dimer (268 mg, 1.10 mmol) at 25° C. under N₂. The reaction mixture was stirred at 30° C. for 16 hour. To the resulting mixture was added ethanol (506 mg, 11.0 mmol) at 15° C., followed by NaOH aqueous (2.19 mL, 5.0 M, 11.0 mmol) and H₂O₂(1.09 mL, 10 M, 11.0 mmol) was added dropwise at 0° C. After stirring at 60° C. for 1 hour, the mixture was cooled. The mixture was poured into Na₂SO₃ (20 mL, sat. aq.). The aqueous phase was extracted with EtOAc (3×20 mL). The combined organic phase was washed with saturated brine (2×20 mL), dried over anhydrous Na₂SO₄, filtered and concentrated. The residue was purified by flash column (0˜30% of EtOAc in PE) to give 82.10 (50 mg, 67.6%). ¹H NMR (400 MHz, CDCl₃) δ_{H} 3.94-3.59 (m, 2H), 2.5-2.28 (m, 2H), 2.02-1.80 (m, 5H), 1.43-1.28 (m, 8H), 1.25 (s, 7H), 1.21-1.14 (m, 5H), 1.09-0.96 (m, 4H), 0.91-0.85 (m, 2H), 0.77 (t, J=7.2 Hz, 3H).

### Synthesis of 82.11

To a solution of 82.10 (50 mg, 0.1494 mmol) in DCM (5 mL) was added silica gel (70 mg) and PCC (64.3 mg, 0.2988 mmol). After stirring at 25° C. for 1 h, the mixture was concentrated and purified by flash column (0%-15% of EtOAc in PE) to give 82.11 (30 mg, 60.4%). ¹H NMR (400 MHz, CDCl₃) δ_{H} 2.55-2.32 (m, 1H), 2.13 (s, 3H), 2.00-1.66 (m, 8H), 1.49-1.28 (m, 7H), 1.25 (s, 5H), 1.20-0.84 (m, 9H), 0.77 (t, J=7.6 Hz, 3H).

### Synthesis of 82.12

To a solution of 82.11 (30 mg, 0.1 mmol) in MeOH (2 mL) was added HBr (3.62 mg, 0.1 mmol, 40% in water) and Br₂ (14.4 mg, 0.1 mmol) at 25° C. After stirring at 25° C. for 3 hrs, the mixture was poured into NaHCO₃ (5 mL, saturated), extracted with EtOAc (2×10 mL). The organic layer was separated, dried over Na₂SO₄, filtered and concentrated in vacuum to give 82.12 (30 mg, crude). ¹H NMR (400 MHz, CDCl₃) δ_{H} 3.97-3.99 (m, 2H), 2.87-2.70 (m, 1H), 2.01-1.63 (m, 7H), 1.49-1.32 (m, 6H), 1.25 (s, 5H), 1.23-1.04 (m, 8H), 0.92-0.82 (m, 3H), 0.77 (t, J=7.6 Hz, 3H).

### Synthesis of 82 & 83

To a solution of 82.12 (30 mg, 0.073 mmol) in acetone (1 mL) was added 5-methyl-1H-1,2,3,4-tetrazole (24.5 mg, 0.29 mmol), K₂CO₃ (40.3 mg, 0.29 mmol). After stirring at 25° C. for 3 hours, water (5 mL) was added to the mixture and extracted with EtOAc (2×10 mL). The organic layer was separated, dried over Na₂SO₄, filtered and concentrated. The residue was purified by flash column (30-80% of EtOAc in PE) to give 83 (1.0 mg, 3.31%) and 82 (2.1 mg, 6.95%).

83: ¹H NMR (400 MHz, CDCl₃)⁶H 5.45-5.34 (m, 2H), 2.57 (s, 3H), 2.02-1.82 (m, 5H), 1.49-1.34 (m, 7H), 1.25 (s, 8H), 1.20-1.06 (m, 7H), 0.88-0.74 (m, 6H). LC-ELSD/MS: purity>99%, MS ESI calcd. for C₂₄H₃₇N₄O [M−H₂O+H]⁺ 397.3, found 397.3.

82: ¹H NMR (400 MHz, CDCl3)⁶H 5.27-4.99 (m, 2H), 2.69-2.57 (m, 1H), 2.46 (s, 3H), 2.02-1.89 (m, 4H), 1.49-1.34 (m, 7H), 1.28-1.23 (m, 8H), 1.21-1.06 (m, 8H), 0.89-0.82 (m, 2H), 0.81-0.75 (m, 3H). LC-ELSD/MS: purity>99%, MS ESI calcd. for C₂₄H₃₉N₄O₂ [M+H]⁺ 415.3, found 415.3.

### Example 84: Synthesis of 1-(2-((3R,5R,8R,9R,10S,13R,14S,17S)-3-hydroxy-3-methylhexadecahydro-1H-cyclopenta[a]phenanthren-17-yl)-2-oxoethyl)-1H-pyrazole-4-carbonitrile (84)

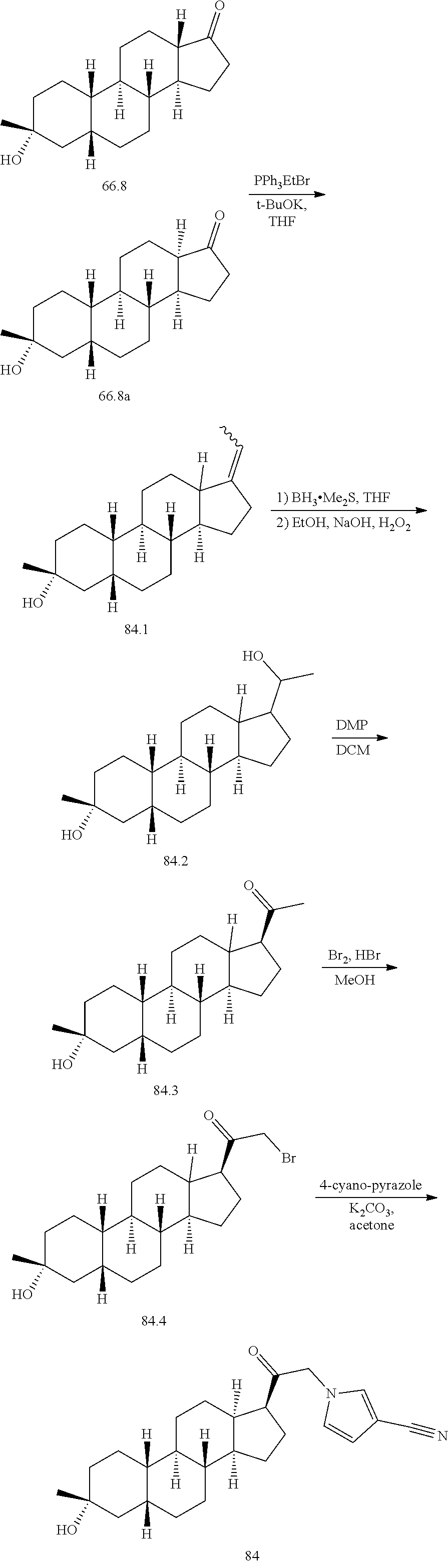

### Synthesis of 84.1

To a suspension of EtPPh₃Br (3.00 g, 8.1 mmol) in THE (12 mL) was added t-BuOK (908 mg, 8.1 mmol) under N₂. The mixture was stirred at 40° C. for 1 h. A solution of 66.8a (450 mg, 1.6 mmol) in THF (3 mL) was added to the mixture. The resulting mixture was stirred at 40° C. for 16 hours to give an orange suspension. The mixture was quenched with NH₄Cl (10 mL, sat.) and extracted with EtOAc (20 mL). The organic layer was separated, dried over Na₂SO₄, filtered, concentrated and purified by flash column (0˜15% EtOAc in PE) to give 84.1 (320 mg, 68%).

### Synthesis of 84.2

To a solution of 84.1 (320 mg, 1.1 mmol) in THE (5 mL) was added BH₃·Me₂S (1.09 mL, 10 M, 10.9 mmol) under N₂. The mixture was stirred at 25° C. for 16 h. EtOH (2.53 g, 55 mmol), NaOH (11 mL, 5M, 55 mmol) and H₂O₂(1.09 mL, 10 M, 10.9 mmol) were added to the mixture. After stirring at 25° C. for 1 h, the mixture was quenched with Na₂SO₃ (20%, 10 mL) and extracted with EtOAc (2×50 mL). The combined organic layer was dried over Na₂SO₄, filtered, concentrated to give 84.2 (300 mg, 89%).

### Synthesis of 84.3

To a solution of 84.2 (300 mg, 0.64 mmol) in DCM (5 mL) was added silica gel (1.2 g) and PCC (419 mg, 1.95 mmol). After stirring at 25° C. for 1 h, the mixture was concentrated and purified by flash column (1st run: 10˜20% EtOAc in PE, 2^{nd} run: 0˜3% acetone in DCM) to give 84.3 (90.0 mg).

### Synthesis of 84.4

To a solution of 84.3 (320 mg, 1.05 mmol) in MeOH (5 mL) was added HBr (42.2 mg, 40% aq., 0.21 mmol) and Br₂ (167 mg, 1.05 mmol). After stirring at 25° C. for 2 h, the mixture was quenched with NaHCO₃ (20 mL, aq., sat.) and extracted with EtOAc (2×20 mL). The combined organic layer was dried over Na₂SO₄, filtered and concentrated to give 84.4 (400 mg, 96%).

### Synthesis of 84

To a solution of 84.4 (400 mg, 1.04 mmol) in acetone (10 mL) was added 4-cyano-pyrazole (145 mg, 1.56 mmol) and K₂CO₃ (291 mg, 2.08 mmol). After stirring at 25° C. for 16 hours, water (20 mL) was added to the mixture and extracted with EtOAc (2×20 mL), The combined organic layer was dried over Na₂SO₄, filtered, concentrated in vacuum, purified by flash column (25-60% EtOAc in PE) to give a mixture of 84 and 68 (300 mg, 1:10). The mixture (400 mg) was separated by SFC (Instrument: SFC-13; Column: DAICEL CHIRALPAK IC (250 mm*30 mm, 5 um); Condition: 0.1% NH₃H₂O ETOH; Begin B: 40%; End B: 40%; FlowRate (mL/min): 65; Injections: 80) two times and re-dissolved in MeCN/water (20 mL/20 mL) and lyophilized to give 84 (19.4 mg). ¹H NMR (400 MHz, CDCl₃)⁶H 7.85 (s, 1H), 7.82 (s, 1H), 5.10 (d, J=18.0 Hz, 1H), 4.93 (d, J=17.6 Hz, 1H), 3.06 (q, 0.1=8.8 Hz, 1H), 2.50-2.40 (m, 1H), 2.15-2.00 (m, 1H), 1.85-1.60 (m, 8H), 1.50-1.20 (m, 15H), 1.15-0.90 (m, 3H). LC-ELSD/MS purity>99%, MS ESI calcd. for C₂₄H₃₂N₃₀ [M+H-H₂O]378.3, found 378.2.

### Example 85: Synthesis of 1-((1S,4aS,4bS,6aR,8R,10aR,10bS,12aS)-10a-cyclopropyl-8-hydroxy-8,12a-dimethyloctadecahydrochrysen-1-yl)ethan-1-one

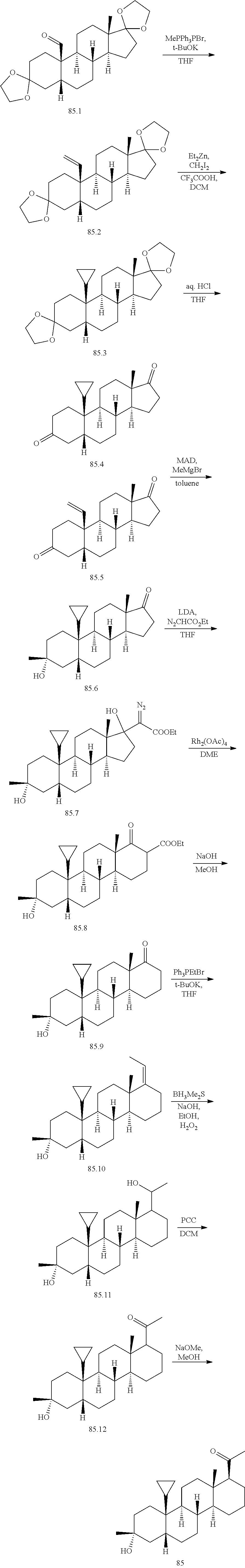

### Synthesis of 85.2

To a suspension of MePPh₃Br (145 g, 408 mmol) in THF (300 mL) was added t-BuOK (45.7 g, 408 mmol) at 15° C. After stirring at 45° C. for 0.5 hour, a solution of 85.1 (CAS #5696-44-6) (80 g, 204 mmol) in THF (200 mL) was added at 45° C. and the reaction mixture was stirred at 45° C. for 1 h. The mixture was diluted with PE (300 mL) then filtered. The filtrate was concentrated to give an oil (200 g, crude). After stirring the crude product with PE (1 L) for 16 hours, the solid was filtered and the filtrate was concentrated to give the impure product 85.2 as an oil, which was used directly in next step. ¹H NMR (400 MHz, CDCl₃) δ_{H} 6.30 (dd, J=11.2, 17.6 Hz, 1H), 5.15-4.96 (m, 2H), 3.94-3.81 (m, 8H), 2.02-1.73 (m, 7H), 1.58-1.35 (m, 13H), 1.22-1.14 (m, 2H), 0.81 (s, 3H).

### Synthesis of 85.3

To a solution of Et₂Zn (30.5 mL, 1M in hexane) in DCM (50 mL) at 0° C. was added dropwise CF₃COOH (3.47 g, 30.4 mmol) (in DCM 5 mL) over a period of 1 h under N₂ atmosphere, then to the mixture was added dropwise CH₂I₂(8.16 g, 30.5 mmol) (in DCM 5 mL) over a period of 15 min. After the addition, 85.2 (4 g, 10.2 mmol) (in DCM 5 mL) was added to the reaction mixture and stirred at 0° C. for 3 hours, then stirred at 25° C. for 16 hours. The reaction was quenched by addition of saturated NH₄Cl aqueous (80 mL), the aqueous phase was extracted with DCM (3×60 mL). The combined organic phase was washed with brine (50 mL), dried over anhydrous Na₂SO₄, filtered and concentrated to give 85.3 (4.5 g) as an oil. (Based on crude H-NMR, there was about 40% starting material remained.) Synthesis of 85.4 & 85.55

To a solution of 85.3 (4.5 g, crude) in THF (25 mL) was added hydrogen chloride (12 mL, 2 M aq.), the reaction mixture was stirred at 25° C. for 16 hours. The reaction mixture was added saturated NaHCO₃ (50 mL) to pH-8 and extracted with EtOAc (2×80 mL). The combined organic phase was washed with brine (50 mL), dried over anhydrous Na₂SO₄, filtered and concentrated. The residue was purified by flash column (0˜10% of EtOAc in PE) to give 85.5 (1.1 g). Purification by SFC (column: DAICEL CHIRALPAK AD (250 mm*50 mm, 10 μm; Condition: 0.1% NH₃H₂O ETOH; Begin B: 25%, End B:25%; FlowRate (ml/min): 200) gave 85.4 (550 mg, 29.1%) and 1.5 (420 mg) both as oils.

85.4: ¹H NMR (400 MHz, CDCl₃) OH 2.73-2.61 (m, 1H), 2.47 (dd, J=8.8, 19.2 Hz, 1H), 2.35-2.22 (m, 1H), 2.17-1.85 (m, 8H), 1.77-1.61 (m, 4H), 1.56-1.50 (m, 1H), 1.47-1.39 (m, 1H), 1.36-1.22 (m, 5H), 0.92 (s, 3H), 0.90-0.84 (m, 1H), 0.54-0.40 (m, 2H), 0.34-0.25 (m, 1H), 0.12-0.04 (m, 1H).

### Synthesis of 85.6

To a solution of BHT (2.22 g, 10.08 mmol) in toluene (6 mL) under nitrogen at 0° C. was added AlMe₃ (2.6 mL, 2 M in toluene) dropwise. The mixture was stirred at 25° C. for 1 h. The reaction mixture was used directly as a solution of MAD for the next step without monitor and further purification. To the MAD (5.04 mmol) solution was added a solution of 85.4 (530 mg, 1.68 mmol) in DCM (5 mL) dropwise at −70° C. After stirring at −70° C. for 1 h under N₂, MeMgBr (1.7 mL, 5.1 mmol, 3M in ethyl ether) was added dropwise at −70° C. The resulting solution was stirred at −70° C. for another 3 hours. The reaction mixture was poured into saturated aqueous citric acid (100 mL) at below 10° C. and extracted with EtOAc (2×80 mL). The combined organic layer was dried over Na₂SO₄, filtered and concentrated. The residue was purified by flash column (0˜20% of EtOAc in PE) to give 85.6 (480 mg, 86.4%) as a solid.

¹H NMR (400 MHz, CDCl₃) δ_{H} 2.44 (dd, J=8.2, 19.2 Hz, 1H), 2.14-1.88 (m, 4H), 1.87-1.76 (m, 2H), 1.64-1.60 (m, 1H), 1.58-1.49 (m, 5H), 1.48-1.33 (m, 4H), 1.31-1.21 (m, 6H), 1.19 (s, 3H), 0.88 (s, 3H), 0.56-0.33 (m, 3H), 0.30-0.20 (m, 1H), 0.10-0.00 (m, 1H). LCMS Rt=0.980 min in 2 min chromatography, 30-90AB-2 min. Lcm. (Mobile Phase: 1.5 mL/4L TFA in water (solvent A) and 0.75 mL/4L TFA in acetonitrile (solvent B), using the elution gradient 30%-90% (solvent B) over 0.9 minutes and holding at 90% for 0.6 minutes at a flow rate of 1.2 mL/min; Column: Xtimate C18 2.1*30 mm, 3 μm; Wavelength: UV 220 nm; Column temperature: 50° C.; MS ionization: ESI; Detector: PDA&ELSD), LC-ELSD purity 100%; MS ESI calcd. for C₂₂H₃₃O [M−H₂O+H]⁺ 313.2, found 313.2.

### Synthesis of 85.7

To a solution of i-Pr₂NH (5.26 g, 52 mmol) in THF (30 mL) under N₂ was added n-BuLi (20.8 mL, 2.5 M, 52 mmol) at −70° C. The mixture was warmed to 0° C. and stirred at 0° C. for 30 min before adding to a solution of 85.6 (4.3 g, 13 mmol) and ethyl diazoacetate (5.93 g, 52 mmol) in THE (400 mL) at −70° C. After stirring at −70° C. for 2 h, acetic acid (3.12 g, 52 mmol) in THF (10 mL) was added, and the reaction mixture warm to 25° C. After stirring for 16 h, the reaction was diluted with water (1000 mL) and PE (300 mL). The organic phase was separated, and the aqueous phase was extracted with EtOAc (500 mL). The combined organic layers were washed with saturated brine (1000 mL), dried over anhydrous Na₂SO₄, filtered and concentrated. The residue was purified by flash column (0˜30% of EtOAc in PE) to give the 85.7 (4 g) as an oil. ¹H NMR (400 MHz, CDCl₃) δ_{H}4.27-4.23 (m, 2H), 2.22-2.05 (m, 2H), 2.02-1.79 (m, 5H), 1.73-1.62 (m, 3H), 1.53-1.47 (m, 6H), 1.46-1.37 (m, 5H), 1.26 (s, 3H), 1.20 (s, 3H), 1.12-1.06 (m, 3H), 0.93 (s, 3H), 0.52-0.34 (m, 3H), 0.28-0.20 (m, 1H), 0.07-0.00 (m, 1H).

### Synthesis of 85.8

To a solution of 85.7 (4 g, 8.99 mmol) in DME (50 mL) was added Rh₂(OAc)₄ (79.4 mg, 0.1798 mmol). After stirring at 25° C. for 16 h, the reaction mixture was concentrated to give 85.8 (4 g) as an oil. ¹H NMR (400 MHz, CDCl₃) δ_{H} 12.42 (s, 1H), 4.60-4.40 (m, 1H), 4.23-4.34 (m, 1H), 3.64-3.74 (m, 1H), 2.25-2.42 (m, 1H), 2.08-2.23 (m, 2H), 1.78-2.04 (m, 6H), 1.60-1.76 (m, 6H), 1.50-1.56 (m, 4H), 1.34-1.44 (m, 4H), 1.28-1.30 (m, 3H), 1.27 (s, 3H), 1.18 (d, J=4.80 Hz, 3H), 0.33-0.51 (m, 3H), 0.19-0.29 (m, 1H), 0.02-0.07 (m, 1H).

### Synthesis of 85.9

To a mixture of 85.8 (4 g, 9.6 mmol) in MeOH (30 mL) was added H₂O (10 mL) and NaOH (3.83 g, 95.9 mmol). After stirring at 60° C. for 16 h, the reaction mixture was concentrated, diluted with H₂O (50 mL) and extracted with EtOAc (2×50 mL). The combined organic phase was washed with saturated brine (50 mL), dried over anhydrous Na₂SO₄, filtered and concentrated to give 85.9 (2 g) as a solid. ¹H NMR (400 MHz, CDCl₃) δ_{H} 2.64-2.56 (m, 1H), 2.23-2.17 (m, 1H), 2.06-1.95 (m, 3H), 1.88-1.82 (m, 2H), 1.78-1.68 (m, 3H), 1.65-1.58 (m, 4H), 1.56-1.47 (m, 8H), 1.41-1.34 (m, 3H), 1.18 (s, 3H), 1.10-1.08 (m, 3H), 1.05-1.03 (m, 1H), 0.50-0.34 (m, 3H), 0.29-0.20 (m, 1H), 0.07-0.02 (m, 1H).

### Synthesis of 85.10

To a solution of MePh₃PBr (12.9 g, 34.8 mmol) in THF (40 mL) was added t-BuOK (3.9 g, 34.8 mmol) at 25° C. After stirring at 60° C. for 1 h, a solution of 85.9 (2 g, 5.8 mmol) in THF (10 mL) was added. After stirring at 60° C. for 16 h, the reaction mixture was added into saturated NH₄Cl (100 mL) and extracted with EtOAc (3×100 mL). The combined organic layer was washed with saturated brine (100 mL), dried over anhydrous Na₂SO₄, filtered and concentrated. The residue was purified by flash column (5-15% of EtOAc in PE) to give 85.10 (2 g, 97%) as a solid. ¹H NMR (400 MHz, CDCl₃) δ_{H} 5.20-5.11 (m, 1H), 2.58-2.09 (m, 2H), 2.01-1.87 (m, 4H), 1.82-1.69 (m, 4H), 1.68-1.56 (m, 8H), 1.55-1.45 (m, 6H), 1.43-1.32 (m, 4H), 1.18-1.16 (m, 3H), 1.07-1.00 (m, 3H), 0.94-0.87 (m, 3H), 0.50-0.32 (m, 3H), 0.29-0.15 (m, 1H), 0.08-0.06 (m, 1H).

### Synthesis of 85.11

To a solution of 85.10 (2 g, 5.6 mmol) in THF (20 ml) was added BH₃·Me₂S (1.67 mL, 16.7 mmol) at 25° C. After stirring at 70° C. for 16 h, the reaction mixture was cooled and quenched by EtOH (3.86 g, 84 mmol, 0.789 g/ml) at 0° C. and then NaOH (16.8 mL, 5M, 84 mmol) very slowly. After addition, H₂O₂(8.4 mL, 84 mmol, 1.13 g/mL, 30% in water) was added slowly until the inner temperature no longer rises and the inner temperature was maintained below 30° C. After stirring at 70° C. for 1 h, the reaction mixture was quenched with saturated aqueous Na₂S₂O₃ (30 mL). After stirring at 0° C. for another 1 h, the aqueous phase was extracted with EtOAc (3×100 mL). The combine organic phase was washed with saturated Na₂S₂O₃ (2×100 mL), brine (2×100 mL), drive over anhydrous Na₂SO₄ filtered and concentrated to give 85.11 (3 g) as a solid. ¹H NMR (400 MHz, CDCl₃) δ_{H} 3.80-3.65 (s, 1H), 2.03-1.78 (m, 5H), 1.64-1.56 (m, 5H), 1.56-1.44 (m, 8H), 1.39-1.26 (m, 5H), 1.24 (s, 3H), 1.18 (s, 3H), 1.17-1.14 (m, 2H), 1.12-1.08 (m, 2H), 0.98-0.91 (m, 3H), 0.87-0.82 (m, 1H), 0.50-0.30 (m, 3H), 0.26-0.16 (m, 1H), 0.05-0.00 (m, 1H).

### Synthesis of 85.12

To a solution of 85.11 (1 g, 2.66 mmol) in DCM (10 mL) was added silica gel (1 g) and PCC (857 mg, 3.99 mmol) at 0° C. After stirring at 25° C. for 1 h, the suspension was filtered, and the filter cake was washed with DCM (2×20 mL). The combined filtrate was concentrated. The residue was purified by flash column (0-30% of EtOAc in PE) to give 85.12 (450 mg, 45.4%) as a solid. ¹H NMR (400 MHz, CDCl₃) δ_{H} 2.53-2.24 (m, 1H), 2.17-2.12 (m, 3H), 1.82-2.03 (m, 3H), 1.64-1.81 (m, 3H), 1.59-1.64 (m, 3H), 1.46-1.57 (m, 7H), 1.28-1.46 (m, 6H), 1.20-1.15 (m, 3H), 0.96-1.12 (m, 2H), 0.94-0.92 (m, 3H), 0.70-0.91 (m, 1H), 0.30-0.49 (m, 3H), 0.17-0.27 (m, 1H), 0.05-0.05 (m, 1H).

### Synthesis of 85

To a solution of 85.12 (200 mg, 0.5367 mmol) in MeOH (50 mL) was added MeONa (289 mg, 5.36 mmol). After stirring at 70° C. for 2 days, the mixture was added to water (50 mL) and extracted with EtOAc (3×30 mL). The combined organic layer was washed with saturated brine (50 mL), dried over anhydrous Na₂SO₄, filtered and concentrated. The residue was purified by flash column (0-30% of EtOAc in PE) to give 85 (100 mg, 50.2%) as a solid. ¹H NMR (400 MHz, CDCl₃) δ_{H} 2.36-2.25 (m, 1H), 2.14 (s, 3H), 2.04-1.78 (m, 3H), 1.77-1.58 (m, 5H), 1.55-1.41 (m, 6H), 1.40-1.20 (m, 8H), 1.19 (s, 3H), 1.18-1.13 (m, 1H), 1.11-0.95 (m, 2H), 0.93 (s, 3H), 0.50-0.30 (m, 3H), 0.28-0.16 (m, 1H), 0.09-0.05 (m, 1H); LC-ELSD/MS: purity >99%; MS ESI calcd. for C₂₅H₃₉O₁[M−H₂O+H]-355.3, found 355.3.

### Example 86 & 87: Synthesis of 1-(2-((1R,4aS,4bS,6aR,8R,10aR,10bS,12aS)-10a-cyclopropyl-8-hydroxy-8,12a-dimethyloctadecahydrochrysen-1-yl)-2-oxoethyl)-1H-pyrazole-4-carbonitrile & 1-(2-((1S,4aS,4bS,6aR,8R,10aR,10bS,12aS)-10a-cyclopropyl-8-hydroxy-8,12a-dimethyloctadecahydrochrysen-1-yl)-2-oxoethyl)-1H-pyrazole-4-carbonitrile

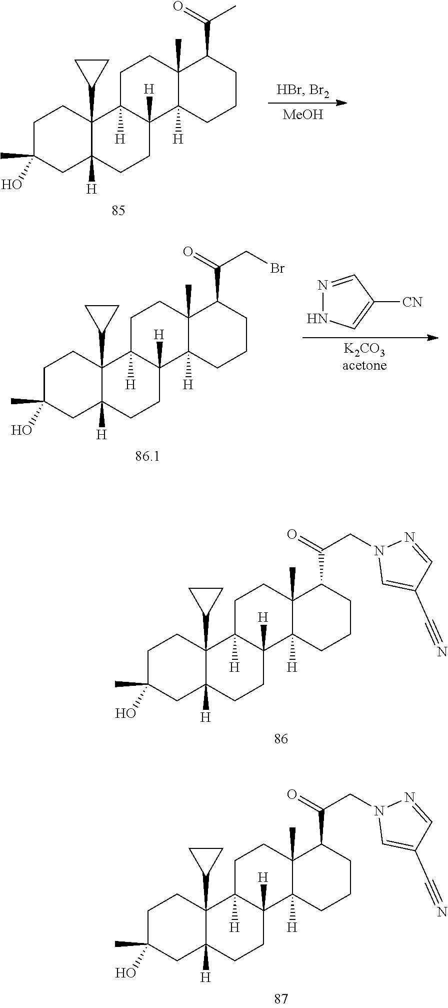

### Synthesis of 86.1

To a solution of 85 (250 mg, 0.6709 mmol) and HBr (26.7 mg, 0.1341 mmol, 40%) in MeOH (10 mL) was added Br₂ (118 mg, 0.7379 mmol) at 0° C. After stirring at 25° C. for 2 h, the reaction mixture was added into saturated NaHCO₃ (50 mL) and extracted with EtOAc (3×20 mL). The combined organic layer was washed with saturated brine (50 mL), dried over anhydrous Na₂SO₄, filtered and concentrated to give 86.1 (300 mg, crude) as an oil. ¹H NMR (400 MHz, CDCl₃)⁶H 5.43-5.45 (m, 1H), 3.91-4.00 (m, 1H), 1.89-2.04 (m, 2H), 1.77-1.89 (m, 2H), 1.65-1.77 (m, 2H), 1.59-1.65 (m, 2H), 1.42-1.58 (m, 7H), 1.28-1.41 (m, 4H), 1.24-1.27 (m, 3H), 1.19-1.23 (m, 2H), 1.18 (d, J=10.40 Hz, 3H), 1.00-1.13 (m, 2H), 0.98-0.94 (m, 3H), 0.75-0.93 (m, 1H), 0.29-0.52 (m, 3H), 0.10-0.28 (m, 1H), 0.06-0.06 (m, 1H).

### Synthesis of 86 & 87

To a solution of 86.1 (300 mg, 0.6644 mmol) in acetone (10 ml) was added K₂CO₃ (182 mg, 1.32 mmol) and 1H-pyrazole-4-carbon (74.2 mg, 0.7972 mmol). After stirring at 25° C. for 2 h, the reaction mixture was poured into saturated water (50 mL), stirred for 10 mins, and extracted with EtOAc (2×50 mL). The organic phase was washed with saturated brine, filtered and concentrated. The residue was purified by flash column (0˜30% of EtOAc in PE) to give 87 (90 mg) and 3 (66.1 mg, 21.4%) as solids. 86 (90 mg, impure) was further purified by HPLC (Column: YMC Triart C18 150*25 mm*5 um; Condition: water (10 mM NH₄HCO₃) ACN; Begin B:78; End B: 100) to afford 86 (14.9 mg, 4.82%).

86: ¹H NMR (400 MHz, CDCl₃)⁶H 7.88 (s, 1H), 7.84 (s, 1H), 5.07-5.01 (m, 1H), 4.93-4.87 (m, 1H), 2.50-2.43 (m, 1H), 2.02-1.85 (m, 3H), 1.84-1.66 (m, 3H), 1.66-1.57 (m, 6H), 1.55-1.49 (m, 2H), 1.49-1.40 (m, 4H), 1.40-1.27 (m, 5H), 1.27-1.18 (m, 5H), 1.16 (s, 3H), 1.09-1.01 (m, 1H), 1.00 (s, 3H), 0.98-0.92 (m, 1H), 0.47-0.34 (m, 3H), 0.27-0.18 (m, 1H), 0.05-0.00 (m, 1H); LC-ELSD/MS: purity >99%; MS ESI calcd. for C₂₉H₄₀N₃O₁[M−H₂O+H]⁻ 446.3, found 446.3.

87: ¹H NMR (400 MHz, CDCl₃)^{6H} 7.82-7.80 (m, 2H) 5.07-4.92 (m, 2H) 2.38-2.29 (m, 1H) 2.00-1.94 (m, 1H) 1.93-1.84 (m, 2H) 1.81-1.71 (m, 2H) 1.68-1.58 (m, 4H) 1.52-1.45 (m, 4H) 1.41-1.34 (m, 3H) 1.33-1.22 (m, 5H) 1.20 (s, 3H) 1.18-1.12 (m, 1H) 1.07-0.99 (m, 2H) 0.96 (s, 3H) 0.92-0.75 (m, 1H) 0.50-0.32 (m, 3H) 0.28-0.19 (m, 1H) 0.06-0.03 (m, 1H); LC-ELSD/MS: purity >99%; MS ESI calcd. for C₂₉H₄₀N₃O₁ [M−H₂O+H]⁺ 446.3, found 446.3.

### Example 88. 1-((3R,5R,8S,9S,10R,13S,14S,17S)-10-cyclopropyl-13-ethyl-3-hydroxy-3-methylhexadecahydro-1H-cyclopenta[a]phenanthren-17-yl)ethanone (88)

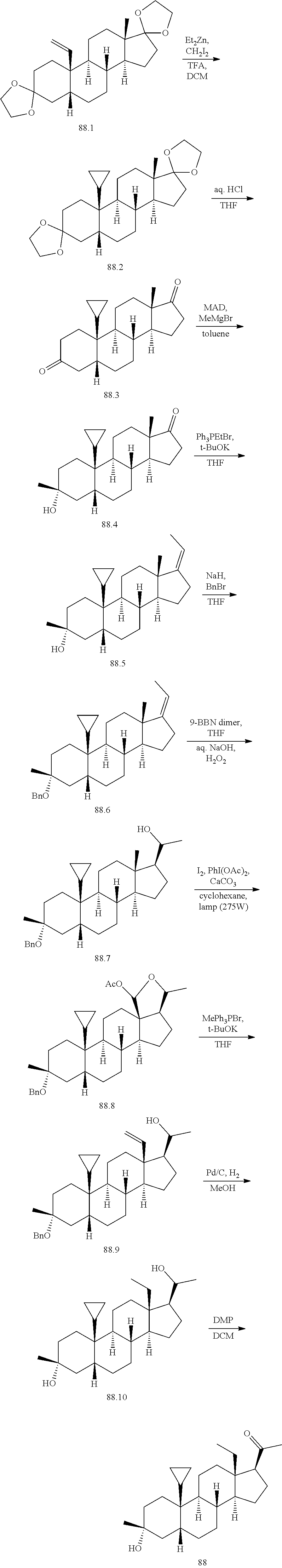

### Synthesis of 88.2

To a solution of Et₂Zn (23.1 mL, 1M in hexane) in DCM (70.0 mL) was added dropwise CF₃COOH (2.20 g, 19.3 mmol) at 0° C. over a period of 0.5 h under N₂ atmosphere. Then, CH₂I₂(6.18 g, 23.1 mmol) was added dropwise to the reaction mixture over a period of 15 min. Finally, the compound 88.1 (3.00 g, 7.72 mmol) in DCM (30.0 mL) was added to the reaction mixture and stirred at 0° C. for 1 hour. After that, the reaction mixture was stirred at 20° C. for 12 hours. The mixture was combined with another two batches (each 3.00 g scale from the compound 88.1) and added into saturated NH₄Cl (300 mL). The aqueous layer was extracted with DCM (2×150 mL). The combined organic layer was washed with saturated brine (300 mL), dried over anhydrous Na₂SO₄, filtered and concentrated to give 88.2 (10.0 g, crude).

### Synthesis of 88.3

To a solution of 88.2 (40.0 g, crude) in THF (300 mL) was added aq. HCl (148 mL, 2.0 M) at 25° C. The mixture was stirred at 25° C. for 2 hours. The mixture was added into saturated brine (200 mL). The aqueous layer was extracted with EtOAc (3×100 mL). The combined organic layer was washed with saturated NaHCO₃ (300 mL), saturated brine (300 mL), dried over anhydrous Na₂SO₄, filtered and concentrated. The residue was purified by flash column (0-15% of EtOAc in PE) to give compound 88.3 (10.0 g, 31.8%). ¹H NMR (400 MHz, CDCl₃)⁶H 2.78-2.58 (m, 1H), 2.52-2.39 (m, 1H), 2.35-2.23 (m, 1H), 2.18-2.05 (m, 4H), 2.04-1.61 (m, 9H), 1.57-1.27 (m, 6H), 0.95-0.83 (m, 4H), 0.59-0.24 (m, 4H).

### Synthesis of 88.4

To the fresh prepared MAD (95.1 mmol) solution in toluene (200 mL) was added the compound 88.3 (10.0 g, 31.7 mmol) in DCM (50 mL) dropwise at −70° C. After stirring at −70° C. for 1 h under N₂, CH₃MgBr (31.7 mL, 95.1 mmol, 3M in ethyl ether) was added dropwise at −70° C. The resulting solution was stirred at −70° C. for another 4 h. The reaction mixture was poured into saturated aqueous citric acid (400 mL, 10%) at below 10° C. and extracted with EtOAc (2×200 mL). The combined organic layer was dried over Na₂SO₄, filtered and concentrated. The residue was purified by flash column (0-30% of EtOAc in PE) to give compound 88.4 (6.90 g, 66.3%). ¹H NMR (400 MHz, CDCl3) δ_{H} 2.50-2.38 (m, 1H), 2.15-1.75 (m, 7H), 1.55-1.22 (m, 15H), 1.19 (s, 3H), 0.91-0.82 (m, 3H), 0.54-0.00 (m, 5H).

### Synthesis of 88.5

To a mixture of EtPPh₃Br (23.1 g, 62.4 mmol) in THE (100 mL) was added t-BuOK (6.98 g, 62.4 mmol) at 15° C. under N₂. The resulting mixture was stirred at 40° C. for 60 min. The compound 88.4 (6.90 g, 20.8 mmol) was added in portions at 50° C. The reaction mixture was stirred at 65° C. for 16 hour to give an orange suspension. The reaction mixture was quenched with aqueous NH₄Cl (200 mL, 10%) at 15° C. THE layer was separated. The aqueous was extracted with EtOAc (2×200 mL). The combined organic phase was concentrated under vacuum to give a white solid, which was purified by flash column (0˜10% of EtOAc in PE) to give compound 88.5 (6.50 g, 91.2%). ¹H NMR (400 MHz, CDCl₃) δ_{H} 5.10-4.99 (m, 1H), 2.41-2.12 (m, 4H), 2.04-1.81 (m, 3H), 1.66-1.61 (m, 6H), 1.53-1.46 (m, 5H), 1.40-1.28 (m, 4H), 1.26-1.22 (m, 4H), 1.19-1.18 (m, 3H), 0.89 (s, 3H), 0.51-0.33 (m, 3H), 0.28-0.17 (m, 1H), 0.07-0.03 (m, 1H).

### Synthesis of 88.6

To a mixture of 88.5 (6.50 g, 18.9 mmol) in DMF (70 mL) was added NaH (3.01 g, 75.6 mmol, 60%) at 0° C. The mixture was stirred at 25° C. for 1 h. Then BnBr (8.95 mL, 75.6 mmol) was added into the mixture at 25° C. The reaction mixture was stirred at 60° C. for 20 hours. Triethylamine (30.0 mL) was added and the mixture was stirred at 60° C. for another 30 min. The mixture was added into aq. NH₄Cl (240 mL, 1M). The aqueous phase was extracted with EtOAc (3×125 mL). The combined organic phase was washed with saturated brine (2×125 mL), dried over anhydrous Na₂SO₄, filtered and concentrated. The residue was purified by flash column (0-0.5% of EtOAc in PE) to give compound 88.6 (5.30 g, impure).

¹H NMR (400 MHz, CDCl₃) δ_{H} 7.24-7.20 (m, 5H), 5.02-4.98 (m, 1H), 4.40-4.36 (m, 2H), 2.29-1.73 (m, 7H), 1.53-1.32 (m, 14H), 1.19-1.11 (m, 7H), 0.78 (s, 3H), 0.44-0.23 (m, 3H), 0.17-0.07 (m, 1H), −0.01-0.12 (m, 1H).

### Synthesis of 88.7

To a solution of 88.6 (5.30 g, 12.2 mmol) in THF (150 mL) was added 9-BBN dimer (14.7 g, 61.0 mmol) and the mixture was stirred at 15° C. for 16 h. To the mixture was added EtOH (21 mL) dropwise followed by NaOH (14.6 g in 73.2 mL water, 5 M) and H₂O₂ (36.6 mL, 10 M, 366 mmol). The mixture was stirred at 78° C. for 2 h. The mixture was quenched by aq. Na₂S₂SO₃ (200 mL, 10%) and extracted with EtOAc (2×100 mL). The organic layer was separated, dried over Na₂SO₄, filtered and concentrated. The residue was purified by flash column (0-10% of EtOAc in PE) to give 88.7 (2.80 g, mixture). ¹H NMR (400 MHz, CDCl₃) δ_{H} 7.40-7.28 (m, 4H), 7.26-7.21 (m, 1H), 4.48 (s, 2H), 3.75-3.65 (m, 1H), 2.04-1.81 (m, 4H), 1.70-1.62 (m, 2H), 1.58-1.31 (m, 10H), 1.30-1.07 (m, 14H), 0.67 (s, 3H), 0.55-0.44 (m, 1H), 0.43-0.33 (m, 2H), 0.27-0.18 (m, 1H), 0.09-0.01 (m, 1H).

### Synthesis of 88.8

To a solution of 88.7 (600 mg, 1.33 mmol) in cyclohexane (60 mL) were added CaCO₃ (399 mg, 3.99 mmol), PhI(OAc)₂ (1.28 g, 3.99 mmol), I₂ (675 mg, 2.66 mmol) and at 25° C. under N₂. The mixture was heated to reflux (80° C.) by irradiated with infrared lamp (250 W) for 15 min. The reaction mixture was cooled to 25° C. and quenched with aqueous Na₂S₂O₃ (60.0 mL). The aqueous layer was extracted with EtOAc (2×30 mL). The combined organic layer was washed with brine (60.0 mL), dried Na₂SO₄ and concentrated. The mixture was filtered and concentrated to give 88.8 (673 mg, crude) as orange oil, which was used directly without further purification.

### Synthesis of 88.9

To a mixture of MePPh₃Br (11.9 g, 33.4 mmol) in THE (50.0 mL) was added t-BuOK (3.74 g, 33.4 mmol) at 25° C. under N₂. The resulting mixture was stirred at 50° C. for 1 h. The compound 88.8 (2.12 g, 4.18 mmol) in THF (10 mL) was added in portions at 50° C. to the above mixture. The reaction mixture was stirred at 50° C. for 12 hour. The reaction mixture was poured into water (150 mL) at 25° C. The aqueous phase was extracted with EtOAc (2×50 mL) and the organic layer was washed with water (50.0 mL), brine (100 mL), dried over Na₂SO₄, filtered and concentrated in vacuum. The residue was purified by silica gel chromatography (0-5% EtOAc in PE) to give 88.9 (800 mg, 41.4%). ¹H NMR (400 MHz, CDCl₃)^{6H} 7.40-7.29 (m, 4H), 7.26-7.17 (m, 1H), 5.89-5.67 (m, 1H), 5.35-5.09 (m, 2H), 4.48 (s, 2H), 3.90-3.74 (m, 1H), 2.44-2.30 (m, 1H), 2.03-1.91 (m, 2H), 1.86-1.60 (m, 6H), 1.57-1.26 (m, 11H), 1.25 (s, 3H), 1.23-1.04 (m, 7H), 0.52-0.40 (m, 1H), 0.37-0.26 (m, 2H), 0.25-0.14 (m, 1H), 0.05-0.05 (m, 1H).

### Synthesis of 88.10

To a solution of 88.9 (800 mg, 1.72 mmol) in MeOH (40 mL) was added Pd/C (160 mg, 10% Palladium on carbon, 50% water wet). Then the solution was hydrogenated under 30 psi of hydrogen at 25° C. for 24 h. The reaction mixture was filtered through a pad of Celite and washed with MeOH (3×20 mL). The filtrate was concentrated in vacuum and the residue was purified by silica gel chromatography (0-40% EtOAc in PE) to give 88.10 (590 mg, crude). ¹H NMR (400 MHz, CDCl₃) δ_{H} 3.94-3.76 (m, 1H), 2.19-2.10 (m, 1H), 2.05-1.83 (m, 3H), 1.75-1.59 (m, 3H), 1.57-1.29 (m, 11H), 1.28-1.20 (m, 9H), 1.19 (s, 3H), 1.17-1.09 (m, 2H), 0.97-0.92 (m, 1H), 0.88-0.81 (m, 3H), 0.52-0.41 (m, 1H), 0.40-0.30 (m, 2H), 0.27-0.17 (m, 1H), 0.08-0.03 (m, 1H).

### Synthesis of 88.

To a solution of 88.10 (590 mg, 1.57 mmol) in DCM (21 mL) was added DMP (1.33 g, 3.14 mmol). The reaction mixture was stirredat 40° C. for 10 min to give a brown solution. The mixture was quenched by saturated NaHCO₃ aqueous (50 mL) at 10° C. The DCM phase was separated and washed with saturated NaHCO₃/Na₂S₂O₃ aqueous (1:1, 2×50 mL), brine (50 mL), dried over anhydrous Na₂SO₄, filtered and concentrated to give the crude product. The crude product was purified by silica gel chromatography (0-10% of EtOAc in PE) to give the compound 88 (450 mg, 77.0%). ¹H NMR (400 MHz, CDCl₃) δ_{H} 2.51-2.41 (m, 1H), 2.34-2.25 (m, 1H), 2.20 (s, 3H), 2.06-1.88 (m, 2H), 1.80-1.57 (m, 4H), 1.57-1.31 (m, 10H), 1.30-1.13 (m, 11H), 0.63 (t, J=7.2 Hz, 3H), 0.53-0.43 (m, 1H), 0.41-0.31 (m, 2H), 0.28-0.18 (m, 1H), 0.09-0.03 (m, 1H).

### Example 89: Synthesis of 1-(2-((3R,5R,8S,9S,10R,13S,14S,17S)-10-cyclopropyl-13-ethyl-3-hydroxy-3-methylhexadecahydro-1H-cyclopenta[a]phenanthren-17-yl)-2-oxoethyl)-1H-pyrazole-4-carbonitrile (89)

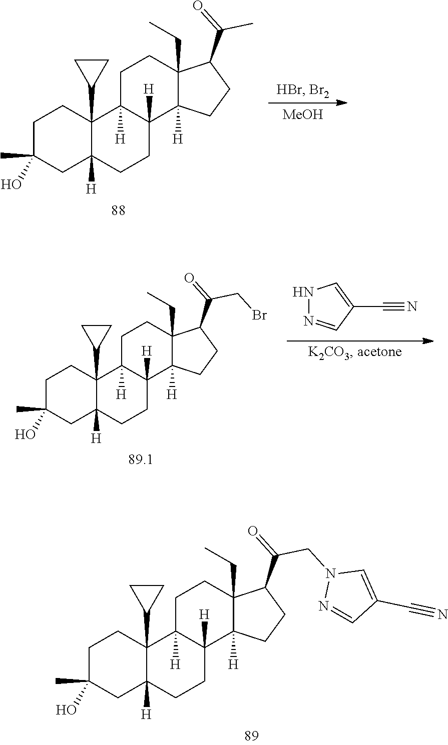

### Synthesis of 89.1

To a solution of 89 (450 mg, 1.20 mmol) in methanol (10 mL) was added HBr (47.9 mg, 240 μmol, 40%) and Br₂ (210 mg, 1.32 mmol) dropwise at 25° C. and stirred for 1 h. To the reaction mixture was added aq. NaHCO₃ (30 mL, 1 M) at 25° C. The aqueous phase was extracted with EtOAc (2×30 mL). The combined organic phase was washed with saturated brine (2×50 mL), dried over anhydrous Na₂SO₄, filtered and concentrated in vacuum to give compound 89.1 (540 mg, crude).

### Synthesis of 89

To a solution of 89.1 (180 mg, 0.398 mmol) in acetone (4 mL) was added 4-cyanopyrazole (44.4 mg, 0.477 mmol) and K₂CO₃(109 mg, 0.796 mmol). The mixture was stirred at 25° C. for 16 h. To the mixture was added water (20 mL) and the mixture was extracted with EtOAc (2×20 mL). The organic layer was separated, concentrated and purified by flash column (0˜35% EtOAc in PE) to give the compound 89 (100 mg, 54.3%).

¹H NMR (400 MHz, CDCl₃) δ_{H} 7.86 (s, 1H), 7.81 (s, 1H), 5.21-4.88 (m, 2H), 2.54-2.45 (m, 1H), 2.39-2.23 (m, 2H), 2.07-1.88 (m, 2H), 1.81-1.57 (m, 5H), 1.54-1.34 (m, 8H), 1.32-1.17 (m, 11H), 0.61 (t, J=7.2 Hz, 3H), 0.55-0.45 (m, 1H), 0.41-0.31 (m, 2H), 0.29-0.19 (m, 1H), 0.10-0.02 (m, 1H). LC-ELSD/MS purity>99%, MS ESI calcd. for C₂₉H₄₁N₃O₂ [M+H-H₂O]⁺ 446.3, found 446.3.

### Example 90: 1-(2-((3R,5R,8S,9S,10R,13S,14S,17S)-10-cyclopropyl-13-ethyl-3-hydroxy-3-methylhexadecahydro-1H-cyclopenta[a]phenanthren-17-yl)-2-oxoethyl)-1H-pyrazole-3-carbonitrile (90)

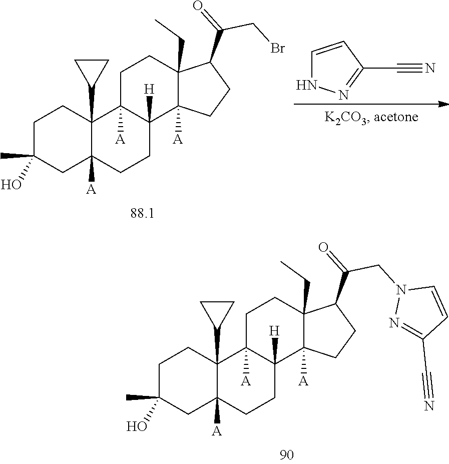

To a solution of 88.1 (100 mg, 0.221 mmol) in acetone (4.0 mL) was added 3-cyanopyrazole (24.6 mg, 0.265 mmol) and K₂CO₃ (60.9 mg, 0.442 mmol). The mixture was stirred at 25° C. for 1 h. To the mixture was added water (20 mL) and the mixture was extracted with EtOAc (2×20 mL). The organic layer was separated, concentrated and purified by flash column (0˜35% EtOAc in PE) to give compound 90 (35.0 mg, 34.3%). ¹H NMR (400 MHz, CDCl₃)^{6H} 7.56-7.44 (m, 1H), 6.81-6.63 (m, 1H), 5.28-4.81 (m, 2H), 2.57-2.43 (m, 1H), 2.41-2.23 (m, 2H), 2.09-1.88 (m, 2H), 1.81-1.61 (m, 4H), 1.55-1.35 (m, 8H), 1.33-1.22 (m, 8H), 1.20 (s, 3H), 1.19-1.13 (m, 1H), 0.62 (t, J=7.6 Hz, 3H), 0.55-0.44 (m, 1H), 0.43-0.31 (m, 2H), 0.30-0.19 (m, 1H), 0.06-0.00 (m, 1H). LC-ELSD/MS purity>99%, MS ESI calcd. for C₂₉H₄₁N₃O₂ [M+H-H₂O]⁺446.3, found 446.3.

### Example 91 & 92. 1-((3R,5R,8S,9S,10R,13S,14S,17S)-10-cyclopropyl-13-ethyl-3-hydroxy-3-methylhexadecahydro-1H-cyclopenta[a]phenanthren-17-yl)-2-(5-methyl-2H-tetrazol-2-yl)ethanone (91) & 1-((3R,5R,8S,9S,10R,13S,14S,17S)-10-cyclopropyl-13-ethyl-3-hydroxy-3-methylhexadecahydro-1H-cyclopenta[a]phenanthren-17-yl)-2-(5-methyl-1H-tetrazol-1-yl)ethanone (92)

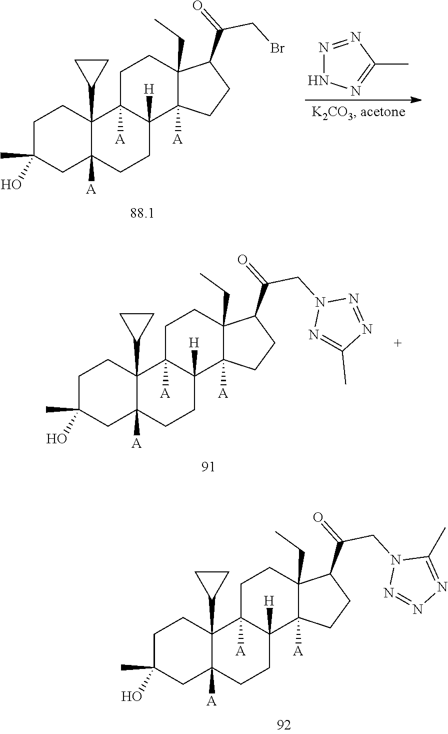

To a solution of 88.1 (260 mg, 0.575 mmol) in acetone (6.0 mL) was added 5-methyl-2H-1,2,3,4-tetrazole (58.0 mg, 0.690 mmol) and K₂CO₃ (158 mg, 1.15 mmol). The mixture was stirred at 25° C. for 16 h. To the mixture was added water (20 mL) and the mixture was extracted with EtOAc (2×20 mL). The organic layer was separated, concentrated and purified by flash column (0˜35˜70% EtOAc in PE) to give the compound 91 (35.0 mg, 13.4%) and the compound 92 (85.0 mg, 32.5%). The structures of these two compounds were assigned based on H-NMR.

Compound 91: ¹H NMR (400 MHz, CDCl₃) δ_{H} 5.55-5.30 (m, 2H), 2.57 (s, 3H), 2.55-2.49 (m, 1H), 2.41-2.24 (m, 2H), 2.07-1.89 (m, 2H), 1.82-1.58 (m, 5H), 1.54-1.33 (m, 8H), 1.32-1.22 (m, 7H), 1.20 (s, 3H), 1.19-1.13 (m, 1H), 0.72 (t, J=7.2 Hz, 3H), 0.56-0.45 (m, 1H), 0.43-0.33 (m, 2H), 0.30-0.20 (m, 1H), 0.09-0.00 (m, 1H). LC-ELSD/MS purity>99%, MS ESI calcd. for C₂₇H₄₂N₄O₂ [M+H-H₂O]⁺ 437.3, found 437.3.

Compound 92: ¹H NMR (400 MHz, CDCl₃) δ_{H} 5.49-4.82 (m, 2H), 2.58-2.52 (m, 1H), 2.49 (s, 3H), 2.44-2.37 (m, 1H), 2.34-2.23 (m, 1H), 2.10-1.88 (m, 2H), 1.84-1.60 (m, 5H), 1.55-1.33 (m, 9H), 1.32-1.23 (m, 6H), 1.21 (s, 3H), 1.19-1.14 (m, 1H), 0.64 (t, J=7.6 Hz, 3H), 0.57-0.45 (m, 1H), 0.43-0.32 (m, 2H), 0.30-0.19 (m, 1H), 0.11-0.01 (m, 1H). LC-ELSD/MS purity>99%, MS ESI calcd. for C₂₇H₄₂N₄O₂ [M+H-H₂O]⁺ 437.3, found 437.3.

### Steroid Inhibition of TBPS Binding

[35S]-t-Butylbicyclophosphorothionate (TBPS) binding assays using rat brain cortical membranes in the presence of 5 mM GABA has been described (Gee et al, J. Pharmacol. Exp. Ther. 1987, 241, 346-353; Hawkinson et al, Mol. Pharmacol. 1994, 46, 977-985; Lewin, A. H et al., Mol. Pharmacol. 1989, 35, 189-194).

Briefly, cortices are rapidly removed following decapitation of carbon dioxide-anesthetized Sprague-Dawley rats (200-250 g). The cortices are homogenized in 10 volumes of ice-cold 0.32 M sucrose using a glass/teflon homogenizer and centrifuged at 1500×g for 10 min at 4° C. The resultant supernatants are centrifuged at 10,000×g for 20 min at 4° C. to obtain the P2 pellets. The P2 pellets are resuspended in 200 mM NaCl/50 mM Na—K phosphate pH 7.4 buffer and centrifuged at 10,000×g for 10 min at 4° C. This washing procedure is repeated twice and the pellets are resuspended in 10 volumes of buffer. Aliquots (100 mL) of the membrane suspensions are incubated with 3 nM [³⁵S]-TBPS and 5 mL aliquots of test drug dissolved in dimethyl sulfoxide (DMSO) (final 0.5%) in the presence of 5 mM GABA. The incubation is brought to a final volume of 1.0 mL with buffer. Nonspecific binding is determined in the presence of 2 mM unlabeled TBPS and ranged from 15 to 25%. Following a 90 min incubation at room temp, the assays are terminated by filtration through glass fiber filters (Schleicher and Schuell No. 32) using a cell harvester (Brandel) and rinsed three times with ice-cold buffer. Filter bound radioactivity is measured by liquid scintillation spectrometry. Non-linear curve fitting of the overall data for each drug averaged for each concentration is done using Prism (GraphPad). The data are fit to a partial instead of a full inhibition model if the sum of squares is significantly lower by F-test. Similarly, the data are fit to a two component instead of a one component inhibition model if the sum of squares is significantly lower by F-test. The concentration of test compound producing 50% inhibition (IC₅₀) of specific binding and the maximal extent of inhibition (Iₘₐₓ) are determined for the individual experiments with the same model used for the overall data and then the means±SEM.s of the individual experiments are calculated. Picrotoxin serves as the positive control for these studies as it has been demonstrated to robustly inhibit TBPS binding.

Various compounds are or can be screened to determine their potential as modulators of [³⁵S]-TBPS binding in vitro. These assays are or can be performed in accordance with the above

In Table 4-6 below, A indicates a TBPS IC₅₀<0.1 μM, B indicates a TBPS IC₅₀ (μM) of 0.1 μM to <1.0 μM, C indicates a TBPS IC₅₀ (μM) of >1.0 μM.

TABLE 4

| Example | STRUCTURE /ID | IC₅₀ |
|---|---|---|
| 1 | 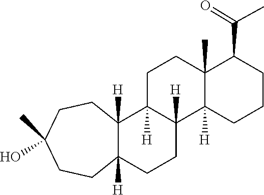 | C |
| 3 | 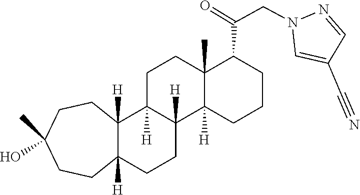 | C |
| 4 | 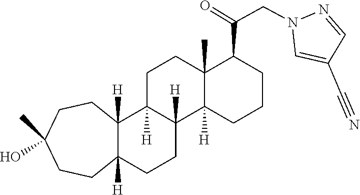 | B |
| 9 | 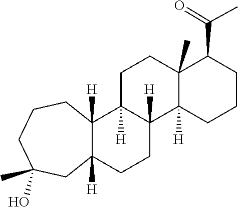 | A |
| 10 | 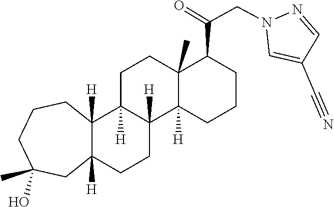 | A |
| 11 | 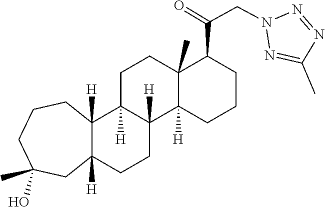 | A |
| 12 | 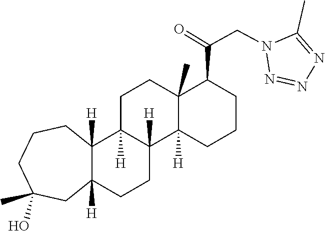 | A |
| 13 | 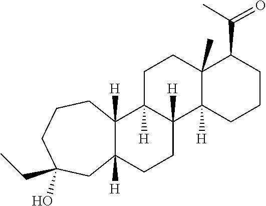 | B |
| 14 | 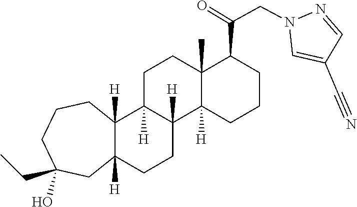 | B |
| 15 | 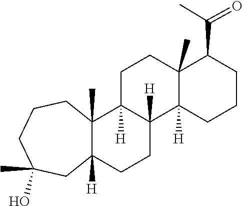 | A |
| 16 | 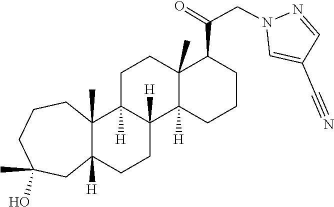 | A |
| 17 | 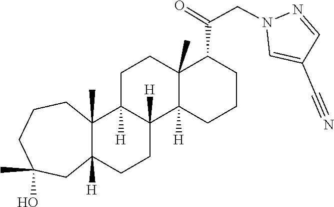 | C |
| 18 | 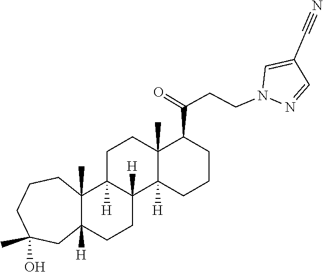 | A |
| 19 | 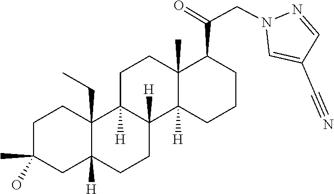 | A |
| 20 | 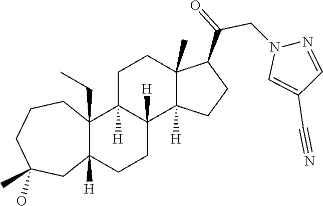 | A |
| 21 | 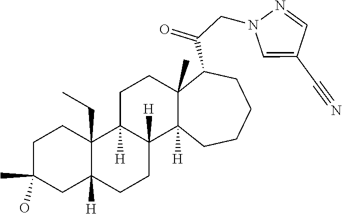 | B |
| 22 | 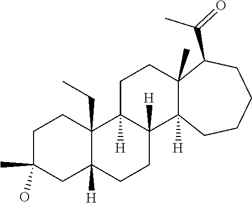 | A |
| 23 | 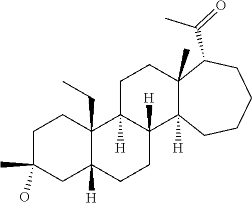 | B |
| 24 | 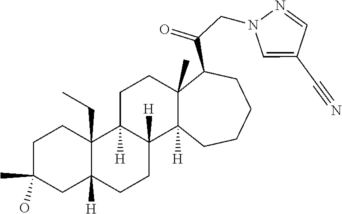 | A |
| 25 | 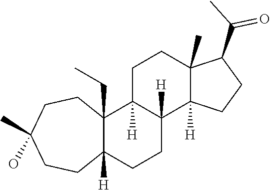 | B |
| 26 | 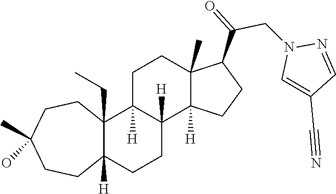 | B |
| 27 | 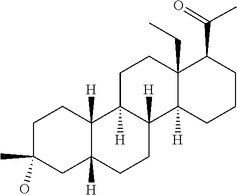 | A |
| 28 | 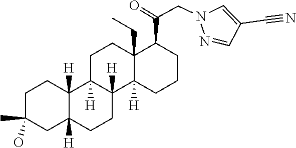 | A |
| 29 | 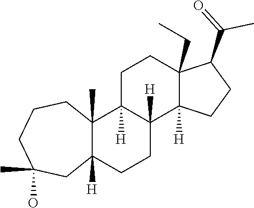 | A |
| 30 | 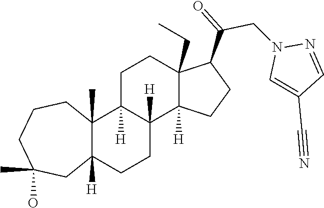 | A |
| 31 | 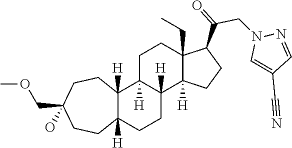 | B |
| 32 | 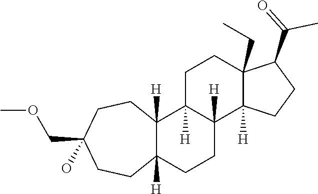 | B |
| 33 | 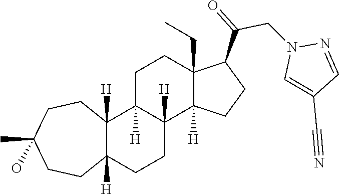 | B |
| 34 | 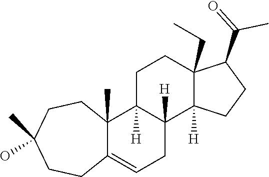 | A |
| 35 | 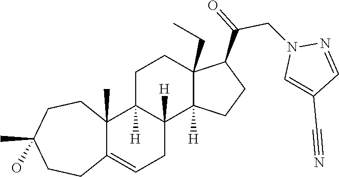 | A |
| 36 | 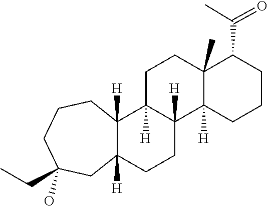 | C |
| 37 | 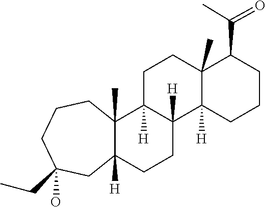 | B |
| 38 | 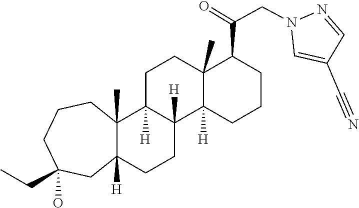 | B |
| 39 | 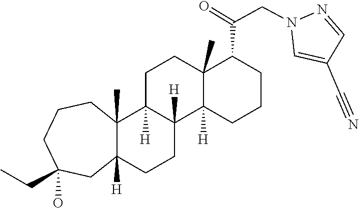 | C |
| 40 | 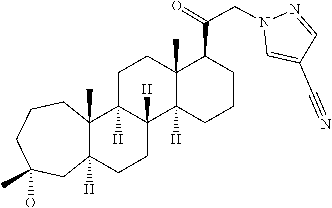 | A |
| 41 | 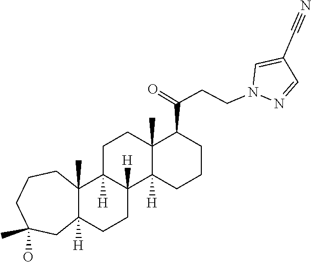 | A |
| 42 | 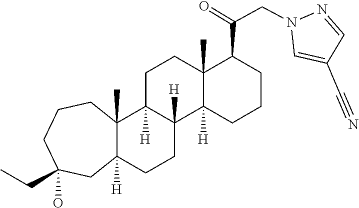 | B |
| 43 | 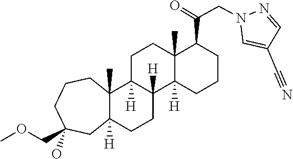 | A |
| 44 | 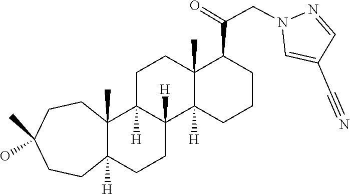 | B |
| 45 | 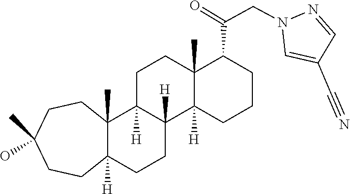 | C |
| 46 | 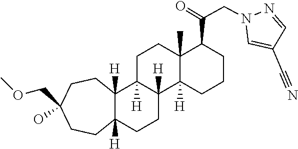 | B |
| 47 | 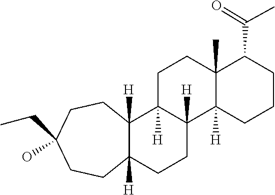 | C |
| 48 | 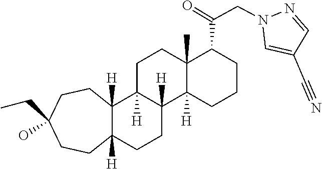 | C |
| 49 | 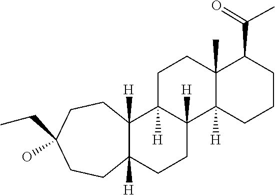 | C |
| 50 | 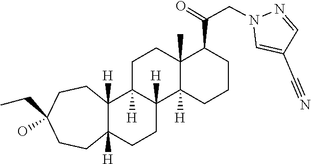 | B |
| 51 | 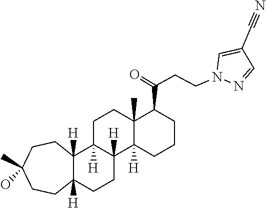 | B |
| 52 | 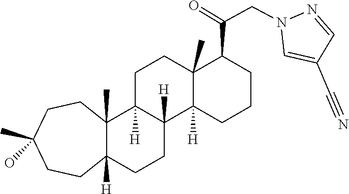 | B |
| 53 | 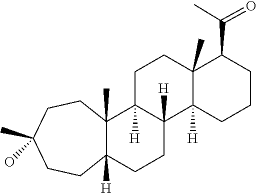 | C |
| 54 | 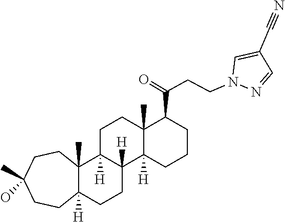 | B |
| 55 | 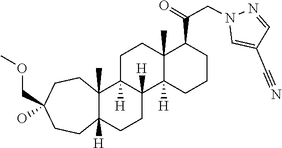 | B |
| 56 | 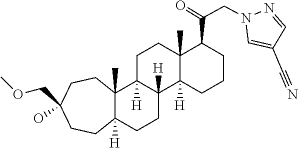 | B |
| 57 | 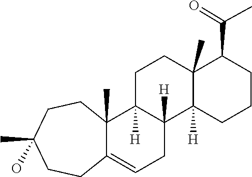 | A |
| 58 | 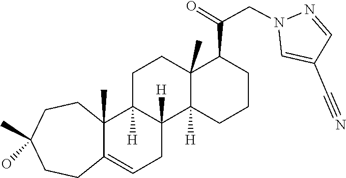 | A |
| 59 | 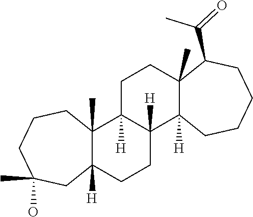 | B |
| 60 | 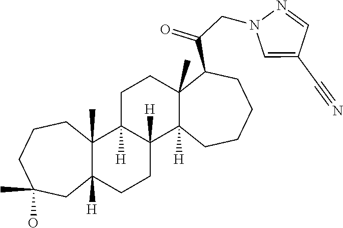 | A |
| 61 | 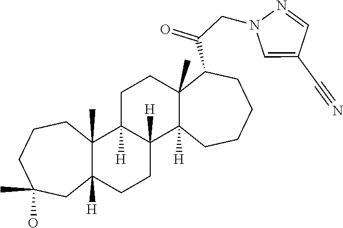 | B |
| 62 | 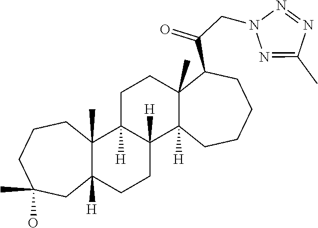 | A |
| 63 | 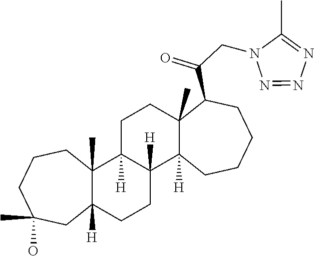 | B |
| 64 | 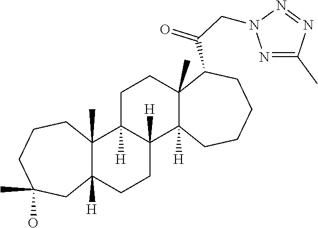 | B |
| 65 | 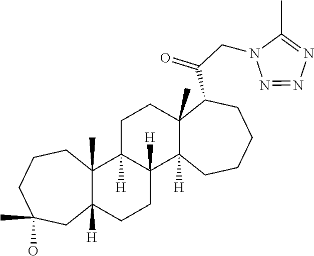 | C |

TABLE 5

| | | |
|---|---|---|
| 66 | 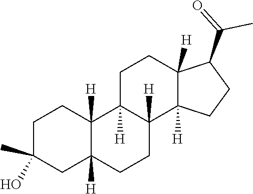 | A |
| 67 | 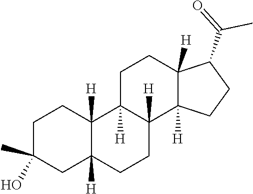 | C |
| 68 | 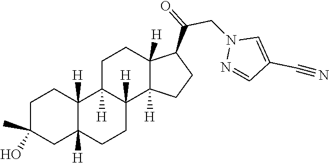 | A |
| 69 | 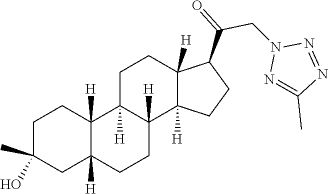 | A |
| 70 | 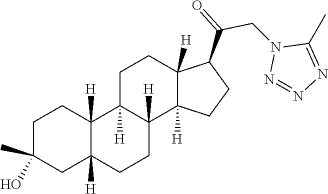 | A |
| 71 | 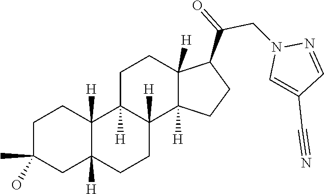 | C |
| 72 | 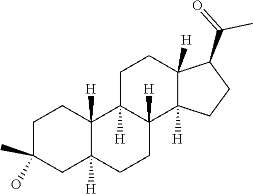 | C |
| 73 | 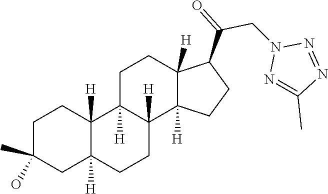 | C |
| 74 | 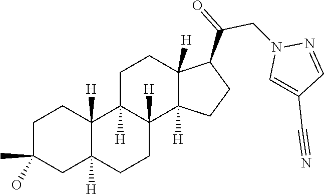 | B |
| 75 | 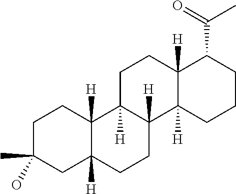 | C |
| 76 | 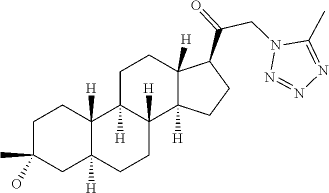 | C |
| 77 | 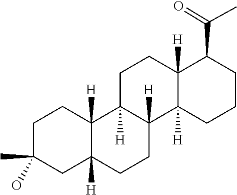 | A |
| 78 | 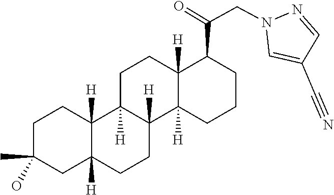 | A |
| 79 | 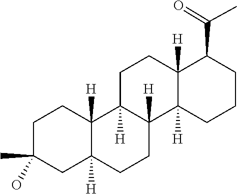 | B |
| 80 | 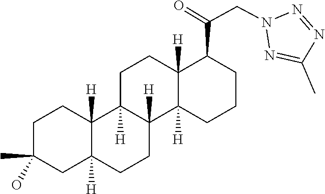 | A |
| 81 | 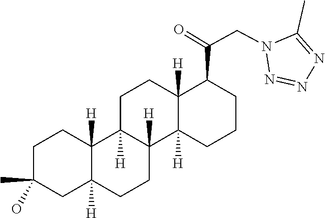 | A |
| 82 | 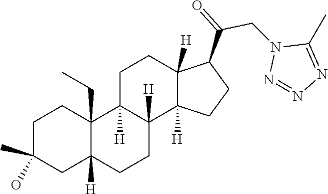 | A |
| 83 | 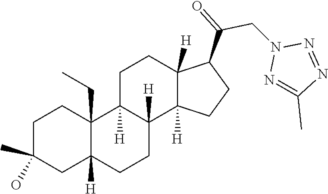 | A |
| 84 | 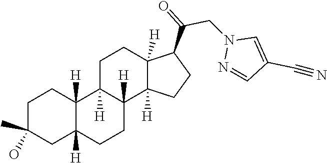 | B |

TABLE 6

| | | IC50 |
|---|---|---|
| Example | Structure | (uM) |
| 85 | 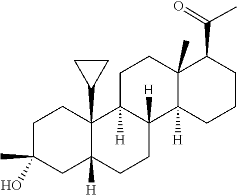 | A |
| 86 | 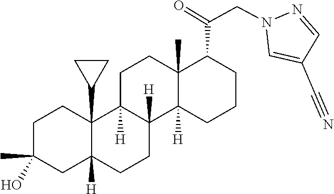 | C |
| 87 | 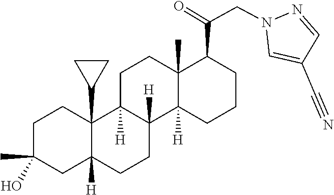 | A |
| 88 | 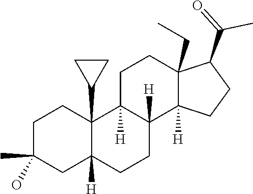 | A |
| 89 | 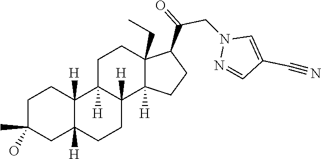 | A |
| 90 | 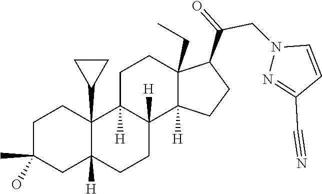 | A |
| 91 | 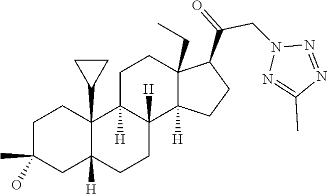 | A |
| 92 | 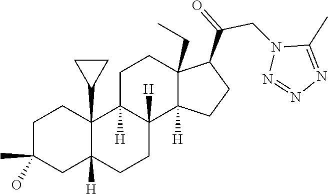 | A |

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1-54. (canceled)

55. A compound of Formula (1-III-a), (1-III-b), (1-III-c), or (1-III-d): 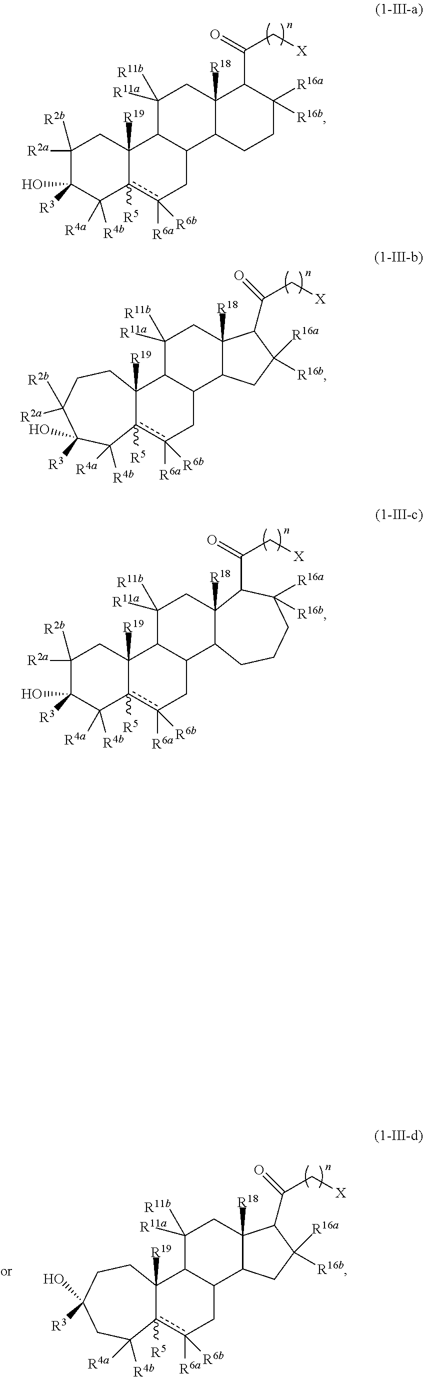
or a pharmaceutically acceptable salt thereof,
wherein: [symbol]
is a single or double bond, provided if a double bond is present, then R⁵ and one of R^{6a} or R^{6b} are absent;
each n is independently 1 or 2;
each X is independently hydrogen, halogen, or 5-6 membered monocyclic heteroaryl having 2-4 nitrogen atoms, wherein the heteroaryl is optionally substituted with one group selected from halo, C₁₋₃ alkyl, —CN, or C₁₋₃ alkoxy;
R⁵ is hydrogen or methyl, or when [symbol] is a double bond, R⁵ is absent;
R¹⁹ is hydrogen, methyl, ethyl, or unsubstituted cyclopropyl;
R¹⁸ is methyl or ethyl;
R³ is C₁₋₃ alkyl optionally substituted with C₁₋₃ alkoxy; and
each of R^{2a}, R^{2b}, R^{4a}, R^{4b}, R^{6a}, R^{6b}, R^{11a}, R^{11b}, R^{16a}, and R^{16b} is hydrogen.

56. The compound or pharmaceutically acceptable salt of 55, wherein [symbol] is a single bond.

57. The compound or pharmaceutically acceptable salt of claim 55, wherein R⁵ is hydrogen or methyl in the cis position relative to the R¹⁹ group.

58. The compound or pharmaceutically acceptable salt of claim 55, wherein R⁵ is hydrogen or methyl in the trans position relative to the R¹⁹ group.

59. The compound or pharmaceutically acceptable salt of claim 55, wherein [symbol] is a double bond, and R⁵ and one of R^{6a} or R^{6b} is absent.

60. The compound or pharmaceutically acceptable salt of claim 55, wherein R¹⁸ is methyl.

61. The compound or pharmaceutically acceptable salt of claim 55, wherein R¹⁸ is ethyl.

62. The compound or pharmaceutically acceptable salt of claim 55, wherein R¹⁹ is hydrogen, methyl, or ethyl.

63. The compound or pharmaceutically acceptable salt of claim 55, wherein R¹⁹ is hydrogen.

64. The compound or pharmaceutically acceptable salt of claim 55, wherein R¹⁹ is ethyl.

65. The compound or pharmaceutically acceptable salt of claim 55, wherein R¹⁹ is cyclopropyl.

66. The compound or pharmaceutically acceptable salt of claim 55, wherein R³ is unsubstituted C₁₋₃ alkyl.

67. The compound or pharmaceutically acceptable salt of claim 55, wherein R³ is methyl optionally substituted with C₁₋₃ alkoxy.

68. The compound or pharmaceutically acceptable salt of claim 55, wherein R³ is selected from methyl, ethyl, propyl, methoxymethyl, or ethoxymethyl.

69. The compound or pharmaceutically acceptable salt of claim 55, wherein n is 1, and X is hydrogen.

70. The compound or pharmaceutically acceptable salt of claim 55, wherein n is 2, and X is hydrogen.

71. The compound or pharmaceutically acceptable salt of claim 55, wherein n is 1, and X is a 5-6 membered monocyclic heteroaryl having 2-4 nitrogen atoms, wherein the heteroaryl is optionally substituted with one group selected from halo, C₁₋₃ alkyl, —CN, or C₁₋₃ alkoxy.

72. The compound or pharmaceutically acceptable salt of claim 71, wherein X is

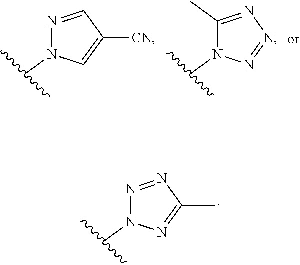

73. The compound or pharmaceutically acceptable salt of claim 72, wherein X is

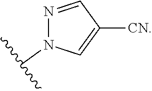 [SMILES: *n1cc(C#N)cn1]

74. A compound selected from the group consisting of:

| Example | STRUCTURE |
|---|---|
| 19 | 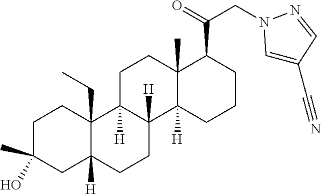 |
| 20 | 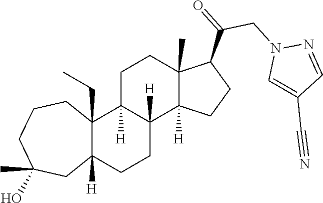 |
| 21 | 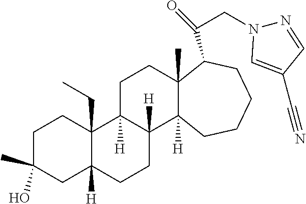 |
| 22 | 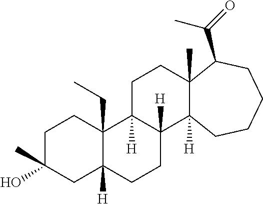 |
| 23 | 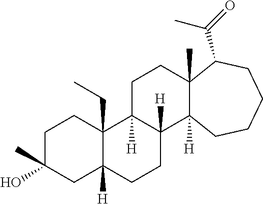 |
| 24 | 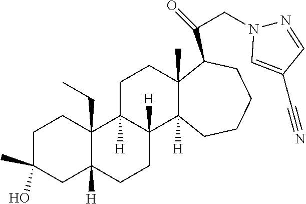 |
| 25 | 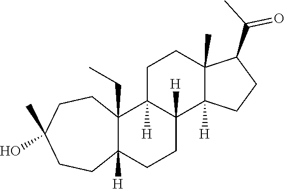 |
| 26 | 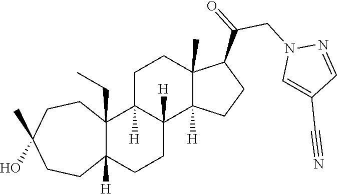 |
| 27 | 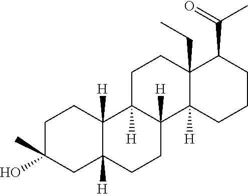 |
| 28 | 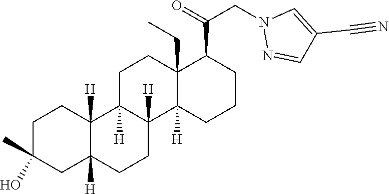 |
| 29 | 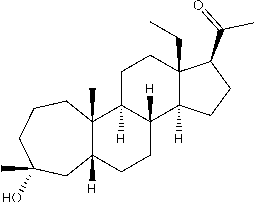 |
| 30 | 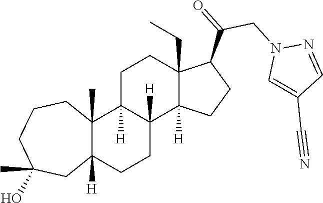 |
| 31 | 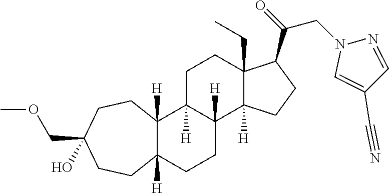 |
| 32 | 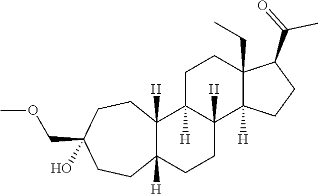 |
| 33 | 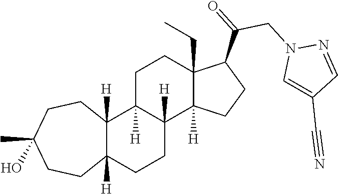 |
| 34 | 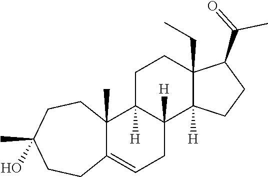 |
| 35 | 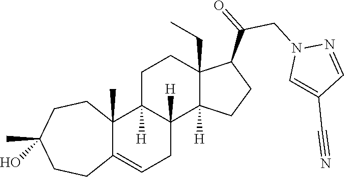 |
| 85 | 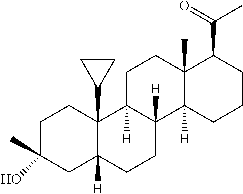 |
| 86 | 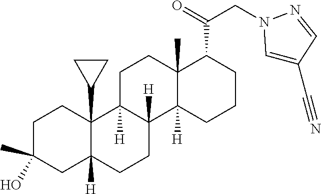 |
| 87 | 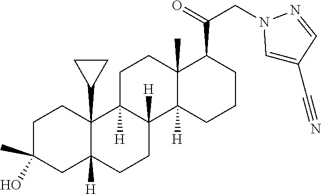 |
| 88 | 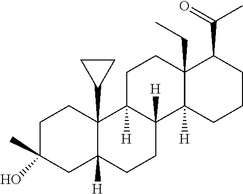 |
| 89 | 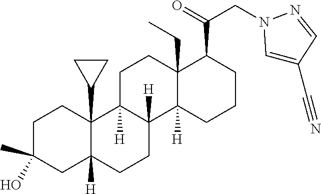 |
| 90 | 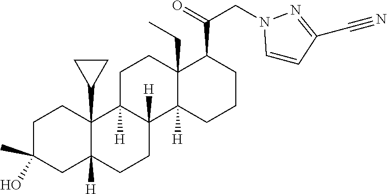 |
| 91 | 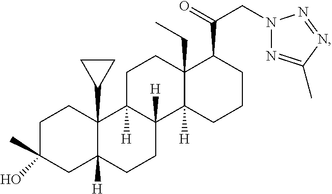 |
| | and |
| 92 | 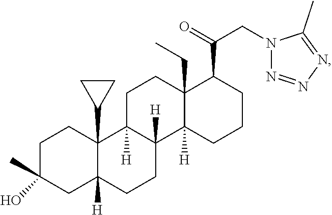 |

or a pharmaceutically acceptable salt thereof.

75. The compound or pharmaceutically acceptable salt of claim 74, wherein the compound or pharmaceutically acceptable salt is a compound selected from the group consisting of:

| Example | STRUCTURE |
|---|---|
| 19 | 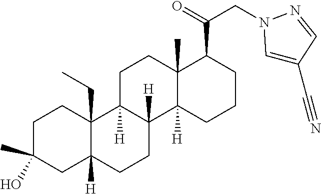 |
| 20 | 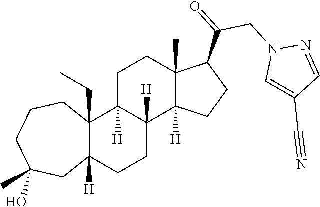 |
| 21 | 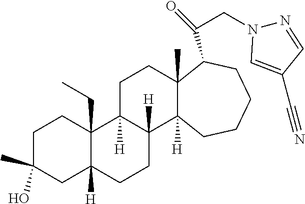 |
| 22 | 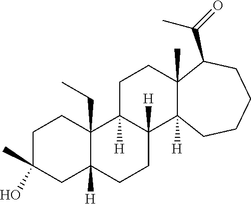 |
| 23 | 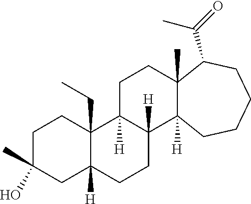 |
| 24 | 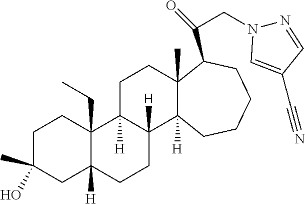 |
| 25 | 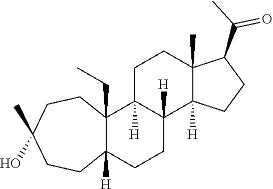 |
| 26 | 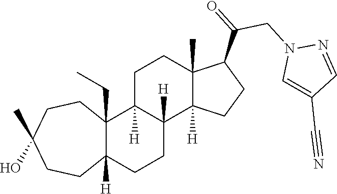 |
| 27 | 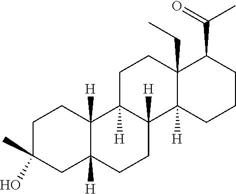 |
| 28 | 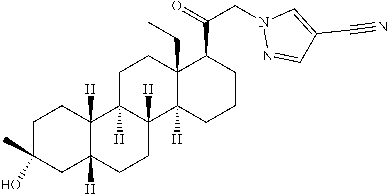 |
| 29 | 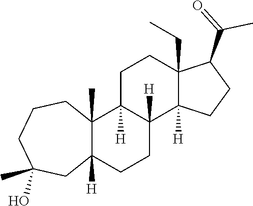 |
| 30 | 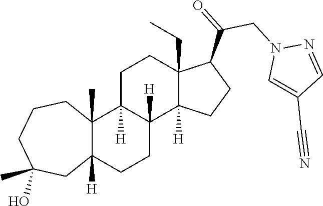 |
| 31 | 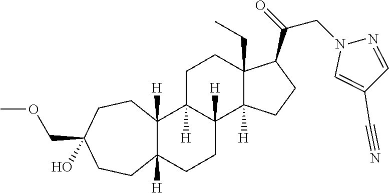 |
| 32 | 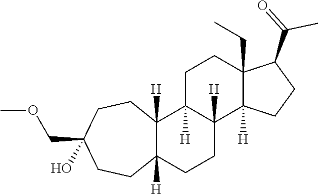 |
| 33 | 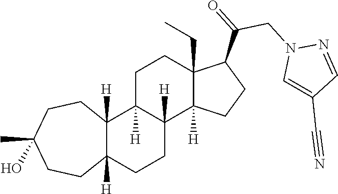 |
| 34 | 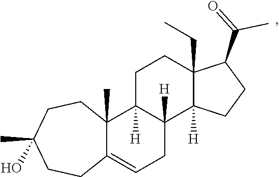 |
| | and |
| 35 | 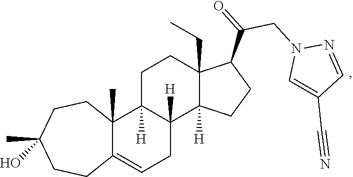 |

or a pharmaceutically acceptable salt thereof.

76. The compound or pharmaceutically acceptable salt of claim 74, wherein the compound or pharmaceutically acceptable salt is a compound selected from the group consisting of:

| Example | STRUCTURE |
|---|---|
| 85 | 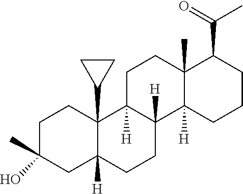 |
| 86 | 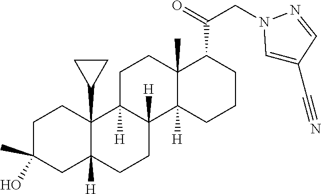 |
| 87 | 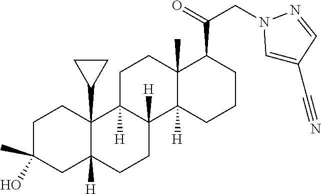 |
| 88 | 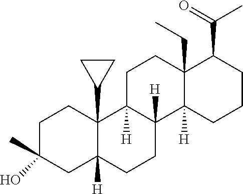 |
| 89 | 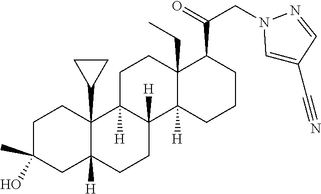 |
| 90 | 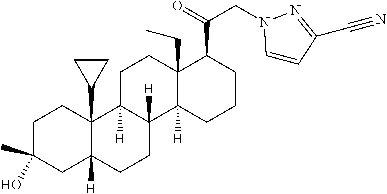 |
| 91 | 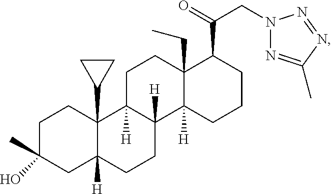 |
| | and |
| 92 | 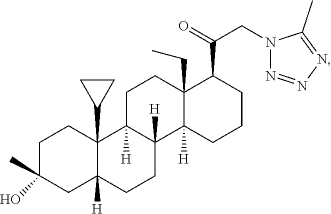 |

or a pharmaceutically acceptable salt thereof.

77. The compound or pharmaceutically acceptable salt of claim 74, wherein the compound or pharmaceutically acceptable salt is a compound selected from the group consisting of:

| Example | STRUCTURE |
|---|---|
| 19 | 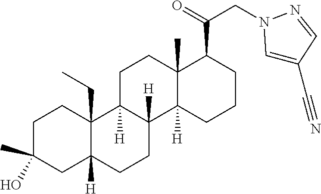 |
| 20 | 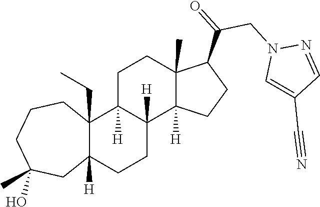 |
| 21 | 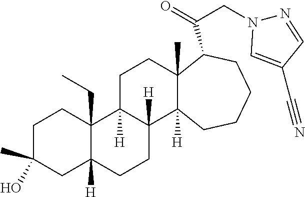 |
| 22 | 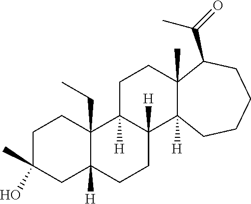 |
| 23 | 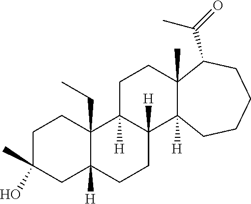 |
| 24 | 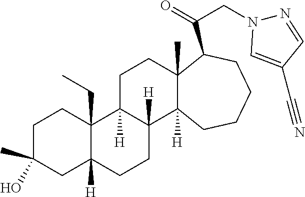 |
| 25 | 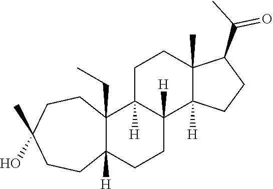 |
| 26 | 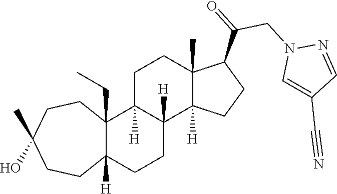 |
| 27 | 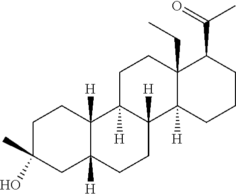 |
| 28 | 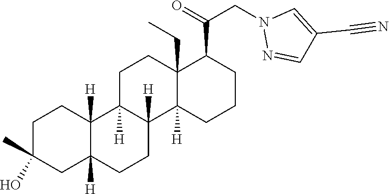 |
| 29 | 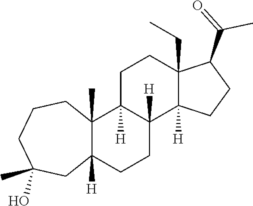 |
| 30 | 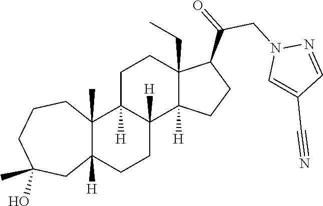 |
| 31 | 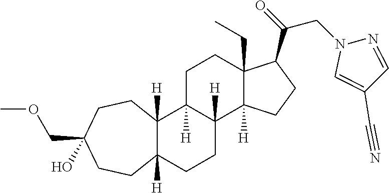 |
| 32 | 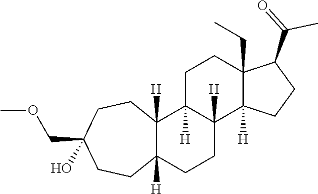 |
| 33 | 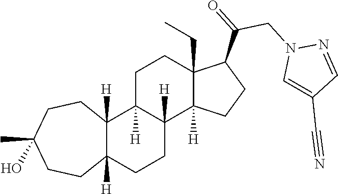 |
| 34 | 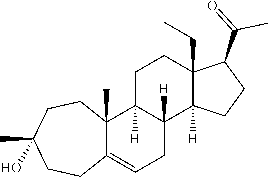 |
| 35 | 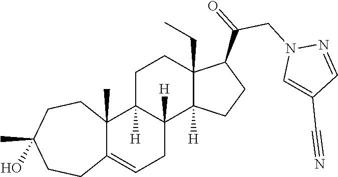 |
| 85 | 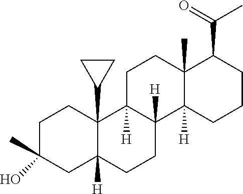 |
| 86 | 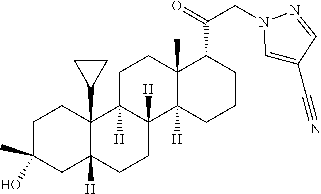 |
| 87 | 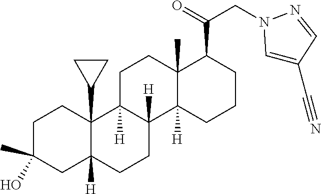 |
| 88 | 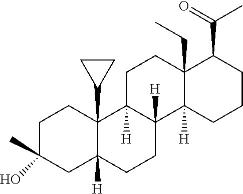 |
| 89 | 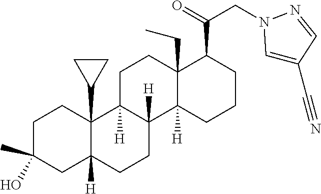 |
| 90 | 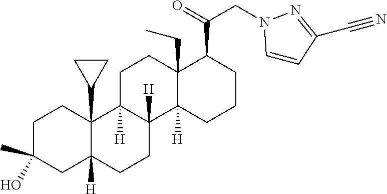 |
| 91 | 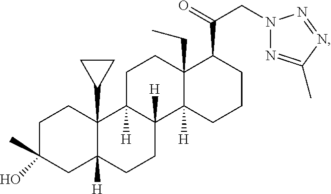 |
| | and |
| 92 | 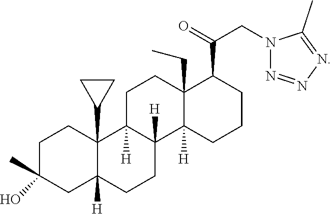 |

78. A pharmaceutical composition comprising the compound or pharmaceutically acceptable salt of claim 55 and a pharmaceutically acceptable excipient.

79. A pharmaceutical composition comprising a compound or pharmaceutically acceptable salt of claim 74 and a pharmaceutically acceptable excipient.

80. A method of treating a CNS-related disorder in a subject in need thereof, comprising administering to the subject an effective amount of the compound or pharmaceutically acceptable salt of claim 55.
